SEQUENCE LISTING (8 sequences)

SEQ ID NO: 1 - RNA, 7747 nt, synthetic construct
  source 1..7747: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaa

SEQ ID NO: 2 - DNA, 10000 bp, synthetic construct
  source 1..10000: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 3 - DNA, 24 bp, synthetic construct
  source 1..24: mol_type = other DNA
ctctctacggctaacctgaatgga

SEQ ID NO: 4 - DNA, 20 bp, synthetic construct
  source 1..20: mol_type = other DNA
aatgatacggcgaccaccga

SEQ ID NO: 5 - DNA, 3822 bp, synthetic construct
  source 1..3822: mol_type = other DNA
atgtttgtttttcttgttcttctgccattggtgtcttcccaatgtgtgaacttgactacccgcacacagctcccgccggcgtacactaactccttcacccggggggtttactacccggacaaagtgttccgcagcagtgtcctgcattcaacacaagatttgttccttccattctttagcaatgtcacttggttccatgctatacacgtgagtggcactaacggcactaagcgcttcgataacccggttctgccgtttaatgatggcgtttacttcgcaagtactgagaaatcaaacattatacgggggtggatattcggtactacattggatagtaaaactcaaagccttttgattgtcaacaatgcaactaatgtagtcataaaagtttgtgaattccaattctgtaacgatccctttcttggtgtgtattaccacaagaataacaaatcatggatggagtctgaatttcgagtatacagtagcgccaataattgcacgtttgagtatgtaagccagccgtttctgatggacttggaaggaaagcaaggcaactttaagaacctcagggagttcgtattcaaaaatatagacggttacttcaagatttactcaaaacatacacctatcaacttggtccgagatctcccacaaggtttttcagcgctcgagccattggtggacctgcccatcggaatcaacattacccgattccagacactgcttgcactgcaccggagttatctcacacccggcgacagctcgtctggttggacggcgggagctgccgcttactatgtcggttacttgcaacctcggacatttcttctgaagtataacgaaaacggtacgatcaccgatgccgtagactgcgctctcgatcccctttcagagacgaagtgcacgctcaaatcatttactgtggaaaaaggaatttatcaaacgagcaatttcagggtgcaacccacggagtccatcgtacgattcccaaacataacaaatctttgcccgtttggcgaggtttttaatgctacgcgctttgcatcagtgtacgcttggaataggaaacgaatctcaaattgtgttgcggattattccgtgttgtataattccgcaagcttctccacctttaaatgttatggcgtgtcccccacaaaacttaacgatctttgctttacaaatgtctatgcggattcattcgttatccgaggggatgaagtgcgacaaatcgctcctgggcagacagggaagatagctgattacaattacaaacttccagacgattttacaggatgtgtaatcgcgtggaactcaaacaatttggacagtaaggttggtggcaactacaattatctgtatagacttttcagaaaaagtaacctcaaacctttcgaacgcgacattagcactgagatataccaggccggatcaactccttgtaacggtgttgaaggtttcaactgttactttccgctccaatcctacggctttcaaccaacgaacggtgtcgggtaccaaccatacagagttgtcgtcttgagttttgagcttttgcacgcacctgcaaccgtgtgtggtccgaagaaaagtactaatctggttaaaaataaatgcgtcaatttcaactttaatgggttgacgggcacaggcgtgctgacagagtccaataaaaagtttttgccctttcagcagtttgggagggacatcgccgatacgaccgatgccgtccgagatcctcaaacccttgaaatattggacattaccccctgtagttttggcggtgtaagtgtgattactcccggaacgaatacatctaaccaggtagcggtgctttatcaagatgtcaactgtacagaggtccccgtggcgattcacgccgatcagttgacacccacatggagggtttacagcacaggatccaacgtcttccagacgagagcaggatgcttgatcggtgccgagcatgtgaataacagctacgaatgtgacattccgattggtgctggaatctgcgcaagttaccaaactcaaacgaattcccctggctccgcctcctctgtggcatcccagtctataatcgcgtatacgatgtcactgggcgccgagaattctgtcgcatatagcaacaactccatcgcaatacccacaaattttacaatttctgtgaccacggaaattctgcccgtcagcatgacaaaaactagcgtggactgcacgatgtatatctgcggcgactccacagagtgttccaacctcctcttgcaatatggcagcttttgcacacaattgaaccgagcgctcaccgggatagcggtcgagcaagataagaatacccaagaggtgtttgcccaggtgaagcaaatctataagacccctccaataaaggattttggcggcttcaatttctcacaaattttgcccgatccatctaagccgtccaagcgatcatttattgaggatctgctgttcaacaaagtcactctcgccgacgcggggtttattaaacaatatggcgattgcctgggcgacatcgccgccagggaccttatatgcgcccagaagttcaacggtctcaccgtgctgccaccattgttgactgacgaaatgatcgcacagtatacgtcagcgctgctggctggtaccattacttctggctggacattcggagccggtgcagcccttcagattccatttgcaatgcagatggcctatagatttaatggaataggagtgacgcagaacgtattgtacgagaatcaaaagttgatcgcaaaccagttcaattctgccattggaaagatacaagattccctttcttctaccgcctcagccctgggtaagttgcaagatgtcgtcaaccaaaatgcacaggctttgaataccttggtaaaacaattgtcatccaatttcggcgccataagctcagtacttaacgatatcttgtcacgattggatccccccgaagccgaggtccaaatcgataggctcatcacgggacggctccagagccttcaaacctatgtgacgcaacaactgatacgggccgcagaaatccgagcgtccgccaacttggcagctaccaagatgtccgaatgtgtactgggtcaatcaaagagagttgatttctgtggaaaaggataccatctcatgtcattcccccaaagcgccccgcacggcgtcgtgttcctccatgtgacttacgttccagcacaagaaaaaaacttcacaactgcgcccgctatctgccatgacgggaaggcgcacttcccacgagagggtgtatttgtgtcaaatggaacgcactggttcgtaacgcaaaggaacttctatgaaccgcagataatcacgactgacaacacttttgtatctggcaattgtgatgtggtcatcgggatagttaacaacactgtctatgacccgctgcaacccgagcttgattcatttaaagaagaactcgataaatatttcaagaatcacaccagtcccgatgtggaccttggagacatttccgggataaatgcctctgtggtaaacatccaaaaggaaatcgatcggctgaatgaagtggccaaaaacttgaacgaaagtctgatagaccttcaggagctcgggaagtatgaacaatacataaagtggccatggtacatctggcttgggttcatcgctggcctcatagccatcgttatggttaccatcatgctctgctgtatgacttcctgttgttcctgtctcaaagggtgttgcagctgtggtagctgctgcaaatttgacgaggatgactcagaacccgtcttgaagggagtcaagttgcactacacttag

SEQ ID NO: 6 - DNA, 6412 bp, synthetic construct
  source 1..6412: mol_type = other DNA
acgcgtaatacgactcactataggggaaataagagagaaaagaagagtaagaagaaatataagaccccggcgccgccaccatgtttgtttttcttgttcttctgccattggtgtcttcccaatgtgtgaacttgactacccgcacacagctcccgccggcgtacactaactccttcacccggggggtttactacccggacaaagtgttccgcagcagtgtcctgcattcaacacaagatttgttccttccattctttagcaatgtcacttggttccatgctatacacgtgagtggcactaacggcactaagcgcttcgataacccggttctgccgtttaatgatggcgtttacttcgcaagtactgagaaatcaaacattatacgggggtggatattcggtactacattggatagtaaaactcaaagccttttgattgtcaacaatgcaactaatgtagtcataaaagtttgtgaattccaattctgtaacgatccctttcttggtgtgtattaccacaagaataacaaatcatggatggagtctgaatttcgagtatacagtagcgccaataattgcacgtttgagtatgtaagccagccgtttctgatggacttggaaggaaagcaaggcaactttaagaacctcagggagttcgtattcaaaaatatagacggttacttcaagatttactcaaaacatacacctatcaacttggtccgagatctcccacaaggtttttcagcgctcgagccattggtggacctgcccatcggaatcaacattacccgattccagacactgcttgcactgcaccggagttatctcacacccggcgacagctcgtctggttggacggcgggagctgccgcttactatgtcggttacttgcaacctcggacatttcttctgaagtataacgaaaacggtacgatcaccgatgccgtagactgcgctctcgatcccctttcagagacgaagtgcacgctcaaatcatttactgtggaaaaaggaatttatcaaacgagcaatttcagggtgcaacccacggagtccatcgtacgattcccaaacataacaaatctttgcccgtttggcgaggtttttaatgctacgcgctttgcatcagtgtacgcttggaataggaaacgaatctcaaattgtgttgcggattattccgtgttgtataattccgcaagcttctccacctttaaatgttatggcgtgtcccccacaaaacttaacgatctttgctttacaaatgtctatgcggattcattcgttatccgaggggatgaagtgcgacaaatcgctcctgggcagacagggaagatagctgattacaattacaaacttccagacgattttacaggatgtgtaatcgcgtggaactcaaacaatttggacagtaaggttggtggcaactacaattatctgtatagacttttcagaaaaagtaacctcaaacctttcgaacgcgacattagcactgagatataccaggccggatcaactccttgtaacggtgttgaaggtttcaactgttactttccgctccaatcctacggctttcaaccaacgaacggtgtcgggtaccaaccatacagagttgtcgtcttgagttttgagcttttgcacgcacctgcaaccgtgtgtggtccgaagaaaagtactaatctggttaaaaataaatgcgtcaatttcaactttaatgggttgacgggcacaggcgtgctgacagagtccaataaaaagtttttgccctttcagcagtttgggagggacatcgccgatacgaccgatgccgtccgagatcctcaaacccttgaaatattggacattaccccctgtagttttggcggtgtaagtgtgattactcccggaacgaatacatctaaccaggtagcggtgctttatcaagatgtcaactgtacagaggtccccgtggcgattcacgccgatcagttgacacccacatggagggtttacagcacaggatccaacgtcttccagacgagagcaggatgcttgatcggtgccgagcatgtgaataacagctacgaatgtgacattccgattggtgctggaatctgcgcaagttaccaaactcaaacgaattcccctggctccgcctcctctgtggcatcccagtctataatcgcgtatacgatgtcactgggcgccgagaattctgtcgcatatagcaacaactccatcgcaatacccacaaattttacaatttctgtgaccacggaaattctgcccgtcagcatgacaaaaactagcgtggactgcacgatgtatatctgcggcgactccacagagtgttccaacctcctcttgcaatatggcagcttttgcacacaattgaaccgagcgctcaccgggatagcggtcgagcaagataagaatacccaagaggtgtttgcccaggtgaagcaaatctataagacccctccaataaaggattttggcggcttcaatttctcacaaattttgcccgatccatctaagccgtccaagcgatcatttattgaggatctgctgttcaacaaagtcactctcgccgacgcggggtttattaaacaatatggcgattgcctgggcgacatcgccgccagggaccttatatgcgcccagaagttcaacggtctcaccgtgctgccaccattgttgactgacgaaatgatcgcacagtatacgtcagcgctgctggctggtaccattacttctggctggacattcggagccggtgcagcccttcagattccatttgcaatgcagatggcctatagatttaatggaataggagtgacgcagaacgtattgtacgagaatcaaaagttgatcgcaaaccagttcaattctgccattggaaagatacaagattccctttcttctaccgcctcagccctgggtaagttgcaagatgtcgtcaaccaaaatgcacaggctttgaataccttggtaaaacaattgtcatccaatttcggcgccataagctcagtacttaacgatatcttgtcacgattggatccccccgaagccgaggtccaaatcgataggctcatcacgggacggctccagagccttcaaacctatgtgacgcaacaactgatacgggccgcagaaatccgagcgtccgccaacttggcagctaccaagatgtccgaatgtgtactgggtcaatcaaagagagttgatttctgtggaaaaggataccatctcatgtcattcccccaaagcgccccgcacggcgtcgtgttcctccatgtgacttacgttccagcacaagaaaaaaacttcacaactgcgcccgctatctgccatgacgggaaggcgcacttcccacgagagggtgtatttgtgtcaaatggaacgcactggttcgtaacgcaaaggaacttctatgaaccgcagataatcacgactgacaacacttttgtatctggcaattgtgatgtggtcatcgggatagttaacaacactgtctatgacccgctgcaacccgagcttgattcatttaaagaagaactcgataaatatttcaagaatcacaccagtcccgatgtggaccttggagacatttccgggataaatgcctctgtggtaaacatccaaaaggaaatcgatcggctgaatgaagtggccaaaaacttgaacgaaagtctgatagaccttcaggagctcgggaagtatgaacaatacataaagtggccatggtacatctggcttgggttcatcgctggcctcatagccatcgttatggttaccatcatgctctgctgtatgacttcctgttgttcctgtctcaaagggtgttgcagctgtggtagctgctgcaaatttgacgaggatgactcagaacccgtcttgaagggagtcaagttgcactacacttagtaatctagacggcgcgcccacccagcggccgctgataataggctggagcctcggtggccatgcttcttgccccttgggcctccccccagcccctcctccccttcctgcacccgtacccccgtggtctttgaataaagtctgattttcattgcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcac

SEQ ID NO: 7 - RNA, 4157 nt, synthetic construct
  source 1..4157: mol_type = other RNA
aggggaaataagagagaaaagaagagtaagaagaaatataagaccccggcgccgccaccatgtttgtttttcttgttcttctgccattggtgtcttcccaatgtgtgaacttgactacccgcacacagctcccgccggcgtacactaactccttcacccggggggtttactacccggacaaagtgttccgcagcagtgtcctgcattcaacacaagatttgttccttccattctttagcaatgtcacttggttccatgctatacacgtgagtggcactaacggcactaagcgcttcgataacccggttctgccgtttaatgatggcgtttacttcgcaagtactgagaaatcaaacattatacgggggtggatattcggtactacattggatagtaaaactcaaagccttttgattgtcaacaatgcaactaatgtagtcataaaagtttgtgaattccaattctgtaacgatccctttcttggtgtgtattaccacaagaataacaaatcatggatggagtctgaatttcgagtatacagtagcgccaataattgcacgtttgagtatgtaagccagccgtttctgatggacttggaaggaaagcaaggcaactttaagaacctcagggagttcgtattcaaaaatatagacggttacttcaagatttactcaaaacatacacctatcaacttggtccgagatctcccacaaggtttttcagcgctcgagccattggtggacctgcccatcggaatcaacattacccgattccagacactgcttgcactgcaccggagttatctcacacccggcgacagctcgtctggttggacggcgggagctgccgcttactatgtcggttacttgcaacctcggacatttcttctgaagtataacgaaaacggtacgatcaccgatgccgtagactgcgctctcgatcccctttcagagacgaagtgcacgctcaaatcatttactgtggaaaaaggaatttatcaaacgagcaatttcagggtgcaacccacggagtccatcgtacgattcccaaacataacaaatctttgcccgtttggcgaggtttttaatgctacgcgctttgcatcagtgtacgcttggaataggaaacgaatctcaaattgtgttgcggattattccgtgttgtataattccgcaagcttctccacctttaaatgttatggcgtgtcccccacaaaacttaacgatctttgctttacaaatgtctatgcggattcattcgttatccgaggggatgaagtgcgacaaatcgctcctgggcagacagggaagatagctgattacaattacaaacttccagacgattttacaggatgtgtaatcgcgtggaactcaaacaatttggacagtaaggttggtggcaactacaattatctgtatagacttttcagaaaaagtaacctcaaacctttcgaacgcgacattagcactgagatataccaggccggatcaactccttgtaacggtgttgaaggtttcaactgttactttccgctccaatcctacggctttcaaccaacgaacggtgtcgggtaccaaccatacagagttgtcgtcttgagttttgagcttttgcacgcacctgcaaccgtgtgtggtccgaagaaaagtactaatctggttaaaaataaatgcgtcaatttcaactttaatgggttgacgggcacaggcgtgctgacagagtccaataaaaagtttttgccctttcagcagtttgggagggacatcgccgatacgaccgatgccgtccgagatcctcaaacccttgaaatattggacattaccccctgtagttttggcggtgtaagtgtgattactcccggaacgaatacatctaaccaggtagcggtgctttatcaagatgtcaactgtacagaggtccccgtggcgattcacgccgatcagttgacacccacatggagggtttacagcacaggatccaacgtcttccagacgagagcaggatgcttgatcggtgccgagcatgtgaataacagctacgaatgtgacattccgattggtgctggaatctgcgcaagttaccaaactcaaacgaattcccctggctccgcctcctctgtggcatcccagtctataatcgcgtatacgatgtcactgggcgccgagaattctgtcgcatatagcaacaactccatcgcaatacccacaaattttacaatttctgtgaccacggaaattctgcccgtcagcatgacaaaaactagcgtggactgcacgatgtatatctgcggcgactccacagagtgttccaacctcctcttgcaatatggcagcttttgcacacaattgaaccgagcgctcaccgggatagcggtcgagcaagataagaatacccaagaggtgtttgcccaggtgaagcaaatctataagacccctccaataaaggattttggcggcttcaatttctcacaaattttgcccgatccatctaagccgtccaagcgatcatttattgaggatctgctgttcaacaaagtcactctcgccgacgcggggtttattaaacaatatggcgattgcctgggcgacatcgccgccagggaccttatatgcgcccagaagttcaacggtctcaccgtgctgccaccattgttgactgacgaaatgatcgcacagtatacgtcagcgctgctggctggtaccattacttctggctggacattcggagccggtgcagcccttcagattccatttgcaatgcagatggcctatagatttaatggaataggagtgacgcagaacgtattgtacgagaatcaaaagttgatcgcaaaccagttcaattctgccattggaaagatacaagattccctttcttctaccgcctcagccctgggtaagttgcaagatgtcgtcaaccaaaatgcacaggctttgaataccttggtaaaacaattgtcatccaatttcggcgccataagctcagtacttaacgatatcttgtcacgattggatccccccgaagccgaggtccaaatcgataggctcatcacgggacggctccagagccttcaaacctatgtgacgcaacaactgatacgggccgcagaaatccgagcgtccgccaacttggcagctaccaagatgtccgaatgtgtactgggtcaatcaaagagagttgatttctgtggaaaaggataccatctcatgtcattcccccaaagcgccccgcacggcgtcgtgttcctccatgtgacttacgttccagcacaagaaaaaaacttcacaactgcgcccgctatctgccatgacgggaaggcgcacttcccacgagagggtgtatttgtgtcaaatggaacgcactggttcgtaacgcaaaggaacttctatgaaccgcagataatcacgactgacaacacttttgtatctggcaattgtgatgtggtcatcgggatagttaacaacactgtctatgacccgctgcaacccgagcttgattcatttaaagaagaactcgataaatatttcaagaatcacaccagtcccgatgtggaccttggagacatttccgggataaatgcctctgtggtaaacatccaaaaggaaatcgatcggctgaatgaagtggccaaaaacttgaacgaaagtctgatagaccttcaggagctcgggaagtatgaacaatacataaagtggccatggtacatctggcttgggttcatcgctggcctcatagccatcgttatggttaccatcatgctctgctgtatgacttcctgttgttcctgtctcaaagggtgttgcagctgtggtagctgctgcaaatttgacgaggatgactcagaacccgtcttgaagggagtcaagttgcactacacttagtaatctagacggcgcgcccacccagcggccgctgataataggctggagcctcggtggccatgcttcttgccccttgggcctccccccagcccctcctccccttcctgcacccgtacccccgtggtctttgaataaagtctgattttcattgcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 20 bp, synthetic construct
  source 1..20: mol_type = other DNA
tcggtggtcgccgtatcatt

(19) United States
(12) Patent Application Publication
(10) Pub. No.: US 2024/0189417 A1
(43) Pub. Date: Jun. 13, 2024

(54) SUBSTITUTION OF NUCLEOTIDE BASES IN SELF-AMPLIFYING MESSENGER RIBONUCLEIC ACIDS

(71) Applicant: GlaxoSmithKline Biologicals SA, Rixensart (BE)

(72) Inventors: Giulietta MARUGGI, Rockville, MD (US); Qiongman KONG, Rockville, MD (US); Varnika ROY, Rockville, MD (US); Dong YU, Rockville, MD (US); Meng ZHANG, Rockville, MD (US)

(73) Assignee: GlaxoSmithKline Biologicals SA, Rixensart (BE)

(21) Appl. No.: 18/425,426

(22) Filed: Jan. 29, 2024

Related U.S. Application Data
(63) Continuation of application No. PCT/IB2022/058233, filed on Sep. 1, 2022.
(60) Provisional application No. 63/240,502, filed on Sep. 3, 2021.

(51) Int. Cl. A61K 39/215; A61K 39/00; A61K 39/02; A61P 31/14; A61P 37/04; C12P 19/34

(52) U.S. Cl. CPC A61K 39/215; A61K 39/0002; A61K 39/0011; A61K 39/0013; A61K 39/02; A61P 31/14; A61P 37/04; C12P 19/34; A61K 2039/53

(57) ABSTRACT

Provided herein are RNA that: are collectively self-amplifying in an intracellular environment, comprise N1-methyl-pseudouridines and uridines, and have a mole percentage or mole proportion of the N1-methylpseudouridines to the total of the uridines and the Ni-methylpseudouridines or a mole ration of the N1-methylpseudouridines to the uridines.

### CROSS-REFERENCE TO RELATED APPLICATIONS

This application claims the benefit of U.S. Provisional Application No. 63/240,502, filed Sep. 3, 2021, the complete contents of which are hereby incorporated herein by reference for all purposes.

### SEQUENCE LISTING

The instant application contains a Sequence Listing, which has been submitted electronically in computer readable form in XML file format and is hereby incorporated by reference in its entirety. Said XML file, created on Aug. 31, 2022, is named “70031WO01_SL.xml” and is 39,961 bytes in size. The information recorded in the above-identified computer readable form is identical to the written-on-paper sequence listing.

### FIELD OF THE INVENTION

Provided herein are RNA that: are collectively self-amplifying in an intracellular environment, comprise N1-methylpseudouridines and uridines, and have a mole percentage or mole proportion of the N1-methylpseudouridines to the total of the uridines and the N1-methylpseudouridines or a mole ratio of the N1-methylpseudouridines to the uridines.

### BACKGROUND TO THE INVENTION

Self-amplifying messenger (SAM) ribonucleic acid (RNA) molecules and pluralities of messenger RNA (mRNA) that collectively have the properties of a SAM RNA molecule (called a plurality of auto-amplifying messenger (AAM) RNA herein) have the capacity to provide a cell transfected with them longer exposure to exogenous or heterologous nucleic acids, including heterologous proteins such as antigens, antibodies, etc. encoded thereby. For example, SAM RNA platforms can cause antigen expression in vivo for around 30 days, whereas conventional (non-replicating) mRNA can cause expression for at most several days. However, like all exogenous RNA, SAM RNA and the pluralities of AAM RNA have the capacity to activate the innate immune system. Innate immune activation would potentially cause unintended effects apart from those related to introduction of the heterologous (or exogenous) nucleic acids. The innate immune activation could also suppress the activity of the heterologous nucleic acids introduced by the SAM RNA or the plurality of the AAM RNA. For example, the innate immune activation could suppress expression of an antigen encoded by the heterologous nucleic acid of the SAM RNA or the plurality of AAM RNA.

Substitution of uridines with N1-methylpseudouridines has been used for conventional RNA. However, conventional RNA does not require a replicase to amplify the molecule, and conventional RNA does not have persistent expression beyond the above-noted temporal maximum.

### SUMMARY OF THE INVENTION

The inventors discovered that for a self-amplifying messenger (SAM) ribonucleic acid (RNA) or a plurality of auto-amplifying messenger (AAM) RNAs there are two independent thresholds sensitive to the mole percent substitution of uridines (U) with N1-methylpseudouridines (N1Ψ). Accordingly, the inventors researched and identified the following aspects and embodiments, including the following compositions of matter, methods, or uses:

A self-amplifying messenger (SAM) ribonucleic acid (RNA) comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A plurality of auto-amplifying messenger (AAM) RNAs comprising N1-methylpseudouridines, uridines, a first RNA, and one or more second RNA; the first RNA comprising a heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.

A plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A composition comprising a pharmaceutically acceptable delivery vehicle and a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A composition comprising a pharmaceutically acceptable delivery vehicle and a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A composition comprising a pharmaceutically acceptable delivery vehicle and a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.

A composition comprising a pharmaceutically acceptable delivery vehicle and a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A method of eliciting an immune response in a subject to an immunogen, the method comprising administering to the subject an effective amount of a SAM RNA comprising N1-methylpseudouridines, uridines, a first RNA segment, and a second RNA segment; the first RNA segment comprising a heterologous nucleic acid encoding at least the immunogen or an antibody against the immunogen; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A method of eliciting an immune response in a subject to an immunogen, the method comprising administering to the subject an effective amount of a SAM RNA comprising N1-methylpseudouridines, uridines, a first RNA segment, and a second RNA segment; the first RNA segment comprising a heterologous nucleic acid encoding at least the immunogen or an antibody against the immunogen; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A method of eliciting an immune response in a subject to an immunogen, the method comprising administering to the subject an effective amount of a plurality of AAM RNAs comprising N1-methylpseudouridines, uridines, a first RNA, and one or more second RNA; the first RNA comprising a heterologous nucleic acid encoding at least the immunogen or an antibody against the immunogen; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A method of eliciting an immune response in a subject to an immunogen, a plurality of auto-amplifying messenger (AAM) RNAs comprising N1-methylpseudouridines, uridines, a first RNA, and one or more second RNA; the first RNA comprising a heterologous nucleic acid encoding at least the immunogen or an antibody against the immunogen; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.

A method of delivering an inhibitory RNA to a subject, the method comprising administering to the subject an effective amount of a SAM RNA comprising N1-methylpseudouridines, uridines, a first RNA segment, and a second RNA segment; the first RNA segment comprising a heterologous nucleic acid comprising the inhibitory RNA; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A method of delivering an inhibitory RNA, the method comprising administering to the subject an effective amount of a SAM RNA comprising N1-methylpseudouridines, uridines, a first RNA segment, and a second RNA segment; the first RNA segment comprising a heterologous nucleic acid comprising the inhibitory RNA; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A method of delivering an inhibitory RNA, the method comprising administering to the subject an effective amount of a plurality of AAM RNAs comprising N1-methylpseudouridines, uridines, a first RNA, and one or more second RNA; the first RNA comprising a heterologous nucleic acid comprising the inhibitory RNA; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.

A method of delivering an inhibitory RNA, the method comprising administering to the subject an effective amount of a plurality of AAM RNAs comprising N1-methylpseudouridines, uridines, a first RNA, and one or more second RNA; the first RNA comprising a heterologous nucleic acid comprising the inhibitory RNA; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A method of delivering to a subject a heterologous nucleic acid, the method comprising administering to the subject an effective amount of a SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A method of delivering to a subject a heterologous nucleic acid, the method comprising administering an effective amount of a SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A method of delivering to a subject a heterologous nucleic acid, the method comprising administering an effective amount of a plurality of AAM RNAs comprising N1-methylpseudouridines, uridines, a first RNA, and one or more second RNA; the first RNA comprising the heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.

A method of delivering to a subject a heterologous nucleic acid, the method comprising administering an effective amount of a plurality of AAM RNAs comprising N1-methylpseudouridines, uridines, a first RNA, and one or more second RNA; the first RNA comprising the heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A method of manufacturing a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%; the method comprising admixing an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the admixing being under conditions wherein the RNA polymerase produces the SAM RNA from the template nucleic acid.

A method of manufacturing a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A method of manufacturing a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.

A method of manufacturing a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%, the method comprising one or more admixing of an RNA polymerase, the N1-methylpseudouridines, the uridines, and one or more template nucleic acids, thereby obtaining an admixture wherein the one or more template nucleic acids comprise a sequence of the plurality of AAM RNAs; the one or more admixture having a second mole percentage of the N1-methylpseudouridines to the total of the N1-methylpseudouridines and the uridines; the second mole percentage being the same as the first mole percentage; the one or more admixing being under conditions wherein the RNA polymerase produces the plurality of AAM RNAs from the one or more template nucleic acids.

A method of manufacturing a composition comprising a pharmaceutically acceptable delivery vehicle and a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%, the method comprising admixing the pharmaceutically acceptable vehicle and the SAM RNA.

A method of manufacturing a composition comprising a pharmaceutically acceptable delivery vehicle and a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid; the method of manufacturing comprising admixing the pharmaceutically acceptable vehicle and the SAM RNA.

A method of manufacturing a composition comprising a pharmaceutically acceptable delivery vehicle and a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%; the method of manufacturing comprising admixing the pharmaceutically acceptable vehicle and the plurality of AAM RNAs.

A method of manufacturing a composition comprising a pharmaceutically acceptable delivery vehicle and a plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid; the method of manufacturing comprising admixing the pharmaceutically acceptable vehicle and the plurality of AAM RNAs.

A use of a SAM RNA for the manufacture of a medicament for delivering a heterologous nucleic acid to a subject in need thereof, the SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A use of a SAM RNA for the manufacture of a medicament for delivering a heterologous nucleic acid to a subject in need thereof, the SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%, the use comprising admixing the SAM RNA and a pharmaceutically acceptable delivery vehicle.

A use of a SAM RNA for the manufacture of a medicament for delivering a heterologous nucleic acid to a subject in need thereof, SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A use of a SAM RNA for the manufacture of a medicament for delivering a heterologous nucleic acid to a subject in need thereof, SAM RNA comprising N1-methylpseudouridines, uridines, a first RNA segment, and a second RNA segment; the first RNA segment comprising the heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid; the use comprising admixing the SAM RNA and a pharmaceutically acceptable delivery vehicle.

A use of a plurality of AAM RNAs for the manufacture of a medicament for delivering a heterologous nucleic acid to a subject in need thereof, the plurality of AAM RNAs comprising N1-methylpseudouridines, uridines, a first RNA, and one or more second RNA; the first RNA comprising the heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%. A use of a plurality of AAM RNAs for the manufacture of a medicament for delivering a heterologous nucleic acid to a subject in need thereof, the plurality of AAM RNAs comprising N1-methylpseudouridines, uridines, a first RNA, and one or more second RNA; the first RNA comprising the heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%; the use comprising admixing the plurality of AMM RNA and a pharmaceutically acceptable delivery vehicle.

A use of a plurality of AAM RNAs for the manufacture of a medicament for delivering a heterologous nucleic acid to a subject in need thereof, the plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%. A use of a plurality of AAM RNAs for the manufacture of a medicament for delivering a heterologous nucleic acid to a subject in need thereof, the plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid; the use comprising admixing the plurality of AMM RNA and a pharmaceutically acceptable delivery vehicle. A use of a SAM RNA for the manufacture of a medicament for preventing a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A use of a SAM RNA for the manufacture of a medicament for preventing a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%; the use comprising admixing the SAM RNA and a pharmaceutically acceptable delivery vehicle.

A use of a SAM RNA for the manufacture of a medicament for treating a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

A use of a SAM RNA for the manufacture of a medicament for treating a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%; the use comprising admixing the SAM RNA and a pharmaceutically acceptable delivery vehicle.

A use of a SAM RNA for the manufacture of a medicament for preventing a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid; the use comprising admixing the SAM RNA and a pharmaceutically acceptable delivery vehicle.

A use of a SAM RNA for the manufacture of a medicament for treating a disease caused by a pathogen; the pathogen comprising an immunogen; the SAM RNA comprising N1-methylpseudouridines, uridines, a first RNA segment, and a second RNA segment; the first RNA segment comprising a heterologous nucleic acid at least encoding a heterologous protein comprising the immunogen or an antibody against the immunogen; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid; the use comprising admixing the SAM RNA and a pharmaceutically acceptable delivery vehicle.

A use of a SAM RNA for the manufacture of a medicament for delivering an inhibitory RNA; the SAM comprising N1-methylpseudouridines, uridines, a first RNA segment, and a second RNA segment; the first RNA segment comprising a heterologous nucleic acid comprising the inhibitory RNA; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid; the use comprising admixing the SAM RNA and a pharmaceutically acceptable delivery vehicle.

A use of a SAM RNA for the manufacture of a medicament for delivering an inhibitory RNA; the SAM RNA comprising N1-methylpseudouridines, uridines, a first RNA segment, and a second RNA segment; the first RNA segment comprising a heterologous nucleic acid comprising the inhibitory RNA; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%; the use comprising admixing the SAM RNA and a pharmaceutically acceptable delivery vehicle.

A use of a plurality of AAM RNAs for the manufacture of a medicament for preventing a disease caused by a pathogen; the pathogen comprising an immunogen; the plurality of AAM RNAs comprising N1-methylpseudouridines, uridines, a first RNA, and one or more second RNA; the first RNA comprising a heterologous nucleic acid at least encoding a heterologous protein comprising the immunogen or an antibody against the immunogen; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%; the use comprising admixing the plurality of AAM RNAs and a pharmaceutically acceptable delivery vehicle.

A use of a plurality of AAM RNAs for the manufacture of a medicament for treating a disease caused by a pathogen; the pathogen comprising an immunogen; the plurality of AAM RNAs comprising N1-methylpseudouridines, uridines, a first RNA, and one or more second RNA; the first RNA comprising a heterologous nucleic acid at least encoding a heterologous protein comprising the immunogen or an antibody against the immunogen; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%; the use comprising admixing the plurality of the AAM RNAs and a pharmaceutically acceptable delivery vehicle.

A use of a plurality of AAM RNAs for the manufacture of a medicament for preventing a disease caused by a pathogen; the pathogen comprising an immunogen; the plurality of AAM RNAs comprising N1-methylpseudouridines, uridines, a first RNA, and one or more second RNA; the first RNA comprising a heterologous nucleic acid at least encoding a heterologous protein comprising the immunogen or an antibody against the immunogen; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid; the use comprising admixing the plurality of AAM RNAs and a pharmaceutically acceptable delivery vehicle.

A use of a plurality of AAM RNAs for the manufacture of a medicament for treating a disease caused by a pathogen; the pathogen comprising an immunogen; the plurality of AAM RNAs comprising N1-methylpseudouridines, uridines, a first RNA, and one or more second RNA; the first RNA comprising a heterologous nucleic acid at least encoding a heterologous protein comprising the immunogen or an antibody against the immunogen; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid; the use comprising admixing the plurality of AAM RNAs and a pharmaceutically acceptable delivery vehicle.

A use of a plurality of AAM RNAs for the manufacture of a medicament for delivering an inhibitory RNA; the plurality of AAM RNAs comprising N1-methylpseudouridines, uridines, a first RNA, and one or more second RNA; the first RNA comprising a heterologous nucleic acid comprising the inhibitory RNA; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%; the use comprising admixing the plurality of AAM RNAs and a pharmaceutically acceptable delivery vehicle.

A use of a plurality of AAM RNAs for the manufacture of a medicament for delivering an inhibitory RNA; the plurality of AAM RNAs comprising N1-methylpseudouridines, uridines, a first RNA, and one or more second RNA; the first RNA comprising a heterologous nucleic acid comprising the inhibitory RNA; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the use comprising admixing the plurality of AAM RNAs and a pharmaceutically acceptable delivery vehicle.

A SAM RNA for use in eliciting an immune response to an antigen in a subject; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.

A SAM RNA for use in preventing infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.

A SAM RNA for use in treating infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.

A SAM RNA for use in delivering a heterologous nucleic acid to a subject, the SAM RNA comprising N1-methylpseudorudines, uridines, a RNA segment that comprises a heterologous nucleic acid, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.

A SAM RNA for use in eliciting an immune response to an antigen in a subject; the SAM RNA comprising N1-methylpseudouridines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a mole percentage of N1-methylpseudouridines to the total of the N1-methylpseudouridines and the uridines from 15% to 75%; the admixing being under conditions wherein the RNA polymerase produces the SAM RNA from the template nucleic acid.

A SAM RNA for use in preventing infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a mole percentage of N1-methylpseudouridines to the total of the N1-methylpseudouridines and the uridines from 15% to 75%; the admixing being under conditions wherein the RNA polymerase produces the SAM RNA from the template nucleic acid.

A SAM RNA for use in treating infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A SAM RNA for use in delivering a heterologous nucleic acid to a subject, the SAM RNA comprising N1-methylpseudorudines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.

A plurality of AAM RNAs for use in eliciting an immune response to an antigen in a subject; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

A plurality of AAM RNAs for use in preventing infection by a pathogen in a subject; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

A plurality of AAM RNAs for use in treating infection by a pathogen in a subject; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

A plurality of AAM RNAs for use in delivering a heterologous nucleic acid to a subject, the plurality of AAM RNAs comprising N1-methylpseudorudines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

A plurality of AAM RNAs for use in eliciting an immune response to an antigen in a subject; the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.

A plurality of AAM RNAs for use in preventing infection by a pathogen in a subject; the pathogen producing an antigen; the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.

A plurality of AAM RNAs for use in treating infection by a pathogen in a subject; the pathogen producing an antigen; the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.

A plurality of AAM RNAs for use in delivering a heterologous nucleic acid to a subject, the plurality of AAM RNAs comprising N1-methylpseudorudines, uridines, a first RNA, and a second RNA; the first RNA comprising a heterologous nucleic acid; the second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acids.

### DESCRIPTION OF DRAWINGS

The detailed description of preferred embodiments of the invention will be better understood when read in conjunction with the appended drawings. For the purpose of illustrating the invention, the drawings show certain, but not all, preferred embodiments. It should be understood that embodiments of the invention are not limited to the precise arrangements and instrumentalities of those shown in the drawings.

FIG. 1 shows interferon-β (IFN-β) levels from C2C12 cells electroporated with 100 ng of self-amplifying messenger (SAM) ribonucleic acid (RNA) wherein 0%, 25%, 50%, 75%, or 100% of the uridines (U) are substituted with N1-methylpseudouridine (N1Ψ). Levels of IFN-β decrease in C2C12 cells administered of a SAM RNA comprising 25% substitution when compared to the levels from the same cells administered SAM RNA comprising 0% substitution. Levels further decrease with administration of SAM RNA comprising 50%, 75%, or 100% substitution compared to those with administration of SAM RNA comprising 25% substitution.

FIG. 2 depicts the percentage, as identified with fluorescence-activated cell sorting (FACS), of C2C12 cells expressing firefly luciferase encoded by SAM RNA comprising 0%, 15%, 25%, 50%, 75%, or 100% of substitution of U to N1Ψ, which was administered in a mass of 1000 ng, 333.33 ng, 111.11 ng, 37.04 ng, 12.35 ng, 4.12 ng, 1.37 ng, or 0.46 ng. The percentage of C2C12 cells expressing firefly luciferase slightly decreases with 50% substitution when compared to that with 0%, 15%, or 25% substitution and further decreases with 75% substitution when compared to that with 50% substitution. With 100% substitution, almost an undetectable percentage of cells express the firefly luciferase. When compared to the IFN-β levels shown in FIG. 1, the decreases in the expression of firefly luciferase with increasing substitution lags the decreases IFN-β levels with increasing substitution. That is, the IFN-β levels first decreases with 25% substitution, and the firefly luciferase expression first and slightly decreases with 50% substitution with 1000 ng of SAM RNA administered.

FIG. 3 depicts the intracellular signaling pathway of LEEPORTER™ luciferase reporter human embryonic kidney 293 (HEK 293) cells that starts with toll-like receptor 7 (TLR7; left) and toll-like receptor 8 (TLR8, right) activation by single-stranded ribonucleic acid (ssRNA), signals through myeloid differentiation primary response 88 (MyD88), activates p50 and p65 and then nuclear factor κ light chain enhancer of activated B cells (NF-κB), and results in renilla luciferase expression. The renilla luciferase signal is discrete from firefly luciferase signaling, which depending upon the assay is encoded by the ribonucleic acid (RNA) used to activate TLR7 or TLR8. www.abeomics.com/tlr7-nf-kb-leeporter-luciferase-reporter-hek293-cell-line; www.abeomics.com/tlr8-nf-kb-leeporter-luciferase-reporter-hek293-cell-line.

FIG. 4 shows the percent of TLR7 positive human embryonic kidney 293 (HEK293) cells (TLR7/NF-kB Leeporter™ Renilla Luciferase Reporter-HEK293 Cell Line) that express firefly luciferase upon transfection with self-amplifying messenger (SAM) riboxynucleic acid (RNA), SAM RNA deficient in the self-amplifying elements (non-replicating SAM RNA obtained with a mutation abolishing the replicase complex activity), or conventional RNA. The SAM RNA, non-replicating SAM RNA, and conventional RNA encode the firefly luciferase, have 0%, 25%, 50%, or 100% substitution of the U with N1Ψ, and are capped with cap 0 or cap 1. Increasing the amount of substitution from 0% to 25% or 50% increases the number of cells that express the firefly luciferase encoded by the exogenous SAM RNA with cap 0 or cap 1. The percent of firefly luciferase positive cells decreases with the 50% substitution compared to that with the 25% substitution. One hundred percent substitution in SAM RNA results in almost no detectable firefly luciferase positive cells regardless of whether the SAM RNA has cap 0 or cap 1, in contrast to conventional RNA that is 100% substituted.

FIG. 5 depicts total antigen expression (top, firefly luciferase expression encoded by the RNA) and TLR7 activation (bottom, renilla luciferase expression downstream of TLR7, MyD88, p50, p65, and NF-κB) with the same SAM RNA, non-replicating SAM RNA, and conventional RNA as described above for FIG. 4. Twenty five percent substitution of U to N1Ψ elevated total antigen expression above levels with 0% substitution with cap 0 and cap 1 capping. The non-replicating SAM RNA comprises an insertion in nucleic acid encoding the non-structural protein 2 thereby causing a missense translation. Increasing the substitution from 0% to 100% in non-replicating SAM RNA decreases TLR7 activation regardless of capping, thereby demonstrating that with initial administration, the substitution evades the innate immune response. With the SAM RNA (self-replicating), 25% substitution increased TLR7 activation compared to activation with 0% substitution with cap 0 capping. In combination with the results with non-replicating SAM, this demonstrates that intracellularly the self-replication incorporates non-modified forms of uridine, and these strands activate TLR7.

FIGS. 6A-6D depict dose response curves of luminescence of firefly luciferase in a polyploid human foreskin fibroblast cell line, the BJ cell line, with transfected with SAM RNA capped with cap 0 or cap 1, conventional RNA, or non-replicating SAM capped with cap 0 or cap 1. The SAM RNA, non-replicating SAM RNA, or conventional RNA encode firefly luciferase and U are 0% (FIG. 6A), 25% (FIG. 6B), 50% (FIG. 6C), or 100% (FIG. 6D) substituted with N1Ψ. Capping with cap 1 results in greater firefly luciferase luminescence in all conditions and a leftward shift in the dose response curve compared to that for cap 0 under all conditions of substitution with the SAM RNA. Antigen expression increases with 25% and 50% substitution of the SAM RNA, regardless of capping, compared that with 0% and 100% substitution of the SAM RNA or to that with conventional RNA.

FIGS. 7A-7D show dose response curves of the percent of BJ cells expressing double-stranded RNA (% of dsRNA+ cells) that replicated from the SAM RNA capped with cap 0 or cap 1, conventional RNA, or non-replicating SAM capped with cap 0 or cap 1, wherein the U are 0% (FIG. 7A), 25% (FIG. 7B), 50% (FIG. 7C), or 100% (FIG. 7D) substituted with N1Ψ (0% N1Ψ, 25% N1Ψ, 50% N1Ψ, and 100% N1Ψ respectively). Capping with cap 1 results in a leftward shift in the dose response curve compared to that with cap 0 under all conditions of substitution with the SAM RNA. The percent of BJ cells expressing double-stranded RNA plateaued to about same level (75%-78%) with 0%, 25%, or 50% substitution of U with N1Ψ with cap 1 capping. This indicates that, regardless of the percent substitution, the cap 1 capping can obtain a maximum response with a maximal dose (excepting 100% substitution). Nonetheless, 25% substitution can have an effect with cap 1 capping on the percentage of cells when compared to that with 0% substitution. When comparing the 50% mark for the percent of cells containing double-stranded RNA, there is a leftward shift with 25% substitution compared to that with 0% or 50% substitution. With cap 0 capping, 25% and 50% substitution increased the maximum percentage of cells expressing double-stranded RNA compared to that with 0% or 100% substitution. Therefore, there is an effect of substitution of U to N1Ψ on the self-amplification of SAM RNA substitution, whereby 25% or 50% substitution can increase the number of cells with self-amplification compared to that with 0% substitution, and this effect occurs within cap 0 or cap 1 capping.

FIGS. 8A-8C shows three replicates of dose response curves of total luminescence from the RNA encoding firefly luciferase with SAM RNA with cap 1 and 0%, 25%, 50%, and 100% of U to N1Ψ substitution. In each replicate, there is a leftward shift with 25% substitution compared to 0% substitution and the total amount of luciferase expressed from the SAM RNA reaches a higher maximum with 25% and 50% substitution than the maximum with 0% substitution. These results, together with the results from FIGS. 7A-7D, demonstrate that 25% and 50% substitution increase expression of amino acids encoded by the SAM RNA compared to the same measures with 0% substitution. Even if as many cells express the amino acid encoded by the SAM RNA when there is 0% substitution as when there is 25% or 50% substitution, those cells obtain increased expression of the amino acid encoded by the SAM RNA. Whether this effect is due to evasion of the innate immune system with the 25% or 50% substitution is determined in FIGS. 9A and 9B.

FIGS. 9A and 9B depict dose response curves of the percent of baby hamster kidney (BHK) cells that express double-stranded RNA (% of dsRNA+ cells) replicating from the SAM RNA capped with cap 0 (FIG. 8A) or cap 1 (FIG. 8B), conventional RNA, or non-replicating SAM capped with cap 0 or cap 1, wherein the U in the RNA are 0%, 25%, 50%, or 100% substituted with N1Ψ. BHK cells, which are deficient in innate sensing and activation, were selected to determine whether the 25% or 50% substitutions evaded the innate immune response (i.e., a TLR7-activated or TLR8-activated immune response) thereby increasing the percentage of cells having RNA that self-amplifies (FIGS. 7B and 7C) and the total amount of luminescence from the firefly luciferase (FIGS. 8A-8C) encoded by the SAM RNA when compared the same results with 0% or 100% substitution (FIGS. 7A, 7D, and 7A-7C). Increasing the substitution of U to N1Ψ from 0% to 25%, then to 50%, and then to 100% caused a rightward shift in the dose response curve regardless of whether cap 0 or cap 1 capped the SAM RNA. Therefore, evasion of the innate immune system increases the self-amplification of the SAM RNA, as measured by double-stranded RNA, and the expression of amino acids encoded by the SAM RNA observed in cells with innate immune responses with 25% or 50% substitution over the same results observed with 0% substitution. Because BHK cells are deficient in innate immune activation, these results cannot be used to infer that the improvements in self-amplification with 25% or 50% substitution are cell-specific or context-specific.

FIG. 10 depicts the percentage, as identified with FACS, of BJ cells expressing firefly luciferase encoded by SAM RNA comprising 0%, 25%, 50%, or 100% of substitution of U to N1Ψ, which was administered in a mass of 15 ng, 5.4 ng, 1.9 ng, 0.68 ng, 0.24 ng, 0.09 ng, 0.03 ng, or 0 ng. With administration of 15 ng, 5.4 ng, or 1.9 ng of replicating, 25% substituted SAM RNA, the percentage of firefly luciferase positive BJ cells increases over that with administration of replicating SAM RNA lacking substitution. With administration of 15 ng or 5.4 ng of replicating, 50% substituted SAM RNA, the percentage of firefly luciferase positive cells also increases compared to that with administration of replicating SAM RNA lacking substitution.

FIG. 11 shows interleukin 6 (IL-6) levels from BJ cells transfected with lipid nanoparticles encapsulating SAM RNA, non-replicating SAM RNA, or mRNA, wherein 0%, 25%, 50%, and 100% of U are substituted with N1Ψ and the RNA is capped with cap 1. Administration of non-replicating SAM RNA comprising 100% U to N1Ψ substitution reduces IL-6 expression compared to expression with administration of 0% substituted non-replicating SAM RNA. Therefore, increasing substitution decreases IL-6 levels. The non-replicating SAM induce lower IL-6 expression than expression with replicating SAM RNA. Self-replication of SAM RNA within a cell incorporates uridines into the newly synthesized strands, and thereby appears to increase IL-6 levels regardless of the initial substitution. Within the self-replicating SAM RNA, increasing the substitution from 0% to 25%, and then to 50%, and then to 100% reduces levels of IL-6 with each increment of substitution.

FIG. 12 depicts interferon-α (IFN-α) levels obtained from one donor of human peripheral blood monocyte cells (PBMCs) transfected with lipid nanoparticles encapsulating SAM RNA, non-replicating SAM RNA, wherein 0%, 25%, 50%, and 100% of U are substituted with N1Ψ and the RNA is capped with cap 0 or cap 1. Increasing the substitution from 0% to 25%, and then to 50%, and then to 100% in the self-replicating and non-replicating SAM RNA reduces, at each increment of substitution, the levels of IFN-α. Cap 1 capping compared to cap 0 capping reduces IFN-α levels when the hPBMC cells were exposed to 500 ng of RNA.

FIG. 13 depicts the correlation between antigen levels and double-stranded RNA levels in BJ cells transfected with 25%, 50%, and 100% U to N1Ψ RNA across varying lots of capped SAM RNA. The antigen levels and double-stranded RNA levels obtained with each of the substitutions were normalized to the same with 0% substitution, which represent levels of 100%.

FIG. 14 depicts the levels in BJ cells of double-stranded RNA obtained from transfecting the cells with SAM RNA comprising 0%, 25%, 50%, or 100% U to N1Ψ substitution and capped with cap 1. The results are normalized to the levels obtained from the cells treated with the 0% substituted SAM RNA, which represent levels of 100%. The results for the 25% and 100% substitution were from four different lots of cap 1 capping of the SAM RNA, and the results for 0% and 50% substitution were from five different lots of SAM RNA. Twenty five percent substitution increases the amount of double-stranded RNA in BJ cells compared to the same measures with 0% and 50% substitution. Fifty percent substitution slightly increases the amount of double-stranded RNA in BJ cells compared to the same measures with 0%. One hundred percent substitution results in the lowest expression of double-stranded RNA when compared to that from all other percent substitutions.

FIG. 15 depicts the levels of interleukin 6 (IL-6) from the same BJ cells transfected with SAM RNA comprising cap 1 or cap 0 capping and 0, 25%, 50%, or 100% U to N1Ψ substitution as in FIG. 14. There is no interaction between substitution and capping on IL-6 levels from BJ cells. The levels of IL-6 drop significantly with each increment of substitution in both the cap 0 and cap 1 conditions. BJ cells release less IL-6 when treated with SAM with cap 1 than when treated with SAM with cap 0.

FIG. 16 depicts the negative correlations between IL-6 levels in FIG. 15 and the double-stranded RNA levels in FIG. 14 obtained from the BJ cells transfected with SAM RNA comprising 25% or 50% U to N1Ψ substitution and cap 1 capping within different lots of cap 1. Within 25% or 50% percent substitution, the amount of IL-6 negatively correlates with the amount of double-stranded RNA. Based on these results, there are two independent thresholds sensitive to the percent substitution. The innate immune response inhibits the self-amplification of the SAM RNA. A first, lower threshold, in substitution of U to N14) triggers evasion of the innate immune response thereby improving self-amplification. There is also a direct effect of substitution on inhibiting self-amplification. Fifty percent substitution results in lower double-stranded RNA levels than levels with 25% substitution, but these levels are higher than levels with 0% substitution. One hundred percent substitution results in almost no double-stranded RNA formation. There is therefore a second, higher, threshold between 50% and 100% substitution whereby the substitution directly inhibits self-amplification. In combination, there is a balance whereby some substitution increases self-amplification by evading, or lowering, the innate immune response, but too much substitution inhibits induction of an immune response to the antigen and thereby the pathogen by inhibiting self-amplification of the SAM. Even with some inhibition of self-amplification and antigen expression from a SAM with greater than 50% substitution, as administered, will suffice to provide an effective intracellular introduction of an heterologous nucleic acid, which may encode a heterologous protein such as an antigen to induce an immune response, or a protective immune response, to the antigen, and thereby the pathogen.

FIG. 17 is the radar plot of levels of interferons-alpha and -gamma (IFN-α and IFN-γ); interleukins-6 (IL-6), -8 (IL-8), and -10 (IL-10); IFN-γ-induced protein-10 (IP-10, also known as C—X—C motif chemokine ligand-10 or CXCL-10); monocyte chemoattractant protein-1 (MCP-1); and macrophage inflammatory protein 1-beta (MIP-1β) obtained from a single donor's peripheral blood monocyte cells (PBMC) which were treated with non-replicating SAM RNA encoding firefly luciferase, comprising 0% or 100% substitutions of U to N1Ψ, and being capped with cap 1; or SAM RNA encoding firefly luciferase, comprising 0%, 50%, or 100% substitutions of U to N1Ψ, and being capped with cap 1. Fifty percent substitution, compared to 0% substitution, reduced IFN-α and IFN-γ and trended toward reducing MCP-1, IL-10, IP-10, and MIP-1β levels but not IL-6 or IL-8 levels. One hundred percent substitution, compared to 0% substitution, reduced IFN-α, IFN-γ, IL-8, IP-10, MCP-1, and MIP-1β levels, but not IL-6 levels. The reductions in IP-10 confirm the reductions in IFN-γ, since the latter induces the former. Treatment with lipopolysaccharides (a toll-like receptor 4 agonist) and R848 (also known as (a.k.a.) resiquimod, a TLR-7 and TLR-8 agonist) and the levels of IL-1α, IL-1β, IL-12, IL-18, IL-22, and tumor necrosis factor-α are not shown.

FIG. 18 depicts the histograms of levels of IFN-α, IFN-β, MCP-1, MIP-1β, and IP-10 from FIG. 17.

FIG. 19 depicts the percentage of positive human skeletal muscle cells (HSkM) upon transfection with mRNAs or SAM RNA encoding firefly luciferase and comprising 0%, 50%, or 100% substitutions of U to N1 Y and being capped with cap 1. Zero percent- and 50% substituted SAM transfect cells with similar efficiency while the 100% modified SAM is defective in self-amplification.

FIG. 20 depicts the histograms of the levels of IFN-β from the cells of FIG. 19, showing reduction of IFN-β when a 50% substituted SAM is used. In comparison to the results of FIG. 19, the decrease in IFN-β with increasing substitution precedes the decrease in gene expression encoded within the SAM RNA with increasing substitution in hSkM, as with C2C12 cells.

FIG. 21 shows the gating strategy for flow cytometry for cytokine analysis of T cells from a representative sample obtained from in vivo. Cytokines were gated on time/live/lymphocytes/singlets/CD3 CD4/CD44, or CD8/CD44. Phenotypic subsets were determined by the Boolean combination gate tool.

FIG. 22 depicts the flow cytometry gating scheme from a representative sample obtained from in vivo to identify follicular helper T cells (Tfh) cells. To measure total Tfh, cells were gated on time/live/lymphocytes/singlets/CD3/CD4/CD44/CXCR5+PD1+.

FIG. 23 shows the flow cytometry gating scheme from a representative sample from in vivo to identify SARS-CoV-2 spike protein-specific B cells and B cell phenotypes. Cells were gated on time/live/lymphocytes/singlets/CD19/IgM−IgD−. Gating for B cell phenotyping markers (CD273 (PD-L2), CD80, and CD73) were established after gating on IgM−IgD−. These gates were then applied to the spike-specific populations to get the spike-specific B cell phenotype. Germinal center (CD95+GL7+CD38−) and non-germinal center (CD95low/− GL7−CD38+) cells were gated after gating on class-switched (IgM−IgD−), and then further differentiated by cells that bind to the fluorescently-labeled SARS-CoV-2 spike antigen (spike+).

FIG. 24 shows that all treatment with each of all of the RNA designs (SAM RNA and non-replicating mRNA) elicited SARS-CoV-2 spike protein-specific antibodies. Spike protein-specific antibodies were measured in the serum at day 21 and day 35; n=13 for saline, empty LNP, and the 3 μg dose groups; n=10 for the 0.15 μg dose groups. Data are transformed into log 10 GMT values and error bars are displayed with a 95% confidence interval. The lower limit of quantitation is defined as 0.004.

FIG. 25A shows the geometric mean ratio of day 35 total SARS-CoV-2 spike protein-specific antibodies in group 4 to day 35 total SARS-CoV-2 spike protein-specific antibodies in group 3 on the left and the geometric mean ratio of total SARS-CoV-2 spike protein-specific antibodies in group 10 to the total SARS-CoV-2 spike protein-specific antibodies in group 9 on the right. The animals in group 4 received 3 μg per unit dose of unsubstituted SAM RNA with cap-1 5′ capping. The animals in group 3 received 3 μg per unit dose of unsubstituted SAM RNA with cap-0 5′ capping. The animals in group 10 received 0.15 μg per unit dose of unsubstituted SAM RNA with cap-1 5′ capping. The animals in group 9 received 0.15 μg per unit dose of unsubstituted SAM RNA with cap-0 5′ capping. The vertical dashed lines indicate a 3-fold change, whereas the vertical line indicates a ratio of 1:1.

FIG. 25B shows the geometric mean ratio of day 35 total SARS-CoV-2 spike protein-specific antibodies in group 5 to day 35 total SARS-CoV-2 spike protein-specific antibodies in group 4 on the left and the geometric mean ratio of day 35 total SARS-CoV-2 spike protein-specific antibodies in group 11 to day 35 total SARS-CoV-2 spike protein-specific antibodies in group 10 on the right. The animals in group 5 received 3 μg per unit dose of unsubstituted SAM RNA with cap-1 5′ capping, and which were oligo(dT)-cellulose column purified before LNP encapsulation. The animals in group 4 received 3 μg per unit dose of unsubstituted SAM RNA with cap-1 5′ capping and which were not oligo(dT)-cellulose column purified before LNP encapsulation. The animals in group 11 received 0.15 μg per unit dose of unsubstituted SAM RNA with cap-1 5′ capping, and which were oligo(dT)-cellulose column purified before LNP encapsulation. The animals in group 10 received 0.15 μg per unit dose of unsubstituted SAM RNA with cap-1 5′ capping, and which were not oligo(dT)-cellulose column purified before LNP encapsulation. The vertical dashed lines indicate a 3-fold change, whereas the vertical solid line indicates a ratio of 1:1.

FIG. 25C shows the geometric mean ratio of day 35 total SARS-CoV-2 spike protein-specific antibodies in groups 6 or 7 to day 35 total SARS-CoV-2 spike protein-specific antibodies in group 5 on the left and the geometric mean ratio of day 35 total SARS-CoV-2 spike protein-specific antibodies in groups 12 or 13 to day 35 total SARS-CoV-2 spike protein-specific antibodies in group 11 on the right. The animals in group 5 received 3 μg per unit dose of unsubstituted SAM RNA with cap-1 5′ capping, and which were oligo(dT)-cellulose column purified before LNP encapsulation. The animals in group 6 received 3 μg per unit dose of SAM RNA, which had 50 mole % N1-methylpseudouridine substitution and cap-1 5′ capping and which were oligo(dT)-cellulose column purified before LNP encapsulation. The animals in group 7 received 3 μg per unit dose of SAM RNA, which had 25 mole % N1-methylpseudouridine substitution and cap-1 5′ capping, and which were oligo(dT)-cellulose column purified before LNP encapsulation. The animals in group 11 received 0.15 μg per unit dose of unsubstituted SAM RNA, which had 50 mole % N1-methylpseudouridine substitution and cap-1 5′ capping and which were oligo(dT)-cellulose column purified before LNP encapsulation. The animals in group 12 received 0.15 μg per unit dose of SAM RNA, which had 50 mole % N1-methylpseudouridine substitution and cap-1 5′ capping and which were oligo(dT)-cellulose column purified before LNP encapsulation. The animals in group 13 received 0.15 μg per unit dose of SAM RNA, which had 25 mole % N1-methylpseudouridine substitution and cap-1 5′ capping and which were oligo(dT)-cellulose column purified before LNP encapsulation. The vertical dashed lines indicate a 3-fold change, whereas the vertical solid line indicates a ratio of 1:1. Substitution with 25 mole % or 50 mole % N1-methylpseudouridine did not reduce total SARS-CoV-2 spike protein-specific antibodies.

FIG. 25D shows the geometric mean ratio of day 35 total SARS-CoV-2 spike protein-specific antibodies in groups 3, 4, 5, 6, or 7 to day 35 total SARS-CoV-2 spike protein-specific antibodies in group 8 on the left and the geometric mean ratio of day 35 total SARS-CoV-2 spike protein-specific antibodies in groups 9, 10, 11, 12, or 13 to day 35 total SARS-CoV-2 spike protein-specific antibodies in group 14 on the right. The animals in group 8 received 3 μg per unit dose of mRNA (non-replicating), which had 100 mole % substitution with N1-methylpseudouridine and cap-1 5′ capping, and which were oligo(dT)-cellulose column purified before LNP encapsulation. The animals in group 14 received 0.15 μg per unit dose of mRNA (non-replicating), which had 100 mole % substitution with N1-methylpseudouridine and cap-1 5′ capping, and which were oligo(dT)-cellulose column purified before LNP encapsulation. The vertical dashed lines indicate a 3-fold change, whereas the vertical solid line indicates a ratio of 1:1. At lower unit doses of, for example, 0.15 μg, SAM RNA can outperform mRNA (non-replicating).

FIG. 26 shows SARS-CoV-2 neutralizing antibody titers (NT50) from animals treated on days 0 and 21 with the indicated LNP encapsulated RNAs (n=13 for saline, empty LNP, and the 3 μg unit dose groups and n=10 for the 0.15 μg unit dose groups). Data are transformed into log 10 NT50 and error bars are displayed with a 95% confidence interval. The lower limit of quantitation=25 (horizontal dashed line). Cap-1 5′ capping increased neutralizing antibody titers compared to that with cap-0 5′ capping. Oligo(dT)-cellulose purification did not increase neutralizing antibody titers. At a lower unit dose of, for example, 0.15 μg, administration of SAM RNA, with or without N1-methylpseudouridine substitution, can significantly increase neutralizing antibody titers when compared to those from administration of mRNA (non-replicating) which comprises N1-methylpseudouridine but not uridines.

FIG. 27A shows the geometric mean ratio of day 35 neutralizing antibody titers (NT50) in group 4 to day 35 total SARS-CoV-2 spike protein-specific antibodies in group 3 on the left and the geometric mean ratio of neutralizing antibody titers (NT50) in group 10 to the neutralizing antibody titers (NT50) in group 9 on the right. The vertical dashed lines indicate a 3-fold change, whereas the vertical line indicates a ratio of 1:1.

FIG. 27B shows the geometric mean ratio of day 35 neutralizing antibody titers (NT50) in group 5 to day 35 neutralizing antibody titers (NT50) in group 4 on the left and the geometric mean ratio of day 35 neutralizing antibody titers (NT50) in group 11 to day 35 neutralizing antibody titers (NT50) dies in group 10 on the right. The vertical dashed lines indicate a 3-fold change, whereas the vertical solid line indicates a ratio of 1:1.

FIG. 27C shows the geometric mean ratio of day 35 neutralizing antibody titers (NT50) in groups 6 or 7 to day 35 neutralizing antibody titers (NT50) in group 5 on the left and the geometric mean ratio of day 35 neutralizing antibody titers (NT50) in groups 12 or 13 to day 35 neutralizing antibody titers (NT50) in group 11 on the right. The vertical dashed lines indicate a 3-fold change, whereas the vertical solid line indicates a ratio of 1:1. Substitution with 25 mole % or 50 mole % N1-methylpseudouridine did not reduce neutralizing antibody titers (NT50).

FIG. 27D shows the geometric mean ratio of day 35 neutralizing antibody titers (NT50) in groups 3, 4, 5, 6, or 7 to day 35 neutralizing antibody titers (NT50) in group 8 on the left and the geometric mean ratio of day 35 neutralizing antibody titers (NT50) in groups 9, 10, 11, 12, or 13 to day 35 neutralizing antibody titers (NT50) in group 14 on the right. The vertical dashed lines indicate a 3-fold change, whereas the vertical solid line indicates a ratio of 1:1. At, for example, higher unit doses of 3 μg and at, for example, lower unit doses of 0.15 μg, SAM RNA can outperform mRNA (non-replicating), except for Group 3.

FIGS. 28A-28J depict serum levels of IFN-β (FIG. 28A), IFN-γ (FIG. 28B), IL-1p (FIG. 28C), IL-6 (FIG. 28D), IP-10 (FIG. 28E), keratinocyte chemoattractant/human growth-regulated oncogene (KC/GRO, FIG. 28F), MCP-1 (FIG. 28G), MIP-1β (FIG. 28H), macrophage inflammatory protein-2 (MIP-2, FIG. 28I), and TNF-α (FIG. 28J) measured 6 h (6, n=8) and 24 h (24, n=6) after the first (1) and second (2) immunizations. Data are transformed into log 10 μg/mL values and error bars are displayed as geometric mean with 95% confidence interval. The lower limit of quantitation is designated as a dotted line with the following values: 3.02 μg/mL for IFNβ, 1.56 μg/mL for IFNγ, 7.72 μg/mL for IL-1β, and 10.4 μg/mL for IL-6.

FIGS. 29A and 29B depict the results of FIGS. 28A-28J recalculated as geometric mean ratios of the results of: group 5 over those of group 4 (G5/G4), group 6 over those of group 5 (G6/G5), group 7 over those of group 5 (G7/G5), group 4 over those of group 8 (G4/G8), group 5 over those of group 8 (G5/G8), group 6 over those of group 8 (G6/G8), and group 7 over those of group 8 (G7/G8) with serum cytokine levels 6 hrs and 24 hrs after the first vaccination (V1-6 h and V1-24 h, respectively) in FIG. 29A and serum cytokine levels 6 hrs and 24 hrs after the second vaccination (V2-6 h and V2-24 h, respectively) in FIG. 29B. The leftward dashed lines indicate a three-fold reduction. The rightward dashed lines indicate a three-fold increase. The solid lines indicate no change across groups (i.e., a ratio of 1:1).

FIG. 30 shows T cell responses specific for spike protein from SARS-CoV-2. Spike-specific T cells were defined based on their Th0/Tc0, Th1/Tc1, Th2/Tc2, or Th17/Tc17 phenotype. Data are displayed as standard error of mean, frequency of grandparent (CD4 or CD8), n=5. Cytokines were gated on time/live/lymphocytes/singlets/CD3 CD4/CD44 or CD8/CD44 and phenotypic subsets were determined by the Boolean Combination Gate Tool described in Example 10. Error bars represent the median with interquartile range.

FIG. 31 shows the spike-specific CD4 Th0 cell responses of FIG. 30 as groups ratios. The vertical dashed lines indicate the 3-fold change.

FIG. 32 depicts the spike-specific CD4 Th1 cell responses of FIG. 30 as groups ratios. The vertical dashed lines indicate the 3-fold change.

FIG. 33 depicts the spike-specific CD8 Tc0 cell responses of FIG. 30 as groups ratios. The vertical dashed lines indicate the 3-fold change.

FIG. 34 depicts the spike-specific CD8 Tc1 cell responses of FIG. 30 as groups ratios. The vertical dashed lines indicate the 3-fold change.

FIG. 35 shows the proportion of follicular helper T-cells (Tfh cells) obtained from splenocytes on day 35 and measured by flow cytometry. Treatment with 3 μg unit doses of SAM RNA comprising 25 mole % N1-methylpseudouridine (N1Ψ) increased Tfh cells to levels equivalent to treatment with 3 μg unit doses of non-replicating mRNA comprising N1Ψ but not uridines. Total Tfh cells were gated on time/live/lymphocytes/singlets/CD3/CD4/CD44/CXCR5+PD1+. Data are displayed as standard error of mean, frequency of grandparent (CD4), n=5. Error bars represent the median with interquartile range.

FIG. 36 depicts the SARS-CoV-2 spike protein-specific B cell responses in in vivo germinal centers extracted on day 35. Treatment with 3 μg unit doses of SAM RNA comprising 25 mole % N1-methylpseudouridine (N1Ψ) or 3 μg unit doses of non-replicating mRNA comprising N1Ψ but not uridines elicited the highest spike protein-specific germinal center B cells. Cells were gated on time/live/lymphocytes/singlets/CD19/IgM−IgD−CD95+GL7+CD38−spike+. Data are displayed as standard error of mean, frequency of grandparent (germinal center, CD95+GL7+), n=5. Error bars represent the median with interquartile range.

FIG. 37 shows the phenotypes, as a proportion of cells, for SARS-CoV-2 spike protein-specific B cells from mice treated as described in Example 10. Treatment with 3 μg unit doses of SAM RNA comprising 25 mole % N1-methylpseudouridine (N1Ψ) or 3 μg unit doses of non-replicating mRNA comprising N1Ψ but not uridines elicited the highest levels of spike protein-specific memory B cells on day 35 of the in vivo protocol. Cells were gated on time/live/lymphocytes/singlets/CD19/IgM−IgD− CD95+GL7+CD38−spike+. Gating for B cell phenotyping markers (CD273 (PD-L2), CD80, and CD73) were established after gating on IgM−IgD−. These gates were then applied to the spike-specific populations to get the spike-specific B cell phenotype, determined by Boolean Combination Gate Tool. Data are displayed as standard error of mean, frequency of grandparent (germinal center, CD95+GL7+CD38− or non-germinal center CD95low/−GL7−CD38+), n=5. Error bars represent the median with interquartile range.

### DETAILED DESCRIPTION OF THE INVENTION

The preferred materials and methods are described herein; any methods and materials similar or equivalent to those described herein can be used in the practice of or testing of the invention.

The inventors researched and discovered that there are two independent thresholds sensitive to the mole percent substitution of uridines (U) with N1-methylpseudouridines (N1Ψ). The innate immune response inhibits the self-amplification of the SAM RNA, but the sensitivity of the innate immune response to substitution is lower than the sensitivity of the replicase to said substitution. That is, the inventors researched and discovered that there is a first, lower threshold, in substitution of U to N1Ψ, which triggers evasion of the innate immune response thereby improving self-amplification of the SAM RNA or auto-amplification of the AAM RNA. The inventors also researched and discovered that there is also a direct effect of substitution on inhibiting self-amplification of the SAM RNA or the auto-amplification of the plurality of the AAM RNA. Fifty percent substitution results in lower double-stranded RNA levels than levels with 25% substitution, but these levels are higher than levels with 0% substitution. One hundred percent substitution results in almost no double-stranded RNA formation. There is therefore a second, higher, threshold between 50% and 100% substitution (from ˜75% substitution to ˜50% substitution) whereby the substitution directly inhibits said self-amplification or said auto-amplification. In combination, there is a balance whereby some substitution increases self-amplification by evading, or lowering, the innate immune response, but too much substitution inhibits induction of an immune response to the antigen and thereby the pathogen by inhibiting self-amplification of the SAM.

Said substitution within said ranges will suffice to provide an effective intracellular introduction of an heterologous nucleic acid, which may encode a heterologous protein such as an antigen to induce an immune response, or a protective immune response, to the antigen, and thereby the pathogen. Accordingly, the inventors researched and identified the following aspects and embodiments, as numerated:
1. A self-amplifying messenger (SAM) ribonucleic acid (RNA) comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.
2. The SAM RNA of numeral 1, the first mole percentage being to 70%.
3. The SAM RNA of numeral 1, the first mole percentage being to 65%.
4. The SAM RNA of numeral 1, the first mole percentage being to 60%.
5. The SAM RNA of numeral 1, the first mole percentage being to 55%.
6. The SAM RNA of numeral 1, the first mole percentage being to 50%.
7. The SAM RNA of any one of numerals 1-6, the first mole percentage being from 20%.
8. The SAM RNA of any one of numerals 1-6, the first mole percentage being from 25%.
9. The SAM RNA of any one of numerals 1-8, the one or more proteins capable of replicating the SAM RNA in an intracellular environment comprising an alphavirus non-structural protein-1 (nsP1), an alphavirus nsP2, an alphavirus nsP3, and an alphavirus nsP4.
10. The SAM RNA of any one of numerals 1-9, the heterologous nucleic acid encoding a heterologous protein.
11. The SAM RNA of any one of numerals 1-10, the heterologous nucleic acid comprising an inhibitory RNA.
12. The SAM RNA of numeral 11, the inhibitory RNA comprising an antisense RNA, a small interfering RNA, or a microRNA.
13. The SAM RNA of any one of numerals 10-12, the heterologous protein comprising an immunogen, an antibody, or an immunotherapeutic molecule.
14. The SAM RNA of any one of numerals 10-13, the heterologous protein comprising an immunogen or an antibody against the immunogen.
15. The SAM RNA of any one of numerals 1-14 further comprising a poly-adenosine monophosphate (poly(A)) tail.
16. The SAM RNA of any one of numerals 1-15 further comprising a 5′ untranslated region (5′ UTR), the 5′ UTR being 5′ of the first RNA segment and the second RNA segment.
17. The SAM RNA of any one of numerals 1-16 further comprising a 3′ untranslated region (3′ UTR), the 3′ UTR being 3′ of the first RNA segment and the second RNA segment, and optionally being 5′ of the poly(A) tail.
18. The SAM RNA of any one of numerals 1-17 further comprising a 5′ cap.
19. The SAM RNA of numeral 18, the 5′ cap being a cap-0, a cap-1, or a cap-2.
20. The SAM RNA of numeral 18, the 5′ cap being a cap-1.
21. The SAM RNA of numeral 18, the 5′ cap being a cap-0.
22. A composition comprising the SAM RNA of any one of numerals 1-21 and a pharmaceutically acceptable delivery vehicle.
23. The composition of numeral 22, the pharmaceutically acceptable delivery vehicle comprising a lipid nanoparticle (LNP).
24. The composition of numeral 23, the LNP encapsulating the SAM RNA.
25. A method of eliciting an immune response in a subject to the immunogen, the method comprising administering to the subject an effective amount of the SAM RNA of any one of numerals 13-21 or the composition of any one of numerals 22-24.
26. The method of numeral 25, the immune response being a protective immune response.
27. The method of numeral 25, the immune response being a therapeutic immune response.
28. The method of any one of numerals 25-27, the immunogen comprising a venom, a poison, an allergen, a cancer antigen, a bacterial antigen, a viral antigen, a fungal antigen, a parasite antigen, or a fragment thereof.
29. The method of any one of numerals 25-28, the heterologous protein comprising the antibody against the immunogen.
30. The method of any one of numerals 25-28, the heterologous protein comprising the immunogen.
31. A method of delivering the inhibitory RNA in the SAM RNA of any one of numerals 11-21 to a subject, the method comprising administering to the subject an effective amount of the SAM RNA.
32. A method of delivering to a subject the inhibitory RNA in the composition of any one of numerals 22-24, the method comprising administering to the subject an effective amount of the composition.
33. The method of numeral 31 or numeral 32, the inhibitory RNA being against a endogenous messenger RNA encoding a endogenous protein of the subject, and the administering decreasing the expression of the endogenous protein in the subject when compared to the expression of the endogenous protein in the subject without the administering.
34. A method of delivering to a subject the heterologous nucleic acid in the SAM RNA of any one of numerals 1-21, the method comprising administering an effective amount of the SAM RNA.
35. A method of delivering to a subject the heterologous nucleic acid in the composition of any one of numerals 22-24, the method comprising administering to the subject an effective amount of the composition.
36. The method of any one of numerals 25-36, the subject being human.
37. A method of manufacturing the SAM RNA of any one of numerals 1-17, the method comprising admixing an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the admixing being under conditions wherein the RNA polymerase produces the SAM RNA from the template nucleic acid.
38. A method of manufacturing the SAM RNA of any one of numerals 18-21, the method comprising:
a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the first admixing being under conditions wherein the RNA polymerase produces the SAM RNA from the template nucleic acid, thereby obtaining an uncapped SAM RNA; and
a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.
39. The method of numeral 37 or numeral 38, the RNA polymerase being a T7 RNA polymerase.
40. A method of manufacturing the composition of any one of numerals 22-24, the method comprising encapsulating the SAM RNA in the pharmaceutically acceptable delivery vehicle or adsorbing the SAM RNA to the pharmaceutically acceptable delivery vehicle.
41. A use of the SAM RNA of any one of numerals 1-21 for the manufacture of a medicament for delivering the heterologous nucleic acid, the use comprising admixing the SAM RNA with a pharmaceutically acceptable delivery vehicle.
42. A use of the SAM RNA of any one of numerals 14-21 for the manufacture of a medicament for preventing a disease caused by a pathogen; the pathogen comprising the immunogen; the use comprising admixing the SAM RNA with a pharmaceutically acceptable delivery vehicle.
43. A use of the SAM RNA of any one of numerals 14-21 for the manufacture of a medicament for treating a disease caused by a pathogen; the pathogen comprising the immunogen; the use comprising admixing the SAM RNA with a pharmaceutically acceptable delivery vehicle.
44. The use of any one of numerals 41-43, the pharmaceutically acceptable delivery vehicle comprising a lipid nanoparticle (LNP).
45. The use of numeral 44, the LNP encapsulating the SAM RNA.
46. The use of any one of numerals 42-45, the immunogen comprising a venom, a poison, an allergen, a cancer antigen, a bacterial antigen, a viral antigen, a fungal antigen, a parasite antigen, or a fragment thereof.
47. The use of any one of numerals 42-46, the heterologous protein comprising the antibody against the immunogen.
48. The use of any one of numerals 42-46, the heterologous protein comprising the immunogen.
49. A SAM RNA for use in eliciting an immune response to an antigen in a subject; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.
50. A SAM RNA for use in preventing infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.
51. A SAM RNA for use in treating infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.
52. A SAM RNA of any one of numerals 49-51, the subject being a human.
53. The SAM RNA of any one of numerals 49-52, the first mole percentage being to 70%.
54. The SAM RNA of any one of numerals 49-52, the first mole percentage being to 65%.
55. The SAM RNA of any one of numerals 49-52, the first mole percentage being to 60%.
56. The SAM RNA of any one of numerals 49-52, the first mole percentage being to 55%.
57. The SAM RNA of any one of numerals 49-52, the first mole percentage being to 50%.
58. The SAM RNA of any one of numerals 49-57, the first mole percentage being from 20%.
59. The SAM RNA of any one of numerals 49-57, the first mole percentage being from 25%.
60. The SAM RNA of any one of numerals 49-59, the one or more proteins capable of replicating the SAM RNA in an intracellular environment comprising an alphavirus non-structural protein-1 (nsP1), an alphavirus nsP2, an alphavirus nsP3, and an alphavirus nsP4.
61. The SAM RNA of any one of numerals 49-60 further comprising a poly-adenosine monophosphate (poly(A)) tail.
62. The SAM RNA of any one of numerals 49-61 further comprising a 5′ untranslated region (5′ UTR), the 5′ UTR being 5′ of the first RNA segment and the second RNA segment.
63. The SAM RNA of any one of numerals 49-61 further comprising a 3′ untranslated region (3′ UTR), the 3′ UTR being 3′ of the first RNA segment and the second RNA segment, and optionally being 5′ of the poly(A) tail.
64. The SAM RNA of any one of numerals 49-63 further comprising a 5′ cap.
65. The SAM RNA of numeral 64, the 5′ cap being a cap-0, a cap-1, or a cap-2.
66. The SAM RNA of numeral 64, the 5′ cap being a cap-1.
67. The SAM RNA of numeral 64, the 5′ cap being a cap-0.
68. A composition comprising the SAM RNA of any one of numerals 49-67 and a pharmaceutically acceptable delivery vehicle.
69. The composition of numeral 68, the pharmaceutically acceptable delivery vehicle comprising a lipid nanoparticle (LNP).
70. The composition of numeral 69, the LNP encapsulating the SAM RNA.
71. A method of manufacturing the SAM RNA of any one of numerals 49-63, the method comprising admixing an RNA polymerase, the N1-methylpseudouridines, the uridines, and a template nucleic acid comprising a sequence of the SAM RNA, thereby obtaining an admixture; the admixture comprising a second mole percentage of the N1-methylpseudouridines to the total of the N1-methylpseudouridines and the uridines being the same as the first mole percentage; the admixing being under conditions wherein the RNA polymerase produces the SAM RNA from the template nucleic acid.
72. A method of manufacturing the SAM RNA of any one of numerals 64-67, the method comprising:
a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the first admixing being under conditions wherein the RNA polymerase produces the SAM RNA from the template nucleic acid, thereby obtaining an uncapped SAM RNA; and
a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.
73. The method of numeral 71 or numeral 72, the RNA polymerase being a T7 RNA polymerase.
74. A method of manufacturing the composition of any one of numerals 68-70, the method comprising encapsulating the SAM RNA in the pharmaceutically acceptable delivery vehicle or adsorbing the SAM RNA to the pharmaceutically acceptable delivery vehicle.
75. A SAM RNA for use in delivering a heterologous nucleic acid to a subject, the SAM RNA comprising N1-methylpseudorudines, uridines, a RNA segment that comprises a heterologous nucleic acid, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.
76. The SAM RNA of numeral 75, the subject being a human.
77. The SAM RNA of numeral 75 or numeral 76, the first mole percentage being to 70%.
78. The SAM RNA of numeral 75 or numeral 76, the first mole percentage being to 65%.
79. The SAM RNA of numeral 75 or numeral 76, the first mole percentage being to 60%.
80. The SAM RNA of numeral 75 or numeral 76, the first mole percentage being to 55%.
81. The SAM RNA of numeral 75 or numeral 76, the first mole percentage being to 50%.
82. The SAM RNA of any one of numerals 75-81, the first mole percentage being from 20%.
83. The SAM RNA of any one of numerals 75-81, the first mole percentage being from 25%.
84. The SAM RNA of any one of numerals 75-83, the one or more proteins capable of replicating the SAM RNA in an intracellular environment comprising an alphavirus non-structural protein-1 (nsP1), an alphavirus nsP2, an alphavirus nsP3, and an alphavirus nsP4.
85. The SAM RNA of any one of numerals 75-84 further comprising a poly-adenosine monophosphate (poly(A)) tail.
86. The SAM RNA of any one of numerals 75-85 further comprising a 5′ untranslated region (5′ UTR), the 5′ UTR being 5′ of the first RNA segment and the second RNA segment.
87. The SAM RNA of any one of numerals 75-86 further comprising a 3′ untranslated region (3′ UTR), the 3′ UTR being 3′ of the first RNA segment and the second RNA segment, and optionally being 5′ of the poly(A) tail.
88. The SAM RNA of any one of numerals 75-87, the heterologous nucleic acid encoding a heterologous protein.
89. The SAM RNA of any one of numerals 75-88, the heterologous nucleic acid comprising an inhibitory RNA.
90. The SAM RNA of numeral 89, the inhibitory RNA comprising an antisense RNA, a small interfering RNA, or a microRNA.
91. The SAM RNA of any one of numerals 88-90, the heterologous protein comprising an immunogen, an antibody, an antibody against the immunogen, an immunotherapeutic molecule, or an antibody against an immune signaling molecule.
92. The SAM RNA of any one of numerals 88-90, the heterologous protein comprising an immunogen or an antibody against the immunogen.
93. The SAM RNA of any one of numerals 75-92 further comprising a 5′ cap.
94. The SAM RNA of numeral 93, the 5′ cap being a cap-0, a cap-1, or a cap-2.
95. The SAM RNA of numeral 93, the 5′ cap being a cap-1.
96. The SAM RNA of numeral 93, the 5′ cap being a cap-0.
97. A composition comprising the SAM RNA of any one of numerals 75-96 and a pharmaceutically acceptable delivery vehicle.
98. The composition of numeral 97, the pharmaceutically acceptable delivery vehicle comprising a lipid nanoparticle (LNP).
99. The composition of numeral 98, the LNP encapsulating the SAM RNA.
100. A SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.
101. The SAM RNA of numeral 100, the first mole percentage being to 70%.
102. The SAM RNA of numeral 100, the first mole percentage being to 65%.
103. The SAM RNA of numeral 100, the first mole percentage being to 60%.
104. The SAM RNA of numeral 100, the first mole percentage being to 55%.
105. The SAM RNA of numeral 100, the first mole percentage being to 50%.
106. The SAM RNA of any one of numerals 100-105, the first mole percentage being from 20%.
107. The SAM RNA of any one of numerals 100-105, the first mole percentage being from 25%.
108. The SAM RNA of any one of numerals 100-107, the one or more proteins capable of replicating the SAM RNA in an intracellular environment comprising an alphavirus non-structural protein-1 (nsP1), an alphavirus nsP2, an alphavirus nsP3, and an alphavirus nsP4.
109. The SAM RNA of any one of numerals 100-108, the heterologous nucleic acid encoding a heterologous protein.
110. The SAM RNA of any one of numerals 100-109, the heterologous nucleic acid comprising an inhibitory RNA.
111. The SAM RNA of numeral 110, the inhibitory RNA comprising an antisense RNA, a small interfering RNA, or a microRNA.
112. The SAM RNA of any one of numerals 109-111, the heterologous protein comprising an immunogen, an antibody, an antibody against the immunogen, an immunotherapeutic molecule, or an antibody against an immune signaling molecule.
113. The SAM RNA of any one of numerals 109-112, the heterologous protein comprising an immunogen or an antibody against the immunogen.
114. The SAM RNA of any one of numerals 100-113 further comprising a poly-adenosine monophosphate (poly(A)) tail.
115. The SAM RNA of any one of numerals 100-114 further comprising a 5′ untranslated region (5′ UTR), the 5′ UTR being 5′ of the first RNA segment and the second RNA segment.
116. The SAM RNA of any one of numerals 100-114 further comprising a 3′ untranslated region (3′ UTR), the 3′ UTR being 3′ of the first RNA segment and the second RNA segment, and optionally being 5′ of the poly(A) tail.
117. The SAM RNA of any one of numerals 100-116 further comprising a 5′ cap.
118. The SAM RNA of numeral 117, the 5′ cap being a cap-0, a cap-1, or a cap-2.
119. The SAM RNA of numeral 117, the 5′ cap being a cap-1.
120. The SAM RNA of numeral 117, the 5′ cap being a cap-0.
121. A composition comprising the SAM RNA of any one of numerals 100-120 and a pharmaceutically acceptable delivery vehicle.
122. The composition of numeral 121, the pharmaceutically acceptable delivery vehicle comprising a lipid nanoparticle (LNP).
123. The composition of numeral 122, the LNP encapsulating the SAM RNA.
124. A method of eliciting an immune response in a subject to the immunogen, the method comprising administering to the subject an effective amount of the SAM RNA of any one of numerals 113-120 or the composition of any one of numerals 121-123.
125. The method of numeral 124, the immune response being a protective immune response.
126. The method of numeral 124, the immune response being a therapeutic immune response.
127. The method of any one of numerals 124-126, the immunogen comprising a venom, a poison, an allergen, a cancer antigen, a bacterial antigen, a viral antigen, a fungal antigen, a parasite antigen, or a fragment thereof.
128. The method of numeral of any one of numerals 124-127, the heterologous protein comprising the antibody against the immunogen.
129. The method of any one of numerals 124-127, the heterologous protein comprising the immunogen.
130. A method of delivering the inhibitory RNA in the SAM RNA of any one of numerals 110-121 to a subject, the method comprising administering to the subject an effective amount of the SAM RNA.
131. A method of delivering to a subject the inhibitory RNA in the composition of any one of numerals 121-123, the method comprising administering to the subject an effective amount of the composition.
132. The method of numeral 130 or numeral 131, the inhibitory RNA being against a endogenous messenger RNA encoding a endogenous protein of the subject, and the administering decreasing the expression of the endogenous protein in the subject when compared to the expression of the endogenous protein in the subject without the administering.
133. A method of delivering to a subject the heterologous nucleic acid in the SAM RNA of any one of numerals 100-120, the method comprising administering an effective amount of the SAM RNA.
134. A method of delivering to a subject the heterologous nucleic acid in the composition of any one of numerals 121-123, the method comprising administering to the subject an effective amount of the composition.
135. A method of manufacturing the composition of any one of numerals 121-123 comprising encapsulating the SAM RNA in the pharmaceutically acceptable delivery vehicle or adsorbing the SAM RNA to the pharmaceutically acceptable delivery vehicle.
136. A use of the SAM RNA of any one of numerals 100-120 for the manufacture of a medicament for delivering the heterologous nucleic acid, the use comprising admixing the SAM RNA with a pharmaceutically acceptable delivery vehicle.
137. A use of the SAM RNA of any one of numerals 113-120 for the manufacture of a medicament for preventing a disease caused by a pathogen; the pathogen comprising the immunogen; the use comprising admixing the SAM RNA with a pharmaceutically acceptable delivery vehicle.
138. A use of the SAM RNA of any one of numerals 113-120 for the manufacture of a medicament for treating a disease caused by a pathogen; the pathogen comprising the immunogen; the use comprising admixing the SAM RNA with a pharmaceutically acceptable delivery vehicle.
139. The use of any one of numerals 136-138, the pharmaceutically acceptable delivery vehicle comprising a lipid nanoparticle (LNP).
140. The use of numeral 139, the LNP encapsulating the SAM RNA.
141. The use of any one of numerals 137-140, the immunogen comprising a venom, a poison, an allergen, a cancer antigen, a bacterial antigen, a viral antigen, a fungal antigen, a parasite antigen, or a fragment thereof.
142. The use of any one of numerals 137-141, the heterologous protein comprising the antibody against the immunogen.
143. The of any one of numerals 137-141, the heterologous protein comprising the immunogen.
144. A SAM RNA for use in eliciting an immune response to an antigen in a subject; the SAM RNA comprising N1-methylpseudouridines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of N1-methylpseudouridines to the total of the N1-methylpseudouridines and the uridines from 15% to 75%; the admixing being under conditions wherein the RNA polymerase produces the SAM RNA from the template nucleic acid.
145. A SAM RNA for use in preventing infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of N1-methylpseudouridines to the total of the N1-methylpseudouridines and the uridines from 15% to 75%; the admixing being under conditions wherein the RNA polymerase produces the SAM RNA from the template nucleic acid.
146. A SAM RNA for use in treating infection by a pathogen in a subject; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.
147. The SAM RNA of any one of numerals 144-146, the subject being a human.
148. The SAM RNA of any one of numerals 144-147, the first mole percentage being to 70%.
149. The SAM RNA of any one of numerals 144-147, the first mole percentage being to 65%.
150. The SAM RNA of any one of numerals 144-147, the first mole percentage being to 60%.
151. The SAM RNA of any one of numerals 144-147, the first mole percentage being to 55%.
152. The SAM RNA of any one of numerals 144-147, the first mole percentage being to 50%.
153. The SAM RNA of any one of numerals 144-152, the first mole percentage being from 20%.
154. The SAM RNA of any one of numerals 144-152, the first mole percentage being from 25%.
155. The SAM RNA of any one of numerals 144-154, the one or more proteins capable of replicating the SAM RNA in an intracellular environment comprising an alphavirus non-structural protein-1 (nsP1), an alphavirus nsP2, an alphavirus nsP3, and an alphavirus nsP4.
156. The SAM RNA of any one of numerals 144-155 further comprising a poly-adenosine monophosphate (poly(A)) tail.
157. The SAM RNA of any one of numerals 144-156 further comprising a 5′ untranslated region (5′ UTR), the 5′ UTR being 5′ of the first RNA segment and the second RNA segment.
158. The SAM RNA of any one of numerals 144-157 further comprising a 3′ untranslated region (3′ UTR), the 3′ UTR being 3′ of the first RNA segment and the second RNA segment, and optionally being 5′ of the poly(A) tail.
159. The SAM RNA of any one of numerals 144-154 further comprising a 5′ cap.
160. The SAM RNA of numeral 159, the 5′ cap being a cap-0, a cap-1, or a cap-2.
161. The SAM RNA of numeral 159, the 5′ cap being a cap-1.
162. The SAM RNA of numeral 159, the 5′ cap being a cap-0.
163. The SAM RNA of any one of numerals 144-162, the RNA polymerase being a T7 RNA polymerase.
164. A composition comprising the SAM RNA of any one of numerals 144-163 and a pharmaceutically acceptable delivery vehicle.
165. The composition of numeral 164, the pharmaceutically acceptable delivery vehicle comprising a lipid nanoparticle (LNP).
166. The composition of numeral 165, the LNP encapsulating the SAM RNA.
167. A method of manufacturing the composition of any one of numerals 164-166, the method comprising encapsulating the SAM RNA in the pharmaceutically acceptable delivery vehicle or adsorbing the SAM RNA to the pharmaceutically acceptable delivery vehicle.
168. A SAM RNA for use in delivering a heterologous nucleic acid to a subject, the SAM RNA comprising N1-methylpseudorudines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.
169. The SAM RNA of numeral 168, the subject being a human.
170. The SAM RNA of numeral 168 or numeral 169, the first mole percentage being to 70%.
171. The SAM RNA of numeral 168 or numeral 169, the first mole percentage being to 65%.
172. The SAM RNA of numeral 168 or numeral 169, the first mole percentage being to 60%.
173. The SAM RNA of numeral 168 or numeral 169, the first mole percentage being to 55%.
174. The SAM RNA of numeral 168 or numeral 169, the first mole percentage being to 50%.
175. The SAM RNA of any one of numerals 168-174, the first mole percentage being from 20%.
176. The SAM RNA of any one of numerals 168-174, the first mole percentage being from 25%.
177. The SAM RNA of any one of numerals 168-176, the one or more proteins capable of replicating the SAM RNA in an intracellular environment comprising an alphavirus non-structural protein-1 (nsP1), an alphavirus nsP2, an alphavirus nsP3, and an alphavirus nsP4.
178. The SAM RNA of any one of numerals 168-177 further comprising a poly-adenosine monophosphate (poly(A)) tail.
179. The SAM RNA of any one of numerals 168-178 further comprising a 5′ untranslated region (5′ UTR), the 5′ UTR being 5′ of the first RNA segment and the second RNA segment.
180. The SAM RNA of any one of numerals 168-179 further comprising a 3′ untranslated region (3′ UTR), the 3′ UTR being 3′ of the first RNA segment and the second RNA segment, and optionally being 5′ of the poly(A) tail.
181. The SAM RNA of any one of numerals 168-180, the heterologous nucleic acid encoding a heterologous protein.
182. The SAM RNA of any one of numerals 168-181, the heterologous nucleic acid comprising an inhibitory RNA.
183. The SAM RNA of numeral 182, the inhibitory RNA comprising an antisense RNA, a small interfering RNA, or a microRNA.
184. The SAM RNA of any one of numerals 181-183, the heterologous protein comprising an immunogen, an antibody against the immunogen, an immunotherapeutic molecule, or an antibody against an immune signaling molecule.
185. The SAM RNA of any one of numerals 181-183, the heterologous protein comprising an immunogen or an antibody against the immunogen.
186. The SAM RNA of any one of numerals 168-185 further comprising a 5′ cap.
187. The SAM RNA of numeral 186, the 5′ cap being a cap-0, a cap-1, or a cap-2.
188. The SAM RNA of numeral 186, the 5′ cap being a cap-1.
189. The SAM RNA of numeral 186, the 5′ cap being a cap-0.
190. A composition comprising the SAM RNA of any one of numerals 168-189 and a pharmaceutically acceptable delivery vehicle.
191. The composition of numeral 190, the pharmaceutically acceptable delivery vehicle comprising a lipid nanoparticle (LNP).
192. The composition of numeral 191, the LNP encapsulating the SAM RNA.
193. A plurality of auto-amplifying messenger (AAM) RNAs comprising N1-methylpseudouridines, uridines, a first RNA, and one or more second RNA; the first RNA comprising a heterologous nucleic acid; the one or more second RNA encoding one or more proteins capable of replicating the SAM RNA in an intracellular environment; the plurality of AAM RNAs having a first mole percentage of the N1-methylpseudouridines to the total of the N1-methypseudouridines and uridines from 15% to 75%.
194. The plurality of AAM RNAs of numeral 193, the first RNA comprising the N1-methylpseudouridines and the uridines.
195. The plurality of AAM RNAs of numeral 193 or numeral 194, the one or more second RNA comprising the N1-methylpseudouridines and the uridines.
196. The plurality of AAM RNAs of any one of numerals 193-195, the first mole percentage being to 70%.
197. The plurality of AAM RNAs of any one of numerals 193-195, the first mole percentage being to 65%.
198. The plurality of AAM RNAs of any one of numerals 193-195, the first mole percentage being to 60%.
199. The plurality of AAM RNAs of any one of numerals 193-195, the first mole percentage being to 55%.
200. The plurality of AAM RNAs of any one of numerals 193-195, the first mole percentage being to 50%.
201. The plurality of AAM RNAs of any one of numerals 193-200, the first mole percentage being from 20%.
202. The plurality of AAM RNAs of any one of numerals 193-200, the first mole percentage being from 25%.
203. The plurality of AAM RNAs of any one of numerals 193-202, the one or more proteins that together are capable of replicating the plurality of AAM RNAs in an intracellular environment comprising an alphavirus non-structural protein-1 (nsP1), an alphavirus nsP2, an alphavirus nsP3, and an alphavirus nsP4.
204. The plurality of AAM RNAs of any one of numerals 193-203, the heterologous nucleic acid encoding a heterologous protein.
205. The plurality of AAM RNAs of any one of numerals 193-204, the heterologous nucleic acid comprising an inhibitory RNA.
206. The plurality of AAM RNAs of numeral 205, the inhibitory RNA comprising an antisense RNA, a small interfering RNA, or a microRNA.
207. The plurality of AAM RNAs of any one of numerals 193-206, the heterologous protein comprising an immunogen, an antibody, or an immunotherapeutic molecule.
208. The plurality of AAM RNAs of any one of numerals 193-207, the heterologous protein comprising an immunogen or an antibody against the immunogen.
209. The plurality of AAM RNAs of any one of numerals 193-208 further comprising a poly-adenosine monophosphate (poly(A)) tail.
210. The plurality of AAM RNAs of any one of numerals 193-209 further comprising a 5′ untranslated region (5′ UTR), the 5′ UTR being 5′ of the first RNA or the second RNA.
211. The plurality of AAM RNAs of any one of numerals 193-210 further comprising a 3′ untranslated region (3′ UTR), the 3′ UTR being 3′ of the first RNA or the second RNA, and optionally being 5′ of the poly(A) tail.
212. The plurality of AAM RNAs of any one of numerals 193-211 further comprising a 5′ cap.
213. The plurality of AAM RNAs of numeral 212, the 5′ cap being a cap-0, a cap-1, or a cap-2.
214. The plurality of AAM RNAs of numeral 212, the 5′ cap being a cap-1.
215. The plurality of AAM RNAs of numeral 212, the 5′ cap being a cap-0.
216. A composition comprising the plurality of AAM RNAs of any one of numerals 193-215 and a pharmaceutically acceptable delivery vehicle.
217. The composition of numeral 216, the pharmaceutically acceptable delivery vehicle comprising a lipid nanoparticle (LNP).
218. The composition of numeral 217, the LNP encapsulating at least one of the one or more first RNA or at least one of the one.
219. A method of eliciting an immune response in a subject to the immunogen, the method comprising administering to the subject an effective amount of the plurality of AAM RNAs of any one of numerals 208-215 or the composition of any one of numerals 216-218.
220. The method of numeral 219, the immune response being a protective immune response.
221. The method of numeral 219, the immune response being a therapeutic immune response.
222. The method of any one of numerals 219-221, the immunogen comprising a venom, a poison, an allergen, a cancer antigen, a bacterial antigen, a viral antigen, a fungal antigen, a parasite antigen, or a fragment thereof.
223. The method of any one of numerals 219-222, the heterologous protein comprising the antibody against the immunogen.
224. The method of any one of numerals 219-222, the heterologous protein comprising the immunogen.
225. A method of delivering the inhibitory RNA in the plurality of AAM RNAs of any one of numerals 205-215 to a subject, the method comprising administering to the subject an effective amount of the plurality of AAM RNAs.
226. A method of delivering to a subject the inhibitory RNA in the composition of any one of numerals 216-218, the method comprising administering to the subject an effective amount of the composition.
227. The method of numeral 225 or numeral 226, the inhibitory RNA being against a endogenous messenger RNA encoding a endogenous protein of the subject, and the administering decreasing the expression of the endogenous protein in the subject when compared to the expression of the endogenous protein in the subject without the administering.
228. A method of delivering to a subject the heterologous nucleic acid in the plurality of AAM RNAs of any one of numerals 193-215, the method comprising administering an effective amount of the plurality of AAM RNAs.
229. A method of delivering to a subject the heterologous nucleic acid in the composition of any one of numerals 216-218, the method comprising administering to the subject an effective amount of the composition.
230. The method of any one of numerals 219-228, wherein the subject is human.
231. A method of manufacturing the plurality of AAM RNAs of any one of numerals 193-211, the method comprising one or more admixing of an RNA polymerase, the N1-methylpseudouridines, the uridines, and one or more template nucleic acids, thereby obtaining an admixture wherein the one or more template nucleic acids comprise a sequence of the plurality of AAM RNAs; the one or more admixture having a second mole percentage of the N1-methylpseudouridines to the total of the N1-methylpseudouridines and the uridines; the second mole percentage being the same as the first mole percentage; the one or more admixing being under conditions wherein the RNA polymerase produces the plurality of AAM RNAs from the one or more template nucleic acids.
232. A method of manufacturing the plurality of AAM RNAs of any one of numerals 212-215, the method comprising:
one or more first admixing of an RNA polymerase, the N1-methylpseudouridines, the uridines, and one or more template nucleic acids, thereby obtaining one or more first admixtures wherein the one or more template nucleic acids comprise a sequence of the plurality of AAM RNAs; the one or more first admixture having a second mole percentage of the N1-methylpseudouridines to the total of the N1-methylpseudouridines and the uridines; the second mole percentage being the same as the first mole percentage; the one or more first admixing being under conditions wherein the RNA polymerase produces a plurality of uncapped AAM RNAs from the one or more template nucleic acids, wherein the plurality of uncapped AAM RNA have the sequence of the plurality of the AAM RNA; and a one or more second admixing of the plurality of the uncapped AAM RNAs, a messenger RNA guanylyltransferase, guanosine triphosphate, a (guaninine-N7-)-methyltransferase, and S-adenosyl-L-methionine, the one or more second admixing being under conditions that cause a 5′ to 5′ triphosphate cap linkage, the one or more second admixing optionally further comprising a 2′-O-methyltransferase, and optionally the one or more second admixing being under conditions that form cap-1 or cap-2.
233. The method of numeral 232 or numeral 233, the RNA polymerase being a T7 RNA polymerase.
234. A method of manufacturing the composition of any one of numerals 216-218, the method comprising encapsulating the plurality of AAM RNAs in the pharmaceutically acceptable delivery vehicle or adsorbing the plurality of AAM RNAs to the pharmaceutically acceptable delivery vehicle.
235. A use of the plurality of AAM RNAs of any one of numerals 193-215 for the manufacture of a medicament for delivering the heterologous nucleic acid, the use comprising admixing the plurality of AAM RNAs with a pharmaceutically acceptable delivery vehicle.
236. A use of the plurality of AAM RNAs of any one of numerals 208-215 for the manufacture of a medicament for preventing a disease caused by a pathogen; the pathogen comprising the immunogen; the use comprising admixing the plurality of AAM RNAs with a pharmaceutically acceptable delivery vehicle.
237. A use of the plurality of AAM RNAs of any one of numerals 208-215 for the manufacture of a medicament for treating a disease caused by a pathogen; the pathogen comprising the immunogen; the use comprising admixing the plurality of AAM RNAs with a pharmaceutically acceptable delivery vehicle.
238. The use of any one of numerals 235-237, the pharmaceutically acceptable delivery vehicle comprising a lipid nanoparticle (LNP).
239. The use of numeral 238, the LNP encapsulating the plurality of AAM RNAs.
240. The use of any one of numerals 236-239, the immunogen comprising a venom, a poison, an allergen, a cancer antigen, a bacterial antigen, a viral antigen, a fungal antigen, a parasite antigen, or a fragment thereof.
241. The use of any one of numerals 236-240, the heterologous protein comprising the antibody against the immunogen.
242. The of any one of numerals 236-240, the heterologous protein comprising the immunogen.
243. A plurality of AAM RNAs for use in eliciting an immune response to an antigen in a subject; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.
244. A plurality of AAM RNAs for use in preventing infection by a pathogen in a subject; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.
245. A plurality of AAM RNAs for use in treating infection by a pathogen in a subject; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.
246. A plurality of AAM RNAs of any one of numerals 243-245, the subject being a human.
247. The plurality of AAM RNAs of any one of numerals 243-246, the first RNA comprising the N1-methylpseudouridines and the uridines.
248. The plurality of AAM RNAs of any one of numerals 243-247, the one or more second RNA comprising the N1-methylpseudouridines and the uridines.
249. The plurality of AAM RNAs of any one of numerals 243-248, the first mole percentage being to 70%.
250. The plurality of AAM RNAs of any one of numerals 243-248, the first mole percentage being to 65%.
251. The plurality of AAM RNAs of any one of numerals 243-248, the first mole percentage being to 60%.
252. The plurality of AAM RNAs of any one of numerals 243-248, the first mole percentage being to 55%.
253. The plurality of AAM RNAs of any one of numerals 243-248, the first mole percentage being to 50%.
254. The plurality of AAM RNAs of any one of numerals 243-253, the first mole percentage being from 20%.
255. The plurality of AAM RNAs of any one of numerals 243-253, the first mole percentage being from 25%.
256. The plurality of AAM RNAs of any one of numerals 243-255, the one or more proteins capable of replicating the plurality of AAM RNAs in an intracellular environment comprising an alphavirus non-structural protein-1 (nsP1), an alphavirus nsP2, an alphavirus nsP3, and an alphavirus nsP4.
257. The plurality of AAM RNAs of any one of numerals 243-256 further comprising a poly-adenosine monophosphate (poly(A)) tail.
258. The plurality of AAM RNAs of any one of numerals 243-257 further comprising a 5′ untranslated region (5′ UTR), the 5′ UTR being 5′ of the first RNA or the second RNA.
259. The plurality of AAM RNAs of any one of numerals 243-258 further comprising a 3′ untranslated region (3′ UTR), the 3′ UTR being 3′ of the first RNA or the second RNA, and optionally being 5′ of the poly(A) tail.
260. The plurality of AAM RNAs of any one of numerals 243-259 further comprising a 5′ cap.
261. The plurality of AAM RNAs of numeral 260, the 5′ cap being a cap-0, a cap-1, or a cap-2.
262. The plurality of AAM RNAs of numeral 260, the 5′ cap being a cap-1.
263. The plurality of AAM RNAs of numeral 260, the 5′ cap being a cap-0.
264. A composition comprising the plurality of AAM RNAs of any one of numerals 243-263 and a pharmaceutically acceptable delivery vehicle.
265. The composition of numeral 264, the pharmaceutically acceptable delivery vehicle comprising a lipid nanoparticle (LNP).
266. The composition of numeral 264, the LNP encapsulating the plurality of AAM RNAs.
267. A method of manufacturing the plurality of AAM RNAs of any one of numerals 243-260, the method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a second mole percentage of the N1-methylpseudouridines to the total of the N1-methylpseudouridines and the uridines; the second mole percentage being the same as the first mole percentage; the one or more admixing being under conditions wherein the RNA polymerase produces the plurality of AAM RNAs from the one or more template nucleic acids.
268. A method of manufacturing the plurality of AAM RNAs of any one of numerals 260-263, the method comprising:
one or more first admixing of an RNA polymerase, the N1-methylpseudouridines, the uridines, and one or more template nucleic acids, thereby obtaining one or more first admixtures wherein the one or more template nucleic acids comprise a sequence of the plurality of AAM RNAs; the one or more first admixtures having a second mole percentage of the N1-methylpseudouridines to the total of the N1-methylpseudouridines and the uridines; the second mole percentage being the same as the first mole percentage; the one or more first admixing being under conditions wherein the RNA polymerase produces a plurality of uncapped AAM RNAs from the one or more template nucleic acids, wherein the plurality of uncapped AAM RNA have the sequence of the plurality of the AAM RNA; and one or more second admixing of the plurality of uncapped AAM RNAs, a messenger RNA guanylyltransferase, guanosine triphosphate, a (guaninine-N7-)-methyltransferase, and S-adenosyl-L-methionine, the one or more second admixing being under conditions that cause a 5′ to 5′ triphosphate cap linkage, the one or more second admixing optionally further comprising a 2′-O-methyltransferase, and optionally the one or more second admixing being under conditions that form cap-1 or cap-2.
269. The method of numeral 267 or numeral 268, the RNA polymerase being a T7 RNA polymerase.
270. A method of manufacturing the composition of any one of numerals 264-267, the method comprising encapsulating the plurality of AAM RNAs in the pharmaceutically acceptable delivery vehicle or adsorbing the plurality of AAM RNAs to the pharmaceutically acceptable delivery vehicle.
271. A plurality of AAM RNAs for use in delivering a heterologous nucleic acid to a subject, the plurality of AAM RNAs comprising N1-methylpseudorudines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.
272. The plurality of AAM RNAs of numeral 271, the subject being a human.
273. The plurality of AAM RNAs of numeral 271 or numeral 272, the first RNA comprising the N1-methylpseudouridines and the uridines.
274. The plurality of AAM RNAs of any one of numerals 271-273, the one or more second RNA comprising the N1-methylpseudouridines and the uridines.
275. The plurality of AAM RNAs of any one of numerals 271-274, the first mole percentage being to 70%.
276. The plurality of AAM RNAs of any one of numerals 271-274, the first mole percentage being to 65%.
277. The plurality of AAM RNAs of any one of numerals 271-274, the first mole percentage being to 60%.
278. The plurality of AAM RNAs of any one of numerals 271-274, the first mole percentage being to 55%.
279. The plurality of AAM RNAs of any one of numerals 271-274, the first mole percentage being to 50%.
280. The plurality of AAM RNAs of any one of numerals 271-279, the first mole percentage being from 20%.
281. The plurality of AAM RNAs of any one of numerals 271-279, the first mole percentage being from 25%.
282. The plurality of AAM RNAs of any one of numerals 271-281, the one or more proteins capable of replicating the plurality of AAM RNAs in an intracellular environment comprising an alphavirus non-structural protein-1 (nsP1), an alphavirus nsP2, an alphavirus nsP3, and an alphavirus nsP4.
283. The plurality of AAM RNAs of any one of numerals 271-282 further comprising a poly-adenosine monophosphate (poly(A)) tail.
284. The plurality of AAM RNAs of any one of numerals 271-283 further comprising a 5′ untranslated region (5′ UTR), the 5′ UTR being 5′ of the first RNA or the second RNA.
285. The plurality of AAM RNAs of any one of numerals 271-284 further comprising a 3′ untranslated region (3′ UTR), the 3′ UTR being 3′ of the first RNA or the second RNA, and optionally being 5′ of the poly(A) tail.
286. The plurality of AAM RNAs of any one of numerals 271-285, the heterologous nucleic acid encoding a heterologous protein.
287. The plurality of AAM RNAs of any one of numerals 271-286, the heterologous nucleic acid comprising an inhibitory RNA.
288. The plurality of AAM RNAs of numeral 287, the inhibitory RNA comprising an antisense RNA, a small interfering RNA, or a microRNA.
289. The plurality of AAM RNAs of any one of numerals 286-288, the heterologous protein comprising an immunogen, an antibody, an antibody against the immunogen, an immunotherapeutic molecule, or an antibody against an immune signaling molecule.
290. The plurality of AAM RNAs of any one of numerals 286-288, the heterologous protein comprising an immunogen or an antibody against the immunogen.
291. The plurality of AAM RNAs of any one of numerals 271-290 further comprising a 5′ cap.
292. The plurality of AAM RNAs of numeral 291, the 5′ cap being a cap-0, a cap-1, or a cap-2.
293. The plurality of AAM RNAs of numeral 291, the 5′ cap being a cap-1.
294. The plurality of AAM RNAs of numeral 291, the 5′ cap being a cap-0.
295. A composition comprising the plurality of AAM RNAs of any one of numerals 271-294 and a pharmaceutically acceptable delivery vehicle.
296. The composition of numeral 295, the pharmaceutically acceptable delivery vehicle comprising a lipid nanoparticle (LNP).
297. The composition of numeral 296, the LNP encapsulating the plurality of AAM RNAs.
298. A plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.
299. The plurality of AAM RNAs of numeral 298, the first RNA comprising the N1-methylpseudouridines and the uridines.
300. The plurality of AAM RNAs of numeral 298 or numeral 299, the one or more second RNA comprising the N1-methylpseudouridines and the uridines.
301. The plurality of AAM RNAs of any one of numerals 298-300, the first mole percentage being to 70%.
302. The plurality of AAM RNAs of any one of numerals 298-300, the first mole percentage being to 65%.
303. The plurality of AAM RNAs of any one of numerals 298-300, the first mole percentage being to 60%.
304. The plurality of AAM RNAs of any one of numerals 298-300, the first mole percentage being to 55%.
305. The plurality of AAM RNAs of any one of numerals 298-300, the first mole percentage being to 50%.
306. The plurality of AAM RNAs of any one of numerals 298-305, the first mole percentage being from 20%.
307. The plurality of AAM RNAs of any one of numerals 298-305, the first mole percentage being from 25%.
308. The plurality of AAM RNAs of any one of numerals 298-307, the one or more proteins capable of replicating the plurality of AAM RNAs in an intracellular environment comprising an alphavirus non-structural protein-1 (nsP1), an alphavirus nsP2, an alphavirus nsP3, and an alphavirus nsP4.
309. The plurality of AAM RNAs of any one of numerals 298-308, the heterologous nucleic acid encoding a heterologous protein.
310. The plurality of AAM RNAs of any one of numerals 298-309, the heterologous nucleic acid comprising an inhibitory RNA.
311. The plurality of AAM RNAs of numeral 310, the inhibitory RNA comprising an antisense RNA, a small interfering RNA, or a microRNA.
312. The plurality of AAM RNAs of any one of numerals 309-311, the heterologous protein comprising an immunogen, an antibody, an antibody against the immunogen, an immunotherapeutic molecule, or an antibody against an immune signaling molecule.
313. The plurality of AAM RNAs of any one of numerals 309-311, the heterologous protein comprising an immunogen or an antibody against the immunogen.
314. The plurality of AAM RNAs of any one of numerals 298-313 further comprising a poly-adenosine monophosphate (poly(A)) tail.
315. The plurality of AAM RNAs of any one of numerals 298-314 further comprising a 5′ untranslated region (5′ UTR), the 5′ UTR being 5′ of the first RNA or the second RNA.
316. The plurality of AAM RNAs of any one of numerals 298-315 further comprising a 3′ untranslated region (3′ UTR), the 3′ UTR being 3′ of the first RNA or the second RNA, and optionally being 5′ of the poly(A) tail.
317. The plurality of AAM RNAs of any one of numerals 298-316 further comprising a 5′ cap.
318. The plurality of AAM RNAs of numeral 317, the 5′ cap being a cap-0, a cap-1, or a cap-2.
319. The plurality of AAM RNAs of numeral 317, the 5′ cap being a cap-1.
320. The plurality of AAM RNAs of numeral 317, the 5′ cap being a cap-0.
321. A composition comprising the plurality of AAM RNAs of any one of numerals 298-320 and a pharmaceutically acceptable delivery vehicle.
322. The composition of numeral 321, the pharmaceutically acceptable delivery vehicle comprising a lipid nanoparticle (LNP).
323. The composition of numeral 322, the LNP encapsulating the plurality of AAM RNAs.
324. A method of eliciting an immune response in a subject to the immunogen, the method comprising administering to the subject an effective amount of the plurality of AAM RNAs of any one of numerals 298-320 or the composition of any one of numerals 321-323.
325. The method of numeral 324, the immune response being a protective immune response.
326. The method of numeral 324, the immune response being a therapeutic immune response.
327. The method of any one of numerals 324-326, the immunogen comprising a venom, a poison, an allergen, a cancer antigen, a bacterial antigen, a viral antigen, a fungal antigen, a parasite antigen, or a fragment thereof.
328. The method of numeral of any one of numerals 324-327, the heterologous protein comprising the antibody against the immunogen.
329. The method of any one of numerals 324-327, the heterologous protein comprising the immunogen.
330. A method of delivering the inhibitory RNA in the plurality of AAM RNAs of any one of numerals 310-320 to a subject, the method comprising administering to the subject an effective amount of the plurality of AAM RNAs.
331. A method of delivering to a subject the inhibitory RNA in the composition of any one of numerals 321-323, the method comprising administering to the subject an effective amount of the composition.
332. The method of numeral 330 or numeral 331, the inhibitory RNA being against a endogenous messenger RNA encoding a endogenous protein of the subject, and the administering decreasing the expression of the endogenous protein in the subject when compared to the expression of the endogenous protein in the subject without the administering.
333. A method of delivering to a subject the heterologous nucleic acid in the plurality of AAM RNAs of any one of numerals 298-320, the method comprising administering an effective amount of the plurality of AAM RNAs.
334. A method of delivering to a subject the heterologous nucleic acid in the composition of any one of numerals 321-323, the method comprising administering to the subject an effective amount of the composition.
335. A method of manufacturing the composition of any one of numerals 321-323 comprising encapsulating the plurality of AAM RNAs in the pharmaceutically acceptable delivery vehicle or adsorbing the plurality of AAM RNAs to the pharmaceutically acceptable delivery vehicle.
336. A use of the plurality of AAM RNAs of any one of numerals 298-320 for the manufacture of a medicament for delivering the heterologous nucleic acid, the use comprising admixing the plurality of AAM RNAs with a pharmaceutically acceptable delivery vehicle.
337. A use of the plurality of AAM RNAs of any one of numerals 313-320 for the manufacture of a medicament for preventing a disease caused by a pathogen; the pathogen comprising the immunogen; the use comprising admixing the plurality of AAM RNAs with a pharmaceutically acceptable delivery vehicle.
338. A use of the plurality of AAM RNAs of any one of numerals 313-320 for the manufacture of a medicament for treating a disease caused by a pathogen; the pathogen comprising the immunogen; the use comprising admixing the plurality of AAM RNAs with a pharmaceutically acceptable delivery vehicle.
339. The use of any one of numerals 336-338, the pharmaceutically acceptable delivery vehicle comprising a lipid nanoparticle (LNP).
340. The use of numeral 339, the LNP encapsulating the plurality of AAM RNAs.
341. The use of any one of numerals 337-340, the immunogen comprising a venom, a poison, an allergen, a cancer antigen, a bacterial antigen, a viral antigen, a fungal antigen, a parasite antigen, or a fragment thereof.
342. The use of any one of numerals 337-341, the heterologous protein comprising the antibody against the immunogen.
343. The of any one of numerals 337-341, the heterologous protein comprising the immunogen.
344. A plurality of AAM RNAs for use in eliciting an immune response to an antigen in a subject; the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.
345. A plurality of AAM RNAs for use in preventing infection by a pathogen in a subject; the pathogen producing an antigen the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.
346. A plurality of AAM RNAs for use in treating infection by a pathogen in a subject; the pathogen producing an antigen; the plurality of AAM RNAs comprising N1-methylpseudouridines, uridines, a first RNA, and one or more second RNA, the first RNA encoding the antigen, and the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a mole percentage of N1-methylpseudouridines to the total of the N1-methylpseudouridines and the uridines from 15% to 75%; the one or more admixing being under conditions wherein the RNA polymerase produces the plurality of AAM RNAs from the template nucleic acid.
347. The plurality of AAM RNAs of any one of numerals 344-346, the first RNA comprising the N1-methylpseudouridines and the uridines.
348. The plurality of AAM RNAs of any one of numerals 344-347, the one or more second RNA comprising the N1-methylpseudouridines and the uridines.
349. The plurality of AAM RNAs of any one of numerals 344-348, the subject being a human.
350. The plurality of AAM RNAs of any one of numerals 344-349, the first mole percentage being to 70%.
351. The plurality of AAM RNAs of any one of numerals 344-349, the first mole percentage being to 65%.
352. The plurality of AAM RNAs of any one of numerals 344-349, the first mole percentage being to 60%.
353. The plurality of AAM RNAs of any one of numerals 344-349, the first mole percentage being to 55%.
354. The plurality of AAM RNAs of any one of numerals 344-349, the first mole percentage being to 50%.
355. The plurality of AAM RNAs of any one of numerals 344-354, the first mole percentage being from 20%.
356. The plurality of AAM RNAs of any one of numerals 344-354, the first mole percentage being from 25%.
357. The plurality of AAM RNAs of any one of numerals 344-356, the one or more proteins capable of replicating the plurality of AAM RNAs in an intracellular environment comprising an alphavirus non-structural protein-1 (nsP1), an alphavirus nsP2, an alphavirus nsP3, and an alphavirus nsP4.
358. The plurality of AAM RNAs of any one of numerals 344-357 further comprising a poly-adenosine monophosphate (poly(A)) tail.
359. The plurality of AAM RNAs of any one of numerals 344-358 further comprising a 5′ untranslated region (5′ UTR), the 5′ UTR being 5′ of the first RNA or the second RNA.
360. The plurality of AAM RNAs of any one of numerals 344-359 further comprising a 3′ untranslated region (3′ UTR), the 3′ UTR being 3′ of the first RNA or the second RNA, and optionally being 5′ of the poly(A) tail.
361. The plurality of AAM RNAs of any one of numerals 344-360 further comprising a 5′ cap.
362. The plurality of AAM RNAs of numeral 361, the 5′ cap being a cap-0, a cap-1, or a cap-2.
363. The plurality of AAM RNAs of numeral 361, the 5′ cap being a cap-1.
364. The plurality of AAM RNAs of numeral 361, the 5′ cap being a cap-0.
365. The plurality of AAM RNAs of any one of numerals 344-364, the RNA polymerase being a T7 RNA polymerase.
366. A composition comprising the plurality of AAM RNAs of any one of numerals 344-365 and a pharmaceutically acceptable delivery vehicle.
367. The composition of numeral 366, the pharmaceutically acceptable delivery vehicle comprising a lipid nanoparticle (LNP).
368. The composition of numeral 367, the LNP encapsulating the plurality of AAM RNAs.
369. A method of manufacturing the composition of any one of numerals 344-365, the method comprising encapsulating the plurality of AAM RNAs in the pharmaceutically acceptable delivery vehicle or adsorbing the plurality of AAM RNAs to the pharmaceutically acceptable delivery vehicle.
370. A plurality of AAM RNAs for use in delivering a heterologous nucleic acid to a subject, the plurality of AAM RNAs comprising N1-methylpseudorudines, uridines, a first RNA, and a second RNA; the first RNA comprising a heterologous nucleic acid; the second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acids.
371. The plurality of AAM RNAs of numeral 370, the subject being a human.
372. The plurality of AAM RNAs of numeral 370 or numeral 371, the first mole percentage being to 70%.
373. The plurality of AAM RNAs of numeral 370 or numeral 371, the first mole percentage being to 65%.
374. The plurality of AAM RNAs of numeral 370 or numeral 371, the first mole percentage being to 60%.
375. The plurality of AAM RNAs of numeral 370 or numeral 371, the first mole percentage being to 55%.
376. The plurality of AAM RNAs of numeral 370 or numeral 371, the first mole percentage being to 50%.
377. The plurality of AAM RNAs of any one of numerals 370-376, the first mole percentage being from 20%.
378. The plurality of AAM RNAs of any one of numerals 370-376, the first mole percentage being from 25%.
379. The plurality of AAM RNAs of any one of numerals 370-378, the one or more proteins capable of replicating the plurality of AAM RNAs in an intracellular environment comprising an alphavirus non-structural protein-1 (nsP1), an alphavirus nsP2, an alphavirus nsP3, and an alphavirus nsP4.
380. The plurality of AAM RNAs of any one of numerals 370-379 further comprising a poly-adenosine monophosphate (poly(A)) tail.
381. The plurality of AAM RNAs of any one of numerals 370-380 further comprising a 5′ untranslated region (5′ UTR), the 5′ UTR being 5′ of the first RNA or the one or more second RNA.
382. The plurality of AAM RNAs of any one of numerals 370-381 further comprising a 3′ untranslated region (3′ UTR), the 3′ UTR being 3′ of the first RNA or the second RNA, and optionally being 5′ of the poly(A) tail.
383. The plurality of AAM RNAs of any one of numerals 370-382, the heterologous nucleic acid encoding a heterologous protein.
384. The plurality of AAM RNAs of any one of numerals 370-383, the heterologous nucleic acid comprising an inhibitory RNA.
385. The plurality of AAM RNAs of numeral 384, the inhibitory RNA comprising an antisense RNA, a small interfering RNA, or a microRNA.
386. The plurality of AAM RNAs of any one of numerals 383-385, the heterologous protein comprising an immunogen, an antibody against the immunogen, an immunotherapeutic molecule, or an antibody against an immune signaling molecule.
387. The plurality of AAM RNAs of any one of numerals 383-385, the heterologous protein comprising an immunogen or an antibody against the immunogen.
388. The plurality of AAM RNAs of any one of numerals 370-387 further comprising a 5′ cap.
389. The plurality of AAM RNAs of numeral 388, the 5′ cap being a cap-0, a cap-1, or a cap-2.
390. The plurality of AAM RNAs of numeral 388, the 5′ cap being a cap-1.
391. The plurality of AAM RNAs of numeral 388, the 5′ cap being a cap-0.
392. A composition comprising the plurality of AAM RNAs of any one of numerals 370-391 and a pharmaceutically acceptable delivery vehicle.
393. The composition of numeral 392, the pharmaceutically acceptable delivery vehicle comprising a lipid nanoparticle (LNP).
394. The composition of numeral 393, the LNP encapsulating the plurality of AAM RNAs.

### Definitions

Unless defined otherwise, all technical and scientific terms herein have the same meaning as commonly understood by one of ordinary skill in the art. The following terminology will be used. It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“Or” supports, contemplates, and when recited in the claims, claims “one or a combination of” as in “one or a combination of A, B, or C.” To illustrate, “A, B, or C” means A alone, B alone, C alone, the combination of A and B, the combination of A and C, the combination of B and C, and the combination of A, B, and C, unless otherwise illustrated. That is, “or” supports and contemplates “and” as in “and/or” wherein “and/or” includes any combinations within the list of alternatives without being limited solely to the combination of all alternatives in a list (i.e., “A, B, or C” includes “A and B” and is not limited to “A, B, and C”).

Furthermore, the recitation of a list of alternatives, which may be conjoined by “and” and from which at least one alternative is selected, further contemplates and supports all combinations within the list of alternatives. For example, “X is selected from the group of: A, B, and C” contemplates and supports “X is selected from the group of: A, B, C, and combinations thereof,” “X is selected from at least one of the group of: A, B, and C,” and “X is selected from one or more of the group of: A, B, and C.” For further example, “X is selected from the group consisting of A, B, and C” contemplates and supports “X is selected from the group consisting of A, B, C, and combinations thereof,” “X is selected from at least one of the group consisting of A, B, and C,” or “X is selected from one or more of the group consisting of A, B, and C.”

Each of the following contemplates and supports any of the others: “comprises,” “consists of,” “consists essentially of,” “is/are/being,” “is selected from,” “is at least selected from,” “is selected from the group of,” “is selected from the group consisting of,” “is at least selected from the group consisting of,” “is from at least one of the group consisting of,” and “is from one or more of the group consisting of.” For example and in consideration of the above regarding combinations of listed elements, recitation of “X comprises an A, a B, or a C” in the specification contemplates and supports embodiments wherein “X consists of an A, a B, or a C,” “X consists of an A, a B, a C, or combinations thereof,” “X consists of one or more of an A, a B, or a C,” “X is one or more of an A, a B, or a C,” “X is an A, a B, a C, or combinations thereof,” “X is selected from an A, a B, or a C,” “X is selected from an A, a B, a C, or combinations thereof,” “X is selected from the group consisting of an A, a B, a C, and combinations thereof,” “X is selected from at least one of the group consisting of an A, a B, and a C,” or “X is selected from one or more of the group consisting of an A, a B, and a C.” When a specific component of an embodiment is listed—e.g., “X comprises A, B, or C”-then also supported and contemplated are any embodiments which specifically exclude any individual or combinations of components—e.g., “X comprises A, but not B or C” or “X comprises A but does not comprise B or C.” “About” as used herein when referring to a measurable value such as an amount, a temporal duration, a quantum of measurement, and the like, is meant to encompass variations of .+−.20% or .+−.10%, more preferably .+−.5%, even more preferably .+−.1%, and still more preferably .+−.0.1% from the specified value, as such variations are appropriate to perform the disclosed methods.

For illustrative but non-limiting purposes, “alphavirus” includes Venezuelan equine encephalitis virus (VEE; e.g., Trinidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. In some embodiments, the term alphavirus includes chimeric alphaviruses (e.g., as described by Perri et al., (2003) J. Virol. 77(19):10394-403) that contain genome sequences from more than one alphavirus.

An “RNA replicon,” or “self-amplifying messenger RNA” is an RNA molecule which can direct its own amplification once it is introduced into the intracellular environment. The RNA replicon or self-amplifying RNA encodes one or more proteins (e.g., non-structural protein 1 (nsP1, a.k.a. non-structural alphavirus protein 1), nsP2, nsP3, and nsP4, e.g., further alphavirus nsP1-4) that are capable of amplifying the RNA replicon or self-amplifying RNA in an intracellular environment. “Capable” is used to refer to the ability of these proteins to co-opt intracellular proteins to thereby amplifying the RNA replicon or self-amplifying RNA once said RNA replicon or self-amplifying RNA are introduced into the cell, and the cell's transcriptional and translational machinery causes expression of said proteins. “Capable” is also used to refer to the requirement that the cell provide the nucleotides necessary to produce the new strand of self-amplifying RNA or RNA replicon. These one or more proteins that amplify the RNA replicon or self-amplifying RNA are encoded together or separately on one or more segments of the self-amplifying RNA or RNA replicon. These one or more RNA segments that together encode the one or more proteins that amplify the RNA are, as RNA segments, cis-acting RNA segments. The one or more proteins that amplify the RNA replicon or self-amplifying RNA are cis-acting proteins.

A “plurality of auto-amplifying messenger RNA” is a collection of RNA molecules which together can direct their own amplification once the plurality is introduced into the intracellular environment. The “plurality of auto-amplifying messenger RNA” recognizes that that which is encoded in individual segments of the self-amplifying messenger RNA or the RNA replicon can be divided out into discrete molecules and introduced together into the cell, and together these RNA encode one or more proteins capable of amplifying plurality in an intracellular environment.

In some embodiments provided is “self-amplifying messenger RNA” (SAM RNA) and in other embodiments, provided are “a plurality of auto-amplifying messenger RNA” (AAM) are claimed. In this regard, contemplated are SAM RNA or an RNA replicon, which is a stand-alone RNA molecule, (i.e., SAM RNA). Also contemplated are a plurality of auto-amplifying messenger (AAM) RNA molecules. Both the SAM RNA and the plurality of AAM RNA are contemplated to deliver a heterologous nucleic acid to a cell (i.e., a first RNA or a first RNA segment) and also deliver one or more nucleic acids that encode one or more proteins capable of replicating or amplifying the SAM RNA or the plurality of the AAM RNA in the intracellular environment. By delivering the one or more nucleic acids that encode one or more proteins capable of replicating or amplifying the SAM RNA or the plurality of the AAM RNA, it is contemplated that the one or more nucleic acids that encode one or more proteins capable of replicating or amplifying the SAM RNA or the plurality of the AAM RNA will be amplified in the cell by said proteins and that the heterologous nucleic acid will be amplified in the cell by said proteins. In this regard, the SAM RNA is self-amplifying. The plurality of the AAM RNA, as a collective of RNA, is also self-amplifying. However, in some embodiments of the plurality of AAM RNA, the heterologous nucleic acid is separated on a stand-alone nucleic acid molecule from the one or more nucleic acids that encode the one or more proteins capable of amplifying the plurality of the AAM RNA in the intracellular environment. In this embodiment, the heterologous nucleic acid is understood not to be self-amplifying by itself even though the overall composition of the plurality of the AAM RNA is self-amplifying. Therefore, “a plurality of an auto-amplifying messenger RNA” is used to refer to a plurality of RNA molecules that together have all the components (i.e., segments, regions, etc.) of the “stand-alone” self-amplifying messenger RNA (i.e., a first RNA, or a first RNA segment, and one or more second RNA, or one or more second RNA segments, wherein the first RNA, or first RNA segment, comprise a heterologous nucleic acid, and one or more second RNA, or the one second RNA segments, encode one or more proteins capable of replicating the SAM RNA or the plurality of the AAM RNA in an intracellular environment), while also, avoiding any misinterpretation wherein it is erroneously believed that each and every RNA in the plurality of the AAM RNA must be self-amplifying.

“One or more proteins capable of replicating” the RNA replicon, self-amplifying messenger RNA, or the plurality of auto-amplifying messenger RNA “in an intracellular environment” refers to those above-noted proteins that are at least as a collective capable of causing such self-amplification or auto-amplification. The example of alphavirus non-structural protein-1 (nsP1), nsP2, nsP3, and nsP4 is illustrative of such a collective. Non-structural protein 1 is an mRNA capping enzyme, which possesses both guanine-7-methyltransferase (MTase) and guanylyltransferase (GTase) activities, where they direct the methylation and capping of newly synthesized viral genomic and subgenomic RNA. These enzymes synthesize a 5′ cap on newly synthesized strands of the SAM RNA or the plurality of auto-amplifying messenger (AAM) RNA. This 5′ cap prevents the mRNA from being degraded by cellular 5′ exonucleases, thereby promoting retainment of newly synthesized strands. The 5′ cap may be essential for transcription and/or translation of any heterologous nucleic acids. But by itself, nsP1 does not synthesize new strands of nucleic acid. Non-structural protein 2 comprises a helicase for synthesis of new strands. Non-structural protein 2 provides for RNA triphosphatase activity for 5′ capping. Non-structural protein 2 also comprises a papain-like cysteine protease, which can process the preprotein comprising nsP1-4. However, by itself, nsP2 cannot replicate new strands of nucleic acid. Non-structural protein 3 does not by itself replicate new strands of nucleic acid, even though it may be important for regulating transcription from the messenger RNA. Non-structural protein 4 is a highly conserved RNA-dependent RNA polymerase. But it must be isolated from the other proteins in the preprotein comprising nsP1-4 (i.e., by the papain-like domain of nsP2). Its enzymatic activity in part might require some the helicase activity or protease activity from nsP2 or capping activity of nsP1. Accordingly, nsP1-4, collectively are capable of replicating the SAM RNA or the plurality of auto-amplifying messenger RNA in an intracellular environment.

In some embodiments, at least one of the one or more proteins that amplify or replicate the RNA replicon, self-amplifying messenger RNA, or the plurality of auto-amplifying messenger RNA in an intracellular environment, or the one or more proteins that are capable of amplifying or replicating the RNA replicon, self-amplifying messenger RNA, or the plurality of auto-amplifying messenger RNA once the RNA replicon, self-amplifying messenger RNA, or the plurality of auto-amplifying messenger RNA are/is in an intracellular environment, are from an alphavirus. In some embodiments, the one of the one or more proteins that amplify the RNA replicon, self-amplifying RNA, or the plurality of auto-amplifying messenger RNA are from an alphavirus. In other embodiments, at least one of the one or more proteins that amplify the RNA replicon, self-amplifying messenger RNA, or the plurality of auto-amplifying messenger RNA are from a virus other than an alphavirus. In other embodiments, the one or more proteins that amplify the RNA replicon, self-amplifying messenger RNA, or the plurality of auto-amplifying messenger RNA are from a virus other than an alphavirus. In some embodiments, the virus other than an alphavirus comprises a positive-stranded RNA viruses. In some embodiments, the virus other than an alphavirus that is a positive-strand RNA virus comprises as a picornavirus, flavivirus, rubivirus, pestivirus, hepacivirus, calicivirus, or coronavirus.

In some embodiments, the one or more proteins capable of replicating the RNA replicon, self-amplifying messenger RNA, or the plurality of auto-amplifying messenger RNA in an intracellular environment comprise virally-derived cis-acting elements which provide for said self-amplification, self-replication, or auto-amplification in an intracellular environment.

Suitable wild-type cis-acting alphavirus sequences are well-known and are available from sequence depositories, such as the American Type Culture Collection, 10801 University Boulevard, Manassas, Virginia, U.S. Representative examples of suitable alphaviruses include (by ATCC deposit number) Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro virus (ATCC VR-66; ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

The term “conservative sequence modifications” within the context of amino acid sequences refers to amino acid modifications that do not significantly affect or alter the binding characteristics of the antigen, immunogen, protein, antibody, or antibody fragment containing the amino acid sequence. Such conservative modifications include amino acid substitutions, additions, and deletions. Modifications can be introduced into an antigen, an immunogen, a protein, an antibody, or an antibody fragment by, for example, site-directed mutagenesis and PCR-mediated mutagenesis. Conservative amino acid substitutions are ones in which the amino acid residue is replaced with an amino acid residue having a similar side chain. Similar side chains include categorization by substitution of and with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine), and aromatic side chains (e.g., tyrosine, phenylalanine, tryptophan, histidine).

“Sequence,” “segment,” “nucleic acid,” or “region” as used within the context of a nucleic acid includes sense (i.e., positive) and anti-sense (i.e., negative, e.g., reverse complementary) sequences of the same nucleic acid. A “segment,” “sequence,” “nucleic acid,” or “region” that “encodes” a coding sequence, wherein the coding sequence is transcribed and/or translated, includes sense and antisense (e.g., reverse complementary) sequences of the same nucleic acid. In some embodiments, the coding sequences encode a heterologous protein. In some embodiments, the heterologous protein comprises an immunogen (a.k.a. antigens). In some embodiments, the coding sequence encodes an antibody. In some embodiments, the antibody is an antibody against the immunogen or antigen. In some embodiments, the immunogen or antigen comprises a venom, a poison, an allergen, a cancer antigen, a bacterial antigen, a viral antigen, a fungal antigen, a parasite antigen, or a fragment thereof. That is, in some embodiments, the antibody against an immunogen or an antigen is an antibody against a venom, a poison, an allergen, a cancer antigen, a bacterial antigen, a viral antigen, a fungal antigen, a parasite antigen, or a fragment thereof. And in some embodiments, the coding sequence encodes an immunogen or an antigen, which is a venom, a poison, an allergen, a cancer antigen, a bacterial antigen, a viral antigen, a fungal antigen, a parasite antigen, or a fragment thereof.

In some embodiments, the coding sequences encode heterologous proteins. In some embodiments, the heterologous protein can comprise an immunotherapeutic molecule or an enzyme. In some embodiments, the enzyme comprises a galactose-1-phosphate uridylyltransferase (GALT) or acid sphingomyelinase, which would be administered to a subject having reduced or deficient activity for the native enzyme, i.e., someone having galactosemia or Neiman-Pick disease respectively. In some embodiments, the heterologous protein is a coagulation factor. In some embodiments, the coagulation factor is protein C thrombomodulin, Protein S, active protein C, Factor I, Factor IA, Factor Prothombin, Factor Thrombin, Antithrombin, Tissue Factor, Factor VII, Factor VIIa, Factor X, Factor Xa, Factor XI, Factor XIa, Factor XII, Factor XIIa, Factor XIII, Factor XIIIa, Factor IX, Factor IXa, Factor VIII, Factor VIIIa, Factor XII, Factor XIIa, Factor FV, or Factor FVa.

In some embodiments, a “segment,” “sequence,” “nucleic acid,” or “region” that encodes a coding sequence, is a segment, sequence, or region that encodes an immunogen (a.k.a. antigen) or encodes a protein. In some embodiments, the protein includes an antibody. Likewise, a “segment,” “sequence,” “nucleic acid,” or “region” that “encodes” a non-coding sequence, such as miRNA or a promoter, also includes sense and antisense (e.g., reverse complementary) sequences of the same nucleic acid. This inclusion of both sense and antisense strands into the segment, sequence, nucleic acid, or region is due to the property of a nucleic acid to undergo semi-conservative replication whereby genetic information is retained. In semi-conservative replication, the two strands of double-stranded nucleic acid are separated (i.e., melt or are separated by a helicase), and each of the two strands are used as a template from which a newly synthesized, reverse complementary strand is formed. That is, in semi-conservative replication, the genetic information, whether as sense or antisense is preserved, and a protein, immunogen, miRNA, or promoter, for example, may be produced from the initial strand or strands synthesized therefrom regardless of whether the initial strand is sense or antisense. Within the context of self-amplifying RNA, semi-conservative replication provides for the propagation of, for example, the protein, immunogen, miRNA, or promoter regardless of whether the segment encoding the protein, immunogen, miRNA, or promoter was sense or antisense. In this regard, it is understood that the SAM RNA can comprise one or more segments or sequences that encode one or more proteins necessary for replicating the SAM RNA in an intracellular environment, and that these segments or sequences that encode the one or more proteins necessary for replicating the SAM RNA in an intracellular environment are encoded in a sense or positive strand orientation. It is further understood that the SAM RNA can further comprise a first RNA, wherein the first RNA comprises a heterologous nucleic acid, which in some embodiments can encode a heterologous protein, and wherein the heterologous nucleic acid which in some overlapping embodiments comprises an inhibitory RNA, and it is further understood that the heterologous nucleic acid may be transcribed and translated from the antisense or negative strand of the SAM RNA.

To illustrate how “sequence,” “segment,” “nucleic acid,” or “region” as used within the context of a nucleic acid includes sense (i.e., positive) and anti-sense, if a specific sequence, called “A”, is listed as having the sequence of 5′-ATGG-3′ in the sense strand (i.e., positive strand) then it is contemplated, supported, and when listed in the claims, claimed that A also has the sequence of 3′-TACC-5′ in the antisense strand (i.e., negative strand) or complementary strand (i.e., A comprises 5′-ATGG-3′ or 3′-TACC-5′).

“Sequence,” “region,” or “segment” as used herein, unless otherwise specified, also contemplates and supports sequences incorporating different forms of nucleic acids, i.e., RNA and DNA, of the same information, or sequences incorporating differing nucleotides found in the different forms of the nucleic acids (i.e., uridines in RNA and thymidines in DNA), as well as sense and anti-sense (e.g., reverse complementary) information therein.

For example, self-amplifying RNA may be produced from plasmids of DNA, and thereby the sequence of the plasmid contemplates and supports the sequence of the self-amplifying RNA and vice versa. Since the substitution of uridines with N1-methylpseudouridines is contemplated herein, a sequence, region, or segment herein also contemplates and supports sequences incorporating analogs of uridines herein. To illustrate, if A in RNA (sense) is 5′-AUGG-3′, A also comprises 5′-ATGG-3′, being the sense DNA, and 3′-TACC-5′ being the anti-sense DNA, as well as 3′-UACC-5′, being the antisense RNA. To also illustrate, 5′-AUGG-3′ also supports and contemplates the sequence of 5′-A(N1Ψ)GG-3′, as well as 3′-(N1Ψ)ACC-5.′ To distinguish between sense and anti-sense (e.g., complementary) sequences, a prime symbol (′) may be used, i.e., for ease of tracking original genomic material, transcripts, first strand synthesis, second strand synthesis, sense, and antisense strands. To further illustrate, if a first single-stranded region comprises SEQ ID NO: 4, 5′-AATGATACGGCGACCACCGA-3′, then that first single-stranded region also supports and comprises 5′-TCGGTGGTCGCCGTATCATT-3′ (SEQ ID NO: 8).

In some embodiments, a “first RNA segment” and a “second RNA segment” are provided. It is to be understood that the second RNA segment is not necessarily downstream (3′) of the first RNA segment or that it is not necessarily upstream (5′) of the first RNA segment either. Rather, “first” or “second” with regard to an “RNA segment” is not meant to connote the order along a stand-alone molecule but rather “first” and “second” are used for nominative convenience. In this regard, a “first” or “second” or any numbered thing is to be understood to use such numbering as to differentiate between said things. For example, a first and second mole percentage may be used to distinguish between the mole percentage of something in a molecule (i.e., the mole percentage of N1-methylpseudouridines to the total of the N1-methylpseudouridines and the uridines in a SAM RNA molecule) and the mole percentage of something in an admixture (i.e., the mole percentage of N1-methylpseudouridines to the total of the N1-methylpseudouridines and the uridines in an in vitro transcription reaction).

“Nucleic acid,” “polynucleotide,” and “oligonucleotide” as used herein all have the same meaning and they are inherently composed of a sequence of nucleotides, each nucleotide comprising a phosphate and a nucleoside, a nucleoside comprising a pentose sugar (e.g., deoxyribose and ribose) and a nucleobase (e.g., a purine comprising adenine or guanine and a pyrimidine comprising cytosine, uracil, N1-methyluracil, and thymine). In one aspect, the sugar and nucleobase can be covalently bound as cytidine, thymidine, guanosine, adenosine, uridine, pseudouridine (a.k.a. 5-(β-D-Ribofuranosyl)pyrimidine-2,4(1H,3H)-dione or 5-[(2S,3R,4S,5R)-3,4-Dihydroxy-5-(hydroxymethyl)oxolan-2-yl]pyrimidine-2,4(1H,3H)-dione, CAS No. 1445-07-4, PubChem CID 15047), N1-methyluridine, N1-methylpseudouridine (a.k.a. 5-[(2S,3R,4S,5R)-3,4-Dihydroxy-5-(hydroxymethyl)oxolan-2-yl]-1-methylpyrimidine-2,4-dione, CAS No. 13860-38-3, PubChem CID 99543), or deoxyribose-containing or ribose-containing forms thereof. A “nucleic acid,” “polynucleotide,” and “oligonucleotide” can be a stand-alone molecule (i.e., an RNA molecule) or they may be “region,” “sequence,” or “segment” therein, and in this regard, the use of “region,” “sequence,” or “segment” is used to distinguish between such and a stand-alone molecule.

The term “immunogen” or also known as an “antigen” (Ag) refers to a molecule that provokes an immune response and can be bound to a protein comprising complementary-determining regions such as an antibody or a T-cell receptor. An antibody includes a B-cell receptor (i.e., an antibody complexed with CD79A and CD79B). This immune response may involve either antibody production against the antigen (i.e., antibody-antigen binding), or the activation of specific immunologically-competent cells to the antigen (i.e., T-cell receptor binding to the antigen), or both. Any macromolecule, including virtually all proteins or peptides, and further including all proteins and peptides comprising post-translational modifications such as the additions of sugars, lipids, and combinations thereof, can serve as an antigen. Antigens can be derived from recombinant or genomic nucleic acids. Any nucleic acid, which comprises a nucleotide sequences or a partial nucleotide sequence encoding a protein that elicits an immune response thereby encodes an “antigen” or “immunogen.” In some embodiments, an antigen or immunogen is encoded by a full-length nucleotide sequence of a gene. In some embodiments, antigens or immunogens are encoded by a partial nucleotide sequence and partial nucleotide sequences of more than one gene. In this regard, in some embodiments, the antigen or immunogen is the full-length native protein or proteins, and in some embodiments, the antigen or immunogen is a truncated portion of the full-length native protein or proteins. In some embodiments, these nucleotide sequences are arranged in various combinations to encode polypeptides that elicit the desired immune response. In this regard, an antigen need not be encoded by a “gene” at all. An antigen can be generated, synthesized, or derived from a biological sample and the amino acid sequence of the protein antigen might be reverse translated or codon optimized to generate a polynucleotide sequence that then encodes the antigen. With regard to the antigen being a full-length or truncated protein and with regard to the antigen activating a T-cell, it is understood that the cells expressing the SAM RNA or the plurality of the AAM RNA will express process the antigen in the intracellular environment by truncating the antigen into lengths that can be expressed on by a major histocompatibility receptor so that the T-cell receptor may recognize the MHC-presented antigen.

Such a biological sample can include, but is not limited to a pathogen, a tissue sample, a tumor sample, a cell, or a fluid with other biological components. The pathogen can include a bacteria or a virus. In some embodiments, the immunogen or antigen comprises a venom, a poison, an allergen, a cancer antigen, a bacterial antigen, a viral antigen, a fungal antigen, a parasite antigen, or a fragment thereof.

The term “antibody,” as used herein, refers to an immunoglobulin molecule, which comprises three heavy-chain complementary-determining regions and three light-chain complementary-determining regions (collectively an antigen-determining region), and therefrom specifically binds with an antigen. An antibody can comprise the quintessential “Y” shaped immunoglobulin which comprises two arms and a stem and which comprises two heavy-chains and two light-chains. Each arm comprises a variable region which comprises said light-chain and heavy-chain complementary-determining regions, and each arm comprising a light-chain and a portion (C_{H}1 region) of the heavy-chain. Each stem comprises two portions of a heavy-chain, each portion comprising a C_{H}2 region and a C_{H}3 region. That is, antibodies can be intact immunoglobulins derived from natural sources or from recombinant sources and can be immunoreactive portions of intact immunoglobulins. Antibodies can also be fragments of said intact antibody, wherein the fragment comprises said three heavy-chain complementary-determining regions and said three light-chain complementary-determining regions (i.e., said antigen-determining regions), and therefrom specifically binds with an antigen. The antibodies may exist in a variety of forms including, for example, Fv, Fab, F(ab)₂, linear antibodies, and single chain antibodies (scFv). Antibodies can include polyclonal antibodies, monoclonal antibodies, humanized antibodies, human antibodies, bispecific antibodies, and multi-specific antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The terms “protein”, “polypeptide” and “peptide” are used interchangeably herein and refer to any peptide-linked chain of amino acids, regardless of length or post-translational modification (e.g., phosphorylation or addition of sugars, lipids, or combinations thereof).

The term “post-translational” used herein refers to events that occur after the translation of a nucleotide triplet into an amino acid and the formation of a peptide bond to the proceeding amino acid in the sequence. Such post-translational events may occur after the entire polypeptide was formed or already during the translation process on those parts of the polypeptide that have already been translated. Post-translational events typically alter or modify the chemical or structural properties of the resultant polypeptide. Examples of post-translational events include the addition of sugars, lipids, phospho-groups, cleavage of the peptide chain, or restructuring of folding by, for example, heat shock proteins.

The term “co-translational” used herein refers to events that occur during the translation process of a nucleotide triplet into an amino acid chain. Those events typically alter or modify the chemical or structural properties of the resultant amino acid chain. Examples of co-translational events include but are not limited to events that may stop the translation process entirely or interrupted the peptide bond formation resulting in two discreet translation products.

As used herein, the terms “polyprotein” or “artificial polyprotein” refer to an amino acid chain that comprises, or essentially consists of or consists of two amino acid chains that are not naturally connected to each other. The polyprotein may comprise one or more further amino acid chains. Each amino acid chain is preferably a complete protein, i.e., spanning an entire ORF, or a fragment, domain or epitope thereof. The individual parts of a polyprotein may either be permanently or temporarily connected to each other. Parts of a polyprotein that are permanently connected are translated from a single ORF and are not later separated co- or post-translationally. Parts of polyproteins that are connected temporarily may also derive from a single ORF but are divided co-translationally due to separation during the translation process or post-translationally due to cleavage of the peptide chain, e.g., by an endopeptidase. Additionally or alternatively, parts of a polyprotein may also be derived from two different ORF and are connected post-translationally, for instance through covalent bonds.

An “epitope”, also known as antigenic determinant, is the segment of a macromolecule that is recognized by the immune system, specifically by antibodies or TCRs, (e.g., by B cells, or T cells). Such epitope is that part or segment of a macromolecule capable of binding to an antibody or antigen-binding fragment thereof. In this context, the term “binding” preferably relates to a specific binding. In the context of the present invention it is preferred that the term “epitope” refers to the segment of protein or polyprotein that is recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

“Protect” or “protection” in the context of protecting against infection, diseases, or conditions caused by a pathogen in a subject means to produce either directly (i.e., by encoding an antibody) or indirectly (i.e., by encoding an antigen to which the immune system responds by producing anti-antigen antibodies or anti-antigen TCR-mediated immune responses) or to elicit an immune response that decreases the likelihood of: 1) the host's body being a reservoir for the replication of the pathogen, and/or to such a level of replication that it can pass from that host to another, or 2) the likelihood or severity of a symptom or a reduction in the number of symptoms of infection by said pathogen. In some embodiments, protection reduces the incidence of an infection, disease, or condition caused by pathogen (i.e., whether symptomatic and asymptomatic) possibly leading to the control of the disease associated with said-pathogen (i.e., sudden acute respiratory syndrome coronavirus-2 (SARS-CoV-2) causing the disease known as coronavirus virus disease 2019 (COVID-19) and/or to the control of associated adverse health outcomes caused by the pathogen.

“Treat” or “treatment” in the context of infection, diseases, or conditions caused by a pathogen means to treat via administration, post-infection any pathogen-causing symptom, effect, or phenotype. Treatment may mean to decrease the severity or frequency of symptoms of the condition or disease in a subject, slow or eliminate the progression of the condition, totally or partially eliminate the symptoms of the disease or condition in the subject, or reduce or eliminate the number of pathogens in the subject. Treatment of an infection, disease, or condition caused by a pathogen includes ameliorating, stabilizing, reducing, or eliminating the symptoms, effects, or phenotypes caused by the pathogen.

In the different aspects, there is provided a self-amplifying messenger (SAM) ribonucleic acid (RNA) and a plurality of auto-amplifying messenger (AAM) RNA. In each aspect, each of these RNAs have a mole percentage of N1-methylpseudouridines to the total of N1-methylpseudouridines and uridines. In other aspects, each of these RNAs were produced from admixtures having a mole percentage of N1-methylpseudouridines to the total of N1-methylpseudouridines and uridines. In embodiments therein, each of these RNA may have heterologous nucleic acids and RNA segments or RNAs that encode one or more proteins capable of replicating the RNA in an intracellular environment. In embodiments therein, each of these RNA may have additional structures such as subgenomic promoters, poly adenosine monophosphate tails, 5′ caps, 5′ upstream terminal repeats, 3′ terminal repeats, and further embodiments further specifying each of these components, such as a cap-1, the heterologous nucleic acid encoding a heterologous protein, the heterologous protein comprising an antigen, etc. In several aspects, the SAM RNA or the plurality of AAM RNA may have uses in the manufacture of medicaments, such as those for delivering a heterologous nucleic acid, for preventing a disease, for treating a disease, for delivering a heterologous protein, or for delivering an inhibitory RNA. Or in several aspects, the SAM RNA or the plurality of AAM RNA may have uses in eliciting an immune response, preventing infection, treating infection, or delivering a heterologous protein, a heterologous nucleic acid, or an inhibitory RNA. Or in several aspects, the SAM RNA or the plurality of AAM RNA may be comprised within a composition, may be manufactured, may be administered in a method having the above-noted uses. Throughout all of the above-noted aspects and embodiments therein, it is to be understood that disclosure of embodiments that support one aspect are intended to, and do, support embodiments of the other aspects. This statement is not limited to supporting across the SAM RNA and the plurality of AAM RNA, but may be used to support and contemplate between compositions of matter, uses of matter, manufacture of the matter, methods of administering the matter, etc. For example, a 5′ cap to the SAM RNA may be used to support embodiments to the 5′ cap of the plurality of AAM RNA, or for example, a method of producing a 5′ capped RNA may be used to support embodiments to the 5′ cap RNA, or methods of making the SAM RNA or AAM RNA may be used to support the use of the SAM RNA or the AAM RNA for the manufacture of a medicament.

### Self-Amplifying RNAs

In one aspect, a self-amplifying messenger (SAM) ribonucleic acid (RNA) is provided. In some embodiments, the SAM RNA comprises N1-methylpseudourdines, uridines, a first RNA, and a second RNA. In some embodiments, the first RNA comprises a heterologous nucleic acid. In some embodiments, the heterologous nucleic acid encodes a heterologous protein. In some embodiments, heterologous nucleic acid comprises an inhibitory RNA. In some embodiments, heterologous nucleic acid comprises an inhibitory RNA and encodes a heterologous protein. In some embodiments, the inhibitory RNA comprises an antisense RNA, a small interfering RNA, or a microRNA. In some embodiments, the heterologous protein comprises an immunogen, an antibody, or an immunotherapeutic molecule. In some embodiments, the heterologous protein comprises an immunogen or an antibody against the immunogen encodes an immunogen or an antigen. In some embodiments, the heterologous protein comprises an antigen or an antibody against the antigen.

In some embodiments, the SAM RNA comprises SEQ ID NO.: 1.

In some embodiments, the first RNA or first RNA segment comprises two or more heterologous nucleic acids. In some embodiments, the heterologous protein is extended pharmacokinetic (PK) interleukin (IL)-2 or extended pharmacokinetic (PK) interleukin (IL)-7. In some embodiments, the heterologous protein is a peptide or protein comprising an epitope for inducing an immune response against an antigen in the subject. In some embodiments, first RNA or first RNA segment encodes a heterologous protein, which is a peptide or protein comprising an epitope for inducing an immune response against an antigen in the subject, and a heterologous protein, which is extended pharmacokinetic (PK) interleukin (IL)-2 or extended pharmacokinetic (PK) interleukin (IL)-7. In some embodiments, the heterologous protein is a first fusion protein comprising extended PK-IL-2 or extended-PK IL-7. In some embodiments, the first fusion protein comprises an IL2 moiety and a moiety selected from: serum albumin, an immunoglobulin fragment, transferrin, Fn3, variants thereof, and combinations thereof.

In some embodiments, the heterologous protein comprises interleukin-12sc (IL-12sc), IL-15sushi, IFNα, or GM-CSF. In some embodiments, the heterologous protein comprise octamer-binding transcription factor-3/4 (OCT3/4), (sex determining region Y)-box-2 (SOX-2), Kruppel-like factor-4 (KLF-4), cellular myelocytomatosis oncogene (c-MYC), LIN-28, or NANOG. In some embodiments, the heterologous protein further comprises a differentiation factor, which differentiates a pluripotent cell into at least one of a myocyte, a neurocyte, a pancreatic cell, a hepatocyte, a spleen cell, a bone marrow cell, or a skin cell.

In some embodiments, the heterologous protein comprises an antibody. In some embodiments, the antibody is tocilizumab or etanercept. In some embodiments, the antibody is an anti-IL6, anti-IL6R, anti-TNF-α, or anti-TNF receptor.

In some embodiments, the heterologous protein comprises an immunotherapeutic molecule or an enzyme. In some embodiments, the enzyme comprises a galactose-1-phosphate uridylyltransferase (GALT) or acid sphingomyelinase, which would be administered to a subject having reduced or deficient activity for the native enzyme, i.e., someone having galactosemia or Neiman-Pick disease respectively. In some embodiments, the heterologous protein is a coagulation factor. In some embodiments, the coagulation factor is protein C thrombomodulin, Protein S, active protein C, Factor I, Factor IA, Factor Prothombin, Factor Thrombin, Antithrombin, Tissue Factor, Factor VII, Factor VIIa, Factor X, Factor Xa, Factor XI, Factor XIa, Factor XII, Factor XIIa, Factor XIII, Factor XIIIa, Factor IX, Factor IXa, Factor VIII, Factor VIIIa, Factor XII, Factor XIIa, Factor FV, or Factor FVa. In some embodiments, the second RNA encodes one or more proteins capable of replicating the SAM RNA in an intracellular environment. In some embodiments, the one or more proteins capable of replicating the SAM RNA in an intracellular environment comprises an alpha virus non-structural protein-1 (nsP1), an alphavirus nsP2, an alphavirus nsP3, or an alphavirus nsP4. In some embodiments, the one or more proteins capable of replicating the SAM RNA in an intracellular environment comprises an alphavirus non-structural protein-1 (nsP1), an alphavirus nsP2, an alphavirus nsP3, and an alphavirus nsP4.

In another aspect, a SAM RNA comprising N1-methylpseudourdines, uridines, and a second RNA, but lacking nucleic acids encoding viral structural proteins, is provided. In these aspects, the SAM RNA differs from viral self-amplifying subgenomic RNA in that it may, for example, lack the capsid proteins necessary for virion packaging and cellular entry thereby.

In certain embodiments, one or more proteins capable of replicating the SAM RNA in an intracellular environment comprises an enzyme that is capable of synthesizing a 5′ cap in an intracellular environment (e.g., a 7-methylguanosine). This cap can enhance cellular retainment of the SAM RNA and can enhance transcription and translation therefrom. In some embodiments, the one or more proteins capable of replicating the SAM RNA in an intracellular environment comprises a helicase or a protease. The helicase may enhance the replicase activity (i.e., by removing secondary structures/hybridization thereby providing higher RNA-dependent RNA polymerase activity).

In some embodiments, the alphavirus comprises Venezuelan equine encephalitis virus (VEE; e.g., Trinidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, or Buggy Creek virus.

In some embodiments, the nucleic acids encoding the one or more proteins capable of amplifying the SAM RNA in an intracellular environment are from positive-strand viruses and thereby are from positive-strand nucleic acids. Accordingly, in some embodiments are provided proteins and nucleic acids encoding proteins that are positive-stranded (positive sense-stranded) RNAs which lead to translation of a replicase (or replicase-transcriptase) after delivery to a cell. In some embodiments, the replicase is translated as a polyprotein. In some embodiments, the replicase in the polyprotein auto-cleaves to provide a replication complex which creates genomic-strand copies of the positive-strand delivered RNA. Said copies would be negative sense (negative-strand) transcripts, which can be transcribed to give further copies of the positive-stranded parent RNA and also to give a subgenomic transcript which is translated to obtain the heterologous protein (i.e., an antigen, antibody, etc). Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g., the attenuated TC83 mutant of VEEV has been used in replicons, see the following reference: WO2005/113782, the content of which is incorporated by reference.

In some embodiments, the one or more proteins capable of replicating the SAM RNA in an intracellular environment comprise proteins from a positive-strand virus. In some embodiments, the positive-strand virus comprises a picornavirus, a flavivirus, a rubivirus, a pestivirus, a hepacivirus, a calicivirus, or a coronavirus.

In some embodiments, the SAM RNA is a plurality of SAM RNA, the first RNA and the second RNA being on different molecules of the SAM RNA. In some embodiments, the first RNA is a first RNA segment and the second RNA is a second RNA segment and wherein the first and the second RNA segments are on the same strand of SAM RNA.

In some embodiments, the SAM RNA further comprises a poly-adenosine monophosphate (poly(A)) tail. In some embodiments, the poly(A) tail is at the 3′ end of the SAM RNA. In some embodiments, the SAM RNA further comprises a 5′ untranslated region (5′ UTR), wherein the 5′ UTR is 5′ of the first RNA segment and the second RNA segment.

In some embodiments, the SAM RNA further comprises a 5′ untranslated region (5′ UTR), wherein the 5′ UTR is 5′ of the first RNA and the second RNA. In some embodiments, the SAM RNA further comprises a 5′ untranslated region (5′ UTR), wherein the 5′ UTR is 5′ of the first RNA or the second RNA. In some embodiments, the SAM RNA further comprises a 3′ untranslated region (3′ UTR), the 3′ UTR being 3′ of the first RNA and the second RNA, and optionally being 5′ of the poly(A) tail. In some embodiments, the SAM RNA further comprises a 3′ untranslated region (3′ UTR), the 3′ UTR being 3′ of the first RNA or the second RNA, and optionally being 5′ of the poly(A) tail. In some embodiments, the SAM RNA further comprises a 3′ untranslated region (3′ UTR), the 3′ UTR being 3′ of the first RNA segment and the second RNA segment, and optionally being 5′ of the poly(A) tail. In some embodiments, the second RNA segment is 5′ of the first RNA segment.

In some embodiments, the SAM RNA may have two open reading frames. The first (5′) open reading frame encodes the one or more proteins capable of amplifying the SAM RNA in an intracellular environment; the second (3′) open reading frame providing for transcription (and possibly translation) of the heterologous nucleic acid. In some embodiments the RNA may have additional (e.g., downstream) open reading frames, e.g., to encode further antigens or to encode accessory polypeptides.

### 5′ Capping

In some embodiments, the SAM RNA further comprises a 5′ cap. A 5′ cap comprises a guanosine connected to the SAM RNA via a 5′ to 5′ triphosphate linkage by mRNA guanylyltransferase, and wherein the guanine of said guanosine is methylated at its 7 position. In this context, a 5′ to 5′ triphosphate linkage occurs when the 5′ end of the ribose of said guanosine is linked to the 5′ end of the ribose of the SAM RNA via a triphophosphate group by mRNA guanylyltransferase. Thereafter, the guanine of said guanosine is methylated at its 7 position by (guanine-N7-)-methyltransferase. Without further methylation, this cap is known as cap-0 and is expressed as 5′(m7Gp)(ppN)[pN]_{N}, wherein the former “N” indicates the first (5′) nucleobase of the SAM RNA, the “pN” indicates a further nucleotide in the SAM RNA, and the addition of “[..]_{N}” in “[pN]_{N}” indicates the repeating polymeric structure of the SAM RNA and thereby collectively each sequentially adjacent nucleotide in the SAM RNA.

An additional oxygen-linked methylation by a 2′-O-methyltransferase to the 2′ carbon of the ribose of the nucleotide of the SAM RNA immediately adjacent to said guanosine results in a cap-1 structure, which is expressed as 5′(m7Gp)(ppm2N)[pN]_{N}, wherein the addition of the “m2” indicates the oxygen-linked methylation of the 2′ carbon of the ribose of the nucleotide immediately adjacent (via the triphosphate linkage) to said guanosine. And further still, an additional methylation to the next (3′) nucleotide of the SAM RNA immediately adjacent to the nucleotide methylated in cap-1 results in a cap-2 structure, which is expressed as 5′(m7Gp)(ppm2N)(m2pN)[pN]n, wherein the addition of the latter “m2” indicates the methylation of the nucleotide immediately adjacent to the nucleotide methylated in cap-1. This cap-2 methylation is also to the 2′ carbon of the ribose of that immediately adjacent nucleotide. In some embodiments, the 5′ cap is a cap-0, a cap-1, or a cap-2. In some embodiments, the 5′ cap is a cap-0. In some embodiments, the 5′ cap is a cap-1. In some embodiments, the 5′ cap is a cap-2.

Kits providing all of the materials for a 5′ cap, whether it is cap-1 or cap-2, and supplemental kits adding cap-1 and cap-2 capacity to a cap-0 kit can be used. The methods for 5′ capping can be carried out according to the manufacturer's instructions.

### Subgenomic Promoters

In some embodiments, SAM RNA comprises one or more viral subgenomic “junction region” promoters or subgenomic promoters directing the expression of heterologous nucleotide sequences, which may, in certain embodiments, be modified in order to increase or reduce viral transcription of the subgenomic fragment and heterologous sequence(s) to be expressed. Other control elements can be used, as described below. Subgenomic promoters, also known as junction region promoters, can be used to regulate protein expression. Alphaviral subgenomic promoters regulate expression of alphaviral structural proteins. See Strauss and Strauss, “The alphaviruses: gene expression, replication, and evolution,” Microbiol Rev. 1994 September; 58(3):491-562. A polycistronic polynucleotide can comprise a subgenomic promoter from any alphavirus. When two or more subgenomic promoters are present in a polycistronic polynucleotide, the promoters can be the same or different. For example, the subgenomic promoter can have the sequence CTCTCTACGGCTAACCTGAATGGA (SEQ ID NO: 3). In certain embodiments, subgenomic promoters can be modified in order to increase or reduce viral transcription of the proteins. See U.S. Pat. No. 6,592,874.

### Order of Components

In some embodiments, the SAM RNA comprises any of the following: the 5′ cap, the 5′ UTR, a first subgenomic promoter, the second RNA segment, a second subgenomic promoter, the first RNA segment, the 3′ UTR, and a poly(A) tail. In some embodiments, the SAM RNA comprises all of the following: the 5′ cap, the 5′ UTR, a first subgenomic promoter, the second RNA segment, a second subgenomic promoter, the first RNA segment, the 3′ UTR, and a poly(A) tail. In some embodiments, the SAM RNA comprises all of the following and in the following order: 5′- the 5′ cap, the 5′ UTR, a first subgenomic promoter, the second RNA segment, a second subgenomic promoter, the first RNA segment, the 3′ UTR, and a poly(A) tail-3′.

### Percentage of Uridine to N1-Methylpseudouridine Substitution in the SAM RNA

In some embodiments, the SAM RNA has a mole percentage (i.e., a first mole percentage within the context of the SAM RNA) of the N1-methylpseudouridines to the total of N1-methylpseudouridines and uridines from 15% to 75%. In some embodiments, the SAM RNA has a mole proportion (i.e., a first mole proportion within the context of the SAM RNA) of the N1-methylpseudouridines to the total of N1-methylpseudouridines and uridines from 0.15% to 0.75. In some embodiments, the SAM RNA has a mole ratio (i.e., a first mole ratio within the context of the SAM RNA) of the N1-methylpseudouridines to the uridines from 15:85 to 75:25.

As noted above, the SAM RNA comprises uridines and N1-methylpseudouridines. Thereby, the SAM RNA comprises a mole proportion or a mole percentage of the N1-methylpseudouridines to the total of the uridines and the N1-methylpseudouridines. For example, the SAM RNA can inherently comprise a first mole proportion of the N1-methylpseudouridines to the total of the uridines and the N1-methylpseudouridines, or the SAM RNA can inherently comprise a first mole percentage of the N1-methylpseudouridines to the total of the uridines and the N1-methylpseudouridines. Or, the SAM RNA inherently comprises a mole ratio of the N1-methylpseudouridines to the uridines. For example, the SAM RNA can inherently comprise a first mole ratio, being the mole ratio of the N1-methylpseudouridines to the uridines.

In other overlapping embodiments, the SAM RNA is produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid. That is, whatever mole percentage of the N1-methylpseudouridines to the total of the N1-methylpseudouridines and the uridines in the SAM RNA is determined by the mole percentage of the N1-methylpseudouridines to the total of the N1-methylpseudouridines and the uridines in admixture that obtains the SAM RNA. In some embodiments, the admixture has a mole proportion (i.e., a first mole proportion within the context of the SAM RNA) of the N1-methylpseudouridines to the total of N1-methylpseudouridines and uridines from 0.15% to 0.75. In some embodiments, admixture has a mole ratio (i.e., a first mole ratio within the context of the SAM RNA) of the N1-methylpseudouridines to the uridines from 15:85 to 75:25.

In some embodiments, the first mole percentage of N1-methylpseudouridines to the total of the uridines and the N1-methylpseudouridines, whether that first mole percentage is in the SAM RNA or in the admixture used to obtain the SAM RNA, is from 15% to 75% or from about 15% to about 75%. In some embodiments therein, the first mole percentage is from: about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%. In some embodiments therein, the first mole percentage is from: 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%. In some embodiments therein, the first mole percentage is to: about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, or about 50%. In some embodiments therein, the first mole percentage is to: 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50%. It is contemplated and supported that any of the above-noted first mole percentages that are prefixed as being “from” can be combined with any of the above-noted first mole percentages that are prefixed as being “to” (i.e., from about 18% to 73% or from 25% to about 50%).

In one embodiment, the mole percentage of the N1-methylpseudouridines to the total of the N1-methylpseudouridines and the uridines (i.e., the first mole percentage) is from 15% to 70%. In an embodiment, the mole percentage of the N1-methylpseudouridines to the total of the N1-methylpseudouridines and the uridines (i.e., the first mole percentage) is from 15% to 65%. In an embodiment, the mole percentage of the N1-methylpseudouridines to the total of the N1-methylpseudouridines and the uridines (i.e., the first mole percentage) is from 15% to 60%. In an embodiment, the mole percentage of the N1-methylpseudouridines to the total of the N1-methylpseudouridines and the uridines (i.e., the first mole percentage) is from 15% to 55%. In an embodiment, the mole percentage of the N1-methylpseudouridines to the total of the N1-methylpseudouridines and the uridines (i.e., the first mole percentage) is from 15% to 50%.

In an embodiment, the percentage of the N1-methylpseudouridines to the total of N1-methylpseudouridines and uridines is from 20% to 75%. In an embodiment, the percentage of the N1-methylpseudouridines to the total of N1-methylpseudouridines and uridines is from 25% to 75%. In an embodiment, the percentage of the N1-methylpseudouridines to the total of N1-methylpseudouridines and uridines is from 20% to 70%. In an embodiment, the percentage of the N1-methylpseudouridines to the total of N1-methylpseudouridines and uridines is from 25% to 70%. In an embodiment, the percentage of the N1-methylpseudouridines to the total of N1-methylpseudouridines and uridines is from 20% to 65%. In an embodiment, the percentage of the N1-methylpseudouridines to the total of N1-methylpseudouridines and uridines is from 25% to 65%. In an embodiment, the percentage of the N1-methylpseudouridines to the total of N1-methylpseudouridines and uridines is from 20% to 60%. In an embodiment, the percentage of the N1-methylpseudouridines to the total of N1-methylpseudouridines and uridines is from 25% to 60%. In an embodiment, the percentage of the N1-methylpseudouridines to the total of N1-methylpseudouridines and uridines is from 20% to 55%. In an embodiment, the percentage of the N1-methylpseudouridines to the total of N1-methylpseudouridines and uridines is from 25% to 55%. In an embodiment, the percentage of the N1-methylpseudouridines to the total of N1-methylpseudouridines and uridines is from 20% to 50%. In an embodiment, the percentage of the N1-methylpseudouridines to the total of N1-methylpseudouridines and uridines is from 25% to 50%.

In an embodiment, the percentage of the N1-methylpseudouridines to the total of N1-methylpseudouridines and uridines is from 30% to 55%. In an embodiment, the percentage of the N1-methylpseudouridines to the total of N1-methylpseudouridines and uridines is from 35% to 55%. In an embodiment, the percentage of the N1-methylpseudouridines to the total of N1-methylpseudouridines and uridines is from 40% to 55%. In an embodiment, the percentage of the N1-methylpseudouridines to the total of N1-methylpseudouridines and uridines is from 40% to 55%.

The above-noted percentages of the N1-methylpseudouridines to the total of N1-methylpseudouridines and uridines supports and contemplates the proportions of the N1-methylpseudouridines to the total of N1-methylpseudouridines and uridines and the ratios of the N1-methylpseudouridines to the uridines provided in Table 1.

TABLE 1

| Percentage | Proportion | |
|---|---|---|
| of the N1- | of the N1- | |
| methyl- | methyl- | |
| pseudo- | pseudo- | |
| uridines | uridines | |
| to | to the | Ratio of |
| the total | total | the N1- |
| of N1- | of N1- | methyl- |
| methyl | methyl- | pseudo- |
| pseudo- | pseudo- | uridines |
| uridines- | uridines | to |
| and | and | the |
| uridines | uridines | uridines |
| 15 | 0.15 | 15:85 |
| 16 | 0.16 | 16:84 |
| 17 | 0.17 | 17:83 |
| 18 | 0.18 | 18:82 |
| 19 | 0.19 | 19:81 |
| 20 | 0.2 | 20:80 |
| 21 | 0.21 | 21:79 |
| 22 | 0.22 | 22:78 |
| 23 | 0.23 | 23:77 |
| 24 | 0.24 | 24:76 |
| 25 | 0.25 | 25:75 |
| 26 | 0.26 | 26:74 |
| 27 | 0.27 | 27:73 |
| 28 | 0.28 | 28:72 |
| 29 | 0.29 | 29:71 |
| 30 | 0.3 | 30:70 |
| 31 | 0.31 | 31:69 |
| 32 | 0.32 | 32:68 |
| 33 | 0.33 | 33:67 |
| 34 | 0.34 | 34:66 |
| 35 | 0.35 | 35:65 |
| 36 | 0.36 | 36:64 |
| 37 | 0.37 | 37:63 |
| 38 | 0.38 | 38:62 |
| 39 | 0.39 | 39:61 |
| 40 | 0.4 | 40:60 |
| 41 | 0.41 | 41:59 |
| 42 | 0.42 | 42:58 |
| 43 | 0.43 | 43:57 |
| 44 | 0.44 | 44:56 |
| 45 | 0.45 | 45:55 |
| 46 | 0.46 | 46:54 |
| 47 | 0.47 | 47:53 |
| 48 | 0.48 | 48:52 |
| 49 | 0.49 | 49:51 |
| 50 | 0.5 | 50:50 |
| 51 | 0.51 | 51:49 |
| 52 | 0.52 | 52:48 |
| 53 | 0.53 | 53:47 |
| 54 | 0.54 | 54:46 |
| 55 | 0.55 | 55:45 |
| 56 | 0.56 | 56:44 |
| 57 | 0.57 | 57:43 |
| 58 | 0.58 | 58:42 |
| 59 | 0.59 | 59:41 |
| 60 | 0.6 | 60:40 |
| 61 | 0.61 | 61:39 |
| 62 | 0.62 | 62:38 |
| 63 | 0.63 | 63:37 |
| 64 | 0.64 | 64:36 |
| 65 | 0.65 | 65:35 |
| 66 | 0.66 | 66:34 |
| 67 | 0.67 | 67:33 |
| 68 | 0.68 | 68:32 |
| 69 | 0.69 | 69:31 |
| 70 | 0.7 | 70:30 |
| 71 | 0.71 | 71:29 |
| 72 | 0.72 | 72:28 |
| 73 | 0.73 | 73:27 |
| 74 | 0.74 | 74:26 |
| 75 | 0.75 | 75:25 |

### Compositions Comprising a SAM RNA

In some embodiments, a composition is provided, the composition comprising any of the above-noted the SAM RNAs and a pharmaceutically acceptable delivery vehicle. In some embodiments, the pharmaceutically acceptable delivery vehicle can be selected for the route of administration, and would include those formulations for oral, intravenous, intraperitoneal, subcutaneous, pulmonary, transdermal, intramuscular, intranasal, buccal, sublingual, or suppository administration. In some embodiments, the cell populations are administered parenterally. The term “parenteral” includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH (i.e., acidic). In some embodiments, the pharmaceutically acceptable delivery vehicles can be selected based on the rule of five for non-oral routes (i.e., ophthalmic, inhalation, or transdermal administration).

In some embodiments, a composition is provided, the composition comprising any of the above-noted the SAM RNAs and a polyalkyleneimine. In some embodiments, the molar ratio of the number of nitrogen atoms (N) in the polyalkyleneimine to the number of phosphor atoms (P) in the single stranded RNA (N:P ratio) is 2.0 to 15.0. In some embodiments, the composition has an ionic strength of 50 mM or less.

In some embodiments, compositions include the constructs, nucleic acid sequences, and/or polypeptide sequences described elsewhere herein in plain water (e.g., “w.f.i.” or “water for injection”) or in a buffer e.g., a phosphate buffer, a Tris buffer, a borate buffer, a succinate buffer, a histidine buffer, or a citrate buffer. Buffer salts will typically be included in the 5-20 mM range. In some embodiments, compositions have a pH between 5.0 and 9.5 e.g., between 6.0 and 8.0. In some embodiments, compositions may include sodium salts (e.g., sodium chloride) to give tonicity. A concentration of 10 mg/mL+/−.2 mg/mL NaCl is typical, e.g., about 9 mg/mL. In some embodiments, compositions include metal ion chelators. These can prolong RNA stability by removing ions which can accelerate phosphodiester hydrolysis. Thus, in some embodiments, a composition comprises EDTA, EGTA, BAPTA, or pentetic acid. Such chelators are typically present at between 10-500 μM, e.g., 0.1 mM. A citrate salt, such as sodium citrate, can also act as a chelator, while advantageously also providing buffering activity. In some embodiments, compositions have an osmolality of between 200 mOsm/kg and 400 mOsm/kg, e.g., between 240-360 mOsm/kg, or between 290-310 mOsm/kg. In some embodiments, compositions comprise a preservative, such as thiomersal or 2-phenoxyethanol. In some embodiments, the preservative is mercury-free. In some embodiments, the composition is preservative-free. In some embodiments, the composition is aseptic or sterile. In some embodiments, the composition is non-pyrogenic, e.g., containing <1 EU (endotoxin unit, a standard measure) per dose, and preferably <0.1 EU per dose. In some embodiments, the compositions is gluten-free. In some embodiments, the composition is in unit dose form. In some embodiments, a unit dose may be formulated to provide an effective amount in a volume from 0.1 mL to 1.0 mL, e.g., about 0.5 mL.

In some embodiments, the compositions disclosed herein are immunogenic composition that, when administered to a subject, induce a humoral and/or cellular antigen-specific immune response (i.e., an immune response which specifically recognizes a naturally occurring antigen from a pathogen). For example, an immunogenic composition may induce a memory T and/or B cell population that responds to the antigen or immunogen and thereby the pathogen to neutralize the antigen, immunogen, or pathogen, or cause a T_{H1} or T_{H2} response. In some embodiments, the composition may be formulated as a vaccine (i.e., to elicit an immune response wherein the immune response is a protective immune response) and in other embodiments, the composition may be formulated as a therapeutic (i.e., to elicit an immune response wherein the immune response is a therapeutic immune response, i.e., to prevent re-emergence of a latent virus).

In some embodiments, the composition can be formulated as vaccine composition. The vaccine will comprise an immunologically effective amount of the first nucleic acid which encodes an antigen or an antibody against the antigen. By “an immunologically effective amount” is intended that the administration of that amount to a subject, either in a single dose or as part of a series, is effective for inducing a measurable immune response against the antigen and thereby the pathogen that produces said antigen. This amount varies depending upon the health and physical condition of the individual to be treated, age, the taxonomic group of individual to be treated (e.g., human, non-human primate, etc.), the capacity of the individual's immune system to synthesize antibodies, the degree of protection desired, the formulation of the composition or vaccine, the treating doctor's assessment of the medical situation, the severity of the disease, the potency of the compound administered, the mode of administration, and other relevant factors. The amount will fall in a relatively broad range that can be determined through trials. In one embodiment, an immunologically effective amount of a first RNA that encodes an immunogen or antigen is an amount sufficient to prevent or treat infection by the pathogen that produces the antigen or immunogen. Vaccines as disclosed herein may either be prophylactic (i.e., to induce a protective immune response to the pathogen) or therapeutic (i.e., to treat infection). In some embodiments, the vaccine compositions disclosed herein may induce an effective immune response against the pathogen, i.e., a response sufficient for treatment or sufficient for eliciting a protective immune response to infection by the pathogen.

In some embodiments, the composition further comprises an additional antigen, a nucleic acid encoding the antigen, or a nucleic acid encoding an antibody against the antigen. In some embodiments, the composition is administered to a subject in combination with a further composition which comprises an additional antigen, a nucleic acid encoding the antigen, or a nucleic acid encoding an antibody against the antigen.

In some embodiments, the composition comprises, or be administered in conjunction with, one or more adjuvants (e.g., vaccine adjuvants). By “adjuvant” is intended that is capable of increasing an immune response against an antigen compared to administration of said antigen alone or nucleic acid encoding said antigen alone. In some embodiments, compositions further comprise one or more immunostimulants, for example, a saponin such as QS21. In some embodiments, the addition of cholesterol to the composition (i.e., to the LNP) can be used to reduce any hemolytic activity, or toxicity, from the saponin.

Adjuvants include, but are not limited to: (A) mineral-containing compositions, for example aluminum and calcium salts, such as aluminum phosphates; (B) oil emulsions, for example squalene-in-water emulsions, such as MF59, AS03, complete Freund's adjuvant (CFA), and incomplete Freund's adjuvant (IF A); (C) saponin formulations; (D) virosomes and virus-like particles (VLPs); (E) bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof; (F) human immunomodulators, for example cytokines, such as interleukins, interferons, macrophage colony stimulating factor, and tumor necrosis factor; (G) bioadhesives and mucoadhesives, such as esterified hyaluronic acid microspheres, cross-linked derivatives of poly(acrylic acid), polyvinyl alcohol, polyvinyl pyrollidone, polysaccharides and carboxymethylcellulose; (H) microparticles, for example particles of about 100 nm to about 150 μm in diameter, more preferably about 200 nm to about 30 μm in diameter, and most preferably about 500 nm to about 10 μm in diameter) formed from materials that are biodegradable and non-toxic (e.g., a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g., with SDS) or a positively-charged surface (e.g., with a cationic detergent, such as CTAB); (I) liposomes and/or lipid nanoparticles (LNP); (J) polyoxyethylene ether and polyoxyethylene ester formulations; (K) polyphosphazene (PCPP); (L) muramyl peptides; or (M) imidazoquinolone compounds, for example imiquimod and its homologues. In this regard, the adjuvant may have additional qualities such as acting as a pharmaceutically acceptable delivery vehicle and for allowing entry of an RNA into a cell.

In some embodiments, the pharmaceutically acceptable delivery vehicle comprises a liposome or lipid nanoparticle (LNP). In some embodiments, the liposome or LNP encapsulates the SAM RNA. In some embodiments, the LNP can comprise multilamellar vesicles (MLV); small uniflagellar vesicles (SUV); or large unilamellar vesicles (LUV). MLVs have multiple bilayers in each vesicle, forming several separate aqueous compartments. SUVs and LUVs have a single bilayer encapsulating an aqueous core. SUVs typically have a diameter less than or equal to 50 nm, and LUVs have a diameter greater than 50 nm. Liposomal particles of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index <0.2. The liposome/RNA complexes of reference 1 are expected to have a diameter in the range of 600-800 nm and to have a high polydispersity. In some embodiments, the LNP comprises cholesterol. In some embodiments, the LNPs have a solid core (i.e., lack an aqueous core). In some embodiments, the LNPs comprise an aqueous core.

Various amphiphilic lipids can form bilayers in an aqueous environment to encapsulate a RNA-containing as a LNP. These lipids can be anionic, cationic, or zwitterionic. These lipids can have an anionic, cationic, or zwitterionic hydrophilic head group. Some lipids are anionic whereas other are zwitterionic and others are cationic. Suitable classes of phospholipid include, but are not limited to, phosphatidylethanolamines, phosphatidylcholines, phosphatidylserines, and phosphatidyl-glycerols. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), and 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are 1,2-dipalmitoyl-sn-glycero-3-phosphocholine (DPPC), 1,2-dioleoyl-sn-glycero-3-phosphocholine (DOPC), and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

Liposomal nanoparticles of the invention can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise 1,2-distearoyl-sn-glycero-3-phosphocholine (DSPC) (zwitterionic, saturated), DlinDMA (cationic, unsaturated), and/or 1,2-dimyristoyl-rac-glycerol (DMG) (anionic, saturated). Where a mixture of lipids is used, not all of the component lipids in the mixture need to be amphiphilic e.g., one or more amphiphilic lipids can be mixed with cholesterol.

The hydrophilic portion of a lipid can be PEGylated (i.e., modified by covalent attachment of a polyethylene glycol). This modification can increase stability and prevent non-specific adsorption of the liposomes. Various lengths of PEG can be used e.g., between 0.5-8 kDa. One example of the combination of a PEG and the above-noted lipids is 1,2-dimyristoyl-rac-glycero-3-methoxypolyethylene glycol-2000 (DMG-PEG 2000). Others include 1,2-distearoyl-sn-glycero-3-phosphoethanolamine-N-[methoxy(polyethylene glycol)-2000] (ammonium salt) and distearoyl-rac-glycerol-PEG2K. In some embodiments, the LNP comprises DSPC, DlinDMA, PEG-DMG, and cholesterol. In some embodiments, LNP comprises 10% DSPC (zwitterionic), 40% DlinDMA (cationic), 48% cholesterol and 2% PEG-conjugated DMG (2 kDa PEG) by mole.

Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

As noted above, liposomes and LNPs are listed as adjuvants, but in this regard, they also provide another function that is, in some embodiments, co-extensive with, and in some embodiments, independent of adjuvanticity. That is, RNA, by itself and unprotected, may be degraded by the subject's RNAses. LNPs provide a means to protect the RNA by encapsulating or comprising within them an amount of the RNA of the overall composition or formulation. The LNP's effects as being in some cases an adjuvant, in other cases a delivery vehicle, and in other cases both, can be cell-dependent. To illustrate but without being bound by a particular theory, the LNP may provide adjuvanticity to peripheral blood mononuclear cells in that the RNA activates the cells but may not be expressed therein, but the LNP may provide a delivery vehicle, but not adjuvanticity, to other somatic cell types, for arguendo example, skeletal muscle cells.

In some embodiments, the pharmaceutically acceptable delivery vehicle comprises a lipid nanoparticle (LNP). In some embodiments, the LNPs encapsulate, comprise within them, consist within them, consist essentially within them, or have within them at least: 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% of the RNA. In some embodiments, the LNPs encapsulate, comprise within them, consist within them, consist essentially within them, or have within them no more than: 85.0%, 85.1%, 85.2%, 85.3%, 85.4%, 85.5%, 85.6%, 85.7%, 85.8%, 85.9%, 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100% of the RNA. In some embodiments, the LNPs encapsulate, comprise within them, consist within them, consist essentially within them, or have within them from 85% to: 86.0%, 86.1%, 86.2%, 86.3%, 86.4%, 86.5%, 86.6%, 86.7%, 86.8%, 86.9%, 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 86% to: 87.0%, 87.1%, 87.2%, 87.3%, 87.4%, 87.5%, 87.6%, 87.7%, 87.8%, 87.9%, 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 87% to 88.0%, 88.1%, 88.2%, 88.3%, 88.4%, 88.5%, 88.6%, 88.7%, 88.8%, 88.9%, 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 88% to: 89.0%, 89.1%, 89.2%, 89.3%, 89.4%, 89.5%, 89.6%, 89.7%, 89.8%, 89.9%, 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 89% to: 90.0%, 90.1%, 90.2%, 90.3%, 90.4%, 90.5%, 90.6%, 90.7%, 90.8%, 90.9%, 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 90% to: 91.0%, 91.1%, 91.2%, 91.3%, 91.4%, 91.5%, 91.6%, 91.7%, 91.8%, 91.9%, 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 91% to: 92.0%, 92.1%, 92.2%, 92.3%, 92.4%, 92.5%, 92.6%, 92.7%, 92.8%, 92.9%, 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 92% to: 93.0%, 93.1%, 93.2%, 93.3%, 93.4%, 93.5%, 93.6%, 93.7%, 93.8%, 93.9%, 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 93% to: 94.0%, 94.1%, 94.2%, 94.3%, 94.4%, 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 94% to: 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 95% to: 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 96% to: 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 97.0% to: 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 97.5% to: 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 98.0% to: 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100%; from 98.5% to: 98.6%, 98.7%, 98.8%, 98.9%, 99.0%, 99.1%, 99.2%, 99.3%, 99.4%, 99.5%, 99.6%, 99.7%, 99.8%, 99.9%, or 100%; or from: 99.0% to: 99.1%, 99.2%, 99.3%, 99.4%, 99.5%, 99.6%, 99.7%, 99.8%, 99.9%, or 100% of the SAM RNA or AAM RNA. In this regard, the foregoing percentages reference the number of RNA molecules encapsulated or comprised within the LNPs compared to the total number of RNA molecules in the composition and not the percentage of the length of any one RNA molecule being within and outside of the LNP. Also contemplated and supported are combinations of the above-noted percentages of “at least” and “no more than” with the percentages of encapsulation provided in the Examples section of this document. Also contemplated and supported are combinations of the percentages of encapsulation of this document to provide a range (i.e., the LNPs encapsulate, comprise within them, consist within them, consist essentially within them, or have within them from [the percent encapsulation of LNP formulation X₁ in the Examples] to [the percent encapsulation of LNP formulation X₂ in the Examples] wherein X₁ and X₂ represent any two exemplary LNP formulations of the Examples).

In some embodiments, 80% of the LNPs have a diameter of at least: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, 80% of the LNPs have a diameter of no more than: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, the LNPs have a diameter of at least: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm. In some embodiments, LNPs have a diameter of no more than: 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, 80% of the LNPs have a diameter from 20 nm to: 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 25 nm to: 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 30 nm to: 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 35 nm to: 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from: 40 nm to: 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 45 nm to: 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 50 nm to: 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 55 nm to: 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 60 nm to: 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 65 nm to: 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 70 nm to: 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 75 nm to: 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 80 nm to: 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; or from 85 nm to: 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

In some embodiments, the LNPs have a diameter from 20 nm to: 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 25 nm to: 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 30 nm to: 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 35 nm to: 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from: 40 nm to: 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 45 nm to: 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 50 nm to: 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 55 nm to: 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 60 nm to: 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 65 nm to: 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 70 nm to: 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 75 nm to: 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; from 80 nm to: 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm; or from 85 nm to: 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 108 nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, or 220 nm.

Taking the above-noted embodiments into account, it is contemplated and supported that any of the above-noted “diameters of no more than” and “diameters of at least” may be combined to provide an enclosed range (i.e., 80% of the LNP have a diameter from 50 nm to 80 nm). Also contemplated and supported are combinations of the above-noted “diameter no more than” or “diameter of at least” with the diameters of the LNP formulations provided in the Examples section of this document. Also contemplated and supported are combinations of the diameters of the LNP formulation section of this document to provide a range (i.e., a cation-ionizable lipid having a diameter of from [the diameter of LNP formulation X₁ in the Examples] to [the LNP formulation X₂ in the Examples] wherein X₁ and X₂ represent any two exemplary LNP formulations of the Examples.

In some embodiments, LNP has a pKa of at least: 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. In embodiments, the LNP has a pKa of no more than: 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0. In some embodiments, LNP has a pKa from 5.0 to: 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.1 to: 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.3 to: 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.4 to: 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.5 to: 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.6 to: 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.7 to: 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 5.8 to: 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.9 to: 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.0 to: 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.1 to: 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 6.2 to: 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.3 to: 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.4 to: 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.5 to: 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.6 to: 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.7 to: 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.8 to: 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.9 to: 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.0 to: 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.1 to: 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.2 to: 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.3 to: 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.4 to: 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.5 to: 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.6 to: 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.7 to: 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.9 to: 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.0 to: 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.1 to: 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.2 to: 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.3 to: 8.4, 8.5, 8.6, 8.7, 8.8, 8.9, 9.0, 9.1, 9.2, 9.3, 9.4, 9.5, 9.6, 9.7, 9.8, 9.9, or 10; or from 8.4 to: 8.5, 8.6, 8.7, 8.8, 8.9, 9.0, 9.1, 9.2, 9.3, 9.4, 9.5, 9.6, 9.7, 9.8, 9.9, or 10. Taking the above-noted embodiments into account, it is contemplated and supported that any of the above-noted “pKa of at least” and “pKa of no more than” may be combined to provide an enclosed range (i.e., a pKa of from 5.0 to 7.6 or a pKa of from 5.5 to 7.0, etc.). Also contemplated and supported are combinations of the above-noted “pKa of at least” or “pKa of no more than” with the pKa of an LNP formulation provided in the Examples section of this document. Also contemplated and supported are combinations of the pKa of the Examples section of this document to provide a range (i.e., a LNP having a pKa of from [the pKa of LNP formulation X₁ in the Examples] to [the pKa of LNP formulation X₂ in the Examples] wherein X₁ and X₂ represent any two exemplary LNP formulations of the Examples. Such embodiments are distinguishable (and combinable) with embodiments wherein the cationic lipid has a pKa by itself. In this regard, “the cationic lipid having a pKa” and “the LNP having a pKa” are distinguishable. The pKa of the LNP is influenced by the selection of cation-ionizable lipids and the mole percentages of the individual lipids in the overall formulation (i.e., the mole percentage of sterol, the mole percentage of a particular non-cationic lipid, the mole percentage of the particular cationic lipid) but to be clear the pKa of the LNP excludes the influence of the SAM RNA or AAM RNA. That is, the term “pKa of the LNP” is defined to be measured from an LNP consisting of the lipids of the LNP formulation that comprises the lipids and the SAM RNA or AAM RNA (i.e., a blank LNP wherein “blank” excludes SAM RNA or AAM RNA and any other molecules that would be delivered by the LNP). The “pKa of the LNP” is measured by the following 6-(p-toluidino)-2-naphthalenesulfonic acid (TNS) assay. TNS was suspended at 300 μM in DMSO. Following Zhang et al. “Ionization behavior of amino lipids for siRNA delivery: determination of ionization constants, SAR, and the impact of lipid pKa on cationic lipid-biomembrane interactions, LANGMUIR 27: 1907-1914 (2001), the LNPs were admixed with the 300 μM TNS in DMSO and buffered solutions containing 20 mM boric acid, 10 mM imidazole, 10 mM sodium acetate, 10 mM glycylclycine, and 25 mM NaCl and an individual pH from 3 to 10, such that each final admixture had 75 μM of cation-ionizable lipids and 6 μM of TNS and the series of admixtures provided at least one admixture within each of one pH unit within the range of pH from 3 to 10 (i.e., an admixture with said buffer having a pH within 3 (i.e., from 3.0 to 3.99), an admixture with said buffer having a pH within 4 (i.e., from 4.0 to 4.99), an admixture with said buffer having a pH within 5 (i.e., from 5.0 to 5.99), an admixture with said buffer having a pH within 6 (i.e., from 6.0 to 6.99), an admixture with said buffer having a pH within 7 (i.e., from 7.0 to 7.99), an admixture with said buffer having a pH within 8 (i.e., from 8.0 to 8.99), and an admixture with said buffer having a pH within 9 (i.e., from 9.0 to 9.99)). The fluorescence of said mixtures are then determined with an excitation wavelength of 321 nm and an emission wavelength of 445 nm. The blank-subtracted fluorescence of each admixture is then determined by subtracting a blank solution's fluorescence from each admixture's emission fluorescence. The relative fluoresence is then determined by dividing the blank-subtracted fluorescence of each admixture from the relative fluorescence of the admixture that has the highest relative fluorescence. The relative fluorescences for all the admixtures versus the pHs of the respective buffers were then regressed following the Henderson-Hasselbalch equation to obtain a line of best fit. The pKa of the LNP was determined from the pH value that has a relative fluorescence of 0.5 on the line of best fit.

### Cationic Lipids

In some embodiments, the LNP comprises lipids comprising: a cationic lipid (i.e., a cation-ionizable lipid), an optional sterol (e.g., cholesterol), an optional polymer-conjugated lipid, and an optional non-cationic lipid (“non-cationic lipid” being discrete the cationic lipid, the optional sterol (e.g., cholesterol), and the optional polymer-conjugated lipid, the “non-cationic lipid” being i.e., an optional anionic lipid or an optional neutral lipid, including zwitterionic lipids). In some embodiments, the optional neutral lipid comprises a neutral lipid zwitterionic lipid. In some embodiments, the polymer-conjugated lipid comprises a polyethylene glycol-conjugated lipid. In some embodiments, the LNP comprises a lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285, 20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, or 20150376115, U.S. Application No. 61/905,724 or Ser. No 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, or 11,285,222 (including ionizable and PEG-lipids, as referred to therein).

In some embodiments, the cationic lipid is an ionizable lipid (i.e., neutrally charged when at some pHs and positively charged when at other pHs, a.k.a. “a cation-ionizable lipid” to distinguish from a lipid which can be neutrally charged at some pHs and negatively charged at other pHs). In some embodiments, the cation-ionizable lipid has a pKa of at least: 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10. In embodiments, the cationic-ionizable lipid has a pKa of no more than: 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, or 5.0. In some embodiments, the cation-ionizable lipid has a pKa from 5.0 to: 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.1 to: 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.3 to: 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.4 to: 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.5 to: 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.6 to: 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.7 to: 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 5.8 to: 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 5.9 to: 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.0 to: 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.1 to: 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from: 6.2 to: 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.3 to: 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.4 to: 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.5 to: 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.6 to: 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.7 to: 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.8 to: 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 6.9 to: 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.0 to: 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.1 to: 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.2 to: 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.3 to: 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.4 to: 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.5 to: 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.6 to: 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.7 to: 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 7.9 to: 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.0 to: 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.1 to: 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.2 to: 8.3, 8.4, 8.5, 8.6, 8.7, 8.8, 8.9, 9.0, 9.1, 9.2, 9.3, 9.4, 9.5, 9.6, 9.7, 9.8, 9.9, or 10; from 8.3 to: 8.4, 8.5, 8.6, 8.7, 8.8, 8.9, 9.0, 9.1, 9.2, 9.3, 9.4, 9.5, 9.6, 9.7, 9.8, 9.9, or 10; or from 8.4 to: 8.5, 8.6, 8.7, 8.8, 8.9, 9.0, 9.1, 9.2, 9.3, 9.4, 9.5, 9.6, 9.7, 9.8, 9.9, or 10. Taking the above-noted embodiments into account, it is contemplated and supported that any of the above-noted “pKa of at least” and “pKa of no more than” may be combined to provide an enclosed range (i.e., a pKa of from 5.0 to 7.6 or a pKa of from 5.5 to 7.0, etc.). Also contemplated and supported are combinations of the above-noted “pKa of at least” or “pKa of no more than” with the pKa of a lipid provided in the Examples section of this document. Also contemplated and supported are combinations of the pKa of the lipids section of this document to provide a range (i.e., a cation-ionizable lipid having a pKa of from [the pKa of lipid X₁ in the Examples] to [the pKa of lipid X₂ in the Examples] wherein X₁ and X₂ represent any two exemplary lipids of the Examples. Such embodiments are distinguishable (and combinable) with embodiments wherein the LNP has a pKa as a whole (sometimes called “apparent pKa”). In this regard, “the cation-ionizable lipid having a pKa” and “the LNP having a pKa” are distinguishable. Within the meaning of “the cation-ionizable lipid having a pKa,” the pKa of the cation-ionizable lipid is determined by:
1) admixing 400 μL of 2 mM of the cationic lipid that are in 100 volume % ethanol and 800 μL of 0.3 mM of fluorescent probe toluene nitrosulphonic acid (TNS), which is in 90 volume % ethanol and 10 volume % methanol, thereby obtaining a lipid/TNS mixture;
2) admixing 7.5 μL of the lipid/TNS mixture and 242.5 μL of a first buffer comprising a sodium salt buffer comprising 20 mM sodium phosphate, 25 mM sodium citrate, 20 mM sodium acetate, and 150 mM sodium chloride, wherein the first buffer has a first pH from 4.44 to 4.52, thereby obtaining a first mixture, and dispensing 100 μL of the first mixture in a first well of a 96-well plate, which has a clear bottom;
3) admixing 7.5 μL of the lipid/TNS mixture and 242.5 μL of a second buffer comprising the sodium salt buffer, wherein the second buffer has a second pH of 5.27, thereby obtaining a second mixture, and dispensing 100 μL of the second mixture in a second well of the 96-well plate;
4) admixing 7.5 μL of the lipid/TNS mixture and 242.5 μL of a third buffer comprising the sodium salt buffer, wherein the third buffer has a third pH of from 6.15 to 6.21, thereby obtaining a third mixture, and dispensing 100 μL of the third mixture in a third well of the 96-well plate;
5) admixing 7.5 μL of the lipid/TNS mixture and 242.5 μL of a fourth buffer comprising the sodium salt buffer, wherein the fourth buffer has a fourth pH of 6.57, thereby obtaining a fourth mixture, and dispensing 100 μL of the fourth mixture in a fourth well of the 96-well plate;
6) admixing 7.5 μL of the lipid/TNS mixture and 242.5 μL of a fifth buffer comprising the sodium salt buffer, wherein the fifth buffer has a fifth pH of from 7.10 to 7.20, thereby obtaining a fifth mixture, and dispensing 100 μL of the fifth mixture in a fifth well of the 96-well plate;
7) admixing 7.5 μL of the lipid/TNS mixture and 242.5 μL of a sixth comprising the sodium salt buffer, wherein the sixth buffer has a sixth pH of from 7.72 to 7.80, thereby obtaining a sixth mixture, and dispensing 100 μL of the sixth mixture in a sixth well of the 96-well plate;
8) admixing 7.5 μL of the lipid/TNS mixture and 242.5 μL of a seventh buffer comprising the sodium salt buffer, wherein the seventh buffer has a seventh pH of from 8.27 to 8.33, thereby obtaining a seventh mixture, and dispensing 100 μL of the seventh mixture in a seventh well of the 96-well plate;
9) admixing 7.5 μL of the lipid/TNS mixture and 242.5 μL of an eighth buffer comprising the sodium salt buffer, wherein the eighth buffer has a eighth pH of from 10.47 to 11.12, thereby obtaining an eighth mixture, and dispensing 100 μL of the eighth mixture in an eighth well of the 96-well plate;
10) measuring the absolute fluorescence at a wavelength of 431 nm with an excitation wavelength of 322 nm and a cut-off below 420 nm of each of the first through eighth wells and an empty well of the 96-well plate;
11) subtracting the absolute fluorescence of the empty well from each of the absolute fluorescences of the first through the eighth wells, thereby obtaining a blank-subtracted fluorescence for each of the first through eighth mixtures;
12) normalizing each of the blank-subtracted fluorescences of the first through eighth mixtures to the blank-subtracted fluorescence of the first mixture, thereby obtaining a relative fluorescence for each of the first through eighth mixtures, the relative fluorescence of the first mixture being 1;
13) regressing by the Henderson-Hasselbalch equation, the first through eighth pHs versus the respective relative fluorescences of the first through eighth mixtures thereby obtaining a line of best fit; and
14) determining the pKa as the pH at which a relative fluorescence of 0.5 is obtained on the line of best fit.

In some embodiments, the cation-ionizable lipid comprises an amine that can be a tertiary amine and become charged depending upon the pH of the solution that the cation-ionizable lipid is in when compared to the pKa of the cation-ionizable lipid. In some embodiments, at least half of the cation-ionizable lipids are neutrally charged and the amine is a tertiary amine when the pH of the solvent that the cation-ionizable lipids are in is above the pKa; and at least half of the cation-ionizable lipids are positively charged when the pH of the solvent that the cation-ionizable lipids are in is below the pKa. In this regard, in some embodiments, but without being limited to a particular theory, it is considered that the positive charge of the ionizable lipid is distributed on the amine. Since the amine can vary in charge depending upon the pH of the solution relative to the pKa of the cation-ionizable lipid and since, without being bound a particular theory, the amine can be neutrally or positively charged when tertiary, in some embodiments, the amine is an ionizable amine.

The cation-ionizable lipid will be further described when the amine is tertiary and when the cation-ionizable lipid is neutrally charged. That is, the lipid being in a tertiary amine state and having a neutral charged is hereby described, without having to describe the cation-ionizable lipid when the lipid becomes charged. In some embodiments, the cation-ionizable lipid further comprises, in addition to the above-noted ionizable amine, a headgroup (RH) and a fatty acid tail (R^{FA1} or R^{FA2}). In some embodiments, the cation-ionizable lipid further comprises (in addition to the above-noted ionizable amine) a headgroup and at least two fatty acid tails (R^{FA1} and R^{FA2}), such as in Formula I.

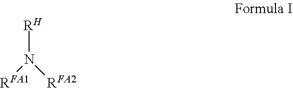

In some embodiments, the amine provides a branchpoint between the headgroup and a fatty acid tail. In some embodiments, the fatty acid tail (R^{FA}) or the at least two fatty acid tails (i.e., R^{FA1}, R^{FA2} . . . ) are immediately off of the ionizable amine. In some embodiments, the fatty acid tail comprises, or the at least two fatty acid tails comprise, a biodegradeable group (i.e., R^{BD1} or R^{BD2}), and the at least two fatty acid tails are the same or independent of one another. In some embodiments, the at least two fatty acid tails each comprise a biodegradeable group, such as in Formula II, and the biodegradeable groups are the same or independent of one another. In some embodiments, the biodegradeable group comprises, in order from the ionizable amine, —O(C═O)—, —(C═O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—, wherein X is 0, 1, or 2, and wherein R^{a} is a hydrogen or a C₁-C₁₂ alkyl. In some embodiments, the fatty acid comprises, or the at least two fatty acids comprise, a C₁-C₁₂ alkyl, a C₁-C₁₂ alkylene, or a C₁-C₁₂ alkenylene (i.e., R^{FC1} and R^{FC2}) between the amine branchpoint and the biodegradeable group), such as in Formula II. In some embodiments, the fatty acid comprises, or the two or more fatty acids comprise, distal to the ionizable amine and the biodegradeable group, a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, or a C₁₃-C₁₇ alkylene (i.e., R^{FC3} and R^{FC4}), such as in Formula II.

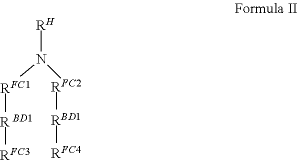

Wherein: R^{FC1} and R^{FC2} are each independently a C₁-C₁₂ alkyl, a C₁-C₁₂ alkylene, or a C₁-C₁₂ alkenylene;
R^{FC3} and R^{FC4} are each independently: a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, a C₁₃-C₁₇ alkylene;
R^{BD1} and R^{BD2} are each independently: —O(C═O)—, —(C═O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl.

In some embodiments, the C₆-C₂₄ alkyl or the C₆-C₂₄ alkylene is connected to the biodegradeable group at C₆-C₁₂, C₇-C₁₁, C₈-C₁₀, or C₉ thereof. In some embodiments, the C₆-C₂₄ alkyl or the C₆-C₂₄ alkylene of each of the at least two fatty acid tails independently comprises:

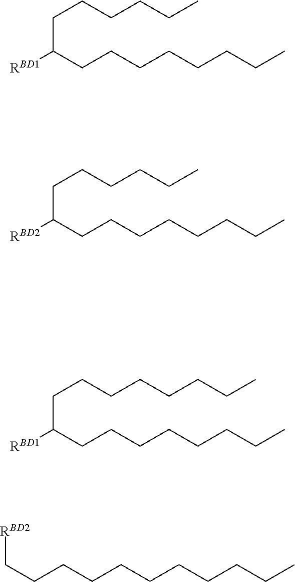

In some embodiments, the headgroup comprises, consists of, is, or has a first group (i.e., R^{H1}) and a second group (i.e., R^{H2}), wherein the first group is a C₁-C₂₄ alkyl, C₁-C₂₄ alkylene, a C₁-C₂₄ alkenylene, a C₃-C₈ cycloalkylene, or a C₃-C₈ cycloalkenylene, and the second group is H, —OH, CN, —C(═O)OR⁴, —OC(═O)OR⁴, —NR⁵C(═O)OR⁴, or OR⁵; wherein R⁴ is C₁-C₁₂ alkyl and R⁶ is H or C₁-C₆ alkyl. In some embodiments, the headgroup comprises a linear or branched form of: —(CH₂)₆OH, —(CH₂)₆OH, —(CH₂)₄OH, —(CH₂)₃OH. —(CH₂)₂OH, or —CH₂OH. In some embodiments, the cation-ionizable lipid comprises, consists of, or is [(4-Hydroxybutyl)azanediyl]di(hexane-6,1-diyl) bis(2-hexyldecanoate) or 9-Heptadecanyl 8-{(2-hydroxyethyl)[6-oxo-6-(undecyloxy)hexyl]amino}octanoate.

In some embodiments, the cation-ionizable lipid is:

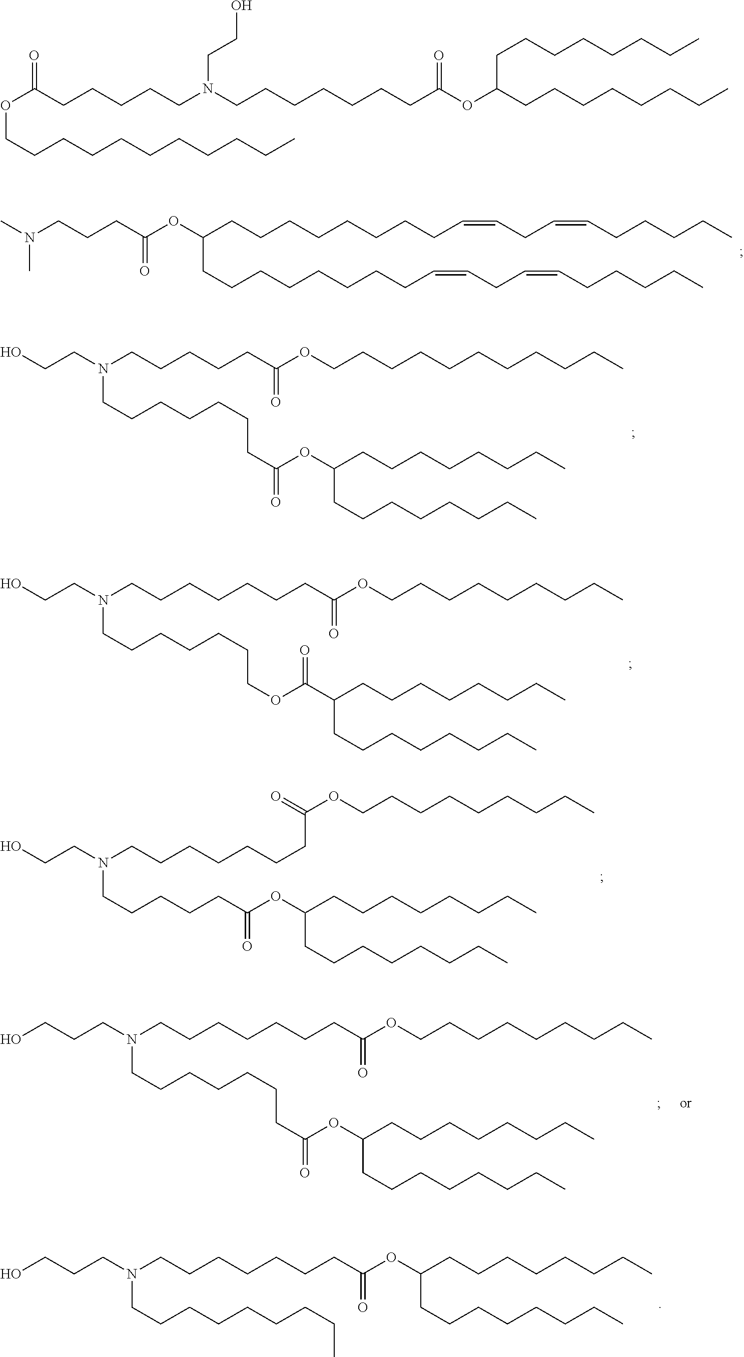

In an embodiment, the cation-ionizable lipid comprises, consists of, consists essentially of, or is RV28 having the following structure:

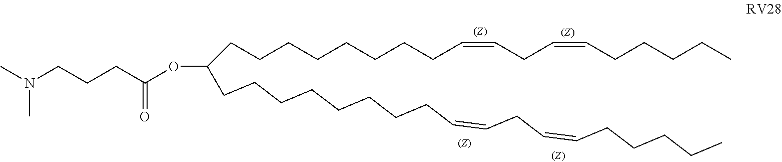 [SMILES: CCCCC/C=C\C/C=C\CCCCCCCCC(CCCCCCCC/C=C\C/C=C\CCCCC)OC(=O)CCCN(C)C]

In an embodiment, the cation-ionizable lipid comprises, consists of, consists essentially of, or is RV31 having the following structure:

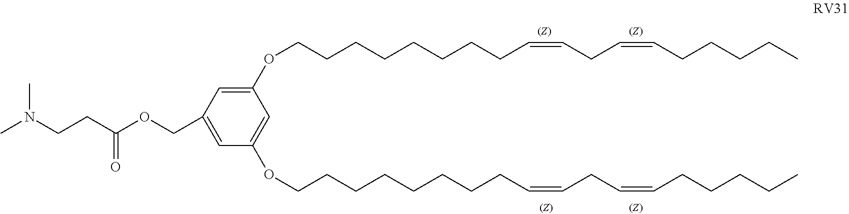 [SMILES: CCCCC/C=C\C/C=C\CCCCCCCCOc1cc(COC(=O)CCN(C)C)cc(OCCCCCCCC/C=C\C/C=C\CCCCC)c1]

In an embodiment, the cation-ionizable lipid comprises, consists of, consists essentially of, or is RV33 having the following structure:

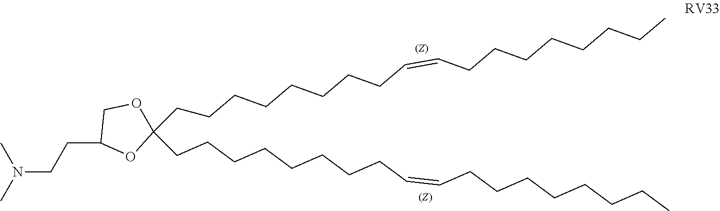 [SMILES: CCCCCCCC/C=C\CCCCCCCCC1(CCCCCCCC/C=C\CCCCCCCC)OCC(CCN(C)C)O1]

In an embodiment, the cation-ionizable lipid comprises, consists of, consists essentially of, or is RV37 having the following structure:

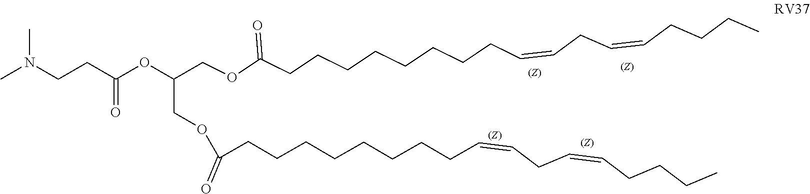 [SMILES: CCCC/C=C\C/C=C\CCCCCCCCC(=O)OCC(COC(=O)CCCCCCCC/C=C\C/C=C\CCCC)OC(=O)CCN(C)C]

In an embodiment, the cation-ionizable lipid comprises, consists of, consists essentially of, or is RV39, i.e., 2,5-bis((9Z,12Z)-octadeca-9,12-dien-1-yloxy)benzyl 4-(dimethylamino)butanoate):

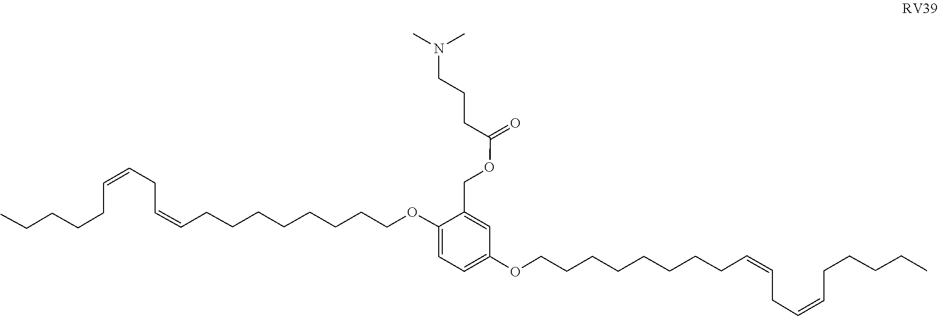 [SMILES: CCCCC/C=C\C/C=C\CCCCCCCCOc1ccc(OCCCCCCCC/C=C\C/C=C\CCCCC)c(COC(=O)CCCN(C)C)c1]

In an embodiment, the cation-ionizable lipid comprises, consists of, consists essentially of, or is RV42 having the following structure:

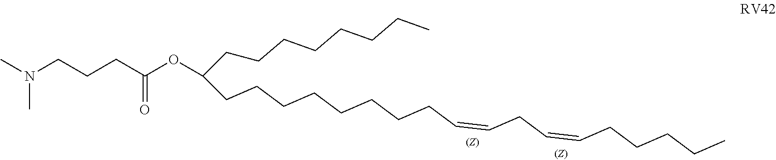 [SMILES: CCCCC/C=C\C/C=C\CCCCCCCCC(CCCCCCCC)OC(=O)CCCN(C)C]

In an embodiment, the cation-ionizable lipid comprises, consists of, consists essentially of, or is RV44 having the following structure:

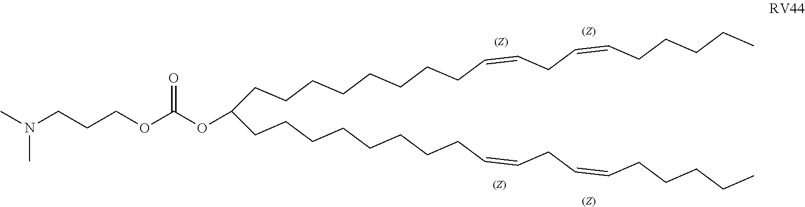 [SMILES: CCCCC/C=C\C/C=C\CCCCCCCCC(CCCCCCCC/C=C\C/C=C\CCCCC)OC(=O)OCCCN(C)C]

In an embodiment, the cation-ionizable lipid comprises, consists of, consists essentially of, or is RV73 having the following structure:

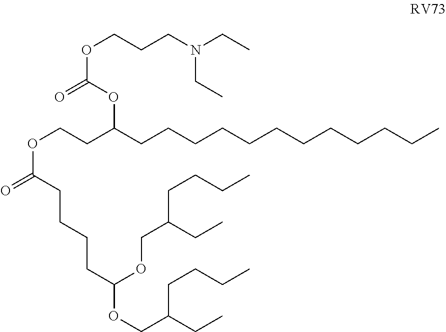 [SMILES: CCCCCCCCCCCCC(CCOC(=O)CCCCC(OCC(CC)CCCC)OCC(CC)CCCC)OC(=O)OCCCN(CC)CC]

In an embodiment, the cation-ionizable lipid comprises, consists of, consists essentially of, or is RV75 having the following structure:

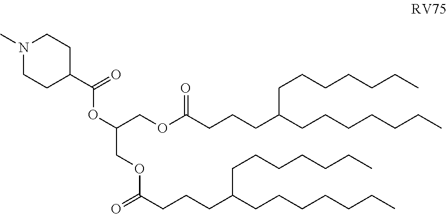 [SMILES: CCCCCCCC(CCCCCCC)CCCC(=O)OCC(COC(=O)CCCC(CCCCCCC)CCCCCCC)OC(=O)C1CCN(C)CC1]

In an embodiment, the cation-ionizable lipid comprises, consists of, consists essentially of, or is RV81 having the following structure:

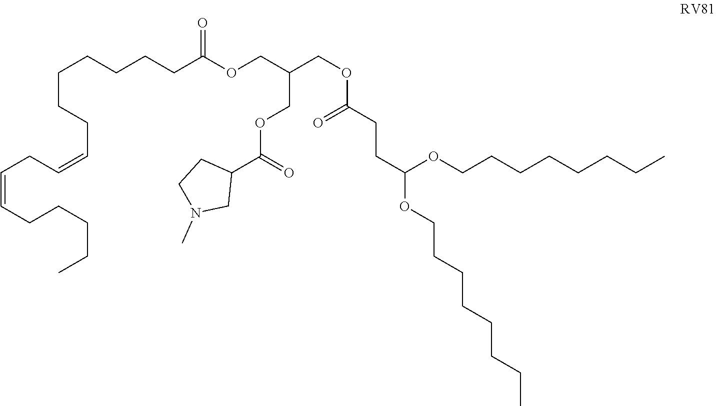 [SMILES: CCCCC/C=C\C/C=C\CCCCCCCC(=O)OCC(COC(=O)CCC(OCCCCCCCC)OCCCCCCCC)COC(=O)C1CCN(C)C1]

In an embodiment, the cation-ionizable lipid comprises, consists of, consists essentially of, or is RV84 having the following structure:

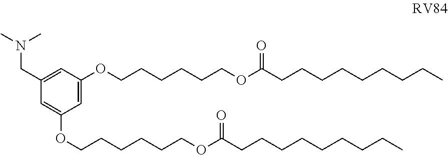 [SMILES: CCCCCCCCCC(=O)OCCCCCCOc1cc(CN(C)C)cc(OCCCCCCOC(=O)CCCCCCCCC)c1]

In an embodiment, the cation-ionizable lipid comprises, consists of, consists essentially of, or is RV85 having the following structure:

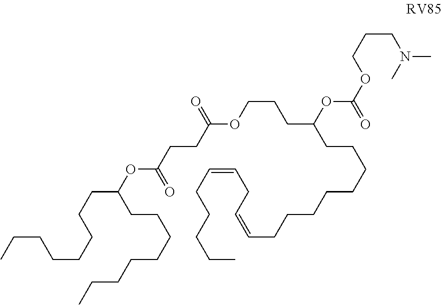 [SMILES: CCCCC/C=C\C/C=C\CCCCCCCCC(CCCOC(=O)CCC(=O)OC(CCCCCCCC)CCCCCCCC)OC(=O)OCCCN(C)C]

In an embodiment, the cation-ionizable lipid comprises, consists of, consists essentially of, or is RV86 having the following structure:

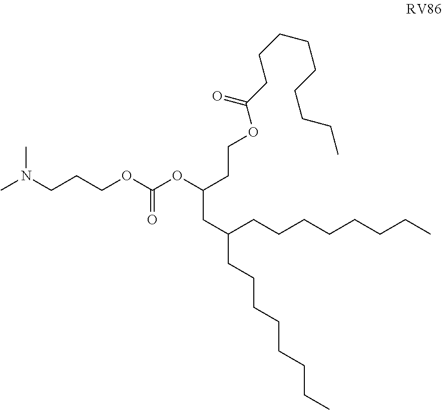 [SMILES: CCCCCCCCCC(=O)OCCC(CC(CCCCCCCC)CCCCCCCC)OC(=O)OCCCN(C)C]

In an embodiment, the cation-ionizable lipid comprises, consists of, consists essentially of, or is RV88 having the following structure:

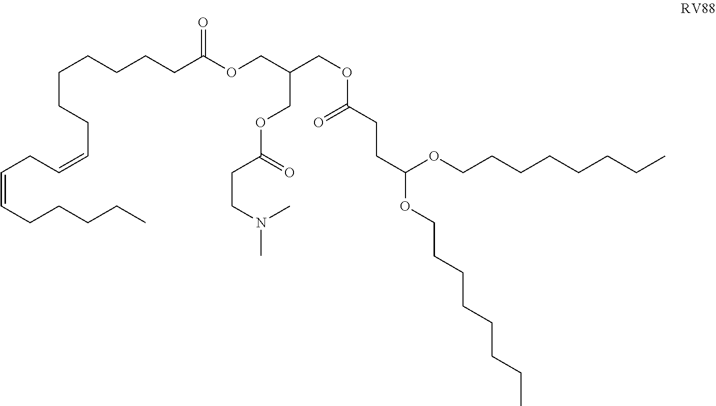 [SMILES: CCCCC/C=C\C/C=C\CCCCCCCC(=O)OCC(COC(=O)CCC(OCCCCCCCC)OCCCCCCCC)COC(=O)CCN(C)C]

In an embodiment, the cation-ionizable lipid comprises, consists of, consists essentially of, or is RV91 having the following structure:

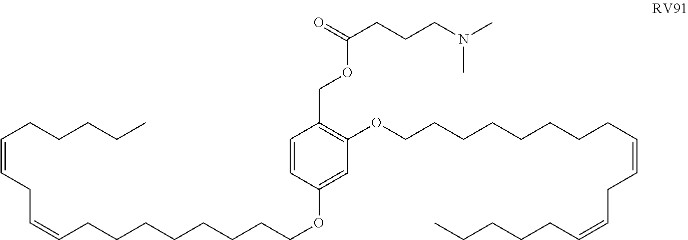 [SMILES: CCCCC/C=C\C/C=C\CCCCCCCCOc1ccc(COC(=O)CCCN(C)C)c(OCCCCCCCC/C=C\C/C=C\CCCCC)c1]

In an embodiment, the cation-ionizable lipid comprises, consists of, consists essentially of, or is RV92 having the following structure:

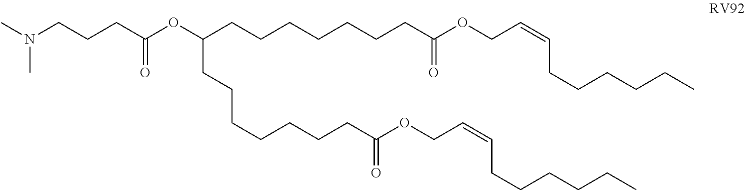 [SMILES: CCCCCC/C=C\COC(=O)CCCCCCCC(CCCCCCCC(=O)OC/C=C\CCCCCC)OC(=O)CCCN(C)C]

In an embodiment, the cation-ionizable lipid comprises, consists of, consists essentially of, or is RV93 having the following structure:

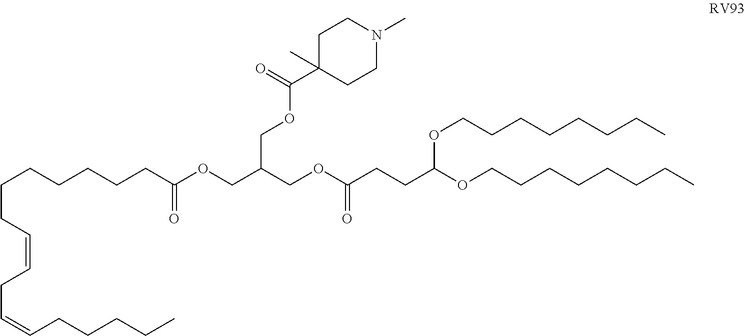 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

In an embodiment, the cation-ionizable lipid comprises, consists of, consists essentially of, or is 2-(5-((4-((1,4-dimethylpiperidine-4-carbonyl)oxy)hexadecyl)oxy)-5-oxopentyl)propane-1,3-diyl dioctanoate (RV94), having the following structure:

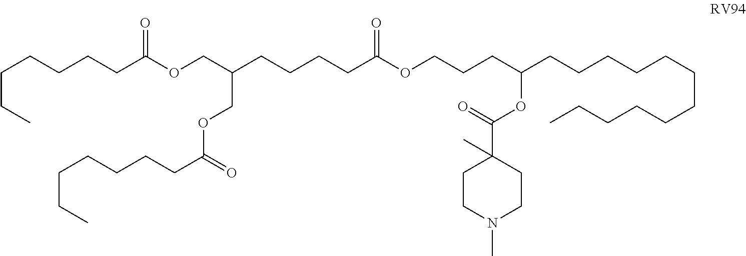 [SMILES: CCCCCCCCCCCCC(CCCOC(=O)CCCCC(COC(=O)CCCCCCC)COC(=O)CCCCCCC)OC(=O)C1(C)CCN(C)CC1]

In an embodiment, the cation-ionizable lipid comprises, consists of, consists essentially of, or is RV95 having the following structure:

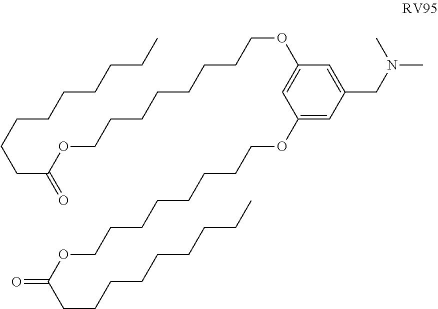 [SMILES: CCCCCCCCCC(=O)OCCCCCCCCOc1cc(CN(C)C)cc(OCCCCCCCCOC(=O)CCCCCCCCC)c1]

In an embodiment, the cation-ionizable lipid comprises, consists of, consists essentially of, or is RV96 having the following structure:

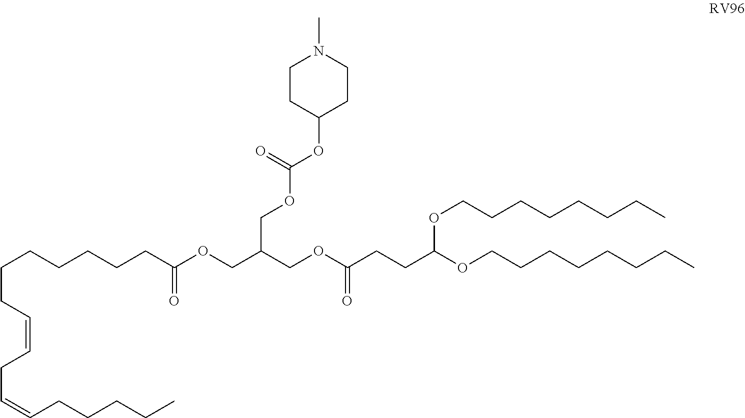 [SMILES: CCCCC/C=C\C/C=C\CCCCCCCC(=O)OCC(COC(=O)CCC(OCCCCCCCC)OCCCCCCCC)COC(=O)OC1CCN(C)CC1]

In an embodiment, the cation-ionizable lipid comprises, consists of, consists essentially of, or is RV97 having the following structure:

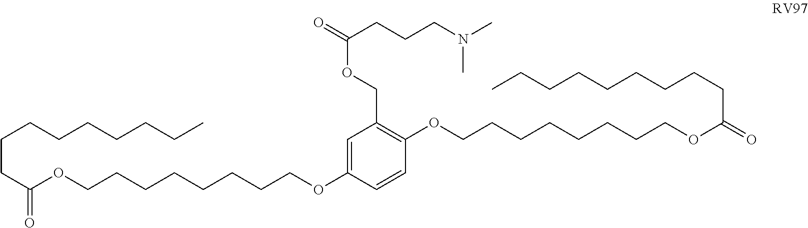 [SMILES: CCCCCCCCCC(=O)OCCCCCCCCOc1ccc(OCCCCCCCCOC(=O)CCCCCCCCC)c(COC(=O)CCCN(C)C)c1]

In an embodiment, the cation-ionizable lipid comprises, consists of, consists essentially of, or is RV99 having the following structure:

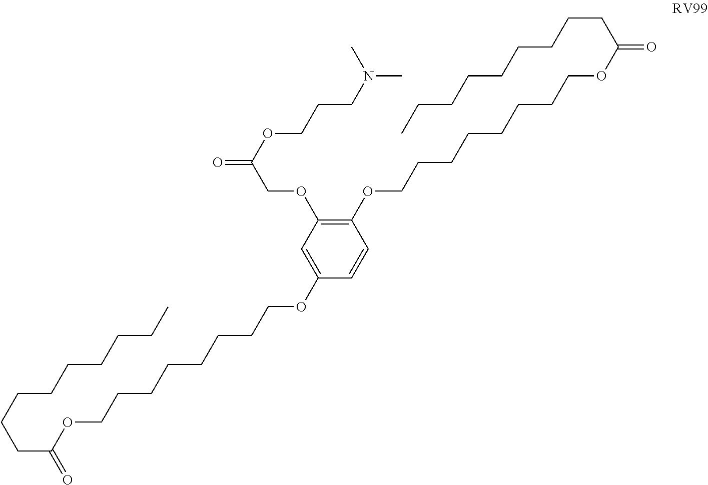 [SMILES: CCCCCCCCCC(=O)OCCCCCCCCOc1ccc(OCCCCCCCCOC(=O)CCCCCCCCC)c(OCC(=O)OCCCN(C)C)c1]

In an embodiment, the cation-ionizable lipid comprises, consists of, consists essentially of, or is RV101 having the following structure:

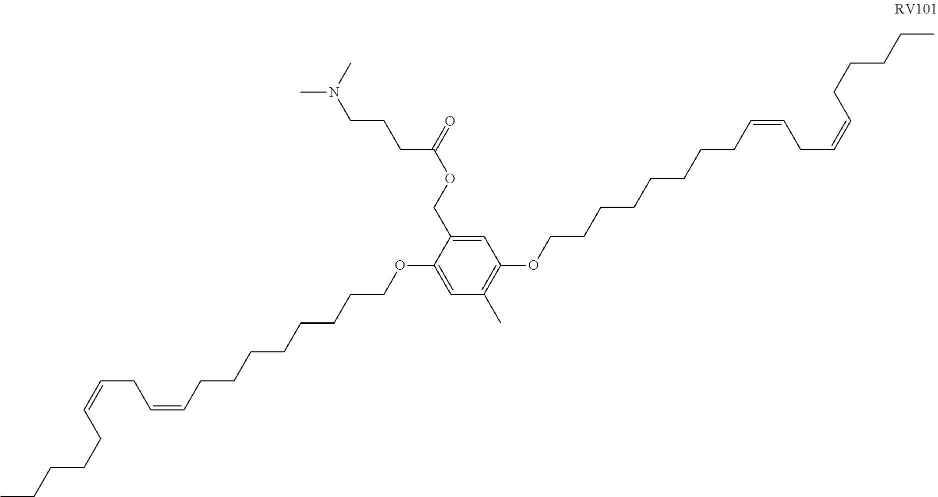 [SMILES: CCCCC/C=C\C/C=C\CCCCCCCCOc1cc(COC(=O)CCCN(C)C)c(OCCCCCCCC/C=C\C/C=C\CCCCC)cc1C]

In some embodiments, the cation-ionizable lipid comprise, consist of, consist essentially of, or are a lipid having the structure of Formula III:

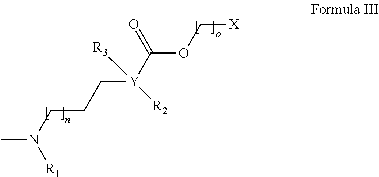

wherein n=an integer from 1 to 3 and
(i) R₁ is CH₃, R₂ and R₃ are both H, and Y is C; or
   (ii) R₁ and R₂ are collectively CH₂—CH₂ and together with the nitrogen form a five-, six-, or seven-membered heterocycloalkyl, R₃ is CH₃, and Y is C; or
   (iii) R₁ is CH₃, R₂ and R₃ are both absent, and Y is O;
wherein o is 0 or 1;
wherein X is:

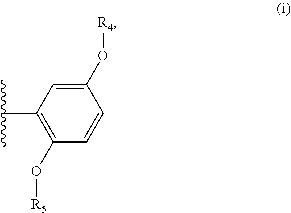

R₅, wherein R₄ and R₅ are independently a C₁₀₋₂₀ hydrocarbon chain having one or two cis alkene groups at either or both of the omega 6 and 9 positions; or
   (ii) —CH(—R₆)—R₇, wherein
(1) R₆ is —(CH₂)ₚ—O—C(O)—R₈ or —Cₚ—R₈;
(2) R₇ is —(CH₂)_{p′}—O—C(O)—R₈′ or —C_{p′}—R₈′,
(3) p and p′ are independently 0, 1, 2, 3 or 4; and
(4) R₈ and R_{8′} are independently a
(A) —C₈₋₂₀ hydrocarbon chain having one or two cis alkene groups at either or both of the omega 6 and 9 positions;
(B) —C₁₋₃—C(—O—C₆₋₁₂)—O—C₆₋₁₂ saturated or unsaturated hydrocarbon chain;
(C) —C₆₋₁₆ saturated hydrocarbon chain;
(D) —C(—C₆₋₁₆)—C₆₋₁₆ saturated or unsaturated hydrocarbon chain;
(E) —C[—C—O—C(O)—C₄₋₁₂]—C—O—C(O)—C₄₋₁₂ saturated or unsaturated hydrocarbon chain; and
(F) —C₆₋₁₆ saturated or unsaturated hydrocarbon chain.

In an embodiment, R₁ is CH₃, R₂ and R₃ are both H, and Y is C. In some embodiments, R₁ and R₂ are collectively CH₂—CH₂ and together with the nitrogen form a five-, six-, or seven-membered heterocycloalkyl, R₃ is CH₃, and Y is C. In some embodiments, R₁ is CH₃, R₂ and R₃ are both absent, and Y is O.

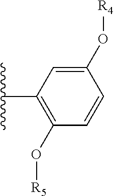

In an embodiment, X is wherein R₄ and R₅ are independently a C₁₀₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆-12 saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)_{p′}—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆-12 saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆16 saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇—C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆-12 saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆-12 saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain. In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆-12 saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain. In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆. 16)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₈, R₇—C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈″, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In some embodiments, the cation-ionizable lipid comprises a cationic lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285,20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, or 20150376115, U.S. Application No. 61/905,724 or Ser. No. 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, 11,246,933, or 11,285,222 (called ionizable lipids therein).

In some embodiments, the cation-ionizable lipid comprises a first group and two biodegradable hydrophobic tails. In some embodiments, the first group comprises a central moiety and a head group, wherein the first group is capable of being positively charged. In some embodiments, the central moiety is directly bonded to each of the two biodegradable groups. In some embodiments, the central moiety is directly bonded to the head group. In some embodiments, the central moiety is selected from a central carbon atom, a central nitrogen atom, a central heteroaryl group, and a central heterocyclic group. In some embodiments, one of the two biodegradable hydrophobic tails, or each of the two biodegradable hydrophobic tails, has the formula of: −(a C₁-C₁₂ alkyl, a C₁-C₁₂ alkylene, or a C₁-C₁₂ alkenylene)−(the biodegradable group)−(a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, a C₁₃-C₁₇ alkylene). In some embodiments, the each biodegradable group in the two biodegradable hydrophobic tails is independently selected from: —O(C═O)—, —(C═O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl. In some embodiments, in one of the two biodegradable tails, or each of the two biodegradable tails: 1) has a terminal hydrophobic chain, which is a branched alkyl group, and a terminus, 2) the branching of the branched alkyl group has an alpha-position relative to the biodegradable group, 3) 6 to 12 carbon atoms of the biodegradable hydrophobic tail separate the terminus from the biodegradable group.

In some embodiments, the cation-ionizable lipid comprises bis(2-methacryloyl)oxyethyl disulfide (DSDMA, CAS No. 36837-97-5), N,N-dioleyl-N,N-dimethylammonium chloride (DODAC), N,N-distearyl-N,N-dimethylammonium bromide (DDAB), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N, N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d][1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N1,N 16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN™ (commercially available cationic liposomes comprising DOTMA and 1,2-dioleoyl-sn-3phosphoethanolamine (DOPE), from GIBCO/BRL, Grand Island, N.Y.); LIPOFECTAMINE™ (commercially available cationic liposomes comprising N-(1-(2,3dioleyloxy)propyl)-N-(2-(sperminecarboxamido)ethyl)-N,N-dimethyl-ammonium trifluoroacetate (DOSPA), (DOPE), from GIBCO/BRL); or TRANSFECTAM™ (commercially available cationic lipids comprising dioctadecylamidoglycyl carboxyspermine (DOGS) in ethanol from Promega Corp., Madison, Wis.) or any combination of any of the foregoing. Further suitable cationic include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US Patent Application Publication No. 20150140070A1).

Representative cation-ionizable lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), 2,2-dilinoleyl-4-(2-dimethylaminoethyl)[1,3]-dioxolane (DLin-KC2-DMA); dilinoleyl-methyl-4-dimethylaminobutyrate (DLin-MC3-DMA); MC3 (US Patent Publication No. 20100324120).

In some embodiments, the LNP can comprise multilamellar vesicles (MLV); small uniflagellar vesicles (SUV); or large unilamellar vesicles (LUV). MLVs have multiple bilayers in each vesicle, forming several separate aqueous compartments. SUVs and LUVs have a single bilayer encapsulating an aqueous core. For compositions comprising LNPs with different diameters, in some embodiments: (i) at least 80% by number should have diameters in the range of 20-220 nm, (ii) the average (median) diameter (Zav, by intensity) of the population is ideally in the range of 40-200 nm, or (iii) the diameters should have a polydispersity index <0.2.

Various amphiphilic lipids can form bilayers in an aqueous environment to encapsulate a RNA-containing aqueous core as a LNP. These lipids can have an anionic, cationic, or zwitterionic hydrophilic head group. Some phospholipids are anionic whereas other are zwitterionic and others are cationic. Suitable classes of phospholipid include, but are not limited to, phosphatidylethanolamines, phosphatidylcholines, phosphatidylserines, and phosphatidyl-glycerols. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), and 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are 1,2-dipalmitoyl-sn-glycero-3-phosphocholine (DPPC), 1,2-dioleoyl-sn-glycero-3-phosphocholine (DOPC), and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

### Polyethylene Glycol-Conjugated Lipids

In some embodiments, the LNP comprises a polyethylene glycol-conjugated (PEG-conjugated) lipid. In some embodiments, the PEG-conjugated lipid comprises a polyethylene glycol (PEG) having various lengths and molecular weights. In some embodiments, 80% of the PEGs in the PEG-conjugated lipids have a molecular weight from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa. In some embodiments, 80% of the PEGs in the PEG-conjugated lipids have a molecular weight to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, 80% of the PEGs in the PEG-conjugated lipids have a molecular weight from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa.

In some embodiments, the PEGs in the PEG-conjugated lipids have a molecular weight from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa. In some embodiments, the PEGs in the PEG-conjugated lipids have a molecular weight to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, the PEGs in the PEG-conjugated lipids have a molecular weight from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa. Taking the above-noted embodiments into account, it is contemplated and supported that any of the above-noted “molecular weight from” and “molecular weight to” may be combined to provide an enclosed range (i.e., a molecular weight from 1.1 kDa to 2.4 kDa). Also contemplated and supported are combinations of the above-noted “molecular weight from” or “molecular weight to” with the molecular weight of the PEG provided in the Examples section of this document to provide an enclosed range. Also contemplated and supported are combinations of the molecular weight of the Example section of this document to provide a range (i.e., PEG having a molecular weight of from [the PEG's molecular weight of lipid X₁ in the Examples] to [the PEG's molecular weight of lipid X₂ in the Examples] wherein X₁ and X₂ represent any two exemplary PEG-conjugated lipids of the Examples.

In some embodiments, the PEGs in the PEG-conjugated lipids have a median molecular weight from: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, 3.4 kDa, 3.5 kDa, 3.6 kDa, 3.7 kDa, 3.8 kDa, 3.9 kDa, 4.0 kDa, 4.1 kDa, 4.2 kDa, 4.3 kDa, 4.4 kDa, 4.5 kDa, 4.6 kDa, 4.7 kDa, 4.8 kDa, 4.9 kDa, 5.0 kDa, 5.1 kDa, 5.2 kDa, 5.3 kDa, 5.4 kDa, 5.5 kDa, 5.6 kDa, 5.7 kDa, 5.8 kDa, 5.9 kDa, or 6.0 kDa. In some embodiments, PEGs in the PEG-conjugated lipids have a median molecular weight to: 8 kDa, 7.9 kDa, 7.8 kDa, 7.7 kDa, 7.6 kDa, 7.5 kDa, 7.4 kDa, 7.3 kDa, 7.2 kDa, 7.1 kDa, 7 kDa, 6.9 kDa, 6.8 kDa, 6.7 kDa, 6.6 kDa, 6.5 kDa, 6.4 kDa, 6.3 kDa, 6.2 kDa, 6.1 kDa, 6 kDa, 5.9 kDa, 5.8 kDa, 5.7 kDa, 5.6 kDa, 5.5 kDa, 5.4 kDa, 5.3 kDa, 5.2 kDa, 5.1 kDa, 5 kDa, 4.9 kDa, 4.8 kDa, 4.7 kDa, 4.6 kDa, 4.5 kDa, 4.4 kDa, 4.3 kDa, 4.2 kDa, 4.1 kDa, 4 kDa, 3.9 kDa, 3.8 kDa, 3.7 kDa, 3.6 kDa, 3.5 kDa, 3.4 kDa, 3.3 kDa, 3.2 kDa, 3.1 kDa, 3 kDa, 2.9 kDa, 2.8 kDa, 2.7 kDa, 2.6 kDa, 2.5 kDa, 2.4 kDa, 2.3 kDa, 2.2 kDa, 2.1 kDa, 2 kDa, 1.9 kDa, 1.8 kDa, 1.7 kDa, 1.6 kDa, 1.5 kDa, 1.4 kDa, 1.3 kDa, 1.2 kDa, or 1.1 kDa. In some embodiments, the PEGs in the PEG-conjugated lipids have a median molecular weight from 0.5 kDa to: 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.6 kDa to: 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.7 kDa to: 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.8 kDa to: 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 0.9 kDa to: 0.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.0 kDa to: 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.1 kDa to: 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.2 kDa to: 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.3 kDa to: 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.4 kDa to: 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.5 kDa to: 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.6 kDa to: 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.7 kDa to: 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.8 kDa to: 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 1.9 kDa to: 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.0 kDa to: 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.1 kDa to: 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.2 kDa to: 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.3 kDa to: 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, or 3.4 kDa; from 2.5 to: 2.6 kDa, 2.7 kDa, 2.8 kDa, 2.9 kDa, 3.0 kDa, 3.1 kDa, 3.2 kDa, 3.3 kDa, or 3.4 kDa; from 2.6 kDa to: 2.7 kDa, 2.8 kDa, 2.9 kDa, 3.0 kDa, 3.1 kDa, 3.2 kDa, 3.3 kDa, or 3.4 kDa; from 2.7 kDa to: 2.8 kDa, 2.9 kDa, 3.0 kDa, 3.1 kDa, 3.2 kDa, 3.3 kDa, or 3.4 kDa; from 2.8 kDa to: 2.9 kDa, 3.0 kDa, 3.1 kDa, 3.2 kDa, 3.3 kDa, or 3.4 kDa; from 2.9 kDa to: 3.0 kDa, 3.1 kDa, 3.2 kDa, 3.3 kDa, or 3.4 kDa; or from 3.0 kDa to: 3.1 kDa, 3.2 kDa, 3.3 kDa, or 3.4 kDa. Taking the above-noted embodiments into account, it is contemplated and supported that any of the above-noted “median molecular weight from” and “median molecular weight to” may be combined to provide an enclosed range (i.e., a median molecular weight from 1.1 kDa to 2.4 kDa). Also contemplated and supported are combinations of the above-noted “median molecular weight from” or “median molecular weight to” with the median molecular weight of the PEG provided in the Examples section of this document to provide an enclosed range. Also contemplated and supported are combinations of the median molecular weight of the Example section of this document to provide a range (i.e., PEG having a median molecular weight of from [the PEG's median molecular weight of lipid X₁ in the Examples] to [the PEG's median molecular weight of lipid X₂ in the Examples] wherein X₁ and X₂ represent any two exemplary PEG-conjugated lipids of the Examples.

In some embodiments, the PEGs in the PEG-conjugated lipids have a median molecular weight of: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, 3.4 kDa, 3.5 kDa, 3.6 kDa, 3.7 kDa, 3.8 kDa, 3.9 kDa, 4.0 kDa, 4.1 kDa, 4.2 kDa, 4.3 kDa, 4.4 kDa, 4.5 kDa, 4.6 kDa, 4.7 kDa, 4.8 kDa, 4.9 kDa, 5.0 kDa, 5.1 kDa, 5.2 kDa, 5.3 kDa, 5.4 kDa, 5.5 kDa, 5.6 kDa, 5.7 kDa, 5.8 kDa, 5.9 kDa, or 6.0 kDa.

In some embodiments, the PEG-conjugated lipid comprises 1,2-dimyristoyl-rac-glycero-3-methoxypolyethylene glycol-2000. In 1,2-dimyristoyl-rac-glycero-3-methoxypolyethylene glycol-2000, the “2000” represents the median molecular weight in Daltons of the PEG. In some embodiments, the PEG-conjugated lipid comprises 1,2-dimyristoyl-sn-glycero-2-phosphoethanolamine-N-[methoxy(polyethylene glycol)]. In some embodiments, the PEG-conjugated lipid comprises 1,2-dimyristoyl-rac-glycerol-3-methoxypolyethylene glycol.

### Non-Cationic Lipids

In some embodiments, the LNP further comprises a non-cationic lipid, which comprises an anionic lipid, a neutral lipid, or a zwitterionic lipid. In some embodiments, the neutral lipid comprises a neutral zwitterionic lipid. In some embodiments, the anionic lipid, a neutral lipid, or the zwitterionic lipid comprises a phospho-group (i.e., is a phospholipid), a choline, or a sphingolipid.

In some embodiments, the non-cationic lipid comprises 1,2-diheptadecanoyl-sn-glycero-3-phosphoethanolamine (17:0 PE), 1,2-dihexanoyl-sn-glycero-3-phosphoethanolamine (06:0 PE), 1,2-dioctanoyl-sn-glycero-3-phosphoethanolamine (08:0 PE), 1,2-didecanoyl-sn-glycero-3-phosphoethanolamine (10:0 PE), 1,2-dilauroyl-sn-glycero-3-phosphoethanolamine (12:0 PE), 1,2-dipentadecanoyl-sn-glycero-3-phosphoethanolamine (15:0 PE), 1,2-dipalmitoyl-sn-glycero-3-phosphoethanolamine (16:0 PE), 1,2-distearoyl-sn-glycero-3-phosphoethanolamine (18:0 PE), 1,2-dimyristoyl-sn-glycero-3-phosphoethanolamine (14:0 PE), 1,2-dipalmitoleoyl-sn-glycero-3-phosphoethanolamine (16:1 PE), 1,2-dioleoyl-sn-glycero-3-phosphoethanolamine (DOPE), 1,2-dielaidoyl-sn-glycero-3-phosphoethanolamine (18:1 (A9-Trans) PE), 1,2-dilinoleoyl-sn-glycero-3-phosphoethanolamine (18:2 PE), 1,2-dilinolenoyl-sn-glycero-3-phosphoethanolamine (18:3 PE), 1,2-didocosahexaenoyl-sn-glycero-3-phosphoethanolamine (22:6 PE), 1,2-diarachidonoyl-sn-glycero-3-phosphoethanolamine (20:4 PE), 1-pentadecanoyl-2-oleoyl-sn-glycero-3-phosphoethanolamine (15:0-18:1 PE), 1-palmitoyl-2-linoleoyl-sn-glycero-3-phosphoethanolamine (16:0-18:2 PE), 1-palmitoyl-2-oleoyl-sn-glycero-3-phosphoethanolamine (16:0-18:1 PE), 1-stearoyl-2-oleoyl-sn-glycero-3-phosphoethanolamine (18:0-18:1 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-hydroxy-sn-glycero-3-phosphoethanolamine (18:1 Lyso PE), 1-hydroxy-2-oleoyl-sn-glycero-3-phosphoethanolamine (2-18:1 Lyso PE), 1-palmitoyl-2-hydroxy-sn-glycero-3-phosphoethanolamine (16:0 Lyso PE), 1-tridecanoyl-sn-glycero-3-phosphoethanolamine (13:0 Lyso PE), 1-(10Z-heptadecenoyl)-sn-glycero-3-phosphoethanolamine (17:1 Lyso PE), 1-stearoyl-2-hydroxy-sn-glycero-3-phosphoethanolamine (18:0 Lyso PE), 1-myristoyl-2-hydroxy-sn-glycero-3-phosphoethanolamine (14:0 Lyso PE), L-α-phosphatidylethanolamine, 1,2-dibutyryl-sn-glycero-3-phosphocholine (04:0 PC), 1,2-dihexanoyl-sn-glycero-3-phosphocholine (DHPC), 1,2-diheptanoyl-sn-glycero-3-phosphocholine (7:0 PC), 1,2-dioctanoyl-sn-glycero-3-phosphocholine (8:0 PC), 1,2-dinonanoyl-sn-glycero-3-phosphocholine (9:0 PC), 1,2-didecanoyl-sn-glycero-3-phosphocholine (10:0 PC), 1,2-diundecanoyl-sn-glycero-3-phosphocholine (11:0 PC), 1,3-dipalmitoyl-rac-glycero-2-phosphocholine (16:0 2-PC), 1,2-dilauroyl-sn-glycero-3-phosphocholine (DLPC), 1,2-ditridecanoyl-sn-glycero-3-phosphocholine (13:0 PC), 1,2-dimyristoyl-sn-glycero-3-phosphocholine (DMPC), 1,2-dipentadecanoyl-sn-glycero-3-phosphocholine (15:0 PC), 1,2-dipalmitoyl-sn-glycero-3-phosphocholine (DPPC), 1,2-diheptadecanoyl-sn-glycero-3-phosphocholine (17:0 PC), 1,2-distearoyl-sn-glycero-3-phosphocholine (DSPC), 1,2-distearoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-divaccenoyl-sn-glycero-3-phosphocholine (18:1 (11-cis) PC), 1,2-di[(8Z)octadecenoyl]-sn-glycero-3-phosphocholine (18:1 (8-cis) PC), 1,2-dimyristoleoyl-sn-glycero-3-phosphocholine (14:1 (A9-cis) PC), 1,2-dipetroselenoyl-sn-glycero-3-phosphocholine (18:1 (A6-cis) PC), 1,2-dioleoyl-sn-glycero-3-phosphocholine (DOPC), 1,2-dielaidoyl-sn-glycero-3-phosphocholine (18:1 (A9-trans) PC), 1,2-diheptadecanoyl-sn-glycero-3-phospho-(1′-rac-glycerol), 1,2-dihexanoyl-sn-glycero-3-phospho-(1′-rac-glycerol), 1,2-dioctanoyl-sn-glycero-3-phospho-(1′-rac-glycerol), 1,2-didecanoyl-sn-glycero-3-phospho-(1′-rac-glycerol), 1,2-dilauroyl-sn-glycero-3-phospho-(1′-rac-glycerol), 1,2-dimyristoyl-sn-glycero-3-phospho-(1′-rac-glycerol), 1,2-dipentadecanoyl-sn-glycero-3-phospho-(1′-rac-glycerol), 1,2-dipalmitoyl-sn-glycero-3-phospho-(1′-rac-glycerol), 1,2-distearoyl-sn-glycero-3-phospho-(1′-rac-glycerol), 1,2-dioleoyl-sn-glycero-3-phospho-(1′-rac-glycerol), 1,2-dielaidoyl-sn-glycero-3-phospho-(1′-rac-glycerol), 1,2-dilinoleoyl-sn-glycero-3-phospho-(1′-rac-glycerol), 1,2-dilinolenoyl-sn-glycero-3-phospho-(1′-rac-glycerol), 1,2-diarachidonoyl-sn-glycero-3-[phospho-rac-(1-glycerol)], 1,2-didocosahexaenoyl-sn-glycero-3-[phospho-rac-(1-glycerol)], 1,2-dilinoleoyl-sn-glycero-3-phosphocholine (DLPC), 1,2-dilinolenoyl-sn-glycero-3-phosphocholine (18:3 (cis) PC), 1,2-dieicosenoyl-sn-glycero-3-phosphocholine (20:1 (cis) PC), 1,2-diarachidonoyl-sn-glycero-3-phosphocholine (20:4 (cis) PC), 1,2-dierucoyl-sn-glycero-3-phosphocholine (22:1 (cis) PC), 1,2-dinervonoyl-sn-glycero-3-phosphocholine (24:1 (cis) PC), 1,2-didocosahexaenoyl-sn-glycero-3-phosphocholine (22:6 (cis) PC), 1-pentadecanoyl-2-oleoyl-sn-glycero-3-phosphocholine (15:0-18:1 PC), 1-myristoyl-2-palmitoyl-sn-glycero-3-phosphocholine (14:0-16:0 PC), 1-myristoyl-2-stearoyl-sn-glycero-3-phosphocholine (14:0-18:0 PC), 1-palmitoyl-2-myristoyl-sn-glycero-3-phosphocholine (16:0-14:0 PC), 1-palmitoyl-2-stearoyl-sn-glycero-3-phosphocholine (16:0-18:0 PC), 1-palmitoyl-2-oleoyl-glycero-3-phosphocholine (POPC), 1-palmitoyl-2-linoleoyl-sn-glycero-3-phosphocholine (16:0-18:2 PC), 1-palmitoyl-2-arachidonoyl-sn-glycero-3-phosphocholine (16:0-20:4 PC), 1-palmitoyl-2-docosahexaenoyl-sn-glycero-3-phosphocholine (16:0-22:6 PC), 1-stearoyl-2-myristoyl-sn-glycero-3-phosphocholine (18:0-14:0 PC), 1-stearoyl-2-palmitoyl-sn-glycero-3-phosphocholine (18:0-16:0 PC), 1-stearoyl-2-oleoyl-sn-glycero-3-phosphocholine (18:0-18:1 PC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18:1-14:0 PC), 1-oleoyl-2-palmitoyl-sn-glycero-3-phosphocholine (18:1-16:0 PC), 1-oleoyl-2-stearoyl-sn-glycero-3-phosphocholine (18:1-18:0 PC), 1-(8Z-octadecenoyl)-2-palmitoyl-sn-glycero-3-phosphocholine (18:1(n10)-16:0 PC), 1-palmitoyl-2-acetyl-sn-glycero-3-phosphocholine (16:0-02:0 PC), 1-palmitoyl-2-[12′-(palmitoyloxy)octadecanoyl]-sn-glycero-3-phosphoholine (16:0-(12-PAHSA) PC), 1-oleoyl-2-hydroxy-sn-glycero-3-phosphocholine, 1-hexanoyl-2-hydroxy-sn-glycero-3-phosphocholine, 1-heptanoyl-2-hydroxy-sn-glycero-3-phosphocholine, 1-octanoyl-2-hydroxy-sn-glycero-3-phosphocholine, 1-nonanoyl-2-hydroxy-sn-glycero-3-phosphocholine, 1-decanoyl-2-hydroxy-sn-glycero-3-phosphocholine, 1-undecanoyl-2-hydroxy-sn-glycero-3-phosphocholine, 1-lauroyl-2-hydroxy-sn-glycero-3-phosphocholine, 1-tridecanoyl-2-hydroxy-sn-glycero-3-phosphocholine, 1-myristoyl-2-hydroxy-sn-glycero-3-phosphocholine, 1-pentadecanoyl-2-hydroxy-sn-glycero-3-phosphocholine, 1-hydroxy-2-palmitoyl-sn-glycero-3-phosphocholine, 1-palmitoyl-2-hydroxy-sn-glycero-3-phosphocholine, 1-heptadecanoyl-2-hydroxy-sn-glycero-3-phosphocholine, 1-(10Z-heptadecenoyl)-2-hydroxy-sn-glycero-3-phosphocholine, 1-hydroxy-2-oleoyl-sn-glycero-3-phosphocholine, 2-stearoyl-sn-glycero-3-phosphocholine, 1-stearoyl-2-hydroxy-sn-glycero-3-phosphocholine, 1-nonadecanoyl-2-hydroxy-sn-glycero-3-phosphocholine, 1-arachidoyl-2-hydroxy-sn-glycero-3-phosphocholine, 1-behenoyl-2-hydroxy-sn-glycero-3-phosphocholine, 1-lignoceroyl-2-hydroxy-sn-glycero-3-phosphocholine, 1-hexacosanoyl-2-hydroxy-sn-glycero-3-phosphocholine, 1,2-dimyristoyl-sn-glycero-phosphocholine (DMPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-didocosahexaenoyl-sn-glycero-3-phosphoethanolamine, 1,2-dioleoyl-sn-glycero-3-phospho-rac-(1-glycerol) sodium salt (DOPG), N-lignoceroyl-D-erythro-sphingosylphosphoethanolamine, or sphingomyelin.

### Sterols

In some embodiments, the lipid nanoparticles further comprise a sterol. In some embodiments, the sterol comprises cholesterol, cholesterol sulfate, desmosterol, stigmasterol, lanosterol, 7-dehydrocholesterol, dihydrolanosterol, symosterol, lathosteriol, 14-demethyl-lanosterol, 8(9)-dehydrocholesterol, 8(14)-dehydrocholesterol, 14-demethyl-14-dehydrolanosterol (FF-MAS), diosgenin, dehydroepiandrosterone sulfate (DHEA sulfate), dehydroepiandrosterone, sitosterol, lanosterol-95, 4,4-dimethyl(d6)-cholest-8(9), 14-dien-3β-ol (dihydro-FF-MAS-d6), 4,4-dimethyl(d6)-cholest-8(9)-en-3β-ol (dihydro T-MAS-d6), zymostenol, sitostanol, campestanol, camperstanol, 7-dehydrodesmosterol, pregnenolone, 4,4-dimethyl-cholest-8(9)-en-3β-ol (dihyrdro T-MAS), Δ5-avensterol, brassicasterol, dihydro FF-MAS, 24-methylene cholesterol, oxysterols, deuterated sterols, fluorinated sterols, sulfonated sterols, phosphorylated sterols, A-ring substituted sterols, cholest-5-ene-3β,4β-diol, 5α-cholestan-3β-ol, 4-cholesten-3-one, cholesta-8(9),24-dien-3-one, cholesta-8(9),24-dien-3-one, 2,2,3,4,4-pentadeuterio-5α-cholestan-363-ol, cholesteryl phosphocholine, cholesteryl-d7 pentadecanoate, cholesteryl-d7 palmitate, B-ring substituted sterols, cholestanol, 5β,6β-epoxy-d7, 3β-hydroxy-5-cholestene-7-one, 6α-hydroxy-5α-cholestane, cholestanol, 5α,6α-epoxy, cholest-5-en-3β,7α-diol, cholest-5-en-3β,7β-diol, cholestanol, 5α,6α-epoxy-d7, Δ5,7-cholesterol, cholesta-5,8(9)-dien-3β-ol, cholesta-5,8(14)-dien-3β-ol, 7α-hydroxy-4-cholesten-3-one, zymostenol-d7, zymostenol, 7-dehydrodesmosterol, 3β,5α-dihydroxy-cholestan-6-one, D-ring substituted sterols, 3β-hydroxy-5α-cholest-8(14)-en-15-one, 3β-hydroxy-5α-cholestane-15-one, 5α-cholest-8(14)-ene-3β,15α-diol, 5α-cholest-8(14)-ene-3β,13β,-diol, lanosterol-95, 5α-7,24-cholestadiene, 14-dehydro zymostenol, ergosta-5,7,9(11),22-tetraen-3β-ol, cholest-5-ene-3β,25-diol, cholest-(25R)-5-ene-3β,27-diol, 24(R/S),25-epoxycholesterol, 24(S),25-epoxycholesterol, 24(R/S),25-epoxycholesterol-d6, cholest-5-ene-3β,22(S)-diol, cholest-5-ene-3β,22(R)-diol, cholest-5-ene-3β,24(S)-diol, cholest-5-ene-3β,24(R)-diol, 27-hydroxy-4-cholesten-3-one, campestanol, N,N-dimethyl-3β-hydroxycholenamide, 25,27-dihydroxycholesterol, N,N-dimethyl-3β-hydroxycholenamide, 25,27-dihydroxycholesterol, 5-cholestene-3β,20α-diol, 24S,25-epoxy-5α-cholest-8(9)-en-3β-ol, 24(S/R),25-epoxylanost-8(9)-en-3β-ol, 7-keto-27-hydroxycholesterol, 7α,27-dihydroxy-4-cholesten-3-one, 7α,27-dihydroxycholesterol, 7β,27-dihydroxycholesterol, 5α,6β-dihydroxycholestanol, 7α,25-dihydroxycholesterol, 7β,25-dihydroxycholesterol, 7α,24(S)-dihydroxycholesterol, 7α,24(S)-dihydroxy-4-cholesten-3-one, 7-keto-25-hydroxycholesterol, 7α,24S,27-trihydroxycholesterol, dihydrotestosterone, testosterone, estrone, estrogen, estradiol, corticosterone, cortisol, or 24S,27-dihydroxycholesterol.

### Mole Amounts of Lipids in the LNP

In some embodiments, the lipids in the LNP have, comprise, consist of, consist essentially of, or are at least 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole % of cationic lipid. In some embodiments, the lipids in the LNP have, comprise, consist of, consist essentially of, or are no more than 80 mole %, 79 mole %, 78 mole %, 77 mole %, 76 mole %, 75 mole %, 74 mole %, 73 mole %, 72 mole %, 71 mole %, 70 mole %, 69 mole %, 68 mole %, 67 mole %, 66 mole %, 65 mole %, 64 mole %, 63 mole %, 62 mole %, 61 mole %, 60 mole %, 59 mole %, 58 mole %, 57 mole %, 56 mole %, 55 mole %, 54 mole %, 53 mole %, 52 mole %, 51 mole %, 50 mole %, 49 mole %, 48 mole %, 47 mole %, 46 mole %, 45 mole %, 44 mole %, 43 mole %, 42 mole %, 41 mole %, 40 mole %, 39 mole %, 38 mole %, 37 mole %, 36 mole %, 35 mole %, 34 mole %, 33 mole %, 32 mole %, 31 mole %, 30 mole %, 29 mole %, 28 mole %, 27 mole %, 26 mole %, 25 mole %, 24 mole %, 23 mole %, 22 mole %, 21 mole %, or 20 mole % of cationic lipid. Taking the above-noted embodiments into account, it is contemplated and supported that any of the above-noted “mole %” of “at least” and any of the above-noted “mole %” of “no more than” may be combined to provide an enclosed range (i.e., the lipids comprise from 20 mole % to 60 mole % of the cationic lipid). In some embodiments, the lipids in the LNP have, comprise, consist of, consist essentially of, or are from 20 mole % to: 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 21 mole % to: 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 22 mole % to: 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 23 mole % to: 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 24 mole % to: 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 25 mole % to: 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 26 mole % to: 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 27 mole % to: 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 28 mole % to: 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 29 mole % to: 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 30 mole % to: 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 31 mole % to: 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 32 mole % to: 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 33 mole % to: 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 34 mole % to: 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 35 mole % to: 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 36 mole % to: 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 37 mole % to: 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 38 mole % to: 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 39 mole % to: 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 40 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 41 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 42 mole % to: 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 43 mole % to: 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 44 mole % to: 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 45 mole % to: 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 46 mole % to: 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 47 mole % to: 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 48 mole % to: 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 49 mole % to: 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 50 mole % to: 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 51 mole % to: 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 52 mole % to: 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 53 mole % to: 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 54 mole % to: 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 55 mole % to: 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 56 mole % to: 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 57 mole % to: 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 58 mole % to: 59 mole %, 60 mole %, 61 mole %, 62 mole %, 63 mole %, 64 mole %, 65 mole %, 66 mole %, 67 mole %, 68 mole %, 69 mole %, or 70 mole %; from 59 mole % to: 60 mole %, 61 mole %, 62 mole %, 63 mole %, 64 mole %, 65 mole %, 66 mole %, 67 mole %, 68 mole %, 69 mole %, or 70 mole %; from 60 mole % to: 61 mole %, 62 mole %, 63 mole %, 64 mole %, 65 mole %, 66 mole %, 67 mole %, 68 mole %, 69 mole %, or 70 mole %; from 61 mole % to: 62 mole %, 63 mole %, 64 mole %, 65 mole %, 66 mole %, 67 mole %, 68 mole %, 69 mole %, or 70 mole %; from 62 mole % to: 63 mole %, 64 mole %, 65 mole %, 66 mole %, 67 mole %, 68 mole %, 69 mole %, or 70 mole %; from 63 mole % to: 64 mole %, 65 mole %, 66 mole %, 67 mole %, 68 mole %, 69 mole %, or 70 mole %; from 64 mole % to: 65 mole %, 66 mole %, 67 mole %, 68 mole %, 69 mole %, or 70 mole %; from 65 mole % to: 66 mole %, 67 mole %, 68 mole %, 69 mole %, or 70 mole %; from 66 mole % to: 67 mole %, 68 mole %, 69 mole %, or 70 mole %; from 67 mole % to: 68 mole %, 69 mole %, or 70 mole %; from 68 mole % to: 69 mole % or 70 mole %; or from 69 mole % to 70 mole % of cationic lipid. Also contemplated and supported are combinations of the above-noted “mole %” of “at least” or “mole %” of “no more than” with the mole % of the cationic lipid provided in the Examples section of this document. Also contemplated and supported are combinations of the mole % of the Example section of this document to provide a range (i.e., mole % from [the mole % of the cationic lipid of the lipids of the LNP formulation X₁ in the Examples] to [the mole % of the cationic lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least 10 mole %, 11 mole %, 12 mole %, 13 mole %, 14 mole %, 15 mole %, 16 mole %, 17 mole %, 18 mole %, 19 mole %, 20 mole %, 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole % of sterol. In some embodiments, the lipids in the LNP have, comprise, consist of, consist essentially of, or are no more than 80 mole %, 79 mole %, 78 mole %, 77 mole %, 76 mole %, 75 mole %, 74 mole %, 73 mole %, 72 mole %, 71 mole %, 70 mole %, 69 mole %, 68 mole %, 67 mole %, 66 mole %, 65 mole %, 64 mole %, 63 mole %, 62 mole %, 61 mole %, 60 mole %, 59 mole %, 58 mole %, 57 mole %, 56 mole %, 55 mole %, 54 mole %, 53 mole %, 52 mole %, 51 mole %, 50 mole %, 49 mole %, 48 mole %, 47 mole %, 46 mole %, 45 mole %, 44 mole %, 43 mole %, 42 mole %, 41 mole %, 40 mole %, 39 mole %, 38 mole %, 37 mole %, 36 mole %, 35 mole %, 34 mole %, 33 mole %, 32 mole %, 31 mole %, 30 mole %, 29 mole %, 28 mole %, 27 mole %, 26 mole %, 25 mole %, 24 mole %, 23 mole %, 22 mole %, 21 mole %, or 20 mole % of sterol. Taking the above-noted embodiments into account, it is contemplated and supported that any of the above-noted “mole %” of “at least” and any of the above-noted “mole %” of “no more than” may be combined to provide an enclosed range (i.e., the lipids comprise from 20 mole % to 60 mole % of sterol). In some embodiments, the lipids in the LNP have, comprise, consist of, consist essentially of, or are from 20 mole % to: 21 mole %, 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 21 mole % to: 22 mole %, 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 22 mole % to: 23 mole %, 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 23 mole % to: 24 mole %, 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 24 mole % to: 25 mole %, 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 25 mole % to: 26 mole %, 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 26 mole % to: 27 mole %, 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 27 mole % to: 28 mole %, 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 28 mole % to: 29 mole %, 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 29 mole % to: 30 mole %, 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 30 mole % to: 31 mole %, 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 31 mole % to: 32 mole %, 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 32 mole % to: 33 mole %, 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 33 mole % to: 34 mole %, 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 34 mole % to: 35 mole %, 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 35 mole % to: 36 mole %, 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 36 mole % to: 37 mole %, 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 37 mole % to: 38 mole %, 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 38 mole % to: 39 mole %, 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 39 mole % to: 40 mole %, 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 40 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 41 mole % to: 41 mole %, 42 mole %, 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 42 mole % to: 43 mole %, 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 43 mole % to: 44 mole %, 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 44 mole % to: 45 mole %, 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 45 mole % to: 46 mole %, 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 46 mole % to: 47 mole %, 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 47 mole % to: 48 mole %, 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 48 mole % to: 49 mole %, 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 49 mole % to: 50 mole %, 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 50 mole % to: 51 mole %, 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 51 mole % to: 52 mole %, 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 52 mole % to: 53 mole %, 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 53 mole % to: 54 mole %, 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 54 mole % to: 55 mole %, 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 55 mole % to: 56 mole %, 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 56 mole % to: 57 mole %, 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 57 mole % to: 58 mole %, 59 mole %, 60 mole %, 61 mole %, 62 mole %, 63 mole %, 64 mole %, 65 mole %, 66 mole %, 67 mole %, 68 mole %, 69 mole %, or 70 mole %; from 58 mole % to: 59 mole %, 60 mole %, 61 mole %, 62 mole %, 63 mole %, 64 mole %, 65 mole %, 66 mole %, 67 mole %, 68 mole %, 69 mole %, or 70 mole %; from 59 mole % to: 60 mole %, 61 mole %, 62 mole %, 63 mole %, 64 mole %, 65 mole %, 66 mole %, 67 mole %, 68 mole %, 69 mole %, or 70 mole %; from 60 mole % to: 61 mole %, 62 mole %, 63 mole %, 64 mole %, 65 mole %, 66 mole %, 67 mole %, 68 mole %, 69 mole %, or 70 mole %; from 61 mole % to: 62 mole %, 63 mole %, 64 mole %, 65 mole %, 66 mole %, 67 mole %, 68 mole %, 69 mole %, or 70 mole %; from 62 mole % to: 63 mole %, 64 mole %, 65 mole %, 66 mole %, 67 mole %, 68 mole %, 69 mole %, or 70 mole %; from 63 mole % to: 64 mole %, 65 mole %, 66 mole %, 67 mole %, 68 mole %, 69 mole %, or 70 mole %; from 64 mole % to: 65 mole %, 66 mole %, 67 mole %, 68 mole %, 69 mole %, or 70 mole %; from 65 mole % to: 66 mole %, 67 mole %, 68 mole %, 69 mole %, or 70 mole %; from 66 mole % to: 67 mole %, 68 mole %, 69 mole %, or 70 mole %; from 67 mole % to: 68 mole %, 69 mole %, or 70 mole %; from 68 mole % to: 69 mole % or 70 mole %; or from 69 mole % to 70 mole % of sterol. Also contemplated and supported are combinations of the above-noted “mole %” of “at least” or “mole %” of “no more than” with the mole % of sterol provided in the Examples section of this document. Also contemplated and supported are combinations of the mole % of the Example section of this document to provide a range (i.e., mole % from [the mole % of sterol of the lipids of the LNP formulation X₁ in the Examples] to [the mole % of sterol of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least 0.1 mole %, 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole % of polymer-conjugated lipid. In some embodiments, the lipids in the LNP have, comprise, consist of, consist essentially of, or are no more than 8.0 mole %, 7.9 mole %, 7.8 mole %, 7.7 mole %, 7.6 mole %, 7.5 mole %, 7.4 mole %, 7.3 mole %, 7.2 mole %, 7.1 mole %, 7.0 mole %, 6.9 mole %, 6.8 mole %, 6.7 mole %, 6.6 mole %, 6.5 mole %, 6.4 mole %, 6.3 mole %, 6.2 mole %, 6.1 mole %, 6.0 mole %, 5.9 mole %, 5.8 mole %, 5.7 mole %, 5.6 mole %, 5.5 mole %, 5.4 mole %, 5.3 mole %, 5.2 mole %, 5.1 mole %, 5.0 mole %, 4.9 mole %, 4.8 mole %, 4.7 mole %, 4.6 mole %, 4.5 mole %, 4.4 mole %, 4.3 mole %, 4.2 mole %, 4.1 mole %, 4.0 mole %, 3.9 mole %, 3.8 mole %, 3.7 mole %, 3.6 mole %, 3.5 mole %, 3.4 mole %, 3.3 mole %, 3.2 mole %, 3.1 mole %, 3.0 mole %, 2.9 mole %, 2.8 mole %, 2.7 mole %, 2.6 mole %, 2.5 mole %, 2.4 mole %, 2.3 mole %, 2.2 mole %, 2.1 mole %, 2.0 mole %, 1.9 mole %, 1.8 mole %, 1.7 mole %, 1.6 mole %, 1.5 mole %, 1.4 mole %, 1.3 mole %, 1.2 mole %, 1.1 mole %, 1.0 mole %, 0.9 mole %, 0.8 mole %, 0.7 mole %, 0.6 mole %, 0.5 mole %, 0.4 mole %, 0.3 mole %, 0.2 mole %, 0.1 mole %, or no mole % of polymer-conjugated lipid. Taking the above-noted embodiments into account, it is contemplated and supported that any of the above-noted “mole %” of “at least” and any of the above-noted “mole %” of “no more than” may be combined to provide an enclosed range (i.e., the lipids comprise from 20 mole % to 60 mole % of polymer-conjugated lipid).

In some embodiments, the lipids in the LNP have, comprise, consist of, consist essentially of, or are from 0.1 mole % to: 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.2 mole % to: 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.3 mole % to: 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.4 mole % to: 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.5 mole % to: 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.6 mole % to: 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.7 mole % to: 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.8 mole % to: 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 0.9 mole % to: 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.0 mole % to: 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.1 mole % to: 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.2 mole % to: 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.3 mole % to: 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.4 mole % to: 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.5 mole % to: 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.6 mole % to: 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.8 mole % to: 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.9 mole % to: 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.0 mole % to: 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.1 mole % to: 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.2 mole % to: 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.3 mole % to: 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.4 mole % to: 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.5 mole % to: 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.6 mole % to: 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.7 mole % to: 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.8 mole % to: 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.9 mole % to: 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.0 mole % to: 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.1 mole % to: 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.2 mole % to: 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.3 mole % to: 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.4 mole % to: 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.5 mole % to: 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.6 mole % to: 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.7 mole % to: 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.8 mole % to: 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 3.9 mole % to: 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.0 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.1 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.2 mole % to: 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.3 mole % to: 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.4 mole % to: 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.5 mole % to: 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.6 mole % to: 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.7 mole % to: 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.8 mole % to: 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 4.9 mole % to: 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.0 mole % to: 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.1 mole % to: 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.2 mole % to: 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.3 mole % to: 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.4 mole % to: 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.5 mole % to: 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.6 mole % to: 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.7 mole % to: 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.8 mole % to: 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 5.9 mole % to: 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.0 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.1 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.2 mole % to: 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.3 mole % to: 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.4 mole % to: 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.5 mole % to: 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.6 mole % to: 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.7 mole % to: 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.8 mole % to: 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.9 mole % to: 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 7.0 mole % to: 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 7.1 mole % to: 7.2 mole %, 7.3 mole %, 7.4 mole %, 7.5 mole %, 7.6 mole %, 7.7 mole %, 7.8 mole %, 7.9 mole %, or 8.0 mole %; from 7.2 mole % to: 7.3 mole %, 7.4 mole %, 7.5 mole %, 7.6 mole %, 7.7 mole %, 7.8 mole %, 7.9 mole %, or 8.0 mole %; from 7.3 mole % to: 7.4 mole %, 7.5 mole %, 7.6 mole %, 7.7 mole %, 7.8 mole %, 7.9 mole %, or 8.0 mole %; from 7.4 mole % to: 7.5 mole %, 7.6 mole %, 7.7 mole %, 7.8 mole %, 7.9 mole %, or 8.0 mole %; from 7.5 mole % to: 7.6 mole %, 7.7 mole %, 7.8 mole %, 7.9 mole %, or 8.0 mole %; from 7.6 mole % to: 7.7 mole %, 7.8 mole %, 7.9 mole %, or 8.0 mole %; from 7.7 mole % to: 7.8 mole %, 7.9 mole %, or 8.0 mole %; from 7.8 mole % to: 7.9 mole %, or 8.0 mole %; or from 7.9 mole % to 8.0 mole % of polymer-conjugated lipid. Also contemplated and supported are combinations of the above-noted “mole %” of “at least” or “mole %” of “no more than” with the mole % of polymer-conjugated lipid provided in the Examples section of this document. Also contemplated and supported are combinations of the mole % of the Example section of this document to provide a range (i.e., mole % from [the mole % of polymer-conjugated lipid of the lipids of the LNP formulation X₁ in the Examples] to [the mole % of polymer-conjugated lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least 0.1 mole %, 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole % of non-cationic lipid. In some embodiments, the lipids in the LNP have, comprise, consist of, consist essentially of, or are no more than 11.0 mole %, 10.9 mole %, 10.8 mole %, 10.7 mole %, 10.6 mole %, 10.5 mole %, 10.4 mole %, 10.3 mole %, 10.2 mole %, 10.1 mole %, 10.0 mole %, 9.9 mole %, 9.8 mole %, 9.7 mole %, 9.6 mole %, 9.5 mole %, 9.4 mole %, 9.3 mole %, 9.2 mole %, 9.1 mole %, 8.0 mole %, 7.9 mole %, 7.8 mole %, 7.7 mole %, 7.6 mole %, 7.5 mole %, 7.4 mole %, 7.3 mole %, 7.2 mole %, 7.1 mole %, 7.0 mole %, 6.9 mole %, 6.8 mole %, 6.7 mole %, 6.6 mole %, 6.5 mole %, 6.4 mole %, 6.3 mole %, 6.2 mole %, 6.1 mole %, 6.0 mole %, 5.9 mole %, 5.8 mole %, 5.7 mole %, 5.6 mole %, 5.5 mole %, 5.4 mole %, 5.3 mole %, 5.2 mole %, 5.1 mole %, 5.0 mole %, 4.9 mole %, 4.8 mole %, 4.7 mole %, 4.6 mole %, 4.5 mole %, 4.4 mole %, 4.3 mole %, 4.2 mole %, 4.1 mole %, 4.0 mole %, 3.9 mole %, 3.8 mole %, 3.7 mole %, 3.6 mole %, 3.5 mole %, 3.4 mole %, 3.3 mole %, 3.2 mole %, 3.1 mole %, 3.0 mole %, 2.9 mole %, 2.8 mole %, 2.7 mole %, 2.6 mole %, 2.5 mole %, 2.4 mole %, 2.3 mole %, 2.2 mole %, 2.1 mole %, 2.0 mole %, 1.9 mole %, 1.8 mole %, 1.7 mole %, 1.6 mole %, 1.5 mole %, 1.4 mole %, 1.3 mole %, 1.2 mole %, 1.1 mole %, 1.0 mole %, 0.9 mole %, 0.8 mole %, 0.7 mole %, 0.6 mole %, 0.5 mole %, 0.4 mole %, 0.3 mole %, 0.2 mole %, 0.1 mole %, or no mole % of non-cationic lipid. Taking the above-noted embodiments into account, it is contemplated and supported that any of the above-noted “mole %” of “at least” and any of the above-noted “mole %” of “no more than” may be combined to provide an enclosed range (i.e., the lipids comprise from 20 mole % to 60 mole % of non-cationic lipid). In some embodiments, the lipids in the LNP have, comprise, consist of, consist essentially of, or are from 0.1 mole % to: 0.2 mole %, 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.2 mole % to: 0.3 mole %, 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.3 mole % to: 0.4 mole %, 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.4 mole % to: 0.5 mole %, 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.5 mole % to: 0.6 mole %, 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.6 mole % to: 0.7 mole %, 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.7 mole % to: 0.8 mole %, 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.8 mole % to: 0.9 mole %, 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 0.9 mole % to: 1.0 mole %, 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.0 mole % to: 1.1 mole %, 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.1 mole % to: 1.2 mole %, 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.2 mole % to: 1.3 mole %, 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.3 mole % to: 1.4 mole %, 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.4 mole % to: 1.5 mole %, 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.5 mole % to: 1.6 mole %, 1.7 mole %, 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.6 mole % to: 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.7 mole % to: 1.8 mole %, 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 1.8 mole % to: 1.9 mole %, 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 1.9 mole % to: 2.0 mole %, 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.0 mole % to: 2.1 mole %, 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.1 mole % to: 2.2 mole %, 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.2 mole % to: 2.3 mole %, 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.3 mole % to: 2.4 mole %, 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 2.4 mole % to: 2.5 mole %, 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.5 mole % to: 2.6 mole %, 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.6 mole % to: 2.7 mole %, 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.7 mole % to: 2.8 mole %, 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.8 mole % to: 2.9 mole %, 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 2.9 mole % to: 3.0 mole %, 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.0 mole % to: 3.1 mole %, 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.1 mole % to: 3.2 mole %, 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.2 mole % to: 3.3 mole %, 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.3 mole % to: 3.4 mole %, 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.4 mole % to: 3.5 mole %, 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.5 mole % to: 3.6 mole %, 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.6 mole % to: 3.7 mole %, 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.7 mole % to: 3.8 mole %, 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.8 mole % to: 3.9 mole %, 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 3.9 mole % to: 4.0 mole %, 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.0 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.1 mole % to: 4.1 mole %, 4.2 mole %, 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.2 mole % to: 4.3 mole %, 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.3 mole % to: 4.4 mole %, 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.4 mole % to: 4.5 mole %, 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.5 mole % to: 4.6 mole %, 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.6 mole % to: 4.7 mole %, 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.7 mole % to: 4.8 mole %, 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.8 mole % to: 4.9 mole %, 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 4.9 mole % to: 5.0 mole %, 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.0 mole % to: 5.1 mole %, 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.1 mole % to: 5.2 mole %, 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.2 mole % to: 5.3 mole %, 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.3 mole % to: 5.4 mole %, 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.4 mole % to: 5.5 mole %, 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.5 mole % to: 5.6 mole %, 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.6 mole % to: 5.7 mole %, 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.7 mole % to: 5.8 mole %, 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.8 mole % to: 5.9 mole %, 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 5.9 mole % to: 6.0 mole %, 6.1 mole %, 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.0 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.1 mole % to: 6.2 mole %, 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.2 mole % to: 6.3 mole %, 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.3 mole % to: 6.4 mole %, 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.4 mole % to: 6.5 mole %, 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.5 mole % to: 6.6 mole %, 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.6 mole % to: 6.7 mole %, 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.7 mole % to: 6.8 mole %, 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 6.8 mole % to: 6.9 mole %, 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, or 8.0 mole %; from 6.9 mole % to: 7.0 mole %, 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.0 mole % to: 7.1 mole %, 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.1 mole % to: 7.2 mole %, 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.2 mole % to: 7.3 mole %, 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.3 mole % to: 7.4 mole %, 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.4 mole % to: 7.5 mole %, 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.5 mole % to: 7.6 mole %, 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.6 mole % to: 7.7 mole %, 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.7 mole % to: 7.8 mole %, 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.8 mole % to: 7.9 mole %, 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 7.9 mole % to: 8.0 mole %, 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.0 mole % to: 8.1 mole %, 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.1 mole % to: 8.2 mole %, 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.2 mole % to: 8.3 mole %, 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.3 mole % to: 8.4 mole %, 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.4 mole % to: 8.5 mole %, 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.5 mole % to: 8.6 mole %, 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.6 mole % to: 8.7 mole %, 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.7 mole % to: 8.8 mole %, 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.8 mole % to: 8.9 mole %, 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 8.9 mole % to: 9.0 mole %, 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.0 mole % to: 9.1 mole %, 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.1 mole % to: 9.2 mole %, 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.2 mole % to: 9.3 mole %, 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.3 mole % to: 9.4 mole %, 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.4 mole % to: 9.5 mole %, 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.5 mole % to: 9.6 mole %, 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.6 mole % to: 9.7 mole %, 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.7 mole % to: 9.8 mole %, 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.8 mole % to: 9.9 mole %, 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 9.9 mole % to: 10.0 mole %, 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.0 mole % to: 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.1 mole % to: 10.1 mole %, 10.2 mole %, 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.2 mole % to: 10.3 mole %, 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.3 mole % to: 10.4 mole %, 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.4 mole % to: 10.5 mole %, 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.5 mole % to: 10.6 mole %, 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.6 mole % to: 10.7 mole %, 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.7 mole % to: 10.8 mole %, 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.8 mole % to: 10.9 mole %, 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 10.9 mole % to: 11.0 mole %, 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.0 mole % to: 11.1 mole %, 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.1 mole % to: 11.2 mole %, 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.2 mole % to: 11.3 mole %, 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.3 mole % to: 11.4 mole %, 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.4 mole % to: 11.5 mole %, 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.5 mole % to: 11.6 mole %, 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.6 mole % to: 11.7 mole %, 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.7 mole % to: 11.8 mole %, 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; from 11.8 mole % to: 11.9 mole %, 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; or from 11.9 mole % to: 12.0 mole %, 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole %; or from 12.0 mole % to: 12.1 mole %, 12.2 mole %, 12.3 mole %, 12.4 mole %, 12.5 mole %, 12.6 mole %, 12.7 mole %, 12.8 mole %, 12.9 mole %, 12.0 mole %, 13.1 mole %, 13.2 mole %, 13.3 mole %, 13.4 mole %, 13.5 mole %, 13.6 mole %, 13.7 mole %, 13.8 mole %, 13.9 mole %, or 14.0 mole % of non-cationic lipid. Also contemplated and supported are combinations of the above-noted “mole %” of “at least” or “mole %” of “no more than” with the mole % of non-cationic lipid provided in the Examples section of this document. Also contemplated and supported are combinations of the mole % of the Example section of this document to provide a range (i.e., mole % from [the mole % of non-cationic lipid of the lipids of the LNP formulation X₁ in the Examples] to [the mole % of non-cationic lipid of the lipids of the LNP formulation X₂ in the Examples] wherein X₁ and X₂ represent any two exemplary LNP formulations of the Examples.

In some embodiments, the lipids in the LNP have, comprise, consist of, consist essentially of, or are at least a mole amount of the non-cationic lipid of 0.01-times, 0.02-times, 0.03-times, 0.04-times, 0.05-times, 0.06-times, 0.07-times, 0.08-times, 0.09-times, 0.10-times, 0.11-times, 0.12-times, 0.13-times, 0.14-times, 0.15-times, 0.16-times, 0.17-times, 0.18-times, 0.19-times, 0.20-times, 0.21-times, 0.22-times, 0.23-times, 0.24-times, 0.25-times, 0.26-times, 0.27-times, 0.28-times, 0.29-times, 0.30-times, 0.31-times, 0.32-times, 0.33-times, 0.34-times, 0.35-times, 0.36-times, 0.37-times, 0.38-times, 0.39-times, 0.40-times, 0.41-times, 0.42-times, 0.43-times, 0.44-times, 0.45-times, 0.46-times, 0.47-times, 0.48-times, 0.49-times, 0.50-times, 0.51-times, 0.52-times, 0.53-times, 0.54-times, 0.55-times, 0.56-times, 0.57-times, 0.58-times, 0.59-times, 0.60-times, 0.61-times, 0.62-times, 0.63-times, 0.64-times, 0.65-times, 0.66-times, 0.67-times, 0.68-times, 0.69-times, or 0.70-times the mole amount of the cationic lipid. In some embodiments, the lipids in the LNP have, comprise, consist of, consist essentially of, or are no more than a mole amount of the non-cationic lipid of 1.0-times, 0.99-times, 0.98-times, 0.97-times, 0.96-times, 0.95-times, 0.94-times, 0.93-times, 0.92-times, 0.91-times, 0.90-times, 0.89-times, 0.88-times, 0.87-times, 0.86-times, 0.85-times, 0.84-times, 0.83-times, 0.82-times, 0.81-times, 0.80-times, 0.79-times, 0.78-times, 0.77-times, 0.76-times, 0.75-times, 0.74-times, 0.73-times, 0.72-times, 0.71-times, 0.70-times, 0.69-times, 0.68-times, 0.67-times, 0.66-times, 0.65-times, 0.64-times, 0.63-times, 0.62-times, 0.61-times, 0.60-times, 0.59-times, 0.58-times, 0.57-times, 0.56-times, 0.55-times, 0.54-times, 0.53-times, 0.52-times, 0.51-times, 0.50-times, 0.49-times, 0.48-times, 0.47-times, 0.46-times, 0.45-times, 0.44-times, 0.43-times, 0.42-times, 0.41-times, 0.40-times, 0.39-times, 0.38-times, 0.37-times, 0.36-times, 0.35-times, 0.34-times, 0.33-times, 0.32-times, 0.31-times, 0.30-times, 0.29-times, 0.28-times, 0.27-times, 0.26-times, 0.25-times, 0.24-times, 0.23-times, 0.22-times, 0.21-times, 0.20-times, 0.19-times, 0.18-times, 0.17-times, 0.16-times, 0.15-times, 0.14-times, 0.13-times, 0.12-times, 0.11-times, 0.10-times, 0.09-times, 0.08-times, 0.07-times, 0.06-times, 0.05-times, 0.04-times, 0.03-times, 0.02-times, 0.01-times, or 0.0-times the mole amount of the cationic lipid. Taking the above-noted embodiments into account, it is contemplated and supported that any of the above-noted “at least a mole amount of the non-cationic lipid of X-times the mole amount of the cationic lipid” and “no more than a mole amount of the non-cationic lipid of Y-times the mole amount of the cationic lipid” may be combined to provide an enclosed range (i.e., the lipids in the LNP comprise a mole amount of the non-cationic lipid that is from 0.1-times to 0.2-times the amount of the cationic lipid). Also contemplated and supported are combinations of the above-noted “at least a mole amount of the non-cationic lipid of X-times the mole amount of the cationic lipid” and “no more than a mole amount of the non-cationic lipid of Y-times the mole amount of the cationic lipid” with that provided in the Examples section of this document. Also contemplated and supported are combinations of the “mole amount of the non-cationic lipid of X-times the mole amount of the cationic lipid” of the Example section of this document to provide a range (i.e., the lipids in the LNP comprise a mole amount of the non-cationic lipid that is from X₁-times to X₂-times the amount of the cationic lipid wherein X₁ and X₂ represent the mole amount of any non-cationic lipids relative to the mole amount of the cationic lipid of any two exemplary LNP formulations of the Examples.

Table 2 can be used to calculate and support the above-noted mole percentages of lipids in the LNP (which include calculating and supporting the above-noted mole percentages of at least, the above-noted mole percentages of no more than, and the above-noted mole percentages of “from” and “to”).

TABLE 2

| | | polymer- | | | | polymer- | | | | polymer- | | | | polymer- | |
|---|---|---|---|---|---|---|---|---|---|---|---|---|---|---|---|
| first | | conjugated | | first | | conjugated | | first | | conjugated | | first | | conjugated | |
| lipid | | lipid | second lipid | lipid | | lipid | second lipid | lipid | | lipid | second lipid | lipid | | lipid | second lipid |
| (i.e., | | (i.e., | (i.e anionic, | (i.e., | | (i.e., | (i.e anionic, | (i.e., | | (i.e., | (i.e anionic, | (i.e., | | (i.e., | (i.e anionic, |
| cation- | sterol | PEG- | neutral, or | cation- | sterol | PEG- | neutral, or | cation- | sterol | PEG- | neutral, or | cation- | sterol | PEG- | neutral, or |
| ionizable | (i.e., | conjugated | zwitterionic | ionizable | (i.e., | conjugated | zwitterionic | ionizable | (i.e., | conjugated | zwitterionic | ionizable | (i.e., | conjugated | zwitterionic |
| lipid) | cholesterol) | lipid) | lipid) | lipid) | cholesterol) | lipid) | lipid) | lipid) | cholesterol) | lipid) | lipid) | lipid) | cholesterol) | lipid) | lipid) |
| (mole %) | (mole %) | (mole %) | (mole %) | (mole %) | (mole %) | (mole %) | (mole %) | (mole %) | (mole %) | (mole %) | (mole %) | (mole %) | (mole %) | (mole %) | (mole %) |
| 60 | 39 | 1 | 0 | 60 | 38 | 1 | 1 | 60 | 37 | 1 | 2 | 60 | 36 | 1 | 3 |
| 59 | 40 | 1 | 0 | 59 | 39 | 1 | 1 | 59 | 38 | 1 | 2 | 59 | 37 | 1 | 3 |
| 58 | 41 | 1 | 0 | 58 | 40 | 1 | 1 | 58 | 39 | 1 | 2 | 58 | 38 | 1 | 3 |
| 57 | 42 | 1 | 0 | 57 | 41 | 1 | 1 | 57 | 40 | 1 | 2 | 57 | 39 | 1 | 3 |
| 56 | 43 | 1 | 0 | 56 | 42 | 1 | 1 | 56 | 41 | 1 | 2 | 56 | 40 | 1 | 3 |
| 55 | 44 | 1 | 0 | 55 | 43 | 1 | 1 | 55 | 42 | 1 | 2 | 55 | 41 | 1 | 3 |
| 54 | 45 | 1 | 0 | 54 | 44 | 1 | 1 | 54 | 43 | 1 | 2 | 54 | 42 | 1 | 3 |
| 53 | 46 | 1 | 0 | 53 | 45 | 1 | 1 | 53 | 44 | 1 | 2 | 53 | 43 | 1 | 3 |
| 52 | 47 | 1 | 0 | 52 | 46 | 1 | 1 | 52 | 45 | 1 | 2 | 52 | 44 | 1 | 3 |
| 51 | 48 | 1 | 0 | 51 | 47 | 1 | 1 | 51 | 46 | 1 | 2 | 51 | 45 | 1 | 3 |
| 50 | 49 | 1 | 0 | 50 | 48 | 1 | 1 | 50 | 47 | 1 | 2 | 50 | 46 | 1 | 3 |
| 49 | 50 | 1 | 0 | 49 | 49 | 1 | 1 | 49 | 48 | 1 | 2 | 49 | 47 | 1 | 3 |
| 48 | 51 | 1 | 0 | 48 | 50 | 1 | 1 | 48 | 49 | 1 | 2 | 48 | 48 | 1 | 3 |
| 47 | 52 | 1 | 0 | 47 | 51 | 1 | 1 | 47 | 50 | 1 | 2 | 47 | 49 | 1 | 3 |
| 46 | 53 | 1 | 0 | 46 | 52 | 1 | 1 | 46 | 51 | 1 | 2 | 46 | 50 | 1 | 3 |
| 45 | 54 | 1 | 0 | 45 | 53 | 1 | 1 | 45 | 52 | 1 | 2 | 45 | 51 | 1 | 3 |
| 44 | 55 | 1 | 0 | 44 | 54 | 1 | 1 | 44 | 53 | 1 | 2 | 44 | 52 | 1 | 3 |
| 43 | 56 | 1 | 0 | 43 | 55 | 1 | 1 | 43 | 54 | 1 | 2 | 43 | 53 | 1 | 3 |
| 42 | 57 | 1 | 0 | 42 | 56 | 1 | 1 | 42 | 55 | 1 | 2 | 42 | 54 | 1 | 3 |
| 41 | 58 | 1 | 0 | 41 | 57 | 1 | 1 | 41 | 56 | 1 | 2 | 41 | 55 | 1 | 3 |
| 40 | 59 | 1 | 0 | 40 | 58 | 1 | 1 | 40 | 57 | 1 | 2 | 40 | 56 | 1 | 3 |
| 39 | 60 | 1 | 0 | 39 | 59 | 1 | 1 | 39 | 58 | 1 | 2 | 39 | 57 | 1 | 3 |
| 38 | 61 | 1 | 0 | 38 | 60 | 1 | 1 | 38 | 59 | 1 | 2 | 38 | 58 | 1 | 3 |
| 37 | 62 | 1 | 0 | 37 | 61 | 1 | 1 | 37 | 60 | 1 | 2 | 37 | 59 | 1 | 3 |
| 36 | 63 | 1 | 0 | 36 | 62 | 1 | 1 | 36 | 61 | 1 | 2 | 36 | 60 | 1 | 3 |
| 35 | 64 | 1 | 0 | 35 | 63 | 1 | 1 | 35 | 62 | 1 | 2 | 35 | 61 | 1 | 3 |
| 34 | 65 | 1 | 0 | 34 | 64 | 1 | 1 | 34 | 63 | 1 | 2 | 34 | 62 | 1 | 3 |
| 33 | 66 | 1 | 0 | 33 | 65 | 1 | 1 | 33 | 64 | 1 | 2 | 33 | 63 | 1 | 3 |
| 32 | 67 | 1 | 0 | 32 | 66 | 1 | 1 | 32 | 65 | 1 | 2 | 32 | 64 | 1 | 3 |
| 31 | 68 | 1 | 0 | 31 | 67 | 1 | 1 | 31 | 66 | 1 | 2 | 31 | 65 | 1 | 3 |
| 30 | 69 | 1 | 0 | 30 | 68 | 1 | 1 | 30 | 67 | 1 | 2 | 30 | 66 | 1 | 3 |
| 60 | 38.8 | 1.2 | 0 | 60 | 39.8 | 1.2 | 1 | 60 | 36.8 | 1.2 | 2 | 60 | 35.8 | 1.2 | 3 |
| 59 | 39.8 | 1.2 | 0 | 59 | 40.8 | 1.2 | 1 | 59 | 37.8 | 1.2 | 2 | 59 | 36.8 | 1.2 | 3 |
| 58 | 40.8 | 1.2 | 0 | 58 | 41.8 | 1.2 | 1 | 58 | 38.8 | 1.2 | 2 | 58 | 37.8 | 1.2 | 3 |
| 57 | 41.8 | 1.2 | 0 | 57 | 42.8 | 1.2 | 1 | 57 | 39.8 | 1.2 | 2 | 57 | 38.8 | 1.2 | 3 |
| 56 | 42.8 | 1.2 | 0 | 56 | 43.8 | 1.2 | 1 | 56 | 40.8 | 1.2 | 2 | 56 | 39.8 | 1.2 | 3 |
| 55 | 43.8 | 1.2 | 0 | 55 | 44.8 | 1.2 | 1 | 55 | 41.8 | 1.2 | 2 | 55 | 40.8 | 1.2 | 3 |
| 54 | 44.8 | 1.2 | 0 | 54 | 45.8 | 1.2 | 1 | 54 | 42.8 | 1.2 | 2 | 54 | 41.8 | 1.2 | 3 |
| 53 | 45.8 | 1.2 | 0 | 53 | 46.8 | 1.2 | 1 | 53 | 43.8 | 1.2 | 2 | 53 | 42.8 | 1.2 | 3 |
| 52 | 46.8 | 1.2 | 0 | 52 | 47.8 | 1.2 | 1 | 52 | 44.8 | 1.2 | 2 | 52 | 43.8 | 1.2 | 3 |
| 51 | 47.8 | 1.2 | 0 | 51 | 48.8 | 1.2 | 1 | 51 | 45.8 | 1.2 | 2 | 51 | 44.8 | 1.2 | 3 |
| 50 | 48.8 | 1.2 | 0 | 50 | 49.8 | 1.2 | 1 | 50 | 46.8 | 1.2 | 2 | 50 | 45.8 | 1.2 | 3 |
| 49 | 49.8 | 1.2 | 0 | 49 | 50.8 | 1.2 | 1 | 49 | 47.8 | 1.2 | 2 | 49 | 46.8 | 1.2 | 3 |
| 48 | 50.8 | 1.2 | 0 | 48 | 51.8 | 1.2 | 1 | 48 | 48.8 | 1.2 | 2 | 48 | 47.8 | 1.2 | 3 |
| 47 | 51.8 | 1.2 | 0 | 47 | 52.8 | 1.2 | 1 | 47 | 49.8 | 1.2 | 2 | 47 | 48.8 | 1.2 | 3 |
| 46 | 52.8 | 1.2 | 0 | 46 | 53.8 | 1.2 | 1 | 46 | 50.8 | 1.2 | 2 | 46 | 49.8 | 1.2 | 3 |
| 45 | 53.8 | 1.2 | 0 | 45 | 54.8 | 1.2 | 1 | 45 | 51.8 | 1.2 | 2 | 45 | 50.8 | 1.2 | 3 |
| 44 | 54.8 | 1.2 | 0 | 44 | 55.8 | 1.2 | 1 | 44 | 52.8 | 1.2 | 2 | 44 | 51.8 | 1.2 | 3 |
| 43 | 55.8 | 1.2 | 0 | 43 | 56.8 | 1.2 | 1 | 43 | 53.8 | 1.2 | 2 | 43 | 52.8 | 1.2 | 3 |
| 42 | 56.8 | 1.2 | 0 | 42 | 57.8 | 1.2 | 1 | 42 | 54.8 | 1.2 | 2 | 42 | 53.8 | 1.2 | 3 |
| 41 | 57.8 | 1.2 | 0 | 41 | 58.8 | 1.2 | 1 | 41 | 55.8 | 1.2 | 2 | 41 | 54.8 | 1.2 | 3 |
| 40 | 58.8 | 1.2 | 0 | 40 | 59.8 | 1.2 | 1 | 40 | 56.8 | 1.2 | 2 | 40 | 55.8 | 1.2 | 3 |
| 39 | 59.8 | 1.2 | 0 | 39 | 60.8 | 1.2 | 1 | 39 | 57.8 | 1.2 | 2 | 39 | 56.8 | 1.2 | 3 |
| 38 | 60.8 | 1.2 | 0 | 38 | 61.8 | 1.2 | 1 | 38 | 58.8 | 1.2 | 2 | 38 | 57.8 | 1.2 | 3 |
| 37 | 61.8 | 1.2 | 0 | 37 | 62.8 | 1.2 | 1 | 37 | 59.8 | 1.2 | 2 | 37 | 58.8 | 1.2 | 3 |
| 36 | 62.8 | 1.2 | 0 | 36 | 63.8 | 1.2 | 1 | 36 | 60.8 | 1.2 | 2 | 36 | 59.8 | 1.2 | 3 |
| 35 | 63.8 | 1.2 | 0 | 35 | 64.8 | 1.2 | 1 | 35 | 61.8 | 1.2 | 2 | 35 | 60.8 | 1.2 | 3 |
| 34 | 64.8 | 1.2 | 0 | 34 | 65.8 | 1.2 | 1 | 34 | 62.8 | 1.2 | 2 | 34 | 61.8 | 1.2 | 3 |
| 33 | 65.8 | 1.2 | 0 | 33 | 66.8 | 1.2 | 1 | 33 | 63.8 | 1.2 | 2 | 33 | 62.8 | 1.2 | 3 |
| 32 | 66.8 | 1.2 | 0 | 32 | 67.8 | 1.2 | 1 | 32 | 64.8 | 1.2 | 2 | 32 | 63.8 | 1.2 | 3 |
| 31 | 67.8 | 1.2 | 0 | 31 | 68.8 | 1.2 | 1 | 31 | 65.8 | 1.2 | 2 | 31 | 64.8 | 1.2 | 3 |
| 30 | 68.8 | 1.2 | 0 | 30 | 67.8 | 1.2 | 1 | 30 | 66.8 | 1.2 | 2 | 30 | 65.8 | 1.2 | 3 |
| 60 | 38.6 | 1.4 | 0 | 60 | 37.6 | 1.4 | 1 | 60 | 36.6 | 1.4 | 2 | 60 | 35.6 | 1.4 | 3 |
| 59 | 39.6 | 1.4 | 0 | 59 | 38.6 | 1.4 | 1 | 59 | 37.6 | 1.4 | 2 | 59 | 36.6 | 1.4 | 3 |
| 58 | 40.6 | 1.4 | 0 | 58 | 39.6 | 1.4 | 1 | 58 | 38.6 | 1.4 | 2 | 58 | 37.6 | 1.4 | 3 |
| 57 | 41.6 | 1.4 | 0 | 57 | 40.6 | 1.4 | 1 | 57 | 39.6 | 1.4 | 2 | 57 | 38.6 | 1.4 | 3 |
| 56 | 42.6 | 1.4 | 0 | 56 | 41.6 | 1.4 | 1 | 56 | 40.6 | 1.4 | 2 | 56 | 39.6 | 1.4 | 3 |
| 55 | 43.6 | 1.4 | 0 | 55 | 42.6 | 1.4 | 1 | 55 | 41.6 | 1.4 | 2 | 55 | 40.6 | 1.4 | 3 |
| 54 | 44.6 | 1.4 | 0 | 54 | 43.6 | 1.4 | 1 | 54 | 42.6 | 1.4 | 2 | 54 | 41.6 | 1.4 | 3 |
| 53 | 45.6 | 1.4 | 0 | 53 | 44.6 | 1.4 | 1 | 53 | 43.6 | 1.4 | 2 | 53 | 42.6 | 1.4 | 3 |
| 52 | 46.6 | 1.4 | 0 | 52 | 45.6 | 1.4 | 1 | 52 | 44.6 | 1.4 | 2 | 52 | 43.6 | 1.4 | 3 |
| 51 | 47.6 | 1.4 | 0 | 51 | 46.6 | 1.4 | 1 | 51 | 45.6 | 1.4 | 2 | 51 | 44.6 | 1.4 | 3 |
| 50 | 48.6 | 1.4 | 0 | 50 | 47.6 | 1.4 | 1 | 50 | 46.6 | 1.4 | 2 | 50 | 45.6 | 1.4 | 3 |
| 49 | 49.6 | 1.4 | 0 | 49 | 48.6 | 1.4 | 1 | 49 | 47.6 | 1.4 | 2 | 49 | 46.6 | 1.4 | 3 |
| 48 | 50.6 | 1.4 | 0 | 48 | 49.6 | 1.4 | 1 | 48 | 48.6 | 1.4 | 2 | 48 | 47.6 | 1.4 | 3 |
| 47 | 51.6 | 1.4 | 0 | 47 | 50.6 | 1.4 | 1 | 47 | 49.6 | 1.4 | 2 | 47 | 48.6 | 1.4 | 3 |
| 46 | 52.6 | 1.4 | 0 | 46 | 51.6 | 1.4 | 1 | 46 | 50.6 | 1.4 | 2 | 46 | 49.6 | 1.4 | 3 |
| 45 | 53.6 | 1.4 | 0 | 45 | 52.6 | 1.4 | 1 | 45 | 51.6 | 1.4 | 2 | 45 | 50.6 | 1.4 | 3 |
| 44 | 54.6 | 1.4 | 0 | 44 | 53.6 | 1.4 | 1 | 44 | 52.6 | 1.4 | 2 | 44 | 51.6 | 1.4 | 3 |
| 43 | 55.6 | 1.4 | 0 | 43 | 54.6 | 1.4 | 1 | 43 | 53.6 | 1.4 | 2 | 43 | 52.6 | 1.4 | 3 |
| 42 | 56.6 | 1.4 | 0 | 42 | 55.6 | 1.4 | 1 | 42 | 54.6 | 1.4 | 2 | 42 | 53.6 | 1.4 | 3 |
| 41 | 57.6 | 1.4 | 0 | 41 | 56.6 | 1.4 | 1 | 41 | 55.6 | 1.4 | 2 | 41 | 54.6 | 1.4 | 3 |
| 40 | 58.6 | 1.4 | 0 | 40 | 57.6 | 1.4 | 1 | 40 | 56.6 | 1.4 | 2 | 40 | 55.6 | 1.4 | 3 |
| 39 | 59.6 | 1.4 | 0 | 39 | 58.6 | 1.4 | 1 | 39 | 57.6 | 1.4 | 2 | 39 | 56.6 | 1.4 | 3 |
| 38 | 60.6 | 1.4 | 0 | 38 | 59.6 | 1.4 | 1 | 38 | 58.6 | 1.4 | 2 | 38 | 57.6 | 1.4 | 3 |
| 37 | 61.6 | 1.4 | 0 | 37 | 60.6 | 1.4 | 1 | 37 | 59.6 | 1.4 | 2 | 37 | 58.6 | 1.4 | 3 |
| 36 | 62.6 | 1.4 | 0 | 36 | 61.6 | 1.4 | 1 | 36 | 60.6 | 1.4 | 2 | 36 | 59.6 | 1.4 | 3 |
| 35 | 63.6 | 1.4 | 0 | 35 | 62.6 | 1.4 | 1 | 35 | 61.6 | 1.4 | 2 | 35 | 60.6 | 1.4 | 3 |
| 34 | 64.6 | 1.4 | 0 | 34 | 63.6 | 1.4 | 1 | 34 | 62.6 | 1.4 | 2 | 34 | 61.6 | 1.4 | 3 |
| 33 | 65.6 | 1.4 | 0 | 33 | 64.6 | 1.4 | 1 | 33 | 63.6 | 1.4 | 2 | 33 | 62.6 | 1.4 | 3 |
| 32 | 66.6 | 1.4 | 0 | 32 | 65.6 | 1.4 | 1 | 32 | 64.6 | 1.4 | 2 | 32 | 63.6 | 1.4 | 3 |
| 31 | 67.6 | 1.4 | 0 | 31 | 66.6 | 1.4 | 1 | 31 | 65.6 | 1.4 | 2 | 31 | 64.6 | 1.4 | 3 |
| 30 | 68.6 | 1.4 | 0 | 30 | 67.6 | 1.4 | 1 | 30 | 66.6 | 1.4 | 2 | 30 | 65.6 | 1.4 | 3 |
| 60 | 38.4 | 1.6 | 0 | 60 | 37.4 | 1.6 | 1 | 60 | 36.4 | 1.6 | 2 | 60 | 35.4 | 1.6 | 3 |
| 59 | 39.4 | 1.6 | 0 | 59 | 38.4 | 1.6 | 1 | 59 | 37.4 | 1.6 | 2 | 59 | 36.4 | 1.6 | 3 |
| 58 | 40.4 | 1.6 | 0 | 58 | 39.4 | 1.6 | 1 | 58 | 38.4 | 1.6 | 2 | 58 | 37.4 | 1.6 | 3 |
| 57 | 41.4 | 1.6 | 0 | 57 | 40.4 | 1.6 | 1 | 57 | 39.4 | 1.6 | 2 | 57 | 38.4 | 1.6 | 3 |
| 56 | 42.4 | 1.6 | 0 | 56 | 41.4 | 1.6 | 1 | 56 | 40.4 | 1.6 | 2 | 56 | 39.4 | 1.6 | 3 |
| 55 | 43.4 | 1.6 | 0 | 55 | 42.4 | 1.6 | 1 | 55 | 41.4 | 1.6 | 2 | 55 | 40.4 | 1.6 | 3 |
| 54 | 44.4 | 1.6 | 0 | 54 | 43.4 | 1.6 | 1 | 54 | 42.4 | 1.6 | 2 | 54 | 41.4 | 1.6 | 3 |
| 53 | 45.4 | 1.6 | 0 | 53 | 44.4 | 1.6 | 1 | 53 | 43.4 | 1.6 | 2 | 53 | 42.4 | 1.6 | 3 |
| 52 | 46.4 | 1.6 | 0 | 52 | 45.4 | 1.6 | 1 | 52 | 44.4 | 1.6 | 2 | 52 | 43.4 | 1.6 | 3 |
| 51 | 47.4 | 1.6 | 0 | 51 | 46.4 | 1.6 | 1 | 51 | 45.4 | 1.6 | 2 | 51 | 44.4 | 1.6 | 3 |
| 50 | 48.4 | 1.6 | 0 | 50 | 47.4 | 1.6 | 1 | 50 | 46.4 | 1.6 | 2 | 50 | 45.4 | 1.6 | 3 |
| 49 | 49.4 | 1.6 | 0 | 49 | 48.4 | 1.6 | 1 | 49 | 47.4 | 1.6 | 2 | 49 | 46.4 | 1.6 | 3 |
| 48 | 50.4 | 1.6 | 0 | 48 | 49.4 | 1.6 | 1 | 48 | 48.4 | 1.6 | 2 | 48 | 47.4 | 1.6 | 3 |
| 47 | 51.4 | 1.6 | 0 | 47 | 50.4 | 1.6 | 1 | 47 | 49.4 | 1.6 | 2 | 47 | 48.4 | 1.6 | 3 |
| 46 | 52.4 | 1.6 | 0 | 46 | 51.4 | 1.6 | 1 | 46 | 50.4 | 1.6 | 2 | 46 | 49.4 | 1.6 | 3 |
| 45 | 53.4 | 1.6 | 0 | 45 | 52.4 | 1.6 | 1 | 45 | 51.4 | 1.6 | 2 | 45 | 50.4 | 1.6 | 3 |
| 44 | 54.4 | 1.6 | 0 | 44 | 53.4 | 1.6 | 1 | 44 | 52.4 | 1.6 | 2 | 44 | 51.4 | 1.6 | 3 |
| 43 | 55.4 | 1.6 | 0 | 43 | 54.4 | 1.6 | 1 | 43 | 53.4 | 1.6 | 2 | 43 | 52.4 | 1.6 | 3 |
| 42 | 56.4 | 1.6 | 0 | 42 | 55.4 | 1.6 | 1 | 42 | 54.4 | 1.6 | 2 | 42 | 53.4 | 1.6 | 3 |
| 41 | 57.4 | 1.6 | 0 | 41 | 56.4 | 1.6 | 1 | 41 | 55.4 | 1.6 | 2 | 41 | 54.4 | 1.6 | 3 |
| 40 | 58.4 | 1.6 | 0 | 40 | 57.4 | 1.6 | 1 | 40 | 56.4 | 1.6 | 2 | 40 | 55.4 | 1.6 | 3 |
| 39 | 59.4 | 1.6 | 0 | 39 | 58.4 | 1.6 | 1 | 39 | 57.4 | 1.6 | 2 | 39 | 56.4 | 1.6 | 3 |
| 38 | 60.4 | 1.6 | 0 | 38 | 59.4 | 1.6 | 1 | 38 | 58.4 | 1.6 | 2 | 38 | 57.4 | 1.6 | 3 |
| 37 | 61.4 | 1.6 | 0 | 37 | 60.4 | 1.6 | 1 | 37 | 59.4 | 1.6 | 2 | 37 | 58.4 | 1.6 | 3 |
| 36 | 62.4 | 1.6 | 0 | 36 | 61.4 | 1.6 | 1 | 36 | 60.4 | 1.6 | 2 | 36 | 59.4 | 1.6 | 3 |
| 35 | 63.4 | 1.6 | 0 | 35 | 62.4 | 1.6 | 1 | 35 | 61.4 | 1.6 | 2 | 35 | 60.4 | 1.6 | 3 |
| 34 | 64.4 | 1.6 | 0 | 34 | 63.4 | 1.6 | 1 | 34 | 62.4 | 1.6 | 2 | 34 | 61.4 | 1.6 | 3 |
| 33 | 65.4 | 1.6 | 0 | 33 | 64.4 | 1.6 | 1 | 33 | 63.4 | 1.6 | 2 | 33 | 62.4 | 1.6 | 3 |
| 32 | 66.4 | 1.6 | 0 | 32 | 65.4 | 1.6 | 1 | 32 | 64.4 | 1.6 | 2 | 32 | 63.4 | 1.6 | 3 |
| 31 | 67.4 | 1.6 | 0 | 31 | 66.4 | 1.6 | 1 | 31 | 65.4 | 1.6 | 2 | 31 | 64.4 | 1.6 | 3 |
| 30 | 68.4 | 1.6 | 0 | 30 | 67.4 | 1.6 | 1 | 30 | 66.4 | 1.6 | 2 | 30 | 65.4 | 1.6 | 3 |
| 60 | 38.2 | 1.8 | 0 | 60 | 37.2 | 1.8 | 1 | 60 | 36.2 | 1.8 | 2 | 60 | 35.2 | 1.8 | 3 |
| 59 | 39.2 | 1.8 | 0 | 59 | 38.2 | 1.8 | 1 | 59 | 37.2 | 1.8 | 2 | 59 | 36.2 | 1.8 | 3 |
| 58 | 40.2 | 1.8 | 0 | 58 | 39.2 | 1.8 | 1 | 58 | 38.2 | 1.8 | 2 | 58 | 37.2 | 1.8 | 3 |
| 57 | 41.2 | 1.8 | 0 | 57 | 40.2 | 1.8 | 1 | 57 | 39.2 | 1.8 | 2 | 57 | 38.2 | 1.8 | 3 |
| 56 | 42.2 | 1.8 | 0 | 56 | 41.2 | 1.8 | 1 | 56 | 40.2 | 1.8 | 2 | 56 | 39.2 | 1.8 | 3 |
| 55 | 43.2 | 1.8 | 0 | 55 | 42.2 | 1.8 | 1 | 55 | 41.2 | 1.8 | 2 | 55 | 40.2 | 1.8 | 3 |
| 54 | 44.2 | 1.8 | 0 | 54 | 43.2 | 1.8 | 1 | 54 | 42.2 | 1.8 | 2 | 54 | 41.2 | 1.8 | 3 |
| 53 | 45.2 | 1.8 | 0 | 53 | 44.2 | 1.8 | 1 | 53 | 43.2 | 1.8 | 2 | 53 | 42.2 | 1.8 | 3 |
| 52 | 46.2 | 1.8 | 0 | 52 | 45.2 | 1.8 | 1 | 52 | 44.2 | 1.8 | 2 | 52 | 43.2 | 1.8 | 3 |
| 51 | 47.2 | 1.8 | 0 | 51 | 46.2 | 1.8 | 1 | 51 | 45.2 | 1.8 | 2 | 51 | 44.2 | 1.8 | 3 |
| 50 | 48.2 | 1.8 | 0 | 50 | 47.2 | 1.8 | 1 | 50 | 46.2 | 1.8 | 2 | 50 | 45.2 | 1.8 | 3 |
| 49 | 49.2 | 1.8 | 0 | 49 | 48.2 | 1.8 | 1 | 49 | 47.2 | 1.8 | 2 | 49 | 46.2 | 1.8 | 3 |
| 48 | 50.2 | 1.8 | 0 | 48 | 49.2 | 1.8 | 1 | 48 | 48.2 | 1.8 | 2 | 48 | 47.2 | 1.8 | 3 |
| 47 | 51.2 | 1.8 | 0 | 47 | 50.2 | 1.8 | 1 | 47 | 49.2 | 1.8 | 2 | 47 | 48.2 | 1.8 | 3 |
| 46 | 52.2 | 1.8 | 0 | 46 | 51.2 | 1.8 | 1 | 46 | 50.2 | 1.8 | 2 | 46 | 49.2 | 1.8 | 3 |
| 45 | 53.2 | 1.8 | 0 | 45 | 52.2 | 1.8 | 1 | 45 | 51.2 | 1.8 | 2 | 45 | 50.2 | 1.8 | 3 |
| 44 | 54.2 | 1.8 | 0 | 44 | 53.2 | 1.8 | 1 | 44 | 52.2 | 1.8 | 2 | 44 | 51.2 | 1.8 | 3 |
| 43 | 55.2 | 1.8 | 0 | 43 | 54.2 | 1.8 | 1 | 43 | 53.2 | 1.8 | 2 | 43 | 52.2 | 1.8 | 3 |
| 42 | 56.2 | 1.8 | 0 | 42 | 55.2 | 1.8 | 1 | 42 | 54.2 | 1.8 | 2 | 42 | 53.2 | 1.8 | 3 |
| 41 | 57.2 | 1.8 | 0 | 41 | 56.2 | 1.8 | 1 | 41 | 55.2 | 1.8 | 2 | 41 | 54.2 | 1.8 | 3 |
| 40 | 58.2 | 1.8 | 0 | 40 | 57.2 | 1.8 | 1 | 40 | 56.2 | 1.8 | 2 | 40 | 55.2 | 1.8 | 3 |
| 39 | 59.2 | 1.8 | 0 | 39 | 58.2 | 1.8 | 1 | 39 | 57.2 | 1.8 | 2 | 39 | 56.2 | 1.8 | 3 |
| 38 | 60.2 | 1.8 | 0 | 38 | 59.2 | 1.8 | 1 | 38 | 58.2 | 1.8 | 2 | 38 | 57.2 | 1.8 | 3 |
| 37 | 61.2 | 1.8 | 0 | 37 | 60.2 | 1.8 | 1 | 37 | 59.2 | 1.8 | 2 | 37 | 58.2 | 1.8 | 3 |
| 36 | 62.2 | 1.8 | 0 | 36 | 61.2 | 1.8 | 1 | 36 | 60.2 | 1.8 | 2 | 36 | 59.2 | 1.8 | 3 |
| 35 | 63.2 | 1.8 | 0 | 35 | 62.2 | 1.8 | 1 | 35 | 61.2 | 1.8 | 2 | 35 | 60.2 | 1.8 | 3 |
| 34 | 64.2 | 1.8 | 0 | 34 | 63.2 | 1.8 | 1 | 34 | 62.2 | 1.8 | 2 | 34 | 61.2 | 1.8 | 3 |
| 33 | 65.2 | 1.8 | 0 | 33 | 64.2 | 1.8 | 1 | 33 | 63.2 | 1.8 | 2 | 33 | 62.2 | 1.8 | 3 |
| 32 | 66.2 | 1.8 | 0 | 32 | 65.2 | 1.8 | 1 | 32 | 64.2 | 1.8 | 2 | 32 | 63.2 | 1.8 | 3 |
| 31 | 67.2 | 1.8 | 0 | 31 | 66.2 | 1.8 | 1 | 31 | 65.2 | 1.8 | 2 | 31 | 64.2 | 1.8 | 3 |
| 30 | 68.2 | 1.8 | 0 | 30 | 67.2 | 1.8 | 1 | 30 | 66.2 | 1.8 | 2 | 30 | 65.2 | 1.8 | 3 |
| 60 | 38 | 2 | 0 | 60 | 37 | 2 | 1 | 60 | 36 | 2 | 2 | 60 | 35 | 2 | 3 |
| 59 | 39 | 2 | 0 | 59 | 38 | 2 | 1 | 59 | 37 | 2 | 2 | 59 | 36 | 2 | 3 |
| 58 | 40 | 2 | 0 | 58 | 39 | 2 | 1 | 58 | 38 | 2 | 2 | 58 | 37 | 2 | 3 |
| 57 | 41 | 2 | 0 | 57 | 40 | 2 | 1 | 57 | 39 | 2 | 2 | 57 | 38 | 2 | 3 |
| 56 | 42 | 2 | 0 | 56 | 41 | 2 | 1 | 56 | 40 | 2 | 2 | 56 | 39 | 2 | 3 |
| 55 | 43 | 2 | 0 | 55 | 42 | 2 | 1 | 55 | 41 | 2 | 2 | 55 | 40 | 2 | 3 |
| 54 | 44 | 2 | 0 | 54 | 43 | 2 | 1 | 54 | 42 | 2 | 2 | 54 | 41 | 2 | 3 |
| 53 | 45 | 2 | 0 | 53 | 44 | 2 | 1 | 53 | 43 | 2 | 2 | 53 | 42 | 2 | 3 |
| 52 | 46 | 2 | 0 | 52 | 45 | 2 | 1 | 52 | 44 | 2 | 2 | 52 | 43 | 2 | 3 |
| 51 | 47 | 2 | 0 | 51 | 46 | 2 | 1 | 51 | 45 | 2 | 2 | 51 | 44 | 2 | 3 |
| 50 | 48 | 2 | 0 | 50 | 47 | 2 | 1 | 50 | 46 | 2 | 2 | 50 | 45 | 2 | 3 |
| 49 | 49 | 2 | 0 | 49 | 48 | 2 | 1 | 49 | 47 | 2 | 2 | 49 | 46 | 2 | 3 |
| 48 | 50 | 2 | 0 | 48 | 49 | 2 | 1 | 48 | 48 | 2 | 2 | 48 | 47 | 2 | 2 |
| 47 | 51 | 2 | 0 | 47 | 50 | 2 | 1 | 47 | 49 | 2 | 2 | 47 | 48 | 2 | 3 |
| 46 | 52 | 2 | 0 | 46 | 51 | 2 | 1 | 46 | 50 | 2 | 2 | 46 | 49 | 2 | 3 |
| 45 | 53 | 2 | 0 | 45 | 52 | 2 | 1 | 45 | 51 | 2 | 2 | 45 | 50 | 2 | 3 |
| 44 | 54 | 2 | 0 | 44 | 53 | 2 | 1 | 44 | 52 | 2 | 2 | 44 | 51 | 2 | 3 |
| 43 | 55 | 2 | 0 | 43 | 54 | 2 | 1 | 43 | 53 | 2 | 2 | 43 | 52 | 2 | 3 |
| 42 | 56 | 2 | 0 | 42 | 55 | 2 | 1 | 42 | 54 | 2 | 2 | 42 | 53 | 2 | 3 |
| 41 | 57 | 2 | 0 | 41 | 56 | 2 | 1 | 41 | 55 | 2 | 2 | 41 | 54 | 2 | 3 |
| 40 | 58 | 2 | 0 | 40 | 57 | 2 | 1 | 40 | 56 | 2 | 2 | 40 | 55 | 2 | 3 |
| 39 | 59 | 2 | 0 | 39 | 58 | 2 | 1 | 39 | 57 | 2 | 2 | 39 | 56 | 2 | 3 |
| 38 | 60 | 2 | 0 | 38 | 59 | 2 | 1 | 38 | 58 | 2 | 2 | 38 | 57 | 2 | 3 |
| 37 | 61 | 2 | 0 | 37 | 60 | 2 | 1 | 37 | 59 | 2 | 2 | 37 | 58 | 2 | 3 |
| 36 | 62 | 2 | 0 | 36 | 61 | 2 | 1 | 36 | 60 | 2 | 2 | 36 | 59 | 2 | 3 |
| 35 | 63 | 2 | 0 | 35 | 62 | 2 | 1 | 35 | 61 | 2 | 2 | 35 | 60 | 2 | 3 |
| 34 | 64 | 2 | 0 | 34 | 63 | 2 | 1 | 34 | 62 | 2 | 2 | 34 | 61 | 2 | 3 |
| 33 | 65 | 2 | 0 | 33 | 64 | 2 | 1 | 33 | 63 | 2 | 2 | 33 | 62 | 2 | 3 |
| 32 | 66 | 2 | 0 | 32 | 65 | 2 | 1 | 32 | 64 | 2 | 2 | 32 | 63 | 2 | 3 |
| 31 | 67 | 2 | 0 | 31 | 66 | 2 | 1 | 31 | 65 | 2 | 2 | 31 | 64 | 2 | 3 |
| 30 | 68 | 2 | 0 | 30 | 67 | 2 | 1 | 30 | 66 | 2 | 2 | 30 | 65 | 2 | 3 |
| 60 | 37.8 | 2.2 | 0 | 60 | 36.8 | 2.2 | 1 | 60 | 35.8 | 2.2 | 2 | 60 | 34.8 | 2.2 | 3 |
| 59 | 38.8 | 2.2 | 0 | 59 | 37.8 | 2.2 | 1 | 59 | 36.8 | 2.2 | 2 | 59 | 35.8 | 2.2 | 3 |
| 58 | 39.8 | 2.2 | 0 | 58 | 38.8 | 2.2 | 1 | 58 | 37.8 | 2.2 | 2 | 58 | 36.8 | 2.2 | 3 |
| 57 | 40.8 | 2.2 | 0 | 57 | 39.8 | 2.2 | 1 | 57 | 38.8 | 2.2 | 2 | 57 | 37.8 | 2.2 | 3 |
| 56 | 41.8 | 2.2 | 0 | 56 | 40.8 | 2.2 | 1 | 56 | 39.8 | 2.2 | 2 | 56 | 38.8 | 2.2 | 3 |
| 55 | 42.8 | 2.2 | 0 | 55 | 41.8 | 2.2 | 1 | 55 | 40.8 | 2.2 | 2 | 55 | 39.8 | 2.2 | 3 |
| 54 | 43.8 | 2.2 | 0 | 54 | 42.8 | 2.2 | 1 | 54 | 41.8 | 2.2 | 2 | 54 | 40.8 | 2.2 | 3 |
| 53 | 44.8 | 2.2 | 0 | 53 | 43.8 | 2.2 | 1 | 53 | 42.8 | 2.2 | 2 | 53 | 41.8 | 2.2 | 3 |
| 52 | 45.8 | 2.2 | 0 | 52 | 44.8 | 2.2 | 1 | 52 | 43.8 | 2.2 | 2 | 52 | 42.8 | 2.2 | 3 |
| 51 | 46.8 | 2.2 | 0 | 51 | 45.8 | 2.2 | 1 | 51 | 44.8 | 2.2 | 2 | 51 | 43.8 | 2.2 | 3 |
| 50 | 47.8 | 2.2 | 0 | 50 | 46.8 | 2.2 | 1 | 50 | 45.8 | 2.2 | 2 | 50 | 44.8 | 2.2 | 3 |
| 49 | 48.8 | 2.2 | 0 | 49 | 47.8 | 2.2 | 1 | 49 | 46.8 | 2.2 | 2 | 49 | 45.8 | 2.2 | 3 |
| 48 | 49.8 | 2.2 | 0 | 48 | 48.8 | 2.2 | 1 | 48 | 47.8 | 2.2 | 2 | 48 | 46.8 | 2.2 | 3 |
| 47 | 50.8 | 2.2 | 0 | 47 | 49.8 | 2.2 | 1 | 47 | 48.8 | 2.2 | 2 | 47 | 47.8 | 2.2 | 3 |
| 46 | 51.8 | 2.2 | 0 | 46 | 50.8 | 2.2 | 1 | 46 | 49.8 | 2.2 | 2 | 46 | 48.8 | 2.2 | 3 |
| 45 | 52.8 | 2.2 | 0 | 45 | 51.8 | 2.2 | 1 | 45 | 50.8 | 2.2 | 2 | 45 | 49.8 | 2.2 | 3 |
| 44 | 53.8 | 2.2 | 0 | 44 | 52.8 | 2.2 | 1 | 44 | 51.8 | 2.2 | 2 | 44 | 50.8 | 2.2 | 3 |
| 43 | 54.8 | 2.2 | 0 | 43 | 53.8 | 2.2 | 1 | 43 | 52.8 | 2.2 | 2 | 43 | 51.8 | 2.2 | 3 |
| 42 | 55.8 | 2.2 | 0 | 42 | 54.8 | 2.2 | 1 | 42 | 53.8 | 2.2 | 2 | 42 | 52.8 | 2.2 | 3 |
| 41 | 56.8 | 2.2 | 0 | 41 | 55.8 | 2.2 | 1 | 41 | 54.8 | 2.2 | 2 | 41 | 53.8 | 2.2 | 3 |
| 40 | 57.8 | 2.2 | 0 | 40 | 56.8 | 2.2 | 1 | 40 | 55.8 | 2.2 | 2 | 40 | 54.8 | 2.2 | 3 |
| 39 | 58.8 | 2.2 | 0 | 39 | 57.8 | 2.2 | 1 | 39 | 56.8 | 2.2 | 2 | 39 | 55.8 | 2.2 | 3 |
| 38 | 59.8 | 2.2 | 0 | 38 | 58.8 | 2.2 | 1 | 38 | 57.8 | 2.2 | 2 | 38 | 56.8 | 2.2 | 3 |
| 37 | 60.8 | 2.2 | 0 | 37 | 59.8 | 2.2 | 1 | 37 | 58.8 | 2.2 | 2 | 37 | 57.8 | 2.2 | 3 |
| 36 | 61.8 | 2.2 | 0 | 36 | 60.8 | 2.2 | 1 | 36 | 59.8 | 2.2 | 2 | 36 | 58.8 | 2.2 | 3 |
| 35 | 62.8 | 2.2 | 0 | 35 | 61.8 | 2.2 | 1 | 35 | 60.8 | 2.2 | 2 | 35 | 59.8 | 2.2 | 3 |
| 34 | 63.8 | 2.2 | 0 | 34 | 62.8 | 2.2 | 1 | 34 | 61.8 | 2.2 | 2 | 34 | 60.8 | 2.2 | 3 |
| 33 | 64.8 | 2.2 | 0 | 33 | 63.8 | 2.2 | 1 | 33 | 62.8 | 2.2 | 2 | 33 | 61.8 | 2.2 | 3 |
| 32 | 65.8 | 2.2 | 0 | 32 | 64.8 | 2.2 | 1 | 32 | 63.8 | 2.2 | 2 | 32 | 62.8 | 2.2 | 3 |
| 31 | 66.8 | 2.2 | 0 | 31 | 65.8 | 2.2 | 1 | 31 | 64.8 | 2.2 | 2 | 31 | 63.8 | 2.2 | 3 |
| 30 | 67.8 | 2.2 | 0 | 30 | 66.8 | 2.2 | 1 | 30 | 65.8 | 2.2 | 2 | 30 | 64.8 | 2.2 | 3 |
| 60 | 37.6 | 2.4 | 0 | 60 | 36.6 | 2.4 | 1 | 60 | 35.6 | 2.4 | 2 | 60 | 34.6 | 2.4 | 3 |
| 59 | 38.6 | 2.4 | 0 | 59 | 37.6 | 2.4 | 1 | 59 | 36.6 | 2.4 | 2 | 59 | 35.6 | 2.4 | 3 |
| 58 | 39.6 | 2.4 | 0 | 58 | 38.6 | 2.4 | 1 | 58 | 37.6 | 2.4 | 2 | 58 | 36.6 | 2.4 | 3 |
| 57 | 40.6 | 2.4 | 0 | 57 | 39.6 | 2.4 | 1 | 57 | 38.6 | 2.4 | 2 | 57 | 37.6 | 2.4 | 3 |
| 56 | 41.6 | 2.4 | 0 | 56 | 40.6 | 2.4 | 1 | 56 | 39.6 | 2.4 | 2 | 56 | 38.6 | 2.4 | 3 |
| 55 | 42.6 | 2.4 | 0 | 55 | 41.6 | 2.4 | 1 | 55 | 40.6 | 2.4 | 2 | 55 | 39.6 | 2.4 | 3 |
| 54 | 43.6 | 2.4 | 0 | 54 | 42.6 | 2.4 | 1 | 54 | 41.6 | 2.4 | 2 | 54 | 40.6 | 2.4 | 3 |
| 53 | 44.6 | 2.4 | 0 | 53 | 43.6 | 2.4 | 1 | 53 | 42.6 | 2.4 | 2 | 53 | 41.6 | 2.4 | 3 |
| 52 | 45.6 | 2.4 | 0 | 52 | 44.6 | 2.4 | 1 | 52 | 43.6 | 2.4 | 2 | 52 | 42.6 | 2.4 | 3 |
| 51 | 46.6 | 2.4 | 0 | 51 | 45.6 | 2.4 | 1 | 51 | 44.6 | 2.4 | 2 | 51 | 43.6 | 2.4 | 3 |
| 50 | 47.6 | 2.4 | 0 | 50 | 46.6 | 2.4 | 1 | 50 | 45.6 | 2.4 | 2 | 50 | 44.6 | 2.4 | 3 |
| 49 | 48.6 | 2.4 | 0 | 49 | 47.6 | 2.4 | 1 | 49 | 46.6 | 2.4 | 2 | 49 | 45.6 | 2.4 | 3 |
| 48 | 49.6 | 2.4 | 0 | 48 | 48.6 | 2.4 | 1 | 48 | 47.6 | 2.4 | 2 | 48 | 46.6 | 2.4 | 3 |
| 47 | 50.6 | 2.4 | 0 | 47 | 49.6 | 2.4 | 1 | 47 | 48.6 | 2.4 | 2 | 47 | 47.6 | 2.4 | 3 |
| 46 | 51.6 | 2.4 | 0 | 46 | 50.6 | 2.4 | 1 | 46 | 49.6 | 2.4 | 2 | 46 | 48.6 | 2.4 | 3 |
| 45 | 52.6 | 2.4 | 0 | 45 | 51.6 | 2.4 | 1 | 45 | 50.6 | 2.4 | 2 | 45 | 49.6 | 2.4 | 3 |
| 44 | 53.6 | 2.4 | 0 | 44 | 52.6 | 2.4 | 1 | 44 | 51.6 | 2.4 | 2 | 44 | 50.6 | 2.4 | 3 |
| 43 | 54.6 | 2.4 | 0 | 43 | 53.6 | 2.4 | 1 | 43 | 52.6 | 2.4 | 2 | 43 | 51.6 | 2.4 | 3 |
| 42 | 55.6 | 2.4 | 0 | 42 | 54.6 | 2.4 | 1 | 42 | 53.6 | 2.4 | 2 | 42 | 52.6 | 2.4 | 3 |
| 41 | 56.6 | 2.4 | 0 | 41 | 55.6 | 2.4 | 1 | 41 | 54.6 | 2.4 | 2 | 41 | 53.6 | 2.4 | 3 |
| 40 | 57.6 | 2.4 | 0 | 40 | 56.6 | 2.4 | 1 | 40 | 55.6 | 2.4 | 2 | 40 | 54.6 | 2.4 | 3 |
| 39 | 58.6 | 2.4 | 0 | 39 | 57.6 | 2.4 | 1 | 39 | 56.6 | 2.4 | 2 | 39 | 55.6 | 2.4 | 3 |
| 38 | 59.6 | 2.4 | 0 | 38 | 58.6 | 2.4 | 1 | 38 | 57.6 | 2.4 | 2 | 38 | 56.6 | 2.4 | 3 |
| 37 | 60.6 | 2.4 | 0 | 37 | 59.6 | 2.4 | 1 | 37 | 58.6 | 2.4 | 2 | 37 | 57.6 | 2.4 | 3 |
| 36 | 61.6 | 2.4 | 0 | 36 | 60.6 | 2.4 | 1 | 36 | 59.6 | 2.4 | 2 | 36 | 58.6 | 2.4 | 3 |
| 35 | 62.6 | 2.4 | 0 | 35 | 61.6 | 2.4 | 1 | 35 | 60.6 | 2.4 | 2 | 35 | 59.6 | 2.4 | 3 |
| 34 | 63.6 | 2.4 | 0 | 34 | 62.6 | 2.4 | 1 | 34 | 61.6 | 2.4 | 2 | 34 | 60.6 | 2.4 | 3 |
| 33 | 64.6 | 2.4 | 0 | 33 | 63.6 | 2.4 | 1 | 33 | 62.6 | 2.4 | 2 | 33 | 61.6 | 2.4 | 3 |
| 32 | 65.6 | 2.4 | 0 | 32 | 64.6 | 2.4 | 1 | 32 | 63.6 | 2.4 | 2 | 32 | 62.6 | 2.4 | 3 |
| 31 | 66.6 | 2.4 | 0 | 31 | 65.6 | 2.4 | 1 | 31 | 64.6 | 2.4 | 2 | 31 | 63.6 | 2.4 | 3 |
| 30 | 67.6 | 2.4 | 0 | 30 | 66.6 | 2.4 | 1 | 30 | 65.6 | 2.4 | 2 | 30 | 64.6 | 2.4 | 3 |
| 60 | 37.4 | 2.6 | 0 | 60 | 36.4 | 2.6 | 1 | 60 | 35.4 | 2.6 | 2 | 60 | 34.4 | 2.6 | 3 |
| 59 | 38.4 | 2.6 | 0 | 59 | 37.4 | 2.6 | 1 | 59 | 36.4 | 2.6 | 2 | 59 | 35.4 | 2.6 | 3 |
| 58 | 39.4 | 2.6 | 0 | 58 | 38.4 | 2.6 | 1 | 58 | 37.4 | 2.6 | 2 | 58 | 36.4 | 2.6 | 3 |
| 57 | 40.4 | 2.6 | 0 | 57 | 39.4 | 2.6 | 1 | 57 | 38.4 | 2.6 | 2 | 57 | 37.4 | 2.6 | 3 |
| 56 | 41.4 | 2.6 | 0 | 56 | 40.4 | 2.6 | 1 | 56 | 39.4 | 2.6 | 2 | 56 | 38.4 | 2.6 | 3 |
| 55 | 42.4 | 2.6 | 0 | 55 | 41.4 | 2.6 | 1 | 55 | 40.4 | 2.6 | 2 | 55 | 39.4 | 2.6 | 3 |
| 54 | 43.4 | 2.6 | 0 | 54 | 42.4 | 2.6 | 1 | 54 | 41.4 | 2.6 | 2 | 54 | 40.4 | 2.6 | 3 |
| 53 | 44.4 | 2.6 | 0 | 53 | 43.4 | 2.6 | 1 | 53 | 42.4 | 2.6 | 2 | 53 | 41.4 | 2.6 | 3 |
| 52 | 45.4 | 2.6 | 0 | 52 | 44.4 | 2.6 | 1 | 52 | 43.4 | 2.6 | 2 | 52 | 42.4 | 2.6 | 3 |
| 51 | 46.4 | 2.6 | 0 | 51 | 45.4 | 2.6 | 1 | 51 | 44.4 | 2.6 | 2 | 51 | 43.4 | 2.6 | 3 |
| 50 | 47.4 | 2.6 | 0 | 50 | 46.4 | 2.6 | 1 | 50 | 45.4 | 2.6 | 2 | 50 | 44.4 | 2.6 | 3 |
| 49 | 48.4 | 2.6 | 0 | 49 | 47.4 | 2.6 | 1 | 49 | 46.4 | 2.6 | 2 | 49 | 45.4 | 2.6 | 3 |
| 48 | 49.4 | 2.6 | 0 | 48 | 48.4 | 2.6 | 1 | 48 | 47.4 | 2.6 | 2 | 48 | 46.4 | 2.6 | 3 |
| 47 | 50.4 | 2.6 | 0 | 47 | 49.4 | 2.6 | 1 | 47 | 48.4 | 2.6 | 2 | 47 | 47.4 | 2.6 | 3 |
| 46 | 51.4 | 2.6 | 0 | 46 | 50.4 | 2.6 | 1 | 46 | 49.4 | 2.6 | 2 | 46 | 48.4 | 2.6 | 3 |
| 45 | 52.4 | 2.6 | 0 | 45 | 51.4 | 2.6 | 1 | 45 | 50.4 | 2.6 | 2 | 45 | 49.4 | 2.6 | 3 |
| 44 | 53.4 | 2.6 | 0 | 44 | 52.4 | 2.6 | 1 | 44 | 51.4 | 2.6 | 2 | 44 | 50.4 | 2.6 | 3 |
| 43 | 54.4 | 2.6 | 0 | 43 | 53.4 | 2.6 | 1 | 43 | 52.4 | 2.6 | 2 | 43 | 51.4 | 2.6 | 3 |
| 42 | 55.4 | 2.6 | 0 | 42 | 54.4 | 2.6 | 1 | 42 | 53.4 | 2.6 | 2 | 42 | 52.4 | 2.6 | 3 |
| 41 | 56.4 | 2.6 | 0 | 41 | 55.4 | 2.6 | 1 | 41 | 54.4 | 2.6 | 2 | 41 | 53.4 | 2.6 | 3 |
| 40 | 57.4 | 2.6 | 0 | 40 | 56.4 | 2.6 | 1 | 40 | 55.4 | 2.6 | 2 | 40 | 54.4 | 2.6 | 3 |
| 39 | 58.4 | 2.6 | 0 | 39 | 57.4 | 2.6 | 1 | 39 | 56.4 | 2.6 | 2 | 39 | 55.4 | 2.6 | 3 |
| 38 | 59.4 | 2.6 | 0 | 38 | 58.4 | 2.6 | 1 | 38 | 57.4 | 2.6 | 2 | 38 | 56.4 | 2.6 | 3 |
| 37 | 60.4 | 2.6 | 0 | 37 | 59.4 | 2.6 | 1 | 37 | 58.4 | 2.6 | 2 | 37 | 57.4 | 2.6 | 3 |
| 36 | 61.4 | 2.6 | 0 | 36 | 60.4 | 2.6 | 1 | 36 | 59.4 | 2.6 | 2 | 36 | 58.4 | 2.6 | 3 |
| 35 | 62.4 | 2.6 | 0 | 35 | 61.4 | 2.6 | 1 | 35 | 60.4 | 2.6 | 2 | 35 | 59.4 | 2.6 | 3 |
| 34 | 63.4 | 2.6 | 0 | 34 | 62.4 | 2.6 | 1 | 34 | 61.4 | 2.6 | 2 | 34 | 60.4 | 2.6 | 3 |
| 33 | 64.4 | 2.6 | 0 | 33 | 63.4 | 2.6 | 1 | 33 | 62.4 | 2.6 | 2 | 33 | 61.4 | 2.6 | 3 |
| 32 | 65.4 | 2.6 | 0 | 32 | 64.4 | 2.6 | 1 | 32 | 63.4 | 2.6 | 2 | 32 | 62.4 | 2.6 | 3 |
| 31 | 66.4 | 2.6 | 0 | 31 | 65.4 | 2.6 | 1 | 31 | 64.4 | 2.6 | 2 | 31 | 63.4 | 2.6 | 3 |
| 30 | 67.4 | 2.6 | 0 | 30 | 66.4 | 2.6 | 1 | 30 | 65.4 | 2.6 | 2 | 30 | 64.4 | 2.6 | 3 |
| 60 | 37.2 | 2.8 | 0 | 60 | 36.2 | 2.8 | 1 | 60 | 35.2 | 2.8 | 2 | 60 | 34.2 | 2.8 | 3 |
| 59 | 38.2 | 2.8 | 0 | 59 | 37.2 | 2.8 | 1 | 59 | 36.2 | 2.8 | 2 | 59 | 35.2 | 2.8 | 3 |
| 58 | 39.2 | 2.8 | 0 | 58 | 38.2 | 2.8 | 1 | 58 | 37.2 | 2.8 | 2 | 58 | 36.2 | 2.8 | 3 |
| 57 | 40.2 | 2.8 | 0 | 57 | 39.2 | 2.8 | 1 | 57 | 38.2 | 2.8 | 2 | 57 | 37.2 | 2.8 | 3 |
| 56 | 41.2 | 2.8 | 0 | 56 | 40.2 | 2.8 | 1 | 56 | 39.2 | 2.8 | 2 | 56 | 38.2 | 2.8 | 3 |
| 55 | 42.2 | 2.8 | 0 | 55 | 41.2 | 2.8 | 1 | 55 | 40.2 | 2.8 | 2 | 55 | 39.2 | 2.8 | 3 |
| 54 | 43.2 | 2.8 | 0 | 54 | 42.2 | 2.8 | 1 | 54 | 41.2 | 2.8 | 2 | 54 | 40.2 | 2.8 | 3 |
| 53 | 44.2 | 2.8 | 0 | 53 | 43.2 | 2.8 | 1 | 53 | 42.2 | 2.8 | 2 | 53 | 41.2 | 2.8 | 3 |
| 52 | 45.2 | 2.8 | 0 | 52 | 44.2 | 2.8 | 1 | 52 | 43.2 | 2.8 | 2 | 52 | 42.2 | 2.8 | 3 |
| 51 | 46.2 | 2.8 | 0 | 51 | 45.2 | 2.8 | 1 | 51 | 44.2 | 2.8 | 2 | 51 | 43.2 | 2.8 | 3 |
| 50 | 47.2 | 2.8 | 0 | 50 | 46.2 | 2.8 | 1 | 50 | 45.2 | 2.8 | 2 | 50 | 44.2 | 2.8 | 3 |
| 49 | 48.2 | 2.8 | 0 | 49 | 47.2 | 2.8 | 1 | 49 | 46.2 | 2.8 | 2 | 49 | 45.2 | 2.8 | 3 |
| 48 | 49.2 | 2.8 | 0 | 48 | 48.2 | 2.8 | 1 | 48 | 47.2 | 2.8 | 2 | 48 | 46.2 | 2.8 | 3 |
| 47 | 50.2 | 2.8 | 0 | 47 | 49.2 | 2.8 | 1 | 47 | 48.2 | 2.8 | 2 | 47 | 47.2 | 2.8 | 3 |
| 46 | 51.2 | 2.8 | 0 | 46 | 50.2 | 2.8 | 1 | 46 | 49.2 | 2.8 | 2 | 46 | 48.2 | 2.8 | 3 |
| 45 | 52.2 | 2.8 | 0 | 45 | 51.2 | 2.8 | 1 | 45 | 50.2 | 2.8 | 2 | 45 | 49.2 | 2.8 | 3 |
| 44 | 53.2 | 2.8 | 0 | 44 | 52.2 | 2.8 | 1 | 44 | 51.2 | 2.8 | 2 | 44 | 50.2 | 2.8 | 3 |
| 43 | 54.2 | 2.8 | 0 | 43 | 53.2 | 2.8 | 1 | 43 | 52.2 | 2.8 | 2 | 43 | 51.2 | 2.8 | 3 |
| 42 | 55.2 | 2.8 | 0 | 42 | 54.2 | 2.8 | 1 | 42 | 53.2 | 2.8 | 2 | 42 | 52.2 | 2.8 | 3 |
| 41 | 56.2 | 2.8 | 0 | 41 | 55.2 | 2.8 | 1 | 41 | 54.2 | 2.8 | 2 | 41 | 53.2 | 2.8 | 3 |
| 40 | 57.2 | 2.8 | 0 | 40 | 56.2 | 2.8 | 1 | 40 | 55.2 | 2.8 | 2 | 40 | 54.2 | 2.8 | 3 |
| 39 | 58.2 | 2.8 | 0 | 39 | 57.2 | 2.8 | 1 | 39 | 56.2 | 2.8 | 2 | 39 | 55.2 | 2.8 | 3 |
| 38 | 59.2 | 2.8 | 0 | 38 | 58.2 | 2.8 | 1 | 38 | 57.2 | 2.8 | 2 | 38 | 56.2 | 2.8 | 3 |
| 37 | 60.2 | 2.8 | 0 | 37 | 59.2 | 2.8 | 1 | 37 | 58.2 | 2.8 | 2 | 37 | 57.2 | 2.8 | 3 |
| 36 | 61.2 | 2.8 | 0 | 36 | 60.2 | 2.8 | 1 | 36 | 59.2 | 2.8 | 2 | 36 | 58.2 | 2.8 | 3 |
| 35 | 62.2 | 2.8 | 0 | 35 | 61.2 | 2.8 | 1 | 35 | 60.2 | 2.8 | 2 | 35 | 59.2 | 2.8 | 3 |
| 34 | 63.2 | 2.8 | 0 | 34 | 62.2 | 2.8 | 1 | 34 | 61.2 | 2.8 | 2 | 34 | 60.2 | 2.8 | 3 |
| 33 | 64.2 | 2.8 | 0 | 33 | 63.2 | 2.8 | 1 | 33 | 62.2 | 2.8 | 2 | 33 | 61.2 | 2.8 | 3 |
| 32 | 65.2 | 2.8 | 0 | 32 | 64.2 | 2.8 | 1 | 32 | 63.2 | 2.8 | 2 | 32 | 62.2 | 2.8 | 3 |
| 31 | 66.2 | 2.8 | 0 | 31 | 65.2 | 2.8 | 1 | 31 | 64.2 | 2.8 | 2 | 31 | 63.2 | 2.8 | 3 |
| 30 | 67.2 | 2.8 | 0 | 30 | 66.2 | 2.8 | 1 | 30 | 65.2 | 2.8 | 2 | 30 | 64.2 | 2.8 | 3 |
| 60 | 37 | 3 | 0 | 60 | 36 | 3 | 1 | 60 | 35 | 3 | 2 | 60 | 34 | 3 | 3 |
| 59 | 38 | 3 | 0 | 59 | 37 | 3 | 1 | 59 | 36 | 3 | 2 | 59 | 35 | 3 | 3 |
| 58 | 39 | 3 | 0 | 58 | 38 | 3 | 1 | 58 | 37 | 3 | 2 | 58 | 36 | 3 | 3 |
| 57 | 40 | 3 | 0 | 57 | 39 | 3 | 1 | 57 | 38 | 3 | 2 | 57 | 37 | 3 | 3 |
| 56 | 41 | 3 | 0 | 56 | 40 | 3 | 1 | 56 | 39 | 3 | 2 | 56 | 38 | 3 | 3 |
| 55 | 42 | 3 | 0 | 55 | 41 | 3 | 1 | 55 | 40 | 3 | 2 | 55 | 39 | 3 | 3 |
| 54 | 43 | 3 | 0 | 54 | 42 | 3 | 1 | 54 | 41 | 3 | 2 | 54 | 40 | 3 | 3 |
| 53 | 44 | 3 | 0 | 53 | 43 | 3 | 1 | 53 | 42 | 3 | 2 | 53 | 41 | 3 | 3 |
| 52 | 45 | 3 | 0 | 52 | 44 | 3 | 1 | 52 | 43 | 3 | 2 | 52 | 42 | 3 | 3 |
| 51 | 46 | 3 | 0 | 51 | 45 | 3 | 1 | 51 | 44 | 3 | 2 | 51 | 43 | 3 | 3 |
| 50 | 47 | 3 | 0 | 50 | 46 | 3 | 1 | 50 | 45 | 3 | 2 | 50 | 44 | 3 | 3 |
| 49 | 48 | 3 | 0 | 49 | 47 | 3 | 1 | 49 | 46 | 3 | 2 | 49 | 45 | 3 | 3 |
| 48 | 49 | 3 | 0 | 48 | 48 | 3 | 1 | 48 | 47 | 3 | 2 | 48 | 46 | 3 | 3 |
| 47 | 50 | 3 | 0 | 47 | 49 | 3 | 1 | 47 | 48 | 3 | 2 | 47 | 47 | 3 | 3 |
| 46 | 51 | 3 | 0 | 46 | 50 | 3 | 1 | 46 | 49 | 3 | 2 | 46 | 48 | 3 | 3 |
| 45 | 52 | 3 | 0 | 45 | 51 | 3 | 1 | 45 | 50 | 3 | 2 | 45 | 49 | 3 | 3 |
| 44 | 53 | 3 | 0 | 44 | 52 | 3 | 1 | 44 | 51 | 3 | 2 | 44 | 50 | 3 | 3 |
| 43 | 54 | 3 | 0 | 43 | 53 | 3 | 1 | 43 | 52 | 3 | 2 | 43 | 51 | 3 | 3 |
| 42 | 55 | 3 | 0 | 42 | 54 | 3 | 1 | 42 | 53 | 3 | 2 | 42 | 52 | 3 | 3 |
| 41 | 56 | 3 | 0 | 41 | 55 | 3 | 1 | 41 | 54 | 3 | 2 | 41 | 53 | 3 | 3 |
| 40 | 57 | 3 | 0 | 40 | 56 | 3 | 1 | 40 | 55 | 3 | 2 | 40 | 54 | 3 | 3 |
| 39 | 58 | 3 | 0 | 39 | 57 | 3 | 1 | 39 | 56 | 3 | 2 | 39 | 55 | 3 | 3 |
| 38 | 59 | 3 | 0 | 38 | 58 | 3 | 1 | 38 | 57 | 3 | 2 | 38 | 56 | 3 | 3 |
| 37 | 60 | 3 | 0 | 37 | 59 | 3 | 1 | 37 | 58 | 3 | 2 | 37 | 57 | 3 | 3 |
| 36 | 61 | 3 | 0 | 36 | 60 | 3 | 1 | 36 | 59 | 3 | 2 | 36 | 58 | 3 | 3 |
| 35 | 62 | 3 | 0 | 35 | 61 | 3 | 1 | 35 | 60 | 3 | 2 | 35 | 59 | 3 | 3 |
| 34 | 63 | 3 | 0 | 34 | 62 | 3 | 1 | 34 | 61 | 3 | 2 | 34 | 60 | 3 | 3 |
| 33 | 64 | 3 | 0 | 33 | 63 | 3 | 1 | 33 | 62 | 3 | 2 | 33 | 61 | 3 | 3 |
| 32 | 65 | 3 | 0 | 32 | 64 | 3 | 1 | 32 | 63 | 3 | 2 | 32 | 62 | 3 | 3 |
| 31 | 66 | 3 | 0 | 31 | 65 | 3 | 1 | 31 | 64 | 3 | 2 | 31 | 63 | 3 | 3 |
| 30 | 67 | 3 | 0 | 30 | 66 | 3 | 1 | 30 | 65 | 3 | 2 | 30 | 64 | 3 | 3 |
| 60 | 35 | 1 | 4 | 60 | 34 | 1 | 5 | 60 | 33 | 1 | 6 | 60 | 32 | 1 | 7 |
| 59 | 36 | 1 | 4 | 59 | 35 | 1 | 5 | 59 | 34 | 1 | 6 | 59 | 33 | 1 | 7 |
| 58 | 37 | 1 | 4 | 58 | 36 | 1 | 5 | 58 | 35 | 1 | 6 | 58 | 34 | 1 | 7 |
| 57 | 38 | 1 | 4 | 57 | 37 | 1 | 5 | 57 | 36 | 1 | 6 | 57 | 35 | 1 | 7 |
| 56 | 39 | 1 | 4 | 56 | 38 | 1 | 5 | 56 | 37 | 1 | 6 | 56 | 36 | 1 | 7 |
| 55 | 40 | 1 | 4 | 55 | 39 | 1 | 5 | 55 | 38 | 1 | 6 | 55 | 37 | 1 | 7 |
| 54 | 41 | 1 | 4 | 54 | 40 | 1 | 5 | 54 | 39 | 1 | 6 | 54 | 38 | 1 | 7 |
| 53 | 42 | 1 | 4 | 53 | 41 | 1 | 5 | 53 | 40 | 1 | 6 | 53 | 39 | 1 | 7 |
| 52 | 43 | 1 | 4 | 52 | 42 | 1 | 5 | 52 | 41 | 1 | 6 | 52 | 40 | 1 | 7 |
| 51 | 44 | 1 | 4 | 51 | 43 | 1 | 5 | 51 | 42 | 1 | 6 | 51 | 41 | 1 | 7 |
| 50 | 45 | 1 | 4 | 50 | 44 | 1 | 5 | 50 | 43 | 1 | 6 | 50 | 42 | 1 | 7 |
| 49 | 46 | 1 | 4 | 49 | 45 | 1 | 5 | 49 | 44 | 1 | 6 | 49 | 43 | 1 | 7 |
| 48 | 47 | 1 | 4 | 48 | 46 | 1 | 5 | 48 | 45 | 1 | 6 | 48 | 44 | 1 | 7 |
| 47 | 48 | 1 | 4 | 47 | 47 | 1 | 5 | 47 | 46 | 1 | 6 | 47 | 45 | 1 | 7 |
| 46 | 49 | 1 | 4 | 46 | 48 | 1 | 5 | 46 | 47 | 1 | 6 | 46 | 46 | 1 | 7 |
| 45 | 50 | 1 | 4 | 45 | 49 | 1 | 5 | 45 | 48 | 1 | 6 | 45 | 47 | 1 | 7 |
| 44 | 51 | 1 | 4 | 44 | 50 | 1 | 5 | 44 | 49 | 1 | 6 | 44 | 48 | 1 | 7 |
| 43 | 52 | 1 | 4 | 43 | 51 | 1 | 5 | 43 | 50 | 1 | 6 | 43 | 49 | 1 | 7 |
| 42 | 53 | 1 | 4 | 42 | 52 | 1 | 5 | 42 | 51 | 1 | 6 | 42 | 50 | 1 | 7 |
| 41 | 54 | 1 | 4 | 41 | 53 | 1 | 5 | 41 | 52 | 1 | 6 | 41 | 51 | 1 | 7 |
| 40 | 55 | 1 | 4 | 40 | 54 | 1 | 5 | 40 | 53 | 1 | 6 | 40 | 52 | 1 | 7 |
| 39 | 56 | 1 | 4 | 39 | 55 | 1 | 5 | 39 | 54 | 1 | 6 | 39 | 53 | 1 | 7 |
| 38 | 57 | 1 | 4 | 38 | 56 | 1 | 5 | 38 | 55 | 1 | 6 | 38 | 54 | 1 | 7 |
| 37 | 58 | 1 | 4 | 37 | 57 | 1 | 5 | 37 | 56 | 1 | 6 | 37 | 55 | 1 | 7 |
| 36 | 59 | 1 | 4 | 36 | 58 | 1 | 5 | 36 | 57 | 1 | 6 | 36 | 56 | 1 | 7 |
| 35 | 60 | 1 | 4 | 35 | 59 | 1 | 5 | 35 | 58 | 1 | 6 | 35 | 57 | 1 | 7 |
| 34 | 61 | 1 | 4 | 34 | 60 | 1 | 5 | 34 | 59 | 1 | 6 | 34 | 58 | 1 | 7 |
| 33 | 62 | 1 | 4 | 33 | 61 | 1 | 5 | 33 | 60 | 1 | 6 | 33 | 59 | 1 | 7 |
| 32 | 63 | 1 | 4 | 32 | 62 | 1 | 5 | 32 | 61 | 1 | 6 | 32 | 60 | 1 | 7 |
| 31 | 64 | 1 | 4 | 31 | 63 | 1 | 5 | 31 | 62 | 1 | 6 | 31 | 61 | 1 | 7 |
| 30 | 65 | 1 | 4 | 30 | 64 | 1 | 5 | 30 | 63 | 1 | 6 | 30 | 62 | 1 | 7 |
| 60 | 34.8 | 1.2 | 4 | 60 | 33.8 | 1.2 | 5 | 60 | 32.8 | 1.2 | 6 | 60 | 31.8 | 1.2 | 7 |
| 59 | 35.8 | 1.2 | 4 | 59 | 34.8 | 1.2 | 5 | 59 | 33.8 | 1.2 | 6 | 59 | 32.8 | 1.2 | 7 |
| 58 | 36.8 | 1.2 | 4 | 58 | 35.8 | 1.2 | 5 | 58 | 34.8 | 1.2 | 6 | 58 | 33.8 | 1.2 | 7 |
| 57 | 37.8 | 1.2 | 4 | 57 | 36.8 | 1.2 | 5 | 57 | 35.8 | 1.2 | 6 | 57 | 34.8 | 1.2 | 7 |
| 56 | 38.8 | 1.2 | 4 | 56 | 37.8 | 1.2 | 5 | 56 | 36.8 | 1.2 | 6 | 56 | 35.8 | 1.2 | 7 |
| 55 | 39.8 | 1.2 | 4 | 55 | 38.8 | 1.2 | 5 | 55 | 37.8 | 1.2 | 6 | 55 | 36.8 | 1.2 | 7 |
| 54 | 40.8 | 1.2 | 4 | 54 | 39.8 | 1.2 | 5 | 54 | 38.8 | 1.2 | 6 | 54 | 37.8 | 1.2 | 7 |
| 53 | 41.8 | 1.2 | 4 | 53 | 40.8 | 1.2 | 5 | 53 | 39.8 | 1.2 | 6 | 53 | 38.8 | 1.2 | 7 |
| 52 | 42.8 | 1.2 | 4 | 52 | 41.8 | 1.2 | 5 | 52 | 40.8 | 1.2 | 6 | 52 | 39.8 | 1.2 | 7 |
| 51 | 43.8 | 1.2 | 4 | 51 | 42.8 | 1.2 | 5 | 51 | 41.8 | 1.2 | 6 | 51 | 40.8 | 1.2 | 7 |
| 50 | 44.8 | 1.2 | 4 | 50 | 43.8 | 1.2 | 5 | 50 | 42.8 | 1.2 | 6 | 50 | 41.8 | 1.2 | 7 |
| 49 | 45.8 | 1.2 | 4 | 49 | 44.8 | 1.2 | 5 | 49 | 43.8 | 1.2 | 6 | 49 | 42.8 | 1.2 | 7 |
| 48 | 46.8 | 1.2 | 4 | 48 | 45.8 | 1.2 | 5 | 48 | 44.8 | 1.2 | 6 | 48 | 43.8 | 1.2 | 7 |
| 47 | 47.8 | 1.2 | 4 | 47 | 46.8 | 1.2 | 5 | 47 | 45.8 | 1.2 | 6 | 47 | 44.8 | 1.2 | 7 |
| 46 | 48.8 | 1.2 | 4 | 46 | 47.8 | 1.2 | 5 | 46 | 46.8 | 1.2 | 6 | 46 | 45.8 | 1.2 | 7 |
| 45 | 49.8 | 1.2 | 4 | 45 | 48.8 | 1.2 | 5 | 45 | 47.8 | 1.2 | 6 | 45 | 46.8 | 1.2 | 7 |
| 44 | 50.8 | 1.2 | 4 | 44 | 49.8 | 1.2 | 5 | 44 | 48.8 | 1.2 | 6 | 44 | 47.8 | 1.2 | 7 |
| 43 | 51.8 | 1.2 | 4 | 43 | 50.8 | 1.2 | 5 | 43 | 49.8 | 1.2 | 6 | 43 | 48.8 | 1.2 | 7 |
| 42 | 52.8 | 1.2 | 4 | 42 | 51.8 | 1.2 | 5 | 42 | 50.8 | 1.2 | 6 | 42 | 49.8 | 1.2 | 7 |
| 41 | 53.8 | 1.2 | 4 | 41 | 52.8 | 1.2 | 5 | 41 | 51.8 | 1.2 | 6 | 41 | 50.8 | 1.2 | 7 |
| 40 | 54.8 | 1.2 | 4 | 40 | 53.8 | 1.2 | 5 | 40 | 52.8 | 1.2 | 6 | 40 | 51.8 | 1.2 | 7 |
| 39 | 55.8 | 1.2 | 4 | 39 | 54.8 | 1.2 | 5 | 39 | 53.8 | 1.2 | 6 | 39 | 52.8 | 1.2 | 7 |
| 38 | 56.8 | 1.2 | 4 | 38 | 55.8 | 1.2 | 5 | 38 | 54.8 | 1.2 | 6 | 38 | 53.8 | 1.2 | 7 |
| 37 | 57.8 | 1.2 | 4 | 37 | 56.8 | 1.2 | 5 | 37 | 55.8 | 1.2 | 6 | 37 | 54.8 | 1.2 | 7 |
| 36 | 58.8 | 1.2 | 4 | 36 | 57.8 | 1.2 | 5 | 36 | 56.8 | 1.2 | 6 | 36 | 55.8 | 1.2 | 7 |
| 35 | 59.8 | 1.2 | 4 | 35 | 58.8 | 1.2 | 5 | 35 | 57.8 | 1.2 | 6 | 35 | 56.8 | 1.2 | 7 |
| 34 | 60.8 | 1.2 | 4 | 34 | 59.8 | 1.2 | 5 | 34 | 58.8 | 1.2 | 6 | 34 | 57.8 | 1.2 | 7 |
| 33 | 61.8 | 1.2 | 4 | 33 | 60.8 | 1.2 | 5 | 33 | 59.8 | 1.2 | 6 | 33 | 58.8 | 1.2 | 7 |
| 32 | 62.8 | 1.2 | 4 | 32 | 61.8 | 1.2 | 5 | 32 | 60.8 | 1.2 | 6 | 32 | 59.8 | 1.2 | 7 |
| 31 | 63.8 | 1.2 | 4 | 31 | 62.8 | 1.2 | 5 | 31 | 61.8 | 1.2 | 6 | 31 | 60.8 | 1.2 | 7 |
| 30 | 64.8 | 1.2 | 4 | 30 | 63.8 | 1.2 | 5 | 30 | 62.8 | 1.2 | 6 | 30 | 61.8 | 1.2 | 7 |
| 60 | 34.6 | 1.4 | 4 | 60 | 33.6 | 1.4 | 5 | 60 | 32.6 | 1.4 | 6 | 60 | 31.6 | 1.4 | 7 |
| 59 | 35.6 | 1.4 | 4 | 59 | 34.6 | 1.4 | 5 | 59 | 33.6 | 1.4 | 6 | 59 | 32.6 | 1.4 | 7 |
| 58 | 36.6 | 1.4 | 4 | 58 | 35.6 | 1.4 | 5 | 58 | 34.6 | 1.4 | 6 | 58 | 33.6 | 1.4 | 7 |
| 57 | 37.6 | 1.4 | 4 | 57 | 36.6 | 1.4 | 5 | 57 | 35.6 | 1.4 | 6 | 57 | 34.6 | 1.4 | 7 |
| 56 | 38.6 | 1.4 | 4 | 56 | 37.6 | 1.4 | 5 | 56 | 36.6 | 1.4 | 6 | 56 | 35.6 | 1.4 | 7 |
| 55 | 39.6 | 1.4 | 4 | 55 | 38.6 | 1.4 | 5 | 55 | 37.6 | 1.4 | 6 | 55 | 36.6 | 1.4 | 7 |
| 54 | 40.6 | 1.4 | 4 | 54 | 39.6 | 1.4 | 5 | 54 | 38.6 | 1.4 | 6 | 54 | 37.6 | 1.4 | 7 |
| 53 | 41.6 | 1.4 | 4 | 53 | 40.6 | 1.4 | 5 | 53 | 39.6 | 1.4 | 6 | 53 | 38.6 | 1.4 | 7 |
| 52 | 42.6 | 1.4 | 4 | 52 | 41.6 | 1.4 | 5 | 52 | 40.6 | 1.4 | 6 | 52 | 39.6 | 1.4 | 7 |
| 51 | 43.6 | 1.4 | 4 | 51 | 42.6 | 1.4 | 5 | 51 | 41.6 | 1.4 | 6 | 51 | 40.6 | 1.4 | 7 |
| 50 | 44.6 | 1.4 | 4 | 50 | 43.6 | 1.4 | 5 | 50 | 42.6 | 1.4 | 6 | 50 | 41.6 | 1.4 | 7 |
| 49 | 45.6 | 1.4 | 4 | 49 | 44.6 | 1.4 | 5 | 49 | 43.6 | 1.4 | 6 | 49 | 42.6 | 1.4 | 7 |
| 48 | 46.6 | 1.4 | 4 | 48 | 45.6 | 1.4 | 5 | 48 | 44.6 | 1.4 | 6 | 48 | 43.6 | 1.4 | 7 |
| 47 | 47.6 | 1.4 | 4 | 47 | 46.6 | 1.4 | 5 | 47 | 45.6 | 1.4 | 6 | 47 | 44.6 | 1.4 | 7 |
| 46 | 48.6 | 1.4 | 4 | 46 | 47.6 | 1.4 | 5 | 46 | 46.6 | 1.4 | 6 | 46 | 45.6 | 1.4 | 7 |
| 45 | 49.6 | 1.4 | 4 | 45 | 48.6 | 1.4 | 5 | 45 | 47.6 | 1.4 | 6 | 45 | 46.6 | 1.4 | 7 |
| 44 | 50.6 | 1.4 | 4 | 44 | 49.6 | 1.4 | 5 | 44 | 48.6 | 1.4 | 6 | 44 | 47.6 | 1.4 | 7 |
| 43 | 51.6 | 1.4 | 4 | 43 | 50.6 | 1.4 | 5 | 43 | 49.6 | 1.4 | 6 | 43 | 48.6 | 1.4 | 7 |
| 42 | 52.6 | 1.4 | 4 | 42 | 51.6 | 1.4 | 5 | 42 | 50.6 | 1.4 | 6 | 42 | 49.6 | 1.4 | 7 |
| 41 | 53.6 | 1.4 | 4 | 41 | 52.6 | 1.4 | 5 | 41 | 51.6 | 1.4 | 6 | 41 | 50.6 | 1.4 | 7 |
| 40 | 54.6 | 1.4 | 4 | 40 | 53.6 | 1.4 | 5 | 40 | 52.6 | 1.4 | 6 | 40 | 51.6 | 1.4 | 7 |
| 39 | 55.6 | 1.4 | 4 | 39 | 54.6 | 1.4 | 5 | 39 | 53.6 | 1.4 | 6 | 39 | 52.6 | 1.4 | 7 |
| 38 | 56.6 | 1.4 | 4 | 38 | 55.6 | 1.4 | 5 | 38 | 54.6 | 1.4 | 6 | 38 | 53.6 | 1.4 | 7 |
| 37 | 57.6 | 1.4 | 4 | 37 | 56.6 | 1.4 | 5 | 37 | 55.6 | 1.4 | 6 | 37 | 54.6 | 1.4 | 7 |
| 36 | 58.6 | 1.4 | 4 | 36 | 57.6 | 1.4 | 5 | 36 | 56.6 | 1.4 | 6 | 36 | 55.6 | 1.4 | 7 |
| 35 | 59.6 | 1.4 | 4 | 35 | 58.6 | 1.4 | 5 | 35 | 57.6 | 1.4 | 6 | 35 | 56.6 | 1.4 | 7 |
| 34 | 60.6 | 1.4 | 4 | 34 | 59.6 | 1.4 | 5 | 34 | 58.6 | 1.4 | 6 | 34 | 57.6 | 1.4 | 7 |
| 33 | 61.6 | 1.4 | 4 | 33 | 60.6 | 1.4 | 5 | 33 | 59.6 | 1.4 | 6 | 33 | 58.6 | 1.4 | 7 |
| 32 | 62.6 | 1.4 | 4 | 32 | 61.6 | 1.4 | 5 | 32 | 60.6 | 1.4 | 6 | 32 | 59.6 | 1.4 | 7 |
| 31 | 63.6 | 1.4 | 4 | 31 | 62.6 | 1.4 | 5 | 31 | 61.6 | 1.4 | 6 | 31 | 60.6 | 1.4 | 7 |
| 30 | 64.6 | 1.4 | 4 | 30 | 63.6 | 1.4 | 5 | 30 | 62.6 | 1.4 | 6 | 30 | 61.6 | 1.4 | 7 |
| 60 | 34.4 | 1.6 | 4 | 60 | 33.4 | 1.6 | 5 | 60 | 32.4 | 1.6 | 6 | 60 | 31.4 | 1.6 | 7 |
| 59 | 35.4 | 1.6 | 4 | 59 | 34.4 | 1.6 | 5 | 59 | 33.4 | 1.6 | 6 | 59 | 32.4 | 1.6 | 7 |
| 58 | 36.4 | 1.6 | 4 | 58 | 35.4 | 1.6 | 5 | 58 | 34.4 | 1.6 | 6 | 58 | 33.4 | 1.6 | 7 |
| 57 | 37.4 | 1.6 | 4 | 57 | 36.4 | 1.6 | 5 | 57 | 35.4 | 1.6 | 6 | 57 | 34.4 | 1.6 | 7 |
| 56 | 38.4 | 1.6 | 4 | 56 | 37.4 | 1.6 | 5 | 56 | 36.4 | 1.6 | 6 | 56 | 35.4 | 1.6 | 7 |
| 55 | 39.4 | 1.6 | 4 | 55 | 38.4 | 1.6 | 5 | 55 | 37.4 | 1.6 | 6 | 55 | 36.4 | 1.6 | 7 |
| 54 | 40.4 | 1.6 | 4 | 54 | 39.4 | 1.6 | 5 | 54 | 38.4 | 1.6 | 6 | 54 | 37.4 | 1.6 | 7 |
| 53 | 41.4 | 1.6 | 4 | 53 | 40.4 | 1.6 | 5 | 53 | 39.4 | 1.6 | 6 | 53 | 38.4 | 1.6 | 7 |
| 52 | 42.4 | 1.6 | 4 | 52 | 41.4 | 1.6 | 5 | 52 | 40.4 | 1.6 | 6 | 52 | 39.4 | 1.6 | 7 |
| 51 | 43.4 | 1.6 | 4 | 51 | 42.4 | 1.6 | 5 | 51 | 41.4 | 1.6 | 6 | 51 | 40.4 | 1.6 | 7 |
| 50 | 44.4 | 1.6 | 4 | 50 | 43.4 | 1.6 | 5 | 50 | 42.4 | 1.6 | 6 | 50 | 41.4 | 1.6 | 7 |
| 49 | 45.4 | 1.6 | 4 | 49 | 44.4 | 1.6 | 5 | 49 | 43.4 | 1.6 | 6 | 49 | 42.4 | 1.6 | 7 |
| 48 | 46.4 | 1.6 | 4 | 48 | 45.4 | 1.6 | 5 | 48 | 44.4 | 1.6 | 6 | 48 | 43.4 | 1.6 | 7 |
| 47 | 47.4 | 1.6 | 4 | 47 | 46.4 | 1.6 | 5 | 47 | 45.4 | 1.6 | 6 | 47 | 44.4 | 1.6 | 7 |
| 46 | 48.4 | 1.6 | 4 | 46 | 47.4 | 1.6 | 5 | 46 | 46.4 | 1.6 | 6 | 46 | 45.4 | 1.6 | 7 |
| 45 | 49.4 | 1.6 | 4 | 45 | 48.4 | 1.6 | 5 | 45 | 47.4 | 1.6 | 6 | 45 | 46.4 | 1.6 | 7 |
| 44 | 50.4 | 1.6 | 4 | 44 | 49.4 | 1.6 | 5 | 44 | 48.4 | 1.6 | 6 | 44 | 47.4 | 1.6 | 7 |
| 43 | 51.4 | 1.6 | 4 | 43 | 50.4 | 1.6 | 5 | 43 | 49.4 | 1.6 | 6 | 43 | 48.4 | 1.6 | 7 |
| 42 | 52.4 | 1.6 | 4 | 42 | 51.4 | 1.6 | 5 | 42 | 50.4 | 1.6 | 6 | 42 | 49.4 | 1.6 | 7 |
| 41 | 53.4 | 1.6 | 4 | 41 | 52.4 | 1.6 | 5 | 41 | 51.4 | 1.6 | 6 | 41 | 50.4 | 1.6 | 7 |
| 40 | 54.4 | 1.6 | 4 | 40 | 53.4 | 1.6 | 5 | 40 | 52.4 | 1.6 | 6 | 40 | 51.4 | 1.6 | 7 |
| 39 | 55.4 | 1.6 | 4 | 39 | 54.4 | 1.6 | 5 | 39 | 53.4 | 1.6 | 6 | 39 | 52.4 | 1.6 | 7 |
| 38 | 56.4 | 1.6 | 4 | 38 | 55.4 | 1.6 | 5 | 38 | 54.4 | 1.6 | 6 | 38 | 53.4 | 1.6 | 7 |
| 37 | 57.4 | 1.6 | 4 | 37 | 56.4 | 1.6 | 5 | 37 | 55.4 | 1.6 | 6 | 37 | 54.4 | 1.6 | 7 |
| 36 | 58.4 | 1.6 | 4 | 36 | 57.4 | 1.6 | 5 | 36 | 56.4 | 1.6 | 6 | 36 | 55.4 | 1.6 | 7 |
| 35 | 59.4 | 1.6 | 4 | 35 | 58.4 | 1.6 | 5 | 35 | 57.4 | 1.6 | 6 | 35 | 56.4 | 1.6 | 7 |
| 34 | 60.4 | 1.6 | 4 | 34 | 59.4 | 1.6 | 5 | 34 | 58.4 | 1.6 | 6 | 34 | 57.4 | 1.6 | 7 |
| 33 | 61.4 | 1.6 | 4 | 33 | 60.4 | 1.6 | 5 | 33 | 59.4 | 1.6 | 6 | 33 | 58.4 | 1.6 | 7 |
| 32 | 62.4 | 1.6 | 4 | 32 | 61.4 | 1.6 | 5 | 32 | 60.4 | 1.6 | 6 | 32 | 59.4 | 1.6 | 7 |
| 31 | 63.4 | 1.6 | 4 | 31 | 62.4 | 1.6 | 5 | 31 | 61.4 | 1.6 | 6 | 31 | 60.4 | 1.6 | 7 |
| 30 | 64.4 | 1.6 | 4 | 30 | 63.4 | 1.6 | 5 | 30 | 62.4 | 1.6 | 6 | 30 | 61.4 | 1.6 | 7 |
| 60 | 34.2 | 1.8 | 4 | 60 | 33.2 | 1.8 | 5 | 60 | 32.2 | 1.8 | 6 | 60 | 31.2 | 1.8 | 7 |
| 59 | 35.2 | 1.8 | 4 | 59 | 34.2 | 1.8 | 5 | 59 | 33.2 | 1.8 | 6 | 59 | 32.2 | 1.8 | 7 |
| 58 | 36.2 | 1.8 | 4 | 58 | 35.2 | 1.8 | 5 | 58 | 34.2 | 1.8 | 6 | 58 | 33.2 | 1.8 | 7 |
| 57 | 37.2 | 1.8 | 4 | 57 | 36.2 | 1.8 | 5 | 57 | 35.2 | 1.8 | 6 | 57 | 34.2 | 1.8 | 7 |
| 56 | 38.2 | 1.8 | 4 | 56 | 37.2 | 1.8 | 5 | 56 | 36.2 | 1.8 | 6 | 56 | 35.2 | 1.8 | 7 |
| 55 | 39.2 | 1.8 | 4 | 55 | 38.2 | 1.8 | 5 | 55 | 37.2 | 1.8 | 6 | 55 | 36.2 | 1.8 | 7 |
| 54 | 40.2 | 1.8 | 4 | 54 | 39.2 | 1.8 | 5 | 54 | 38.2 | 1.8 | 6 | 54 | 37.2 | 1.8 | 7 |
| 53 | 41.2 | 1.8 | 4 | 53 | 40.2 | 1.8 | 5 | 53 | 39.2 | 1.8 | 6 | 53 | 38.2 | 1.8 | 7 |
| 52 | 42.2 | 1.8 | 4 | 52 | 41.2 | 1.8 | 5 | 52 | 40.2 | 1.8 | 6 | 52 | 39.2 | 1.8 | 7 |
| 51 | 43.2 | 1.8 | 4 | 51 | 42.2 | 1.8 | 5 | 51 | 41.2 | 1.8 | 6 | 51 | 40.2 | 1.8 | 7 |
| 50 | 44.2 | 1.8 | 4 | 50 | 43.2 | 1.8 | 5 | 50 | 42.2 | 1.8 | 6 | 50 | 41.2 | 1.8 | 7 |
| 49 | 45.2 | 1.8 | 4 | 49 | 44.2 | 1.8 | 5 | 49 | 43.2 | 1.8 | 6 | 49 | 42.2 | 1.8 | 7 |
| 48 | 46.2 | 1.8 | 4 | 48 | 45.2 | 1.8 | 5 | 48 | 44.2 | 1.8 | 6 | 48 | 43.2 | 1.8 | 7 |
| 47 | 47.2 | 1.8 | 4 | 47 | 46.2 | 1.8 | 5 | 47 | 45.2 | 1.8 | 6 | 47 | 44.2 | 1.8 | 7 |
| 46 | 48.2 | 1.8 | 4 | 46 | 47.2 | 1.8 | 5 | 46 | 46.2 | 1.8 | 6 | 46 | 45.2 | 1.8 | 7 |
| 45 | 49.2 | 1.8 | 4 | 45 | 48.2 | 1.8 | 5 | 45 | 47.2 | 1.8 | 6 | 45 | 46.2 | 1.8 | 7 |
| 44 | 50.2 | 1.8 | 4 | 44 | 49.2 | 1.8 | 5 | 44 | 48.2 | 1.8 | 6 | 44 | 47.2 | 1.8 | 7 |
| 43 | 51.2 | 1.8 | 4 | 43 | 50.2 | 1.8 | 5 | 43 | 49.2 | 1.8 | 6 | 43 | 48.2 | 1.8 | 7 |
| 42 | 52.2 | 1.8 | 4 | 42 | 51.2 | 1.8 | 5 | 42 | 50.2 | 1.8 | 6 | 42 | 49.2 | 1.8 | 7 |
| 41 | 53.2 | 1.8 | 4 | 41 | 52.2 | 1.8 | 5 | 41 | 51.2 | 1.8 | 6 | 41 | 50.2 | 1.8 | 7 |
| 40 | 54.2 | 1.8 | 4 | 40 | 53.2 | 1.8 | 5 | 40 | 52.2 | 1.8 | 6 | 40 | 51.2 | 1.8 | 7 |
| 39 | 55.2 | 1.8 | 4 | 39 | 54.2 | 1.8 | 5 | 39 | 53.2 | 1.8 | 6 | 39 | 52.2 | 1.8 | 7 |
| 38 | 56.2 | 1.8 | 4 | 38 | 55.2 | 1.8 | 5 | 38 | 54.2 | 1.8 | 6 | 38 | 53.2 | 1.8 | 7 |
| 37 | 57.2 | 1.8 | 4 | 37 | 56.2 | 1.8 | 5 | 37 | 55.2 | 1.8 | 6 | 37 | 54.2 | 1.8 | 7 |
| 36 | 58.2 | 1.8 | 4 | 36 | 57.2 | 1.8 | 5 | 36 | 56.2 | 1.8 | 6 | 36 | 55.2 | 1.8 | 7 |
| 35 | 59.2 | 1.8 | 4 | 35 | 58.2 | 1.8 | 5 | 35 | 57.2 | 1.8 | 6 | 35 | 56.2 | 1.8 | 7 |
| 34 | 60.2 | 1.8 | 4 | 34 | 59.2 | 1.8 | 5 | 34 | 58.2 | 1.8 | 6 | 34 | 57.2 | 1.8 | 7 |
| 33 | 61.2 | 1.8 | 4 | 33 | 60.2 | 1.8 | 5 | 33 | 59.2 | 1.8 | 6 | 33 | 58.2 | 1.8 | 7 |
| 32 | 62.2 | 1.8 | 4 | 32 | 61.2 | 1.8 | 5 | 32 | 60.2 | 1.8 | 6 | 32 | 59.2 | 1.8 | 7 |
| 31 | 63.2 | 1.8 | 4 | 31 | 62.2 | 1.8 | 5 | 31 | 61.2 | 1.8 | 6 | 31 | 60.2 | 1.8 | 7 |
| 30 | 64.2 | 1.8 | 4 | 30 | 63.2 | 1.8 | 5 | 30 | 62.2 | 1.8 | 6 | 30 | 61.2 | 1.8 | 7 |
| 60 | 34 | 2 | 4 | 60 | 33 | 2 | 5 | 60 | 32 | 2 | 6 | 60 | 31 | 2 | 7 |
| 59 | 35 | 2 | 4 | 59 | 34 | 2 | 5 | 59 | 33 | 2 | 6 | 59 | 32 | 2 | 7 |
| 58 | 36 | 2 | 4 | 58 | 35 | 2 | 5 | 58 | 34 | 2 | 6 | 58 | 33 | 2 | 7 |
| 57 | 37 | 2 | 4 | 57 | 36 | 2 | 5 | 57 | 35 | 2 | 6 | 57 | 34 | 2 | 7 |
| 56 | 38 | 2 | 4 | 56 | 37 | 2 | 5 | 56 | 36 | 2 | 6 | 56 | 35 | 2 | 7 |
| 55 | 39 | 2 | 4 | 55 | 38 | 2 | 5 | 55 | 37 | 2 | 6 | 55 | 36 | 2 | 7 |
| 54 | 40 | 2 | 4 | 54 | 39 | 2 | 5 | 54 | 38 | 2 | 6 | 54 | 37 | 2 | 7 |
| 53 | 41 | 2 | 4 | 53 | 40 | 2 | 5 | 53 | 39 | 2 | 6 | 53 | 38 | 2 | 7 |
| 52 | 42 | 2 | 4 | 52 | 41 | 2 | 5 | 52 | 40 | 2 | 6 | 52 | 39 | 2 | 7 |
| 51 | 43 | 2 | 4 | 51 | 42 | 2 | 5 | 51 | 41 | 2 | 6 | 51 | 40 | 2 | 7 |
| 50 | 44 | 2 | 4 | 50 | 43 | 2 | 5 | 50 | 42 | 2 | 6 | 50 | 41 | 2 | 7 |
| 49 | 45 | 2 | 4 | 49 | 44 | 2 | 5 | 49 | 43 | 2 | 6 | 49 | 42 | 2 | 7 |
| 48 | 46 | 2 | 4 | 48 | 45 | 2 | 5 | 48 | 44 | 2 | 6 | 48 | 43 | 2 | 7 |
| 47 | 47 | 2 | 4 | 47 | 46 | 2 | 5 | 47 | 45 | 2 | 6 | 47 | 44 | 2 | 7 |
| 46 | 48 | 2 | 4 | 46 | 47 | 2 | 5 | 46 | 46 | 2 | 6 | 46 | 45 | 2 | 7 |
| 45 | 49 | 2 | 4 | 45 | 48 | 2 | 5 | 45 | 47 | 2 | 6 | 45 | 46 | 2 | 7 |
| 44 | 50 | 2 | 4 | 44 | 49 | 2 | 5 | 44 | 48 | 2 | 6 | 44 | 47 | 2 | 7 |
| 43 | 51 | 2 | 4 | 43 | 50 | 2 | 5 | 43 | 49 | 2 | 6 | 43 | 48 | 2 | 7 |
| 42 | 52 | 2 | 4 | 42 | 51 | 2 | 5 | 42 | 50 | 2 | 6 | 42 | 49 | 2 | 7 |
| 41 | 53 | 2 | 4 | 41 | 52 | 2 | 5 | 41 | 51 | 2 | 6 | 41 | 50 | 2 | 7 |
| 40 | 54 | 2 | 4 | 40 | 53 | 2 | 5 | 40 | 52 | 2 | 6 | 40 | 51 | 2 | 7 |
| 39 | 55 | 2 | 4 | 39 | 54 | 2 | 5 | 39 | 53 | 2 | 6 | 39 | 52 | 2 | 7 |
| 38 | 56 | 2 | 4 | 38 | 55 | 2 | 5 | 38 | 54 | 2 | 6 | 38 | 53 | 2 | 7 |
| 37 | 57 | 2 | 4 | 37 | 56 | 2 | 5 | 37 | 55 | 2 | 6 | 37 | 54 | 2 | 7 |
| 36 | 58 | 2 | 4 | 36 | 57 | 2 | 5 | 36 | 56 | 2 | 6 | 36 | 55 | 2 | 7 |
| 35 | 59 | 2 | 4 | 35 | 58 | 2 | 5 | 35 | 57 | 2 | 6 | 35 | 56 | 2 | 7 |
| 34 | 60 | 2 | 4 | 34 | 59 | 2 | 5 | 34 | 58 | 2 | 6 | 34 | 57 | 2 | 7 |
| 33 | 61 | 2 | 4 | 33 | 60 | 2 | 5 | 33 | 59 | 2 | 6 | 33 | 58 | 2 | 7 |
| 32 | 62 | 2 | 4 | 32 | 61 | 2 | 5 | 32 | 60 | 2 | 6 | 32 | 59 | 2 | 7 |
| 31 | 63 | 2 | 4 | 31 | 62 | 2 | 5 | 31 | 61 | 2 | 6 | 31 | 60 | 2 | 7 |
| 30 | 64 | 2 | 4 | 30 | 63 | 2 | 5 | 30 | 62 | 2 | 6 | 30 | 61 | 2 | 7 |
| 60 | 33.8 | 2.2 | 4 | 60 | 32.8 | 2.2 | 5 | 60 | 31.8 | 2.2 | 6 | 60 | 30.8 | 2.2 | 7 |
| 59 | 34.8 | 2.2 | 4 | 59 | 33.8 | 2.2 | 5 | 59 | 32.8 | 2.2 | 6 | 59 | 31.8 | 2.2 | 7 |
| 58 | 35.8 | 2.2 | 4 | 58 | 34.8 | 2.2 | 5 | 58 | 33.8 | 2.2 | 6 | 58 | 32.8 | 2.2 | 7 |
| 57 | 36.8 | 2.2 | 4 | 57 | 35.8 | 2.2 | 5 | 57 | 34.8 | 2.2 | 6 | 57 | 33.8 | 2.2 | 7 |
| 56 | 37.8 | 2.2 | 4 | 56 | 36.8 | 2.2 | 5 | 56 | 35.8 | 2.2 | 6 | 56 | 34.8 | 2.2 | 7 |
| 55 | 38.8 | 2.2 | 4 | 55 | 37.8 | 2.2 | 5 | 55 | 36.8 | 2.2 | 6 | 55 | 35.8 | 2.2 | 7 |
| 54 | 39.8 | 2.2 | 4 | 54 | 38.8 | 2.2 | 5 | 54 | 37.8 | 2.2 | 6 | 54 | 36.8 | 2.2 | 7 |
| 53 | 40.8 | 2.2 | 4 | 53 | 39.8 | 2.2 | 5 | 53 | 38.8 | 2.2 | 6 | 53 | 37.8 | 2.2 | 7 |
| 52 | 41.8 | 2.2 | 4 | 52 | 40.8 | 2.2 | 5 | 52 | 39.8 | 2.2 | 6 | 52 | 38.8 | 2.2 | 7 |
| 51 | 42.8 | 2.2 | 4 | 51 | 41.8 | 2.2 | 5 | 51 | 40.8 | 2.2 | 6 | 51 | 39.8 | 2.2 | 7 |
| 50 | 43.8 | 2.2 | 4 | 50 | 42.8 | 2.2 | 5 | 50 | 41.8 | 2.2 | 6 | 50 | 40.8 | 2.2 | 7 |
| 49 | 44.8 | 2.2 | 4 | 49 | 43.8 | 2.2 | 5 | 49 | 42.8 | 2.2 | 6 | 49 | 41.8 | 2.2 | 7 |
| 48 | 45.8 | 2.2 | 4 | 48 | 44.8 | 2.2 | 5 | 48 | 43.8 | 2.2 | 6 | 48 | 42.8 | 2.2 | 7 |
| 47 | 46.8 | 2.2 | 4 | 47 | 45.8 | 2.2 | 5 | 47 | 44.8 | 2.2 | 6 | 47 | 43.8 | 2.2 | 7 |
| 46 | 47.8 | 2.2 | 4 | 46 | 46.8 | 2.2 | 5 | 46 | 45.8 | 2.2 | 6 | 46 | 44.8 | 2.2 | 7 |
| 45 | 48.8 | 2.2 | 4 | 45 | 47.8 | 2.2 | 5 | 45 | 46.8 | 2.2 | 6 | 45 | 45.8 | 2.2 | 7 |
| 44 | 49.8 | 2.2 | 4 | 44 | 48.8 | 2.2 | 5 | 44 | 47.8 | 2.2 | 6 | 44 | 46.8 | 2.2 | 7 |
| 43 | 50.8 | 2.2 | 4 | 43 | 49.8 | 2.2 | 5 | 43 | 48.8 | 2.2 | 6 | 43 | 47.8 | 2.2 | 7 |
| 42 | 51.8 | 2.2 | 4 | 42 | 50.8 | 2.2 | 5 | 42 | 49.8 | 2.2 | 6 | 42 | 48.8 | 2.2 | 7 |
| 41 | 52.8 | 2.2 | 4 | 41 | 51.8 | 2.2 | 5 | 41 | 50.8 | 2.2 | 6 | 41 | 49.8 | 2.2 | 7 |
| 40 | 53.8 | 2.2 | 4 | 40 | 52.8 | 2.2 | 5 | 40 | 51.8 | 2.2 | 6 | 40 | 50.8 | 2.2 | 7 |
| 39 | 54.8 | 2.2 | 4 | 39 | 53.8 | 2.2 | 5 | 39 | 52.8 | 2.2 | 6 | 39 | 51.8 | 2.2 | 7 |
| 38 | 55.8 | 2.2 | 4 | 38 | 54.8 | 2.2 | 5 | 38 | 53.8 | 2.2 | 6 | 38 | 52.8 | 2.2 | 7 |
| 37 | 56.8 | 2.2 | 4 | 37 | 55.8 | 2.2 | 5 | 37 | 54.8 | 2.2 | 6 | 37 | 53.8 | 2.2 | 7 |
| 36 | 57.8 | 2.2 | 4 | 36 | 56.8 | 2.2 | 5 | 36 | 55.8 | 2.2 | 6 | 36 | 54.8 | 2.2 | 7 |
| 35 | 58.8 | 2.2 | 4 | 35 | 57.8 | 2.2 | 5 | 35 | 56.8 | 2.2 | 6 | 35 | 55.8 | 2.2 | 7 |
| 34 | 59.8 | 2.2 | 4 | 34 | 58.8 | 2.2 | 5 | 34 | 57.8 | 2.2 | 6 | 34 | 56.8 | 2.2 | 7 |
| 33 | 60.8 | 2.2 | 4 | 33 | 59.8 | 2.2 | 5 | 33 | 58.8 | 2.2 | 6 | 33 | 57.8 | 2.2 | 7 |
| 32 | 61.8 | 2.2 | 4 | 32 | 60.8 | 2.2 | 5 | 32 | 59.8 | 2.2 | 6 | 32 | 58.8 | 2.2 | 7 |
| 31 | 62.8 | 2.2 | 4 | 31 | 61.8 | 2.2 | 5 | 31 | 60.8 | 2.2 | 6 | 31 | 59.8 | 2.2 | 7 |
| 30 | 63.8 | 2.2 | 4 | 30 | 62.8 | 2.2 | 5 | 30 | 61.8 | 2.2 | 6 | 30 | 60.8 | 2.2 | 7 |
| 60 | 33.6 | 2.4 | 4 | 60 | 32.6 | 2.4 | 5 | 60 | 31.6 | 2.4 | 6 | 60 | 30.6 | 2.4 | 7 |
| 59 | 34.6 | 2.4 | 4 | 59 | 33.6 | 2.4 | 5 | 59 | 32.6 | 2.4 | 6 | 59 | 31.6 | 2.4 | 7 |
| 58 | 35.6 | 2.4 | 4 | 58 | 34.6 | 2.4 | 5 | 58 | 33.6 | 2.4 | 6 | 58 | 32.6 | 2.4 | 7 |
| 57 | 36.6 | 2.4 | 4 | 57 | 35.6 | 2.4 | 5 | 57 | 34.6 | 2.4 | 6 | 57 | 33.6 | 2.4 | 7 |
| 56 | 37.6 | 2.4 | 4 | 56 | 36.6 | 2.4 | 5 | 56 | 35.6 | 2.4 | 6 | 56 | 34.6 | 2.4 | 7 |
| 55 | 38.6 | 2.4 | 4 | 55 | 37.6 | 2.4 | 5 | 55 | 36.6 | 2.4 | 6 | 55 | 35.6 | 2.4 | 7 |
| 54 | 39.6 | 2.4 | 4 | 54 | 38.6 | 2.4 | 5 | 54 | 37.6 | 2.4 | 6 | 54 | 36.6 | 2.4 | 7 |
| 53 | 40.6 | 2.4 | 4 | 53 | 39.6 | 2.4 | 5 | 53 | 38.6 | 2.4 | 6 | 53 | 37.6 | 2.4 | 7 |
| 52 | 41.6 | 2.4 | 4 | 52 | 40.6 | 2.4 | 5 | 52 | 39.6 | 2.4 | 6 | 52 | 38.6 | 2.4 | 7 |
| 51 | 42.6 | 2.4 | 4 | 51 | 41.6 | 2.4 | 5 | 51 | 40.6 | 2.4 | 6 | 51 | 39.6 | 2.4 | 7 |
| 50 | 43.6 | 2.4 | 4 | 50 | 42.6 | 2.4 | 5 | 50 | 41.6 | 2.4 | 6 | 50 | 40.6 | 2.4 | 7 |
| 49 | 44.6 | 2.4 | 4 | 49 | 43.6 | 2.4 | 5 | 49 | 42.6 | 2.4 | 6 | 49 | 41.6 | 2.4 | 7 |
| 48 | 45.6 | 2.4 | 4 | 48 | 44.6 | 2.4 | 5 | 48 | 43.6 | 2.4 | 6 | 48 | 42.6 | 2.4 | 7 |
| 47 | 46.6 | 2.4 | 4 | 47 | 45.6 | 2.4 | 5 | 47 | 44.6 | 2.4 | 6 | 47 | 43.6 | 2.4 | 7 |
| 46 | 47.6 | 2.4 | 4 | 46 | 46.6 | 2.4 | 5 | 46 | 45.6 | 2.4 | 6 | 46 | 44.6 | 2.4 | 7 |
| 45 | 48.6 | 2.4 | 4 | 45 | 47.6 | 2.4 | 5 | 45 | 46.6 | 2.4 | 6 | 45 | 45.6 | 2.4 | 7 |
| 44 | 49.6 | 2.4 | 4 | 44 | 48.6 | 2.4 | 5 | 44 | 47.6 | 2.4 | 6 | 44 | 46.6 | 2.4 | 7 |
| 43 | 50.6 | 2.4 | 4 | 43 | 49.6 | 2.4 | 5 | 43 | 48.6 | 2.4 | 6 | 43 | 47.6 | 2.4 | 7 |
| 42 | 51.6 | 2.4 | 4 | 42 | 50.6 | 2.4 | 5 | 42 | 49.6 | 2.4 | 6 | 42 | 48.6 | 2.4 | 7 |
| 41 | 52.6 | 2.4 | 4 | 41 | 51.6 | 2.4 | 5 | 41 | 50.6 | 2.4 | 6 | 41 | 49.6 | 2.4 | 7 |
| 40 | 53.6 | 2.4 | 4 | 40 | 52.6 | 2.4 | 5 | 40 | 51.6 | 2.4 | 6 | 40 | 50.6 | 2.4 | 7 |
| 39 | 54.6 | 2.4 | 4 | 39 | 53.6 | 2.4 | 5 | 39 | 52.6 | 2.4 | 6 | 39 | 51.6 | 2.4 | 7 |
| 38 | 55.6 | 2.4 | 4 | 38 | 54.6 | 2.4 | 5 | 38 | 53.6 | 2.4 | 6 | 38 | 52.6 | 2.4 | 7 |
| 37 | 56.6 | 2.4 | 4 | 37 | 55.6 | 2.4 | 5 | 37 | 54.6 | 2.4 | 6 | 37 | 53.6 | 2.4 | 7 |
| 36 | 57.6 | 2.4 | 4 | 36 | 56.6 | 2.4 | 5 | 36 | 55.6 | 2.4 | 6 | 36 | 54.6 | 2.4 | 7 |
| 35 | 58.6 | 2.4 | 4 | 35 | 57.6 | 2.4 | 5 | 35 | 56.6 | 2.4 | 6 | 35 | 55.6 | 2.4 | 7 |
| 34 | 59.6 | 2.4 | 4 | 34 | 58.6 | 2.4 | 5 | 34 | 57.6 | 2.4 | 6 | 34 | 56.6 | 2.4 | 7 |
| 33 | 60.6 | 2.4 | 4 | 33 | 59.6 | 2.4 | 5 | 33 | 58.6 | 2.4 | 6 | 33 | 57.6 | 2.4 | 7 |
| 32 | 61.6 | 2.4 | 4 | 32 | 60.6 | 2.4 | 5 | 32 | 59.6 | 2.4 | 6 | 32 | 58.6 | 2.4 | 7 |
| 31 | 62.6 | 2.4 | 4 | 31 | 61.6 | 2.4 | 5 | 31 | 60.6 | 2.4 | 6 | 31 | 59.6 | 2.4 | 7 |
| 30 | 63.6 | 2.4 | 4 | 30 | 62.6 | 2.4 | 5 | 30 | 61.6 | 2.4 | 6 | 30 | 60.6 | 2.4 | 7 |
| 60 | 33.4 | 2.6 | 4 | 60 | 32.4 | 2.6 | 5 | 60 | 31.4 | 2.6 | 6 | 60 | 30.4 | 2.6 | 7 |
| 59 | 34.4 | 2.6 | 4 | 59 | 33.4 | 2.6 | 5 | 59 | 32.4 | 2.6 | 6 | 59 | 31.4 | 2.6 | 7 |
| 58 | 35.4 | 2.6 | 4 | 58 | 34.4 | 2.6 | 5 | 58 | 33.4 | 2.6 | 6 | 58 | 32.4 | 2.6 | 7 |
| 57 | 36.4 | 2.6 | 4 | 57 | 35.4 | 2.6 | 5 | 57 | 34.4 | 2.6 | 6 | 57 | 33.4 | 2.6 | 7 |
| 56 | 37.4 | 2.6 | 4 | 56 | 36.4 | 2.6 | 5 | 56 | 35.4 | 2.6 | 6 | 56 | 34.4 | 2.6 | 7 |
| 55 | 38.4 | 2.6 | 4 | 55 | 37.4 | 2.6 | 5 | 55 | 36.4 | 2.6 | 6 | 55 | 35.4 | 2.6 | 7 |
| 54 | 39.4 | 2.6 | 4 | 54 | 38.4 | 2.6 | 5 | 54 | 37.4 | 2.6 | 6 | 54 | 36.4 | 2.6 | 7 |
| 53 | 40.4 | 2.6 | 4 | 53 | 39.4 | 2.6 | 5 | 53 | 38.4 | 2.6 | 6 | 53 | 37.4 | 2.6 | 7 |
| 52 | 41.4 | 2.6 | 4 | 52 | 40.4 | 2.6 | 5 | 52 | 39.4 | 2.6 | 6 | 52 | 38.4 | 2.6 | 7 |
| 51 | 42.4 | 2.6 | 4 | 51 | 41.4 | 2.6 | 5 | 51 | 40.4 | 2.6 | 6 | 51 | 39.4 | 2.6 | 7 |
| 50 | 43.4 | 2.6 | 4 | 50 | 42.4 | 2.6 | 5 | 50 | 41.4 | 2.6 | 6 | 50 | 40.4 | 2.6 | 7 |
| 49 | 44.4 | 2.6 | 4 | 49 | 43.4 | 2.6 | 5 | 49 | 42.4 | 2.6 | 6 | 49 | 41.4 | 2.6 | 7 |
| 48 | 45.4 | 2.6 | 4 | 48 | 44.4 | 2.6 | 5 | 48 | 43.4 | 2.6 | 6 | 48 | 42.4 | 2.6 | 7 |
| 47 | 46.4 | 2.6 | 4 | 47 | 45.4 | 2.6 | 5 | 47 | 44.4 | 2.6 | 6 | 47 | 43.4 | 2.6 | 7 |
| 46 | 47.4 | 2.6 | 4 | 46 | 46.4 | 2.6 | 5 | 46 | 45.4 | 2.6 | 6 | 46 | 44.4 | 2.6 | 7 |
| 45 | 48.4 | 2.6 | 4 | 45 | 47.4 | 2.6 | 5 | 45 | 46.4 | 2.6 | 6 | 45 | 45.4 | 2.6 | 7 |
| 44 | 49.4 | 2.6 | 4 | 44 | 48.4 | 2.6 | 5 | 44 | 47.4 | 2.6 | 6 | 44 | 46.4 | 2.6 | 7 |
| 43 | 50.4 | 2.6 | 4 | 43 | 49.4 | 2.6 | 5 | 43 | 48.4 | 2.6 | 6 | 43 | 47.4 | 2.6 | 7 |
| 42 | 51.4 | 2.6 | 4 | 42 | 50.4 | 2.6 | 5 | 42 | 49.4 | 2.6 | 6 | 42 | 48.4 | 2.6 | 7 |
| 41 | 52.4 | 2.6 | 4 | 41 | 51.4 | 2.6 | 5 | 41 | 50.4 | 2.6 | 6 | 41 | 49.4 | 2.6 | 7 |
| 40 | 53.4 | 2.6 | 4 | 40 | 52.4 | 2.6 | 5 | 40 | 51.4 | 2.6 | 6 | 40 | 50.4 | 2.6 | 7 |
| 39 | 54.4 | 2.6 | 4 | 39 | 53.4 | 2.6 | 5 | 39 | 52.4 | 2.6 | 6 | 39 | 51.4 | 2.6 | 7 |
| 38 | 55.4 | 2.6 | 4 | 38 | 54.4 | 2.6 | 5 | 38 | 53.4 | 2.6 | 6 | 38 | 52.4 | 2.6 | 7 |
| 37 | 56.4 | 2.6 | 4 | 37 | 55.4 | 2.6 | 5 | 37 | 54.4 | 2.6 | 6 | 37 | 53.4 | 2.6 | 7 |
| 36 | 57.4 | 2.6 | 4 | 36 | 56.4 | 2.6 | 5 | 36 | 55.4 | 2.6 | 6 | 36 | 54.4 | 2.6 | 7 |
| 35 | 58.4 | 2.6 | 4 | 35 | 57.4 | 2.6 | 5 | 35 | 56.4 | 2.6 | 6 | 35 | 55.4 | 2.6 | 7 |
| 34 | 59.4 | 2.6 | 4 | 34 | 58.4 | 2.6 | 5 | 34 | 57.4 | 2.6 | 6 | 34 | 56.4 | 2.6 | 7 |
| 33 | 60.4 | 2.6 | 4 | 33 | 59.4 | 2.6 | 5 | 33 | 58.4 | 2.6 | 6 | 33 | 57.4 | 2.6 | 7 |
| 32 | 61.4 | 2.6 | 4 | 32 | 60.4 | 2.6 | 5 | 32 | 59.4 | 2.6 | 6 | 32 | 58.4 | 2.6 | 7 |
| 31 | 62.4 | 2.6 | 4 | 31 | 61.4 | 2.6 | 5 | 31 | 60.4 | 2.6 | 6 | 31 | 59.4 | 2.6 | 7 |
| 30 | 63.4 | 2.6 | 4 | 30 | 62.4 | 2.6 | 5 | 30 | 61.4 | 2.6 | 6 | 30 | 60.4 | 2.6 | 7 |
| 60 | 33.2 | 2.8 | 4 | 60 | 32.2 | 2.8 | 5 | 60 | 31.2 | 2.8 | 6 | 60 | 30.2 | 2.8 | 7 |
| 59 | 34.2 | 2.8 | 4 | 59 | 33.2 | 2.8 | 5 | 59 | 32.2 | 2.8 | 6 | 59 | 31.2 | 2.8 | 7 |
| 58 | 35.2 | 2.8 | 4 | 58 | 34.2 | 2.8 | 5 | 58 | 33.2 | 2.8 | 6 | 58 | 32.2 | 2.8 | 7 |
| 57 | 36.2 | 2.8 | 4 | 57 | 35.2 | 2.8 | 5 | 57 | 34.2 | 2.8 | 6 | 57 | 33.2 | 2.8 | 7 |
| 56 | 37.2 | 2.8 | 4 | 56 | 36.2 | 2.8 | 5 | 56 | 35.2 | 2.8 | 6 | 56 | 34.2 | 2.8 | 7 |
| 55 | 38.2 | 2.8 | 4 | 55 | 37.2 | 2.8 | 5 | 55 | 36.2 | 2.8 | 6 | 55 | 35.2 | 2.8 | 7 |
| 54 | 39.2 | 2.8 | 4 | 54 | 38.2 | 2.8 | 5 | 54 | 37.2 | 2.8 | 6 | 54 | 36.2 | 2.8 | 7 |
| 53 | 40.2 | 2.8 | 4 | 53 | 39.2 | 2.8 | 5 | 53 | 38.2 | 2.8 | 6 | 53 | 37.2 | 2.8 | 7 |
| 52 | 41.2 | 2.8 | 4 | 52 | 40.2 | 2.8 | 5 | 52 | 39.2 | 2.8 | 6 | 52 | 38.2 | 2.8 | 7 |
| 51 | 42.2 | 2.8 | 4 | 51 | 41.2 | 2.8 | 5 | 51 | 40.2 | 2.8 | 6 | 51 | 39.2 | 2.8 | 7 |
| 50 | 43.2 | 2.8 | 4 | 50 | 42.2 | 2.8 | 5 | 50 | 41.2 | 2.8 | 6 | 50 | 40.2 | 2.8 | 7 |
| 49 | 44.2 | 2.8 | 4 | 49 | 43.2 | 2.8 | 5 | 49 | 42.2 | 2.8 | 6 | 49 | 41.2 | 2.8 | 7 |
| 48 | 45.2 | 2.8 | 4 | 48 | 44.2 | 2.8 | 5 | 48 | 43.2 | 2.8 | 6 | 48 | 42.2 | 2.8 | 7 |
| 47 | 46.2 | 2.8 | 4 | 47 | 45.2 | 2.8 | 5 | 47 | 44.2 | 2.8 | 6 | 47 | 43.2 | 2.8 | 7 |
| 46 | 47.2 | 2.8 | 4 | 46 | 46.2 | 2.8 | 5 | 46 | 45.2 | 2.8 | 6 | 46 | 44.2 | 2.8 | 7 |
| 45 | 48.2 | 2.8 | 4 | 45 | 47.2 | 2.8 | 5 | 45 | 46.2 | 2.8 | 6 | 45 | 45.2 | 2.8 | 7 |
| 44 | 49.2 | 2.8 | 4 | 44 | 48.2 | 2.8 | 5 | 44 | 47.2 | 2.8 | 6 | 44 | 46.2 | 2.8 | 7 |
| 43 | 50.2 | 2.8 | 4 | 43 | 49.2 | 2.8 | 5 | 43 | 48.2 | 2.8 | 6 | 43 | 47.2 | 2.8 | 7 |
| 42 | 51.2 | 2.8 | 4 | 42 | 50.2 | 2.8 | 5 | 42 | 49.2 | 2.8 | 6 | 42 | 48.2 | 2.8 | 7 |
| 41 | 52.2 | 2.8 | 4 | 41 | 51.2 | 2.8 | 5 | 41 | 50.2 | 2.8 | 6 | 41 | 49.2 | 2.8 | 7 |
| 40 | 53.2 | 2.8 | 4 | 40 | 52.2 | 2.8 | 5 | 40 | 51.2 | 2.8 | 6 | 40 | 50.2 | 2.8 | 7 |
| 39 | 54.2 | 2.8 | 4 | 39 | 53.2 | 2.8 | 5 | 39 | 52.2 | 2.8 | 6 | 39 | 51.2 | 2.8 | 7 |
| 38 | 55.2 | 2.8 | 4 | 38 | 54.2 | 2.8 | 5 | 38 | 53.2 | 2.8 | 6 | 38 | 52.2 | 2.8 | 7 |
| 37 | 56.2 | 2.8 | 4 | 37 | 55.2 | 2.8 | 5 | 37 | 54.2 | 2.8 | 6 | 37 | 53.2 | 2.8 | 7 |
| 36 | 57.2 | 2.8 | 4 | 36 | 56.2 | 2.8 | 5 | 36 | 55.2 | 2.8 | 6 | 36 | 54.2 | 2.8 | 7 |
| 35 | 58.2 | 2.8 | 4 | 35 | 57.2 | 2.8 | 5 | 35 | 56.2 | 2.8 | 6 | 35 | 55.2 | 2.8 | 7 |
| 34 | 59.2 | 2.8 | 4 | 34 | 58.2 | 2.8 | 5 | 34 | 57.2 | 2.8 | 6 | 34 | 56.2 | 2.8 | 7 |
| 33 | 60.2 | 2.8 | 4 | 33 | 59.2 | 2.8 | 5 | 33 | 58.2 | 2.8 | 6 | 33 | 57.2 | 2.8 | 7 |
| 32 | 61.2 | 2.8 | 4 | 32 | 60.2 | 2.8 | 5 | 32 | 59.2 | 2.8 | 6 | 32 | 58.2 | 2.8 | 7 |
| 31 | 62.2 | 2.8 | 4 | 31 | 61.2 | 2.8 | 5 | 31 | 60.2 | 2.8 | 6 | 31 | 59.2 | 2.8 | 7 |
| 30 | 63.2 | 2.8 | 4 | 30 | 62.2 | 2.8 | 5 | 30 | 61.2 | 2.8 | 6 | 30 | 60.2 | 2.8 | 7 |
| 60 | 33 | 3 | 4 | 60 | 32 | 3 | 5 | 60 | 31 | 3 | 6 | 60 | 30 | 3 | 7 |
| 59 | 34 | 3 | 4 | 59 | 33 | 3 | 5 | 59 | 32 | 3 | 6 | 59 | 31 | 3 | 7 |
| 58 | 35 | 3 | 4 | 58 | 34 | 3 | 5 | 58 | 33 | 3 | 6 | 58 | 32 | 3 | 7 |
| 57 | 36 | 3 | 4 | 57 | 35 | 3 | 5 | 57 | 34 | 3 | 6 | 57 | 33 | 3 | 7 |
| 56 | 37 | 3 | 4 | 56 | 36 | 3 | 5 | 56 | 35 | 3 | 6 | 56 | 34 | 3 | 7 |
| 55 | 38 | 3 | 4 | 55 | 37 | 3 | 5 | 55 | 36 | 3 | 6 | 55 | 35 | 3 | 7 |
| 54 | 39 | 3 | 4 | 54 | 38 | 3 | 5 | 54 | 37 | 3 | 6 | 54 | 36 | 3 | 7 |
| 53 | 40 | 3 | 4 | 53 | 39 | 3 | 5 | 53 | 38 | 3 | 6 | 53 | 37 | 3 | 7 |
| 52 | 41 | 3 | 4 | 52 | 40 | 3 | 5 | 52 | 39 | 3 | 6 | 52 | 38 | 3 | 7 |
| 51 | 42 | 3 | 4 | 51 | 41 | 3 | 5 | 51 | 40 | 3 | 6 | 51 | 39 | 3 | 7 |
| 50 | 43 | 3 | 4 | 50 | 42 | 3 | 5 | 50 | 41 | 3 | 6 | 50 | 40 | 3 | 7 |
| 49 | 44 | 3 | 4 | 49 | 43 | 3 | 5 | 49 | 42 | 3 | 6 | 49 | 41 | 3 | 7 |
| 48 | 45 | 3 | 4 | 48 | 44 | 3 | 5 | 48 | 43 | 3 | 6 | 48 | 42 | 3 | 7 |
| 47 | 46 | 3 | 4 | 47 | 45 | 3 | 5 | 47 | 44 | 3 | 6 | 47 | 43 | 3 | 7 |
| 46 | 47 | 3 | 4 | 46 | 46 | 3 | 5 | 46 | 45 | 3 | 6 | 46 | 44 | 3 | 7 |
| 45 | 48 | 3 | 4 | 45 | 47 | 3 | 5 | 45 | 46 | 3 | 6 | 45 | 45 | 3 | 7 |
| 44 | 49 | 3 | 4 | 44 | 48 | 3 | 5 | 44 | 47 | 3 | 6 | 44 | 46 | 3 | 7 |
| 43 | 50 | 3 | 4 | 43 | 49 | 3 | 5 | 43 | 48 | 3 | 6 | 43 | 47 | 3 | 7 |
| 42 | 51 | 3 | 4 | 42 | 50 | 3 | 5 | 42 | 49 | 3 | 6 | 42 | 48 | 3 | 7 |
| 41 | 52 | 3 | 4 | 41 | 51 | 3 | 5 | 41 | 50 | 3 | 6 | 41 | 49 | 3 | 7 |
| 40 | 53 | 3 | 4 | 40 | 52 | 3 | 5 | 40 | 51 | 3 | 6 | 40 | 50 | 3 | 7 |
| 39 | 54 | 3 | 4 | 39 | 53 | 3 | 5 | 39 | 52 | 3 | 6 | 39 | 51 | 3 | 7 |
| 38 | 55 | 3 | 4 | 38 | 54 | 3 | 5 | 38 | 53 | 3 | 6 | 38 | 52 | 3 | 7 |
| 37 | 56 | 3 | 4 | 37 | 55 | 3 | 5 | 37 | 54 | 3 | 6 | 37 | 53 | 3 | 7 |
| 36 | 57 | 3 | 4 | 36 | 56 | 3 | 5 | 36 | 55 | 3 | 6 | 36 | 54 | 3 | 7 |
| 35 | 58 | 3 | 4 | 35 | 57 | 3 | 5 | 35 | 56 | 3 | 6 | 35 | 55 | 3 | 7 |
| 34 | 59 | 3 | 4 | 34 | 58 | 3 | 5 | 34 | 57 | 3 | 6 | 34 | 56 | 3 | 7 |
| 33 | 60 | 3 | 4 | 33 | 59 | 3 | 5 | 33 | 58 | 3 | 6 | 33 | 57 | 3 | 7 |
| 32 | 61 | 3 | 4 | 32 | 60 | 3 | 5 | 32 | 59 | 3 | 6 | 32 | 58 | 3 | 7 |
| 31 | 62 | 3 | 4 | 31 | 61 | 3 | 5 | 31 | 60 | 3 | 6 | 31 | 59 | 3 | 7 |
| 30 | 63 | 3 | 4 | 30 | 62 | 3 | 5 | 30 | 61 | 3 | 6 | 30 | 60 | 3 | 7 |
| 60 | 31 | 1 | 8 | 60 | 30 | 1 | 9 | 60 | 29 | 1 | 10 | 60 | 28 | 1 | 11 |
| 59 | 32 | 1 | 8 | 59 | 31 | 1 | 9 | 59 | 30 | 1 | 10 | 59 | 29 | 1 | 11 |
| 58 | 33 | 1 | 8 | 58 | 32 | 1 | 9 | 58 | 31 | 1 | 10 | 58 | 30 | 1 | 11 |
| 57 | 34 | 1 | 8 | 57 | 33 | 1 | 9 | 57 | 32 | 1 | 10 | 57 | 31 | 1 | 11 |
| 56 | 35 | 1 | 8 | 56 | 34 | 1 | 9 | 56 | 33 | 1 | 10 | 56 | 32 | 1 | 11 |
| 55 | 36 | 1 | 8 | 55 | 35 | 1 | 9 | 55 | 34 | 1 | 10 | 55 | 33 | 1 | 11 |
| 54 | 37 | 1 | 8 | 54 | 36 | 1 | 9 | 54 | 35 | 1 | 10 | 54 | 34 | 1 | 11 |
| 53 | 38 | 1 | 8 | 53 | 37 | 1 | 9 | 53 | 36 | 1 | 10 | 53 | 35 | 1 | 11 |
| 52 | 39 | 1 | 8 | 52 | 38 | 1 | 9 | 52 | 37 | 1 | 10 | 52 | 36 | 1 | 11 |
| 51 | 40 | 1 | 8 | 51 | 39 | 1 | 9 | 51 | 38 | 1 | 10 | 51 | 37 | 1 | 11 |
| 50 | 41 | 1 | 8 | 50 | 40 | 1 | 9 | 50 | 39 | 1 | 10 | 50 | 38 | 1 | 11 |
| 49 | 42 | 1 | 8 | 49 | 41 | 1 | 9 | 49 | 40 | 1 | 10 | 49 | 39 | 1 | 11 |
| 48 | 43 | 1 | 8 | 48 | 42 | 1 | 9 | 48 | 41 | 1 | 10 | 48 | 40 | 1 | 11 |
| 47 | 44 | 1 | 8 | 47 | 43 | 1 | 9 | 47 | 42 | 1 | 10 | 47 | 41 | 1 | 11 |
| 46 | 45 | 1 | 8 | 46 | 44 | 1 | 9 | 46 | 43 | 1 | 10 | 46 | 42 | 1 | 11 |
| 45 | 46 | 1 | 8 | 45 | 45 | 1 | 9 | 45 | 44 | 1 | 10 | 45 | 43 | 1 | 11 |
| 44 | 47 | 1 | 8 | 44 | 46 | 1 | 9 | 44 | 45 | 1 | 10 | 44 | 44 | 1 | 11 |
| 43 | 48 | 1 | 8 | 43 | 47 | 1 | 9 | 43 | 46 | 1 | 10 | 43 | 45 | 1 | 11 |
| 42 | 49 | 1 | 8 | 42 | 48 | 1 | 9 | 42 | 47 | 1 | 10 | 42 | 46 | 1 | 11 |
| 41 | 50 | 1 | 8 | 41 | 49 | 1 | 9 | 41 | 48 | 1 | 10 | 41 | 47 | 1 | 11 |
| 40 | 51 | 1 | 8 | 40 | 50 | 1 | 9 | 40 | 49 | 1 | 10 | 40 | 48 | 1 | 11 |
| 39 | 52 | 1 | 8 | 39 | 51 | 1 | 9 | 39 | 50 | 1 | 10 | 39 | 49 | 1 | 11 |
| 38 | 53 | 1 | 8 | 38 | 52 | 1 | 9 | 38 | 51 | 1 | 10 | 38 | 50 | 1 | 11 |
| 37 | 54 | 1 | 8 | 37 | 53 | 1 | 9 | 37 | 52 | 1 | 10 | 37 | 51 | 1 | 11 |
| 36 | 55 | 1 | 8 | 36 | 54 | 1 | 9 | 36 | 53 | 1 | 10 | 36 | 52 | 1 | 11 |
| 35 | 56 | 1 | 8 | 35 | 55 | 1 | 9 | 35 | 54 | 1 | 10 | 35 | 53 | 1 | 11 |
| 34 | 57 | 1 | 8 | 34 | 56 | 1 | 9 | 34 | 55 | 1 | 10 | 34 | 54 | 1 | 11 |
| 33 | 58 | 1 | 8 | 33 | 57 | 1 | 9 | 33 | 56 | 1 | 10 | 33 | 55 | 1 | 11 |
| 32 | 59 | 1 | 8 | 32 | 58 | 1 | 9 | 32 | 57 | 1 | 10 | 32 | 56 | 1 | 11 |
| 31 | 60 | 1 | 8 | 31 | 59 | 1 | 9 | 31 | 58 | 1 | 10 | 31 | 57 | 1 | 11 |
| 30 | 61 | 1 | 8 | 30 | 60 | 1 | 9 | 30 | 59 | 1 | 10 | 30 | 58 | 1 | 11 |
| 60 | 30.8 | 1.2 | 8 | 60 | 29.8 | 1.2 | 9 | 60 | 28.8 | 1.2 | 10 | 60 | 27.8 | 1.2 | 11 |
| 59 | 31.8 | 1.2 | 8 | 59 | 30.8 | 1.2 | 9 | 59 | 29.8 | 1.2 | 10 | 59 | 28.8 | 1.2 | 11 |
| 58 | 32.8 | 1.2 | 8 | 58 | 31.8 | 1.2 | 9 | 58 | 30.8 | 1.2 | 10 | 58 | 29.8 | 1.2 | 11 |
| 57 | 33.8 | 1.2 | 8 | 57 | 32.8 | 1.2 | 9 | 57 | 31.8 | 1.2 | 10 | 57 | 30.8 | 1.2 | 11 |
| 56 | 34.8 | 1.2 | 8 | 56 | 33.8 | 1.2 | 9 | 56 | 32.8 | 1.2 | 10 | 56 | 31.8 | 1.2 | 11 |
| 55 | 35.8 | 1.2 | 8 | 55 | 34.8 | 1.2 | 9 | 55 | 33.8 | 1.2 | 10 | 55 | 32.8 | 1.2 | 11 |
| 54 | 36.8 | 1.2 | 8 | 54 | 35.8 | 1.2 | 9 | 54 | 34.8 | 1.2 | 10 | 54 | 33.8 | 1.2 | 11 |
| 53 | 37.8 | 1.2 | 8 | 53 | 36.8 | 1.2 | 9 | 53 | 35.8 | 1.2 | 10 | 53 | 34.8 | 1.2 | 11 |
| 52 | 38.8 | 1.2 | 8 | 52 | 37.8 | 1.2 | 9 | 52 | 36.8 | 1.2 | 10 | 52 | 35.8 | 1.2 | 11 |
| 51 | 39.8 | 1.2 | 8 | 51 | 38.8 | 1.2 | 9 | 51 | 37.8 | 1.2 | 10 | 51 | 36.8 | 1.2 | 11 |
| 50 | 40.8 | 1.2 | 8 | 50 | 39.8 | 1.2 | 9 | 50 | 38.8 | 1.2 | 10 | 50 | 37.8 | 1.2 | 11 |
| 49 | 41.8 | 1.2 | 8 | 49 | 40.8 | 1.2 | 9 | 49 | 39.8 | 1.2 | 10 | 49 | 38.8 | 1.2 | 11 |
| 48 | 42.8 | 1.2 | 8 | 48 | 41.8 | 1.2 | 9 | 48 | 40.8 | 1.2 | 10 | 48 | 39.8 | 1.2 | 11 |
| 47 | 43.8 | 1.2 | 8 | 47 | 42.8 | 1.2 | 9 | 47 | 41.8 | 1.2 | 10 | 47 | 40.8 | 1.2 | 11 |
| 46 | 44.8 | 1.2 | 8 | 46 | 43.8 | 1.2 | 9 | 46 | 42.8 | 1.2 | 10 | 46 | 41.8 | 1.2 | 11 |
| 45 | 45.8 | 1.2 | 8 | 45 | 44.8 | 1.2 | 9 | 45 | 43.8 | 1.2 | 10 | 45 | 42.8 | 1.2 | 11 |
| 44 | 46.8 | 1.2 | 8 | 44 | 45.8 | 1.2 | 9 | 44 | 44.8 | 1.2 | 10 | 44 | 43.8 | 1.2 | 11 |
| 43 | 47.8 | 1.2 | 8 | 43 | 46.8 | 1.2 | 9 | 43 | 45.8 | 1.2 | 10 | 43 | 44.8 | 1.2 | 11 |
| 42 | 48.8 | 1.2 | 8 | 42 | 47.8 | 1.2 | 9 | 42 | 46.8 | 1.2 | 10 | 42 | 45.8 | 1.2 | 11 |
| 41 | 49.8 | 1.2 | 8 | 41 | 48.8 | 1.2 | 9 | 41 | 47.8 | 1.2 | 10 | 41 | 46.8 | 1.2 | 11 |
| 40 | 50.8 | 1.2 | 8 | 40 | 49.8 | 1.2 | 9 | 40 | 48.8 | 1.2 | 10 | 40 | 47.8 | 1.2 | 11 |
| 39 | 51.8 | 1.2 | 8 | 39 | 50.8 | 1.2 | 9 | 39 | 49.8 | 1.2 | 10 | 39 | 48.8 | 1.2 | 11 |
| 38 | 52.8 | 1.2 | 8 | 38 | 51.8 | 1.2 | 9 | 38 | 50.8 | 1.2 | 10 | 38 | 49.8 | 1.2 | 11 |
| 37 | 53.8 | 1.2 | 8 | 37 | 52.8 | 1.2 | 9 | 37 | 51.8 | 1.2 | 10 | 37 | 50.8 | 1.2 | 11 |
| 36 | 54.8 | 1.2 | 8 | 36 | 53.8 | 1.2 | 9 | 36 | 52.8 | 1.2 | 10 | 36 | 51.8 | 1.2 | 11 |
| 35 | 55.8 | 1.2 | 8 | 35 | 54.8 | 1.2 | 9 | 35 | 53.8 | 1.2 | 10 | 35 | 52.8 | 1.2 | 11 |
| 34 | 56.8 | 1.2 | 8 | 34 | 55.8 | 1.2 | 9 | 34 | 54.8 | 1.2 | 10 | 34 | 53.8 | 1.2 | 11 |
| 33 | 57.8 | 1.2 | 8 | 33 | 56.8 | 1.2 | 9 | 33 | 55.8 | 1.2 | 10 | 33 | 54.8 | 1.2 | 11 |
| 32 | 58.8 | 1.2 | 8 | 32 | 57.8 | 1.2 | 9 | 32 | 56.8 | 1.2 | 10 | 32 | 55.8 | 1.2 | 11 |
| 31 | 59.8 | 1.2 | 8 | 31 | 58.8 | 1.2 | 9 | 31 | 57.8 | 1.2 | 10 | 31 | 56.8 | 1.2 | 11 |
| 30 | 60.8 | 1.2 | 8 | 30 | 59.8 | 1.2 | 9 | 30 | 58.8 | 1.2 | 10 | 30 | 57.8 | 1.2 | 11 |
| 60 | 30.6 | 1.4 | 8 | 60 | 29.6 | 1.4 | 9 | 60 | 28.6 | 1.4 | 10 | 60 | 27.6 | 1.4 | 11 |
| 59 | 31.6 | 1.4 | 8 | 59 | 30.6 | 1.4 | 9 | 59 | 29.6 | 1.4 | 10 | 59 | 28.6 | 1.4 | 11 |
| 58 | 32.6 | 1.4 | 8 | 58 | 31.6 | 1.4 | 9 | 58 | 30.6 | 1.4 | 10 | 58 | 29.6 | 1.4 | 11 |
| 57 | 33.6 | 1.4 | 8 | 57 | 32.6 | 1.4 | 9 | 57 | 31.6 | 1.4 | 10 | 57 | 30.6 | 1.4 | 11 |
| 56 | 34.6 | 1.4 | 8 | 56 | 33.6 | 1.4 | 9 | 56 | 32.6 | 1.4 | 10 | 56 | 31.6 | 1.4 | 11 |
| 55 | 35.6 | 1.4 | 8 | 55 | 34.6 | 1.4 | 9 | 55 | 33.6 | 1.4 | 10 | 55 | 32.6 | 1.4 | 11 |
| 54 | 36.6 | 1.4 | 8 | 54 | 35.6 | 1.4 | 9 | 54 | 34.6 | 1.4 | 10 | 54 | 33.6 | 1.4 | 11 |
| 53 | 37.6 | 1.4 | 8 | 53 | 36.6 | 1.4 | 9 | 53 | 35.6 | 1.4 | 10 | 53 | 34.6 | 1.4 | 11 |
| 52 | 38.6 | 1.4 | 8 | 52 | 37.6 | 1.4 | 9 | 52 | 36.6 | 1.4 | 10 | 52 | 35.6 | 1.4 | 11 |
| 51 | 39.6 | 1.4 | 8 | 51 | 38.6 | 1.4 | 9 | 51 | 37.6 | 1.4 | 10 | 51 | 36.6 | 1.4 | 11 |
| 50 | 40.6 | 1.4 | 8 | 50 | 39.6 | 1.4 | 9 | 50 | 38.6 | 1.4 | 10 | 50 | 37.6 | 1.4 | 11 |
| 49 | 41.6 | 1.4 | 8 | 49 | 40.6 | 1.4 | 9 | 49 | 39.6 | 1.4 | 10 | 49 | 38.6 | 1.4 | 11 |
| 48 | 42.6 | 1.4 | 8 | 48 | 41.6 | 1.4 | 9 | 48 | 40.6 | 1.4 | 10 | 48 | 39.6 | 1.4 | 11 |
| 47 | 43.6 | 1.4 | 8 | 47 | 42.6 | 1.4 | 9 | 47 | 41.6 | 1.4 | 10 | 47 | 40.6 | 1.4 | 11 |
| 46 | 44.6 | 1.4 | 8 | 46 | 43.6 | 1.4 | 9 | 46 | 42.6 | 1.4 | 10 | 46 | 41.6 | 1.4 | 11 |
| 45 | 45.6 | 1.4 | 8 | 45 | 44.6 | 1.4 | 9 | 45 | 43.6 | 1.4 | 10 | 45 | 42.6 | 1.4 | 11 |
| 44 | 46.6 | 1.4 | 8 | 44 | 45.6 | 1.4 | 9 | 44 | 44.6 | 1.4 | 10 | 44 | 43.6 | 1.4 | 11 |
| 43 | 47.6 | 1.4 | 8 | 43 | 46.6 | 1.4 | 9 | 43 | 45.6 | 1.4 | 10 | 43 | 44.6 | 1.4 | 11 |
| 42 | 48.6 | 1.4 | 8 | 42 | 47.6 | 1.4 | 9 | 42 | 46.6 | 1.4 | 10 | 42 | 45.6 | 1.4 | 11 |
| 41 | 49.6 | 1.4 | 8 | 41 | 48.6 | 1.4 | 9 | 41 | 47.6 | 1.4 | 10 | 41 | 46.6 | 1.4 | 11 |
| 40 | 50.6 | 1.4 | 8 | 40 | 49.6 | 1.4 | 9 | 40 | 48.6 | 1.4 | 10 | 40 | 47.6 | 1.4 | 11 |
| 39 | 51.6 | 1.4 | 8 | 39 | 50.6 | 1.4 | 9 | 39 | 49.6 | 1.4 | 10 | 39 | 48.6 | 1.4 | 11 |
| 38 | 52.6 | 1.4 | 8 | 38 | 51.6 | 1.4 | 9 | 38 | 50.6 | 1.4 | 10 | 38 | 49.6 | 1.4 | 11 |
| 37 | 53.6 | 1.4 | 8 | 37 | 52.6 | 1.4 | 9 | 37 | 51.6 | 1.4 | 10 | 37 | 50.6 | 1.4 | 11 |
| 36 | 54.6 | 1.4 | 8 | 36 | 53.6 | 1.4 | 9 | 36 | 52.6 | 1.4 | 10 | 36 | 51.6 | 1.4 | 11 |
| 35 | 55.6 | 1.4 | 8 | 35 | 54.6 | 1.4 | 9 | 35 | 53.6 | 1.4 | 10 | 35 | 52.6 | 1.4 | 11 |
| 34 | 56.6 | 1.4 | 8 | 34 | 55.6 | 1.4 | 9 | 34 | 54.6 | 1.4 | 10 | 34 | 53.6 | 1.4 | 11 |
| 33 | 57.6 | 1.4 | 8 | 33 | 56.6 | 1.4 | 9 | 33 | 55.6 | 1.4 | 10 | 33 | 54.6 | 1.4 | 11 |
| 32 | 58.6 | 1.4 | 8 | 32 | 57.6 | 1.4 | 9 | 32 | 56.6 | 1.4 | 10 | 32 | 55.6 | 1.4 | 11 |
| 31 | 59.6 | 1.4 | 8 | 31 | 58.6 | 1.4 | 9 | 31 | 57.6 | 1.4 | 10 | 31 | 56.6 | 1.4 | 11 |
| 30 | 60.6 | 1.4 | 8 | 30 | 59.6 | 1.4 | 9 | 30 | 58.6 | 1.4 | 10 | 30 | 57.6 | 1.4 | 11 |
| 60 | 30.4 | 1.6 | 8 | 60 | 29.4 | 1.6 | 9 | 60 | 28.4 | 1.6 | 10 | 60 | 27.4 | 1.6 | 11 |
| 59 | 31.4 | 1.6 | 8 | 59 | 30.4 | 1.6 | 9 | 59 | 29.4 | 1.6 | 10 | 59 | 28.4 | 1.6 | 11 |
| 58 | 32.4 | 1.6 | 8 | 58 | 31.4 | 1.6 | 9 | 58 | 30.4 | 1.6 | 10 | 58 | 29.4 | 1.6 | 11 |
| 57 | 33.4 | 1.6 | 8 | 57 | 32.4 | 1.6 | 9 | 57 | 31.4 | 1.6 | 10 | 57 | 30.4 | 1.6 | 11 |
| 56 | 34.4 | 1.6 | 8 | 56 | 33.4 | 1.6 | 9 | 56 | 32.4 | 1.6 | 10 | 56 | 31.4 | 1.6 | 11 |
| 55 | 35.4 | 1.6 | 8 | 55 | 34.4 | 1.6 | 9 | 55 | 33.4 | 1.6 | 10 | 55 | 32.4 | 1.6 | 11 |
| 54 | 36.4 | 1.6 | 8 | 54 | 35.4 | 1.6 | 9 | 54 | 34.4 | 1.6 | 10 | 54 | 33.4 | 1.6 | 11 |
| 53 | 37.4 | 1.6 | 8 | 53 | 36.4 | 1.6 | 9 | 53 | 35.4 | 1.6 | 10 | 53 | 34.4 | 1.6 | 11 |
| 52 | 38.4 | 1.6 | 8 | 52 | 37.4 | 1.6 | 9 | 52 | 36.4 | 1.6 | 10 | 52 | 35.4 | 1.6 | 11 |
| 51 | 39.4 | 1.6 | 8 | 51 | 38.4 | 1.6 | 9 | 51 | 37.4 | 1.6 | 10 | 51 | 36.4 | 1.6 | 11 |
| 50 | 40.4 | 1.6 | 8 | 50 | 39.4 | 1.6 | 9 | 50 | 38.4 | 1.6 | 10 | 50 | 37.4 | 1.6 | 11 |
| 49 | 41.4 | 1.6 | 8 | 49 | 40.4 | 1.6 | 9 | 49 | 39.4 | 1.6 | 10 | 49 | 38.4 | 1.6 | 11 |
| 48 | 42.4 | 1.6 | 8 | 48 | 41.4 | 1.6 | 9 | 48 | 40.4 | 1.6 | 10 | 48 | 39.4 | 1.6 | 11 |
| 47 | 43.4 | 1.6 | 8 | 47 | 42.4 | 1.6 | 9 | 47 | 41.4 | 1.6 | 10 | 47 | 40.4 | 1.6 | 11 |
| 46 | 44.4 | 1.6 | 8 | 46 | 43.4 | 1.6 | 9 | 46 | 42.4 | 1.6 | 10 | 46 | 41.4 | 1.6 | 11 |
| 45 | 45.4 | 1.6 | 8 | 45 | 44.4 | 1.6 | 9 | 45 | 43.4 | 1.6 | 10 | 45 | 42.4 | 1.6 | 11 |
| 44 | 46.4 | 1.6 | 8 | 44 | 45.4 | 1.6 | 9 | 44 | 44.4 | 1.6 | 10 | 44 | 43.4 | 1.6 | 11 |
| 43 | 47.4 | 1.6 | 8 | 43 | 46.4 | 1.6 | 9 | 43 | 45.4 | 1.6 | 10 | 43 | 44.4 | 1.6 | 11 |
| 42 | 48.4 | 1.6 | 8 | 42 | 47.4 | 1.6 | 9 | 42 | 46.4 | 1.6 | 10 | 42 | 45.4 | 1.6 | 11 |
| 41 | 49.4 | 1.6 | 8 | 41 | 48.4 | 1.6 | 9 | 41 | 47.4 | 1.6 | 10 | 41 | 46.4 | 1.6 | 11 |
| 40 | 50.4 | 1.6 | 8 | 40 | 49.4 | 1.6 | 9 | 40 | 48.4 | 1.6 | 10 | 40 | 47.4 | 1.6 | 11 |
| 39 | 51.4 | 1.6 | 8 | 39 | 50.4 | 1.6 | 9 | 39 | 49.4 | 1.6 | 10 | 39 | 48.4 | 1.6 | 11 |
| 38 | 52.4 | 1.6 | 8 | 38 | 51.4 | 1.6 | 9 | 38 | 50.4 | 1.6 | 10 | 38 | 49.4 | 1.6 | 11 |
| 37 | 53.4 | 1.6 | 8 | 37 | 52.4 | 1.6 | 9 | 37 | 51.4 | 1.6 | 10 | 37 | 50.4 | 1.6 | 11 |
| 36 | 54.4 | 1.6 | 8 | 36 | 53.4 | 1.6 | 9 | 36 | 52.4 | 1.6 | 10 | 36 | 51.4 | 1.6 | 11 |
| 35 | 55.4 | 1.6 | 8 | 35 | 54.4 | 1.6 | 9 | 35 | 53.4 | 1.6 | 10 | 35 | 52.4 | 1.6 | 11 |
| 34 | 56.4 | 1.6 | 8 | 34 | 55.4 | 1.6 | 9 | 34 | 54.4 | 1.6 | 10 | 34 | 53.4 | 1.6 | 11 |
| 33 | 57.4 | 1.6 | 8 | 33 | 56.4 | 1.6 | 9 | 33 | 55.4 | 1.6 | 10 | 33 | 54.4 | 1.6 | 11 |
| 32 | 58.4 | 1.6 | 8 | 32 | 57.4 | 1.6 | 9 | 32 | 56.4 | 1.6 | 10 | 32 | 55.4 | 1.6 | 11 |
| 31 | 59.4 | 1.6 | 8 | 31 | 58.4 | 1.6 | 9 | 31 | 57.4 | 1.6 | 10 | 31 | 56.4 | 1.6 | 11 |
| 30 | 60.4 | 1.6 | 8 | 30 | 59.4 | 1.6 | 9 | 30 | 58.4 | 1.6 | 10 | 30 | 57.4 | 1.6 | 11 |
| 60 | 30.2 | 1.8 | 8 | 60 | 29.2 | 1.8 | 9 | 60 | 28.2 | 1.8 | 10 | 60 | 27.2 | 1.8 | 11 |
| 59 | 31.2 | 1.8 | 8 | 59 | 30.2 | 1.8 | 9 | 59 | 29.2 | 1.8 | 10 | 59 | 28.2 | 1.8 | 11 |
| 58 | 32.2 | 1.8 | 8 | 58 | 31.2 | 1.8 | 9 | 58 | 30.2 | 1.8 | 10 | 58 | 29.2 | 1.8 | 11 |
| 57 | 33.2 | 1.8 | 8 | 57 | 32.2 | 1.8 | 9 | 57 | 31.2 | 1.8 | 10 | 57 | 30.2 | 1.8 | 11 |
| 56 | 34.2 | 1.8 | 8 | 56 | 33.2 | 1.8 | 9 | 56 | 32.2 | 1.8 | 10 | 56 | 31.2 | 1.8 | 11 |
| 55 | 35.2 | 1.8 | 8 | 55 | 34.2 | 1.8 | 9 | 55 | 33.2 | 1.8 | 10 | 55 | 32.2 | 1.8 | 11 |
| 54 | 36.2 | 1.8 | 8 | 54 | 35.2 | 1.8 | 9 | 54 | 34.2 | 1.8 | 10 | 54 | 33.2 | 1.8 | 11 |
| 53 | 37.2 | 1.8 | 8 | 53 | 36.2 | 1.8 | 9 | 53 | 35.2 | 1.8 | 10 | 53 | 34.2 | 1.8 | 11 |
| 52 | 38.2 | 1.8 | 8 | 52 | 37.2 | 1.8 | 9 | 52 | 36.2 | 1.8 | 10 | 52 | 35.2 | 1.8 | 11 |
| 51 | 39.2 | 1.8 | 8 | 51 | 38.2 | 1.8 | 9 | 51 | 37.2 | 1.8 | 10 | 51 | 36.2 | 1.8 | 11 |
| 50 | 40.2 | 1.8 | 8 | 50 | 39.2 | 1.8 | 9 | 50 | 38.2 | 1.8 | 10 | 50 | 37.2 | 1.8 | 11 |
| 49 | 41.2 | 1.8 | 8 | 49 | 40.2 | 1.8 | 9 | 49 | 39.2 | 1.8 | 10 | 49 | 38.2 | 1.8 | 11 |
| 48 | 42.2 | 1.8 | 8 | 48 | 41.2 | 1.8 | 9 | 48 | 40.2 | 1.8 | 10 | 48 | 39.2 | 1.8 | 11 |
| 47 | 43.2 | 1.8 | 8 | 47 | 42.2 | 1.8 | 9 | 47 | 41.2 | 1.8 | 10 | 47 | 40.2 | 1.8 | 11 |
| 46 | 44.2 | 1.8 | 8 | 46 | 43.2 | 1.8 | 9 | 46 | 42.2 | 1.8 | 10 | 46 | 41.2 | 1.8 | 11 |
| 45 | 45.2 | 1.8 | 8 | 45 | 44.2 | 1.8 | 9 | 45 | 43.2 | 1.8 | 10 | 45 | 42.2 | 1.8 | 11 |
| 44 | 46.2 | 1.8 | 8 | 44 | 45.2 | 1.8 | 9 | 44 | 44.2 | 1.8 | 10 | 44 | 43.2 | 1.8 | 11 |
| 43 | 47.2 | 1.8 | 8 | 43 | 46.2 | 1.8 | 9 | 43 | 45.2 | 1.8 | 10 | 43 | 44.2 | 1.8 | 11 |
| 42 | 48.2 | 1.8 | 8 | 42 | 47.2 | 1.8 | 9 | 42 | 46.2 | 1.8 | 10 | 42 | 45.2 | 1.8 | 11 |
| 41 | 49.2 | 1.8 | 8 | 41 | 48.2 | 1.8 | 9 | 41 | 47.2 | 1.8 | 10 | 41 | 46.2 | 1.8 | 11 |
| 40 | 50.2 | 1.8 | 8 | 40 | 49.2 | 1.8 | 9 | 40 | 48.2 | 1.8 | 10 | 40 | 47.2 | 1.8 | 11 |
| 39 | 51.2 | 1.8 | 8 | 39 | 50.2 | 1.8 | 9 | 39 | 49.2 | 1.8 | 10 | 39 | 48.2 | 1.8 | 11 |
| 38 | 52.2 | 1.8 | 8 | 38 | 51.2 | 1.8 | 9 | 38 | 50.2 | 1.8 | 10 | 38 | 49.2 | 1.8 | 11 |
| 37 | 53.2 | 1.8 | 8 | 37 | 52.2 | 1.8 | 9 | 37 | 51.2 | 1.8 | 10 | 37 | 50.2 | 1.8 | 11 |
| 36 | 54.2 | 1.8 | 8 | 36 | 53.2 | 1.8 | 9 | 36 | 52.2 | 1.8 | 10 | 36 | 51.2 | 1.8 | 11 |
| 35 | 55.2 | 1.8 | 8 | 35 | 54.2 | 1.8 | 9 | 35 | 53.2 | 1.8 | 10 | 35 | 52.2 | 1.8 | 11 |
| 34 | 56.2 | 1.8 | 8 | 34 | 55.2 | 1.8 | 9 | 34 | 54.2 | 1.8 | 10 | 34 | 53.2 | 1.8 | 11 |
| 33 | 57.2 | 1.8 | 8 | 33 | 56.2 | 1.8 | 9 | 33 | 55.2 | 1.8 | 10 | 33 | 54.2 | 1.8 | 11 |
| 32 | 58.2 | 1.8 | 8 | 32 | 57.2 | 1.8 | 9 | 32 | 56.2 | 1.8 | 10 | 32 | 55.2 | 1.8 | 11 |
| 31 | 59.2 | 1.8 | 8 | 31 | 58.2 | 1.8 | 9 | 31 | 57.2 | 1.8 | 10 | 31 | 56.2 | 1.8 | 11 |
| 30 | 60.2 | 1.8 | 8 | 30 | 59.2 | 1.8 | 9 | 30 | 58.2 | 1.8 | 10 | 30 | 57.2 | 1.8 | 11 |
| 60 | 30 | 2 | 8 | 60 | 29 | 2 | 9 | 60 | 28 | 2 | 10 | 60 | 27 | 2 | 11 |
| 59 | 31 | 2 | 8 | 59 | 30 | 2 | 9 | 59 | 29 | 2 | 10 | 59 | 28 | 2 | 11 |
| 58 | 32 | 2 | 8 | 58 | 31 | 2 | 9 | 58 | 30 | 2 | 10 | 58 | 29 | 2 | 11 |
| 57 | 33 | 2 | 8 | 57 | 32 | 2 | 9 | 57 | 31 | 2 | 10 | 57 | 30 | 2 | 11 |
| 56 | 34 | 2 | 8 | 56 | 33 | 2 | 9 | 56 | 32 | 2 | 10 | 56 | 31 | 2 | 11 |
| 55 | 35 | 2 | 8 | 55 | 34 | 2 | 9 | 55 | 33 | 2 | 10 | 55 | 32 | 2 | 11 |
| 54 | 36 | 2 | 8 | 54 | 35 | 2 | 9 | 54 | 34 | 2 | 10 | 54 | 33 | 2 | 11 |
| 53 | 37 | 2 | 8 | 53 | 36 | 2 | 9 | 53 | 35 | 2 | 10 | 53 | 34 | 2 | 11 |
| 52 | 38 | 2 | 8 | 52 | 37 | 2 | 9 | 52 | 36 | 2 | 10 | 52 | 35 | 2 | 11 |
| 51 | 39 | 2 | 8 | 51 | 38 | 2 | 9 | 51 | 37 | 2 | 10 | 51 | 36 | 2 | 11 |
| 50 | 40 | 2 | 8 | 50 | 39 | 2 | 9 | 50 | 38 | 2 | 10 | 50 | 37 | 2 | 11 |
| 49 | 41 | 2 | 8 | 49 | 40 | 2 | 9 | 49 | 39 | 2 | 10 | 49 | 38 | 2 | 11 |
| 48 | 42 | 2 | 8 | 48 | 41 | 2 | 9 | 48 | 40 | 2 | 10 | 48 | 39 | 2 | 11 |
| 47 | 43 | 2 | 8 | 47 | 42 | 2 | 9 | 47 | 41 | 2 | 10 | 47 | 40 | 2 | 11 |
| 46 | 44 | 2 | 8 | 46 | 43 | 2 | 9 | 46 | 42 | 2 | 10 | 46 | 41 | 2 | 11 |
| 45 | 45 | 2 | 8 | 45 | 44 | 2 | 9 | 45 | 43 | 2 | 10 | 45 | 42 | 2 | 11 |
| 44 | 46 | 2 | 8 | 44 | 45 | 2 | 9 | 44 | 44 | 2 | 10 | 44 | 43 | 2 | 11 |
| 43 | 47 | 2 | 8 | 43 | 46 | 2 | 9 | 43 | 45 | 2 | 10 | 43 | 44 | 2 | 11 |
| 42 | 48 | 2 | 8 | 42 | 47 | 2 | 9 | 42 | 46 | 2 | 10 | 42 | 45 | 2 | 11 |
| 41 | 49 | 2 | 8 | 41 | 48 | 2 | 9 | 41 | 47 | 2 | 10 | 41 | 46 | 2 | 11 |
| 40 | 50 | 2 | 8 | 40 | 49 | 2 | 9 | 40 | 48 | 2 | 10 | 40 | 47 | 2 | 11 |
| 39 | 51 | 2 | 8 | 39 | 50 | 2 | 9 | 39 | 49 | 2 | 10 | 39 | 48 | 2 | 11 |
| 38 | 52 | 2 | 8 | 38 | 51 | 2 | 9 | 38 | 50 | 2 | 10 | 38 | 49 | 2 | 11 |
| 37 | 53 | 2 | 8 | 37 | 52 | 2 | 9 | 37 | 51 | 2 | 10 | 37 | 50 | 2 | 11 |
| 36 | 54 | 2 | 8 | 36 | 53 | 2 | 9 | 36 | 52 | 2 | 10 | 36 | 51 | 2 | 11 |
| 35 | 55 | 2 | 8 | 35 | 54 | 2 | 9 | 35 | 53 | 2 | 10 | 35 | 52 | 2 | 11 |
| 34 | 56 | 2 | 8 | 34 | 55 | 2 | 9 | 34 | 54 | 2 | 10 | 34 | 53 | 2 | 11 |
| 33 | 57 | 2 | 8 | 33 | 56 | 2 | 9 | 33 | 55 | 2 | 10 | 33 | 54 | 2 | 11 |
| 32 | 58 | 2 | 8 | 32 | 57 | 2 | 9 | 32 | 56 | 2 | 10 | 32 | 55 | 2 | 11 |
| 31 | 59 | 2 | 8 | 31 | 58 | 2 | 9 | 31 | 57 | 2 | 10 | 31 | 56 | 2 | 11 |
| 30 | 60 | 2 | 8 | 30 | 59 | 2 | 9 | 30 | 58 | 2 | 10 | 30 | 57 | 2 | 11 |
| 60 | 29.8 | 2.2 | 8 | 60 | 28.8 | 2.2 | 9 | 60 | 27.8 | 2.2 | 10 | 60 | 26.8 | 2.2 | 11 |
| 59 | 30.8 | 2.2 | 8 | 59 | 29.8 | 2.2 | 9 | 59 | 28.8 | 2.2 | 10 | 59 | 27.8 | 2.2 | 11 |
| 58 | 31.8 | 2.2 | 8 | 58 | 30.8 | 2.2 | 9 | 58 | 29.8 | 2.2 | 10 | 58 | 28.8 | 2.2 | 11 |
| 57 | 32.8 | 2.2 | 8 | 57 | 31.8 | 2.2 | 9 | 57 | 30.8 | 2.2 | 10 | 57 | 29.8 | 2.2 | 11 |
| 56 | 33.8 | 2.2 | 8 | 56 | 32.8 | 2.2 | 9 | 56 | 31.8 | 2.2 | 10 | 56 | 30.8 | 2.2 | 11 |
| 55 | 34.8 | 2.2 | 8 | 55 | 33.8 | 2.2 | 9 | 55 | 32.8 | 2.2 | 10 | 55 | 31.8 | 2.2 | 11 |
| 54 | 35.8 | 2.2 | 8 | 54 | 34.8 | 2.2 | 9 | 54 | 33.8 | 2.2 | 10 | 54 | 32.8 | 2.2 | 11 |
| 53 | 36.8 | 2.2 | 8 | 53 | 35.8 | 2.2 | 9 | 53 | 34.8 | 2.2 | 10 | 53 | 33.8 | 2.2 | 11 |
| 52 | 37.8 | 2.2 | 8 | 52 | 36.8 | 2.2 | 9 | 52 | 35.8 | 2.2 | 10 | 52 | 34.8 | 2.2 | 11 |
| 51 | 38.8 | 2.2 | 8 | 51 | 37.8 | 2.2 | 9 | 51 | 36.8 | 2.2 | 10 | 51 | 35.8 | 2.2 | 11 |
| 50 | 39.8 | 2.2 | 8 | 50 | 38.8 | 2.2 | 9 | 50 | 37.8 | 2.2 | 10 | 50 | 36.8 | 2.2 | 11 |
| 49 | 40.8 | 2.2 | 8 | 49 | 39.8 | 2.2 | 9 | 49 | 38.8 | 2.2 | 10 | 49 | 37.8 | 2.2 | 11 |
| 48 | 41.8 | 2.2 | 8 | 48 | 40.8 | 2.2 | 9 | 48 | 39.8 | 2.2 | 10 | 48 | 38.8 | 2.2 | 11 |
| 47 | 42.8 | 2.2 | 8 | 47 | 41.8 | 2.2 | 9 | 47 | 40.8 | 2.2 | 10 | 47 | 39.8 | 2.2 | 11 |
| 46 | 43.8 | 2.2 | 8 | 46 | 42.8 | 2.2 | 9 | 46 | 41.8 | 2.2 | 10 | 46 | 40.8 | 2.2 | 11 |
| 45 | 44.8 | 2.2 | 8 | 45 | 43.8 | 2.2 | 9 | 45 | 42.8 | 2.2 | 10 | 45 | 41.8 | 2.2 | 11 |
| 44 | 45.8 | 2.2 | 8 | 44 | 44.8 | 2.2 | 9 | 44 | 43.8 | 2.2 | 10 | 44 | 42.8 | 2.2 | 11 |
| 43 | 46.8 | 2.2 | 8 | 43 | 45.8 | 2.2 | 9 | 43 | 44.8 | 2.2 | 10 | 43 | 43.8 | 2.2 | 11 |
| 42 | 47.8 | 2.2 | 8 | 42 | 46.8 | 2.2 | 9 | 42 | 45.8 | 2.2 | 10 | 42 | 44.8 | 2.2 | 11 |
| 41 | 48.8 | 2.2 | 8 | 41 | 47.8 | 2.2 | 9 | 41 | 46.8 | 2.2 | 10 | 41 | 45.8 | 2.2 | 11 |
| 40 | 49.8 | 2.2 | 8 | 40 | 48.8 | 2.2 | 9 | 40 | 47.8 | 2.2 | 10 | 40 | 46.8 | 2.2 | 11 |
| 39 | 50.8 | 2.2 | 8 | 39 | 49.8 | 2.2 | 9 | 39 | 48.8 | 2.2 | 10 | 39 | 47.8 | 2.2 | 11 |
| 38 | 51.8 | 2.2 | 8 | 38 | 50.8 | 2.2 | 9 | 38 | 49.8 | 2.2 | 10 | 38 | 48.8 | 2.2 | 11 |
| 37 | 52.8 | 2.2 | 8 | 37 | 51.8 | 2.2 | 9 | 37 | 50.8 | 2.2 | 10 | 37 | 49.8 | 2.2 | 11 |
| 36 | 53.8 | 2.2 | 8 | 36 | 52.8 | 2.2 | 9 | 36 | 51.8 | 2.2 | 10 | 36 | 50.8 | 2.2 | 11 |
| 35 | 54.8 | 2.2 | 8 | 35 | 53.8 | 2.2 | 9 | 35 | 52.8 | 2.2 | 10 | 35 | 51.8 | 2.2 | 11 |
| 34 | 55.8 | 2.2 | 8 | 34 | 54.8 | 2.2 | 9 | 34 | 53.8 | 2.2 | 10 | 34 | 52.8 | 2.2 | 11 |
| 33 | 56.8 | 2.2 | 8 | 33 | 55.8 | 2.2 | 9 | 33 | 54.8 | 2.2 | 10 | 33 | 53.8 | 2.2 | 11 |
| 32 | 57.8 | 2.2 | 8 | 32 | 56.8 | 2.2 | 9 | 32 | 55.8 | 2.2 | 10 | 32 | 54.8 | 2.2 | 11 |
| 31 | 58.8 | 2.2 | 8 | 31 | 57.8 | 2.2 | 9 | 31 | 56.8 | 2.2 | 10 | 31 | 55.8 | 2.2 | 11 |
| 30 | 59.8 | 2.2 | 8 | 30 | 58.8 | 2.2 | 9 | 30 | 57.8 | 2.2 | 10 | 30 | 56.8 | 2.2 | 11 |
| 60 | 29.6 | 2.4 | 8 | 60 | 28.6 | 2.4 | 9 | 60 | 27.6 | 2.4 | 10 | 60 | 26.6 | 2.4 | 11 |
| 59 | 30.6 | 2.4 | 8 | 59 | 29.6 | 2.4 | 9 | 59 | 28.6 | 2.4 | 10 | 59 | 27.6 | 2.4 | 11 |
| 58 | 31.6 | 2.4 | 8 | 58 | 30.6 | 2.4 | 9 | 58 | 29.6 | 2.4 | 10 | 58 | 28.6 | 2.4 | 11 |
| 57 | 32.6 | 2.4 | 8 | 57 | 31.6 | 2.4 | 9 | 57 | 30.6 | 2.4 | 10 | 57 | 29.6 | 2.4 | 11 |
| 56 | 33.6 | 2.4 | 8 | 56 | 32.6 | 2.4 | 9 | 56 | 31.6 | 2.4 | 10 | 56 | 30.6 | 2.4 | 11 |
| 55 | 34.6 | 2.4 | 8 | 55 | 33.6 | 2.4 | 9 | 55 | 32.6 | 2.4 | 10 | 55 | 31.6 | 2.4 | 11 |
| 54 | 35.6 | 2.4 | 8 | 54 | 34.6 | 2.4 | 9 | 54 | 33.6 | 2.4 | 10 | 54 | 32.6 | 2.4 | 11 |
| 53 | 36.6 | 2.4 | 8 | 53 | 35.6 | 2.4 | 9 | 53 | 34.6 | 2.4 | 10 | 53 | 33.6 | 2.4 | 11 |
| 52 | 37.6 | 2.4 | 8 | 52 | 36.6 | 2.4 | 9 | 52 | 35.6 | 2.4 | 10 | 52 | 34.6 | 2.4 | 11 |
| 51 | 38.6 | 2.4 | 8 | 51 | 37.6 | 2.4 | 9 | 51 | 36.6 | 2.4 | 10 | 51 | 35.6 | 2.4 | 11 |
| 50 | 39.6 | 2.4 | 8 | 50 | 38.6 | 2.4 | 9 | 50 | 37.6 | 2.4 | 10 | 50 | 36.6 | 2.4 | 11 |
| 49 | 40.6 | 2.4 | 8 | 49 | 39.6 | 2.4 | 9 | 49 | 38.6 | 2.4 | 10 | 49 | 37.6 | 2.4 | 11 |
| 48 | 41.6 | 2.4 | 8 | 48 | 40.6 | 2.4 | 9 | 48 | 39.6 | 2.4 | 10 | 48 | 38.6 | 2.4 | 11 |
| 47 | 42.6 | 2.4 | 8 | 47 | 41.6 | 2.4 | 9 | 47 | 40.6 | 2.4 | 10 | 47 | 39.6 | 2.4 | 11 |
| 46 | 43.6 | 2.4 | 8 | 46 | 42.6 | 2.4 | 9 | 46 | 41.6 | 2.4 | 10 | 46 | 40.6 | 2.4 | 11 |
| 45 | 44.6 | 2.4 | 8 | 45 | 43.6 | 2.4 | 9 | 45 | 42.6 | 2.4 | 10 | 45 | 41.6 | 2.4 | 11 |
| 44 | 45.6 | 2.4 | 8 | 44 | 44.6 | 2.4 | 9 | 44 | 43.6 | 2.4 | 10 | 44 | 42.6 | 2.4 | 11 |
| 43 | 46.6 | 2.4 | 8 | 43 | 45.6 | 2.4 | 9 | 43 | 44.6 | 2.4 | 10 | 43 | 43.6 | 2.4 | 11 |
| 42 | 47.6 | 2.4 | 8 | 42 | 46.6 | 2.4 | 9 | 42 | 45.6 | 2.4 | 10 | 42 | 44.6 | 2.4 | 11 |
| 41 | 48.6 | 2.4 | 8 | 41 | 47.6 | 2.4 | 9 | 41 | 46.6 | 2.4 | 10 | 41 | 45.6 | 2.4 | 11 |
| 40 | 49.6 | 2.4 | 8 | 40 | 48.6 | 2.4 | 9 | 40 | 47.6 | 2.4 | 10 | 40 | 46.6 | 2.4 | 11 |
| 39 | 50.6 | 2.4 | 8 | 39 | 49.6 | 2.4 | 9 | 39 | 48.6 | 2.4 | 10 | 39 | 47.6 | 2.4 | 11 |
| 38 | 51.6 | 2.4 | 8 | 38 | 50.6 | 2.4 | 9 | 38 | 49.6 | 2.4 | 10 | 38 | 48.6 | 2.4 | 11 |
| 37 | 52.6 | 2.4 | 8 | 37 | 51.6 | 2.4 | 9 | 37 | 50.6 | 2.4 | 10 | 37 | 49.6 | 2.4 | 11 |
| 36 | 53.6 | 2.4 | 8 | 36 | 52.6 | 2.4 | 9 | 36 | 51.6 | 2.4 | 10 | 36 | 50.6 | 2.4 | 11 |
| 35 | 54.6 | 2.4 | 8 | 35 | 53.6 | 2.4 | 9 | 35 | 52.6 | 2.4 | 10 | 35 | 51.6 | 2.4 | 11 |
| 34 | 55.6 | 2.4 | 8 | 34 | 54.6 | 2.4 | 9 | 34 | 53.6 | 2.4 | 10 | 34 | 52.6 | 2.4 | 11 |
| 33 | 56.6 | 2.4 | 8 | 33 | 55.6 | 2.4 | 9 | 33 | 54.6 | 2.4 | 10 | 33 | 53.6 | 2.4 | 11 |
| 32 | 57.6 | 2.4 | 8 | 32 | 56.6 | 2.4 | 9 | 32 | 55.6 | 2.4 | 10 | 32 | 54.6 | 2.4 | 11 |
| 31 | 58.6 | 2.4 | 8 | 31 | 57.6 | 2.4 | 9 | 31 | 56.6 | 2.4 | 10 | 31 | 55.6 | 2.4 | 11 |
| 30 | 59.6 | 2.4 | 8 | 30 | 58.6 | 2.4 | 9 | 30 | 57.6 | 2.4 | 10 | 30 | 56.6 | 2.4 | 11 |
| 60 | 29.4 | 2.6 | 8 | 60 | 28.4 | 2.6 | 9 | 60 | 27.4 | 2.6 | 10 | 60 | 26.4 | 2.6 | 11 |
| 59 | 30.4 | 2.6 | 8 | 59 | 29.4 | 2.6 | 9 | 59 | 28.4 | 2.6 | 10 | 59 | 27.4 | 2.6 | 11 |
| 58 | 31.4 | 2.6 | 8 | 58 | 30.4 | 2.6 | 9 | 58 | 29.4 | 2.6 | 10 | 58 | 28.4 | 2.6 | 11 |
| 57 | 32.4 | 2.6 | 8 | 57 | 31.4 | 2.6 | 9 | 57 | 30.4 | 2.6 | 10 | 57 | 29.4 | 2.6 | 11 |
| 56 | 33.4 | 2.6 | 8 | 56 | 32.4 | 2.6 | 9 | 56 | 31.4 | 2.6 | 10 | 56 | 30.4 | 2.6 | 11 |
| 55 | 34.4 | 2.6 | 8 | 55 | 33.4 | 2.6 | 9 | 55 | 32.4 | 2.6 | 10 | 55 | 31.4 | 2.6 | 11 |
| 54 | 35.4 | 2.6 | 8 | 54 | 34.4 | 2.6 | 9 | 54 | 33.4 | 2.6 | 10 | 54 | 32.4 | 2.6 | 11 |
| 53 | 36.4 | 2.6 | 8 | 53 | 35.4 | 2.6 | 9 | 53 | 34.4 | 2.6 | 10 | 53 | 33.4 | 2.6 | 11 |
| 52 | 37.4 | 2.6 | 8 | 52 | 36.4 | 2.6 | 9 | 52 | 35.4 | 2.6 | 10 | 52 | 34.4 | 2.6 | 11 |
| 51 | 38.4 | 2.6 | 8 | 51 | 37.4 | 2.6 | 9 | 51 | 36.4 | 2.6 | 10 | 51 | 35.4 | 2.6 | 11 |
| 50 | 39.4 | 2.6 | 8 | 50 | 38.4 | 2.6 | 9 | 50 | 37.4 | 2.6 | 10 | 50 | 36.4 | 2.6 | 11 |
| 49 | 40.4 | 2.6 | 8 | 49 | 39.4 | 2.6 | 9 | 49 | 38.4 | 2.6 | 10 | 49 | 37.4 | 2.6 | 11 |
| 48 | 41.4 | 2.6 | 8 | 48 | 40.4 | 2.6 | 9 | 48 | 39.4 | 2.6 | 10 | 48 | 38.4 | 2.6 | 11 |
| 47 | 42.4 | 2.6 | 8 | 47 | 41.4 | 2.6 | 9 | 47 | 40.4 | 2.6 | 10 | 47 | 39.4 | 2.6 | 11 |
| 46 | 43.4 | 2.6 | 8 | 46 | 42.4 | 2.6 | 9 | 46 | 41.4 | 2.6 | 10 | 46 | 40.4 | 2.6 | 11 |
| 45 | 44.4 | 2.6 | 8 | 45 | 43.4 | 2.6 | 9 | 45 | 42.4 | 2.6 | 10 | 45 | 41.4 | 2.6 | 11 |
| 44 | 45.4 | 2.6 | 8 | 44 | 44.4 | 2.6 | 9 | 44 | 43.4 | 2.6 | 10 | 44 | 42.4 | 2.6 | 11 |
| 43 | 46.4 | 2.6 | 8 | 43 | 45.4 | 2.6 | 9 | 43 | 44.4 | 2.6 | 10 | 43 | 43.4 | 2.6 | 11 |
| 42 | 47.4 | 2.6 | 8 | 42 | 46.4 | 2.6 | 9 | 42 | 45.4 | 2.6 | 10 | 42 | 44.4 | 2.6 | 11 |
| 41 | 48.4 | 2.6 | 8 | 41 | 47.4 | 2.6 | 9 | 41 | 46.4 | 2.6 | 10 | 41 | 45.4 | 2.6 | 11 |
| 40 | 49.4 | 2.6 | 8 | 40 | 48.4 | 2.6 | 9 | 40 | 47.4 | 2.6 | 10 | 40 | 46.4 | 2.6 | 11 |
| 39 | 50.4 | 2.6 | 8 | 39 | 49.4 | 2.6 | 9 | 39 | 48.4 | 2.6 | 10 | 39 | 47.4 | 2.6 | 11 |
| 38 | 51.4 | 2.6 | 8 | 38 | 50.4 | 2.6 | 9 | 38 | 49.4 | 2.6 | 10 | 38 | 48.4 | 2.6 | 11 |
| 37 | 52.4 | 2.6 | 8 | 37 | 51.4 | 2.6 | 9 | 37 | 50.4 | 2.6 | 10 | 37 | 49.4 | 2.6 | 11 |
| 36 | 53.4 | 2.6 | 8 | 36 | 52.4 | 2.6 | 9 | 36 | 51.4 | 2.6 | 10 | 36 | 50.4 | 2.6 | 11 |
| 35 | 54.4 | 2.6 | 8 | 35 | 53.4 | 2.6 | 9 | 35 | 52.4 | 2.6 | 10 | 35 | 51.4 | 2.6 | 11 |
| 34 | 55.4 | 2.6 | 8 | 34 | 54.4 | 2.6 | 9 | 34 | 53.4 | 2.6 | 10 | 34 | 52.4 | 2.6 | 11 |
| 33 | 56.4 | 2.6 | 8 | 33 | 55.4 | 2.6 | 9 | 33 | 54.4 | 2.6 | 10 | 33 | 53.4 | 2.6 | 11 |
| 32 | 57.4 | 2.6 | 8 | 32 | 56.4 | 2.6 | 9 | 32 | 55.4 | 2.6 | 10 | 32 | 54.4 | 2.6 | 11 |
| 31 | 58.4 | 2.6 | 8 | 31 | 57.4 | 2.6 | 9 | 31 | 56.4 | 2.6 | 10 | 31 | 55.4 | 2.6 | 11 |
| 30 | 59.4 | 2.6 | 8 | 30 | 58.4 | 2.6 | 9 | 30 | 57.4 | 2.6 | 10 | 30 | 56.4 | 2.6 | 11 |
| 60 | 29.2 | 2.8 | 8 | 60 | 28.2 | 2.8 | 9 | 60 | 27.2 | 2.8 | 10 | 60 | 26.2 | 2.8 | 11 |
| 59 | 30.2 | 2.8 | 8 | 59 | 29.2 | 2.8 | 9 | 59 | 28.2 | 2.8 | 10 | 59 | 27.2 | 2.8 | 11 |
| 58 | 31.2 | 2.8 | 8 | 58 | 30.2 | 2.8 | 9 | 58 | 29.2 | 2.8 | 10 | 58 | 28.2 | 2.8 | 11 |
| 57 | 32.2 | 2.8 | 8 | 57 | 31.2 | 2.8 | 9 | 57 | 30.2 | 2.8 | 10 | 57 | 29.2 | 2.8 | 11 |
| 56 | 33.2 | 2.8 | 8 | 56 | 32.2 | 2.8 | 9 | 56 | 31.2 | 2.8 | 10 | 56 | 30.2 | 2.8 | 11 |
| 55 | 34.2 | 2.8 | 8 | 55 | 33.2 | 2.8 | 9 | 55 | 32.2 | 2.8 | 10 | 55 | 31.2 | 2.8 | 11 |
| 54 | 35.2 | 2.8 | 8 | 54 | 34.2 | 2.8 | 9 | 54 | 33.2 | 2.8 | 10 | 54 | 32.2 | 2.8 | 11 |
| 53 | 36.2 | 2.8 | 8 | 53 | 35.2 | 2.8 | 9 | 53 | 34.2 | 2.8 | 10 | 53 | 33.2 | 2.8 | 11 |
| 52 | 37.2 | 2.8 | 8 | 52 | 36.2 | 2.8 | 9 | 52 | 35.2 | 2.8 | 10 | 52 | 34.2 | 2.8 | 11 |
| 51 | 38.2 | 2.8 | 8 | 51 | 37.2 | 2.8 | 9 | 51 | 36.2 | 2.8 | 10 | 51 | 35.2 | 2.8 | 11 |
| 50 | 39.2 | 2.8 | 8 | 50 | 38.2 | 2.8 | 9 | 50 | 37.2 | 2.8 | 10 | 50 | 36.2 | 2.8 | 11 |
| 49 | 40.2 | 2.8 | 8 | 49 | 39.2 | 2.8 | 9 | 49 | 38.2 | 2.8 | 10 | 49 | 37.2 | 2.8 | 11 |
| 48 | 41.2 | 2.8 | 8 | 48 | 40.2 | 2.8 | 9 | 48 | 39.2 | 2.8 | 10 | 48 | 38.2 | 2.8 | 11 |
| 47 | 42.2 | 2.8 | 8 | 47 | 41.2 | 2.8 | 9 | 47 | 40.2 | 2.8 | 10 | 47 | 39.2 | 2.8 | 11 |
| 46 | 43.2 | 2.8 | 8 | 46 | 42.2 | 2.8 | 9 | 46 | 41.2 | 2.8 | 10 | 46 | 40.2 | 2.8 | 11 |
| 45 | 44.2 | 2.8 | 8 | 45 | 43.2 | 2.8 | 9 | 45 | 42.2 | 2.8 | 10 | 45 | 41.2 | 2.8 | 11 |
| 44 | 45.2 | 2.8 | 8 | 44 | 44.2 | 2.8 | 9 | 44 | 43.2 | 2.8 | 10 | 44 | 42.2 | 2.8 | 11 |
| 43 | 46.2 | 2.8 | 8 | 43 | 45.2 | 2.8 | 9 | 43 | 44.2 | 2.8 | 10 | 43 | 43.2 | 2.8 | 11 |
| 42 | 47.2 | 2.8 | 8 | 42 | 46.2 | 2.8 | 9 | 42 | 45.2 | 2.8 | 10 | 42 | 44.2 | 2.8 | 11 |
| 41 | 48.2 | 2.8 | 8 | 41 | 47.2 | 2.8 | 9 | 41 | 46.2 | 2.8 | 10 | 41 | 45.2 | 2.8 | 11 |
| 40 | 49.2 | 2.8 | 8 | 40 | 48.2 | 2.8 | 9 | 40 | 47.2 | 2.8 | 10 | 40 | 46.2 | 2.8 | 11 |
| 39 | 50.2 | 2.8 | 8 | 39 | 49.2 | 2.8 | 9 | 39 | 48.2 | 2.8 | 10 | 39 | 47.2 | 2.8 | 11 |
| 38 | 51.2 | 2.8 | 8 | 38 | 50.2 | 2.8 | 9 | 38 | 49.2 | 2.8 | 10 | 38 | 48.2 | 2.8 | 11 |
| 37 | 52.2 | 2.8 | 8 | 37 | 51.2 | 2.8 | 9 | 37 | 50.2 | 2.8 | 10 | 37 | 49.2 | 2.8 | 11 |
| 36 | 53.2 | 2.8 | 8 | 36 | 52.2 | 2.8 | 9 | 36 | 51.2 | 2.8 | 10 | 36 | 50.2 | 2.8 | 11 |
| 35 | 54.2 | 2.8 | 8 | 35 | 53.2 | 2.8 | 9 | 35 | 52.2 | 2.8 | 10 | 35 | 51.2 | 2.8 | 11 |
| 34 | 55.2 | 2.8 | 8 | 34 | 54.2 | 2.8 | 9 | 34 | 53.2 | 2.8 | 10 | 34 | 52.2 | 2.8 | 11 |
| 33 | 56.2 | 2.8 | 8 | 33 | 55.2 | 2.8 | 9 | 33 | 54.2 | 2.8 | 10 | 33 | 53.2 | 2.8 | 11 |
| 32 | 57.2 | 2.8 | 8 | 32 | 56.2 | 2.8 | 9 | 32 | 55.2 | 2.8 | 10 | 32 | 54.2 | 2.8 | 11 |
| 31 | 58.2 | 2.8 | 8 | 31 | 57.2 | 2.8 | 9 | 31 | 56.2 | 2.8 | 10 | 31 | 55.2 | 2.8 | 11 |
| 30 | 59.2 | 2.8 | 8 | 30 | 58.2 | 2.8 | 9 | 30 | 57.2 | 2.8 | 10 | 30 | 56.2 | 2.8 | 11 |
| 60 | 29 | 3 | 8 | 60 | 28 | 3 | 9 | 60 | 27 | 3 | 10 | 60 | 26 | 3 | 11 |
| 59 | 30 | 3 | 8 | 59 | 29 | 3 | 9 | 59 | 28 | 3 | 10 | 59 | 27 | 3 | 11 |
| 58 | 31 | 3 | 8 | 58 | 30 | 3 | 9 | 58 | 29 | 3 | 10 | 58 | 28 | 3 | 11 |
| 57 | 32 | 3 | 8 | 57 | 31 | 3 | 9 | 57 | 30 | 3 | 10 | 57 | 29 | 3 | 11 |
| 56 | 33 | 3 | 8 | 56 | 32 | 3 | 9 | 56 | 31 | 3 | 10 | 56 | 30 | 3 | 11 |
| 55 | 34 | 3 | 8 | 55 | 33 | 3 | 9 | 55 | 32 | 3 | 10 | 55 | 31 | 3 | 11 |
| 54 | 35 | 3 | 8 | 54 | 34 | 3 | 9 | 54 | 33 | 3 | 10 | 54 | 32 | 3 | 11 |
| 53 | 36 | 3 | 8 | 53 | 35 | 3 | 9 | 53 | 34 | 3 | 10 | 53 | 33 | 3 | 11 |
| 52 | 37 | 3 | 8 | 52 | 36 | 3 | 9 | 52 | 35 | 3 | 10 | 52 | 34 | 3 | 11 |
| 51 | 38 | 3 | 8 | 51 | 37 | 3 | 9 | 51 | 36 | 3 | 10 | 51 | 35 | 3 | 11 |
| 50 | 39 | 3 | 8 | 50 | 38 | 3 | 9 | 50 | 37 | 3 | 10 | 50 | 36 | 3 | 11 |
| 49 | 40 | 3 | 8 | 49 | 39 | 3 | 9 | 49 | 38 | 3 | 10 | 49 | 37 | 3 | 11 |
| 48 | 41 | 3 | 8 | 48 | 40 | 3 | 9 | 48 | 39 | 3 | 10 | 48 | 38 | 3 | 11 |
| 47 | 42 | 3 | 8 | 47 | 41 | 3 | 9 | 47 | 40 | 3 | 10 | 47 | 39 | 3 | 11 |
| 46 | 43 | 3 | 8 | 46 | 42 | 3 | 9 | 46 | 41 | 3 | 10 | 46 | 40 | 3 | 11 |
| 45 | 44 | 3 | 8 | 45 | 43 | 3 | 9 | 45 | 42 | 3 | 10 | 45 | 41 | 3 | 11 |
| 44 | 45 | 3 | 8 | 44 | 44 | 3 | 9 | 44 | 43 | 3 | 10 | 44 | 42 | 3 | 11 |
| 43 | 46 | 3 | 8 | 43 | 45 | 3 | 9 | 43 | 44 | 3 | 10 | 43 | 43 | 3 | 11 |
| 42 | 47 | 3 | 8 | 42 | 46 | 3 | 9 | 42 | 45 | 3 | 10 | 42 | 44 | 3 | 11 |
| 41 | 48 | 3 | 8 | 41 | 47 | 3 | 9 | 41 | 46 | 3 | 10 | 41 | 45 | 3 | 11 |
| 40 | 49 | 3 | 8 | 40 | 48 | 3 | 9 | 40 | 47 | 3 | 10 | 40 | 46 | 3 | 11 |
| 39 | 50 | 3 | 8 | 39 | 49 | 3 | 9 | 39 | 48 | 3 | 10 | 39 | 47 | 3 | 11 |
| 38 | 51 | 3 | 8 | 38 | 50 | 3 | 9 | 38 | 49 | 3 | 10 | 38 | 48 | 3 | 11 |
| 37 | 52 | 3 | 8 | 37 | 51 | 3 | 9 | 37 | 50 | 3 | 10 | 37 | 49 | 3 | 11 |
| 36 | 53 | 3 | 8 | 36 | 52 | 3 | 9 | 36 | 51 | 3 | 10 | 36 | 50 | 3 | 11 |
| 35 | 54 | 3 | 8 | 35 | 53 | 3 | 9 | 35 | 52 | 3 | 10 | 35 | 51 | 3 | 11 |
| 34 | 55 | 3 | 8 | 34 | 54 | 3 | 9 | 34 | 53 | 3 | 10 | 34 | 52 | 3 | 11 |
| 33 | 56 | 3 | 8 | 33 | 55 | 3 | 9 | 33 | 54 | 3 | 10 | 33 | 53 | 3 | 11 |
| 32 | 57 | 3 | 8 | 32 | 56 | 3 | 9 | 32 | 55 | 3 | 10 | 32 | 54 | 3 | 11 |
| 31 | 58 | 3 | 8 | 31 | 57 | 3 | 9 | 31 | 56 | 3 | 10 | 31 | 55 | 3 | 11 |
| 30 | 59 | 3 | 8 | 30 | 58 | 3 | 9 | 30 | 57 | 3 | 10 | 30 | 56 | 3 | 11 |
| 60 | 27 | 1 | 12 | 60 | 26 | 1 | 13 | 60 | 25 | 1 | 14 | 60 | 24 | 1 | 15 |
| 59 | 28 | 1 | 12 | 59 | 27 | 1 | 13 | 59 | 26 | 1 | 14 | 59 | 25 | 1 | 15 |
| 58 | 29 | 1 | 12 | 58 | 28 | 1 | 13 | 58 | 27 | 1 | 14 | 58 | 26 | 1 | 15 |
| 57 | 30 | 1 | 12 | 57 | 29 | 1 | 13 | 57 | 28 | 1 | 14 | 57 | 27 | 1 | 15 |
| 56 | 31 | 1 | 12 | 56 | 30 | 1 | 13 | 56 | 29 | 1 | 14 | 56 | 28 | 1 | 15 |
| 55 | 32 | 1 | 12 | 55 | 31 | 1 | 13 | 55 | 30 | 1 | 14 | 55 | 29 | 1 | 15 |
| 54 | 33 | 1 | 12 | 54 | 32 | 1 | 13 | 54 | 31 | 1 | 14 | 54 | 30 | 1 | 15 |
| 53 | 34 | 1 | 12 | 53 | 33 | 1 | 13 | 53 | 32 | 1 | 14 | 53 | 31 | 1 | 15 |
| 52 | 35 | 1 | 12 | 52 | 34 | 1 | 13 | 52 | 33 | 1 | 14 | 52 | 32 | 1 | 15 |
| 51 | 36 | 1 | 12 | 51 | 35 | 1 | 13 | 51 | 34 | 1 | 14 | 51 | 33 | 1 | 15 |
| 50 | 37 | 1 | 12 | 50 | 36 | 1 | 13 | 50 | 35 | 1 | 14 | 50 | 34 | 1 | 15 |
| 49 | 38 | 1 | 12 | 49 | 37 | 1 | 13 | 49 | 36 | 1 | 14 | 49 | 35 | 1 | 15 |
| 48 | 39 | 1 | 12 | 48 | 38 | 1 | 13 | 48 | 37 | 1 | 14 | 48 | 36 | 1 | 15 |
| 47 | 40 | 1 | 12 | 47 | 39 | 1 | 13 | 47 | 38 | 1 | 14 | 47 | 37 | 1 | 15 |
| 46 | 41 | 1 | 12 | 46 | 40 | 1 | 13 | 46 | 39 | 1 | 14 | 46 | 38 | 1 | 15 |
| 45 | 42 | 1 | 12 | 45 | 41 | 1 | 13 | 45 | 40 | 1 | 14 | 45 | 39 | 1 | 15 |
| 44 | 43 | 1 | 12 | 44 | 42 | 1 | 13 | 44 | 41 | 1 | 14 | 44 | 40 | 1 | 15 |
| 43 | 44 | 1 | 12 | 43 | 43 | 1 | 13 | 43 | 42 | 1 | 14 | 43 | 41 | 1 | 15 |
| 42 | 45 | 1 | 12 | 42 | 44 | 1 | 13 | 42 | 43 | 1 | 14 | 42 | 42 | 1 | 15 |
| 41 | 46 | 1 | 12 | 41 | 45 | 1 | 13 | 41 | 44 | 1 | 14 | 41 | 43 | 1 | 15 |
| 40 | 47 | 1 | 12 | 40 | 46 | 1 | 13 | 40 | 45 | 1 | 14 | 40 | 44 | 1 | 15 |
| 39 | 48 | 1 | 12 | 39 | 47 | 1 | 13 | 39 | 46 | 1 | 14 | 39 | 45 | 1 | 15 |
| 38 | 49 | 1 | 12 | 38 | 48 | 1 | 13 | 38 | 47 | 1 | 14 | 38 | 46 | 1 | 15 |
| 37 | 50 | 1 | 12 | 37 | 49 | 1 | 13 | 37 | 48 | 1 | 14 | 37 | 47 | 1 | 15 |
| 36 | 51 | 1 | 12 | 36 | 50 | 1 | 13 | 36 | 49 | 1 | 14 | 36 | 48 | 1 | 15 |
| 35 | 52 | 1 | 12 | 35 | 51 | 1 | 13 | 35 | 50 | 1 | 14 | 35 | 49 | 1 | 15 |
| 34 | 53 | 1 | 12 | 34 | 52 | 1 | 13 | 34 | 51 | 1 | 14 | 34 | 50 | 1 | 15 |
| 33 | 54 | 1 | 12 | 33 | 53 | 1 | 13 | 33 | 52 | 1 | 14 | 33 | 51 | 1 | 15 |
| 32 | 55 | 1 | 12 | 32 | 54 | 1 | 13 | 32 | 53 | 1 | 14 | 32 | 52 | 1 | 15 |
| 31 | 56 | 1 | 12 | 31 | 55 | 1 | 13 | 31 | 54 | 1 | 14 | 31 | 53 | 1 | 15 |
| 30 | 57 | 1 | 12 | 30 | 56 | 1 | 13 | 30 | 55 | 1 | 14 | 30 | 54 | 1 | 15 |
| 60 | 26.8 | 1.2 | 12 | 60 | 25.8 | 1.2 | 13 | 60 | 24.8 | 1.2 | 14 | 60 | 23.8 | 1.2 | 15 |
| 59 | 27.8 | 1.2 | 12 | 59 | 26.8 | 1.2 | 13 | 59 | 25.8 | 1.2 | 14 | 59 | 24.8 | 1.2 | 15 |
| 58 | 28.8 | 1.2 | 12 | 58 | 27.8 | 1.2 | 13 | 58 | 26.8 | 1.2 | 14 | 58 | 25.8 | 1.2 | 15 |
| 57 | 29.8 | 1.2 | 12 | 57 | 28.8 | 1.2 | 13 | 57 | 27.8 | 1.2 | 14 | 57 | 26.8 | 1.2 | 15 |
| 56 | 30.8 | 1.2 | 12 | 56 | 29.8 | 1.2 | 13 | 56 | 28.8 | 1.2 | 14 | 56 | 27.8 | 1.2 | 15 |
| 55 | 31.8 | 1.2 | 12 | 55 | 30.8 | 1.2 | 13 | 55 | 29.8 | 1.2 | 14 | 55 | 28.8 | 1.2 | 15 |
| 54 | 32.8 | 1.2 | 12 | 54 | 31.8 | 1.2 | 13 | 54 | 30.8 | 1.2 | 14 | 54 | 29.8 | 1.2 | 15 |
| 53 | 33.8 | 1.2 | 12 | 53 | 32.8 | 1.2 | 13 | 53 | 31.8 | 1.2 | 14 | 53 | 30.8 | 1.2 | 15 |
| 52 | 34.8 | 1.2 | 12 | 52 | 33.8 | 1.2 | 13 | 52 | 32.8 | 1.2 | 14 | 52 | 31.8 | 1.2 | 15 |
| 51 | 35.8 | 1.2 | 12 | 51 | 34.8 | 1.2 | 13 | 51 | 33.8 | 1.2 | 14 | 51 | 32.8 | 1.2 | 15 |
| 50 | 36.8 | 1.2 | 12 | 50 | 35.8 | 1.2 | 13 | 50 | 34.8 | 1.2 | 14 | 50 | 33.8 | 1.2 | 15 |
| 49 | 37.8 | 1.2 | 12 | 49 | 36.8 | 1.2 | 13 | 49 | 35.8 | 1.2 | 14 | 49 | 34.8 | 1.2 | 15 |
| 48 | 38.8 | 1.2 | 12 | 48 | 37.8 | 1.2 | 13 | 48 | 36.8 | 1.2 | 14 | 48 | 35.8 | 1.2 | 15 |
| 47 | 39.8 | 1.2 | 12 | 47 | 38.8 | 1.2 | 13 | 47 | 37.8 | 1.2 | 14 | 47 | 36.8 | 1.2 | 15 |
| 46 | 40.8 | 1.2 | 12 | 46 | 39.8 | 1.2 | 13 | 46 | 38.8 | 1.2 | 14 | 46 | 37.8 | 1.2 | 15 |
| 45 | 41.8 | 1.2 | 12 | 45 | 40.8 | 1.2 | 13 | 45 | 39.8 | 1.2 | 14 | 45 | 38.8 | 1.2 | 15 |
| 44 | 42.8 | 1.2 | 12 | 44 | 41.8 | 1.2 | 13 | 44 | 40.8 | 1.2 | 14 | 44 | 39.8 | 1.2 | 15 |
| 43 | 43.8 | 1.2 | 12 | 43 | 42.8 | 1.2 | 13 | 43 | 41.8 | 1.2 | 14 | 43 | 40.8 | 1.2 | 15 |
| 42 | 44.8 | 1.2 | 12 | 42 | 43.8 | 1.2 | 13 | 42 | 42.8 | 1.2 | 14 | 42 | 41.8 | 1.2 | 15 |
| 41 | 45.8 | 1.2 | 12 | 41 | 44.8 | 1.2 | 13 | 41 | 43.8 | 1.2 | 14 | 41 | 42.8 | 1.2 | 15 |
| 40 | 46.8 | 1.2 | 12 | 40 | 45.8 | 1.2 | 13 | 40 | 44.8 | 1.2 | 14 | 40 | 43.8 | 1.2 | 15 |
| 39 | 47.8 | 1.2 | 12 | 39 | 46.8 | 1.2 | 13 | 39 | 45.8 | 1.2 | 14 | 39 | 44.8 | 1.2 | 15 |
| 38 | 48.8 | 1.2 | 12 | 38 | 47.8 | 1.2 | 13 | 38 | 46.8 | 1.2 | 14 | 38 | 45.8 | 1.2 | 15 |
| 37 | 49.8 | 1.2 | 12 | 37 | 48.8 | 1.2 | 13 | 37 | 47.8 | 1.2 | 14 | 37 | 46.8 | 1.2 | 15 |
| 36 | 50.8 | 1.2 | 12 | 36 | 49.8 | 1.2 | 13 | 36 | 48.8 | 1.2 | 14 | 36 | 47.8 | 1.2 | 15 |
| 35 | 51.8 | 1.2 | 12 | 35 | 50.8 | 1.2 | 13 | 35 | 49.8 | 1.2 | 14 | 35 | 48.8 | 1.2 | 15 |
| 34 | 52.8 | 1.2 | 12 | 34 | 51.8 | 1.2 | 13 | 34 | 50.8 | 1.2 | 14 | 34 | 49.8 | 1.2 | 15 |
| 33 | 53.8 | 1.2 | 12 | 33 | 52.8 | 1.2 | 13 | 33 | 51.8 | 1.2 | 14 | 33 | 50.8 | 1.2 | 15 |
| 32 | 54.8 | 1.2 | 12 | 32 | 53.8 | 1.2 | 13 | 32 | 52.8 | 1.2 | 14 | 32 | 51.8 | 1.2 | 15 |
| 31 | 55.8 | 1.2 | 12 | 31 | 54.8 | 1.2 | 13 | 31 | 53.8 | 1.2 | 14 | 31 | 52.8 | 1.2 | 15 |
| 30 | 56.8 | 1.2 | 12 | 30 | 55.8 | 1.2 | 13 | 30 | 54.8 | 1.2 | 14 | 30 | 53.8 | 1.2 | 15 |
| 60 | 26.6 | 1.4 | 12 | 60 | 25.6 | 1.4 | 13 | 60 | 24.6 | 1.4 | 14 | 60 | 23.6 | 1.4 | 15 |
| 59 | 27.6 | 1.4 | 12 | 59 | 26.6 | 1.4 | 13 | 59 | 25.6 | 1.4 | 14 | 59 | 24.6 | 1.4 | 15 |
| 58 | 28.6 | 1.4 | 12 | 58 | 27.6 | 1.4 | 13 | 58 | 26.6 | 1.4 | 14 | 58 | 25.6 | 1.4 | 15 |
| 57 | 29.6 | 1.4 | 12 | 57 | 28.6 | 1.4 | 13 | 57 | 27.6 | 1.4 | 14 | 57 | 26.6 | 1.4 | 15 |
| 56 | 30.6 | 1.4 | 12 | 56 | 29.6 | 1.4 | 13 | 56 | 28.6 | 1.4 | 14 | 56 | 27.6 | 1.4 | 15 |
| 55 | 31.6 | 1.4 | 12 | 55 | 30.6 | 1.4 | 13 | 55 | 29.6 | 1.4 | 14 | 55 | 28.6 | 1.4 | 15 |
| 54 | 32.6 | 1.4 | 12 | 54 | 31.6 | 1.4 | 13 | 54 | 30.6 | 1.4 | 14 | 54 | 29.6 | 1.4 | 15 |
| 53 | 33.6 | 1.4 | 12 | 53 | 32.6 | 1.4 | 13 | 53 | 31.6 | 1.4 | 14 | 53 | 30.6 | 1.4 | 15 |
| 52 | 34.6 | 1.4 | 12 | 52 | 33.6 | 1.4 | 13 | 52 | 32.6 | 1.4 | 14 | 52 | 31.6 | 1.4 | 15 |
| 51 | 35.6 | 1.4 | 12 | 51 | 34.6 | 1.4 | 13 | 51 | 33.6 | 1.4 | 14 | 51 | 32.6 | 1.4 | 15 |
| 50 | 36.6 | 1.4 | 12 | 50 | 35.6 | 1.4 | 13 | 50 | 34.6 | 1.4 | 14 | 50 | 33.6 | 1.4 | 15 |
| 49 | 37.6 | 1.4 | 12 | 49 | 36.6 | 1.4 | 13 | 49 | 35.6 | 1.4 | 14 | 49 | 34.6 | 1.4 | 15 |
| 48 | 38.6 | 1.4 | 12 | 48 | 37.6 | 1.4 | 13 | 48 | 36.6 | 1.4 | 14 | 48 | 35.6 | 1.4 | 15 |
| 47 | 39.6 | 1.4 | 12 | 47 | 38.6 | 1.4 | 13 | 47 | 37.6 | 1.4 | 14 | 47 | 36.6 | 1.4 | 15 |
| 46 | 40.6 | 1.4 | 12 | 46 | 39.6 | 1.4 | 13 | 46 | 38.6 | 1.4 | 14 | 46 | 37.6 | 1.4 | 15 |
| 45 | 41.6 | 1.4 | 12 | 45 | 40.6 | 1.4 | 13 | 45 | 39.6 | 1.4 | 14 | 45 | 38.6 | 1.4 | 15 |
| 44 | 42.6 | 1.4 | 12 | 44 | 41.6 | 1.4 | 13 | 44 | 40.6 | 1.4 | 14 | 44 | 39.6 | 1.4 | 15 |
| 43 | 43.6 | 1.4 | 12 | 43 | 42.6 | 1.4 | 13 | 43 | 41.6 | 1.4 | 14 | 43 | 40.6 | 1.4 | 15 |
| 42 | 44.6 | 1.4 | 12 | 42 | 43.6 | 1.4 | 13 | 42 | 42.6 | 1.4 | 14 | 42 | 41.6 | 1.4 | 15 |
| 41 | 45.6 | 1.4 | 12 | 41 | 44.6 | 1.4 | 13 | 41 | 43.6 | 1.4 | 14 | 41 | 42.6 | 1.4 | 15 |
| 40 | 46.6 | 1.4 | 12 | 40 | 45.6 | 1.4 | 13 | 40 | 44.6 | 1.4 | 14 | 40 | 43.6 | 1.4 | 15 |
| 39 | 47.6 | 1.4 | 12 | 39 | 46.6 | 1.4 | 13 | 39 | 45.6 | 1.4 | 14 | 39 | 44.6 | 1.4 | 15 |
| 38 | 48.6 | 1.4 | 12 | 38 | 47.6 | 1.4 | 13 | 38 | 46.6 | 1.4 | 14 | 38 | 45.6 | 1.4 | 15 |
| 37 | 49.6 | 1.4 | 12 | 37 | 48.6 | 1.4 | 13 | 37 | 47.6 | 1.4 | 14 | 37 | 46.6 | 1.4 | 15 |
| 36 | 50.6 | 1.4 | 12 | 36 | 49.6 | 1.4 | 13 | 36 | 48.6 | 1.4 | 14 | 36 | 47.6 | 1.4 | 15 |
| 35 | 51.6 | 1.4 | 12 | 35 | 50.6 | 1.4 | 13 | 35 | 49.6 | 1.4 | 14 | 35 | 48.6 | 1.4 | 15 |
| 34 | 52.6 | 1.4 | 12 | 34 | 51.6 | 1.4 | 13 | 34 | 50.6 | 1.4 | 14 | 34 | 49.6 | 1.4 | 15 |
| 33 | 53.6 | 1.4 | 12 | 33 | 52.6 | 1.4 | 13 | 33 | 51.6 | 1.4 | 14 | 33 | 50.6 | 1.4 | 15 |
| 32 | 54.6 | 1.4 | 12 | 32 | 53.6 | 1.4 | 13 | 32 | 52.6 | 1.4 | 14 | 32 | 51.6 | 1.4 | 15 |
| 31 | 55.6 | 1.4 | 12 | 31 | 54.6 | 1.4 | 13 | 31 | 53.6 | 1.4 | 14 | 31 | 52.6 | 1.4 | 15 |
| 30 | 56.6 | 1.4 | 12 | 30 | 55.6 | 1.4 | 13 | 30 | 54.6 | 1.4 | 14 | 30 | 53.6 | 1.4 | 15 |
| 60 | 26.4 | 1.6 | 12 | 60 | 25.4 | 1.6 | 13 | 60 | 24.4 | 1.6 | 14 | 60 | 23.4 | 1.6 | 15 |
| 59 | 27.4 | 1.6 | 12 | 59 | 26.4 | 1.6 | 13 | 59 | 25.4 | 1.6 | 14 | 59 | 24.4 | 1.6 | 15 |
| 58 | 28.4 | 1.6 | 12 | 58 | 27.4 | 1.6 | 13 | 58 | 26.4 | 1.6 | 14 | 58 | 25.4 | 1.6 | 15 |
| 57 | 29.4 | 1.6 | 12 | 57 | 28.4 | 1.6 | 13 | 57 | 27.4 | 1.6 | 14 | 57 | 26.4 | 1.6 | 15 |
| 56 | 30.4 | 1.6 | 12 | 56 | 29.4 | 1.6 | 13 | 56 | 28.4 | 1.6 | 14 | 56 | 27.4 | 1.6 | 15 |
| 55 | 31.4 | 1.6 | 12 | 55 | 30.4 | 1.6 | 13 | 55 | 29.4 | 1.6 | 14 | 55 | 28.4 | 1.6 | 15 |
| 54 | 32.4 | 1.6 | 12 | 54 | 31.4 | 1.6 | 13 | 54 | 30.4 | 1.6 | 14 | 54 | 29.4 | 1.6 | 15 |
| 53 | 33.4 | 1.6 | 12 | 53 | 32.4 | 1.6 | 13 | 53 | 31.4 | 1.6 | 14 | 53 | 30.4 | 1.6 | 15 |
| 52 | 34.4 | 1.6 | 12 | 52 | 33.4 | 1.6 | 13 | 52 | 32.4 | 1.6 | 14 | 52 | 31.4 | 1.6 | 15 |
| 51 | 35.4 | 1.6 | 12 | 51 | 34.4 | 1.6 | 13 | 51 | 33.4 | 1.6 | 14 | 51 | 32.4 | 1.6 | 15 |
| 50 | 36.4 | 1.6 | 12 | 50 | 35.4 | 1.6 | 13 | 50 | 34.4 | 1.6 | 14 | 50 | 33.4 | 1.6 | 15 |
| 49 | 37.4 | 1.6 | 12 | 49 | 36.4 | 1.6 | 13 | 49 | 35.4 | 1.6 | 14 | 49 | 34.4 | 1.6 | 15 |
| 48 | 38.4 | 1.6 | 12 | 48 | 37.4 | 1.6 | 13 | 48 | 36.4 | 1.6 | 14 | 48 | 35.4 | 1.6 | 15 |
| 47 | 39.4 | 1.6 | 12 | 47 | 38.4 | 1.6 | 13 | 47 | 37.4 | 1.6 | 14 | 47 | 36.4 | 1.6 | 15 |
| 46 | 40.4 | 1.6 | 12 | 46 | 39.4 | 1.6 | 13 | 46 | 38.4 | 1.6 | 14 | 46 | 37.4 | 1.6 | 15 |
| 45 | 41.4 | 1.6 | 12 | 45 | 40.4 | 1.6 | 13 | 45 | 39.4 | 1.6 | 14 | 45 | 38.4 | 1.6 | 15 |
| 44 | 42.4 | 1.6 | 12 | 44 | 41.4 | 1.6 | 13 | 44 | 40.4 | 1.6 | 14 | 44 | 39.4 | 1.6 | 15 |
| 43 | 43.4 | 1.6 | 12 | 43 | 42.4 | 1.6 | 13 | 43 | 41.4 | 1.6 | 14 | 43 | 40.4 | 1.6 | 15 |
| 42 | 44.4 | 1.6 | 12 | 42 | 43.4 | 1.6 | 13 | 42 | 42.4 | 1.6 | 14 | 42 | 41.4 | 1.6 | 15 |
| 41 | 45.4 | 1.6 | 12 | 41 | 44.4 | 1.6 | 13 | 41 | 43.4 | 1.6 | 14 | 41 | 42.4 | 1.6 | 15 |
| 40 | 46.4 | 1.6 | 12 | 40 | 45.4 | 1.6 | 13 | 40 | 44.4 | 1.6 | 14 | 40 | 43.4 | 1.6 | 15 |
| 39 | 47.4 | 1.6 | 12 | 39 | 46.4 | 1.6 | 13 | 39 | 45.4 | 1.6 | 14 | 39 | 44.4 | 1.6 | 15 |
| 38 | 48.4 | 1.6 | 12 | 38 | 47.4 | 1.6 | 13 | 38 | 46.4 | 1.6 | 14 | 38 | 45.4 | 1.6 | 15 |
| 37 | 49.4 | 1.6 | 12 | 37 | 48.4 | 1.6 | 13 | 37 | 47.4 | 1.6 | 14 | 37 | 46.4 | 1.6 | 15 |
| 36 | 50.4 | 1.6 | 12 | 36 | 49.4 | 1.6 | 13 | 36 | 48.4 | 1.6 | 14 | 36 | 47.4 | 1.6 | 15 |
| 35 | 51.4 | 1.6 | 12 | 35 | 50.4 | 1.6 | 13 | 35 | 49.4 | 1.6 | 14 | 35 | 48.4 | 1.6 | 15 |
| 34 | 52.4 | 1.6 | 12 | 34 | 51.4 | 1.6 | 13 | 34 | 50.4 | 1.6 | 14 | 34 | 49.4 | 1.6 | 15 |
| 33 | 53.4 | 1.6 | 12 | 33 | 52.4 | 1.6 | 13 | 33 | 51.4 | 1.6 | 14 | 33 | 50.4 | 1.6 | 15 |
| 32 | 54.4 | 1.6 | 12 | 32 | 53.4 | 1.6 | 13 | 32 | 52.4 | 1.6 | 14 | 32 | 51.4 | 1.6 | 15 |
| 31 | 55.4 | 1.6 | 12 | 31 | 54.4 | 1.6 | 13 | 31 | 53.4 | 1.6 | 14 | 31 | 52.4 | 1.6 | 15 |
| 30 | 56.4 | 1.6 | 12 | 30 | 55.4 | 1.6 | 13 | 30 | 54.4 | 1.6 | 14 | 30 | 53.4 | 1.6 | 15 |
| 60 | 26.2 | 1.8 | 12 | 60 | 25.2 | 1.8 | 13 | 60 | 24.2 | 1.8 | 14 | 60 | 23.2 | 1.8 | 15 |
| 59 | 27.2 | 1.8 | 12 | 59 | 26.2 | 1.8 | 13 | 59 | 25.2 | 1.8 | 14 | 59 | 24.2 | 1.8 | 15 |
| 58 | 28.2 | 1.8 | 12 | 58 | 27.2 | 1.8 | 13 | 58 | 26.2 | 1.8 | 14 | 58 | 25.2 | 1.8 | 15 |
| 57 | 29.2 | 1.8 | 12 | 57 | 28.2 | 1.8 | 13 | 57 | 27.2 | 1.8 | 14 | 57 | 26.2 | 1.8 | 15 |
| 56 | 30.2 | 1.8 | 12 | 56 | 29.2 | 1.8 | 13 | 56 | 28.2 | 1.8 | 14 | 56 | 27.2 | 1.8 | 15 |
| 55 | 31.2 | 1.8 | 12 | 55 | 30.2 | 1.8 | 13 | 55 | 29.2 | 1.8 | 14 | 55 | 28.2 | 1.8 | 15 |
| 54 | 32.2 | 1.8 | 12 | 54 | 31.2 | 1.8 | 13 | 54 | 30.2 | 1.8 | 14 | 54 | 29.2 | 1.8 | 15 |
| 53 | 33.2 | 1.8 | 12 | 53 | 32.2 | 1.8 | 13 | 53 | 31.2 | 1.8 | 14 | 53 | 30.2 | 1.8 | 15 |
| 52 | 34.2 | 1.8 | 12 | 52 | 33.2 | 1.8 | 13 | 52 | 32.2 | 1.8 | 14 | 52 | 31.2 | 1.8 | 15 |
| 51 | 35.2 | 1.8 | 12 | 51 | 34.2 | 1.8 | 13 | 51 | 33.2 | 1.8 | 14 | 51 | 32.2 | 1.8 | 15 |
| 50 | 36.2 | 1.8 | 12 | 50 | 35.2 | 1.8 | 13 | 50 | 34.2 | 1.8 | 14 | 50 | 33.2 | 1.8 | 15 |
| 49 | 37.2 | 1.8 | 12 | 49 | 36.2 | 1.8 | 13 | 49 | 35.2 | 1.8 | 14 | 49 | 34.2 | 1.8 | 15 |
| 48 | 38.2 | 1.8 | 12 | 48 | 37.2 | 1.8 | 13 | 48 | 36.2 | 1.8 | 14 | 48 | 35.2 | 1.8 | 15 |
| 47 | 39.2 | 1.8 | 12 | 47 | 38.2 | 1.8 | 13 | 47 | 37.2 | 1.8 | 14 | 47 | 36.2 | 1.8 | 15 |
| 46 | 40.2 | 1.8 | 12 | 46 | 39.2 | 1.8 | 13 | 46 | 38.2 | 1.8 | 14 | 46 | 37.2 | 1.8 | 15 |
| 45 | 41.2 | 1.8 | 12 | 45 | 40.2 | 1.8 | 13 | 45 | 39.2 | 1.8 | 14 | 45 | 38.2 | 1.8 | 15 |
| 44 | 42.2 | 1.8 | 12 | 44 | 41.2 | 1.8 | 13 | 44 | 40.2 | 1.8 | 14 | 44 | 39.2 | 1.8 | 15 |
| 43 | 43.2 | 1.8 | 12 | 43 | 42.2 | 1.8 | 13 | 43 | 41.2 | 1.8 | 14 | 43 | 40.2 | 1.8 | 15 |
| 42 | 44.2 | 1.8 | 12 | 42 | 43.2 | 1.8 | 13 | 42 | 42.2 | 1.8 | 14 | 42 | 41.2 | 1.8 | 15 |
| 41 | 45.2 | 1.8 | 12 | 41 | 44.2 | 1.8 | 13 | 41 | 43.2 | 1.8 | 14 | 41 | 42.2 | 1.8 | 15 |
| 40 | 46.2 | 1.8 | 12 | 40 | 45.2 | 1.8 | 13 | 40 | 44.2 | 1.8 | 14 | 40 | 43.2 | 1.8 | 15 |
| 39 | 47.2 | 1.8 | 12 | 39 | 46.2 | 1.8 | 13 | 39 | 45.2 | 1.8 | 14 | 39 | 44.2 | 1.8 | 15 |
| 38 | 48.2 | 1.8 | 12 | 38 | 47.2 | 1.8 | 13 | 38 | 46.2 | 1.8 | 14 | 38 | 45.2 | 1.8 | 15 |
| 37 | 49.2 | 1.8 | 12 | 37 | 48.2 | 1.8 | 13 | 37 | 47.2 | 1.8 | 14 | 37 | 46.2 | 1.8 | 15 |
| 36 | 50.2 | 1.8 | 12 | 36 | 49.2 | 1.8 | 13 | 36 | 48.2 | 1.8 | 14 | 36 | 47.2 | 1.8 | 15 |
| 35 | 51.2 | 1.8 | 12 | 35 | 50.2 | 1.8 | 13 | 35 | 49.2 | 1.8 | 14 | 35 | 48.2 | 1.8 | 15 |
| 34 | 52.2 | 1.8 | 12 | 34 | 51.2 | 1.8 | 13 | 34 | 50.2 | 1.8 | 14 | 34 | 49.2 | 1.8 | 15 |
| 33 | 53.2 | 1.8 | 12 | 33 | 52.2 | 1.8 | 13 | 33 | 51.2 | 1.8 | 14 | 33 | 50.2 | 1.8 | 15 |
| 32 | 54.2 | 1.8 | 12 | 32 | 53.2 | 1.8 | 13 | 32 | 52.2 | 1.8 | 14 | 32 | 51.2 | 1.8 | 15 |
| 31 | 55.2 | 1.8 | 12 | 31 | 54.2 | 1.8 | 13 | 31 | 53.2 | 1.8 | 14 | 31 | 52.2 | 1.8 | 15 |
| 30 | 56.2 | 1.8 | 12 | 30 | 55.2 | 1.8 | 13 | 30 | 54.2 | 1.8 | 14 | 30 | 53.2 | 1.8 | 15 |
| 60 | 26 | 2 | 12 | 60 | 25 | 2 | 13 | 60 | 24 | 2 | 14 | 60 | 23 | 2 | 15 |
| 59 | 27 | 2 | 12 | 59 | 26 | 2 | 13 | 59 | 25 | 2 | 14 | 59 | 24 | 2 | 15 |
| 58 | 28 | 2 | 12 | 58 | 27 | 2 | 13 | 58 | 26 | 2 | 14 | 58 | 25 | 2 | 15 |
| 57 | 29 | 2 | 12 | 57 | 28 | 2 | 13 | 57 | 27 | 2 | 14 | 57 | 26 | 2 | 15 |
| 56 | 30 | 2 | 12 | 56 | 29 | 2 | 13 | 56 | 28 | 2 | 14 | 56 | 27 | 2 | 15 |
| 55 | 31 | 2 | 12 | 55 | 30 | 2 | 13 | 55 | 29 | 2 | 14 | 55 | 28 | 2 | 15 |
| 54 | 32 | 2 | 12 | 54 | 31 | 2 | 13 | 54 | 30 | 2 | 14 | 54 | 29 | 2 | 15 |
| 53 | 33 | 2 | 12 | 53 | 32 | 2 | 13 | 53 | 31 | 2 | 14 | 53 | 30 | 2 | 15 |
| 52 | 34 | 2 | 12 | 52 | 33 | 2 | 13 | 52 | 32 | 2 | 14 | 52 | 31 | 2 | 15 |
| 51 | 35 | 2 | 12 | 51 | 34 | 2 | 13 | 51 | 33 | 2 | 14 | 51 | 32 | 2 | 15 |
| 50 | 36 | 2 | 12 | 50 | 35 | 2 | 13 | 50 | 34 | 2 | 14 | 50 | 33 | 2 | 15 |
| 49 | 37 | 2 | 12 | 49 | 36 | 2 | 13 | 49 | 35 | 2 | 14 | 49 | 34 | 2 | 15 |
| 48 | 38 | 2 | 12 | 48 | 37 | 2 | 13 | 48 | 36 | 2 | 14 | 48 | 35 | 2 | 15 |
| 47 | 39 | 2 | 12 | 47 | 38 | 2 | 13 | 47 | 37 | 2 | 14 | 47 | 36 | 2 | 15 |
| 46 | 40 | 2 | 12 | 46 | 39 | 2 | 13 | 46 | 38 | 2 | 14 | 46 | 37 | 2 | 15 |
| 45 | 41 | 2 | 12 | 45 | 40 | 2 | 13 | 45 | 39 | 2 | 14 | 45 | 38 | 2 | 15 |
| 44 | 42 | 2 | 12 | 44 | 41 | 2 | 13 | 44 | 40 | 2 | 14 | 44 | 39 | 2 | 15 |
| 43 | 43 | 2 | 12 | 43 | 42 | 2 | 13 | 43 | 41 | 2 | 14 | 43 | 40 | 2 | 15 |
| 42 | 44 | 2 | 12 | 42 | 43 | 2 | 13 | 42 | 42 | 2 | 14 | 42 | 41 | 2 | 15 |
| 41 | 45 | 2 | 12 | 41 | 44 | 2 | 13 | 41 | 43 | 2 | 14 | 41 | 42 | 2 | 15 |
| 40 | 46 | 2 | 12 | 40 | 45 | 2 | 13 | 40 | 44 | 2 | 14 | 40 | 43 | 2 | 15 |
| 39 | 47 | 2 | 12 | 39 | 46 | 2 | 13 | 39 | 45 | 2 | 14 | 39 | 44 | 2 | 15 |
| 38 | 48 | 2 | 12 | 38 | 47 | 2 | 13 | 38 | 46 | 2 | 14 | 38 | 45 | 2 | 15 |
| 37 | 49 | 2 | 12 | 37 | 48 | 2 | 13 | 37 | 47 | 2 | 14 | 37 | 46 | 2 | 15 |
| 36 | 50 | 2 | 12 | 36 | 49 | 2 | 13 | 36 | 48 | 2 | 14 | 36 | 47 | 2 | 15 |
| 35 | 51 | 2 | 12 | 35 | 50 | 2 | 13 | 35 | 49 | 2 | 14 | 35 | 48 | 2 | 15 |
| 34 | 52 | 2 | 12 | 34 | 51 | 2 | 13 | 34 | 50 | 2 | 14 | 34 | 49 | 2 | 15 |
| 33 | 53 | 2 | 12 | 33 | 52 | 2 | 13 | 33 | 51 | 2 | 14 | 33 | 50 | 2 | 15 |
| 32 | 54 | 2 | 12 | 32 | 53 | 2 | 13 | 32 | 52 | 2 | 14 | 32 | 51 | 2 | 15 |
| 31 | 55 | 2 | 12 | 31 | 54 | 2 | 13 | 31 | 53 | 2 | 14 | 31 | 52 | 2 | 15 |
| 30 | 56 | 2 | 12 | 30 | 55 | 2 | 13 | 30 | 54 | 2 | 14 | 30 | 53 | 2 | 15 |
| 60 | 25.8 | 2.2 | 12 | 60 | 24.8 | 2.2 | 13 | 60 | 23.8 | 2.2 | 14 | 60 | 22.8 | 2.2 | 15 |
| 59 | 26.8 | 2.2 | 12 | 59 | 25.8 | 2.2 | 13 | 59 | 24.8 | 2.2 | 14 | 59 | 23.8 | 2.2 | 15 |
| 58 | 27.8 | 2.2 | 12 | 58 | 26.8 | 2.2 | 13 | 58 | 25.8 | 2.2 | 14 | 58 | 24.8 | 2.2 | 15 |
| 57 | 28.8 | 2.2 | 12 | 57 | 27.8 | 2.2 | 13 | 57 | 26.8 | 2.2 | 14 | 57 | 25.8 | 2.2 | 15 |
| 56 | 29.8 | 2.2 | 12 | 56 | 28.8 | 2.2 | 13 | 56 | 27.8 | 2.2 | 14 | 56 | 26.8 | 2.2 | 15 |
| 55 | 30.8 | 2.2 | 12 | 55 | 29.8 | 2.2 | 13 | 55 | 28.8 | 2.2 | 14 | 55 | 27.8 | 2.2 | 15 |
| 54 | 31.8 | 2.2 | 12 | 54 | 30.8 | 2.2 | 13 | 54 | 29.8 | 2.2 | 14 | 54 | 28.8 | 2.2 | 15 |
| 53 | 32.8 | 2.2 | 12 | 53 | 31.8 | 2.2 | 13 | 53 | 30.8 | 2.2 | 14 | 53 | 29.8 | 2.2 | 15 |
| 52 | 33.8 | 2.2 | 12 | 52 | 32.8 | 2.2 | 13 | 52 | 31.8 | 2.2 | 14 | 52 | 30.8 | 2.2 | 15 |
| 51 | 34.8 | 2.2 | 12 | 51 | 33.8 | 2.2 | 13 | 51 | 32.8 | 2.2 | 14 | 51 | 31.8 | 2.2 | 15 |
| 50 | 35.8 | 2.2 | 12 | 50 | 34.8 | 2.2 | 13 | 50 | 33.8 | 2.2 | 14 | 50 | 32.8 | 2.2 | 15 |
| 49 | 36.8 | 2.2 | 12 | 49 | 35.8 | 2.2 | 13 | 49 | 34.8 | 2.2 | 14 | 49 | 33.8 | 2.2 | 15 |
| 48 | 37.8 | 2.2 | 12 | 48 | 36.8 | 2.2 | 13 | 48 | 35.8 | 2.2 | 14 | 48 | 34.8 | 2.2 | 15 |
| 47 | 38.8 | 2.2 | 12 | 47 | 37.8 | 2.2 | 13 | 47 | 36.8 | 2.2 | 14 | 47 | 35.8 | 2.2 | 15 |
| 46 | 39.8 | 2.2 | 12 | 46 | 38.8 | 2.2 | 13 | 46 | 37.8 | 2.2 | 14 | 46 | 36.8 | 2.2 | 15 |
| 45 | 40.8 | 2.2 | 12 | 45 | 39.8 | 2.2 | 13 | 45 | 38.8 | 2.2 | 14 | 45 | 37.8 | 2.2 | 15 |
| 44 | 41.8 | 2.2 | 12 | 44 | 40.8 | 2.2 | 13 | 44 | 39.8 | 2.2 | 14 | 44 | 38.8 | 2.2 | 15 |
| 43 | 42.8 | 2.2 | 12 | 43 | 41.8 | 2.2 | 13 | 43 | 40.8 | 2.2 | 14 | 43 | 39.8 | 2.2 | 15 |
| 42 | 43.8 | 2.2 | 12 | 42 | 42.8 | 2.2 | 13 | 42 | 41.8 | 2.2 | 14 | 42 | 40.8 | 2.2 | 15 |
| 41 | 44.8 | 2.2 | 12 | 41 | 43.8 | 2.2 | 13 | 41 | 42.8 | 2.2 | 14 | 41 | 41.8 | 2.2 | 15 |
| 40 | 45.8 | 2.2 | 12 | 40 | 44.8 | 2.2 | 13 | 40 | 43.8 | 2.2 | 14 | 40 | 42.8 | 2.2 | 15 |
| 39 | 46.8 | 2.2 | 12 | 39 | 45.8 | 2.2 | 13 | 39 | 44.8 | 2.2 | 14 | 39 | 43.8 | 2.2 | 15 |
| 38 | 47.8 | 2.2 | 12 | 38 | 46.8 | 2.2 | 13 | 38 | 45.8 | 2.2 | 14 | 38 | 44.8 | 2.2 | 15 |
| 37 | 48.8 | 2.2 | 12 | 37 | 47.8 | 2.2 | 13 | 37 | 46.8 | 2.2 | 14 | 37 | 45.8 | 2.2 | 15 |
| 36 | 49.8 | 2.2 | 12 | 36 | 48.8 | 2.2 | 13 | 36 | 47.8 | 2.2 | 14 | 36 | 46.8 | 2.2 | 15 |
| 35 | 50.8 | 2.2 | 12 | 35 | 49.8 | 2.2 | 13 | 35 | 48.8 | 2.2 | 14 | 35 | 47.8 | 2.2 | 15 |
| 34 | 51.8 | 2.2 | 12 | 34 | 50.8 | 2.2 | 13 | 34 | 49.8 | 2.2 | 14 | 34 | 48.8 | 2.2 | 15 |
| 33 | 52.8 | 2.2 | 12 | 33 | 51.8 | 2.2 | 13 | 33 | 50.8 | 2.2 | 14 | 33 | 49.8 | 2.2 | 15 |
| 32 | 53.8 | 2.2 | 12 | 32 | 52.8 | 2.2 | 13 | 32 | 51.8 | 2.2 | 14 | 32 | 50.8 | 2.2 | 15 |
| 31 | 54.8 | 2.2 | 12 | 31 | 53.8 | 2.2 | 13 | 31 | 52.8 | 2.2 | 14 | 31 | 51.8 | 2.2 | 15 |
| 30 | 55.8 | 2.2 | 12 | 30 | 54.8 | 2.2 | 13 | 30 | 53.8 | 2.2 | 14 | 30 | 52.8 | 2.2 | 15 |
| 60 | 25.6 | 2.4 | 12 | 60 | 24.6 | 2.4 | 13 | 60 | 23.6 | 2.4 | 14 | 60 | 22.6 | 2.4 | 15 |
| 59 | 26.6 | 2.4 | 12 | 59 | 25.6 | 2.4 | 13 | 59 | 24.6 | 2.4 | 14 | 59 | 23.6 | 2.4 | 15 |
| 58 | 27.6 | 2.4 | 12 | 58 | 26.6 | 2.4 | 13 | 58 | 25.6 | 2.4 | 14 | 58 | 24.6 | 2.4 | 15 |
| 57 | 28.6 | 2.4 | 12 | 57 | 27.6 | 2.4 | 13 | 57 | 26.6 | 2.4 | 14 | 57 | 25.6 | 2.4 | 15 |
| 56 | 29.6 | 2.4 | 12 | 56 | 28.6 | 2.4 | 13 | 56 | 27.6 | 2.4 | 14 | 56 | 26.6 | 2.4 | 15 |
| 55 | 30.6 | 2.4 | 12 | 55 | 29.6 | 2.4 | 13 | 55 | 28.6 | 2.4 | 14 | 55 | 27.6 | 2.4 | 15 |
| 54 | 31.6 | 2.4 | 12 | 54 | 30.6 | 2.4 | 13 | 54 | 29.6 | 2.4 | 14 | 54 | 28.6 | 2.4 | 15 |
| 53 | 32.6 | 2.4 | 12 | 53 | 31.6 | 2.4 | 13 | 53 | 30.6 | 2.4 | 14 | 53 | 29.6 | 2.4 | 15 |
| 52 | 33.6 | 2.4 | 12 | 52 | 32.6 | 2.4 | 13 | 52 | 31.6 | 2.4 | 14 | 52 | 30.6 | 2.4 | 15 |
| 51 | 34.6 | 2.4 | 12 | 51 | 33.6 | 2.4 | 13 | 51 | 32.6 | 2.4 | 14 | 51 | 31.6 | 2.4 | 15 |
| 50 | 35.6 | 2.4 | 12 | 50 | 34.6 | 2.4 | 13 | 50 | 33.6 | 2.4 | 14 | 50 | 32.6 | 2.4 | 15 |
| 49 | 36.6 | 2.4 | 12 | 49 | 35.6 | 2.4 | 13 | 49 | 34.6 | 2.4 | 14 | 49 | 33.6 | 2.4 | 15 |
| 48 | 37.6 | 2.4 | 12 | 48 | 36.6 | 2.4 | 13 | 48 | 35.6 | 2.4 | 14 | 48 | 34.6 | 2.4 | 15 |
| 47 | 38.6 | 2.4 | 12 | 47 | 37.6 | 2.4 | 13 | 47 | 36.6 | 2.4 | 14 | 47 | 35.6 | 2.4 | 15 |
| 46 | 39.6 | 2.4 | 12 | 46 | 38.6 | 2.4 | 13 | 46 | 37.6 | 2.4 | 14 | 46 | 36.6 | 2.4 | 15 |
| 45 | 40.6 | 2.4 | 12 | 45 | 39.6 | 2.4 | 13 | 45 | 38.6 | 2.4 | 14 | 45 | 37.6 | 2.4 | 15 |
| 44 | 41.6 | 2.4 | 12 | 44 | 40.6 | 2.4 | 13 | 44 | 39.6 | 2.4 | 14 | 44 | 38.6 | 2.4 | 15 |
| 43 | 42.6 | 2.4 | 12 | 43 | 41.6 | 2.4 | 13 | 43 | 40.6 | 2.4 | 14 | 43 | 39.6 | 2.4 | 15 |
| 42 | 43.6 | 2.4 | 12 | 42 | 42.6 | 2.4 | 13 | 42 | 41.6 | 2.4 | 14 | 42 | 40.6 | 2.4 | 15 |
| 41 | 44.6 | 2.4 | 12 | 41 | 43.6 | 2.4 | 13 | 41 | 42.6 | 2.4 | 14 | 41 | 41.6 | 2.4 | 15 |
| 40 | 45.6 | 2.4 | 12 | 40 | 44.6 | 2.4 | 13 | 40 | 43.6 | 2.4 | 14 | 40 | 42.6 | 2.4 | 15 |
| 39 | 46.6 | 2.4 | 12 | 39 | 45.6 | 2.4 | 13 | 39 | 44.6 | 2.4 | 14 | 39 | 43.6 | 2.4 | 15 |
| 38 | 47.6 | 2.4 | 12 | 38 | 46.6 | 2.4 | 13 | 38 | 45.6 | 2.4 | 14 | 38 | 44.6 | 2.4 | 15 |
| 37 | 48.6 | 2.4 | 12 | 37 | 47.6 | 2.4 | 13 | 37 | 46.6 | 2.4 | 14 | 37 | 45.6 | 2.4 | 15 |
| 36 | 49.6 | 2.4 | 12 | 36 | 48.6 | 2.4 | 13 | 36 | 47.6 | 2.4 | 14 | 36 | 46.6 | 2.4 | 15 |
| 35 | 50.6 | 2.4 | 12 | 35 | 49.6 | 2.4 | 13 | 35 | 48.6 | 2.4 | 14 | 35 | 47.6 | 2.4 | 15 |
| 34 | 51.6 | 2.4 | 12 | 34 | 50.6 | 2.4 | 13 | 34 | 49.6 | 2.4 | 14 | 34 | 48.6 | 2.4 | 15 |
| 33 | 52.6 | 2.4 | 12 | 33 | 51.6 | 2.4 | 13 | 33 | 50.6 | 2.4 | 14 | 33 | 49.6 | 2.4 | 15 |
| 32 | 53.6 | 2.4 | 12 | 32 | 52.6 | 2.4 | 13 | 32 | 51.6 | 2.4 | 14 | 32 | 50.6 | 2.4 | 15 |
| 31 | 54.6 | 2.4 | 12 | 31 | 53.6 | 2.4 | 13 | 31 | 52.6 | 2.4 | 14 | 31 | 51.6 | 2.4 | 15 |
| 30 | 55.6 | 2.4 | 12 | 30 | 54.6 | 2.4 | 13 | 30 | 53.6 | 2.4 | 14 | 30 | 52.6 | 2.4 | 15 |
| 60 | 25.4 | 2.6 | 12 | 60 | 24.4 | 2.6 | 13 | 60 | 23.4 | 2.6 | 14 | 60 | 22.4 | 2.6 | 15 |
| 59 | 26.4 | 2.6 | 12 | 59 | 25.4 | 2.6 | 13 | 59 | 24.4 | 2.6 | 14 | 59 | 23.4 | 2.6 | 15 |
| 58 | 27.4 | 2.6 | 12 | 58 | 26.4 | 2.6 | 13 | 58 | 25.4 | 2.6 | 14 | 58 | 24.4 | 2.6 | 15 |
| 57 | 28.4 | 2.6 | 12 | 57 | 27.4 | 2.6 | 13 | 57 | 26.4 | 2.6 | 14 | 57 | 25.4 | 2.6 | 15 |
| 56 | 29.4 | 2.6 | 12 | 56 | 28.4 | 2.6 | 13 | 56 | 27.4 | 2.6 | 14 | 56 | 26.4 | 2.6 | 15 |
| 55 | 30.4 | 2.6 | 12 | 55 | 29.4 | 2.6 | 13 | 55 | 28.4 | 2.6 | 14 | 55 | 27.4 | 2.6 | 15 |
| 54 | 31.4 | 2.6 | 12 | 54 | 30.4 | 2.6 | 13 | 54 | 29.4 | 2.6 | 14 | 54 | 28.4 | 2.6 | 15 |
| 53 | 32.4 | 2.6 | 12 | 53 | 31.4 | 2.6 | 13 | 53 | 30.4 | 2.6 | 14 | 53 | 29.4 | 2.6 | 15 |
| 52 | 33.4 | 2.6 | 12 | 52 | 32.4 | 2.6 | 13 | 52 | 31.4 | 2.6 | 14 | 52 | 30.4 | 2.6 | 15 |
| 51 | 34.4 | 2.6 | 12 | 51 | 33.4 | 2.6 | 13 | 51 | 32.4 | 2.6 | 14 | 51 | 31.4 | 2.6 | 15 |
| 50 | 35.4 | 2.6 | 12 | 50 | 34.4 | 2.6 | 13 | 50 | 33.4 | 2.6 | 14 | 50 | 32.4 | 2.6 | 15 |
| 49 | 36.4 | 2.6 | 12 | 49 | 35.4 | 2.6 | 13 | 49 | 34.4 | 2.6 | 14 | 49 | 33.4 | 2.6 | 15 |
| 48 | 37.4 | 2.6 | 12 | 48 | 36.4 | 2.6 | 13 | 48 | 35.4 | 2.6 | 14 | 48 | 34.4 | 2.6 | 15 |
| 47 | 38.4 | 2.6 | 12 | 47 | 37.4 | 2.6 | 13 | 47 | 36.4 | 2.6 | 14 | 47 | 35.4 | 2.6 | 15 |
| 46 | 39.4 | 2.6 | 12 | 46 | 38.4 | 2.6 | 13 | 46 | 37.4 | 2.6 | 14 | 46 | 36.4 | 2.6 | 15 |
| 45 | 40.4 | 2.6 | 12 | 45 | 39.4 | 2.6 | 13 | 45 | 38.4 | 2.6 | 14 | 45 | 37.4 | 2.6 | 15 |
| 44 | 41.4 | 2.6 | 12 | 44 | 40.4 | 2.6 | 13 | 44 | 39.4 | 2.6 | 14 | 44 | 38.4 | 2.6 | 15 |
| 43 | 42.4 | 2.6 | 12 | 43 | 41.4 | 2.6 | 13 | 43 | 40.4 | 2.6 | 14 | 43 | 39.4 | 2.6 | 15 |
| 42 | 43.4 | 2.6 | 12 | 42 | 42.4 | 2.6 | 13 | 42 | 41.4 | 2.6 | 14 | 42 | 40.4 | 2.6 | 15 |
| 41 | 44.4 | 2.6 | 12 | 41 | 43.4 | 2.6 | 13 | 41 | 42.4 | 2.6 | 14 | 41 | 41.4 | 2.6 | 15 |
| 40 | 45.4 | 2.6 | 12 | 40 | 44.4 | 2.6 | 13 | 40 | 43.4 | 2.6 | 14 | 40 | 42.4 | 2.6 | 15 |
| 39 | 46.4 | 2.6 | 12 | 39 | 45.4 | 2.6 | 13 | 39 | 44.4 | 2.6 | 14 | 39 | 43.4 | 2.6 | 15 |
| 38 | 47.4 | 2.6 | 12 | 38 | 46.4 | 2.6 | 13 | 38 | 45.4 | 2.6 | 14 | 38 | 44.4 | 2.6 | 15 |
| 37 | 48.4 | 2.6 | 12 | 37 | 47.4 | 2.6 | 13 | 37 | 46.4 | 2.6 | 14 | 37 | 45.4 | 2.6 | 15 |
| 36 | 49.4 | 2.6 | 12 | 36 | 48.4 | 2.6 | 13 | 36 | 47.4 | 2.6 | 14 | 36 | 46.4 | 2.6 | 15 |
| 35 | 50.4 | 2.6 | 12 | 35 | 49.4 | 2.6 | 13 | 35 | 48.4 | 2.6 | 14 | 35 | 47.4 | 2.6 | 15 |
| 34 | 51.4 | 2.6 | 12 | 34 | 50.4 | 2.6 | 13 | 34 | 49.4 | 2.6 | 14 | 34 | 48.4 | 2.6 | 15 |
| 33 | 52.4 | 2.6 | 12 | 33 | 51.4 | 2.6 | 13 | 33 | 50.4 | 2.6 | 14 | 33 | 49.4 | 2.6 | 15 |
| 32 | 53.4 | 2.6 | 12 | 32 | 52.4 | 2.6 | 13 | 32 | 51.4 | 2.6 | 14 | 32 | 50.4 | 2.6 | 15 |
| 31 | 54.4 | 2.6 | 12 | 31 | 53.4 | 2.6 | 13 | 31 | 52.4 | 2.6 | 14 | 31 | 51.4 | 2.6 | 15 |
| 30 | 55.4 | 2.6 | 12 | 30 | 54.4 | 2.6 | 13 | 30 | 53.4 | 2.6 | 14 | 30 | 52.4 | 2.6 | 15 |
| 60 | 25.2 | 2.8 | 12 | 60 | 24.2 | 2.8 | 13 | 60 | 23.2 | 2.8 | 14 | 60 | 22.2 | 2.8 | 15 |
| 59 | 26.2 | 2.8 | 12 | 59 | 25.2 | 2.8 | 13 | 59 | 24.2 | 2.8 | 14 | 59 | 23.2 | 2.8 | 15 |
| 58 | 27.2 | 2.8 | 12 | 58 | 26.2 | 2.8 | 13 | 58 | 25.2 | 2.8 | 14 | 58 | 24.2 | 2.8 | 15 |
| 57 | 28.2 | 2.8 | 12 | 57 | 27.2 | 2.8 | 13 | 57 | 26.2 | 2.8 | 14 | 57 | 25.2 | 2.8 | 15 |
| 56 | 29.2 | 2.8 | 12 | 56 | 28.2 | 2.8 | 13 | 56 | 27.2 | 2.8 | 14 | 56 | 26.2 | 2.8 | 15 |
| 55 | 30.2 | 2.8 | 12 | 55 | 29.2 | 2.8 | 13 | 55 | 28.2 | 2.8 | 14 | 55 | 27.2 | 2.8 | 15 |
| 54 | 31.2 | 2.8 | 12 | 54 | 30.2 | 2.8 | 13 | 54 | 29.2 | 2.8 | 14 | 54 | 28.2 | 2.8 | 15 |
| 53 | 32.2 | 2.8 | 12 | 53 | 31.2 | 2.8 | 13 | 53 | 30.2 | 2.8 | 14 | 53 | 29.2 | 2.8 | 15 |
| 52 | 33.2 | 2.8 | 12 | 52 | 32.2 | 2.8 | 13 | 52 | 31.2 | 2.8 | 14 | 52 | 30.2 | 2.8 | 15 |
| 51 | 34.2 | 2.8 | 12 | 51 | 33.2 | 2.8 | 13 | 51 | 32.2 | 2.8 | 14 | 51 | 31.2 | 2.8 | 15 |
| 50 | 35.2 | 2.8 | 12 | 50 | 34.2 | 2.8 | 13 | 50 | 33.2 | 2.8 | 14 | 50 | 32.2 | 2.8 | 15 |
| 49 | 36.2 | 2.8 | 12 | 49 | 35.2 | 2.8 | 13 | 49 | 34.2 | 2.8 | 14 | 49 | 33.2 | 2.8 | 15 |
| 48 | 37.2 | 2.8 | 12 | 48 | 36.2 | 2.8 | 13 | 48 | 35.2 | 2.8 | 14 | 48 | 34.2 | 2.8 | 15 |
| 47 | 38.2 | 2.8 | 12 | 47 | 37.2 | 2.8 | 13 | 47 | 36.2 | 2.8 | 14 | 47 | 35.2 | 2.8 | 15 |
| 46 | 39.2 | 2.8 | 12 | 46 | 38.2 | 2.8 | 13 | 46 | 37.2 | 2.8 | 14 | 46 | 36.2 | 2.8 | 15 |
| 45 | 40.2 | 2.8 | 12 | 45 | 39.2 | 2.8 | 13 | 45 | 38.2 | 2.8 | 14 | 45 | 37.2 | 2.8 | 15 |
| 44 | 41.2 | 2.8 | 12 | 44 | 40.2 | 2.8 | 13 | 44 | 39.2 | 2.8 | 14 | 44 | 38.2 | 2.8 | 15 |
| 43 | 42.2 | 2.8 | 12 | 43 | 41.2 | 2.8 | 13 | 43 | 40.2 | 2.8 | 14 | 43 | 39.2 | 2.8 | 15 |
| 42 | 43.2 | 2.8 | 12 | 42 | 42.2 | 2.8 | 13 | 42 | 41.2 | 2.8 | 14 | 42 | 40.2 | 2.8 | 15 |
| 41 | 44.2 | 2.8 | 12 | 41 | 43.2 | 2.8 | 13 | 41 | 42.2 | 2.8 | 14 | 41 | 41.2 | 2.8 | 15 |
| 40 | 45.2 | 2.8 | 12 | 40 | 44.2 | 2.8 | 13 | 40 | 43.2 | 2.8 | 14 | 40 | 42.2 | 2.8 | 15 |
| 39 | 46.2 | 2.8 | 12 | 39 | 45.2 | 2.8 | 13 | 39 | 44.2 | 2.8 | 14 | 39 | 43.2 | 2.8 | 15 |
| 38 | 47.2 | 2.8 | 12 | 38 | 46.2 | 2.8 | 13 | 38 | 45.2 | 2.8 | 14 | 38 | 44.2 | 2.8 | 15 |
| 37 | 48.2 | 2.8 | 12 | 37 | 47.2 | 2.8 | 13 | 37 | 46.2 | 2.8 | 14 | 37 | 45.2 | 2.8 | 15 |
| 36 | 49.2 | 2.8 | 12 | 36 | 48.2 | 2.8 | 13 | 36 | 47.2 | 2.8 | 14 | 36 | 46.2 | 2.8 | 15 |
| 35 | 50.2 | 2.8 | 12 | 35 | 49.2 | 2.8 | 13 | 35 | 48.2 | 2.8 | 14 | 35 | 47.2 | 2.8 | 15 |
| 34 | 51.2 | 2.8 | 12 | 34 | 50.2 | 2.8 | 13 | 34 | 49.2 | 2.8 | 14 | 34 | 48.2 | 2.8 | 15 |
| 33 | 52.2 | 2.8 | 12 | 33 | 51.2 | 2.8 | 13 | 33 | 50.2 | 2.8 | 14 | 33 | 49.2 | 2.8 | 15 |
| 32 | 53.2 | 2.8 | 12 | 32 | 52.2 | 2.8 | 13 | 32 | 51.2 | 2.8 | 14 | 32 | 50.2 | 2.8 | 15 |
| 31 | 54.2 | 2.8 | 12 | 31 | 53.2 | 2.8 | 13 | 31 | 52.2 | 2.8 | 14 | 31 | 51.2 | 2.8 | 15 |
| 30 | 55.2 | 2.8 | 12 | 30 | 54.2 | 2.8 | 13 | 30 | 53.2 | 2.8 | 14 | 30 | 52.2 | 2.8 | 15 |
| 60 | 25 | 3 | 12 | 60 | 24 | 3 | 13 | 60 | 23 | 3 | 14 | 60 | 22 | 3 | 15 |
| 59 | 26 | 3 | 12 | 59 | 25 | 3 | 13 | 59 | 24 | 3 | 14 | 59 | 23 | 3 | 15 |
| 58 | 27 | 3 | 12 | 58 | 26 | 3 | 13 | 58 | 25 | 3 | 14 | 58 | 24 | 3 | 15 |
| 57 | 28 | 3 | 12 | 57 | 27 | 3 | 13 | 57 | 26 | 3 | 14 | 57 | 25 | 3 | 15 |
| 56 | 29 | 3 | 12 | 56 | 28 | 3 | 13 | 56 | 27 | 3 | 14 | 56 | 26 | 3 | 15 |
| 55 | 30 | 3 | 12 | 55 | 29 | 3 | 13 | 55 | 28 | 3 | 14 | 55 | 27 | 3 | 15 |
| 54 | 31 | 3 | 12 | 54 | 30 | 3 | 13 | 54 | 29 | 3 | 14 | 54 | 28 | 3 | 15 |
| 53 | 32 | 3 | 12 | 53 | 31 | 3 | 13 | 53 | 30 | 3 | 14 | 53 | 29 | 3 | 15 |
| 52 | 33 | 3 | 12 | 52 | 32 | 3 | 13 | 52 | 31 | 3 | 14 | 52 | 30 | 3 | 15 |
| 51 | 34 | 3 | 12 | 51 | 33 | 3 | 13 | 51 | 32 | 3 | 14 | 51 | 31 | 3 | 15 |
| 50 | 35 | 3 | 12 | 50 | 34 | 3 | 13 | 50 | 33 | 3 | 14 | 50 | 32 | 3 | 15 |
| 49 | 36 | 3 | 12 | 49 | 35 | 3 | 13 | 49 | 34 | 3 | 14 | 49 | 33 | 3 | 15 |
| 48 | 37 | 3 | 12 | 48 | 36 | 3 | 13 | 48 | 35 | 3 | 14 | 48 | 34 | 3 | 15 |
| 47 | 38 | 3 | 12 | 47 | 37 | 3 | 13 | 47 | 36 | 3 | 14 | 47 | 35 | 3 | 15 |
| 46 | 39 | 3 | 12 | 46 | 38 | 3 | 13 | 46 | 37 | 3 | 14 | 46 | 36 | 3 | 15 |
| 45 | 40 | 3 | 12 | 45 | 39 | 3 | 13 | 45 | 38 | 3 | 14 | 45 | 37 | 3 | 15 |
| 44 | 41 | 3 | 12 | 44 | 40 | 3 | 13 | 44 | 39 | 3 | 14 | 44 | 38 | 3 | 15 |
| 43 | 42 | 3 | 12 | 43 | 41 | 3 | 13 | 43 | 40 | 3 | 14 | 43 | 39 | 3 | 15 |
| 42 | 43 | 3 | 12 | 42 | 42 | 3 | 13 | 42 | 41 | 3 | 14 | 42 | 40 | 3 | 15 |
| 41 | 44 | 3 | 12 | 41 | 43 | 3 | 13 | 41 | 42 | 3 | 14 | 41 | 41 | 3 | 15 |
| 40 | 45 | 3 | 12 | 40 | 44 | 3 | 13 | 40 | 43 | 3 | 14 | 40 | 42 | 3 | 15 |
| 39 | 46 | 3 | 12 | 39 | 45 | 3 | 13 | 39 | 44 | 3 | 14 | 39 | 43 | 3 | 15 |
| 38 | 47 | 3 | 12 | 38 | 46 | 3 | 13 | 38 | 45 | 3 | 14 | 38 | 44 | 3 | 15 |
| 37 | 48 | 3 | 12 | 37 | 47 | 3 | 13 | 37 | 46 | 3 | 14 | 37 | 45 | 3 | 15 |
| 36 | 49 | 3 | 12 | 36 | 48 | 3 | 13 | 36 | 47 | 3 | 14 | 36 | 46 | 3 | 15 |
| 35 | 50 | 3 | 12 | 35 | 49 | 3 | 13 | 35 | 48 | 3 | 14 | 35 | 47 | 3 | 15 |
| 34 | 51 | 3 | 12 | 34 | 50 | 3 | 13 | 34 | 49 | 3 | 14 | 34 | 48 | 3 | 15 |
| 33 | 52 | 3 | 12 | 33 | 51 | 3 | 13 | 33 | 50 | 3 | 14 | 33 | 49 | 3 | 15 |
| 32 | 53 | 3 | 12 | 32 | 52 | 3 | 13 | 32 | 51 | 3 | 14 | 32 | 50 | 3 | 15 |
| 31 | 54 | 3 | 12 | 31 | 53 | 3 | 13 | 31 | 52 | 3 | 14 | 31 | 51 | 3 | 15 |
| 30 | 55 | 3 | 12 | 30 | 54 | 3 | 13 | 30 | 53 | 3 | 14 | 30 | 52 | 3 | 15 |

### Formulations

In some embodiments, the formulations comprising the LNP and the SAM RNA or AAM RNA have a ratio of the number of nitrogen in the lipids of the LNP to the phosphorous atoms in the SAM RNA or AAM RNA from: 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, or 12.0:1. In some embodiments, the formulations comprising the LNP and the SAM RNA or AAM RNA have a ratio of the number of nitrogen in the lipids of the LNP to the phosphorous atoms in the SAM RNA or AAM RNA (N:P) to: 16.0:1, 15.9:1, 15.8:1, 15.7:1, 15.6:1, 15.5:1, 15.4:1, 15.3:1, 15.2:1, 15.1:1, 15.0:1, 14.9:1, 14.8:1, 14.7:1, 14.6:1, 14.5:1, 14.4:1, 14.3:1, 14.2:1, 14.1:1, 14.0:1, 13.9:1, 13.8:1, 13.7:1, 13.6:1, 13.5:1, 13.4:1, 13.3:1, 13.2:1, 13.1:1, 12.0:1, 12.9:1, 12.8:1, 12.7:1, 12.6:1, 12.5:1, 12.4:1, 12.3:1, 12.2:1, 12.1:1, 12.0:1, 11.9:1, 11.8:1, 11.7:1, 11.6:1, 11.5:1, 11.4:1, 11.3:1, 11.2:1, 11.1:1, 11.0:1, 10.9:1, 10.8:1, 10.7:1, 10.6:1, 10.5:1, 10.4:1, 10.3:1, 10.2:1, 10.1:1, 10.0:1, 9.9:1, 9.8:1, 9.7:1, 9.6:1, 9.5:1, 9.4:1, 9.3:1, 9.2:1, 9.1:1, 9.0:1, 8.9:1, 8.8:1, 8.7:1, 8.6:1, 8.5:1, 8.4:1, 8.3:1, 8.2:1, 8.1:1, 8.0:1, 7.9:1, 7.8:1, 7.7:1, 7.6:1, 7.5:1, 7.4:1, 7.3:1, 7.2:1, 7.1:1, 7.0:1, 6.9:1, 6.8:1, 6.7:1, 6.6:1, 6.5:1, 6.4:1, 6.3:1, 6.2:1, 6.1:1, 6.0:1, 5.9:1, 5.8:1, 5.7:1, 5.6:1, 5.5:1, 5.4:1, 5.3:1, 5.2:1, 5.1:1, 5.0:1, 4.9:1, 4.8:1, 4.7:1, 4.6:1, 4.5:1, 4.4:1, 4.3:1, 4.2:1, 4.1:1, or 4.0:1. Taking the above-noted embodiments into account, it is contemplated and supported that any of the above-noted “ratio of the number of nitrogen in the lipids of the LNP to the phosphorous atoms in the SAM RNA or AAM RNA (N:P) from” and “ratio of the number of nitrogen in the lipids of the LNP to the phosphorous atoms in the SAM RNA or AAM RNA (N:P) to” may be combined to provide an enclosed range (i.e., ratio of the number of nitrogen in the lipids of the LNP to the phosphorous atoms in the SAM RNA or AAM RNA (N:P) from 6:1 to 7:1). In some embodiments, the formulations comprising the LNP and the SAM RNA or AAM RNA have a ratio of the number of nitrogen in the lipids of the LNP to the phosphorous atoms in the SAM RNA or AAM RNA from: 2.0:1 to: 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.1:1 to: 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.2:1 to: 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.3:1 to: 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.4:1 to: 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.5:1 to: 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.6:1 to: 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.7:1 to: 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.8:1 to: 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 2.9:1 to: 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.0:1 to: 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.1:1 to: 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.2:1 to: 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.3:1 to: 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.4:1 to: 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.5:1 to: 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.6:1 to: 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.7:1 to: 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.8:1 to: 3.9:1, 4.0:1, 4.1:1, 4.2:1, 4.3:1, 4.4:1, 4.5:1, 4.6:1, 2.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 3.9:1 to: 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.0:1 to: 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.1:1 to: 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.2:1 to: 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.3:1 to: 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.4:1 to: 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.5:1 to: 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.6:1 to: 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.7:1 to: 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.8:1 to: 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 4.9:1 to: 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.0:1 to: 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.1:1 to: 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.2:1 to: 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.3:1 to: 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.4:1 to: 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.5:1 to: 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.6:1 to: 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.7:1 to: 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.8:1 to: 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 5.9:1 to: 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.0:1 to: 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.1:1 to: 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.2:1 to: 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.3:1 to: 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.4:1 to: 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.5:1 to: 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.6:1 to: 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.7:1 to: 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.8:1 to: 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 6.9:1 to: 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.0:1 to: 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.1:1 to: 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.2:1 to: 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.3:1 to: 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.4:1 to: 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.5:1 to: 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.6:1 to 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.7:1 to: 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; from 7.8:1 to: 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1; or from 7.9:1 to: 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 10.1:1, 10.2:1, 10.3:1, 10.4:1, 10.5:1, 10.6:1, 10.7:1, 10.8:1, 10.9:1, 11.0:1, 11.1:1, 11.2:1, 11.3:1, 11.4:1, 11.5:1, 11.6:1, 11.7:1, 11.8:1, 11.9:1, 12.0:1, 12.1:1, 12.2:1, 12.3:1, 12.4:1, 12.5:1, 12.6:1, 12.7:1, 12.8:1, 12.9:1, 13.0:1, 13.1:1, 13.2:1, 13.3:1, 13.4:1, 13.5:1, 13.6:1, 13.7:1, 13.8:1, 13.9:1, 14.0:1, 14.1:1, 14.2:1, 14.3:1, 14.4:1, 14.5:1, 14.6:1, 14.7:1, 14.8:1, 14.9:1, 15.0:1, 15.1:1, 15.2:1, 15.3:1, 15.4:1, 15.5:1, 15.6:1, 15.7:1, 15.8:1, 15.9:1, or 16.0:1. Also contemplated and supported are combinations of the above-noted “ratio of the number of nitrogen in the lipids of the LNP to the phosphorous atoms in the SAM RNA or AAM RNA (N:P) from” and “ratio of the number of nitrogen in the lipids of the LNP to the phosphorous atoms in the SAM RNA or AAM RNA (N:P) to” with that provided in the Examples section of this document. Also contemplated and supported are combinations of the ““ratio of the number of nitrogen in the lipids of the LNP to the phosphorous atoms in the SAM RNA or AAM RNA (N:P)” of the Example section of this document to provide a range (i.e., “ratio of the number of nitrogen in the lipids of the LNP to the phosphorous atoms in the SAM RNA or AAM RNA (N:P) from [the N:P ratio of formulation X₁]” to [N:P ratio of formulation X₂] wherein X₁ and X₂ represent any two exemplary LNP formulations of the Examples.

### Making the LNP Comprising the RNA Molecules

As noted above, In some embodiments, the SAM RNA or AAM RNA are comprised or encapsulated within the LNPs. In some embodiments, the SAM RNA or AAM RNA and lipids of the LNPs can be admixed and/or purified to thereby provide said comprising or encapsulating within. In some embodiments, the RNA molecules and lipids of the LNP can be admixed and/or purified to thereby provide the above-noted proportions of SAM RNA or AAM RNA comprised or encapsulated within the LNPs.

In some aspects, a method of obtaining a composition is provided, the composition comprising the SAM RNA or AAM RNA and LNPs, wherein the recombinant molecules are comprised or encapsulated within the LNPs in the above-noted proportions, wherein the LNPs comprise the above-noted lipids; the method comprising admixing a first solution, which comprises the SAM RNA or AAM RNA, and a second solution, which comprises the above-noted lipids. In some embodiments, the admixing is performed by at least a T-mixer, microfluidics, or an impinging jet mixer. In some embodiments, the first solution further comprises citrate buffer (e.g., sodium citrate) or acetate buffer (e.g., sodium acetate).

In some embodiments, the second solution further comprises an organic solvent. In some embodiments, the organic solvent comprises chloroform, dichloromethane, diethylether, cyclohexane, cyclopentane, benzene, toluene, methanol, benzyl alcohol, and aliphatic alcohols (e.g., C, to C₈ alcohols). In some embodiments, the aliphatic alcohols comprise ethanol, propanol, isopropanol, butanol, tert-buranol, isobutanol, pentanol, benzyl alcohol, and hexanol. In some embodiments, the organic solvent comprises an alcohol solution. In some embodiments, the organic alcohol solution comprises from 70 volume % to 100 volume % ethanol.

In some embodiments, the method comprises admixing a first solution, which comprises the SAM RNA or AAM RNA and the above-noted lipids of the LNP, and a second solution, which is an aqueous solution. In some embodiments, the RNA and lipids of the LNP are admixed in an organic solvent. In some embodiments, the organic solvent comprises chloroform, dichloromethane, diethylether, cyclohexane, cyclopentane, benzene, toluene, methanol, benzyl alcohol, and aliphatic alcohols (e.g., C, to C₈ alcohols). In some embodiments, the aliphatic alcohols comprise ethanol, propanol, isopropanol, butanol, tert-butanol, isobutanol, pentanol, benzyl alcohol, and hexanol. In some embodiments, the organic solvent comprises an alcohol solution. In some embodiments, the organic alcohol solution comprises from 70 volume % to 100 volume % ethanol. In some embodiments, the organic alcohol solution comprises from 70 volume % to 100 volume % ethanol and 30 volume % to 0 volume % benzyl alcohol. In some embodiments, the aqueous solution comprises a citrate buffer (e.g., sodium citrate) or an acetate buffer (e.g., sodium acetate). In some embodiments, the first and second solution are admixed at a ratio from 1:1 to 5:1, from 2:1 to 4:1, from 2.5:1 to 3.5:1, or at 3:1.

In some embodiments, the admixing of the first and second solutions (i.e., of either of the two above-noted methods) is at a pH from 4.5 to the pKa of the first lipid (e.g., the cation-ionizable lipid), thereby obtaining a first admixture. In some embodiments, the admixing of the first and second solutions is at a pH from 4.5, 4.6, 4.7, 4.8, 4.9, 5.0, 5.1, 5.2, 5.3, 5.4, 5.5, 5.6, 5.7, 5.8, 5.9, or 6.0 to the pKa of the first lipid (e.g., the cation-ionizable lipid), thereby obtaining a first admixture. In some embodiments, the method further comprises a first increasing, which is increasing the pH of the first admixture to be equal to or above the pKa of the first lipid to thereby obtain a pH-adjusted first admixture. In some embodiments, the first increasing obtains a pH-adjusted first admixture with a pH from the pKa of the first lipid (e.g., cation-ionizable lipid) to: 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, or 7.0.

In some embodiments, the first increasing or purifying comprises cross-flow filtration or tangential-flow filtration. In some embodiments, the first increasing or purifying further comprises transferring the composition comprising the LNPs and the SAM RNA or AAM RNA into a third solution, which differs from the first solution. In some embodiments, the third solution comprises phosphate-buffered saline. In some embodiments, the transferring comprises dialysis. In some embodiments, the tangential-flow filtration comprises the use of a hollow fiber filter. In some embodiments, the hollow fiber comprises a polyethersulfone hollow fiber filter or a polysulfone hollow fiber filter. In some embodiments, the hollow fiber filter (e.g., polyethersulfone hollow fiber filter) has a pore size cutoff of at least 75 kDa, 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, or 120 kDa. In some embodiments, the hollow fiber filter (e.g., polyethersulfone hollow fiber filter) has a pore size cutoff of no more than 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa. Taking the above-noted embodiments into account, it is contemplated and supported that any of the above-noted pore size cut offs “of at least” and “of no more than” may be combined to provide an enclosed range (i.e., a pore size cut off from 85 kDa to 120 kDa). In some embodiments, the hollow fiber filter (e.g., polyethersulfone hollow fiber filter) has a pore size cutoff of: from 75 kDa to: 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 76 kDa to: 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 77 kDa: to: 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 78 kDa to: 79 kDa, 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 79 kDa to: 80 kDa, 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 80 kDa to: 81 kDa, 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 81 kDa to: 82 kDa, 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 82 kDa to: 83 kDa, 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 83 kDa to: 84 kDa, 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 84 kDa to: 85 kDa, 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 85 kDa to: 86 kDa, 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 86 kDa to: 87 kDa, 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 87 kDa to: 88 kDa, 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 88 kDa to: 89 kDa, 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 89 kDa to: 90 kDa, 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 90 kDa to: 91 kDa, 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 91 kDa to: 92 kDa, 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 92 kDa to: 93 kDa, 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 93 kDa to: 94 kDa, 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 94 kDa to: 95 kDa, 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 95 kDa to: 96 kDa, 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 96 kDa to: 97 kDa, 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 97 kDa to: 98 kDa, 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 98 kDa to: 99 kDa, 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 99 kDa to: 100 kDa, 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 100 kDa to: 101 kDa, 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 101 kDa to: 102 kDa, 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 102 kDa to: 103 kDa, 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 103 kDa to: 104 kDa, 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 104 kDa to: 105 kDa, 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 105 kDa to: 106 kDa, 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 106 kDa to: 107 kDa, 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 107 kDa to: 108 kDa, 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 108 kDa to: 109 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 109 kDa to: 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 110 kDa to: 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 111 kDa to: 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 112 kDa to: 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 113 kDa to: 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; from 114 kDa to: 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa; or from 115 kDa to: 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, or 140 kDa.

In some embodiments, the first increasing or purifying comprises, prior to the above-noted filtrations, passing the LNP/RNA mixture through an ion exchange solid-state support. In some embodiments, the ion exchange solid-state support comprises an anion exchange column or a cation exchange column.

In some embodiments, prior to the admixing of the SAM RNA or AAM RNA and the lipids of the LNPs, the lipids of the LNP admixed with an organic solvent to obtain a concentrated stock (e.g., a stock lipid/organic solvent mixture). In some embodiments, the admixing is (e.g., the stock lipid/organic solvent mixture is stirred, rocked, vortexed, sonicated, or agitated at from 25° C. to 37° C.) for at least 5 min, 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, or 40 min to form a homogeneous stock lipid/organic solvent mixture. In some embodiments, the admixing is (e.g., the stock lipid/organic solvent mixture is stirred, rocked, vortexed, sonicated, or agitated at from 25° C. to 37° C.) for no more than 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs to form a homogeneous stock lipid/organic solvent mixture. Taking the above-noted embodiments into account, it is contemplated and supported that any of the above-noted amounts of time “of at least” and amounts of time “of no more than” may be combined to provide an enclosed range (i.e., the stock lipid/organic solvent mixture is stirred, rocked, vortexed, sonicated, or agitated at from 25° C. to 37° C. for from 5 min to 19 min). In some embodiments, the admixing is (e.g., the stock lipid/organic solvent mixture is stirred, rocked, vortexed, sonicated, or agitated at from 25° C. to 37° C.): from 5 min to: 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 6 min to: 7 min, 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 7 min to: 8 min, 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 8 min to: 9 min, 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 9 min to: 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 10 min to: 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 11 min to: 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 12 min to: 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 13 min to: 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 14 min to: 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 15 min to: 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 16 min to: 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 17 min to: 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs; from 18 min to: 19 min, 20 min, 25 min, 30 min, 35 min, 40 min, 50 min, 1 hr, 1.1 hrs, 1.2 hrs, 1.3 hrs, 1.4 hrs, or 1.5 hrs; from 19 min to: 20 min, 25 min, 30 min, 35 min, 40 min, 50 min, 1 hr, 1.1 hrs, 1.2 hrs, 1.3 hrs, 1.4 hrs, or 1.5 hrs; from 20 min to: 25 min, 30 min, 35 min, 40 min, 50 min, 1 hr, 1.1 hrs, 1.2 hrs, 1.3 hrs, 1.4 hrs, or 1.5 hrs; from 25 min to: 30 min, 35 min, 40 min, 50 min, 1 hr, 1.1 hrs, 1.2 hrs, 1.3 hrs, 1.4 hrs, or 1.5 hrs; from 30 min to: 35 min, 40 min, 50 min, 1 hr, 1.1 hrs, 1.2 hrs, 1.3 hrs, 1.4 hrs, or 1.5 hrs; from 35 min to: 35 min, 40 min, 50 min, 1 hr, 1.1 hrs, 1.2 hrs, 1.3 hrs, 1.4 hrs, or 1.5 hrs; from 40 min to: 50 min, 1 hr, 1.1 hrs, 1.2 hrs, 1.3 hrs, 1.4 hrs, or 1.5 hrs; from 50 min to: 1 hr, 1.1 hrs, 1.2 hrs, 1.3 hrs, 1.4 hrs, or 1.5 hrs; from 1 hr to: 1.1 hrs, 1.2 hrs, 1.3 hrs, 1.4 hrs, or 1.5 hrs; from 1.1 hrs to: 1.2 hrs, 1.3 hrs, 1.4 hrs, or 1.5 hrs; or from 1.2 hrs to: 1.3 hrs, 1.4 hrs, or 1.5 hrs to form a homogeneous stock lipid/organic solvent mixture. In some embodiments, the homogenous stock lipid/organic solvent mixture is further diluted in the organic solvent (i.e., ethanol) to obtain the second solution.

### Other Pharmaceutically Acceptable Delivery Vehicles

In some embodiments, the compositions do not comprise structural components of a virion, such as capsid proteins. Accordingly, the compositions do not bind to and contain the SAM RNA in a structure where they would be considered are virion particles. In some embodiments, the composition and the particle comprising the SAM RNA do not comprise a protein capsid. By avoiding the need to create a capsid particle, the methods of producing the composition do not require a packaging cell line, thus permitting easier up-scaling for commercial production and minimizing the risk that dangerous infectious viruses will inadvertently be produced.

Instead of encapsulating RNA in a virion, particles of the invention are formed from a pharmaceutically acceptable delivery vehicle. Where delivery is by polymeric microparticle, RNA can be encapsulated or adsorbed. A third delivery material of interest is the particulate reaction product of a polymer, a crosslinker, the SAM RNA, and a charged monomer.

SAM RNA can be encapsulated within the particles (particularly if the particle is a liposome or a lipid nanoparticle). This means that SAM RNA inside the particles is separated from any external medium by the pharmaceutically acceptable delivery vehicle, and encapsulation has been found to protect SAM RNA from RNase digestion. Encapsulation can take various forms. For example, in some embodiments (as in a unilamellar liposome) the delivery material forms a outer layer around an aqueous SAM RNA-containing core, whereas in other embodiments (e.g., in molded particles) the delivery material forms a matrix within which RNA is embedded. The particles can include some external SAM RNA (e.g., on the surface of the particles), but at least half of the SAM RNA (and ideally all of it) is encapsulated.

SAM RNA can be adsorbed to the particles (particularly if the particle is a polymeric microparticle). This means that RNA is not separated from any external medium by the pharmaceutically acceptable delivery vehicle, unlike for example, the RNA genome of a natural virus. The particles can include some encapsulated RNA (e.g., in the core of a particle), but at least half of the RNA (and ideally all of it) is adsorbed.

Microparticles can be made using 500 mg of PLG RG503 (50:50 lactide/glycolide molar ratio, MW of about 30 kDa) and 20 mg DOTAP using an Omni Macro Homogenizer. For example, the particle suspension can be shaken at 150 rpm overnight and then filtered through a 40 μm sterile filter for storage at 2-8° C. Self-replicating RNA was adsorbed to the particles. To prepare 1 mL of PLG/RNA suspension the required volume of PLG particle suspension can be added to a vial and nuclease-free water can be added to bring the volume to 900 μL, 100 μL SAM RNA (10 μg/mL) can be added dropwise to the PLG suspension, with constant shaking. PLG/RNA can be incubated at room temperature for 30 min. For 1 mL of reconstituted suspension, 45 mg mannitol, 15 mg sucrose and 250-500 μg of PVA can be added. The vials can be frozen at −80° C. and lyophilized.

To evaluate RNA adsorption, 100 μL particle suspension can be centrifuged at 10,000 rpm for 5 min and supernatant can be collected. PLG/RNA can be reconstituted using 1 mL nuclease-free water. To 100 μL particle suspension (1 μg RNA), 1 mg heparin sulfate can be added. The mixture can be vortexed and allowed to sit at room temperature for 30 min for RNA desorption. Particle suspension can be centrifuged, and supernatant can be collected.

For RNase stability, 100 μL particle suspension can be incubated with 6.4 mAU of RNase A at room temperature for 30 min. RNAse can be inactivated with 0.126 mAU of Proteinase K at 55° C. for 10 min. One milligram of heparin sulfate can be added to desorb the RNA followed by centrifugation. The supernatant samples containing RNA can be mixed with formaldehyde load dye, heated at 65° C. for 10 min and analyzed using a 1% denaturing gel (460 ng RNA loaded per lane).

Various polymers can form microparticles to encapsulate or adsorb RNA according to the invention. The use of a substantially non-toxic polymer means that a recipient can safely receive the particles, and the use of a biodegradable polymer means that the particles can be metabolized after delivery to avoid long-term persistence. Useful polymers are also sterilizable, to assist in preparing pharmaceutical grade formulations.

Suitable non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyester-amides, or combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone. Useful PLG polymers include those having a lactide/glycolide molar ratio ranging, for example, from 20:80 to 80:20, e.g., 25:75, 40:60, 45:55, 50:50, 55:45, 60:40, 75:25. Useful PLG polymers include those having a molecular weight between, for example, 5,000-200,000 Da e.g., between 10,000-100,000, 20,000-70,000, 30,000-40,000, 40,000-50,000 Da.

In some embodiments, the microparticles ideally have a diameter in the range of 0.02 μm to 8 μm. For a composition comprising a population of microparticles with different diameters at least 80% by number should have diameters in the range of 0.03-7 μm. In some embodiments, to facilitate adsorption of RNA, a microparticle may include a cationic surfactant and/or lipid. In some embodiments, the microparticles can have a zeta potential of between 40-100 mV.

In some embodiments, the pharmaceutically acceptable delivery vehicle comprises the particulate reaction product of a polymer, a crosslinker, a SAM RNA, and a charged monomer. These four components can be mixed as a liquid, placed in a mold (e.g., comprising a perfluoropolyether), and then cured to form the particles according to the mold's shape and dimensions. These methods provide a biodegradable crosslinked oligomeric polymer nanoparticle. In some embodiments, the particles have a largest cross-sectional dimension of 1-5 μm. They may have an overall positive charge.

In some embodiments, suitable polymers include, but are not limited to: a poly(acrylic acid); a poly(styrene sulfonate); a carboxymethylcellulose (CMC); a poly(vinyl alcohol); a poly(ethylene oxide); a poly(vinyl pyrrolidone); a dextran; or a poly(vinylpyrolidone-co-vinyl acetate-co-vinyl alcohol). In some embodiments, the polymer is a poly(vinyl pyrrolidinone). The amount of polymer for forming the particles can be between 2-75 wt %, e.g., 10-60 wt %, 20-60 wt %.

In some embodiments, the compositions comprise, and thereby the particles comprise, suitable crosslinkers such as a disulfide and/or ketal. In some embodiments, the crosslinker can comprise poly(epsilon-caprolactone)-b-tetraethylene glycol-b-poly(epsilon-capro lactone)dimethacrylate, poly(epsilon-caprolactone)-b-poly(ethylene glycol)-b-poly(epsilon-capro lactone)dimethacrylate, poly(lactic acid)-b-tetraethylene glycol-b-poly(lactic acid)dimethacrylate, poly(lactic acid)-b-poly(ethylene glycol)-b-poly(lactic acid)dimethacrylate, poly(glycolic acid)-b-tetraethylene glycol-b-poly(glycolic acid)dimethacrylate, poly(gly colic acid)-b-poly(ethylene glycol)-b-poly(gly colic acid)dimethacrylate, poly(epsilon-caprolactone)-b-tetraethylene glycol-b-poly(epsilon-caprolactone)diacrylate, poly(epsilon-caprolactone)-b-poly(ethylene glycol)-b-poly(epsilon-caprolactone)diacrylate, poly(lactic acid)-b-tetraethylene glycol-b-poly(lactic acid)diacrylate, poly(lactic acid)-b-poly(ethylene glycol)-b-poly(lactic acid)diacrylate, poly(glycolic acid)-b-tetraethylene glycol-b-poly(glycolic acid)diacrylate, poly(glycolic acid)-b-poly(ethylene glycol)-b-poly(glycolic acid)diacrylate, silane, silicon containing methacrylates, or dimethyldi(methacryloyloxy-1-ethoxy)silane. The amount of crosslinker for forming the particles can be between 10-25 wt %, e.g., 10-60 wt %, 20-60 wt %.

In some embodiments, the charged monomers can be cationic or anionic. These include but are not limited to: [2-(acryloyloxy)ethyl]trimethyl ammonium chloride (AETMAC) and 2-aminoethyl methacrylate hydrochloride (AEM-HCl). The amount of charged monomer for forming the particles can be between 2-75 wt %.

In some embodiments, the amount of RNA for forming the particles can be between 0.25-20 wt %. In some embodiments, a pre-cure mixture inside a mold can include an initiator. For instance, the mold can include 0.1-1 wt % initiator, 0.1-0.5 wt % initiator, or 0.1 wt % initiator. Between 0.1-0.5% initiator is useful. Photoinitiators such as DEAP and DPT are useful e.g., for use with ultraviolet curing.

### Methods of Administering the SAM RNA

In one aspect, a method of eliciting an immune response in a subject to an immunogen is provided, wherein the method comprises administering to the subject an effective amount of the SAM RNA or the composition, wherein the heterologous nucleic acid encodes a heterologous protein, wherein the heterologous protein comprises the immunogen or an antibody against the immunogen. In some embodiments, the immune response is a protective immune response. In some embodiments, the immune response being a therapeutic immune response. In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen.

In another aspect, a method of delivering an inhibitory RNA in the SAM RNA to a subject is provided, wherein the method comprises administering to the subject an effective amount of the SAM RNA or a composition comprising the SAM RNA, and wherein the first RNA segment comprises the inhibitory RNA. In some embodiments, the inhibitory RNA is against a native messenger RNA encoding a native protein, and the administering decreasing the expression of the native protein in the subject when compared to the expression of the native protein in the subject without the administering. In another aspect, a method of delivering to a subject a heterologous nucleic acid in the SAM RNA or the composition comprising the SAM RNA is provided, wherein the method comprises administering an effective amount of the SAM RNA. In some embodiments, the subject is human. In some embodiments, the subject is a mammal, such as a human or a large veterinary mammal (e.g., horses, cattle, deer, goats, pigs). Where the composition or SAM RNA is for eliciting a protective immune response, the subject is preferably a human, such as a child (e.g., a toddler or infant), a teenager, and the SAM RNA or the compositions comprising the SAM RNA is formulated as a vaccine. Where the composition or SAM RNA is used as a treatment or for therapeutic use, the human is preferably a teenager or an adult. A vaccine intended for children may also be administered to adults.

In some embodiments, the SAM RNA or composition comprising the SAM RNA is administered to the subject intramuscularly, intradermally, subcutaneously, transcutaneously, topically, intraperitoneally, intrathecally, pulmonarily (i.e., inhaled), intracerebroventricularly, intravenously, intra-arterially, onto a mucosa (i.e., vaginally), buccally, sublingually, intranasally, optically, to the cornea, or into the eyeball.

In some embodiments, a method for treating cancer in a subject is provided, the subject having a tumor of the cancer, the method comprising administering to the subject the SAM RNA, or an effective amount thereof, the first RNA encoding a heterologous protein comprising a polyepitopic peptide comprising two or more immunogenic neo-epitopes and a linker, the linker linking the two or more immunogenic neo-epitopes, the immunogenic neo-epitopes being from a first sample comprising cells from the tumor from the subject, each neo-epitope being: (a) encoded in mRNA in the first sample, (b) occurring in a protein-coding region therein, (c) being predicted to bind to a major histocompatibility complex, and (d) introduces a difference in the amino acid sequence of the neo-epitope when compared to a reference amino acid sequence or genetic sequence predicted to encode the reference amino acid sequence obtained from a second sample from a non-cancerous cell from the subject. In some embodiments, the SAM RNA is produced from a method comprising: obtaining a first nucleic acid sequence from the first sample, obtaining a second nucleic acid sequence from the second sample, comparing the first nucleic acid sequence to the second nucleic acid sequence thereby obtaining at least two somatic mutations present in the tumor cells, identifying from the at least two somatic mutations (a)-(d), and producing the SAM RNA.

In one aspect, a method for treating cancer in a subject is provided, the method comprising administering to the subject the SAM RNA, or an effective amount thereof, the heterologous protein comprising IL-12sc, IL-15sushi, IFNα, or GM-CSF. In some embodiments, the method further comprises administering an anti-PD-1/PD-L1 checkpoint inhibitor. In some embodiments the cancer is melanoma, Head and Neck Squamous Cell Cancer (HNSCC), Cutaneous Squamous Cell Carcinoma (CSCC), or advanced anti-PD-1/PD-L1 naïve cancers thereof.

In some embodiments, a method for treating cancer in a subject is provided, the method comprising administering to the subject the SAM RNA, or an effective amount thereof, the heterologous protein comprising autogene cevumeran or atezolizumag. In some embodiments the cancer is melanoma, head and neck squamous cell cancer (HNSCC), cutaneous squamous cell carcinoma (CSCC), non-small cell lung cancer (NSCLC), or advanced anti-PD-1/PD-L1 naïve cancers thereof.

In some embodiments, the effective amount comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of SAM RNA or AAM RNA per administration. Taking the above-noted embodiments into account, it is contemplated and supported that any of the above-noted “μg” of “at least” and any of the above-noted “μg” of “no more than” may be combined to provide an enclosed range (i.e., the effective amount comprises or is from 25 μg to 75 μg of SAM RNA or AAM RNA per administration). In some embodiments, the effective amount comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per administration.

Also contemplated and supported are combinations of effective amounts of “at least” and any of the above-noted effective amounts of “no more than” with the effective amounts provided in an Example of this document. Also contemplated and supported are combinations of the effective amounts of the Example section of this document to provide a range (i.e., the effective amount from [the effective amount from example X₁] to [the effective amount from example X₂] wherein X₁ and X₂ represent any two exemplary treatments of the Examples.

In some embodiments, the above-noted methods comprise, is, or consist of more than one administration (i.e., two, three, four, five, six, or seven administrations). In some embodiments, the above-noted methods comprise, is, or consist of at least 2, 3, 4, 5, 6, 7, 8, 9, or 10 administrations (i.e., two, three, four, five, six, or seven administrations). In some embodiments, the above-noted methods comprise, is, or consist of no more than 15, 14, 13, 12, 11, 10, 9, 8, 7, 6, 5, 4, 3, or 2 administrations (i.e., two, three, four, five, six, or seven administrations). Taking the above-noted embodiments into account, it is contemplated and supported that any of the above-noted number of administrations of “at least” and “no more than” may be combined to provide an enclosed range (i.e., from 8 to 15 administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 administrations.

Also contemplated and supported are combinations of number of administrations of “at least” and number of administrations of “no more than” with the number of administrations provided in an Example of this document. Also contemplated and supported are combinations of the number of administrations of the Example section of this document to provide a range (i.e., the number of administrations from [the number of administrations from example X₁] to [number of administrations of example X₂] wherein X₁ and X₂ represent any two exemplary treatments of the Examples.

In some embodiments, the above-noted methods comprise, is, or consist of more than one administration of an effective amount (i.e., two, three, four, five, six, or seven administrations). In some embodiments, the above-noted methods comprise, is, or consist of at least 2, 3, 4, 5, 6, 7, 8, 9, or 10 administrations of an effective amount (i.e., two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 administrations of an effective amount (i.e., two, three, four, five, six, or seven administrations of an effective amount). Taking the above-noted embodiments into account, it is contemplated and supported that any of the above-noted number of administrations of the effective amount of “at least” and “no more than” may be combined to provide an enclosed range (i.e., from 8 to 15 administrations of the effective amount). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 administrations of the effective amount.

Also contemplated and supported are combinations of number of administrations of “at least” and number of administrations of an effective amount of “no more than” with the number of administrations of an effective amount provided in an Example of this document. Also contemplated and supported are combinations of the number of administrations of an effective amount of the Example section of this document to provide a range (i.e., the number of administrations from [the number of administrations from example X₁] to [number of administrations of example X₂] wherein X₁ and X₂ represent any two exemplary treatments of the Examples.

In some embodiments, the administration comprises a primary administration and a booster administration. In some embodiments, the effective amount differs between the primary administration and the booster administration.

In some embodiments, the above-noted methods comprise, is, or consist of more than one primary administration (i.e., two, three, four, five, six, or seven administrations). In some embodiments, the above-noted methods comprise, is, or consist of at least 2, 3, 4, 5, 6, 7, 8, 9, or 10 primary administrations (i.e., two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 primary administrations (i.e., two, three, four, five, six, or seven administrations of an effective amount). Taking the above-noted embodiments into account, it is contemplated and supported that any of the above-noted number of primary administrations of “at least” and “no more than” may be combined to provide an enclosed range (i.e., from 8 to 15 primary administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 primary administrations.

Also contemplated and supported are combinations of the number of primary administrations of “at least” and the number of primary administrations of “no more than” with the number of primary administrations provided in an Example of this document. Also contemplated and supported are combinations of the number of primary administrations of the Example section of this document to provide a range (i.e., the number of primary administrations from [the number of primary administrations from example X₁] to [number of primary administrations of example X₂] wherein X₁ and X₂ represent any two exemplary treatments of the Examples.

In some embodiments, the primary administration comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per primary administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of SAM RNA or AAM RNA per primary administration. Taking the above-noted embodiments into account, it is contemplated and supported that any of the above-noted “μg” of “at least” and any of the above-noted “μg” of “no more than” may be combined to provide an enclosed range (i.e., the primary administration comprises or is from 25 μg to 75 μg of SAM RNA or AAM RNA per primary administration). In some embodiments, the primary administration comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per primary administration.

Also contemplated and supported are combinations of primary administrations of “at least” and any of the above-noted primary administrations of “no more than” with the primary administrations provided in an Example of this document. Also contemplated and supported are combinations of the primary administrations of the Example section of this document to provide a range (i.e., the primary administration from [the primary administration from example X₁] to [the primary administration from example X₂] wherein X₁ and X₂ represent any two exemplary treatments of the Examples.

In some embodiments, the immune response that the primary administration elicits comprises a cell-mediated immune response, an antibody-response (e.g., humoral immune response), a T_{H1} immune response, or a T_{H2} immune response. In some embodiments, the eliciting of the immune response by the primary administration is from a naïve immune response where the immune system has no detectable antibody response that is against the immunogen, cell-mediated immune response that is against the immunogen, T_{H1} immune response that is against the immunogen, or a T_{H2} immune response that is against the immunogen to a cell-mediated immune response that is against the immunogen, an antibody-response (e.g., humoral immune response) that is against the immunogen, a T_{H1} immune response that is against the immunogen, or a T_{H2} immune response that is against the immunogen. In some embodiments, the eliciting of the immune response by the primary administration is from an experienced immune response where the antibody response that is against the immunogen, the cell-mediated immune response that is against the immunogen, the T_{H1} immune response that is against the immunogen, or the T_{H2} immune response that is against the immunogen is unable to reduce the likelihood of at least one symptom of the disease or from being infectious to others by the disease to a cell-mediated immune response that is against the immunogen, an antibody-response (e.g., humoral immune response) that is against the immunogen, a T_{H1} immune response that is against the immunogen, or a T_{H2} immune response that is against the immunogen that reduces the likelihood of at least one symptom of the disease or the likelihood of being infectious to others by the disease. In some embodiments, at least one symptom of the disease comprises death, respiratory distress, reduced blood oxygen, fever, chills, a febrile response, shortness of breath, difficulty breathing, fatigue, muscle aches, body aches, headaches, new or loss of smell, mucus production, sore throat, congestion, runny nose, nausea, vomiting, diarrhea, confusion, pressure in the chest, persistent pain, inability to wake, inability to stay awake, pale skin, gray skin, blue-colored skin, pale lips, gray lips, blue-colored lips, pale nail-beds, gray nail-beds, blue-colored nail-beds, low reperfusion of extremities, low reperfusion of the body, encephalitis, stroke, ischemia, fibromyalgia, or myocarditis.

In some embodiments, the above-noted methods comprise, is, or consist of more than one booster administration (i.e., two, three, four, five, six, or seven administrations). In some embodiments, the above-noted methods comprise, is, or consist of at least 2, 3, 4, 5, 6, 7, 8, 9, or 10 booster administrations (i.e., two, three, four, five, six, or seven administrations of an effective amount). In some embodiments, the above-noted methods comprise, is, or consist of no more than 15, 14, 13, 12, 11, 10, 9, 8, 7, 6, 5, 4, 3, or 2 booster administrations (i.e., two, three, four, five, six, or seven administrations of an effective amount). Taking the above-noted embodiments into account, it is contemplated and supported that any of the above-noted number of booster administrations of “at least” and “no more than” may be combined to provide an enclosed range (i.e., from 8 to 15 booster administrations). In some embodiments, the above-noted methods comprise, is, or consists of from 1 to: 2, 3, 4, 5, 6, 7, 8, 9, 10, 11, 12, 13, 14, or 15; from 2 to: 3, 4, 5, 6, 7, 8, 9, 10, 11, 12, 13, 14, or 15; 3 to: 4, 5, 6, 7, 8, 9, 10, 11, 12, 13, 14, or 15; from 4 to: 5, 6, 7, 8, 9, 10, 11, 12, 13, 14, or 15; from 5 to: 6, 7, 8, 9, 10, 11, 12, 13, 14, or 15; or from 6 to: 7, 8, 9, 10, 11, 12, 13, 14, or 15 booster administrations.

Also contemplated and supported are combinations of the number of booster administrations of “at least” and the number of booster administrations of “no more than” with the number of booster administrations provided in an Example of this document. Also contemplated and supported are combinations of the number of booster administrations of the Example section of this document to provide a range (i.e., the number of booster administrations from [the number of booster administrations from example X₁] to [number of booster administrations of example X₂] wherein X₁ and X₂ represent any two exemplary treatments of the Examples.

In some embodiments, the booster administration comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per booster administration. In some embodiments, the effective amount comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of SAM RNA or AAM RNA per booster administration. Taking the above-noted embodiments into account, it is contemplated and supported that any of the above-noted “μg” of “at least” and any of the above-noted “μg” of “no more than” may be combined to provide an enclosed range (i.e., the booster administration comprises or is from 25 μg to 75 μg of SAM RNA or AAM RNA per booster administration). In some embodiments, the booster administration comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA per booster administration.

Also contemplated and supported are combinations of booster administrations of “at least” and any of the above-noted booster administrations of “no more than” with the booster administrations provided in an Example of this document. Also contemplated and supported are combinations of the booster administrations of the Example section of this document to provide a range (i.e., the booster administration from [the booster administration from example X₁] to [the booster administration from example X₂] wherein X₁ and X₂ represent any two exemplary treatments of the Examples.

In some embodiments, the immune response that the booster administration elicits comprises a cell-mediated immune response, an antibody-response (e.g., humoral immune response), a T_{H1} immune response, or a T_{H2} immune response. In some embodiments, the eliciting of the immune response by the booster administration is from an experienced immune response where the antibody response that is against the immunogen, the cell-mediated immune response that is against the immunogen, the T_{H1} immune response that is against the immunogen, or the T_{H2} immune response that is against the immunogen is unable to reduce the likelihood of at least one symptom of the disease or from being infectious to others by the disease to a cell-mediated immune response that is against the immunogen, an antibody-response (e.g., humoral immune response) that is against the immunogen, a T_{H1} immune response that is against the immunogen, or a T_{H2} immune response that is against the immunogen that reduces the likelihood of at least one symptom of the disease or the likelihood of being infectious to others by the disease. In some embodiments, the eliciting of the immune response by the booster administration is from an experienced immune response where the antibody response that is against the immunogen, the cell-mediated immune response that is against the immunogen, the T_{H1} immune response that is against the immunogen, or the T_{H2} immune response that is against the immunogen is, at the time of booster administration, able to reduce the likelihood of at least one symptom of the disease or from being infectious to others by the disease, but some time thereafter and without the booster administration, would not be able to reduce the likelihood of at least one symptom of the disease or from being infectious to others by the disease (i.e., a boosting of the duration of the immune response). In such cases, the booster administration elicits an immune response when compared to the same individual who would not have received the booster administration and who would have otherwise not been able to reduce the likelihood of at least one symptom of the disease or from being infectious to others by the disease (i.e., boosted duration). In some embodiments of such cases, the eliciting of the immune response by the booster administration comprises a renewed cell-mediated immune response that is against the immunogen, a renewed antibody-response (e.g., humoral immune response) that is against the immunogen, a renewed T_{H1} immune response that is against the immunogen, or a renewed T_{H2} immune response that is against the immunogen that reduces the likelihood of at least one symptom of the disease or the likelihood of being infectious to others by the disease during the period in which, without the booster administration, the subject would have otherwise had an increased likelihood of at least one symptom of the disease or an increased likelihood of being infectious to the disease (i.e., boosted duration). In some embodiments, the booster administration elicits an immune response that is above the immune response elicited by the primary administration. In some embodiments, the immune response that is elicited by the booster administration is an antibody response that is above that elicited by the primary administration, a cell-mediated immune response that is above that elicited by the primary administration, a T_{H1} immune response that is above that elicited by the primary administration, or a T_{H2} immune response that is above that elicited by the primary administration. In some embodiments, at least one symptom of the disease comprises death, respiratory distress, reduced blood oxygen, fever, chills, a febrile response, shortness of breath, difficulty breathing, fatigue, muscle aches, body aches, headaches, new or loss of smell, mucus production, sore throat, congestion, runny nose, nausea, vomiting, diarrhea, confusion, pressure in the chest, persistent pain, inability to wake, inability to stay awake, pale skin, gray skin, blue-colored skin, pale lips, gray lips, blue-colored lips, pale nail-beds, gray nail-beds, blue-colored nail-beds, low reperfusion of extremities, low reperfusion of the body, encephalitis, stroke, ischemia, fibromyalgia, or myocarditis.

In one aspect, a method of eliciting an immune response in a subject to an immunogen is provided, wherein the method comprises administering to the subject a unit dose of the SAM RNA or AAM RNA or the formulation. A “unit dose” is contemplated and understood to be that dose provided in an administration, and several unit doses (i.e., the unit doses from several administrations may be combined) to make a total dose administered to the subject. In some embodiments, the immune response is a protective immune response. In some embodiments, the immune response or the protective immune response comprises a cell-mediated immune response, an antibody-response (e.g., humoral immune response), a T_{H1} immune response, or a T_{H2} immune response. In some embodiments, the immune response is a therapeutic immune response. In some embodiments, the heterologous polypeptide comprises the antibody against the immunogen. In some embodiments, the heterologous polypeptide comprises the immunogen.

In another aspect, a method of delivering to a subject a heterologous nucleic acid in the recombinant RNA or a formulation comprising the recombinant RNA is provided, wherein the method comprises administering an unit dose of the recombinant RNA.

In some embodiments, the subject is human. In some embodiments, the subject is a mammal, such as a human or a large veterinary mammal (e.g., horses, cattle, deer, goats, pigs). Where the formulation or recombinant RNA is for eliciting a protective immune response, the subject is preferably a human, such as a child (e.g., a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an unit dose consistent with the state of the immune system of the subject (i.e., the elderly having more difficulty eliciting certain immune responses) and the average body weight of a subject of that age or the actual body weight of the subject.

In some embodiments, the recombinant RNA or formulation comprising the recombinant RNA is administered to the subject intramuscularly, intradermally, subcutaneously, transcutaneously, topically, intraperitoneally, intrathecally, pulmonarily (i.e., inhaled), intracerebroventricularly, intravenously, intra-arterially, onto a mucosa (i.e., vaginally), buccally, sublingually, intranasally, optically, to the cornea, or into the eyeball.

In some embodiments, a method for treating cancer in a subject is provided, the subject having a tumor of the cancer, the method comprising administering to the subject the formulation comprising the recombinant RNA or the recombinant RNA or an unit dose thereof. In some embodiments, the recombinant RNA comprises a sequence that encodes a heterologous polypeptide comprising a polyepitopic peptide comprising two or more immunogenic neo-epitopes and a linker, the linker linking the two or more immunogenic neo-epitopes, the immunogenic neo-epitopes being from a first sample comprising cells from the tumor from the subject, each neo-epitope being: (a) encoded in mRNA in the first sample, (b) occurring in a protein-coding region therein, (c) being predicted to bind to a major histocompatibility complex, and (d) being capable of introducing a difference in the amino acid sequence of the neo-epitope when compared to a reference amino acid sequence or genetic sequence predicted to encode the reference amino acid sequence obtained from a second sample from a non-cancerous cell from the subject. In some embodiments, the recombinant RNA is produced from a method comprising: obtaining a first nucleic acid sequence from the first sample, obtaining a second nucleic acid sequence from the second sample, comparing the first nucleic acid sequence to the second nucleic acid sequence thereby obtaining at least two somatic mutations present in the tumor cells, identifying from the at least two somatic mutations (a)-(d), and producing the recombinant RNA.

In one aspect, a method for treating cancer in a subject is provided, the method comprising administering to the subject the recombinant RNA, a formulation comprising the recombinant RNA, or an unit dose thereof, wherein the recombinant RNA comprises a sequence encoding a heterologous polypeptide comprising IL-12sc, IL-15sushi, IFNα, or GM-CSF. In some embodiments, the method further comprises administering an anti-PD-1/PD-L1 checkpoint inhibitor. In some embodiments, the cancer is melanoma, head and neck squamous cell cancer (HNSCC), cutaneous squamous cell carcinoma (CSCC), or advanced anti-PD-1/PD-L1 naïve cancers thereof.

In some embodiments, a method for treating cancer in a subject is provided, the method comprising administering to the subject the recombinant RNA, a formulation comprising the recombinant RNA, or an unit dose thereof. In some embodiments, the recombinant RNA comprises a sequence encoding a heterologous polypeptide comprising autogene, cevumeran, or atezolizumag. In some embodiments, the cancer is melanoma, head and neck squamous cell cancer (HNSCC), cutaneous squamous cell carcinoma (CSCC), non-small cell lung cancer (NSCLC), or advanced anti-PD-1/PD-L1 naïve cancers thereof.

In some embodiments, the unit dose comprises or is at least: 0.1 μg, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA. In some embodiments, the unit dose comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg, 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82 μg, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg, 56 μg, 55 μg, 54 μg, 53 μg, 52 μg, 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg, 21 μg, 20 μg, 19 μg, 18 μg, 17 μg, 16 μg, or 15 μg of SAM RNA or AAM RNA. Taking the above-noted embodiments into account, it is contemplated and supported that any of the above-noted “μg” of “at least” and any of the above-noted “μg” of “no more than” may be combined to provide an enclosed range (i.e., the unit dose comprises or is from 25 μg to 75 μg). In some embodiments, the unit dose comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 2 μg to: 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 4 μg to: 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 6 μg to: 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 8 μg to: 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 10 μg to: 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 11 μg to: 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 12 μg to: 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 13 μg to: 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 14 μg to: 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 16 μg to: 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 18 μg to: 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 20 μg to: 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 21 μg to: 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 22 μg to: 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 24 μg to: 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 25 μg to: 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 26 μg to: 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 28 μg to: 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 30 μg to: 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 32 μg to: 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 34 μg to: 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 35 μg to: 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 36 μg to: 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 38 μg to: 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 40 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 42 μg to: 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 44 μg to: 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 46 μg to: 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 48 μg to: 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 49 μg to: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 50 μg to: 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 52 μg to: 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 53 μg to: 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 54 μg to: 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 56 μg to: 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 58 μg to: 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 60 μg to: 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 62 μg to: 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 64 μg to: 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 66 μg to: 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 68 μg to: 69 μg 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 69 μg to: 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 70 μg to: 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 72 μg to: 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 74 μg to: 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 76 μg to: 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 77 μg to: 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 78 μg to: 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 80 μg to: 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of SAM RNA or AAM RNA.

Also contemplated and supported are combinations of unit doses of “at least” and any of the above-noted unit doses of “no more than” with the unit doses provided in an Example of this document. Also contemplated and supported are combinations of the unit doses of the Example section of this document to provide a range (i.e., the unit dose from [the unit dose from example X₁] to [the unit dose from example X₂] wherein X₁ and X₂ represent any two exemplary treatments of the Examples.

### Processes of Manufacture/Formulation of SAM RNA

In some aspects, a method of manufacturing the SAM RNA is provided. In one embodiment, the method comprises admixing an RNA polymerase, the N1-methylpseudouridines, the uridines, and a template nucleic acid comprising a sequence of the SAM RNA, thereby obtaining an admixture. In some embodiments, the template nucleic acid comprises SEQ ID NO.: 2 or 5. In some embodiments, the admixture has a second mole percentage of the N1-methylpseudouridines to the total of the N1-methylpseudouridines and the uridines. In some embodiments, the second mole percentage is the same as the first mole percentage. Accordingly, it is contemplated that the above-noted first mole percentage may also be used to determine the second mole percentages of the N1-methylpseudouridines to the total of the N1-methylpseudouridines and the uridines in the admixture. In some embodiments, the admixing is under conditions wherein the RNA polymerase produces the SAM RNA from the template nucleic acid. In some embodiments, the N1-methylpseudouridines in the admixtures are N1-methylpseudouridine triphosphates.

In some embodiments, the uridine in the admixture are uridine triphosphates. In some embodiments, the N1-methylpseudouridine triphosphates and/or the uridine triphosphates are converted to the N1-methylpseudouridines and/or the uridines respectively by the RNA polymerase (i.e., DNA-dependent RNA polymerase or RNA-dependent RNA polymerase) as they are incorporated into the newly synthesized strand of RNA.

As noted above, in some embodiments, the SAM RNA comprises a 5′ cap, which may be a cap-0, cap-1, or cap-2. Accordingly, in one aspect is provided a method of producing a SAM RNA comprising a 5′ cap, which may be a cap-0, cap-1, or cap-2. In some embodiments, the method comprises:
a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the first admixing being under conditions wherein the RNA polymerase produces the SAM RNA from the template nucleic acid, thereby obtaining an uncapped SAM RNA; and
a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.

In some embodiments, the RNA polymerase is a T7 RNA polymerase. As noted above, in some embodiments a composition comprising the SAM RNA and a pharmaceutically acceptable vehicle is provided. In some embodiments, the method comprises encapsulating the SAM RNA in the pharmaceutically acceptable delivery vehicle or adsorbing the SAM RNA to the pharmaceutically acceptable delivery vehicle. In some embodiments, the encapsulation can occur by spray-jet of two or more solutions, such as a first solution and a second solution. In some embodiments, the first solution comprises the SAM RNA, and in some embodiments the second solution comprises the lipids and other lipophilic components of the lipid nanoparticles suspended in water, at a pH of about 5.5. In some embodiments, the two solutions are spray jet sprayed at one another to form nanoparticles. In some embodiments the pH is then adjusted to be around 6.5. In some embodiments, the pH is further adjusted to be around 7.4 to form compositions of nanoparticles having a narrower range of diameters.

### Uses to Form Medicaments Comprising the SAM RNA

In one aspect, a use of the SAM RNA for the manufacture of a medicament for delivering the heterologous nucleic acid to a subject in need thereof is provided; the use comprising admixing the SAM RNA with a pharmaceutically acceptable delivery vehicle. In some embodiments, the pharmaceutically acceptable delivery vehicle comprises a lipid nanoparticle (LNP). In some embodiments, the LNP encapsulates the SAM RNA. In some embodiments, the subject in need thereof has cancer, requires cell-replacement therapy, is deficient in an enzyme, is undergoing cytokine release syndrome, has Neiman-Pick disease, has galactosemia, including classic galactosemia, Duarte (D/D) galactosemia, or Duarte/classical variant (D/G) galactosemia.

In some embodiments, the first RNA or first RNA segment comprises two or more heterologous nucleic acids. In some embodiments, the heterologous nucleic acid encodes one or more heterologous proteins. In some embodiments, the heterologous protein is extended pharmacokinetic (PK) interleukin (IL)-2 or extended pharmacokinetic (PK) interleukin (IL)-7. In some embodiments, the heterologous protein is a peptide or protein comprising an epitope for inducing an immune response against an antigen in the subject. In some embodiments, first RNA or first RNA segment encodes a heterologous protein, which is a peptide or protein comprising an epitope for inducing an immune response against an antigen in the subject, and a heterologous protein, which is extended pharmacokinetic (PK) interleukin (IL)-2 or extended pharmacokinetic (PK) interleukin (IL)-7. In some embodiments, the heterologous protein is a first fusion protein comprising extended PK-IL-2 or extended-PK IL-7. In some embodiments, the first fusion protein comprises an IL2 moiety and a moiety selected from: serum albumin, an immunoglobulin fragment, transferrin, Fn3, variants thereof, and combinations thereof.

In some embodiments, the heterologous protein comprises interleukin-12sc (IL-12sc), IL-15sushi, IFNα, or GM-CSF. In some embodiments, the heterologous protein comprise octamer-binding transcription factor-3/4 (OCT3/4), (sex determining region Y)-box-2 (SOX-2), Kruppel-like factor-4 (KLF-4), cellular myelocytomatosis oncogene (c-MYC), LIN-28, or NANOG. In some embodiments, the heterologous protein further comprises a differentiation factor, which differentiates a pluripotent cell into at least one of a myocyte, a neurocyte, a pancreatic cell, a hepatocyte, a spleen cell, a bone marrow cell, or a skin cell.

In some embodiments, the heterologous protein comprises an antibody. In some embodiments, the antibody is tocilizumab or etanercept. In some embodiments, the antibody is an anti-IL6, anti-IL6R, anti-TNF-α, or anti-TNF receptor.

In some embodiments, the heterologous protein comprises an immunotherapeutic molecule or an enzyme. In some embodiments, the enzyme comprises a galactose-1-phosphate uridylyltransferase (GALT) or acid sphingomyelinase, which would be administered to a subject having reduced or deficient activity for the native enzyme, i.e., someone having galactosemia or Neiman-Pick disease respectively. In some embodiments, the heterologous protein is a coagulation factor. In some embodiments, the coagulation factor is protein C thrombomodulin, Protein S, active protein C, Factor I, Factor IA, Factor Prothombin, Factor Thrombin, Antithrombin, Tissue Factor, Factor VII, Factor VIIa, Factor X, Factor Xa, Factor XI, Factor XIa, Factor XII, Factor XIIa, Factor XIII, Factor XIIIa, Factor IX, Factor IXa, Factor VIII, Factor VIIIa, Factor XII, Factor XIIa, Factor FV, or Factor FVa.

In one aspect, a use of the SAM RNA for the manufacture of a medicament for preventing a disease caused by a pathogen is provided; the pathogen comprising an immunogen; the use comprising admixing the SAM RNA with a pharmaceutically acceptable delivery vehicle. In some embodiments, the pharmaceutically acceptable delivery vehicle comprises a lipid nanoparticle (LNP). In some embodiments, the LNP encapsulates the SAM RNA. In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen.

In one aspect, a use of the SAM RNA for the manufacture of a medicament for treating a disease caused by a pathogen is provided; the pathogen comprising an immunogen; the use comprising admixing the SAM RNA with a pharmaceutically acceptable delivery vehicle. In some embodiments, the pharmaceutically acceptable delivery vehicle comprises a lipid nanoparticle (LNP). In some embodiments, the LNP encapsulates the SAM RNA. In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen.

### SAM RNA for Use in, for Example, Eliciting an Immune Response, Preventing Infection, or Treating Infection

In one aspect, a SAM RNA for use in eliciting an immune response to an antigen in a subject is provided. In some embodiments, the SAM RNA comprises N1-methylpseudorudines, uridines, an RNA segment that encodes the antigen, and an RNA segment that encodes one or more proteins capable of replicating the SAM RNA in an intracellular environment. In some embodiments, the SAM RNA has a first mole percentage of the N1-methylpseudouridines to the total of the N1-methylpseudouridines and the uridines from 15% to 75%.

In another aspect, a SAM RNA for use in preventing infection by a pathogen in a subject is provided. In some embodiments, the pathogen produces an antigen. In some embodiments, the SAM RNA comprises N1-methylpseudorudines, uridines, an RNA segment that encodes the antigen, and an RNA segment that encodes one or more proteins capable of replicating the SAM RNA in an intracellular environment. In some embodiments, the SAM RNA has a first mole percentage of the N1-methylpseudouridines to the total of the N1-methylpseudouridines and the uridines from 15% to 75%.

In another aspect, a SAM RNA for use in treating infection by a pathogen in a subject is provided. In some embodiments, the pathogen produces an antigen. In some embodiments, the SAM RNA comprises N1-methylpseudorudines, uridines, an RNA segment that encodes the antigen, and an RNA segment that encodes one or more proteins capable of replicating the SAM RNA in an intracellular environment. In some embodiments, the SAM RNA has a first mole percentage of the N1-methylpseudouridines to the total of the N1-methylpseudouridines and the uridines from 15% to 75%.

In some embodiments of the three above-noted aspects, the subject is a human. In some embodiments of the three above-noted aspects, the first mole percentage comprises any of the above noted “to” and above noted “from” mole percentages for the embodiments to SAM RNA. In some embodiments of the three above-noted aspects, the first mole percentage is to: 70%, 65%, 60%, 55%, or 50%. In some embodiments of the three above-noted aspects, the first mole percentage is from: 20% or 25%. In some embodiments of the three above-noted aspects, the one or more proteins capable of replicating the SAM RNA in an intracellular environment comprises an alphavirus non-structural protein-1 (nsP1), an alphavirus nsP2, an alphavirus nsP3, and an alphavirus nsP4. In some embodiments of the three above-noted aspects, the one or more proteins capable of replicating the SAM RNA in an intracellular environment comprises an alphavirus non-structural protein-1 (nsP1), an alphavirus nsP2, an alphavirus nsP3, or an alphavirus nsP4.

In some embodiments of the three above-noted aspects, the SAM RNA for the above-noted uses may comprise any of the above-noted components of the SAM RNA described above in any of the above-noted configurations. That is, for example, it may further comprise: a poly-adenosine monophosphate (poly(A)) tail; a 5′ untranslated region (5′ UTR), the 5′ UTR being 5′ of the first RNA segment and the second RNA segment; a 3′ untranslated region (3′ UTR), the 3′ UTR being 3′ of the first RNA segment and the second RNA segment, and optionally being 5′ of the poly(A) tail; a 5′ cap, optionally wherein the 5′ cap is a cap-0, a cap-1, or a cap-2.

In some embodiments of the three above-noted aspects, composition comprising the SAM RNA for the above-noted uses and a pharmaceutically acceptable delivery vehicle are provided. In some embodiments, the delivery vehicle comprises a lipid nanoparticle (LNP). In some embodiments, the LNP encapsulates the SAM RNA.

A method of manufacturing the SAM RNA of any of the above noted embodiments is also provided, as well as methods of manufacturing compositions comprising the SAM RNA. In some embodiments, the method comprises admixing an RNA polymerase, the N1-methylpseudouridines, the uridines, and a template nucleic acid comprising a sequence of the SAM RNA, thereby obtaining an admixture. In some embodiments, the admixture comprises a second mole percentage of the N1-methylpseudouridines to the total of the N1-methylpseudouridines and the uridines being the same as the first mole percentage, which is the mole percentage of the N1-methylpseudouridines to the total of the N1-methylpseudouridines and the uridines in the SAM RNA. Accordingly, it is contemplated that the above-noted first mole percentage may also be used to determine the second mole percentages of the N1-methylpseudouridines to the total of the N1-methylpseudouridines and the uridines in the admixture. In some embodiments, the admixing is under conditions wherein the RNA polymerase produces the SAM RNA from the template nucleic acid.

As noted above, in some embodiments, the SAM RNA comprises a 5′ cap, which may be a cap-0, cap-1, or cap-2. Accordingly, in one aspect is provided a method of producing a SAM RNA comprising a 5′ cap, which may be a cap-0, cap-1, or cap-2. In some embodiments, the method comprises:
a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage, which is the mole percentage of the N1-methylpseudouridines to the total of the N1-methylpseudouridines and the uridines in the SAM RNA; the first admixing being under conditions wherein the RNA polymerase produces the SAM RNA from the template nucleic acid, thereby obtaining an uncapped SAM RNA; and
a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.

In some embodiments, the RNA polymerase is a T7 RNA polymerase.

In another aspect, a method of manufacturing any one of the above-noted compositions is provided; the method comprising encapsulating the SAM RNA in the pharmaceutically acceptable delivery vehicle or adsorbing the SAM RNA to the pharmaceutically acceptable delivery vehicle.

### SAM RNA for Use in Delivering

In another aspect, a SAM RNA for use in delivering a heterologous nucleic acid to a subject is provided or a composition comprising said SAM RNA for said use is provided; the SAM RNA comprises N1-methylpseudorudines, uridines, a RNA segment that comprises a heterologous nucleic acid, and an RNA segment that encodes one or more proteins capable of replicating the SAM RNA in an intracellular environment. In some embodiments, the SAM RNA has a first mole percentage of the N1-methylpseudouridines to the total of the N1-methylpseudouridines and the uridines from 15% to 75%. In some embodiments, the first mole percentages for the other described SAM RNA, regardless of their use. In some embodiments, the subject is a human. In some embodiments, the first mole percentage is to: 70%, 65%, 60%, 55%, or 50%. In some embodiments, the first mole percentage is from: 20% or 25%. In some embodiments, the one or more proteins capable of replicating the SAM RNA in an intracellular environment comprises an alphavirus non-structural protein-1 (nsP1), an alphavirus nsP2, an alphavirus nsP3, and an alphavirus nsP4. In some embodiments, the one or more proteins capable of replicating the SAM RNA in an intracellular environment comprises an alphavirus non-structural protein-1 (nsP1), an alphavirus nsP2, an alphavirus nsP3, or an alphavirus nsP4. In some embodiments, the SAM RNA further comprises a poly-adenosine monophosphate (poly(A)) tail. In some embodiments, the SAM RNA further comprises a 5′ untranslated region (5′ UTR). In some embodiments, the 5′ UTR is 5′ of the first RNA segment and the second RNA segment. In some embodiments, the SAM RNA further comprises a 3′ untranslated region (3′ UTR). In some embodiments, the 3′ UTR is 3′ of the first RNA segment and the second RNA segment, and optionally is 5′ of the poly(A) tail.

In some embodiments, the heterologous nucleic acid encodes a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody, an antibody against the immunogen, an immunotherapeutic molecule, or an antibody against an immune signaling molecule. In some embodiments, the heterologous protein comprises an immunogen or an antibody against the immunogen. In some embodiments, the SAM RNA further comprises a 5′ cap. In some embodiments, the 5′ cap being a cap-0, a cap-1, or a cap-2. In one aspect, a composition comprising the SAM RNA and a pharmaceutically acceptable delivery vehicle. In some embodiments, the pharmaceutically acceptable delivery vehicle comprises a lipid nanoparticle (LNP), which optionally encapsulates the SAM RNA.

### SAM RNA Having a Mole Percentage Determined by the Admixture Used to Obtain it, Methods of Use, and Methods of Manufacture

As noted above, in some aspects, provided is a SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture. In some embodiments, the admixture has a first mole percentage of the N1-methylpseudouridines to the total of the N1-methylpseudouridines and the uridines from 15% to 75%. In some embodiments, the admixture has a first mole proportion of the N1-methylpseudouridines to the total of the N1-methylpseudouridines and the uridines from 0.15 to 0.75. In some embodiments, the admixture has a first mole ratio of the N1-methylpseudouridines to the uridines from 15:85 to 75:25. In some embodiments, the admixing being under conditions wherein the RNA polymerase produces the SAM RNA from the template nucleic acid. In some embodiments, the first mole percentage is any one of the embodiments provided in the section above. In some embodiments, the first mole proportion is any one of the embodiments provided in the section above. In some embodiments, the first mole ratio is any one of the embodiments provided in the section above. In some embodiments, the first mole percentage is to: 70%, 65%, 60%, 55%, or 50%. In some embodiments, the first mole percentage is from: 20% or 25%.

In some embodiments, the one or more proteins capable of replicating the SAM RNA in an intracellular environment comprising an alphavirus non-structural protein-1 (nsP1), an alphavirus nsP2, an alphavirus nsP3, and an alphavirus nsP4. In some embodiments, the one or more proteins capable of replicating the SAM RNA in an intracellular environment comprising an alphavirus non-structural protein-1 (nsP1), an alphavirus nsP2, an alphavirus nsP3, or an alphavirus nsP4. In some embodiments, the heterologous nucleic acid encodes a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody, an antibody against the immunogen, an immunotherapeutic molecule, or an antibody against an immune signaling molecule. In some embodiments, the heterologous protein comprises an immunogen or an antibody against the immunogen. In some embodiments, the SAM RNA further comprises a poly-adenosine monophosphate (poly(A)) tail. In some embodiments, the SAM RNA further comprises a 5′ untranslated region (5′ UTR). In some embodiments, the 5′ UTR is 5′ of the first RNA segment and the second RNA segment. In some embodiments, the SAM RNA further comprises a 3′ untranslated region (3′ UTR). In some embodiments, the 3′ UTR is 3′ of the first RNA segment and the second RNA segment, and optionally is 5′ of the poly(A) tail.

In some embodiments, the heterologous protein comprises an immunogen or an antibody against the immunogen. In some embodiments, the SAM RNA further comprises a 5′ cap. In some embodiments, the 5′ cap being a cap-0, a cap-1, or a cap-2.

In another aspect, a composition is provided; the composition comprising above-embodied SAM RNA and a pharmaceutically acceptable delivery vehicle. In some embodiments, the pharmaceutically acceptable delivery vehicle comprises a lipid nanoparticle (LNP). In some embodiments, the LNP encapsulates the SAM RNA.

In one aspect, a method of eliciting an immune response in a subject to an immunogen is provided; the method comprising administering to the subject an effective amount of the SAM RNA, wherein the heterologous nucleic acid encodes the immunogen or an antibody against the immunogen. In some embodiments, the immune response is a protective immune response. In some embodiments, the immune response is a therapeutic immune response. In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen. In one aspect, a method of delivering the inhibitory RNA in the above-noted SAM RNA to a subject, the method comprises administering to the subject an effective amount of the SAM RNA.

In one aspect, a method of delivering to a subject the inhibitory RNA in any of the above-noted compositions, the method comprises administering to the subject an effective amount of the composition. In some embodiments, the inhibitory RNA is against a endogenous messenger RNA encoding a endogenous protein of the subject, and the administering decreasing the expression of the endogenous protein in the subject when compared to the expression of the endogenous protein in the subject without the administering.

In one aspect, a method of delivering to a subject the heterologous nucleic acid in the above-noted SAM RNA is provided; the method comprises administering an effective amount of the SAM RNA. In one aspect, a method of delivering to a subject the heterologous nucleic acid in any of the above-noted compositions is provided; the method comprises administering to the subject an effective amount of the composition. In one aspect, a method of manufacturing any of the above-noted compositions is provided; the method comprises encapsulates the SAM RNA in the pharmaceutically acceptable delivery vehicle or adsorbing the SAM RNA to the pharmaceutically acceptable delivery vehicle.

In one aspect, a use of the above-noted SAM RNA for the manufacture of a medicament for delivering the heterologous nucleic acid is provided; the use comprising admixing the SAM RNA with a pharmaceutically acceptable delivery vehicle.

In one aspect, a use of the above-noted SAM RNA for the manufacture of a medicament for preventing a disease caused by a pathogen is provided; the pathogen comprising an immunogen; the use comprises admixing the SAM RNA with a pharmaceutically acceptable delivery vehicle.

In one aspect, a use of the above-noted SAM RNA for the manufacture of a medicament for treating a disease caused by a pathogen is provided; the pathogen comprising an immunogen; the use comprises admixing the SAM RNA with a pharmaceutically acceptable delivery vehicle.

In some embodiments of any of the above-noted uses, the pharmaceutically acceptable delivery vehicle comprises a lipid nanoparticle (LNP). In some embodiments of the above-noted use, the LNP encapsulates the SAM RNA. In some embodiments of any of the above-noted uses, the immunogen comprises a venom, a poison, an allergen, a cancer antigen, a bacterial antigen, a viral antigen, a fungal antigen, a parasite antigen, or a fragment thereof. In some embodiments of any of the above-noted uses, the heterologous protein comprises the antibody against the immunogen. In some embodiments of any of the above-noted uses, the heterologous protein comprises the immunogen.

In one aspect, a SAM RNA for use in eliciting an immune response to an antigen in a subject is provided; the SAM RNA comprises N1-methylpseudouridines, uridines, an RNA segment that encodes the antigen, and an RNA segment that encodes one or more proteins capable of replicating the SAM RNA in an intracellular environment. In some embodiments, the SAM RNA is produced by a method, which comprises admixing an RNA polymerase, the N1-methylpseudouridines, the uridines, and a template nucleic acid comprises a sequence of the SAM RNA, thereby obtaining an admixture. In some embodiments, the admixture has a mole percentage of N1-methylpseudouridines to the total of the N1-methylpseudouridines and the uridines from 15% to 75%; the admixing is under conditions wherein the RNA polymerase produces the SAM RNA from the template nucleic acid.

In one aspect, a SAM RNA for use in preventing infection by a pathogen in a subject is provided; the pathogen producing an antigen; the SAM RNA comprising N1-methylpseudorudines, uridines, an RNA segment that encodes the antigen, and an RNA segment that encodes one or more proteins capable of replicating the SAM RNA in an intracellular environment. In some embodiments, the SAM RNA is produced by a method, which comprises admixing an RNA polymerase, the N1-methylpseudouridines, the uridines, and a template nucleic acid comprises a sequence of the SAM RNA, thereby obtaining an admixture. In some embodiments, the admixture has a mole percentage of N1-methylpseudouridines to the total of the N1-methylpseudouridines and the uridines from 15% to 75%; the admixing is under conditions wherein the RNA polymerase produces the SAM RNA from the template nucleic acid.

In one aspect, a SAM RNA for use in treating infection by a pathogen in a subject is provided; the pathogen producing an antigen; the SAM RNA comprises N1-methylpseudorudines, uridines, an RNA segment that encodes the antigen, and an RNA segment that encodes one or more proteins capable of replicating the SAM RNA in an intracellular environment. In some embodiments, the SAM RNA is produced by a method, which comprises admixing an RNA polymerase, the N1-methylpseudouridines, the uridines, and a template nucleic acid comprises a sequence of the SAM RNA, thereby obtaining an admixture. In some embodiments, the admixture has a first mole percentage of N1-methylpseudouridines to the total of the N1-methylpseudouridines and the uridines from 15% to 75%; the admixing is under conditions wherein the RNA polymerase produces the SAM RNA from the template nucleic acid.

In some embodiments of any of the three above-noted aspects, the subject is a human. In some embodiments, the first mole percentage is to: 70%, 65%, 60%, or 55%. In some embodiments of any of the three above-noted aspects, the first mole percentage is from: 20% or 25%. In some embodiments of any of the three above-noted aspects, the one or more proteins capable of replicating the SAM RNA in an intracellular environment comprises an alphavirus non-structural protein-1 (nsP1), an alphavirus nsP2, an alphavirus nsP3, and an alphavirus nsP4. In some embodiments of any of the three above-noted aspects, the one or more proteins capable of replicating the SAM RNA in an intracellular environment comprises an alphavirus non-structural protein-1 (nsP1), an alphavirus nsP2, an alphavirus nsP3, or an alphavirus nsP4.

In some embodiments of any of the three above-noted aspects, the SAM RNA further comprises a poly-adenosine monophosphate (poly(A)) tail. In some embodiments of any of the three above-noted aspects, the SAM RNA further comprises a 5′ untranslated region (5′ UTR). In some embodiments of any of the three above-noted aspects, the 5′ UTR is 5′ of the first RNA segment and the second RNA segment. In some embodiments of any of the three above-noted aspects, the SAM RNA further comprises a 3′ untranslated region (3′ UTR). In some embodiments of any of the three above-noted aspects, the 3′ UTR is 3′ of the first RNA segment and the second RNA segment, and optionally is 5′ of the poly(A) tail.

In some embodiments of any of the three above-noted aspects, the SAM RNA further comprises a 5′ cap. In some embodiments of any of the three above-noted aspects, the 5′ cap is a cap-0, a cap-1, or a cap-2. In some embodiments of any of the three above-noted aspects, the 5′ cap is a cap-1. In some embodiments of any of the three above-noted aspects, the 5′ cap is a cap-0.

In some embodiments of any of the three above-noted aspects, the RNA polymerase is a T7 RNA polymerase.

In one aspect, a composition is provided; the composition comprising any of the embodiments of any of the three above-noted aspects SAM RNA and a pharmaceutically acceptable delivery vehicle. In some embodiments, the delivery vehicle comprises a lipid nanoparticle (LNP). In some embodiments, the LNP encapsulates the SAM RNA.

In one aspect, a method of manufacturing any of the above-noted compositions is provided; the method comprising encapsulating the SAM RNA in the pharmaceutically acceptable delivery vehicle or adsorbing the SAM RNA to the pharmaceutically acceptable delivery vehicle.

In one aspect, a SAM RNA for use in delivering a heterologous nucleic acid to a subject is provided; the SAM RNA comprising N1-methylpseudorudines, uridines, a first RNA segment, and a second RNA segment. In some embodiments, the first RNA segment comprises a heterologous nucleic acid. In some embodiments, the second RNA segment encodes one or more proteins capable of replicating the SAM RNA in an intracellular environment. In some embodiments, the SAM RNA is produced by a method, which comprises admixing an RNA polymerase, the N1-methylpseudouridines, the uridines, and a template nucleic acid comprises a sequence of the SAM RNA, thereby obtaining an admixture. In some embodiments, the admixture having a first mole percentage of N1-methylpseudouridines to the total of the N1-methylpseudouridines and the uridines from 15% to 75%; the admixing is under conditions wherein the RNA polymerase produces the SAM RNA from the template nucleic acid.

In some embodiments, the subject is a human. In some embodiments, the first mole percentage is to: 70%, 65%, 60%, 55%, or 50%. In some embodiments, the first mole percentage is from: 20% or 25%.

In some embodiments, the one or more proteins capable of replicating the SAM RNA in an intracellular environment comprises an alphavirus non-structural protein-1 (nsP1), an alphavirus nsP2, an alphavirus nsP3, and an alphavirus nsP4. In some embodiments, the one or more proteins capable of replicating the SAM RNA in an intracellular environment comprises an alphavirus non-structural protein-1 (nsP1), an alphavirus nsP2, an alphavirus nsP3, or an alphavirus nsP4. In some embodiments, the above-noted SAM RNA further comprises a poly-adenosine monophosphate (poly(A)) tail. In some embodiments, the above-noted SAM RNA comprises a 5′ untranslated region (5′ UTR). In some embodiments, the 5′ UTR is 5′ of the first RNA segment and the second RNA segment. In some embodiments, the above-noted SAM RNA further comprises a 3′ untranslated region (3′ UTR). In some embodiments, the 3′ UTR is 3′ of the first RNA segment and the second RNA segment, and optionally is 5′ of the poly(A) tail.

In some embodiments, the heterologous nucleic acid encodes a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody against the immunogen, an immunotherapeutic molecule, or an antibody against an immune signaling molecule. In some embodiments, the heterologous protein comprises an immunogen or an antibody against the immunogen.

In some embodiments, the above-noted SAM RNA further comprises a 5′ cap. In some embodiments, the 5′ cap is a cap-0, a cap-1, or a cap-2. In some embodiments the 5′ cap is a cap-1. In some embodiments, the 5′ cap is a cap-0.

In one aspect, a composition comprising the above-noted SAM RNA and a pharmaceutically acceptable delivery vehicle is provided. In some embodiments, the delivery vehicle comprises a lipid nanoparticle (LNP). In some embodiments, the LNP encapsulates the SAM RNA.

### Pluralities of Auto-Amplifying Messenger (AAM) RNAs, Methods of Use, and Methods of Manufacture

In one aspect, a plurality of auto-amplifying messenger (AAM) RNAs is provided; the AAM RNAs comprising N1-methylpseudouridines, uridines, a first RNA, and one or more second RNA. In some embodiments, the first RNA comprises a heterologous nucleic acid. In some embodiments, the one or more second RNA encodes one or more proteins capable of replicating the SAM RNA in an intracellular environment. In some embodiments, the plurality of AAM RNAs has a first mole percentage of the N1-methylpseudouridines to the total of the N1-methypseudouridines and uridines from 15% to 75%. In some embodiments. In some embodiments, the plurality of AAM RNAs has a first mole percentage of the N1-methylpseudouridines to the total of the N1-methypseudouridines and uridines from 15% to 75%.

In some embodiments, the plurality of AAM RNAs has a first mole proportion of the N1-methylpseudouridines to the total of the N1-methylpseudouridines and the uridines from 0.15 to 0.75. In some embodiments, the first mole percentage is any one of the embodiments provided in the section above. In some embodiments, the first mole proportion is any one of the embodiments provided in the section above. In some embodiments, the first mole ratio is any one of the embodiments provided in the section above. In some embodiments, the plurality of AAM RNAs has a first mole ratio of the N1-methylpseudouridines to the uridines from 15:85 to 75:25.

In some embodiments, the first RNA comprises the N1-methylpseudouridines and the uridines. In some embodiments, the one or more second RNA comprises the N1-methylpseudouridines and the uridines. In some embodiments, the first mole percentage is to 70, 65, 60, 55, or 50%. In some embodiments, the first mole percentage is from 20% or 25%.

In some embodiments, the one or more proteins that together are capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments, the heterologous nucleic acid encoding a heterologous protein. In some embodiments, the heterologous nucleic acid comprises an inhibitory RNA. In some embodiments of the above-noted plurality of AAM RNAs, the inhibitory RNA comprises an antisense RNA, a small interfering RNA, or a microRNA. In some embodiments, the heterologous protein comprises an immunogen, an antibody, or an immunotherapeutic molecule. In some embodiments, the heterologous protein comprises an immunogen or an antibody against the immunogen. In some embodiments, the plurality of AAM RNAs further comprises a poly-adenosine monophosphate (poly(A)) tail.

In some embodiments, the plurality of AAM RNAs further comprises a 5′ untranslated region (5′ UTR). In some embodiments, the 5′ UTR is 5′ of the first RNA or the second RNA. In some embodiments, the plurality of AAM RNAs further comprises a 3′ untranslated region (3′ UTR). In some embodiments, the 3′ UTR is 3′ of the first RNA or the second RNA, and optionally is 5′ of the poly(A) tail.

In some embodiments, the plurality of AAM RNAs further comprises a 5′ cap. In some embodiments, the 5′ cap is a cap-0, a cap-1, or a cap-2.

In one aspect, a composition comprising any of the above-noted pluralities of AAM RNAs and a pharmaceutically acceptable delivery vehicle is provided. In some embodiments, the pharmaceutically acceptable delivery vehicle comprises a lipid nanoparticle (LNP). In some embodiments, the LNP encapsulates at least one of the one or more first RNA or at least one of the one.

In one aspect, a method of eliciting an immune response in a subject to the immunogen is provided; the method comprising administering to the subject an effective amount or any of the above-noted compositions. In some embodiments, the immune response is a protective immune response. In some embodiments, the immune response is a therapeutic immune response. In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen.

In one aspect, a method of delivering the inhibitory RNA in any of the above-noted pluralities of AAM RNAs to a subject is provided; the method comprising administering to the subject an effective amount of the plurality of AAM RNAs.

In one aspect, a method of delivering to a subject the inhibitory RNA in any of the above-noted compositions is provided; the method comprising administering to the subject an effective amount of the composition.

In some embodiments, the inhibitory RNA is against a endogenous messenger RNA encoding a endogenous protein of the subject, and the administering decreasing the expression of the endogenous protein in the subject when compared to the expression of the endogenous protein in the subject without the administering.

In one aspect, a method of delivering to a subject the heterologous nucleic acid in any of the above-noted pluralities of AAM RNAs is provided; the method comprising administering an effective amount of the plurality of AAM RNAs.

In one aspect, a method of delivering to a subject the heterologous nucleic acid in any of the above-noted compositions is provided; the method comprising administering to the subject an effective amount of the composition.

In some embodiments, the subject is human.

In one aspect, a method of manufacturing any of the above-noted pluralities of AAM RNAs is provided; the method comprising one or more admixing of an RNA polymerase, the N1-methylpseudouridines, the uridines, and one or more template nucleic acids, thereby obtaining an admixture wherein the one or more template nucleic acids comprise a sequence of the plurality of AAM RNAs. In some embodiments, the one or more admixture has a second mole percentage of the N1-methylpseudouridines to the total of the N1-methylpseudouridines and the uridines. In some embodiments, the second mole percentage is the same as the first mole percentage. In some embodiments, the one or more admixing is under conditions wherein the RNA polymerase produces the plurality of AAM RNAs from the one or more template nucleic acids.

In some embodiments, the RNA polymerase is a T7 RNA polymerase.

In one aspect, a method of manufacturing any of the above-noted pluralities of AAM RNAs comprising a 5′ cap is provided; the method comprising:
one or more first admixing of an RNA polymerase, the N1-methylpseudouridines, the uridines, and one or more template nucleic acids, thereby obtaining one or more first admixtures wherein the one or more template nucleic acids comprise a sequence of the plurality of AAM RNAs; the one or more first admixture having a second mole percentage of the N1-methylpseudouridines to the total of the N1-methylpseudouridines and the uridines; the second mole percentage is the same as the first mole percentage, which is the mole percentage of the N1-methylpseudouridines to the total of the N1-methylpseudouridines and the uridines in the SAM RNA; the one or more first admixing is under conditions wherein the RNA polymerase produces a plurality of uncapped AAM RNAs from the one or more template nucleic acids, wherein the plurality of uncapped AAM RNA have the sequence of the plurality of the AAM RNA; and
a one or more second admixing of the plurality of the uncapped AAM RNAs, a messenger RNA guanylyltransferase, guanosine triphosphate, a (guaninine-N7-)-methyltransferase, and S-adenosyl-L-methionine, the one or more second admixing is under conditions that cause a 5′ to 5′ triphosphate cap linkage, the one or more second admixing optionally further comprises a 2′-O-methyltransferase, and optionally the one or more second admixing is under conditions that form cap-1 or cap-2.

In some embodiments, the RNA polymerase is a T7 RNA polymerase.

In one aspect, a method of manufacturing any of the above-noted compositions is provided; the method comprising encapsulating the plurality of AAM RNAs in the pharmaceutically acceptable delivery vehicle or adsorbing the plurality of AAM RNAs to the pharmaceutically acceptable delivery vehicle.

A use of any of the above-noted pluralities of AAM RNAs for the manufacture of a medicament for delivering the heterologous nucleic acid is provided; the use comprising admixing the plurality of AAM RNAs with a pharmaceutically acceptable delivery vehicle.

A use of any of the above-noted pluralities of AAM RNAs for the manufacture of a medicament for preventing a disease caused by a pathogen is provided; the pathogen comprising the immunogen; the use comprises admixing the plurality of AAM RNAs with a pharmaceutically acceptable delivery vehicle.

A use of any of the above-noted pluralities of AAM RNAs for the manufacture of a medicament for treating a disease caused by a pathogen is provided; the pathogen comprises the immunogen; the use comprising admixing the plurality of AAM RNAs with a pharmaceutically acceptable delivery vehicle.

In some embodiments of any of the above-noted uses, the pharmaceutically acceptable delivery vehicle comprises a lipid nanoparticle (LNP). In some embodiments of the above-noted use, the LNP encapsulates the plurality of AAM RNAs. In some embodiments of any of the above-noted uses, the immunogen comprises a venom, a poison, an allergen, a cancer antigen, a bacterial antigen, a viral antigen, a fungal antigen, a parasite antigen, or a fragment thereof. In some embodiments of any of the above-noted uses, the heterologous protein comprises the antibody against the immunogen. In some embodiments of any of the above-noted uses, the heterologous protein comprises the immunogen.

In one aspect, a plurality of AAM RNAs for use in eliciting an immune response to an antigen in a subject is provided; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

In one aspect, a plurality of AAM RNAs for use in preventing infection by a pathogen in a subject is provided; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

In one aspect, a plurality of AAM RNAs for use in treating infection by a pathogen in a subject is provided; the pathogen producing an antigen; the plurality of AAM RNAs comprising N1-methylpseudorudines, uridines, a first RNA, and one or more second RNA; the first RNA encoding the antigen; the one or more second RNA encoding one or more proteins capable of replicating the plurality of AAM RNAs in an intracellular environment; the plurality of AAM RNAs having a first mole percentage of the N1-methylpseudouridines to the total of the N1-methylpseudouridines and the uridines from 15% to 75%.

In some embodiments of any of the above-noted pluralities of AAM RNAs for the above noted uses, the subject is a human. In some embodiments of any of the above-noted pluralities of AAM RNAs, the first RNA comprises the N1-methylpseudouridines and the uridines. In some embodiments of any of the above-noted pluralities of AAM RNAs, the one or more second RNA comprises the N1-methylpseudouridines and the uridines. In some embodiments of any of the above-noted pluralities of AAM RNAs, the first mole percentage is to: 70%, 65%, 60%, 55, or 50%. In some embodiments of any of the above-noted pluralities of AAM RNAs, the first mole percentage is from 20% or 25%.

In some embodiments of any of the above-noted pluralities of AAM RNAs, the one or more proteins capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments of any of the above-noted pluralities of AAM RNAs, the one or more proteins capable of replicating the plurality of AAM RNAs in an intracellular environment comprises an alphavirus non-structural protein-1 (nsP1), an alphavirus nsP2, an alphavirus nsP3, or an alphavirus nsP4.

In some embodiments of any of the above-noted pluralities of AAM RNAs further comprises a poly-adenosine monophosphate (poly(A)) tail. In some embodiments of any of the above-noted pluralities of AAM RNAs further comprises a 5′ untranslated region (5′ UTR). In some embodiments, the 5′ UTR is 5′ of the first RNA or the second RNA. In some embodiments of any of the above-noted pluralities of AAM RNAs further comprises a 3′ untranslated region (3′ UTR). In some embodiments, the 3′ UTR is 3′ of the first RNA or the second RNA, and optionally is 5′ of the poly(A) tail.

In some embodiments of any of the above-noted pluralities of AAM RNAs further comprises a 5′ cap. In some embodiments of the above-noted plurality of AAM RNAs, the 5′ cap is a cap-0, a cap-1, or a cap-2.

In one aspect, a composition is provided; the composition comprising any of the above-noted pluralities of AAM RNAs and a pharmaceutically acceptable delivery vehicle. In some embodiments, the delivery vehicle comprises a lipid nanoparticle (LNP). In some embodiments, the LNP encapsulates the plurality of AAM RNAs.

In one aspect, a method of manufacturing any of the above-noted pluralities of AAM RNAs is provided; the method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a second mole percentage of the N1-methylpseudouridines to the total of the N1-methylpseudouridines and the uridines. In some embodiments, the second mole percentage is the same as the first mole percentage. In some embodiments, the one or more admixing is under conditions wherein the RNA polymerase produces the plurality of AAM RNAs from the one or more template nucleic acids.

In some embodiments, the RNA polymerase is a T7 RNA polymerase.

In one aspect, a method of manufacturing any of the above-noted pluralities of AAM RNAs comprising a 5′ cap is provided; the method comprising:
one or more first admixing of an RNA polymerase, the N1-methylpseudouridines, the uridines, and one or more template nucleic acids, thereby obtaining one or more first admixtures wherein the one or more template nucleic acids comprise a sequence of the plurality of AAM RNAs; the one or more first admixtures having a second mole percentage of the N1-methylpseudouridines to the total of the N1-methylpseudouridines and the uridines; the second mole percentage is the same as the first mole percentage, which is the mole percentage of the N1-methylpseudouridines to the total of the N1-methylpseudouridines and the uridines in the SAM RNA; the one or more first admixing is under conditions wherein the RNA polymerase produces a plurality of uncapped AAM RNAs from the one or more template nucleic acids, wherein the plurality of uncapped AAM RNA have the sequence of the plurality of the AAM RNA; and
one or more second admixing of the plurality of uncapped AAM RNAs, a messenger RNA guanylyltransferase, guanosine triphosphate, a (guaninine-N7-)-methyltransferase, and S-adenosyl-L-methionine, the one or more second admixing is under conditions that cause a 5′ to 5′ triphosphate cap linkage, the one or more second admixing optionally further comprises a 2′-O-methyltransferase, and optionally the one or more second admixing is under conditions that form cap-1 or cap-2.

In some embodiments, the RNA polymerase is a T7 RNA polymerase.

In one aspect, a method of manufacturing any of the above-noted compositions is provided; the method comprising encapsulating the plurality of AAM RNAs in the pharmaceutically acceptable delivery vehicle or adsorbing the plurality of AAM RNAs to the pharmaceutically acceptable delivery vehicle.

In one aspect, a plurality of AAM RNAs for use in delivering a heterologous nucleic acid to a subject is provided; the plurality of AAM RNAs comprising N1-methylpseudorudines, uridines, a first RNA, and one or more second RNA. In some embodiments, the first RNA comprises a heterologous nucleic acid. In some embodiments, the one or more second RNA encoding one or more proteins capable of replicating the plurality of AAM RNAs in an intracellular environment. In some embodiments, the plurality of AAM RNAs having a first mole percentage of the N1-methylpseudouridines to the total of the N1-methylpseudouridines and the uridines from 15% to 75%.

In some embodiments, the subject is a human. In some embodiments of any of the above-noted plurality of AAM RNAs, the first RNA comprises the N1-methylpseudouridines and the uridines. In some embodiments, the one or more second RNA comprises the N1-methylpseudouridines and the uridines. In some embodiments, the first mole percentage is to 70%. In some embodiments, the first mole percentage of the plurality is contemplated and supported to be any of the above-disclosed ranges of first mole percentages as described for the SAM RNA. In some embodiments, the first mole percentage is to 65%. In some embodiments, the first mole percentage is to 60%. In some embodiments, the first mole percentage is to 55%. In some embodiments, the first mole percentage is to 50%. In some embodiments, the first mole percentage is from 20%. In some embodiments, the first mole percentage is from 25%.

In some embodiments, the one or more proteins capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments, the one or more proteins capable of replicating the plurality of AAM RNAs in an intracellular environment comprises an alphavirus non-structural protein-1 (nsP1), an alphavirus nsP2, an alphavirus nsP3, or an alphavirus nsP4. In some embodiments, the plurality of AAM RNAs further comprises a poly-adenosine monophosphate (poly(A)) tail.

In some embodiments, the plurality of AAM RNAs further comprises a 5′ untranslated region (5′ UTR), the 5′ UTR is 5′ of the first RNA or the second RNA. In some embodiments, the plurality of AAM RNAs further comprises a 3′ untranslated region (3′ UTR), the 3′ UTR is 3′ of the first RNA or the second RNA, and optionally is 5′ of the poly(A) tail. In some embodiments, the heterologous nucleic acid encoding a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody, an antibody against the immunogen, an immunotherapeutic molecule, or an antibody against an immune signaling molecule. In some embodiments, the heterologous protein comprises an immunogen or an antibody against the immunogen.

In some embodiments, the plurality of AAM RNAs further comprises a 5′ cap. In some embodiments, the 5′ cap is a cap-0, a cap-1, or a cap-2. In some embodiments, the 5′ cap is a cap-1. In some embodiments, the 5′ cap is a cap-0.

In one aspect, a composition is provided; the composition comprising any of the above-noted pluralities of AAM RNAs and a pharmaceutically acceptable delivery vehicle. In some embodiments, the delivery vehicle comprises a lipid nanoparticle (LNP). In some embodiments, the LNP encapsulates the plurality of AAM RNAs.

### Pluralities of Auto-Amplifying Messenger (AAM) RNAs, Methods of Use, and Methods of Manufacture, Wherein the Mole Amounts of Substitution is Based on the Admixtures Used to Obtain the Pluralities

In one aspect, a plurality of AAM RNAs comprising N1-methylpseudouridines, uridines, a first RNA, and one or more second RNA is provided. In some embodiments, the first RNA comprises a heterologous nucleic acid. In some embodiments, the one or more second RNA encodes one or more proteins capable of replicating the plurality of AAM RNAs in an intracellular environment. In some embodiments, the plurality of AAM RNAs is produced by a method, which comprises one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a first mole percentage of the N1-methylpseudouridines to the total of the N1-methylpseudouridines and the uridines from 15% to 75%. In some embodiments, the one or more admixtures has a first mole proportion of the N1-methylpseudouridines to the total of the N1-methylpseudouridines and the uridines from 0.15 to 0.75. In some embodiments, the one or more admixtures has a first mole ratio of the N1-methylpseudouridines to the uridines from 15:85 to 75:25. In some embodiments, the admixing being under conditions wherein the RNA polymerase produces the SAM RNA from the one or more template nucleic acids. In some embodiments, the first mole percentage of the plurality is any one of the embodiments provided in the sections above to SAM RNA having said mole percentages. In some embodiments, the first mole proportion is any one of the embodiments provided in the section above. In some embodiments, the first mole ratio is any one of the embodiments provided in the section above. In some embodiments, the one or more admixing is under conditions wherein the RNA polymerase produces the plurality of AAM RNAs from the one or more template nucleic acid.

In some embodiments, the first RNA comprises the N1-methylpseudouridines and the uridines. In some embodiments, the one or more second RNA comprises the N1-methylpseudouridines and the uridines. In some embodiments, the first mole percentage is to 70%. In some embodiments, the first mole percentage is to 65%. In some embodiments, the first mole percentage is to 60%. In some embodiments, the first mole percentage is to 55%. In some embodiments, the first mole percentage is to 50%. In some embodiments, the first mole percentage is from 20%. In some embodiments, the first mole percentage is from 25%.

In some embodiments, the one or more proteins capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments, the one or more proteins capable of replicating the plurality of AAM RNAs in an intracellular environment comprises an alphavirus non-structural protein-1 (nsP1), an alphavirus nsP2, an alphavirus nsP3, or an alphavirus nsP4.

In some embodiments, the heterologous nucleic acid encodes a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody, an antibody against the immunogen, an immunotherapeutic molecule, or an antibody against an immune signaling molecule. In some embodiments, the heterologous protein comprises an immunogen or an antibody against the immunogen. In some embodiments, the plurality of AAM RNAs further comprises a poly-adenosine monophosphate (poly(A)) tail. In some embodiments, the plurality of AAM RNAs further comprises a 5′ untranslated region (5′ UTR). In some embodiments, the 5′ UTR is 5′ of the first RNA or the second RNA. In some embodiments, the plurality of AAM RNAs further comprises a 3′ untranslated region (3′ UTR). In some embodiments, the 3′ UTR is 3′ of the first RNA or the second RNA, and optionally is 5′ of the poly(A) tail.

In some embodiments, the plurality of AAM RNAs further comprises a 5′ cap. In some embodiments, the 5′ cap is a cap-0, a cap-1, or a cap-2.

In one aspect, a composition is provided; the composition comprising any of the above-noted pluralities of AAM RNAs and a pharmaceutically acceptable delivery vehicle. In some embodiments, the pharmaceutically acceptable delivery vehicle comprises a lipid nanoparticle (LNP). In some embodiments, the LNP encapsulates the plurality of AAM RNAs.

In one aspect, a method of eliciting an immune response in a subject to the immunogen is provided; the method comprising administering to the subject an effective amount of any of the above-noted pluralities of AAM RNAs or any of the above-noted compositions. In some embodiments, the immune response is a protective immune response. In some embodiments, the immune response is a therapeutic immune response. In some embodiments, the immunogen comprises a venom, a poison, an allergen, a cancer antigen, a bacterial antigen, a viral antigen, a fungal antigen, a parasite antigen, or a fragment thereof. In some embodiments of any of the above-noted methods, the heterologous protein comprises the antibody against the immunogen. In some embodiments, the heterologous protein comprises the immunogen.

In one aspect, a method of delivering an inhibitory RNA in any of the above-noted pluralities of AAM RNAs to a subject is provided; the method comprising administering to the subject an effective amount of the plurality of AAM RNAs; the first RNA comprising an inhibitory RNA.

In one aspect, a method of delivering to a subject the inhibitory RNA in any of the above-noted compositions is provided; the method comprising administering to the subject an effective amount of the composition; the first RNA comprising an inhibitory RNA.

In some embodiments, the inhibitory RNA is against a endogenous messenger RNA encoding a endogenous protein of the subject, and the administering decreasing the expression of the endogenous protein in the subject when compared to the expression of the endogenous protein in the subject without the administering.

In one aspect, a method of delivering to a subject the heterologous nucleic acid in any of the above-noted pluralities of AAM RNAs is provided; the method comprising administering an effective amount of the plurality of AAM RNAs.

In another aspect, a method of delivering to a subject the heterologous nucleic acid in any of the above-noted compositions is provided; the method comprises administering to the subject an effective amount of the composition.

In one aspect, a method of manufacturing any of the above-noted compositions is provided; the method comprising encapsulating the plurality of AAM RNAs in the pharmaceutically acceptable delivery vehicle or adsorbing the plurality of AAM RNAs to the pharmaceutically acceptable delivery vehicle.

In another aspect, a use of any of the above-noted pluralities of AAM RNAs for the manufacture of a medicament for delivering the heterologous nucleic acid is provided; the use comprising admixing the plurality of AAM RNAs with a pharmaceutically acceptable delivery vehicle.

In another aspect, a use of any of the above-noted pluralities of AAM RNAs for the manufacture of a medicament for preventing a disease caused by a pathogen is provided; the pathogen comprising the immunogen; the use comprising admixing the plurality of AAM RNAs with a pharmaceutically acceptable delivery vehicle.

In another aspect, a use of any of the above-noted pluralities of AAM RNAs for the manufacture of a medicament for treating a disease caused by a pathogen is provided; the pathogen comprising the immunogen; the use comprising admixing the plurality of AAM RNAs with a pharmaceutically acceptable delivery vehicle.

In some embodiments, the pharmaceutically acceptable delivery vehicle comprises a lipid nanoparticle (LNP). In some embodiments, the LNP encapsulates the plurality of AAM RNAs.

In some embodiments, the immunogen comprises a venom, a poison, an allergen, a cancer antigen, a bacterial antigen, a viral antigen, a fungal antigen, a parasite antigen, or a fragment thereof. In some embodiments, the heterologous protein comprises the antibody against the immunogen. In some embodiments, the heterologous protein comprises the immunogen.

In one aspect, a plurality of AAM RNAs for use in eliciting an immune response to an antigen in a subject is provided; the plurality of AAM RNAs comprises N1-methylpseudouridines, uridines, a first RNA, and one or more second RNA. In some embodiments, the first RNA encodes the antigen. In some embodiments, the one or more second RNA encodes one or more proteins capable of replicating the plurality of AAM RNAs in an intracellular environment. In some embodiments, the plurality of AAM RNAs is produced by a method, which comprises one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a mole percentage of N1-methylpseudouridines to the total of the N1-methylpseudouridines and the uridines from 15% to 75%. In some embodiments, the one or more admixing is under conditions wherein the RNA polymerase produces the plurality of AAM RNAs from the template nucleic acid.

In one aspect, a plurality of AAM RNAs for use in preventing infection by a pathogen in a subject is provided; the pathogen produces an antigen; the plurality of AAM RNAs comprises N1-methylpseudouridines, uridines, a first RNA, and one or more second RNA. In some embodiments, the first RNA encodes the antigen. In some embodiments, the one or more second RNA encodes one or more proteins capable of replicating the plurality of AAM RNAs in an intracellular environment. In some embodiments, the plurality of AAM RNAs is produced by a method, which comprises one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a mole percentage of N1-methylpseudouridines to the total of the N1-methylpseudouridines and the uridines from 15% to 75%. In some embodiments, the one or more admixing is under conditions wherein the RNA polymerase produces the plurality of AAM RNAs from the template nucleic acid.

In one aspect, a plurality of AAM RNAs for use in treating infection by a pathogen in a subject is provided; the pathogen producing an antigen is provided; the plurality of AAM RNAs comprises N1-methylpseudouridines, uridines, a first RNA, and one or more second RNA. In some embodiments, the first RNA encodes the antigen. In some embodiments, the one or more second RNA encodes one or more proteins capable of replicating the plurality of AAM RNAs in an intracellular environment. In some embodiments, the plurality of AAM RNAs is produced by a method, which comprises one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a mole percentage of N1-methylpseudouridines to the total of the N1-methylpseudouridines and the uridines from 15% to 75%. In some embodiments, the one or more admixing is under conditions wherein the RNA polymerase produces the plurality of AAM RNAs from the template nucleic acid.

In some embodiments of any of the above-noted pluralities of AAM RNAs, the first RNA comprises the N1-methylpseudouridines and the uridines. In some embodiments of any of the above-noted pluralities of AAM RNAs, the one or more second RNA comprises the N1-methylpseudouridines and the uridines. In some embodiments of any of the above-noted pluralities of AAM RNAs, the subject is a human. In some embodiments of any of the above-noted pluralities of AAM RNAs, the first mole percentage is to 70%. In some embodiments of any of the above-noted pluralities of AAM RNAs, the first mole percentage is to 65%. In some embodiments of any of the above-noted pluralities of AAM RNAs, the first mole percentage is to 60%. In some embodiments of any of the above-noted pluralities of AAM RNAs, the first mole percentage is to 55%. In some embodiments of any of the above-noted pluralities of AAM RNAs, the first mole percentage is to 50%. In some embodiments of any of the above-noted pluralities of AAM RNAs, the first mole percentage is from 20%. In some embodiments of any of the above-noted pluralities of AAM RNAs, the first mole percentage is from 25%.

In some embodiments of any of the above-noted pluralities of AAM RNAs, the one or more proteins capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments of any of the above-noted pluralities of AAM RNAs, the one or more proteins capable of replicating the plurality of AAM RNAs in an intracellular environment comprises an alphavirus non-structural protein-1 (nsP1), an alphavirus nsP2, an alphavirus nsP3, or an alphavirus

In some embodiments, any of the above-noted pluralities of AAM RNAs further comprises a poly-adenosine monophosphate (poly(A)) tail. In some embodiments, any of the above-noted pluralities of AAM RNAs further comprises a 5′ untranslated region (5′ UTR). In some embodiments, the 5′ UTR is 5′ of the first RNA or the second RNA. In some embodiments, any of the above-noted pluralities of AAM RNAs further comprises a 3′ untranslated region (3′ UTR), the 3′ UTR is 3′ of the first RNA or the second RNA, and optionally is 5′ of the poly(A) tail.

In some embodiments, any of the above-noted pluralities of AAM RNAs further comprises a 5′ cap. In some embodiments, the 5′ cap is a cap-0, a cap-1, or a cap-2.

In some embodiments, the RNA polymerase is a T7 RNA polymerase.

In one aspect, a composition comprises any of the above-noted pluralities of AAM RNAs and a pharmaceutically acceptable delivery vehicle is provided. In some embodiments, the delivery vehicle comprises a lipid nanoparticle (LNP). In some embodiments, the LNP encapsulates the plurality of AAM RNAs.

In one aspect, a method of manufacturing any of the above-noted compositions is provided; the method comprises encapsulates the plurality of AAM RNAs in the pharmaceutically acceptable delivery vehicle or adsorbing the plurality of AAM RNAs to the pharmaceutically acceptable delivery vehicle.

In one aspect, a plurality of AAM RNAs for use in delivering a heterologous nucleic acid to a subject is provided; the plurality of AAM RNAs comprises N1-methylpseudorudines, uridines, a first RNA, and a second RNA. In some embodiments, the first RNA comprises a heterologous nucleic acid. In some embodiments, the second RNA encodes one or more proteins capable of replicating the plurality of AAM RNAs in an intracellular environment. In some embodiments, the plurality of AAM RNAs is produced by a method, which comprises one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs. In some embodiments, the one or more admixtures has a first mole percentage of N1-methylpseudouridines to the total of the N1-methylpseudouridines and the uridines from 15% to 75%. In some embodiments, the one or more admixtures has a first mole proportion of the N1-methylpseudouridines to the total of the N1-methylpseudouridines and the uridines from 0.15 to 0.75. In some embodiments, the one or more admixtures has a first mole ratio of the N1-methylpseudouridines to the uridines from 15:85 to 75:25. In some embodiments, the admixing being under conditions wherein the RNA polymerase produces the SAM RNA from the one or more template nucleic acids. In some embodiments, the first mole percentage of the plurality is any one of the embodiments provided in the sections above to SAM RNA having said mole percentages. In some embodiments, the first mole proportion is any one of the embodiments provided in the section above. In some embodiments, the first mole ratio is any one of the embodiments provided in the section above. In some embodiments, the one or more admixing is under conditions wherein the RNA polymerase produces the plurality of AAM RNAs from the one or more template nucleic acids.

In some embodiments, the subject is a human. In some embodiments, the first mole percentage is to 70%. In some embodiments, the first mole percentage is to 65%. In some embodiments, the first mole percentage is to 60%. In some embodiments, the first mole percentage is to 55%. In some embodiments, the first mole percentage is to 50%. In some embodiments, the first mole percentage is from 20%. In some embodiments, the first mole percentage is from 25%.

In some embodiments, the one or more proteins capable of replicating the plurality of AAM RNAs in an intracellular environment comprises an alphavirus non-structural protein-1 (nsP1), an alphavirus nsP2, an alphavirus nsP3, and an alphavirus nsP4. In some embodiments, the one or more proteins capable of replicating the plurality of AAM RNAs in an intracellular environment comprises an alphavirus non-structural protein-1 (nsP1), an alphavirus nsP2, an alphavirus nsP3, or an alphavirus nsP4. In some embodiments, any of the above-noted pluralities of AAM RNAs further comprises a poly-adenosine monophosphate (poly(A)) tail. In some embodiments, any of the above-noted pluralities of AAM RNAs further comprises a 5′ untranslated region (5′ UTR). In some embodiments, the 5′ UTR is 5′ of the first RNA or the one or more second RNA. In some embodiments, any of the above-noted pluralities of AAM RNAs further comprises a 3′ untranslated region (3′ UTR). In some embodiments, the 3′ UTR is 3′ of the first RNA or the second RNA, and optionally is 5′ of the poly(A) tail.

In some embodiments, the heterologous nucleic acid encoding a heterologous protein. In some embodiments, the heterologous nucleic acid comprises an inhibitory RNA. In some embodiments, the inhibitory RNA comprises an antisense RNA, a small interfering RNA, or a microRNA. In some embodiments, the heterologous protein comprises an immunogen, an antibody against the immunogen, an immunotherapeutic molecule, or an antibody against an immune signaling molecule. In some embodiments, the heterologous protein comprises an immunogen or an antibody against the immunogen.

In some embodiments, any of the above-noted pluralities of AAM RNAs further comprises a 5′ cap. In some embodiments, the 5′ cap is a cap-0, a cap-1, or a cap-2.

In one aspect, a composition is provided; the composition comprises any of the above-noted pluralities of AAM RNAs and a pharmaceutically acceptable delivery vehicle. In some embodiments, the delivery vehicle comprises a lipid nanoparticle (LNP). In some embodiments, the LNP encapsulates the plurality of AAM RNAs. [0134] Processes for the manufacture of self-replicating RNA are provided herein. In some embodiments, the process of manufacturing a self-replicating RNA comprises a step of in vitro transcription (IVT) as described elsewhere herein. In some embodiments, the process of manufacturing a self-replicating RNA comprises a step of IVT to produce a RNA, and further comprises a step of combining the RNA with a non-viral delivery system as described elsewhere herein. In some embodiments, the process of manufacturing a self-replicating RNA comprises a step of IVT to produce a RNA, and further comprises a step of combining the RNA with a CNE delivery system as described elsewhere herein.

### Sequence Identity

Identity or homology with respect to an amino acid sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity. Identity or homology with respect to a nucleic acid sequence is defined herein as the percentage of nucleotides in the candidate sequence that are identical with the reference nucleic acid sequence after aligning the sequences and introducing gaps, if necessary, to achieve the maximum percent sequence identity.

Sequence identity can be determined by standard methods that are commonly used to compare the similarity in position of the amino acids of two polypeptides. Using a computer program such as BLAST, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences. The same methods used to compare polypeptides can also be used to calculate the percent identity of two polynucleotide sequences.

Where the present disclosure refers to a sequence by reference to a UniProt or Genbank accession code, the sequence referred to is the current version at the filing date of the present application.

### EMBODIMENTS

Listing of numbered embodiments:
1. A self-amplifying messenger (SAM) ribonucleic acid (RNA) comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.
2. A SAM RNA comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.
3. A plurality of auto-amplifying messenger (AAM) RNAs comprising N1-methylpseudouridines, uridines, a first RNA, and one or more second RNA; the first RNA comprising a heterologous nucleic acid; the one or more second RNA encoding one or more proteins capable of replicating the AAM RNAs in an intracellular environment; the plurality of AAM RNAs having a first mole percentage of the N1-methylpseudouridines to the total of the N1-methypseudouridines and uridines from 15% to 75%.
4. A plurality of AAM RNAs comprising N1-methylpseudouridines, uridines, a first RNA, and one or more second RNA; the first RNA comprising a heterologous nucleic acid; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.
5. The SAM RNA of number 1 or number 2 or the plurality of AAM RNAs of number 3 or number 4, the first mole percentage being to 70%.
6. The SAM RNA of number 1 or number 2 or the plurality of AAM RNAs of number 3 or number 4, the first mole percentage being to 65%.
7. The SAM RNA of number 1 or number 2 or the plurality of AAM RNAs of number 3 or number 4, the first mole percentage being to 60%.
8. The SAM RNA of number 1 or number 2 or the plurality of AAM RNAs of number 3 or number 4, the first mole percentage being to 55%.
9. The SAM RNA of number 1 or number 2 or the plurality of AAM RNAs of number 3 or number 4, the first mole percentage being to 50%.
10. The SAM RNA of any one of numbers 1, 2, and 5-9 or the plurality of AAM RNAs of any one of numbers 3-9, the first mole percentage being from 20%.
11. The SAM RNA of any one of numbers 1, 2, and 5-9 or the plurality of AAM RNAs of any one of numbers 3-9, the first mole percentage being from 25%.
12. The SAM RNA of any one of numbers 1, 2, and 5-11 or the plurality of AAM RNAs of any one of numbers 3-11, the one or more proteins capable of replicating the SAM RNA in an intracellular environment comprising an alphavirus non-structural protein-1 (nsP1), an alphavirus nsP2, an alphavirus nsP3, and an alphavirus nsP4.
13. The SAM RNA of any one of numbers 1, 2, and 5-12 or the plurality of AAM RNAs of any one of numbers 3-12, the heterologous nucleic acid encoding a heterologous protein.
14. The SAM RNA of any one of numbers 1, 2, and 5-13 or the plurality of AAM RNAs of any one of numbers 3-13, the heterologous protein comprising an immunogen, an antibody, or an immunotherapeutic molecule.
15. The SAM RNA of any one of numbers 1, 2, and 5-13 or the plurality of AAM RNAs of any one of numbers 3-13, the heterologous protein comprising an immunogen or an antibody against the immunogen.
16. The SAM RNA of any one of numbers 1, 2, and 5-15 or the plurality of AAM RNAs of any one of numbers 3-16 further comprising a poly-adenosine monophosphate (poly(A)) tail.
17. The SAM RNA of any one of numbers 1, 2, and 5-16 or the plurality of AAM RNAs of any one of numbers 3-16 further comprising a 5′ untranslated region (5′ UTR), the 5′ UTR being 5′ of the first RNA segment and the second RNA segment.
18. The SAM RNA of any one of numbers 1, 2, and 5-17 or the plurality of AAM RNAs of any one of numbers 3-17 further comprising a 3′ untranslated region (3′ UTR), the 3′ UTR being 3′ of the first RNA segment and the second RNA segment, and optionally being 5′ of the poly(A) tail.
19. The SAM RNA of any one of numbers 1, 2, and 5-18 or the plurality of AAM RNAs of any one of numbers 3-18 further comprising a 5′ cap.
20. The SAM RNA or the plurality of AAM RNAs of number 19, the 5′ cap being a cap-0, a cap-1, or a cap-2.
21. The SAM RNA or the plurality of AAM RNAs of number 19, the 5′ cap being a cap-1.
22. The SAM RNA or the plurality of AAM RNAs of number 19, the 5′ cap being a cap-0.
23. A composition comprising the SAM RNA of any one of numbers 1, 2, and 5-22 or the plurality of AAM RNAs of any one of numbers 3-22 and a pharmaceutically acceptable delivery vehicle.
24. The composition of number 23, the pharmaceutically acceptable delivery vehicle comprising a lipid nanoparticle (LNP).
25. The composition of number 24, the LNP encapsulating the SAM RNA.
26. A method of eliciting an immune response in a subject to the immunogen, the method comprising administering to the subject an effective amount of the SAM RNA of any one of numbers 15-22 or the composition of any one of numbers 23-25.
27. The method of number 26, the immune response being a protective immune response.
28. The method of number 26, the immune response being a therapeutic immune response.
29. The method of any one of numbers 26-28, the immunogen comprising a venom, a poison, an allergen, a cancer antigen, a bacterial antigen, a viral antigen, a fungal antigen, a parasite antigen, or a fragment thereof.
30. The method of any one of numbers 26-29, the heterologous protein comprising the antibody against the immunogen.
31. The method of any one of numbers 26-29, the heterologous protein comprising the immunogen.
32. The method of any one of numbers 26-31, the subject being human.
33. A method of manufacturing the SAM RNA of any one of numbers 1 and 5-9, the method comprising admixing an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the admixing being under conditions wherein the RNA polymerase produces the SAM RNA from the template nucleic acid.
34. A method of manufacturing the SAM RNA of any one of numbers 19-22, the method comprising:
   a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the first admixing being under conditions wherein the RNA polymerase produces the SAM RNA from the template nucleic acid, thereby obtaining an uncapped SAM RNA; and a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.
35. The method of number 33 or number 34, the RNA polymerase being a T7 RNA polymerase.
36. A use of the SAM RNA of any one of numbers 15-22 or the plurality of AAM RNAs of any one of numbers 15-22 for the manufacture of a medicament for preventing a disease caused by a pathogen; the pathogen comprising the immunogen; the use comprising admixing the SAM RNA or the plurality of AAM RNAs with a pharmaceutically acceptable delivery vehicle.
37. The use of number 36, the pharmaceutically acceptable delivery vehicle comprising a lipid nanoparticle (LNP).
38. The use of number 37, the LNP encapsulating the SAM RNA or the plurality of AAM RNAs.
39. A SAM RNA for use in eliciting an immune response to an antigen in a subject; the SAM RNA comprising N1-methylpseudorudines, uridines, an RNA segment that encodes the antigen, and an RNA segment that encodes one or more proteins capable of replicating the SAM RNA in an intracellular environment; the SAM RNA having a first mole percentage of the N1-methylpseudouridines to the total of the N1-methylpseudouridines and the uridines from 15% to 75%.
40. A SAM RNA for use in eliciting an immune response to an antigen in a subject; the SAM RNA comprising N1-methylpseudouridines, uridines, a first RNA segment, and a second RNA segment; the first RNA segment comprising segment that encodes the antigen; the second RNA segment encoding one or more proteins capable of replicating the SAM RNA in an intracellular environment; the SAM RNA being produced by a method comprising admixing an RNA polymerase, the N1-methylpseudouridines, the uridines, and a template nucleic acid comprising a sequence of the SAM RNA, thereby obtaining an admixture; the admixture having a first mole percentage of the N1-methylpseudouridines to the total of the N1-methylpseudouridines and the uridines from 15% to 75%; the admixing being under conditions wherein the RNA polymerase produces the SAM RNA from the template nucleic acid.
41. A plurality of AAM RNAs for use in eliciting an immune response to an antigen in a subject, the plurality of AAM RNAs comprising N1-methylpseudouridines, uridines, a first RNA, and one or more second RNA; the first RNA comprising segment that encodes the antigen; the one or more second RNA encoding one or more proteins capable of replicating the AAM RNAs in an intracellular environment; the plurality of AAM RNAs having a first mole percentage of the N1-methylpseudouridines to the total of the N1-methypseudouridines and uridines from 15% to 75%.
42. A plurality of AAM RNAs for use in eliciting an immune response to an antigen in a subject, the plurality of AAM RNAs comprising N1-methylpseudouridines, uridines, a first RNA, and one or more second RNA; the first RNA comprising a segment that encodes the antigen; the one or more second RNA encoding one or more proteins capable of replicating the plurality of AAM RNAs in an intracellular environment; the plurality of AAM RNAs being produced by a method comprising one or more admixing of an RNA polymerase, the N1-methylpseudouridines, the uridines, and one or more template nucleic acids, thereby obtaining one or more admixtures wherein the one or more template nucleic acids comprise a sequence of the plurality of AAM RNAs; the one or more admixtures having a first mole percentage of N1-methylpseudouridines to the total of the N1-methylpseudouridines and the uridines from 15% to 75%; the one or more admixing being under conditions wherein the RNA polymerase produces the plurality of AAM RNAs from the one or more template nucleic acid.
43. A SAM RNA of number 39 or number 40 or the plurality of AAM RNAs of number 41 or number 42, the subject being a human.
44. The SAM RNA of any one of number 39, 40, and 43 or the plurality of AAM RNAs of any one of numbers 41-43, the first mole percentage being to 70%.
45. The SAM RNA of any one of number 39, 40, and 43 or the plurality of AAM RNAs of any one of numbers 41-43, the first mole percentage being to 65%.
46. The SAM RNA of any one of number 39, 40, and 43 or the plurality of AAM RNAs of any one of numbers 41-43, the first mole percentage being to 60%.
47. The SAM RNA of any one of number 39, 40, and 43 or the plurality of AAM RNAs of any one of numbers 41-43, the first mole percentage being to 55%.
48. The SAM RNA of any one of number 39, 40, and 43 or the plurality of AAM RNAs of any one of numbers 41-43, the first mole percentage being to 50%.
49. The SAM RNA of any one of number 39, 40, and 43-48 or the plurality of AAM RNAs of any one of numbers 41-48, the first mole percentage being from 20%.
50. The SAM RNA of any one of number 39, 40, and 43-48 or the plurality of AAM RNAs of any one of numbers 41-48, the first mole percentage being from 25%.

### EXAMPLES

### SAM RNA Synthesis—General Methods

Self-amplifying mRNAs were prepared as previously described, (Hekele et al Emerg Microbes Infect 2013; 2 doi:ARTN e52DOI 10.1038/emi.2013.54), except that for mole ratios of N1-methylpseudouridine triphosphate to uridine triphosphates were made at 15:85, 25:75, 50:50, 75:25, and 100:0 in the admixture of dNTPs used in the in vitro transcription reaction used to produce the SAM RNA. For convenience throughout the Examples and with regard to SAM RNA described as having a certain percentage of substitutions of uridines with N1-methylpseudouridines, said percentages of substitutions are understood to be based on the ratio of the N1-methylpseudouridine triphosphate to uridine triphosphates in the admixture of the in vitro transcription reaction.

Plasmids were amplified in Escherichia coli and purified using Qiagen maxi Kits (Qiagen, Valencia, Calif., USA). DNA was linearized immediately following the 3′ end of the self-amplifying RNA sequence by restriction digestion with Pmel. Linearized DNA plasmids were transcribed into RNA using MEGAscript T7 Kit (Life Technologies) and purified by LiCl precipitation. RNA was then capped using the ScriptCap m₇G capping system (Cell Script) and purified by 2.8 M LiCl precipitation before formulation. Protein expression was confirmed by Western blot analysis of transfected cell lysate.

The following are examples of the mass per volume yields of SAM RNA. The A1010 designation denotes that the SAM RNA encodes a firefly luciferase. The JW13 designation indicates a SAM RNA with an insertion in the nucleic acid encoding nsP1-4 preprotein, which thereby triggers an early stop codon that prevents translation of the full length nsP1-4 preprotein and cleavage of any of the individual proteins nsP1 through nsP4. The percentage indicates the mole percentage of uridine to N1-methylpseudouridine substitution.
IVTCAP0-A1010-N1Ψ0%-2 986 ng/ul
IVTCAP0-A1010-N1Ψ25%-2 1030 ng/ul
IVTCAP0-A1010-N1Ψ50%-2 960 ng/ul
IVTCAP0-A1010-N1Ψ100%-2 788 ng/ul
IVTCAP0-JW13-N1Ψ0%-2 836 ng/ul
IVTCAP0-JW13-N1Ψ100%-2 748 ng/ul
IVTCAP1-A1010-N1Ψ0%-2 1220 ng/ul
IVTCAP1-A1010-N1Ψ25%-2 1287 ng/ul
IVTCAP1-A1010-N1Ψ50%-2 1287 ng/ul
IVTCAP1-A1010-N1Ψ100%-2 1237 ng/ul
IVTCAP1-JW13-N1Ψ0%-2 1228 ng/ul
IVTCAP1-JW13-N1Ψ100%-2 1233 ng/ul

These SAM RNA were electroporated and stained with ethidium bromide to confirm their sizes and specificity of products.

### RNA Amplification and Protein Expression Analysis—General Methods

RNA amplification efficiency was carried out, as previously reported (Magini et al, PLoS One 2016; 11(8):e0161193). Briefly, baby hamster kidney cells (BHK cells), BJ cells, C2C12 cells, human skeletal muscle cells (hSkM), or human peripheral blood mononuclear cells (PBMC) were electroporated with the specified amounts of RNA and incubated for as long as is indicated at 37° C. and 5% CO₂. In some instances, the cells were assessed for the reporter in the SAM RNA (i.e., firefly luciferase or green fluorescent protein). In some instances, cells were collected and stained with Live/Dead Aqua (Invitrogen) or APC-conjugated anti-double-stranded (ds) RNA antibody (J2 monoclonal mouse antibody IgG2a kappa chain, Bioclass). Frequencies of dsRNA+cells were analyzed by FACS CANTO II flow cytometer (BD Biosciences).

To confirm expression of proteins from SAM RNA, 10⁶ BHK, BJ, C2C12, hSkM, or PBMC cells were transfected with RNA using Lipofectamine 2000™ (LifeTechnologies, Calif., USA). Cells were harvested 16 hours after transfection and lysed in 100 μL of 1× radio-immunoprecipitation assay (RIPA) buffer (Cell Signaling Technology, Mass., USA) with complete protease inhibitor cocktail (Roche, Madison, Wis., USA). Lysates were subjected to SDS-PAGE and blotted on to PVDF membrane. Protein expression was detected with the indicated monoclonal or polyclonal antibodies.

### LNP/RNA Formulation—General Methods

Equal amount of RNAs were mixed prior to encapsulation in LNPs. Formulations were characterized for particle size, RNA concentration, encapsulation efficiency and RNA integrity (using gel electrophoresis) as previously described (Hekele et al, Emerg Microbes Infect 2013). Encapsulation of RNA within LNP was carried out as described previously (Geall et al Proc Natl Acad Sci USA 2012; 109:14604-9). DLinDMA was synthesized as previously described (Heyes et al, J Control Release 107:276-287). The 1,2-Diastearoyl-sn-glycero-3-phosphocholine (DSPC) was purchased from Genzyme. Cholesterol was obtained from Sigma-Aldrich. 1,2-dimyristoyl-sn-glycero-3-phosphoethanolamine-N-[methoxy(polyethylene glycol)-2000] (ammonium salt) (PEG DMG 2000) was obtained from Avanti Polar Lipids. An ethanol dilution process was used to produce the LNP formulation with the following molar ratios of lipid components: DSPC: cholesterol: PEG-DMG 2000: DLinDMA 10:48:2:40 molar percent. An 8:1 N:P molar ratio (nitrogen on DlinDMA to phosphate on RNA) and 100 mM citrate buffer (pH 6) were used for the formulations. In the first step of the in-line mixing, equal volumes of lipid (in ethanol) and RNA in buffer were mixed, through a T-junction via a KDS-220 syringe pump (kdScientific), and a third syringe with equal volume of buffer was added simultaneously to the lipid/RNA mixture. After 1 h equilibration at room temperature, the mixture was further diluted with 1:1 vol/vol citrate buffer. Next, the LNPs obtained (“RV01” LNPs) were concentrated and dialyzed against 1× PBS using tangential flow filtration (TFF) (Spectrum Labs) with polyethersulfone (PES) hollow fiber membranes with a 100-kDa pore size cutoff and 20 cm.sup.2 surface area. For in vitro and in vivo experiments, formulations were diluted to the required RNA concentration with 1′ PBS (Teknova).

### Statistical Analyses—General Methods

All statistical analyses were performed using GraphPad Prism 5 software (GraphPad Software, La Jolla, USA). Mann-Whitney U test was used to analyze HI titers and T-cell frequencies. P value of less than 0.05 was considered significant.

### In Vitro Examples

### Example 1

Cells, 10⁶ C2C12 cells, were transfected with 100 ng of SAM RNA as indicated below and 18 hours later were assessed for firefly luciferase and/or double-stranded RNA formation by J2-APC immunocytochemistry followed by FACS or for IFN-β levels by ELISA.

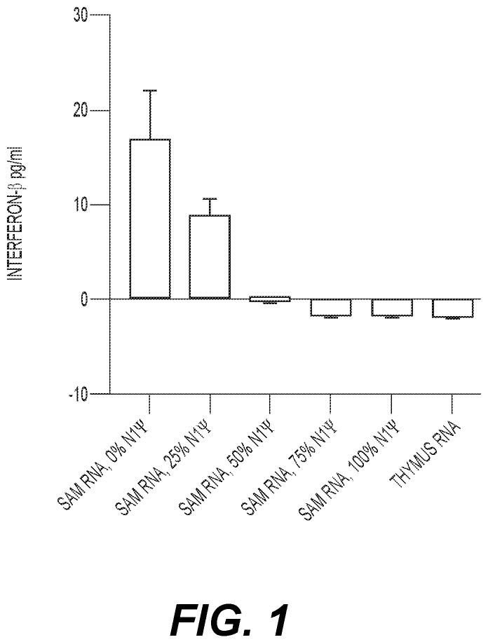

FIG. 1 shows interferon-β (IFN-β) levels from C2C12 cells electroporated with 100 ng of self-amplifying messenger (SAM) ribonucleic acid (RNA) wherein 0%, 25%, 50%, 75%, or 100% of the uridines (U) are substituted with N1-methylpseudouridine (N1Ψ). Levels of IFN-β decrease in C2C12 cells administered of a SAM RNA comprising 25% substitution when compared to the levels from the same cells administered SAM RNA comprising 0% substitution. Levels further decrease with administration of SAM RNA comprising 50%, 75%, or 100% substitution compared to those with administration of SAM RNA comprising 25% substitution.

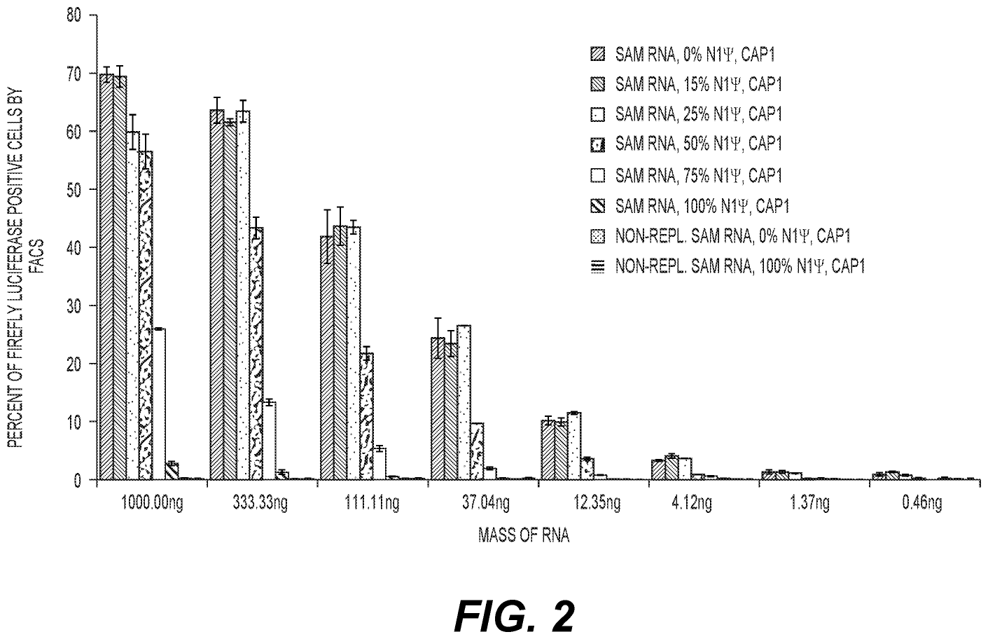

FIG. 2 depicts the percentage, as identified with fluorescence-activated cell sorting (FACS), of C2C12 cells expressing firefly luciferase encoded by SAM RNA comprising 0%, 15%, 25%, 50%, 75%, or 100% of substitution of U to N1Ψ, which was administered in a mass of 1000 ng, 333.33 ng, 111.11 ng, 37.04 ng, 12.35 ng, 4.12 ng, 1.37 ng, or 0.46 ng. The percentage of C2C12 cells expressing firefly luciferase slightly decreases with 50% substitution when compared to that with 0%, 15%, or 25% substitution and further decreases with 75% substitution when compared to that with 50% substitution. With 100% substitution, almost an undetectable percentage of cells express the firefly luciferase. When compared to the IFN-β levels shown in FIG. 1, the decreases in the expression of firefly luciferase with increasing substitution lags the decreases IFN-β levels with increasing substitution. That is, the IFN-β levels first decreases with 25% substitution, and the firefly luciferase expression first and slightly decreases with 50% substitution with 1000 ng of SAM RNA administered.

### Example 2

LEEPORTER™ luciferase reporter TLR7 and TLR8 cells were used according to the manufacturer's instructions.

In brief, for the LEEPORTER™ luciferase reporter cells:

Cells should be grown at 37° C. with 5% CO₂ using DMEM medium (w/L-Glutamine, 4.5 g/L Glucose and Sodium Pyruvate) supplemented with 10 volume % heat-inactivated FBS and 1% Pen/Strep, plus 2 μg/ml of Puromycin and 5 μg/ml of Blasticidin.

These cells were quickly thawed upon receipt in a 37° C. water-bath, transferred to a tube containing 10 mL of growth medium without Puromycin and Blasticidin, spun down, and resuspended in prewarmed growth medium without Puromycin and Blasticidin. Thereafter, they were transferred T25 flask and cultured in 37° CO₂ incubator for 1˜3 days without disturbing or changing the medium until cells completely recovered viability and became adherent. Once the cells were over 90% adherent, the growth medium was removed, and the cells were passaged through trypsinization and centrifugation. At first passage, the growth medium containing Puromycin and Blasticidin was switched. The cells were also split before they reached complete confluence. Never were the cells passaged more than 16 times.

To perform the assay, the TLR7/NF-kB or TLR8/NF-kB Leeporter™—HEK293 cells were harvested and seeded into a white solid-bottom 96-well microplate in 100 μL of growth medium at 5×10⁴ cells/well. The cells were then incubated at 37° C. in at 5% CO₂ overnight. The next day, the next day the cells were transfected with the SAM RNA/LNP, and as positive controls stimulated with R848, and then incubated at 37° C. in at 5% CO₂. They were then equilibrated to room temperature for 10 minutes and 50 μL of luciferase assay reagent (Abeomics, Cat #17-1101) was added per well. The plate was then read within 1-5 minutes to measure renilla luciferase luminescence using a microplate luminometer.

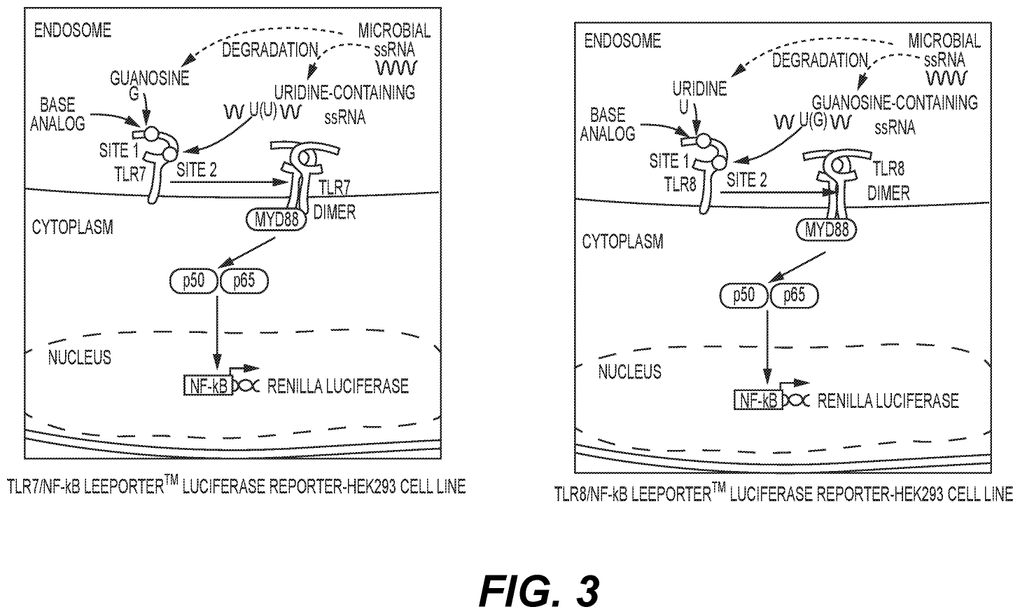

FIG. 3 depicts the intracellular signaling pathway of LEEPORTER™ luciferase reporter human embryonic kidney 293 (HEK 293) cells that starts with toll-like receptor 7 (TLR7; left) and toll-like receptor 8 (TLR8, right) activation by single-stranded ribonucleic acid (ssRNA), signals through myeloid differentiation primary response 88 (MyD88), activates p50 and p65 and then nuclear factor κ light chain enhancer of activated B cells (NF-κB), and results in renilla luciferase expression. The renilla luciferase signal is discrete from firefly luciferase signaling, which depending upon the assay is encoded by the ribonucleic acid (RNA) used to activate TLR7 or TLR8. www.abeomics.com/tlr7-nf-kb-leeporter-luciferase-reporter-hek293-cell-line; www.abeomics.com/tlr8-nf-kb-leeporter-luciferase-reporter-hek293-cell-line.

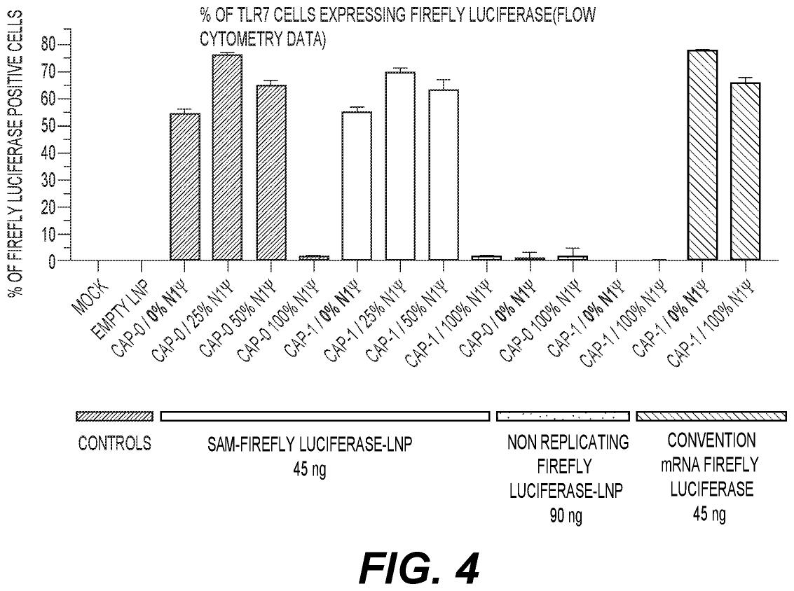

FIG. 4 shows the percent of TLR7 positive human embryonic kidney 293 (HEK293) cells (TLR7/NF-kB Leeporter™ Renilla Luciferase Reporter-HEK293 Cell Line) that express firefly luciferase upon transfection with self-amplifying messenger (SAM) riboxynucleic acid (RNA), SAM RNA deficient in the self-amplifying elements (non-replicating SAM RNA obtained with a mutation abolishing the replicase complex activity), or conventional RNA. The SAM RNA, non-replicating SAM RNA, and conventional RNA encode the firefly luciferase, have 0%, 25%, 50%, or 100% substitution of the U with N1Ψ, and are capped with cap 0 or cap 1. Increasing the amount of substitution from 0% to 25% or 50% increases the number of cells that express the firefly luciferase encoded by the exogenous SAM RNA with cap 0 or cap 1. The percent of firefly luciferase positive cells decreases with the 50% substitution compared to that with the 25% substitution. One hundred percent substitution in SAM RNA results in almost no detectable firefly luciferase positive cells regardless of whether the SAM RNA has cap 0 or cap 1, in contrast to conventional RNA that is 100% substituted.

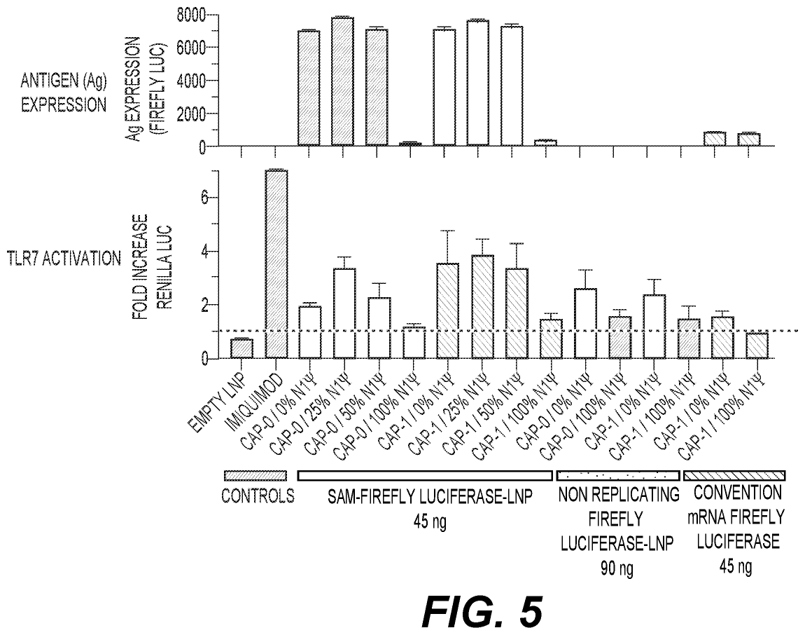

FIG. 5 depicts total antigen expression (top, firefly luciferase expression encoded by the RNA) and TLR7 activation (bottom, renilla luciferase expression downstream of TLR7, MyD88, p50, p65, and NF-κB) with the same SAM RNA, non-replicating SAM RNA, and conventional RNA as described above for FIG. 4. Twenty five percent substitution of U to N1Ψ elevated total antigen expression above levels with 0% substitution with cap 0 and cap 1 capping. The non-replicating SAM RNA comprises an insertion in nucleic acid encoding the non-structural protein 2 thereby causing a missense translation. Increasing the substitution from 0% to 100% in non-replicating SAM RNA decreases TLR7 activation regardless of capping, thereby demonstrating that with initial administration, the substitution evades the innate immune response. With the SAM RNA (self-replicating), 25% substitution increased TLR7 activation compared to activation with 0% substitution with cap 0 capping. In combination with the results with non-replicating SAM, this demonstrates that intracellularly the self-replication incorporates non-modified forms of uridine, and these strands activate TLR7.

### Example 3

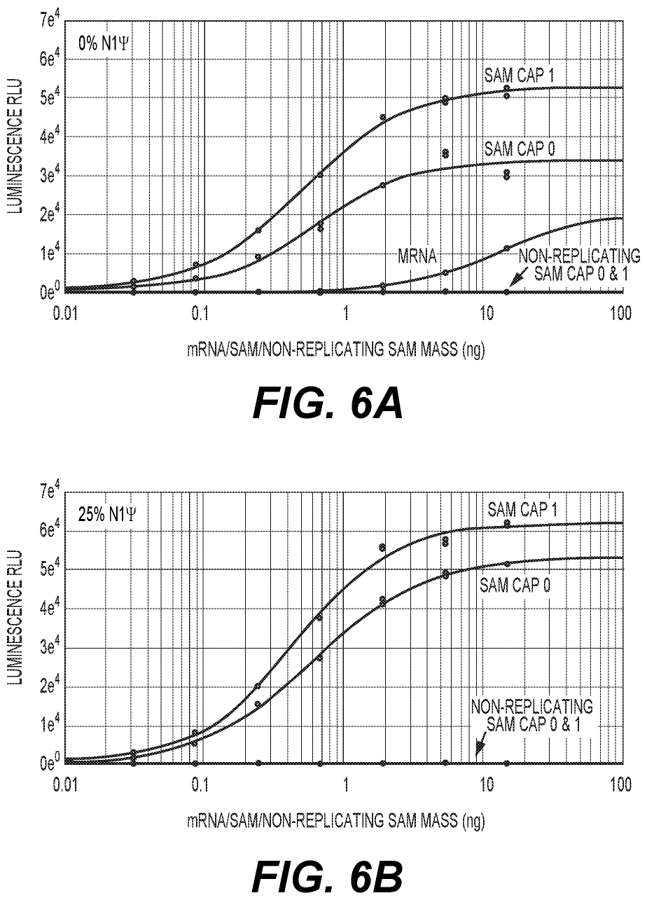

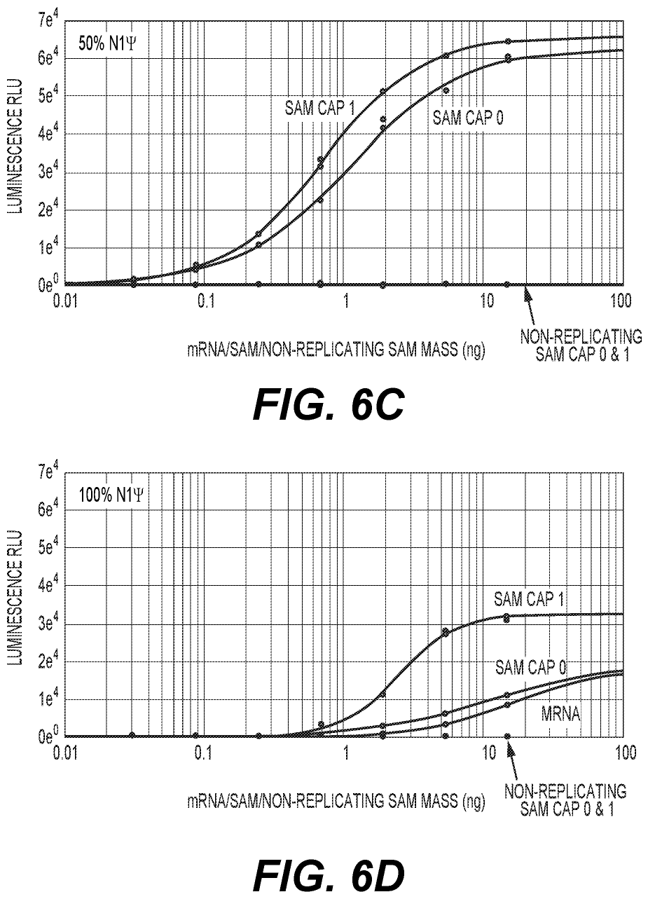

BJ cells were transfected as described above. Capping with cap 1 results in greater firefly luciferase luminescence in all conditions and a leftward shift in the dose response curve compared to that for cap 0 under all conditions of substitution with the SAM RNA. Antigen expression increases with 25% and 50% substitution of the SAM RNA, regardless of capping, compared that with 0% and 100% substitution of the SAM RNA or to that with conventional RNA. FIGS. 6A-6D depict dose response curves of luminescence of firefly luciferase in a polyploid human foreskin fibroblast cell line, the BJ cell line, with transfected with SAM RNA capped with cap 0 or cap 1, conventional RNA, or non-replicating SAM capped with cap 0 or cap 1. The SAM RNA, non-replicating SAM RNA, or conventional RNA encode firefly luciferase and U are 0% (FIG. 6A), 25% (FIG. 6B), 50% (FIG. 6C), or 100% (FIG. 6D) substituted with N1Ψ.

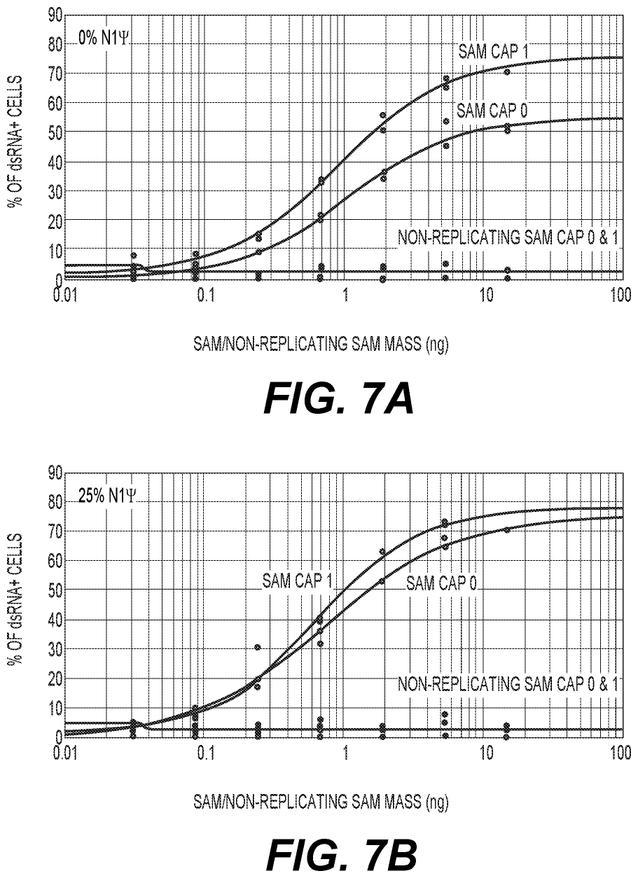

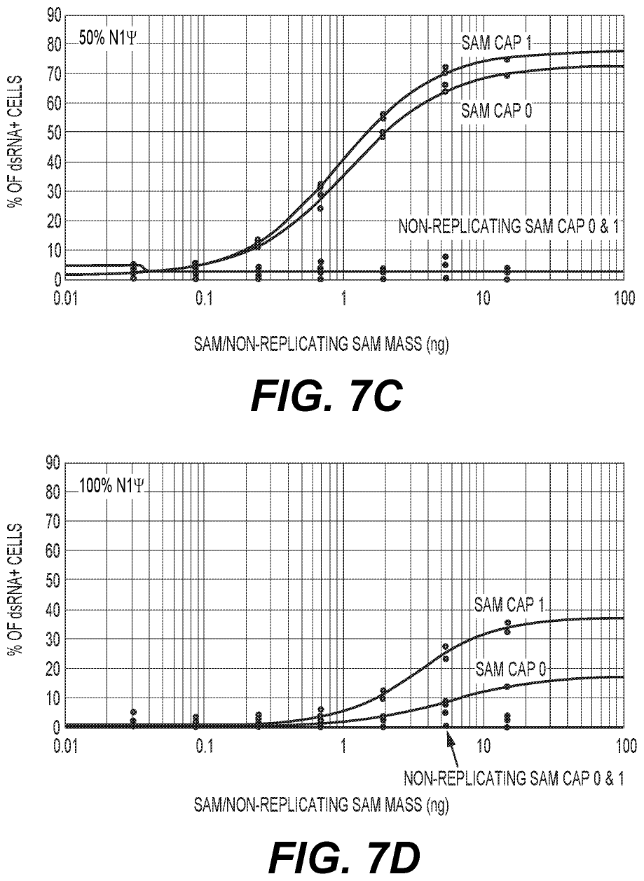

FIGS. 7A-7D show dose response curves of the percent of BJ cells expressing double-stranded RNA (% of dsRNA+ cells) that replicated from the SAM RNA capped with cap 0 or cap 1, conventional RNA, or non-replicating SAM capped with cap 0 or cap 1, wherein the U are 0% (FIG. 7A), 25% (FIG. 7B), 50% (FIG. 7C), or 100% (FIG. 7D) substituted with N1Ψ (0% N1Ψ, 25% N1Ψ, 50% N1Ψ, and 100% N1Ψ respectively). Capping with cap 1 results in a leftward shift in the dose response curve compared to that with cap 0 under all conditions of substitution with the SAM RNA. The percent of BJ cells expressing double-stranded RNA plateaued to about same level (75%-78%) with 0%, 25%, or 50% substitution of U with N1Ψ with cap 1 capping. This indicates that, regardless of the percent substitution, the cap 1 capping can obtain a maximum response with a maximal dose (excepting 100% substitution). Nonetheless, 25% substitution can have an effect with cap 1 capping on the percentage of cells when compared to that with 0% substitution. When comparing the 50% mark for the percent of cells containing double-stranded RNA, there is a leftward shift with 25% substitution compared to that with 0% or 50% substitution. With cap 0 capping, 25% and 50% substitution increased the maximum percentage of cells expressing double-stranded RNA compared to that with 0% or 100% substitution. Therefore, there is an effect of substitution of U to N1Ψ on the self-amplification of SAM RNA substitution, whereby 25% or 50% substitution can increase the number of cells with self-amplification compared to that with 0% substitution, and this effect occurs within cap 0 or cap 1 capping.

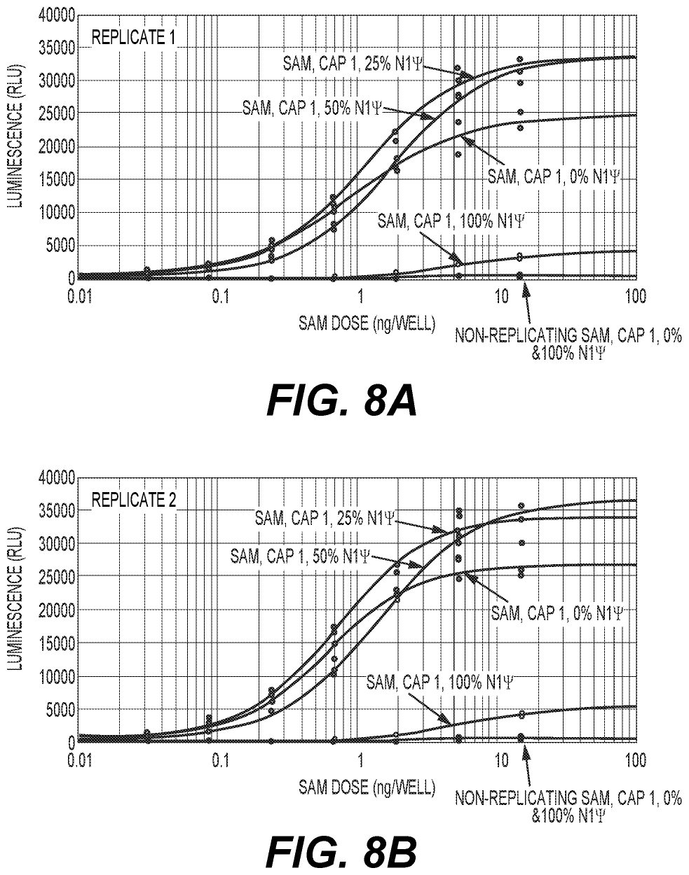

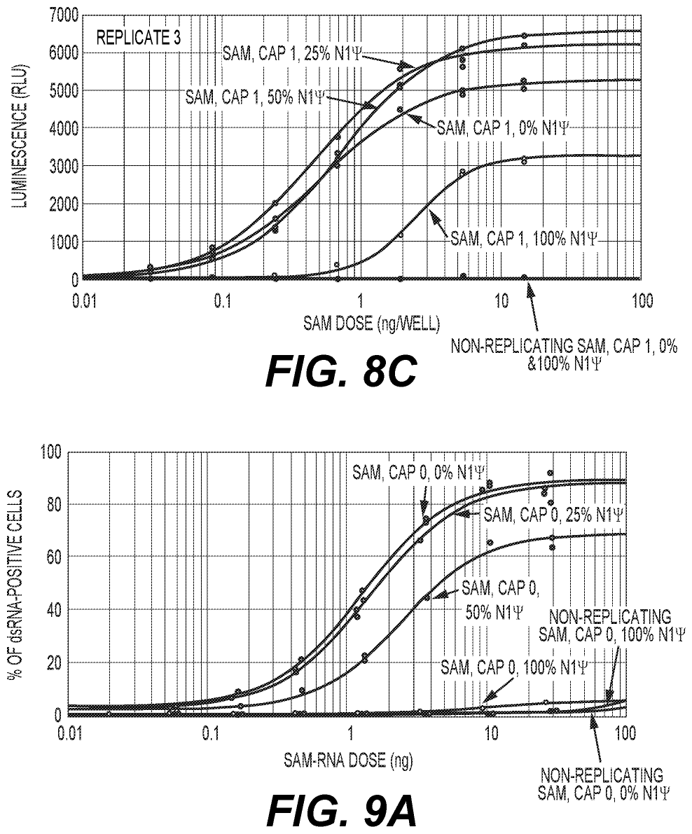

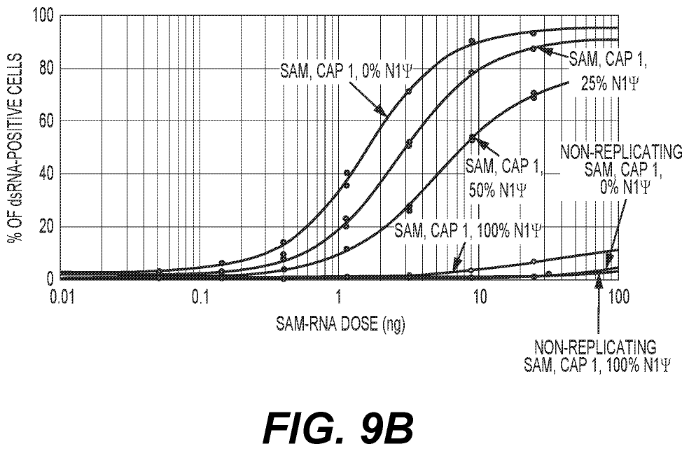

FIGS. 8A-8C shows three replicates of dose response curves of total luminescence from the RNA encoding firefly luciferase with SAM RNA with cap 1 and 0%, 25%, 50%, and 100% of U to N1Ψ substitution. In each replicate, there is a leftward shift with 25% substitution compared to 0% substitution and the total amount of luciferase expressed from the SAM RNA reaches a higher maximum with 25% and 50% substitution than the maximum with 0% substitution. These results, together with the results from FIGS. 7A-7D, demonstrate that 25% and 50% substitution increase expression of amino acids encoded by the SAM RNA compared to the same measures with 0% substitution. Even if as many cells express the amino acid encoded by the SAM RNA when there is 0% substitution as when there is 25% or 50% substitution, those cells obtain increased expression of the amino acid encoded by the SAM RNA. Whether this effect is due to evasion of the innate immune system with the 25% or 50% substitution is determined in FIGS. 9A and 9B.

### Example 4

Baby hamster kidney (BHK) cells were transfected with SAM RNA as described above.

BHK cells, which are deficient in innate sensing and activation, were selected to determine whether the 25% or 50% substitutions evaded the innate immune response (i.e., a TLR7-activated or TLR8-activated immune response) thereby increasing the percentage of cells having RNA that self-amplifies (FIGS. 7B and 7C) and the total amount of luminescence from the firefly luciferase (FIGS. 8A-8C) encoded by the SAM RNA when compared the same results with 0% or 100% substitution (FIGS. 7A, 7D, and 7A-7C).

FIGS. 9A and 9B depict dose response curves of the percent of baby hamster kidney (BHK) cells that express double-stranded RNA (% of dsRNA+ cells) replicating from the SAM RNA capped with cap 0 (FIG. 8A) or cap 1 (FIG. 8B), conventional RNA, or non-replicating SAM capped with cap 0 or cap 1, wherein the U in the RNA are 0%, 25%, 50%, or 100% substituted with N1Ψ. Increasing the substitution of U to N1Ψ from 0% to 25%, then to 50%, and then to 100% caused a rightward shift in the dose response curve regardless of whether cap 0 or cap 1 capped the SAM RNA. Therefore, evasion of the innate immune system increases the self-amplification of the SAM RNA, as measured by double-stranded RNA, and the expression of amino acids encoded by the SAM RNA observed in cells with innate immune responses with 25% or 50% substitution over the same results observed with 0% substitution. Because BHK cells are deficient in innate immune activation, these results cannot be used to infer that the improvements in self-amplification with 25% or 50% substitution are cell-specific or context-specific.

### Example 5

BJ cells were transfected with SAM RNA as described above and were assessed for firefly luciferase 24 hours later, wherein the SAM RNA encoded the firefly luciferase. Thereafter, the cells were trypsinized from the bottom of the plate and were sorted by FACS for firefly luciferase expression. In the alternative, the cells were assessed for IL-6 levels at the same timepoint, by obtaining supernatant from the 200 mL well plate.

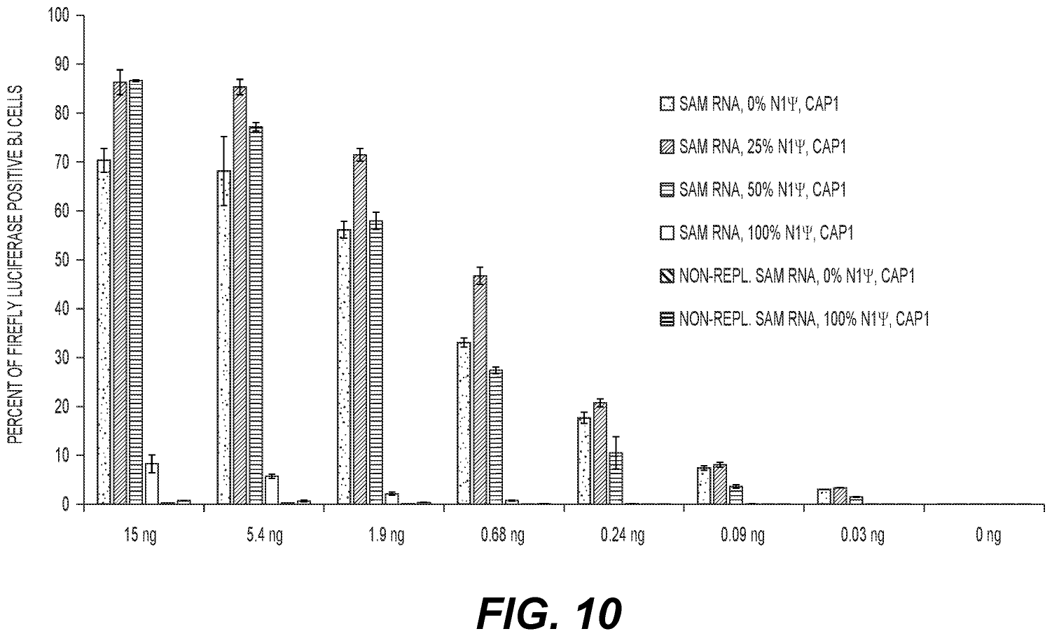

With administration of 15 ng, 5.4 ng, or 1.9 ng of replicating, 25% substituted SAM RNA, the percentage of firefly luciferase positive BJ cells increases over that with administration of replicating SAM RNA lacking substitution. With administration of 15 ng or 5.4 ng of replicating, 50% substituted SAM RNA, the percentage of firefly luciferase positive cells also increases compared to that with administration of replicating SAM RNA lacking substitution. FIG. 10 depicts the percentage, as identified with FACS, of BJ cells expressing firefly luciferase encoded by SAM RNA comprising 0%, 25%, 50%, or 100% of substitution of U to N14), which was administered in a mass of 15 ng, 5.4 ng, 1.9 ng, 0.68 ng, 0.24 ng, 0.09 ng, 0.03 ng, or 0 ng.

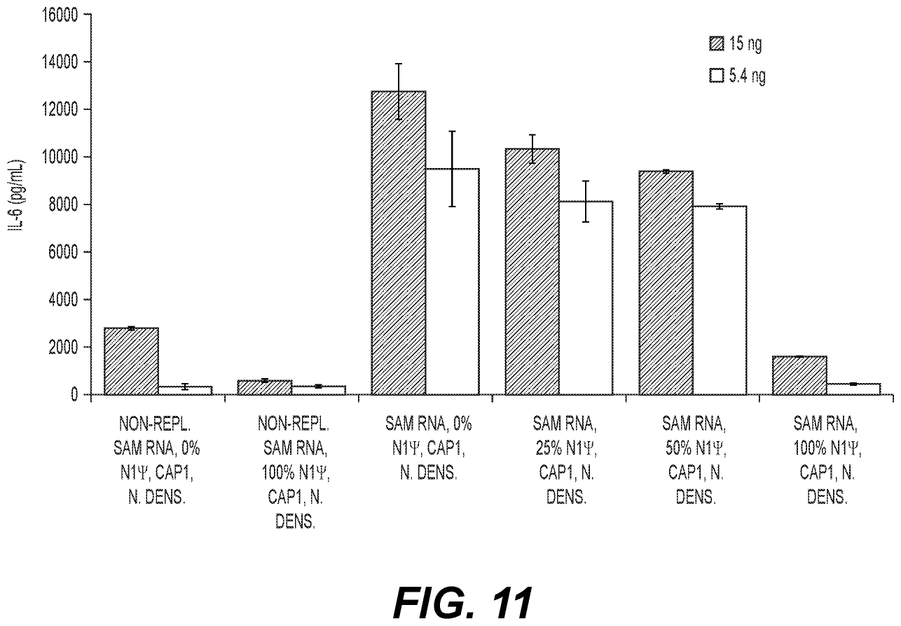

FIG. 11 shows interleukin 6 (IL-6) levels from BJ cells transfected with lipid nanoparticles encapsulating SAM RNA, non-replicating SAM RNA, or mRNA, wherein 0%, 25%, 50%, and 100% of U are substituted with N1Ψ and the RNA is capped with cap 1. Administration of non-replicating SAM RNA comprising 100% U to N1Ψ substitution reduces IL-6 expression compared to expression with administration of 0% substituted non-replicating SAM RNA. Therefore, increasing substitution decreases IL-6 levels. The non-replicating SAM induce lower IL-6 expression than expression with replicating SAM RNA. Self-replication of SAM RNA within a cell incorporates uridines into the newly synthesized strands, and thereby increases IL-6 levels regardless of the initial substitution. Within the self-replicating SAM RNA, increasing the substitution from 0% to 25%, and then to 50%, and then to 100% reduces levels of IL-6 with each increment of substitution.

### Example 6

The human peripheral blood assay (hPBMC) used a panel of independent normal human donors according to approved guidelines by the institutional review committee. Human PBMC were isolated from freshly peripheral blood using a Ficoll density gradient (GE healthcare 17-1440-03), 30-35 mLs of peripheral human blood were layered onto 15 mL of Ficoll in 50 mL conical tubes, followed by centrifugation at 1800 rpm (Eppendorf Centrifuge 581 OR with biohazard caps over the tube buckets) at room temperature for 30 minutes with no acceleration and no brake. The layers were then collected and transferred onto new 50 mL conical tubes and washed twice in complete media consisting of RPMI 1640 (11875085 from Invitrogen Corporation, Carlsbad, Calif.) supplemented with 10% heat inactivated fetal bovine serum (Gibco 10099-141), 1% Pen-Strep (Gibco #15140-122), 1 mM non-essential amino acids (Gibco #11140-050), 1 mM sodium pyruvate (Gibco #11360-070), 2 mM L-Glutamine (Gibco #25030-081) and 1 mM HEPES (Gibco #15630-080). Viable cells were then counted using trypan blue staining, plated in 96 well flat bottom plates (Becton Dickinson #353070) at 2×10⁵ cells per well in 200 μL total volume of complete media. The hPBMC cells were transfected as described above.

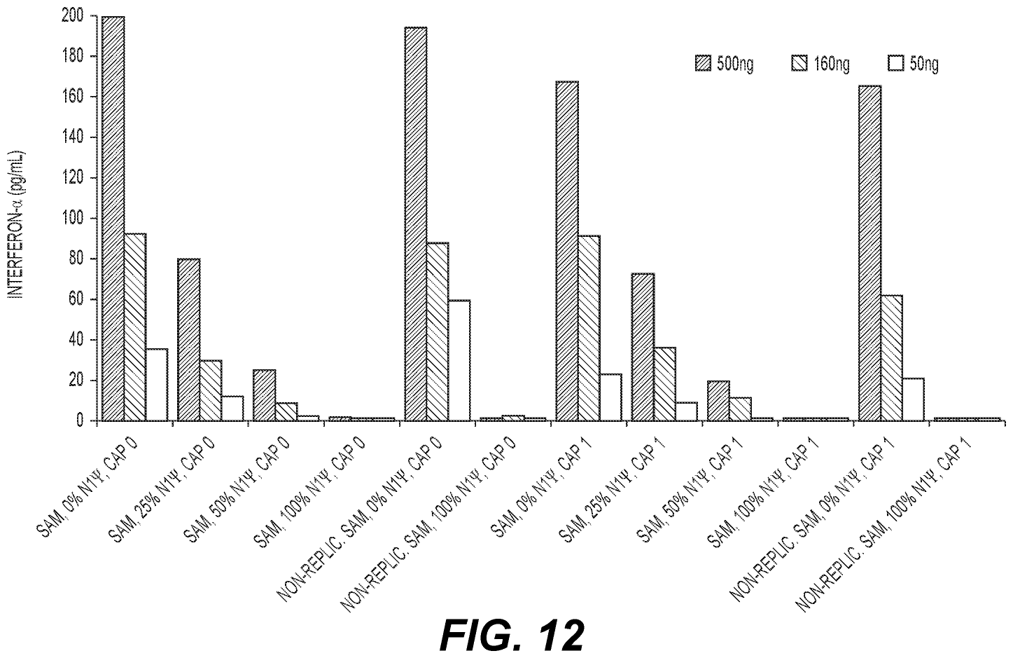

Increasing the substitution from 0% to 25%, and then to 50%, and then to 100% in the self-replicating and non-replicating SAM RNA reduces, at each increment of substitution, the levels of IFN-α. Cap 1 capping compared to cap 0 capping reduces IFN-α levels when the hPBMC cells were exposed to 500 ng of RNA. FIG. 12 depicts interferon-α (IFN-α) levels obtained from one donor of human peripheral blood monocyte cells (PBMCs) transfected with lipid nanoparticles encapsulating SAM RNA, non-replicating SAM RNA, wherein 0%, 25%, 50%, and 100% of U are substituted with N1Ψ and the RNA is capped with cap 0 or cap 1. This result was replicated with other individuals' PBMCs, but only one individual's PBMCs are shown as representative, and to compensate for baseline level differences across individuals' PBMCs.

### Example 7

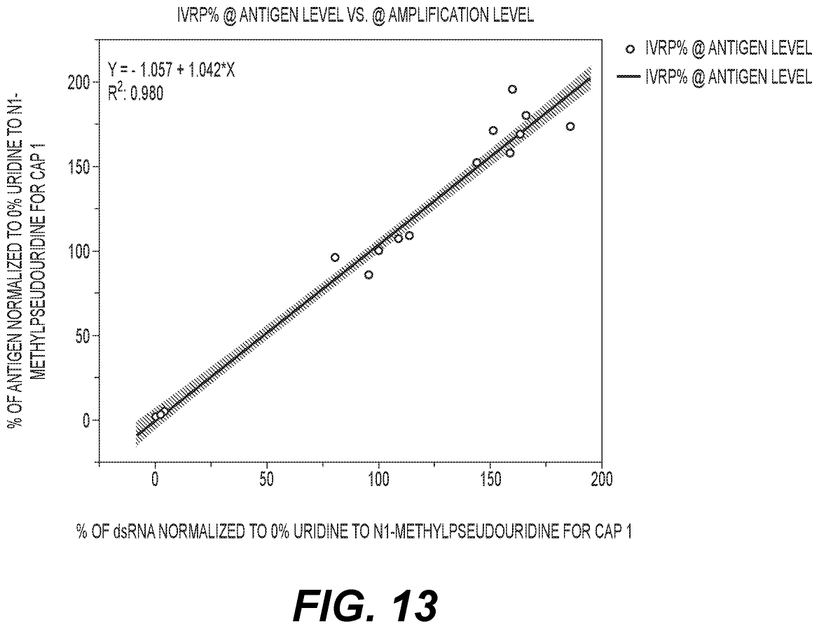

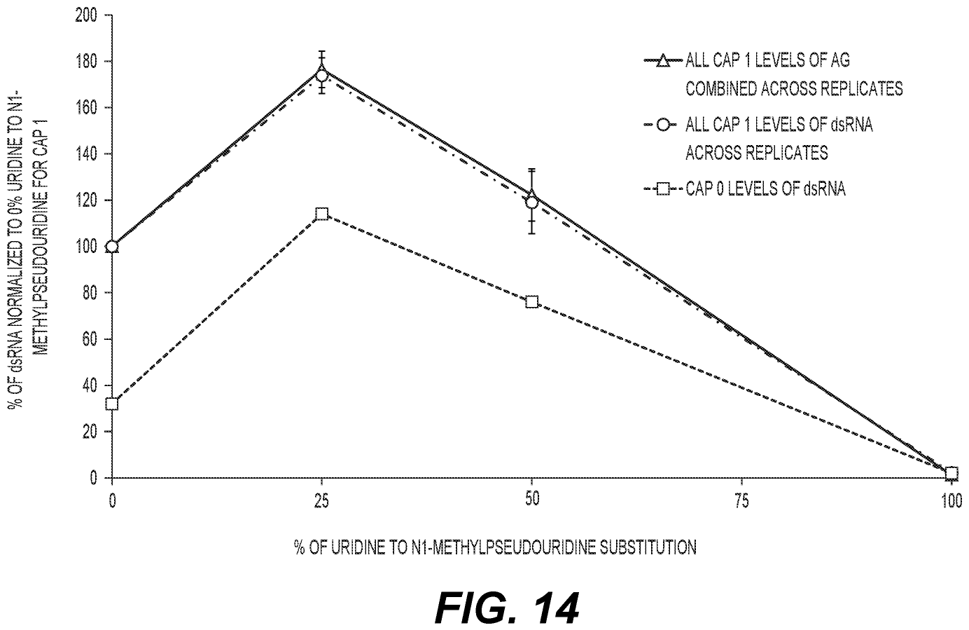

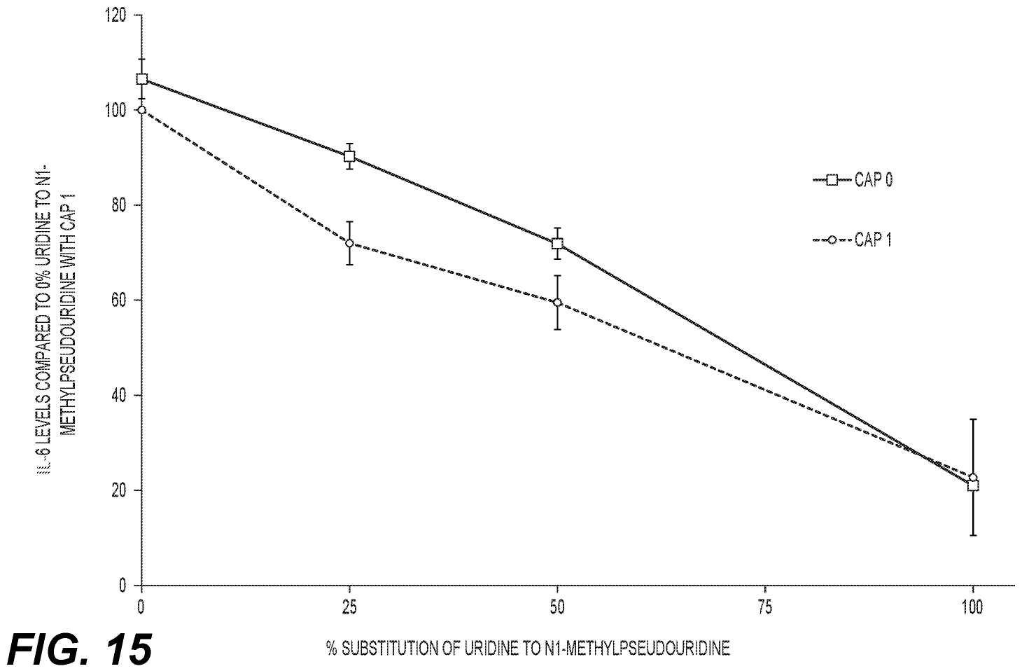

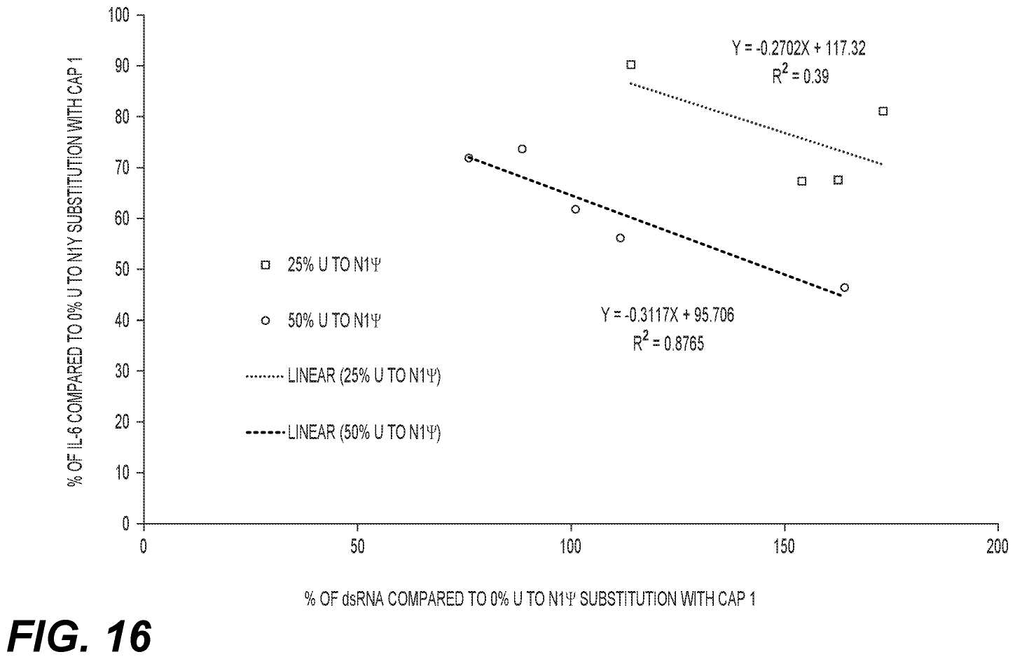

BJ cells were treated with SAM RNA as described above in Example 5. FIG. 13 depicts the correlation between antigen levels and double-stranded RNA levels in BJ cells transfected with 25%, 50%, and 100% U to N1Ψ RNA across varying lots of capped SAM RNA. The antigen levels and double-stranded RNA levels obtained with each of the substitutions were normalized to the same with 0% substitution, which represent levels of 100%. FIG. 14 depicts the levels in BJ cells of double-stranded RNA obtained from transfecting the cells with SAM RNA comprising 0%, 25%, 50%, or 100% U to N1Ψ substitution and capped with cap 1. The results are normalized to the levels obtained from the cells treated with the 0% substituted SAM RNA, which represent levels of 100%. The results for the 25% and 100% substitution were from four different lots of cap 1 capping of the SAM RNA, and the results for 0% and 50% substitution were from five different lots of SAM RNA. Twenty five percent substitution increases the amount of double-stranded RNA in BJ cells compared to the same measures with 0% and 50% substitution. Fifty percent substitution slightly increases the amount of double-stranded RNA in BJ cells compared to the same measures with 0%. One hundred percent substitution results in the lowest expression of double-stranded RNA when compared to that from all other percent substitutions.

FIG. 15 depicts the levels of interleukin 6 (IL-6) from the same BJ cells transfected with SAM RNA comprising cap 1 or cap 0 capping and 0, 25%, 50%, or 100% U to N1Ψ substitution as in FIG. 14. There is no interaction between substitution and capping on IL-6 levels from BJ cells. The levels of IL-6 drop significantly with each increment of substitution in both the cap 0 and cap 1 conditions. BJ cells release less IL-6 when treated with SAM with cap 1 than when treated with SAM with cap 0.

FIG. 16 depicts the negative correlations between IL-6 levels in FIG. 15 and the double-stranded RNA levels in FIG. 14 obtained from the BJ cells transfected with SAM RNA comprising 25% or 50% U to N1 W substitution and cap 1 capping within different lots of cap 1. Within 25% or 50% percent substitution, the amount of IL-6 negatively correlates with the amount of double-stranded RNA.

Based on these results, there are two independent thresholds sensitive to the percent substitution. The innate immune response inhibits the self-amplification of the SAM RNA. A first, lower threshold, in substitution of U to N1Ψ triggers evasion of the innate immune response thereby improving self-amplification. There is also a direct effect of substitution on inhibiting self-amplification. Fifty percent substitution results in lower double-stranded RNA levels than levels with 25% substitution, but these levels are higher than levels with 0% substitution. One hundred percent substitution results in almost no double-stranded RNA formation. There is therefore a second, higher, threshold between 50% and 100% substitution (i.e., ˜75% at its midpoint in the sigmoidal curve) whereby the substitution directly inhibits self-amplification.

In combination, there is a balance whereby some substitution increases self-amplification by evading, or lowering, the innate immune response, but too much substitution inhibits induction of an immune response to the antigen and thereby the pathogen by inhibiting self-amplification of the SAM. Even with some inhibition of self-amplification and antigen expression from a SAM with greater than 50% substitution, as administered, will suffice to provide an effective intracellular introduction of an heterologous nucleic acid, which may encode a heterologous protein such as an antigen to induce an immune response, or a protective immune response, to the antigen, and thereby the pathogen.

### Example 8

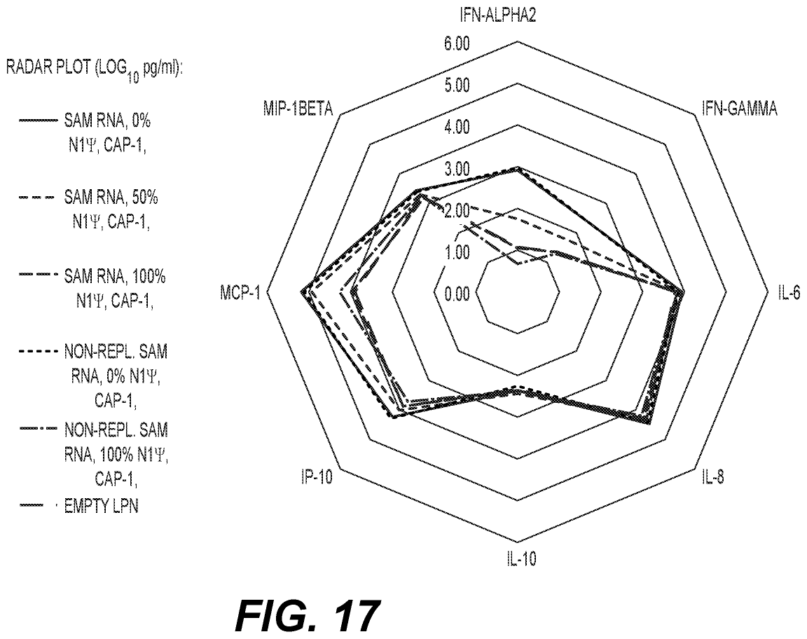

Human PBMC cells were treated as described in Example 6 and then were assessed for levels of 22 different cytokines, including those shown in FIG. 17.

FIG. 17 is the radar plot of levels of interferons-alpha and -gamma (IFN-α and IFN-γ); interleukins-6 (IL-6), -8 (IL-8), and -10 (IL-10); IFN-γ-induced protein-10 (IP-10, also known as C—X—C motif chemokine ligand-10 or CXCL-10); monocyte chemoattractant protein-1 (MCP-1); and macrophage inflammatory protein 1-beta (MIP-1β) obtained from a single donor's peripheral blood monocyte cells (PBMC) which were treated with non-replicating SAM RNA encoding firefly luciferase, comprising 0% or 100% substitutions of U to N1Ψ, and being capped with cap 1; or SAM RNA encoding firefly luciferase, comprising 0%, 50%, or 100% substitutions of U to N1Ψ, and being capped with cap 1.

Fifty percent substitution, compared to 0% substitution, reduced IFN-α and IFN-γ and trended toward reducing MCP-1, IL-10, IP-10, and MIP-1β levels but not IL-6 or IL-8 levels. One hundred percent substitution, compared to 0% substitution, reduced IFN-α, IFN-γ, IL-8, IP-10, MCP-1, and MIP-1β levels, but not IL-6 levels. The reductions in IP-10 confirm the reductions in IFN-γ, since the latter induces the former. Treatment with lipopolysaccharides (a toll-like receptor 4 agonist) and R848 (a.k.a. resiquimod, a TLR-7 and TLR-8 agonist) and the levels of IL-1α, IL-1β, IL-12, IL-18, IL-22, and tumor necrosis factor-α are not shown.

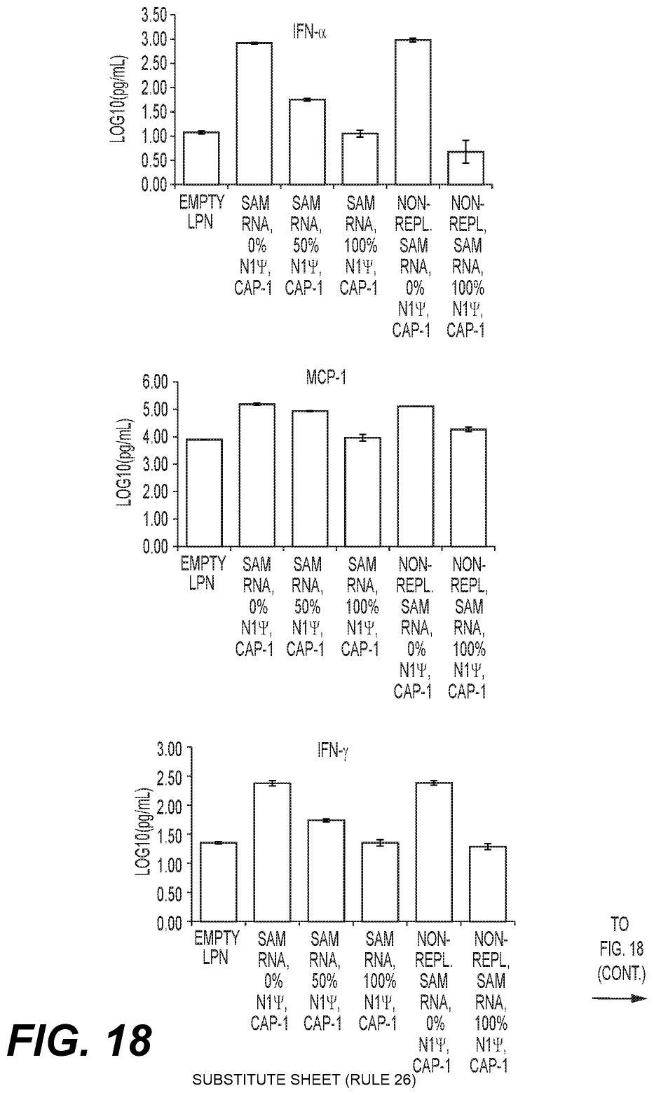

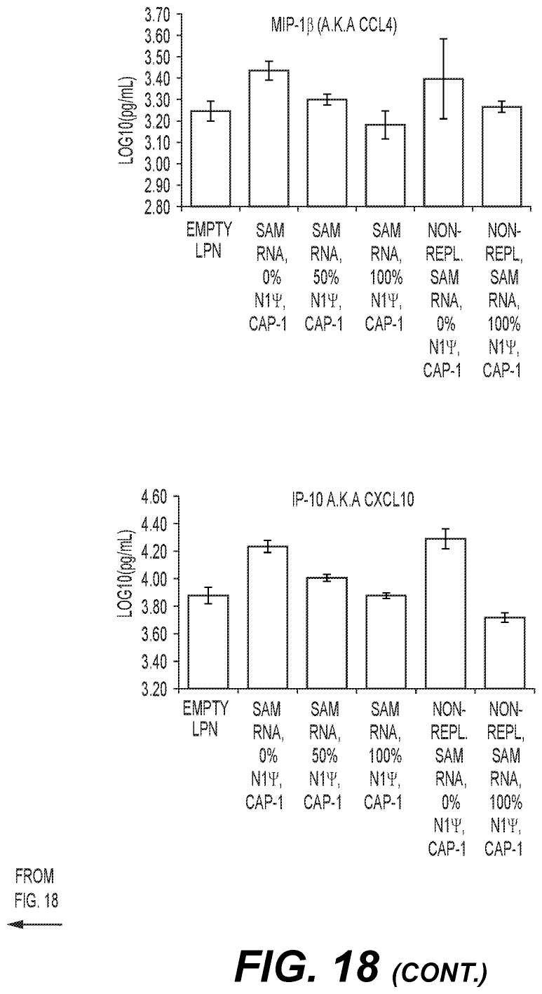

FIG. 18 depicts the histograms of levels of IFN-α, IFN-β, MCP-1, MIP-1β, and IP-10 from FIG. 17.

### Example 9

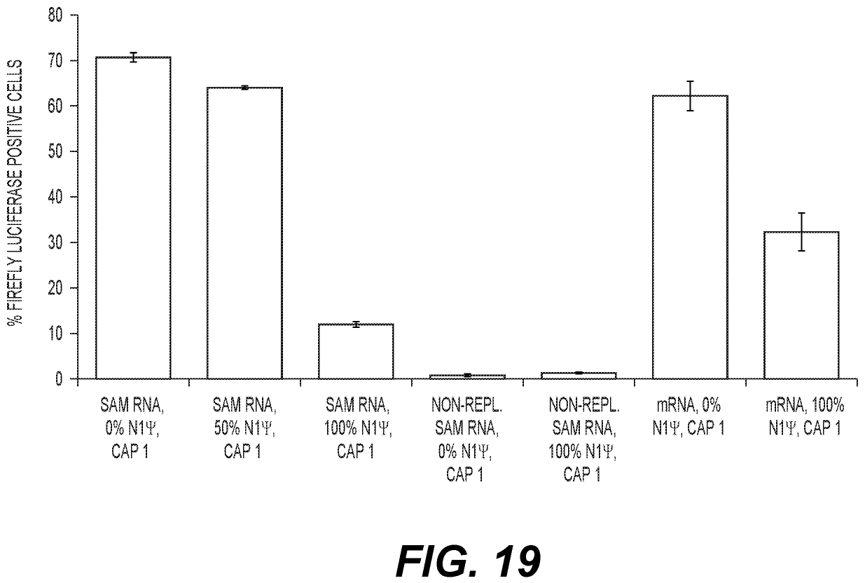

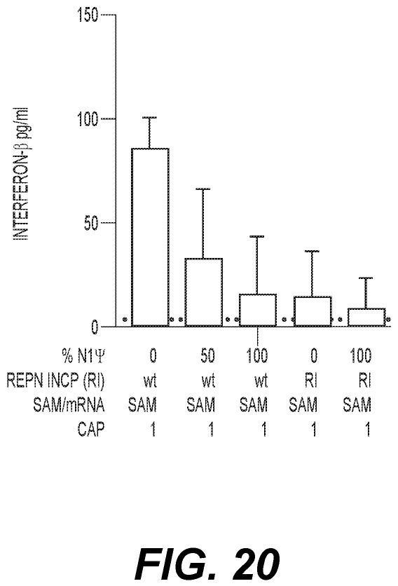

Human skeletal muscle cells were transfected with SAM RNA/LNP compositions as described above. Zero percent- and 50% substituted SAM transfect cells with similar efficiency while the 100% modified SAM is defective in self-amplification. FIG. 19 depicts these results as the percentage of positive human skeletal muscle cells (hSkM) upon transfection with mRNAs or SAM RNA encoding firefly luciferase and comprising 0%, 50%, or 100% substitutions of U to N1 W and being capped with cap 1.

FIG. 20 depicts the histograms of the levels of IFN-β from the cells of FIG. 19, showing reduction of IFN-β when a 50% substituted SAM is used. In comparison to the results of FIG. 19, the decrease in IFN-β with increasing substitution precedes the decrease in gene expression encoded within the SAM RNA with increasing substitution in hSkM, as with C2C12 cells.

### In Vivo Examples

### Example 10

This in vivo study assessed the innate activation and immunogenicity of GSK self-amplifying messenger RNA (SAM) produced in vitro with a cap1, in the presence of selected amounts of N1-methylpseudouridine (N1Ψ). The SAM RNA encodes the spike-protein (S-protein) from severe acute respiratory syndrome coronavirus 2 virus (SARS-CoV-2) within the TC83 backbone.

### Methods

### Mice

Six- to eight-week-old female BALB/c mice were injected with a 50 μL unit dose of RNA in both hindlegs (each hindleg was injected with 25 μL, which is half the unit dose) on day 0 (start of the study) and again day 21 according to the following groups and, within the group, number of animals, number of unit doses given in total, RNA modifications, LNP formulations, masses of RNA in the unit dose, and template plasmid DNA (SEQ ID NOS.: 5 or 6) used to in vitro transcribe the RNA (SEQ ID NO: 7 being the RNA obtained from in vitro transcribing SEQ ID NO: 6).

| | # | | | | Mass of | | |
|---|---|---|---|---|---|---|---|
| Group | Mice | # of | | | RNA in | | |
| Identifier | in the | Unit | | LNP | Unit | | SEQ ID |
| Number | Group | Doses | RNA | Formulation | Dose | | NO: |
| 1 | 13 | 2 | Saline | None | — | | |
| 2 | 13 | 2 | Empty LNP | RV39 | — | | |
| 3 | 13 | 2 | SAM cap-0 | RV39 | 3 | μg | 5 |
| 4 | 13 | 2 | SAM cap-1 | RV39 | 3 | μg | 5 |
| 5 | 13 | 2 | SAM cap-1, oligodT-cellulose | RV39 | 3 | μg | 5 |
| | | | purified | | | | |
| 6 | 13 | 2 | SAM cap-1, 50% N1Ψ, oligodT- | RV39 | 3 | μg | 5 |
| | | | cellulose purified | | | | |
| 7 | 13 | 2 | SAM cap-1, 25% N1Ψ, oligodT- | RV39 | 3 | μg | 5 |
| | | | cellulose purified | | | | |
| 8 | 13 | 2 | mRNA cap1, 100% N1Ψ, oligodT- | RV39 | 3 | μg | 5 |
| | | | cellulose purified | | | | |
| 9 | 10 | 2 | SAM cap-0 | RV39 | 0.15 | μg | 5 |
| 10 | 10 | 2 | SAM cap-1 | RV39 | 0.15 | μg | 5 |
| 11 | 10 | 2 | SAM cap-1, oligodT-cellulose | RV39 | 0.15 | μg | 5 |
| | | | purified | | | | |
| 12 | 10 | 2 | SAM cap-1, 50% N1Ψ, oligodT- | RV39 | 0.15 | μg | 5 |
| | | | cellulose purified | | | | |
| 13 | 10 | 2 | SAM cap-1, 25% N1Ψ, oligodT- | RV39 | 0.15 | μg | 5 |
| | | | cellulose purified | | | | |
| 14 | 10 | 2 | mRNA cap1, 100% N1Ψ, oligodT- | RV39 | 0.15 | μg | 6 |
| | | | cellulose purified | | | | |

The SARS-CoV-2 SAM RNA was delivered within RV39 lipid nanoparticles (LNP). The BALB/c mice were treated with one of the following: (1) saline (n=13), (2) empty lipid nanoparticle (i.e., no SAM RNA administered, n=13), (3) a 3 μg unit dose of cap-0 5′ capped SAM RNA comprising uridines but not N1-methyl pseudouridines (N1Ψ) and purified without oligodT-cellulose column purification in RV39 LNP (n=13), (4) a 3 μg unit dose of cap-1 5′ capped SAM RNA comprising uridines but not N1Ψ and purified without oligodT-cellulose column purification in RV39 LNP (n=13), (5) a 3 μg unit dose of cap-1 5′ capped SAM RNA comprising uridines but not N1Ψ and purified with oligodT-cellulose column purification in RV39 LNP (n=13), (6) a 3 μg unit dose of cap-1 5′ capped SAM RNA synthesized with an admixture comprising 50 mole % uridines and 50 mole % N1Ψ out of the total of uridines and N1Ψ in the admixture and purified with oligodT-cellulose column purification in RV39 LNP (n=13), (7) a 3 μg unit dose of cap-1 5′ capped SAM RNA synthesized with an admixture comprising 75 mole % uridines and 25 mole % N1Ψ out of the total of uridines and N1Ψ in the admixture and purified with oligodT-cellulose column purification in RV39 LNP (n=13), (8) a 3 μg unit dose of cap-1 5′ capped SAM RNA synthesized with an admixture comprising N1Ψ but no uridines and purified with oligodT-cellulose column purification in RV39 LNP (n=13), (9) a 0.15 μg unit dose of cap-OS5′ capped SAM RNA comprising uridines but not N1-methylpseudouridines (N1Ψ) and purified without oligodT-cellulose column purification in RV39 LNP (n=0), (10) a 0.15 μg unit dose of cap-1 5′ capped SAM RNA comprising uridines but not N1Ψ and purified without oligodT-cellulose column purification in RV39 LNP, (11) a 0.15 μg unit dose of cap-1 5′ capped SAM RNA comprising uridines but not N1Ψ and purified with oligodT-cellulose column purification in RV39 LNP (n=10), (12) a 0.15 μg unit dose of cap-1 5′ capped SAM RNA synthesized with an admixture comprising 50 mole % uridines and 50 mole % N1Ψ out of the total of uridines and N1Ψ in the admixture and purified with oligodT-cellulose column purification in RV39 LNP (n=10), (13) a 0.15 μg unit dose of cap-1 5′ capped SAM RNA synthesized with an admixture comprising 75 mole % uridines and 25 mole % N1Ψ out of the total of uridines and Nip in the admixture and purified with oligodT-cellulose column purification in RV39 LNP (n=0), or (14) a 0.15 μg unit dose of cap-1 5′ capped SAM RNA synthesized with an admixture comprising N1Ψ but no uridines and purified with oligodT-cellulose column purification in RV39 LNP (n=0).

The above-noted formulations had the stock concentrations, lot numbers, vials, and storage temperatures indicated in Table 3.

TABLE 3

| | | | | Storage |
|---|---|---|---|---|
| | | Stock | Number | Temper- |
| | Lot | Concen- | of vials | ature |
| Test Article Label | Number | tration | supplied | (°C) |
| SAM cap-0, RV39 | N78957- | 60 | 3 vials (1 mL | −80° C. |
| LNPs | 24a | μg/mL | material per | |
| | | | vial) | |
| SAM cap-1, RV39 | N78957- | 60 | 3 vials (1 mL | −80° C. |
| LNPs | 24b | μg/mL | material per | |
| | | | vial) | |
| SAM cap-1, which was | N78957- | 52 | 3 vials (1 mL | −80° C. |
| oligodT-cellulose | 24c | μg/mL | material per | |
| purified, RV39 LNPs | | | vial) | |
| SAM cap-1, 50% N1Ψ, | N78957- | 60 | 3 vials (1 mL | −80° C. |
| which was oligodT- | 24d | μg/mL | material per | |
| cellulose purified, | | | vial) | |
| RV39 LNPs | | | | |
| SAM cap-1, 25% N1Ψ, | N78957- | 60 | 3 vials (1 mL | −80° C. |
| which was oligodT- | 24e | μg/mL | material per | |
| cellulose purified, | | | vial) | |
| RV39 LNPs | | | | |
| mRNA cap1, 100% | N78957- | 60 | 3 vials (1 mL | −80° C. |
| N1Ψ, which was | 24f | μg/mL | material per | |
| oligodT-cellulose | | | vial) | |
| purified, RV39 LNPs | | | | |
| (RV39) | | | | |
| Empty LNPs | N78957- | — | 3 vials (1 mL | −80° C. |
| (RV39) | 24g | | material per | |
| | | | vial) | |

For cytokine analysis, serum was collected from groups 1-8 six hours (h) (8 mice/group) and twenty-four hours (5 mice/group) after the day 0 and day 21 injections. For groups 9-14, serum was collected on day 21 to measure antibody binding but before administration of the second unit dose. To measure total antibody levels against SARS-CoV-2 spike protein and neutralizing antibody levels against SARS-CoV-2 spike protein, blood was collected from all groups on day 35. Splenocytes (5 mice/group) were harvested on day 35 to measure the T follicular helper (Tfh) cell responses and antigen-specific T cell and B cell responses.

### Cytokine Assay

Cytokines were measured with the U-PLEX™ Biomarker Multiplex Assay (MESOSCALEDISCOVERY™ (MSD), cat. no K15069L) according to the manufacturer's instructions. Briefly, individual U-PLEX™ linker-coupled antibody solutions were prepared by adding 200 μL of biotinylated antibody to 300 μL of the assigned linker and incubated for 30 minutes. After 30 minutes, 200 μL of stop solution was added, then the mixture was incubated for an additional 30 minutes. Then, the multiplex coating solution was prepared by combining 600 μL of each U-PLEX™ coupled antibody solution. Fifty microliters of the multiplex coating solution was added to each well on the U-PLEX™ plate. The plate was sealed and incubated for 1 hour at room temperature with shaking. After incubation, the plate was washed 3 times with 1×MSD Wash Buffer. Calibrator standard solutions were prepared according to MSD specifications. Serum samples were diluted 1:2 with Diluent 41. Next, 25 μL of Diluent 41 was added to each well. Then, 25 μL of the standards and diluted serum were added to the wells and the plate was sealed and incubated for 1 hour at room temperature with shaking. After incubation, the plate was washed 3 times with 1×MSD Wash Buffer. Detection antibody solution was prepared immediately before use according to the manufacturer's instructions and 50 μL was added to each well. The plate was sealed, incubated at room temperature with shaking for 1 hour, and then washed 3 times with 1×MSD Wash Buffer. 150 μL of MSD Gold Read Buffer B was added to each well and the plate was analyzed on an MSD instrument (Meso Sector S 600 with Discovery Workbench Software v4.0).

### Total SARS-CoV-2 Spike Protein-Specific Antibody Titers

A multiplex assay was performed to evaluate SARS-Cov2 spike-specific antibodies in the serum of the immunized mice. LUMINEX™ microspheres (MAGPLEX™ microspheres, LUMINEX™ Corp from Austin, TX) were coupled with SARS-Cov2 spike antigen (lot: N79663) at 10 μg/mL reaction by chemical coupling according to manufacturer instructions. In 96 well plates, about 1000 microspheres/well were added in a volume of 50 μL phosphate-buffered saline (PBS) with 1% bovine serum albumin (BSA)+0.05% sodium azide (assay buffer) into five-fold serial dilutions of mouse serum. The microspheres and serum were incubated on an orbital shaker (covered) for 60 minutes at room temperature. The microspheres were washed one time three times with 200 μL per well of PBS with 0.05% Tween-20 (wash buffer) on a plate washer using a magnet to allow settling of beads between washes. Following the wash, 50 μL per well of r-Phycoerythrin (r-PE) conjugated anti-mouse immunoglobulin G (IgG) (Jackson Immunoresearch) was added at a 1:50 dilution, and plates were incubated (covered) on an orbital shaker at room temperature for 60 minutes. The plates were washed with the methods described above and incubated with PBS (covered) at room temperature on an orbital shaker for 20 minutes. Fluorescent intensity was measured using a Luminex INTELLIFLEX™ FLEXMAP 3D™ (Life Technologies model INTELLIFLEX FM3D000). The raw data was analyzed using a Softmax Pro template, where the serum sample binding potency was interpolated based on a five-parameter logistic fit of the standard curve. Serum anti-SARS-CoV2 spike potency was calculated in terms of Assay Units (AU) using a reference standard. In brief, a cocktail of monoclonal antibodies (mAb) specific for SARS-CoV-2 spike were prepared (1A9 Genetex #75870-168, mCR 3022 Absolute Antibody #AB1680-3.0, and MP 0142 MP Biologicals #8720412) at an optimized dilution (1:17.36 for 1A9, 1:11.6 for mCR 3022, and 1:9.63 for MP 0142). The readout from this mAb cocktail was assigned a concentration of 100 AU for standard normalization purposes.

### Anti-SARS-CoV-2 Neutralization Antibody Titers

Serum samples were analyzed using a validated pseudovirion neutralization assay (PNA) against the Wuhan (WT) variant at Nexelis. On day 1 of the neutralization assay (which is discrete from the days of in vivo treatments), Vero E6 cells expressing the ACE-2 receptor were seeded in 96-well white plates at 20,000 cells/well to reach a cell confluence of 80% next day. On day 2, serum samples and controls were serially diluted. In parallel, SARS-CoV-2 pseudovirus was diluted to reach the desired concentration (according to pre-determined TU/mL). Pseudovirus was added to diluted serum samples and pre-incubated for 1 hour at 37° C. with 5% CO2. The mixture was added to the pre-seeded Vero E6 cell layers and plates were incubated for 18-22 hours at 37° C. with 5% CO2. On day 3, following incubation and removal of media, One-Glo EX luciferase assay substrate was added to cells and incubated for 3 minutes at RT with shaking. Luminescence of the luciferase was measured using a SPECTRAMAX™ iD3 microplate reader and SoftMax Pro v7.0.1. Luminescence results for each dilution were used to generate a titration curve using a 4-parameter logistic regression (4PL). The titers were defined as the reciprocal dilution of the sample for which the luminescence is equal to a pre-determined cut-point of 50, corresponding to 50% neutralization. This cut-point was established using linear regression using 50% flanking points. Samples were analyzed using an array of acceptance criteria and were repeated if an acceptance criterion is not met.

### Intracellular Cytokine Staining by Flow Cytometry

Multi-parameter flow-cytometry with intracellular cytokine staining (ICS) was used to assess SARS-CoV-2 Spike-specific CD4 and CD8 T cell responses. Spleens were harvested on day 35 from 5 mice per group. Splenocytes from individual mice were plated in round-bottom 96 well tissue culture plates at 1.5×10⁶ viable cells/well and stimulated with one of the following pools of spike peptides (from JPT Peptide Technologies Gmbh, Berlin, Germany or Genscript):

| | | Stimu- | | |
|---|---|---|---|---|
| | No. | lation | Vendor | |
| | Peptides | Concen- | (Catalog | |
| Stimulation | in Pool | tration | Number) | Lot No. |
| Media | N/A | N/A | N/A | N/A |
| Spike Peptide | 158 | 1.0 | JPT Peptide | 47886VSi- |
| Pool 1 70% | | μg/mL | Technologies | 01 |
| Purity | | | Gmbh (PM- | |
| | | | WCPV-S-2, | |
| | | | vial 1) | |
| Spike Peptide | 157 | 1.0 | JPT Peptide | 47886VSi- |
| Pool 2 70% | | μg/mL | Technologies | 02 |
| Purity | | | Gmbh (PM- | |
| | | | WCPV- | |
| | | | S-2, vial 2) | |
| Spike full | 6 | 1.0 | Genscript | U260NFE270 |
| length gap | | μg/mL | | |
| peptides | | | | |
| Spike Ecto | 8 | 1.0 | Genscript | U260NFE270 |
| gap peptides | | μg/mL | | |

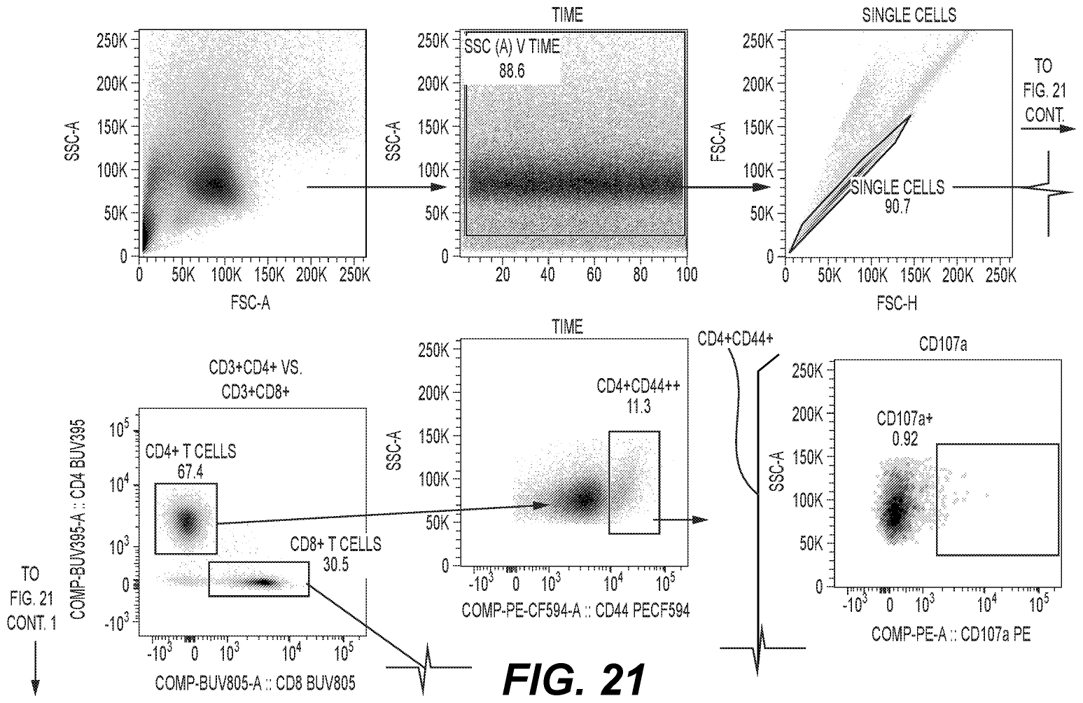

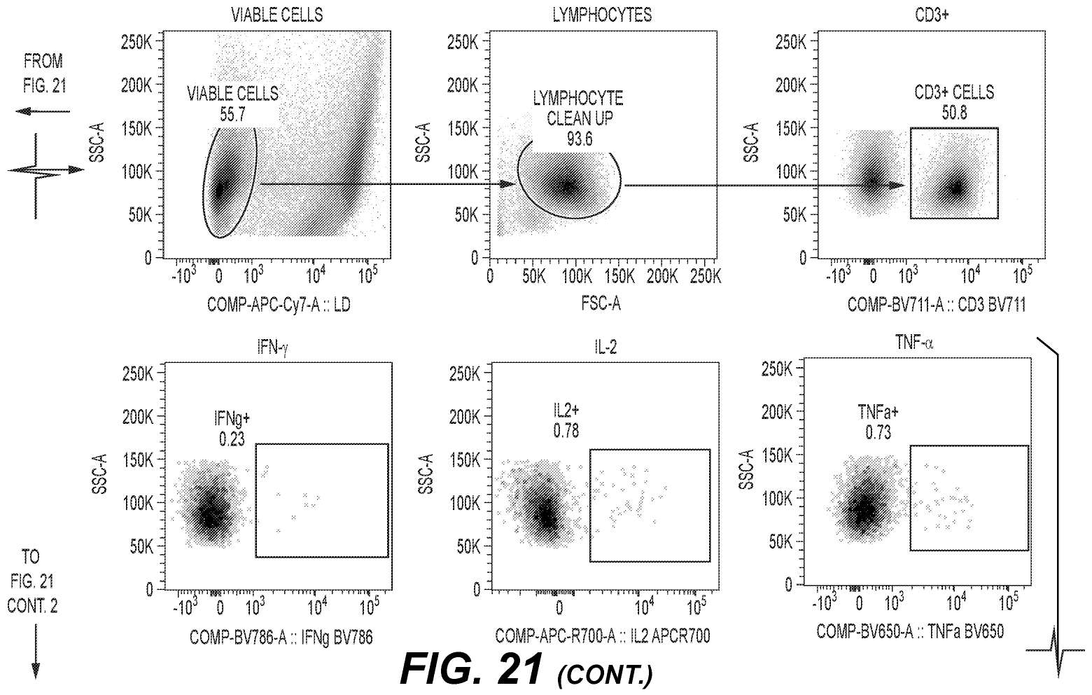

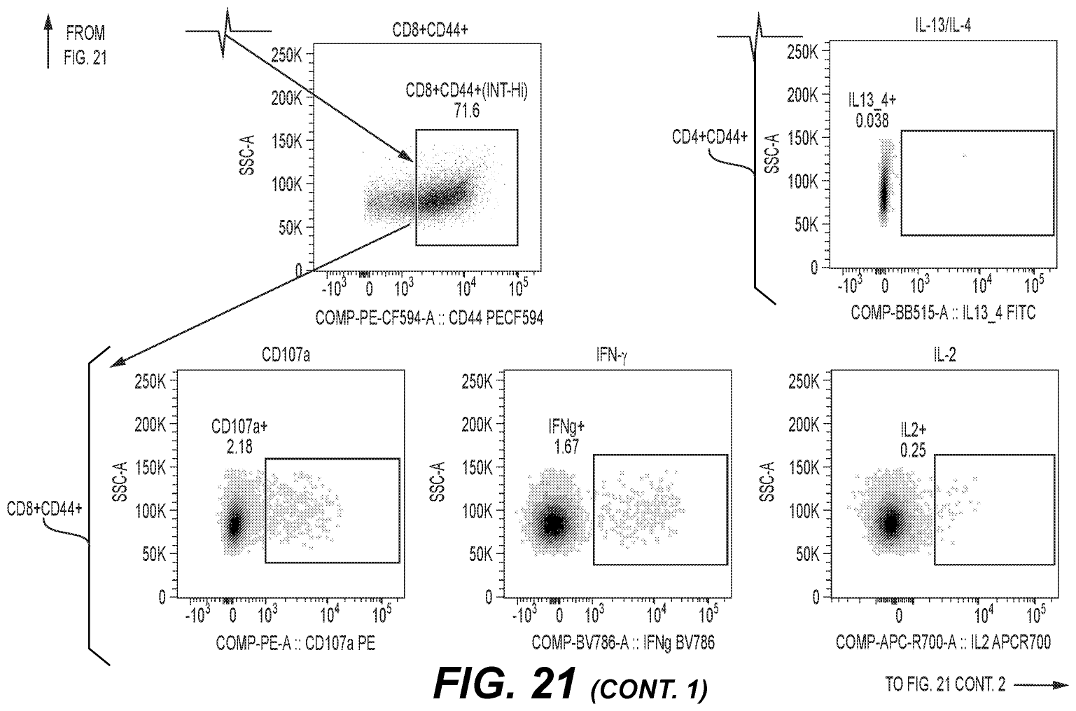

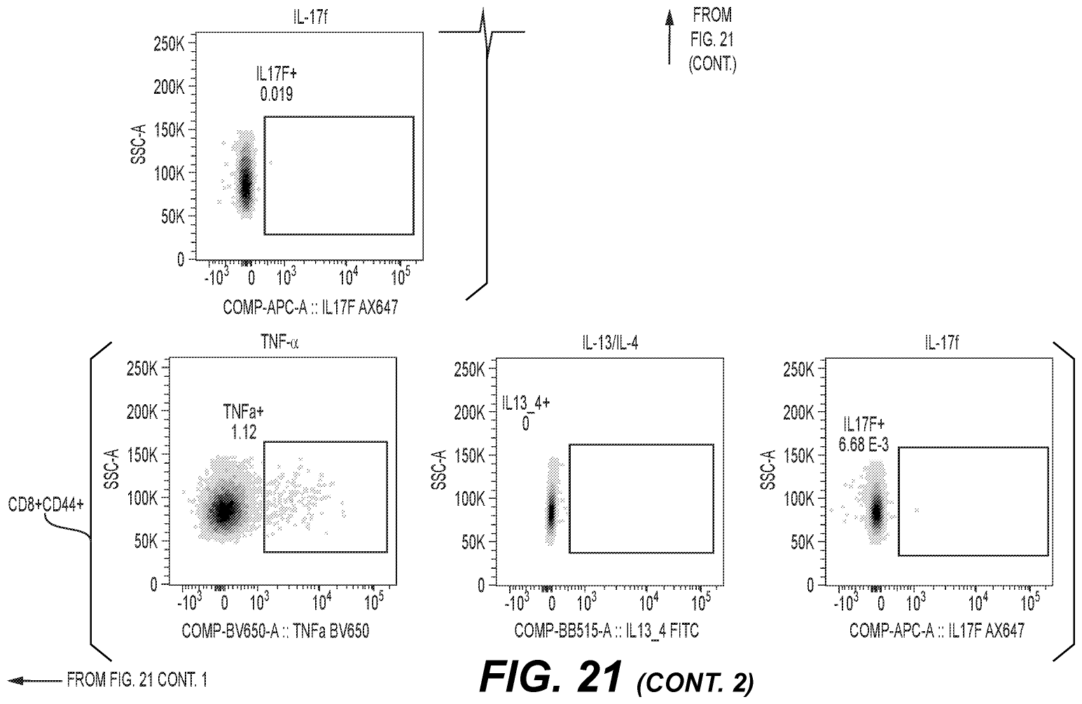

All samples were co-stimulated with the anti-CD28 clone 37.51 monoclonal antibody (mAb, ThermoFisher, Catalog No. 14-0281-82) and stained for the degranulation marker CD107a, 12-14 hours, in culture (37° C., 5% CO2) for 12-14 hours, thereby performing a stimulatory incubation. Two hours into the stimulatory incubation, BD GOLGIPLUG™, a protein transport inhibitor containing Brefeldin A, was added to all samples to block cytokine secretion from the cell. Upon completion of the stimulatory incubation, all samples were stained for: 1) viability, 2) phenotypic markers a) CD3, b) CD4, c) CD8, and d) CD44, and 3) cytokines a) IFN-γ, b) IL-2, c) TNF-α, d) IL-17F, and e) IL-4/IL-13. The data was acquired on the BD FAC SYMPHONY™ A5 special order research product cell analyzer using the High Throughput Sampler (HTS) option for acquisition in a 96 well plate format. Prior to analysis, the raw .fcs data files were analyzed by the computational algorithm Flow AI, which is a quality control process that identifies and removes aberrant events caused by anomalies in fluorescence signal across all acquired parameters or flow rate over time of data acquisition. The flow cytometry data analysis was performed using FLOWJO™ v10.8.0. FIG. 21 illustrates the gating strategy used to identify activated CD4 and CD8 T cells. First, gates were defined that identified cytokine and CD107a expressing positive populations within the viable, activated, CD4 and CD8 T cell populations. Next, the Boolean combination gate tool was used to automatically generate all possible combinations (positive and negative) of cytokine and CD107a expressing cells. For activated CD4 and CD8 T cells, all six analytes were included in the Boolean analysis generating a total of 64 multi-functional subsets. The raw data was exported into Microsoft Excel for further analysis. The response to peptide pool stimulation was determined by subtracting the response in the unstimulated media control for each sample. Negative values resulting from this subtraction were identified and valued as null (“zero”). The multi-functional subsets were used to categorize the data into CD4 T helper (Th) and CD8 T cytotoxic (Tc) phenotypic subsets based on production of IFN-γ, IL-13/IL-4, and IL-17F cytokines, and these subsets are defined in Table 4.

TABLE 4

| Phenotype | IFN-γ | IL-13/IL-4 | IL-17F | IL-2 | TNF-α | CD107a |
|---|---|---|---|---|---|---|
| Tₕ/T_{c}0 | − | − | − | +/− | +/− | +/− |
| Tₕ/T_{c}1 | + | − | − | +/− | +/− | +/− |
| Tₕ/T_{c}2 | − | + | − | +/− | +/− | +/− |
| Tₕ/T_{c}17 | − | − | + | +/− | +/− | +/− |

All graphs of the intracellular cytokine staining by flow cytometry were generated with GraphPad Prism v8.0.0.

### Tfh Staining by Flow Cytometry

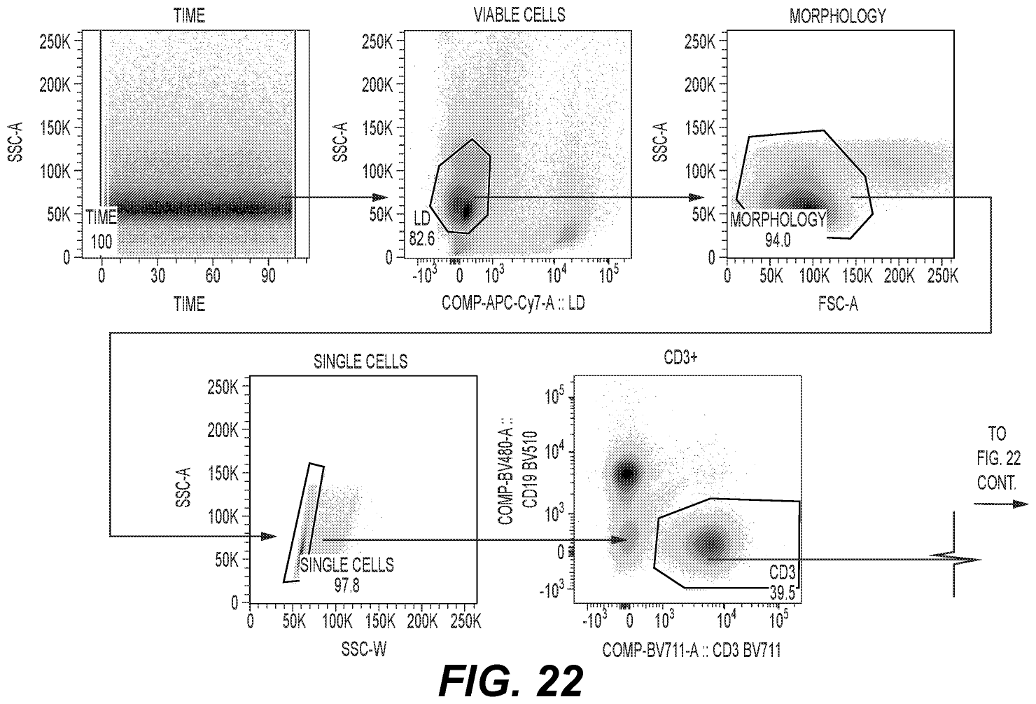

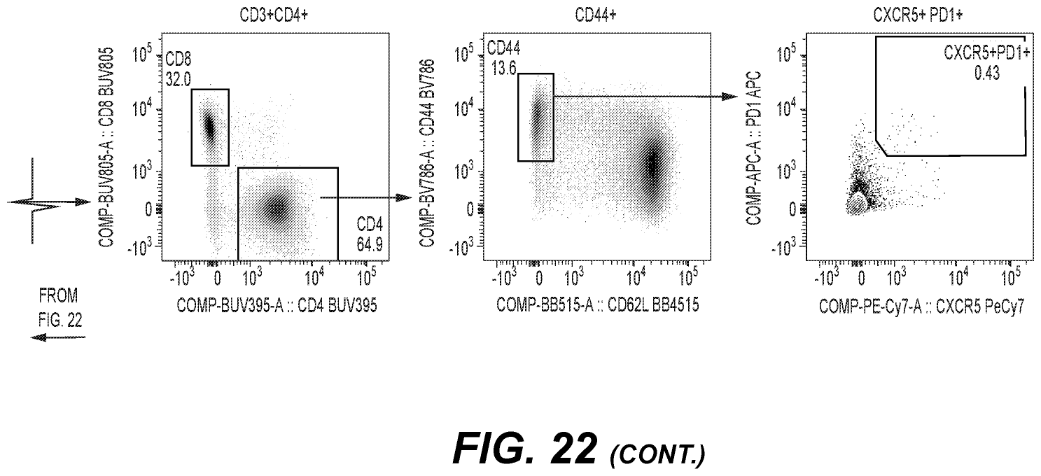

Spleens were collected from 5 mice per group at day 35. Splenocytes from individual mice were plated in round-bottom 96 well tissue culture plates at 1.5×10⁶ viable cells per well. Cells were stained with 1:1000 near IR Live/Dead stain (Invitrogen) for 20 min at room temperature and then treated with 1:15 diluted Fc block (BD Biosciences) in fluorescence-activated single cell sorting (FACS) wash buffer (PBS plus 1 volume % Fetal Bovine Serum, Thermo Scientific) for 10 min at 4° C. to avoid non-specific binding. Cells were then stained with the following extracellular markers: CD19-BV510, CD62L-BB515, CD127-BUV737, CXCR5-PECy7, PD-1-APC, CXCR3-BB700, CCR6-BV605, and ICOS-BV421, in Brilliant Stain Buffer (BSB) and FACS Wash Buffer. Cells were incubated for 20 minutes in the dark and fixed and permeabilized using BD cytofix/cytoperm solution for 20 minutes at 4° C. in the dark. After incubation, the cells were treated with 1:15 dilution of Fc Block for 10 min, and then stained with an intracellular antibody mix: CD3-BV711, CD4-BUV395, CD8-BUV805, CD44-BV786, and Bcl6-PE-CF594 in BSB in FACS Perm/Wash Buffer (10×BD Perm/Wash diluted to 1× in Ultra Pure Distilled Water) for 30 minutes at room temperature (in the dark). The cells were subsequently washed then resuspended in FACS Wash Buffer and acquired on the BD FACSYMPHONY™ A5 special order research product cell analyzer. Data was analyzed using FlowJo v10.8.0. Tfh cells were identified according to the gating strategy in FIG. 22.

B cell Staining by Flow Cytometry

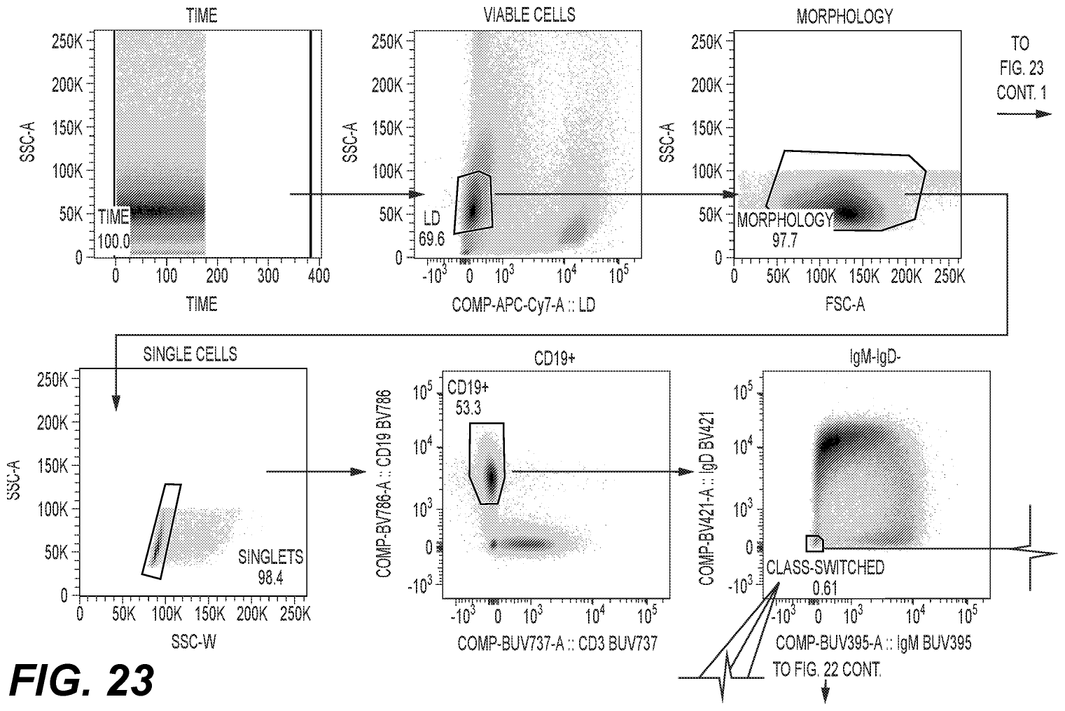

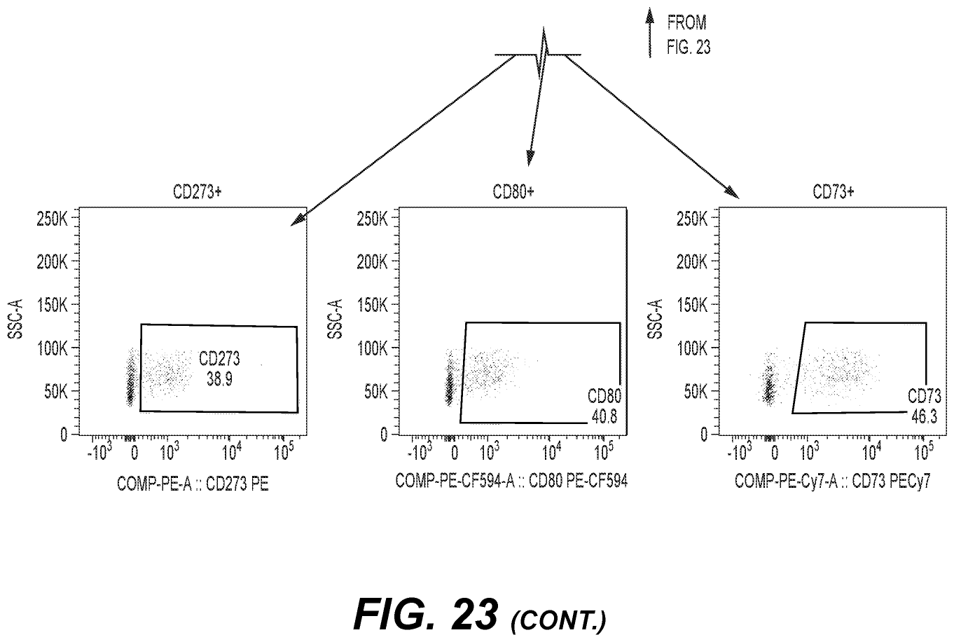

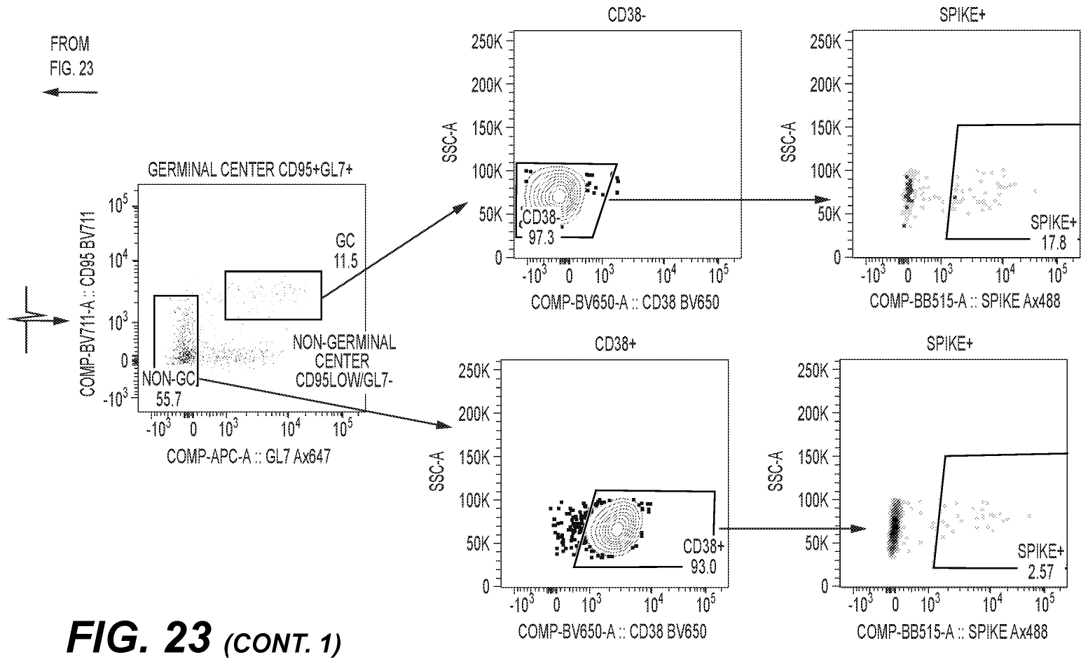

Multi-parameter flow cytometry was used to characterize SARS-CoV-2 spike-specific B cells in the spleen at day 35. Splenocytes from individual mice were plated in V-bottom 96 well tissue culture plates at 3×10⁶ viable cells per well. Cells were stained with near IR live/dead cell stain (Invitrogen) for 20 min at room temperature and then incubated with Fc block (BD Biosciences) in FACS Wash Buffer (PBS plus 1 volume % FBS, Thermo Scientific) for 10 min at room temperature. Following the incubation, the cells were extracellularly stained with the following antibodies: CD3-BUV737, CD19-BV786, IgD-BV421, IgM-BUV395, GL7-Ax647, CD95-BV711, CD38-BV650, CD138-BB700, CD73-PE-Cy7, CD273-PE, CD80-PE-CF594, and SARS-CoV-2 spike protein (GSK internal) fluorescently-labeled with an Ax488 labeling kit (Thermofisher). The fluorescence of the cells were acquired on BD FACSYMPHONY™ A5 special order research product cell analyzer and data analyzed with FLOWJO™ software v10.8.0 (BD Biosciences). Data was analyzed using FLOWJO™ v10.8.0 and spike-specific B cells were identified according to the gating strategy in FIG. 23. To identify spike-specific B cell phenotype, the gating for CD273, CD80, and CD73 were established based on IgM−IgD− cells (indicated in the gating scheme in FIG. 23). These gates were applied to the spike-specific B cell population, and Boolean combination gate tool was used to automatically generate all possible combinations (positive and negative) of spike-specific CD273, CD80, and CD73, the B cell memory populations of which are provided in Table 5.

TABLE 5

| Phenotype | CD273 (PD-L2) | CD80 | CD73 |
|---|---|---|---|
| Triple Negative | − | − | − |
| Single Positive | − | − | + |
| Transitional | + | − | +/− |
| L2/80 hi | + | + | − |
| Triple positive | + | + | + |

(+) positive;
(−) negative;
(+/−) positive or negative

### Serum Cytokines

A linear mixed model was fitted on the log 10-response including factor “group” (Group 1-8), the injection regimen (“Inj”, Vacc1 and Vacc2), the sample collection time point (“TP”, 6 h and 24 h) and their interactions as fixed effect and considering the repeated measurements (at PI and PII vaccination). The variance-covariance (VC) matrix was modelled considering the repeated measurements structures of type compound symmetry (CS), which assumes the same variance at PI and PII, or unstructured (Un), which consider different variances at PI and PII. The heterogeneity of variance was considered when appropriate by including the factor of heterogeneity in the VC design according to the grouping in Table 6.

TABLE 6

| Cytokine | VC structure | Grouping |
|---|---|---|
| IFN-β | CS | Group |
| IFN-γ | N/A | N/A |
| IL-6 | CS | N/A |
| IP-10 | CS | TP |
| KC/GRO | CS | TP |
| MCP-1 | Un | TP |
| MIP-1B | CS | TP |
| MIP-2 | Un | TP |
| TNF-α | CS | TP |

### Statistics

Interferon-gamma was fitted without considering the repeated measures as it was the most parsimonious model. The geometric mean of concentrations (GMC) and the geometric mean of ratios (GMR) and their 95% confidence interval (CI) were derived from the model.

### Total Anti-Spike Protein for SARS-CoV-2 Antibody Titers

A one-way ANOVA was performed on the log₁₀ of the anti-SARS-CoV-2 specific antibody titers on day 35. Groups 1 and 2 were excluded from the model as no response was detected. The geometric mean of concentrations (GMC) and the geometric mean of ratios (GMR) and their 95% CI were determined.

### Anti-SARS-CoV-2 Neutralization Antibody Titers

The distribution of the data was assumed to be lognormal.

A one-way ANOVA was performed on the log₁₀ of the anti-SARS-CoV-2 neutralization antibody titers. The variance was considered homogenous across groups. Group 1, 2 and 14 were excluded from the model as no response was detected. The geometric mean of concentrations (GMC) and the geometric mean of ratios (GMR) and their 95% CI were derived from the model.

### Flow Cytometry

The flow cytometry data, provided as proportions, were assumed to follow a zero-inflated β-distribution. The β-distribution function was re-parameterised as to generate parameters for the centrality and the scale. Considering that adaptation, separate regression models were fitted on the centrality and the scale parameters including factor “group” as fixed effect. The scale was either considered homogenous across groups or modelled on the groups. The probability of observing values equal to zero was conjointly modelled by a logistic regression model. The Bayesian SAS MCMC procedure was used to generate the parameters posterior distribution of the 3 regressions (i.e., centrality, scale and probability to observe zeros). The selected priors on the parameters were non informative, i.e., normal distributions with mean zero and variance 1E+06. The posterior distributions of the regression parameters were used to compute the posterior weighed means of the groups, which were then used to estimate the median and the 95% interquartile range (IQR). The posterior weighed means distributions were thereafter used to generate the posterior group ratios distribution, followed by the estimation of ratios median and 95% IQR.

### Results

### Total SARS-CoV-2 Spike Protein-Specific Antibody Titers

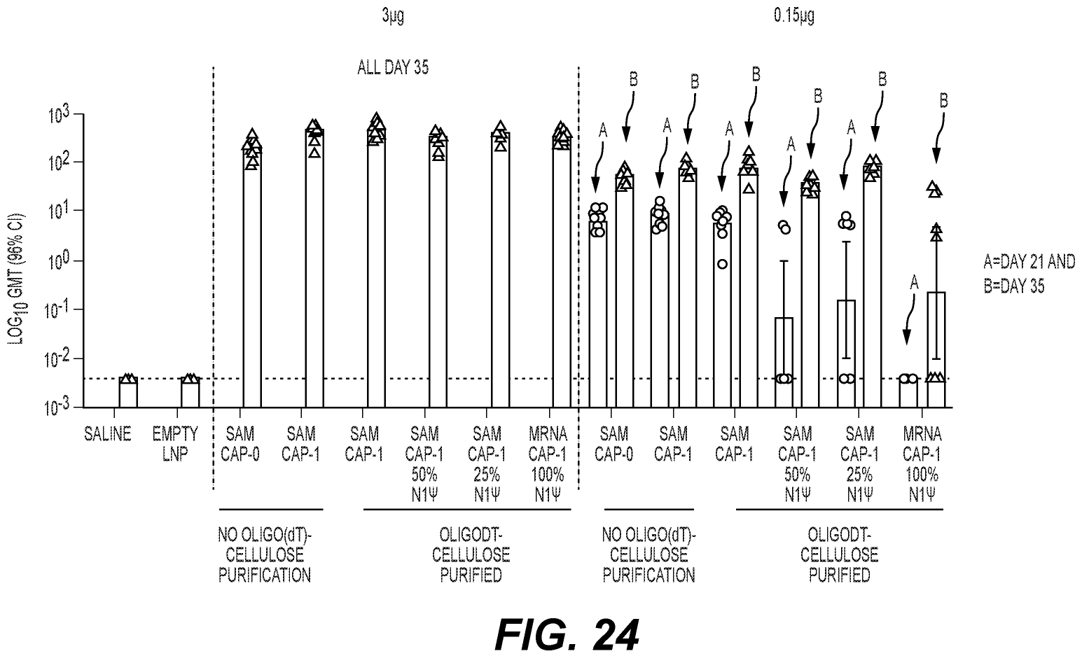

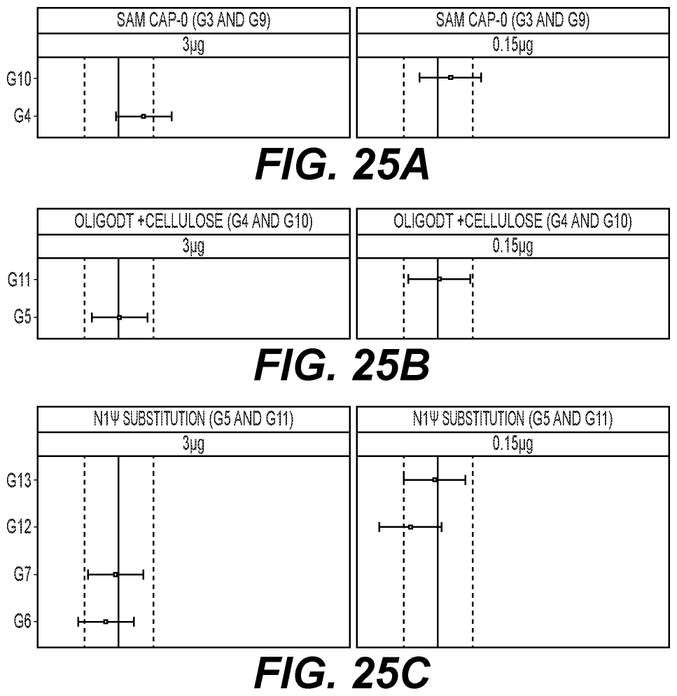

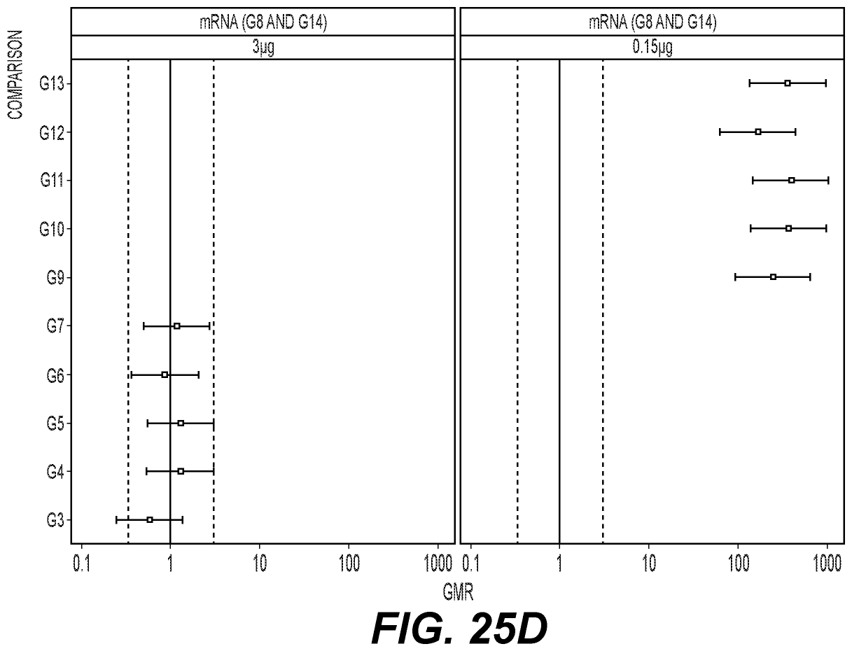

As noted above, total SARS-CoV-2 spike protein-specific antibody titers were measured in serum at day 21 for groups that received a 0.15 μg unit dose and at day 35 for all of the groups. Administering cap-1 5′ capped RNA did not significantly increase antibody levels when compared to antibody levels administration of cap-0 5′ capped RNA. See FIGS. 24, 25A, 25B, 25C, and 25D. Administering RNA purified with oligo(dT)-cellulose did not significantly increase the total antibody titers in comparison to the administration of RNA which was not oligo(dT)-cellulose purified. Administering SAM RNA at, for example, a lower unit dose of 0.15 μg, can significantly increase total SARS-CoV-2 spike protein-specific antibody titers when compared to those from animals administered mRNA (non-replicating), which comprised N1-methylpseudouridine but not uridine.

### Anti-SARS-CoV-2 Neutralization Antibody Titers

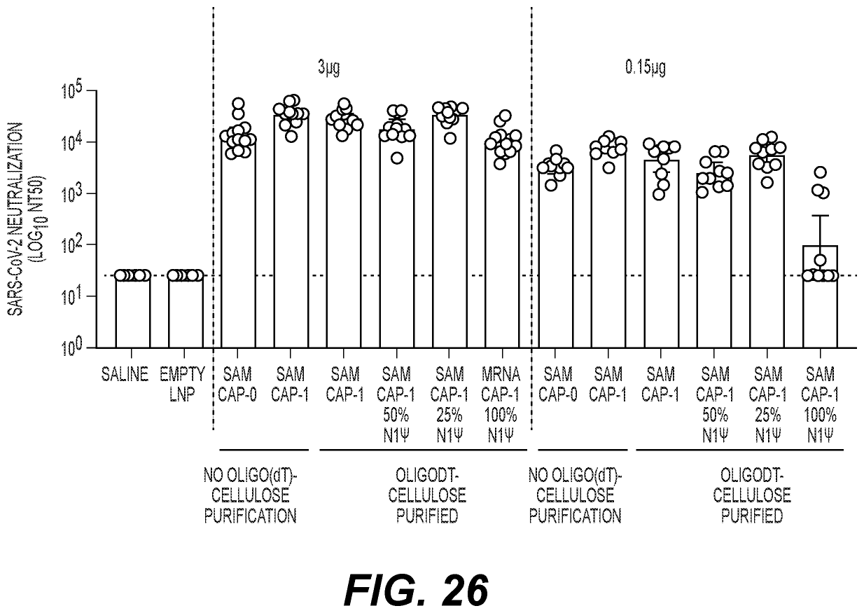

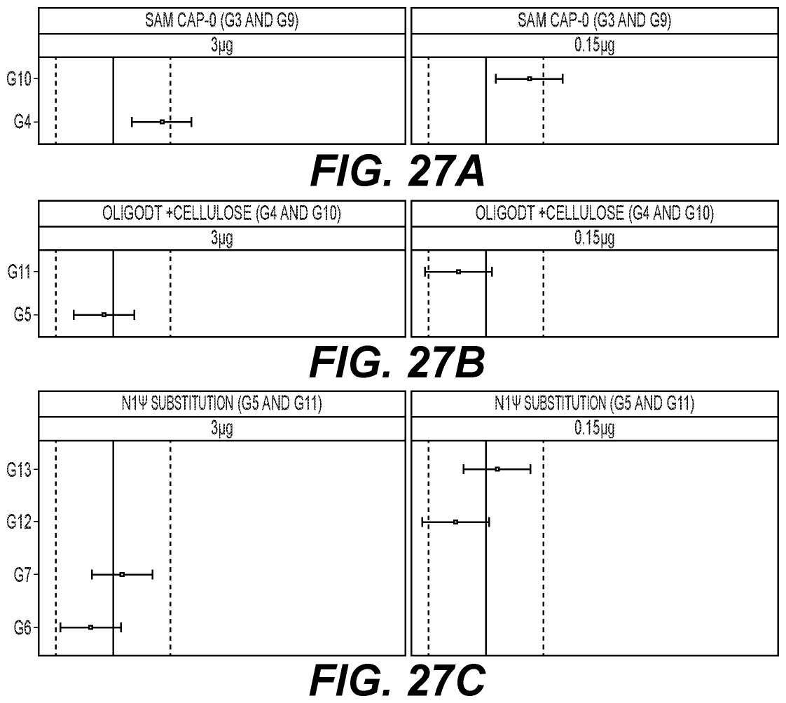

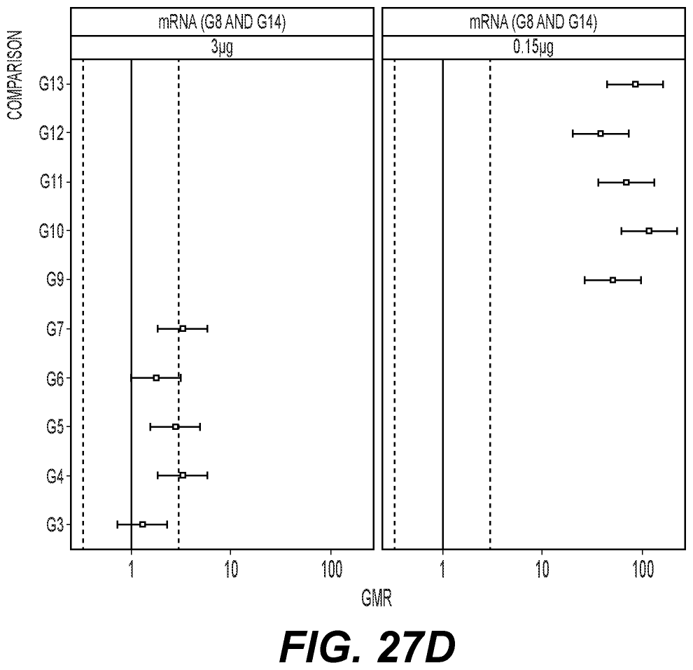

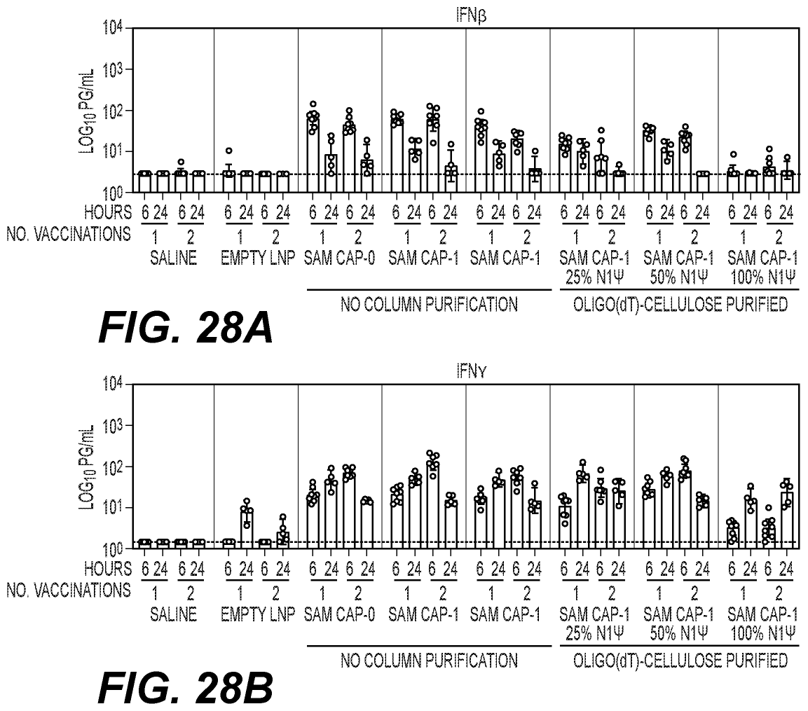

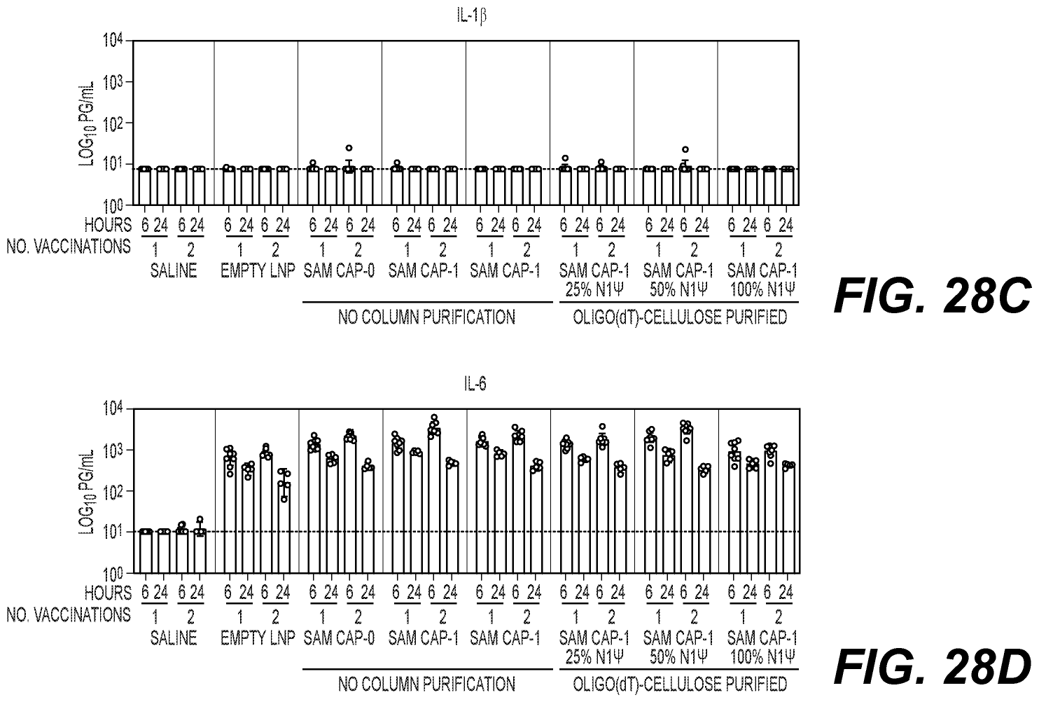

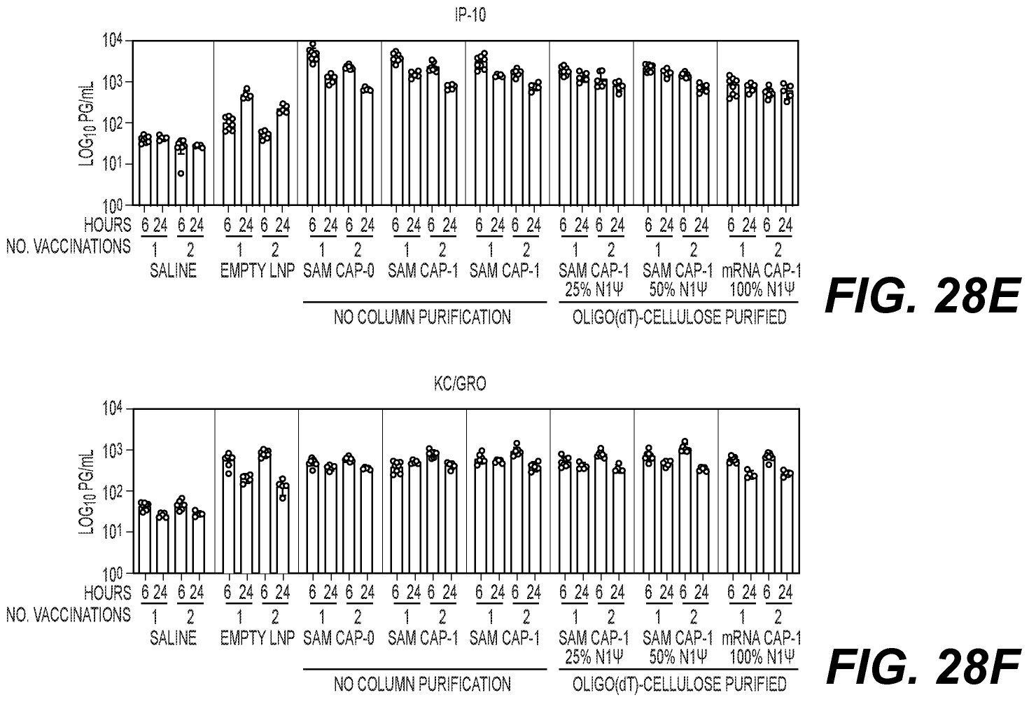

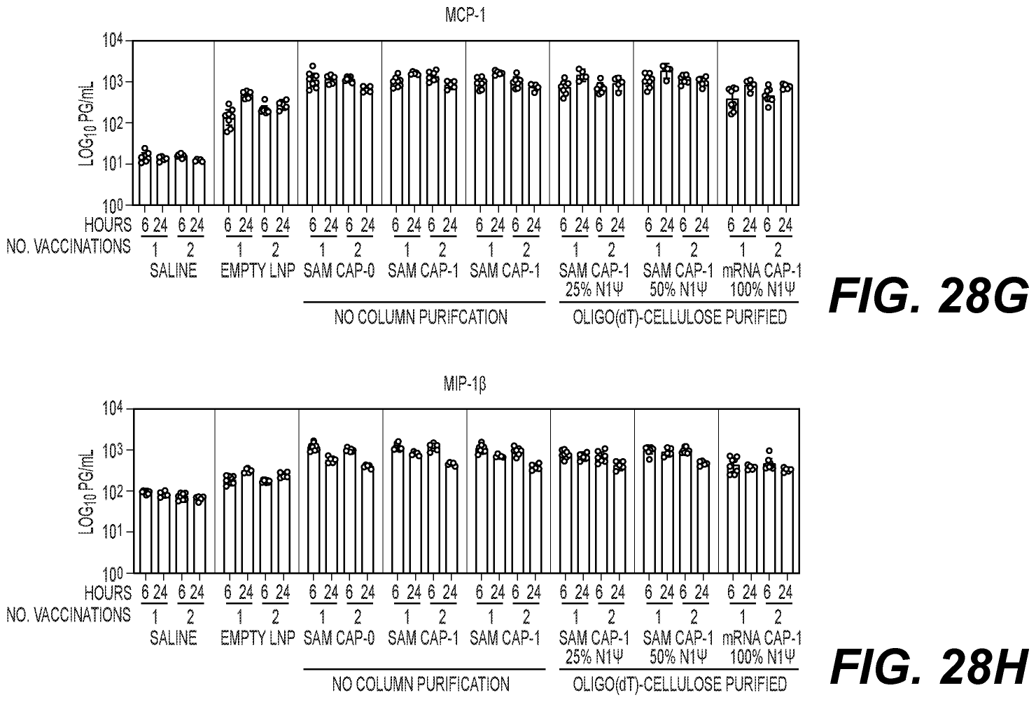

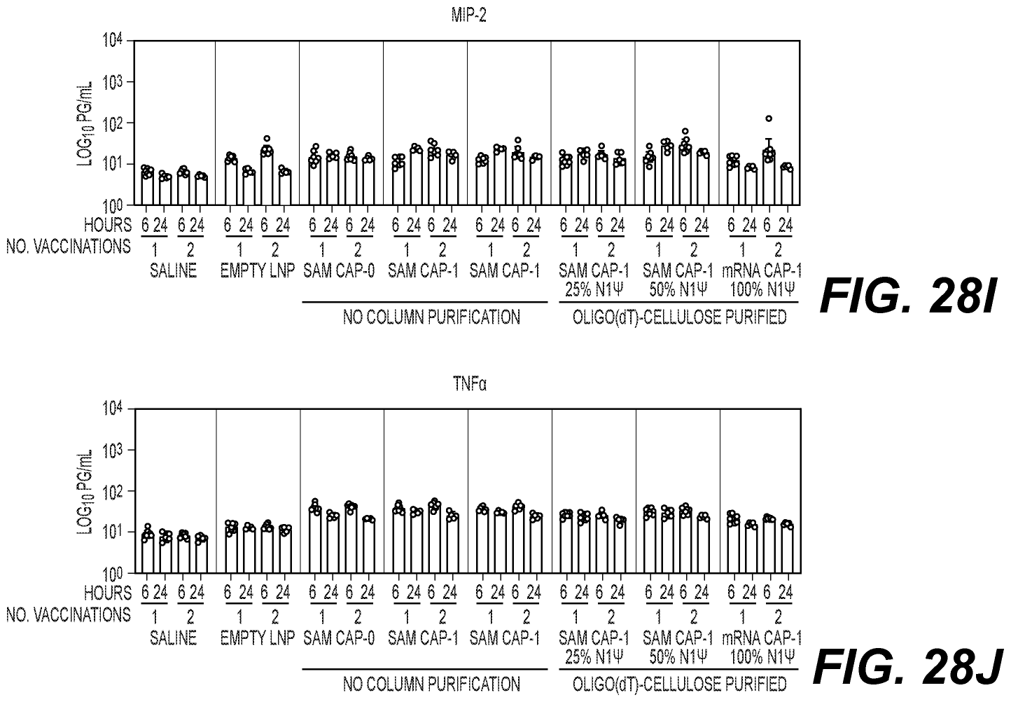

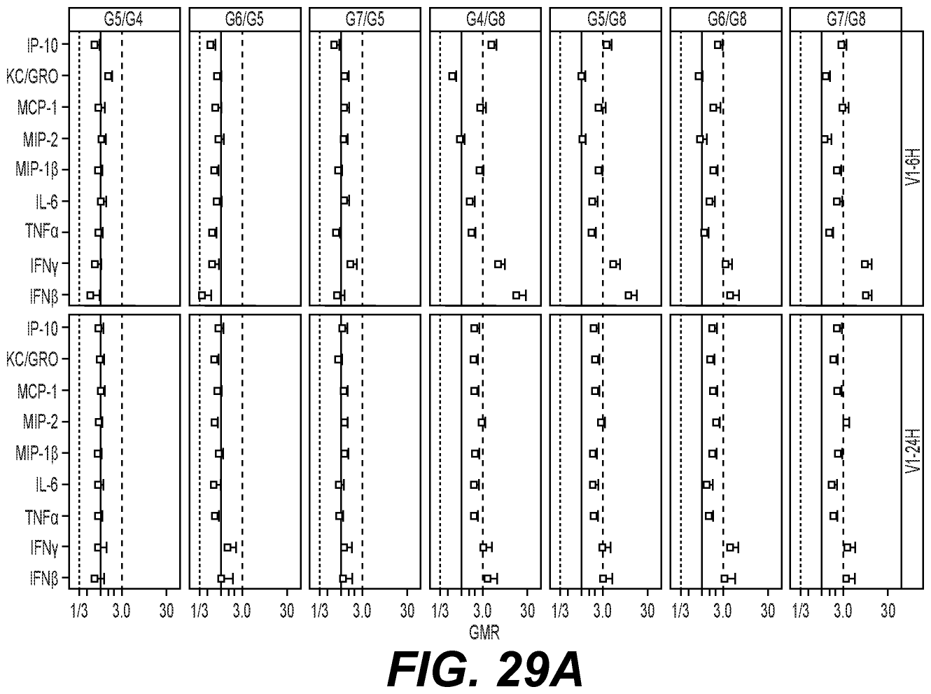

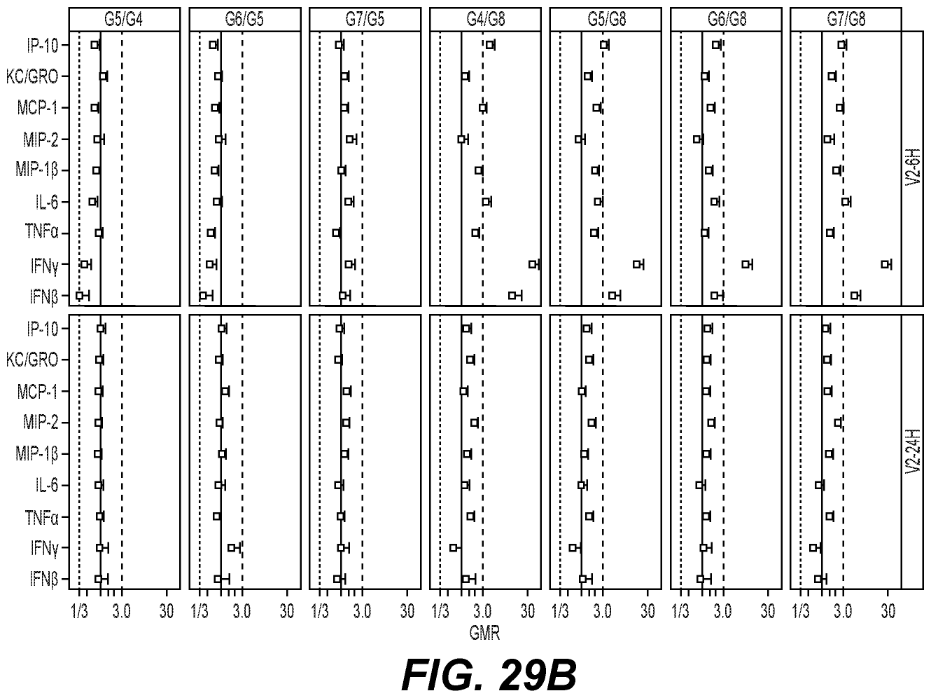

FIGS. 26, 27A, 27B, 27C, and 27D show SARS-CoV-2 neutralizing antibody titers (NT50) from animals treated on days 0 and 21 with the indicated LNP encapsulated RNAs (n=13 for saline, empty LNP, and the 3 μg unit dose groups and n=10 for the 0.15 μg unit dose groups). Data are transformed into log 10 NT50 and error bars are displayed with a 95% confidence interval. The lower limit of quantitation=25 (horizontal dashed line). Cap-1 5′ capping increased neutralizing antibody titers compared to that with cap-0 5′ capping. Oligo(dT)-cellulose purification did not increase neutralizing antibody titers. With the exception of the cap-0 capped unsubstituted SAM RNA, administration at a higher unit dose of, for example, 3.0 μg of SAM RNA, with or without N1-methylpseudouridine substitution, significantly increased neutralizing antibody titers when compared to those from administration of mRNA (non-replicating) which comprises N1-methylpseudouridine but not uridines. At a lower unit dose of, for example, 0.15 μg, administration of SAM RNA, with or without N1-methylpseudouridine substitution, significantly increased neutralizing antibody titers when compared to those from administration of mRNA (non-replicating) which comprises N1-methylpseudouridine but not uridines.

### Serum Cytokines

As noted above, serum was collected from mice in Groups 1-8 (i.e., the 3 μg unit doses) six hours (8 mice/group) and twenty-four hours (5 mice/group) after the first (i.e., day 0) and second (i.e., day 21) immunizations. Treatment with SAM comprising 25 mole % N1-methylpseudouridine and cap-1 capping reduced the levels of interferon-β (IFN-β) six hours after the first and second vaccination when compared to IFN-β levels at the respective time points and respective vaccinations of SAM RNA comprising uridines but no N1-methylpseudouridines. Treatment with SAM comprising 25 mole % N1-methylpseudouridine and cap-1 capping trended towards (i.e., α<0.1) reduced the levels of interferon-γ (IFN-γ) six hours after the first and second vaccination when compared to IFN-γ levels at the respective time points and respective vaccinations of SAM RNA comprising uridines but no N1-methylpseudouridines.

### T Cells

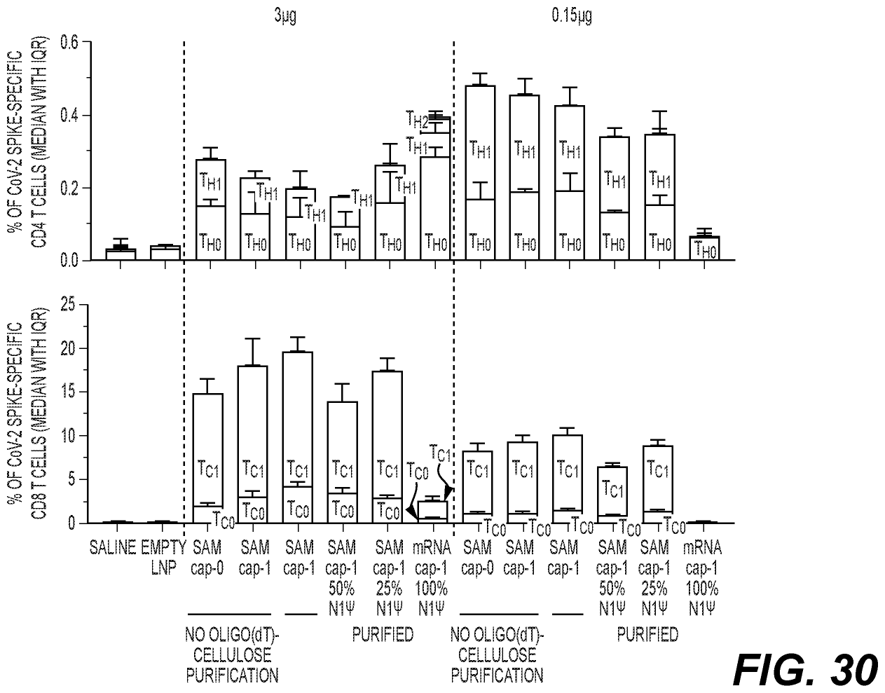

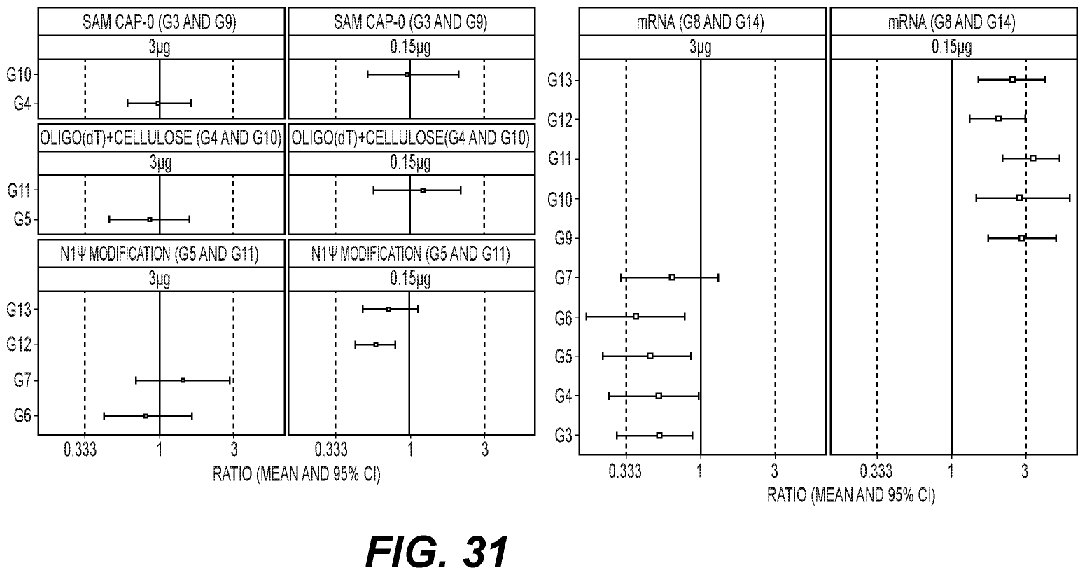

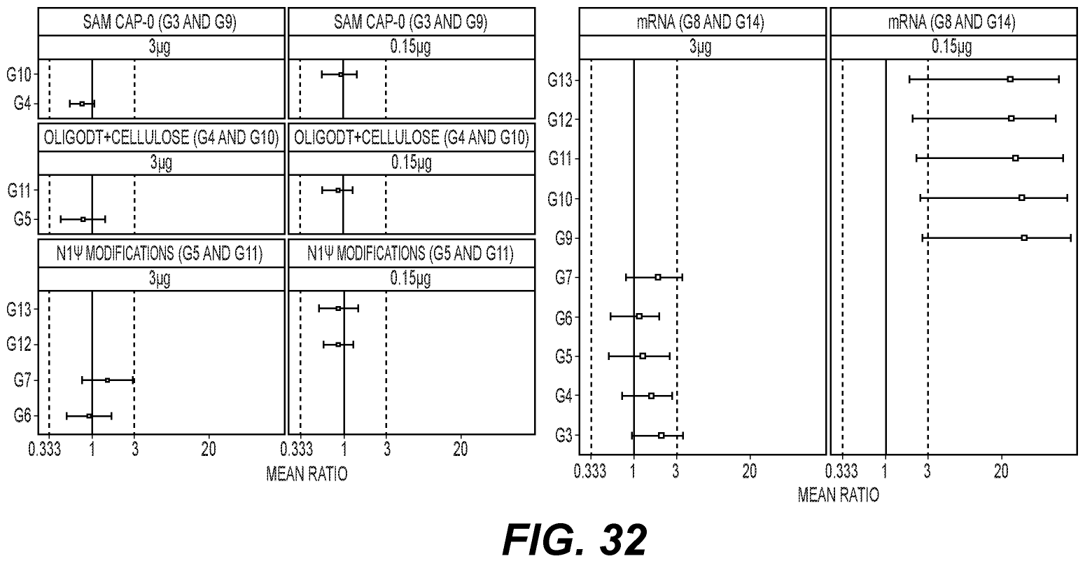

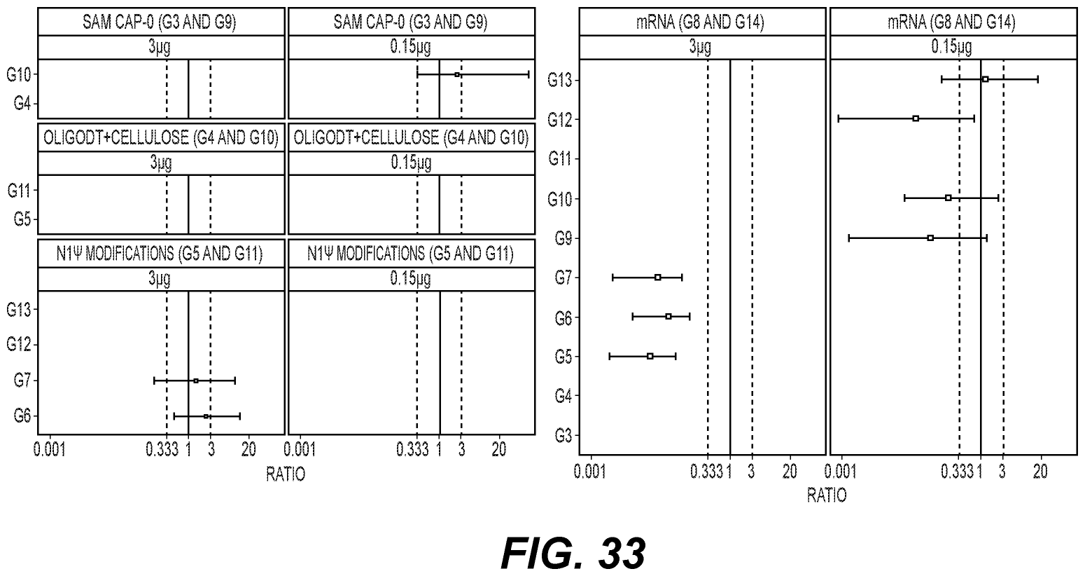

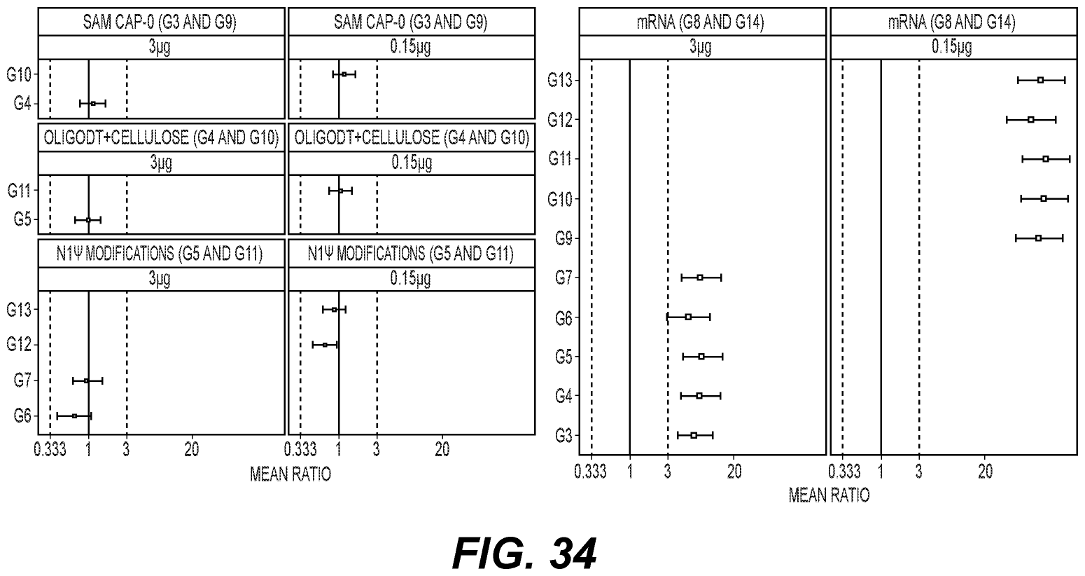

At a unit dose of for example 0.15 μg of RNA, the proportion of Th0, Th1, Tc0 and Tc1 was higher with the treatment with SAM RNA compared to those with the treatment of the non-replicating mRNA (group 14). At a unit dose of for example 3 μg of RNA, the proportion of Th0 and Th2 cells was lower with treatment of SAM RNA when compared to the proportion with treatment with non-replicating RNA. The proportion of Th1 appeared comparable between the SAM constructs and the mRNA vaccine. The proportion of Tc0 and Tc1 was higher with all SAM contracts compared to the mRNA group (G8). See FIGS. 30-34.

At a unit dose of for example 3 μg of RNA, treatment with RNA comprising N1 W did not impact the proportion of Th0, Th1, Th2, Tc0, and Tc1 T cells compared to proportion after the treatment with SAM RNA comprising uridines but no N1 W (group 5). At a unit dose of for example 0.15 μg of RNA, a marginal reduction in the proportion of Th0, Tc0 and Tc1 cells was observed in the 50% N1 W group (G12) when compared to the proportion after treatment with SAM RNA comprising uridines but no N1 W (group 5), while no other subpopulation was affected. See FIGS. 30-34.

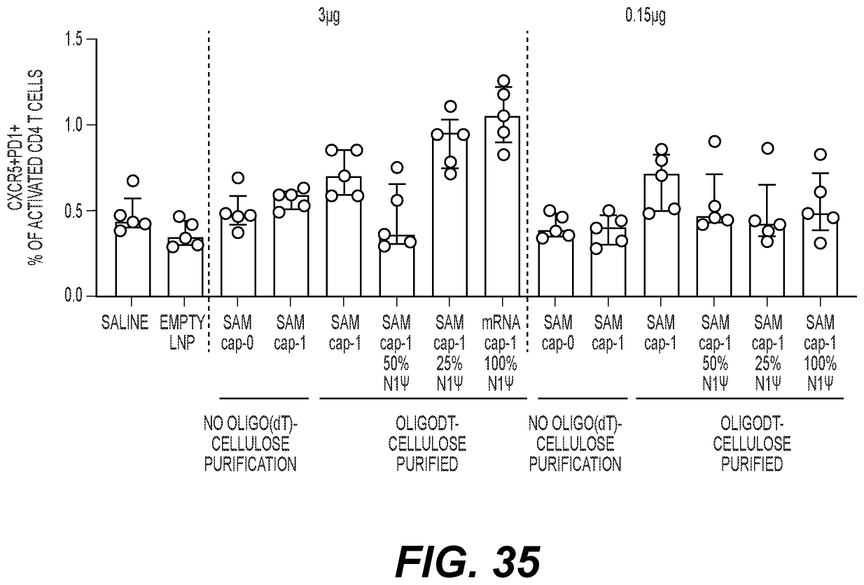

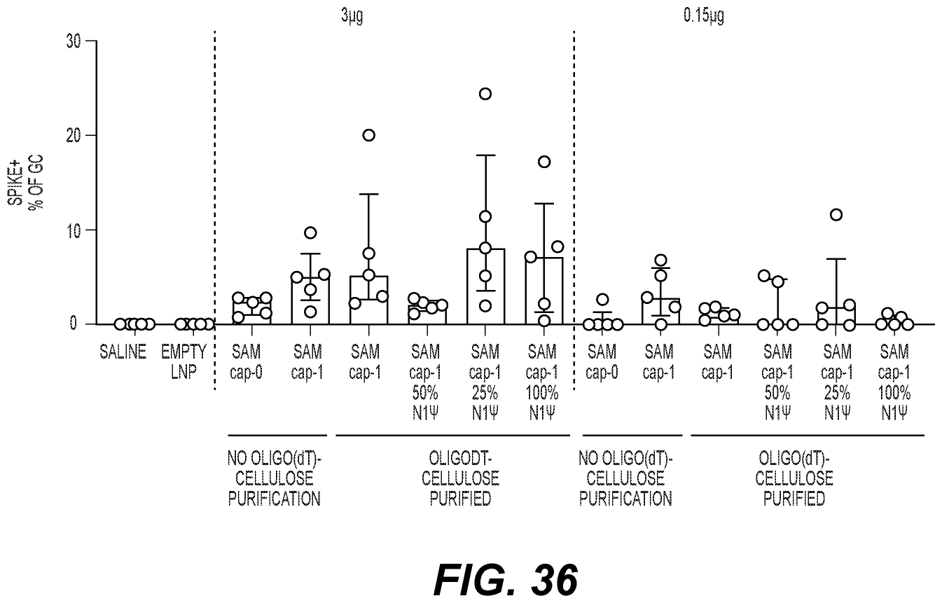

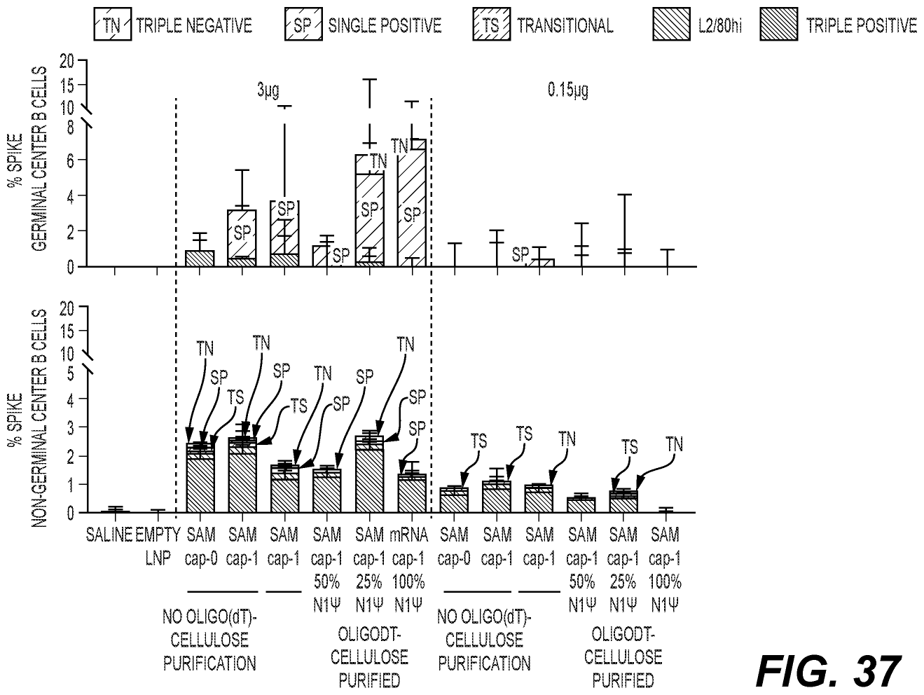

The proportion of follicular helper T cells (Tfh) cells (not antigen-specific) were measured in splenocytes harvested at day 35. These results are shown in FIG. 35. Treatment with unmodified SAM cap-1 oligo(dT)-cellulose purified (group 5 and group 11), SAM RNA comprising 25 mole % N1-methylpseudouridine (group 7), or non-replicating mRNA comprising N1-methylpseudourdine but not uridines (group 8) elicited significantly higher total Tfh responses compared to the saline control.

### SARS-CoV-2 Spike Protein-Specific B Cells

Treatment with 3 μg unit doses of SAM RNA comprising 25 mole % N1-methylpseudouridine elicited levels of B cells specific for SARS-CoV-2 spike protein that were comparable to those from treatment with non-replicating mRNA comprising N1-methylpseudouridine but not uridines.

### CONCLUSIONS

Example 10 demonstrates that not only can SAM RNA increase neutralizing and total antibody levels, and at lower doses, compared to fully N1-methylpseudouridine-substituted non-replicating RNA, but also treatment with 25 mole % N1-methylpseudouridine-substituted SAM RNA can at least reduce IFN-β levels in vivo when compared to the IFN-β levels obtained after treatment with unsubstituted SAM RNA. Cap-1, oligo(dT)-cellulose purified 50% N1-methylpseudouridine substitution elicited the lowest innate cytokines in the serum 6 h post-vaccination. These results were most similar to those obtained with treatment of non-replicating mRNA comprising N1-methylpseudouridine but no uridines. Treatment with RNA (SAM RNA and non-replicating RNA) comprising cap-1 capping significantly improved immunogenicity overall. Of the SAM RNA treatment groups, treatment with SAM RNA comprising 50 mole % N1-methylpseudouridines had the lowest innate cytokine activation profile. The treatment with SAM RNA comprising 25 mole % N1-methylpseudouridine sometimes resulted higher cytokine levels compared to those from treatment with SAM RNA comprising 50 mole % N1-methylpseudouridines. The treatment with SAM RNA comprising 25 mole % N1-methylpseudouridine resulted in higher measures of adaptive immune responses compared to those from treatment with SAM RNA comprising 50 mole % N1-methylpseudouridines. These measures of adaptive immune response that were elevated when compared to those from treatment with SAM RNA comprising 50 mole % N1-methylpseudouridines include in spike-protein specific antibody levels, neutralization antibody levels, the proportion of spike protein-specific T cells, total follicular helper T cells, and spike protein-specific B cell response. In addition, treatment with SAM RNA comprising 25 mole % N1-methylpseudouridine performed the best in eliciting single-positive CD73+ spike-specific B cells in the germinal center compared to treatment with SAM RNA comprising 50 mole % N1-methylpseudouridine, suggesting that the 25 mole % N1-methylpseudouridine was able to generate spike-specific cells that would continue to mature into long-lived memory B cells. The improvements observed with the treatment with SAM RNA comprising 25 mole % N1-methylpseudouridine over the treatment with SAM RNA comprising 50 mole % N1-methylpseudouridine do not foreclose the selection of 50 mole % N1-methylpseudouridine substitution within a critical range of 15 mole % to 60 mole % N1-methylpseudouridines of the total of uridines and N1-methylpseudouridines in the SAM RNA when it is determined that the lower levels of above-noted cytokines are preferred over the above-noted measures of adaptive immune responses.

### REFERENCES

All references cited in this document are incorporated by reference in their entirety for all purposes and for convenience have been listed below. The omission of any reference in the list below after being cited in this document does not negate the above-noted incorporation by reference. List of references:
NCBI (www.ncbi.nlm.nih.gov) Accession NOs: NM_001101.5, NG_056261.1, NM_000477.7, AH002618.2, NM_001686.4, U09278.2, NM_001291807.3, NR_168448.1, NG_007073.2, NR_152150.2, NG_029473.1, NM_153201.4, AH002842.2, NM_000586.4, NG_013080.2, NM_001961.4, NM 000558, OL521838, and NM_001130;
The World Health Organization: International Nonproprietary Names Programme. Messenger RNA Encoding the Full-Length SARS-CoV-2 Spike Glycoprotein; p. 11868;
Edo Kon, Uri Elia, and Dann Peer, “Principles for designing an optimal mRNA lipid nanoparticle vaccine,” Curr. Opin. Biotechnol. 2022, 73:329-336;
Harlow et al., 1999, In: Using Antibodies: A Laboratory Manual, Cold Spring Harbor Laboratory Press, NY;
Harlow et al., 1989, In: Antibodies: A Laboratory Manual, Cold Spring Harbor, N.Y.;
Houston et al., 1988, Proc. Natl. Acad. Sci. USA 85:5879-5883; Bird et al., 1988, Science 242:423-426;
Zhang et al., 2001, “Ionization behavior of amino lipids for siRNA delivery: determination of ionization constants, SAR, and the impact of lipid pKa on cationic lipid-biomembrane interactions, LANGMUIR 27: 1907-1914;
WO2005/113782;
WO2012/006376;
WO2012/030901;
WO2012/031046;
WO2012/031043;
WO2012/006378;
WO2011/076807;
WO2013/033563;
WO2013/006825;
WO2014/136086;
WO2015/095340;
WO2015/095346;
WO2016/037053;
WO2017/075531;
WO2018/081480:
WO2015/074085;
WO2018/170332;
WO2010/053572;
WO2012/170930;
U.S. Patent Application Publication NOs.: 20220081392, 20220072155, 20220040285, 20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, 20150376115, 20150140070A1, and 20100324120;
U.S. Application No. 61/905,724 and Ser. No. 15/614,499;
U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977, 10,106,490, 10,166,298, 10,221,127, 10,723,692, 11,040,112, 11,168,051, and 11,285,222; and
Geall et al. (2012) PNAS USA. September 4; 109(36): 14604-9.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, publication of a patent application, and publication cited herein are hereby incorporated herein by reference in its entirety. While this invention has been disclosed with reference to specific embodiments, it is apparent that other embodiments and variations of this invention may be devised without departing from the true spirit and scope of the invention. The appended claims are intended to be construed to include all such embodiments and equivalent variations.

### INTERPRETATION OF SEQUENCE LISTING

In the paper copy and ASCII-format electronic copy of the Sequence Listing, it is to be understood that any “U” or “u” (uridine) depicted therein may be replaced with an N1Ψ(N1-methylpseudouridine), and that the percentage “u” replaced with “N1Ψ” (substitution) corresponds with those described in the embodiments above. For example, a mole percentage of the N1-methylpseudouridines to the total of the N1-methylpseudouridines and the uridines of 50% contemplates and supports substitution of 50% of the “u” with “N1Ψ.” For example, a mole percentage of the N1-methylpseudouridines to the total of the N1-methylpseudouridines and the uridines of 25% contemplates and supports substitution of 25% of the “u” with “N1Ψ.” For example, a mole percentage of the N1-methylpseudouridines to the total of the N1-methylpseudouridines and the uridines of 75% contemplates and supports substitution of 75% of the “u” with “N1Ψ.”

1. A self-amplifying messenger (SAM) ribonucleic acid (RNA) comprising N1-methylpseudouridines, uridines, a first RNA segment, and a second RNA segment; the first RNA segment comprising a heterologous nucleic acid; the second RNA segment encoding one or more proteins capable of replicating the SAM RNA in an intracellular environment; the SAM RNA having a first mole percentage of the N1-methylpseudouridines to the total of the N1-methylpseudouridines and the uridines from 15% to 75%.

2. The SAM RNA of claim 1, the first mole percentage being to 70%.

3. The SAM RNA of claim 1, the first mole percentage being to 65%.

4. The SAM RNA of claim 1, the first mole percentage being to 60%.

5. The SAM RNA of claim 1, the first mole percentage being to 55%.

6. The SAM RNA of claim 1, the first mole percentage being to 50%.

7. The SAM RNA of claim 4, the first mole percentage being from 20%.

8. The SAM RNA of claim 4, the first mole percentage being from 25%.

9. The SAM RNA claim 1, the one or more proteins capable of replicating the SAM RNA in an intracellular environment comprising an alphavirus non-structural protein-1 (nsP1), an alphavirus nsP2, an alphavirus nsP3, and an alphavirus nsP4.

10. The SAM RNA claim 1, the heterologous nucleic acid encoding a heterologous protein.

11. The SAM RNA of claim 10, the heterologous protein comprising an immunogen, an antibody, or an immunotherapeutic molecule.

12. The SAM RNA of claim 10, the heterologous protein comprising an immunogen or an antibody against the immunogen.

13. The SAM RNA of claim 1 further comprising a poly-adenosine monophosphate (poly(A)) tail.

14. The SAM RNA of claim 13 further comprising a 5′ untranslated region (5′ UTR), the 5′ UTR being 5′ of the first RNA segment and the second RNA segment.

15. The SAM RNA of claim 14 further comprising a 3′ untranslated region (3′ UTR), the 3′ UTR being 3′ of the first RNA segment and the second RNA segment, and being 5′ of the poly(A) tail.

16. The SAM RNA of claim 15 further comprising a 5′ cap.

17. The SAM RNA of claim 16, the 5′ cap being a cap-0, a cap-1, or a cap-2.

18. The SAM RNA of claim 16, the 5′ cap being a cap-1.

19. The SAM RNA of claim 16, the 5′ cap being a cap-0.

20. A composition comprising the SAM RNA of claim 1 and a pharmaceutically acceptable delivery vehicle.

21. The composition of claim 20, the pharmaceutically acceptable delivery vehicle comprising a lipid nanoparticle (LNP).

22. The composition of claim 21, the LNP encapsulating the SAM RNA.

23. A method of eliciting an immune response in a subject to the immunogen, the method comprising administering to the subject an effective amount of the SAM RNA of claim 12.

24. The method of claim 23, the immune response being a protective immune response.

25. The method of claim 23, the immune response being a therapeutic immune response.

26. The method of claim 23, the immunogen comprising a venom, a poison, an allergen, a cancer antigen, a bacterial antigen, a viral antigen, a fungal antigen, a parasite antigen, or a fragment thereof.

27. The method of claim 23, the heterologous protein comprising the antibody against the immunogen.

28. The method of claim 23, the heterologous protein comprising the immunogen.

29. The method of claim 23, the subject being human.

30. A method of manufacturing the SAM RNA of claim 1, the method comprising admixing an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the admixing being under conditions wherein the RNA polymerase produces the SAM RNA from the template nucleic acid.

31. A method of manufacturing the SAM RNA of claim 16, the method comprising:
a first admixing of an RNA polymerase, the N1-methylpseudouridines, the uridines, and a template nucleic acid comprising a sequence of the SAM RNA, thereby obtaining an admixture; the admixture having a second mole percentage of the N1-methylpseudouridines to the total of the N1-methylpseudouridines and the uridines; the second mole percentage being the same as the first mole percentage; the first admixing being under conditions wherein the RNA polymerase produces the SAM RNA from the template nucleic acid, thereby obtaining an uncapped SAM RNA; and
a second admixing of the uncapped SAM RNA, a messenger RNA guanylyltransferase, guanosine triphosphate, a (guaninine-N7-)-methyltransferase, and S-adenosyl-L-methionine, the second admixing being under conditions that cause a 5′ to 5′ triphosphate cap linkage, the second admixing optionally further comprising a 2′-O-methyltransferase, and optionally the second admixing being under conditions that form cap-1 or cap-2.

32. The method of claim 30, the RNA polymerase being a T7 RNA polymerase.

33. A method of eliciting an immune response in a subject to an immunogen, the method comprising administering to the subject an effective amount of the composition of claim 20, and the heterologous nucleic acid encoding the immunogen or an antibody against the immunogen.

34. A method of eliciting an immune response in a subject to an immunogen, the method comprising administering to the subject an effective amount of the composition of claim 22, and the heterologous nucleic acid encoding the immunogen or an antibody against the immunogen.

35. A plurality of auto-amplifying messenger (AAM) RNAs comprising N1-methylpseudouridines, uridines, a first RNA, and one or more second RNA; the first RNA comprising a heterologous nucleic acid; the one or more second RNA encoding one or more proteins capable of replicating the AAM RNA in an intracellular environment; the plurality of AAM RNAs having a first mole percentage of the N1-methylpseudouridines to the total of the N1-methypseudouridines and uridines from 15% to 75%.

36. A composition comprising the AAM RNA of claim 35 and a pharmaceutically acceptable delivery vehicle.

37. The composition of claim 36, the pharmaceutically acceptable delivery vehicle comprising a LNP.

38. A method of eliciting an immune response in a subject to an immunogen, the method comprising administering to the subject an effective amount of the plurality of AAM RNAs of claim 35; the heterologous nucleic acid encoding an immunogen or an antibody against the immunogen.

39. A method of manufacturing the plurality of AAM RNAs of claim 35, the method comprising one or more admixing of an RNA polymerase, the N1-methylpseudouridines, the uridines, and one or more template nucleic acids, thereby obtaining an admixture wherein the one or more template nucleic acids comprise a sequence of the plurality of AAM RNAs; the one or more admixture having a second mole percentage of the N1-methylpseudouridines to the total of the N1-methylpseudouridines and the uridines; the second mole percentage being the same as the first mole percentage; the one or more admixing being under conditions wherein the RNA polymerase produces the plurality of AAM RNAs from the one or more template nucleic acids.
